(19) United States
(12) Patent Application Publication
(10) Pub. No.: US 2023/0357735 A1
(43) Pub. Date: Nov. 9, 2023

(54) PROGRAMMABLE NUCLEASES AND METHODS OF USE

(71) Applicant: Mammoth Biosciences, Inc., Brisbane, CA (US)

(72) Inventors: Lucas Benjamin HARRINGTON, San Francisco, CA (US); William Douglass WRIGHT, Fairfield, CA (US); Pei-Qi LIU, Oakland, CA (US); Benjamin Julius RAUCH, Oakland, CA (US); Wiputra Jaya HARTONO, San Francisco, CA (US); Bridget Ann Paine MCKAY, Portola Valley, CA (US); Danuta Sastre PHIPPS, San Mateo, CA (US); Yuxuan ZHENG, San Jose, CA (US); Nerea SANVISENS, San Francisco, CA (US); Sean CHEN, Berkeley, CA (US); David PAEZ-ESPINO, Concord, CA (US)

(21) Appl. No.: 18/000,640

(22) PCT Filed: Jun. 3, 2021
(86) PCT No.: PCT/US21/35781

§ 371 Date: Dec. 2, 2022

Related U.S. Application Data
(60) Provisional application No. 63/034,346, filed on Jun. 3, 2020.
(60) Provisional application No. 63/037,535, filed on Jun. 10, 2020.
(60) Provisional application No. 63/040,998, filed on Jun. 18, 2020.
(60) Provisional application No. 63/092,481, filed on Oct. 15, 2020.
(60) Provisional application No. 63/116,083, filed on Nov. 19, 2020.
(60) Provisional application No. 63/124,676, filed on Dec. 11, 2020.
(60) Provisional application No. 63/156,883, filed on Mar. 4, 2021.
(60) Provisional application No. 63/178,472, filed on Apr. 22, 2021.

(51) Int. Cl. C12N 9/22; C12N 15/11; C12N 15/90; C12Q 1/6897

(52) U.S. Cl. CPC C12N 9/22; C12N 15/11; C12N 15/907; C12Q 1/6897; C12N 2310/20; C12N 2800/80

(57) ABSTRACT

Provided herein, in certain embodiments, are programmable nucleases, guide nucleic acids, and complexes thereof. Certain programmable nucleases provided herein comprise a RuvC domain. Also provided herein are nucleic acids encoding said programmable nucleases and guide nucleic acids. Also provided herein are methods of genome editing, methods of regulating gene expression, and methods of detecting nucleic acids with said programmable nucleases and guide nucleic acids.

### CROSS-REFERENCE

The present application claims priority to and benefit from U.S. Provisional Application No. 63/034,346, filed on Jun. 3, 2020, U.S. Provisional Application No. 63/037,535, filed on Jun. 10, 2020, U.S. Provisional Application No. 63/040,998, filed on Jun. 18, 2020, U.S. Provisional Application No. 63/092,481, filed on Oct. 15, 2020, U.S. Provisional Application No. 63/116,083, filed on Nov. 19, 2020, U.S. Provisional Application No. 63/124,676, filed on Dec. 11, 2020, U.S. Provisional Application No. 63/156,883, filed on Mar. 4, 2021, and U.S. Provisional Application No. 63/178,472, filed on Apr. 22, 2021, the entire contents of each of which are herein incorporated by reference.

### BACKGROUND

Certain programmable nucleases can be used for genome editing of nucleic acid sequences or detection of nucleic acid sequences. There is a need for high efficiency, programmable nucleases that are capable of working under various sample conditions and can be used for both genome editing and diagnostics.

### SUMMARY

In various aspects, the present disclosure provides a composition comprising: a) a programmable CasΦ nuclease or a nucleic acid encoding said programmable CasΦ nuclease, wherein said programmable CasΦ nuclease comprises at least 85% sequence identity to a sequence selected from the group consisting of SEQ ID NOs: 1 to 47, SEQ ID NO. 105, and SEQ ID NO. 107, and b) a guide nucleic acid or a nucleic acid encoding said guide nucleic acid, wherein said guide nucleic acid comprises a region comprising a nucleotide sequence that is complementary to a target nucleic acid sequence and an additional region, wherein said region and said additional region are heterologous to each other.

In some aspects, the additional region of the guide nucleic acid comprises at least 85% sequence identity to a sequence selected from the group consisting of SEQ ID NOs: 48 to 86. In some aspects, the guide nucleic acid comprises a sequence comprising at least 95% sequence identity to a sequence selected from the group consisting of SEQ ID NOs: 48 to 86. In some aspects, the guide nucleic acid comprises a sequence selected from the group consisting of SEQ ID NOs: 48 to 86. In some aspects, the programmable CasΦ nuclease comprises nickase activity. In some aspects, the programmable CasΦ nuclease comprises double-strand cleavage activity. In some aspects, the programmable CasΦ nuclease comprises at least 90% sequence identity to a sequence selected from the group consisting of SEQ ID NOs: 1 to 47, SEQ ID NO. 105, and SEQ ID NO. 107.

In some aspects, the programmable CasΦ nuclease comprises at least 95% sequence identity to a sequence selected from the group consisting of SEQ ID NOs: 1 to 47, SEQ ID NO. 105, and SEQ ID NO. 107. In some aspects, the programmable CasΦ nuclease comprises at least 98% sequence identity to a sequence selected from the group consisting of SEQ ID NOs: 1 to 47, SEQ ID NO. 105, and SEQ ID NO. 107. In some aspects, the programmable CasΦ nuclease comprises a sequence selected from the group consisting of SEQ ID NOs: 1 to 47, SEQ ID NO. 105, and SEQ ID NO. 107. In some aspects, the guide nucleic acid does not comprise a tracrRNA. In some aspects, the programmable CasΦ nuclease does not require a tracrRNA. In some aspects, the programmable CasΦ nuclease comprises greater nickase activity when complexed with the guide nucleic acid at a temperature from about 20° C. to about 25° C., as compared with complex formation at a temperature of about 37° C. In some aspects, the guide nucleic acid comprises at least 98% sequence identity to SEQ ID NO: 54. In some aspects, the guide nucleic acid comprises at least 98% sequence identity to SEQ ID NO: 57. In some aspects, the programmable CasΦ nuclease comprises greater nickase activity when complexed with the guide nucleic acid comprising a sequence comprising at least 98% sequence identity to SEQ ID NO: 57, as compared to when complexed with a guide nucleic acid comprising SEQ ID NO: 49.

In some aspects, the programmable CasΦ nuclease exhibits greater nicking activity as compared to double stranded cleavage activity. In some aspects, the programmable CasΦ nuclease exhibits greater double stranded cleavage activity as compared to nicking activity. In some aspects, the programmable CasΦ nuclease comprises a single active site in a RuvC domain that is capable of catalyzing pre-crRNA processing and nicking or cleaving of nucleic acids. In some aspects, the programmable CasΦ nuclease recognizes a protospacer adjacent motif (PAM) of 5′-TBN-3′, wherein B is one or more of C, G, or, T. In some aspects, the programmable CasΦ nuclease recognizes a protospacer adjacent motif (PAM) of 5′-TTTN-3′.

In various aspects, the present disclosure provides a method of modifying a target nucleic acid sequence, the method comprising: contacting a target nucleic acid sequence with a programmable CasΦ nuclease comprising at least 85% sequence identity to a sequence selected from the group consisting of SEQ ID NOs: 1 to 47, SEQ ID NO. 105, and SEQ ID NO. 107, and a guide nucleic acid, wherein the programmable CasΦ nuclease cleaves the target nucleic acid sequence, thereby modifying the target nucleic acid sequence.

In some aspects, the programmable CasΦ nuclease introduces a double-stranded break in the target nucleic acid sequence. In some aspects, the programmable CasΦ nuclease comprises double-strand cleavage activity. In some aspects, the programmable CasΦ nuclease cleaves a single-strand of the target nucleic acid sequence. In some aspects, the programmable CasΦ nuclease comprises nickase activity. In some aspects, the programmable CasΦ nuclease exhibits greater nicking activity as compared to double stranded cleavage activity. In some aspects, the programmable CasΦ nuclease exhibits greater double stranded cleavage activity as compared to nicking activity. In some aspects, the target nucleic acid is DNA. In some aspects, the target nucleic acid is double-stranded DNA. In some aspects, the programmable CasΦ nuclease cleaves a non-target strand of the double-stranded DNA, wherein the non-target strand is non-complementary to the guide nucleic acid. In some aspects, the programmable CasΦ nuclease does not cleave a target strand of the double-stranded DNA, wherein the target strand is complementary to the guide nucleic acid.

In some aspects, the programmable CasΦ nuclease comprises at least 90% sequence identity to a sequence selected from the group consisting of SEQ ID NOs: 1 to 47, SEQ ID NO. 105, and SEQ ID NO. 107. In some aspects, the programmable CasΦ nuclease comprises at least 95% sequence identity to a sequence selected from the group consisting of SEQ ID NOs: 1 to 47, SEQ ID NO. 105, and SEQ ID NO. 107. In some aspects, the programmable CasΦ nuclease comprises at least 98% sequence identity to a sequence selected from the group consisting of SEQ ID NOs: 1 to 47, SEQ ID NO. 105, and SEQ ID NO. 107. In some aspects, the programmable CasΦ nuclease comprises a sequence selected from the group consisting of SEQ ID NOs: 1 to 47, SEQ ID NO. 105, and SEQ ID NO. 107. In some aspects, the guide nucleic acid comprises a sequence comprising at least 85% sequence identity to a sequence selected from the group consisting of SEQ ID NOs: 48 to 86. In some aspects, the guide nucleic acid comprises a sequence comprising at least 95% sequence identity to a sequence selected from the group consisting of SEQ ID NOs: 48 to 86. In some aspects, the guide nucleic acid comprises a sequence selected from the group consisting of SEQ ID NOs: 48 to 86.

In some aspects, the guide nucleic acid does not comprise a tracrRNA. In some aspects, the target nucleic acid sequence comprises a mutated sequence or a sequence associated with a disease. In some aspects, the mutated sequence is removed after the programmable CasΦ nuclease cleaves the target nucleic acid sequence. In some aspects, the target nucleic acid sequence is in a human cell. In some aspects, the method is performed in vivo. In some aspects, the method is performed ex vivo. In some aspects, the method further comprises inserting a donor polynucleotide into the target nucleic acid sequence at the site of cleavage.

In various aspects, the present disclosure provides a method of introducing a break in a target nucleic acid, the method comprising: contacting the target nucleic acid with: (a) a first guide nucleic acid comprising a region that binds to a first programmable nickase comprising at least 85% sequence identity to a sequence selected from the group consisting of SEQ ID NOs: 1 to 47, SEQ ID NO. 105, and SEQ ID NO. 107; and (b) a second guide nucleic acid comprising a region that binds to a second programmable nickase comprising at least 85% sequence identity to a sequence selected from the group consisting of SEQ ID NOs: 1 to 47, SEQ ID NO. 105, and SEQ ID NO. 107, wherein the first guide nucleic acid comprises a first additional region that binds to the target nucleic acid and wherein the second guide nucleic acid comprises a second additional region that binds to the target nucleic acid and wherein the first additional region of the first guide nucleic acid and the second additional region of the second guide nucleic acid bind opposing strands of the target nucleic acid. In some aspects, the first programmable nickase, the second programmable nickase, or both comprise at least 90% sequence identity to a sequence selected from the group consisting of SEQ ID NOs: 1 to 47, SEQ ID NO. 105, and SEQ ID NO. 107.

In some aspects, the first programmable nickase, the second programmable nickase, or both comprise at least 95% sequence identity to a sequence selected from the group consisting of SEQ ID NOs: 1 to 47, SEQ ID NO. 105, and SEQ ID NO. 107. In some aspects, the first programmable nickase, the second programmable nickase, or both comprise a sequence selected from the group consisting of SEQ ID NOs: 1 to 47, SEQ ID NO. 105, and SEQ ID NO. 107. In some aspects, the first guide nucleic acid, the second guide nucleic acid, or both comprise a sequence comprising at least 85% sequence identity to a sequence selected from the group consisting of SEQ ID NOs: 48 to 86. In some aspects, the first guide nucleic acid, the second guide nucleic acid, or both comprise a sequence comprising at least 95% sequence identity to a sequence selected from the group consisting of SEQ ID NOs: 48 to 86. In some aspects, the first guide nucleic acid, the second guide nucleic acid, or both comprise a sequence selected from the group consisting of SEQ ID NOs: 48 to 86.

In some aspects, the first programmable nickase and the second programmable nickase exhibit greater nicking activity as compared to double stranded cleavage activity. In some aspects, the first programmable nickase and the second programmable nickase nick the target nucleic acid at two different sites. In some aspects, the target nucleic acid comprises double stranded DNA. In some aspects, the two different sites are on opposing strands of the double stranded DNA. In some aspects, the target nucleic acid comprises a mutated sequence or a sequence is associated with a disease. In some aspects, the mutated sequence is removed after the first programmable nickase and the second programmable nickase nick the target nucleic acid. In some aspects, the target nucleic acid is in a cell. In some aspects, the method is performed in vivo. In some aspects, the method is performed ex vivo. In some aspects, the first programmable nickase and the second programmable nickase are the same. In some aspects, the first programmable nickase and the second programmable nickase are different.

In various aspects, the present disclosure provides a method of detecting a target nucleic acid in a sample, the method comprising contacting a sample comprising a target nucleic acid with (a) a programmable CasΦ nuclease comprising at least 85% sequence identity to a sequence selected from the group consisting of SEQ ID NOs: 1 to 47, SEQ ID NO. 105, and SEQ ID NO. 107; (b) a guide RNA comprising a region that binds to the programmable CasΦ nuclease and an additional region that binds to the target nucleic acid; and (c) a labeled single stranded DNA reporter that does not bind the guide RNA; cleaving the labeled single stranded DNA reporter by the programmable CasΦ nuclease to release a detectable label; and detecting the target nucleic acid by measuring a signal from the detectable label.

In some aspects, the target nucleic acid is single stranded DNA. In some aspects, the target nucleic acid is double stranded DNA. In some aspects, the target nucleic acid is a viral nucleic acid. In some aspects, the target nucleic acid is bacterial nucleic acid. In some aspects, the target nucleic acid is from a human cell. In some aspects, the target nucleic acid is a fetal nucleic acid. In some aspects, the sample is derived from a subject's saliva, blood, serum, plasma, urine, aspirate, or biopsy sample. In some aspects, the programmable CasΦ nuclease comprises at least 95% sequence identity to a sequence selected from the group consisting of SEQ ID NOs: 1 to 47, SEQ ID NO. 105, and SEQ ID NO. 107. In some aspects, the programmable CasΦ nuclease comprises a sequence selected from the group consisting of SEQ ID NOs: 1 to 47, SEQ ID NO. 105, and SEQ ID NO. 107.

In some aspects, the guide RNA comprises at least about 95% sequence identity to a sequence selected from the group consisting of SEQ ID NOs: 48 to 86. In some aspects, the guide RNA comprises a sequence selected from the group consisting of SEQ ID NOs: 48 to 86. In some aspects, the sample comprises a phosphate buffer, a Tris buffer, or a HEPES buffer. In some aspects, the sample comprises a pH of 7 to 9. In some aspects, the sample comprises a pH of 7.5 to 8. In some aspects, the sample comprises a salt concentration of 25 nM to 200 mM. In some aspects, the single stranded DNA reporter comprises an ssDNA-fluorescence quenching DNA reporter. In some aspects, the ssDNA-fluorescence quenching DNA reporter is a universal ssDNA-fluorescence quenching DNA reporter. In some aspects, the programmable CasΦ nuclease exhibits PAM-independent cleaving.

In various aspects, the present disclosure provides a method of modulating transcription of a gene in a cell, the method comprising: introducing into a cell comprising a target nucleic acid sequence: (i) a fusion polypeptide or a nucleic acid encoding the fusion polypeptide, wherein the fusion polypeptide comprises: (a) a dCasΦ polypeptide comprising at least 85% sequence identity to a sequence selected from the group consisting of SEQ ID NOs: 1 to 47, SEQ ID NO. 105, and SEQ ID NO. 107, wherein the dCasΦ polypeptide is enzymatically inactive; and (b) a polypeptide comprising transcriptional regulation activity; and (ii) a guide nucleic acid, or a nucleic acid comprising a nucleotide sequence encoding the guide nucleic acid, wherein the guide nucleic acid comprises a region that binds to the dCasΦ polypeptide and an additional region that binds to the target nucleic acid; wherein transcription of the gene is modulated through the fusion polypeptide acting on the target nucleic acid sequence.

In some aspects, the dCasΦ polypeptide comprises at least 95% sequence identity to a sequence selected from the group consisting of SEQ ID NOs: 1 to 47, SEQ ID NO. 105, and SEQ ID NO. 107. In some aspects, the guide nucleic acid comprises at least about 95% sequence identity to a sequence selected from the group consisting of SEQ ID NOs: 48 to 86. In some aspects, the guide nucleic acid comprises a sequence selected from the group consisting of SEQ ID NOs: 48 to 86. In some aspects, the guide nucleic acid comprises a sequence selected from the group consisting of SEQ ID NOs: 48 to 86. In some aspects, the polypeptide comprising transcriptional regulation activity polypeptide comprises transcription activation activity.

In some aspects, the polypeptide comprising transcriptional regulation activity polypeptide comprises transcription repressor activity. In some aspects, the polypeptide comprising transcriptional regulation activity polypeptide comprises an activity selected from the group consisting of transcription activation activity, transcription repression activity, nuclease activity, transcription release factor activity, histone modification activity, histone acetyltransferase activity, nucleic acid association activity, DNA methylase activity, direct or indirect DNA demethylase activity, methyltransferase activity, demethylase activity, acetyltransferase activity, deacetylase activity, kinase activity, phosphatase activity, ubiquitin ligase activity, deubiquitinating activity, adenylation activity, deadenylation activity, deaminase activity, SUMOylating activity, deSUMOylating activity, ribosylation activity, deribosylation activity, myristoylation activity, and demyristoylation activity.

In various aspects, the present disclosure provides a composition comprising: a) a Cas nuclease or nucleic acid encoding said Cas nuclease, and b) a guide nucleic acid or a nucleic acid encoding said guide nucleic acid, wherein said guide nucleic acid comprises a region comprising a nucleotide sequence that is complementary to a target nucleic acid sequence and an additional region, wherein said region and said additional region are heterologous to each other; wherein the Cas nuclease comprises a RuvC domain, wherein the RuvC domain is capable of processing a pre-crRNA and cleaving a target nucleic acid. In some aspects, the same active site in the RuvC domain catalyzes the processing of the pre-crRNA and the cleaving of the target nucleic acid. In some aspects, the Cas nuclease is the programmable CasΦ nuclease as disclosed herein. In some aspects, the Cas nuclease recognizes a protospacer adjacent motif (PAM) of 5′-TBN-3′, wherein B is one or more of C, G, or, T. In some aspects, the Cas nuclease recognizes a protospacer adjacent motif (PAM) of 5′-TTTN-3′. In some aspects, the Cas nuclease recognizes a protospacer adjacent motif (PAM) of 5′-TTN-3′. In some aspects, the Cas nuclease recognizes a protospacer adjacent motif (PAM) of 5′-GTTB-3′, wherein B is C, G, or T. In some aspects, the Cas nuclease recognizes a protospacer adjacent motif (PAM) of 5′-GTTK-3′, 5′-VTTK-3′, 5′-VTTS-3′, 5′-TTTS-3′ or 5′-VTTN-3′, where K is G or T, V is A, C or G, and S is C or G. In some aspects, the composition is used in any of the above methods.

In various aspects, the present disclosure provides the use of a programmable CasΦ nuclease to modify a target nucleic acid sequence according to any one of the above methods. In various aspects, the present disclosure provides the use of a first programmable nickase and a second programmable nickase to introduce a break in a target nucleic acid according to any one of the above methods. In various aspects, the present disclosure provides the use of a programmable CasΦ nuclease to detect a target nucleic acid in a sample according to any one of the above methods. In various aspects, the present disclosure provides the use of a dCasΦ polypeptide to modulate transcription of a gene in a cell according to any one of the above methods. In some aspects, the region is a spacer region and the additional region is a repeat region. In some aspects, the region is a repeat region and the additional region is a spacer region. In some aspects, the repeat region comprises a GAC sequence, optionally wherein the GAC sequence is at the 3′ end of the repeat region. In some aspects, the repeat region comprises a hairpin, optionally wherein the hairpin is in the 3′ portion of the repeat region. In some aspects, the hairpin comprises a double-stranded stem portion and a single-stranded loop portion. In some aspects, a strand of the stem portion comprises a CYC sequence and the other strand of the stem portion comprises a GRG sequence, wherein Y and R are complementary. In some aspects, the G of the GAC sequence is in the stem portion of the hairpin. In some aspects, each strand of the stem portion comprises 3, 4 or 5 nucleotides. In some aspects, the loop portion comprises between 2 and 8 nucleotides, optionally wherein the loop portion comprises 4 nucleotides. In some aspects, the guide nucleic acid comprises at least 98% sequence identity to SEQ ID NO: 54.

In some aspects, the repeat region is between 15 and 50 nucleotides in length, preferably, wherein the repeat region is between 19 and 37 nucleotides in length. In some aspects, the spacer region is between 15 and 50 nucleotides in length, between 15 and 40 nucleotides in length, or between 15 and 35 nucleotides in length, preferably wherein the spacer region is between 16 and 30 nucleotides in length. In some aspects, the spacer region is between 16 and 20 nucleotides in length. In some aspects, the programmable CasΦ nuclease forms a complex with a divalent metal ion, preferably wherein the divalent metal ion is Mg2+.

In various aspects, the present disclosure provides a programmable CasΦ nuclease or a nucleic acid encoding said programmable CasΦ nuclease, wherein said programmable CasΦ nuclease comprises at least 85% sequence identity to a sequence selected from the group consisting of SEQ ID NOs: 1 to 47, SEQ ID NO. 105, and SEQ ID NO. 107, and wherein the programmable CasΦ nuclease is capable of binding to a guide RNA comprising a first region that is complementary to a target nucleic acid sequence in a eukaryotic genome and a second region that binds to the programmable CasΦ nuclease; a complex comprising the programmable CasΦ nuclease and the guide RNA binds to the target sequence; the programmable CasΦ nuclease comprises a RuvC domain, wherein the RuvC domain is capable of processing a pre-crRNA and cleaving the target nucleic acid; and the programmable CasΦ nuclease does not require a tracrRNA to cleave the target nucleic acid.

In various aspects, the present disclosure provides a programmable CasΦ nuclease or a nucleic acid encoding said programmable CasΦ nuclease, wherein said programmable CasΦ nuclease comprises a RuvC-like domain which matches PFAM family PF07282 and does not match PFAM family PF18516, and wherein the programmable CasΦ nuclease is capable of binding to a guide RNA comprising a first region that is complementary to a target nucleic acid sequence in a eukaryotic genome and a second region that binds to the programmable CasΦ nuclease; a complex comprising the programmable CasΦ nuclease and the guide RNA binds to the target sequence; the RuvC-like domain is capable of processing a pre-crRNA and cleaving the target nucleic acid; and the programmable CasΦ nuclease does not require a tracrRNA to cleave the target nucleic acid.

In various aspects, the present disclosure provides a programmable CasΦ nuclease or a nucleic acid encoding said programmable CasΦ nuclease, wherein said programmable CasΦ nuclease comprises at least 85% sequence identity to a sequence selected from the group consisting of SEQ ID NOs: 1 to 47, SEQ ID NO. 105, or SEQ ID NO. 107, and wherein a) the programmable CasΦ nuclease comprises a RuvC-like domain which matches PFAM family PF07282 and does not match PFAM family PF18516; b) the programmable CasΦ nuclease is capable of binding to a guide RNA comprising a first region that is complementary to a target nucleic acid sequence in a eukaryotic genome and a second region that binds to the programmable CasΦ nuclease; c) a complex comprising the programmable CasΦ nuclease and the guide RNA binds to the target sequence; d) the RuvC-like domain is capable of processing a pre-crRNA and cleaving the target nucleic acid; and e) the programmable CasΦ nuclease does not require a tracrRNA to cleave the target nucleic acid.

In some aspects, the same active site in the RuvC domain or RuvC-like domain catalyzes the processing of the pre-crRNA and the cleaving of the target nucleic acid. In some aspects, the programmable CasΦ nuclease is fused or linked to one or more NLS. In some aspects, the one or more NLS are fused or linked to the N-terminus of the programmable CasΦ nuclease; the one or more NLS are fused or linked to the C-terminus of the programmable CasΦ nuclease; or the one or more NLS are fused or linked to the N-terminus and the C-terminus of the programmable CasΦ nuclease. In some cases, an aspect comprises the programmable CasΦ nuclease or a nucleic acid described herein and a gRNA comprising a first region that is complementary to a target nucleic acid sequence in a eukaryotic genome and a second region that binds to the programmable CasΦ nuclease.

In some cases, an aspect comprises the programmable CasΦ nuclease or a nucleic acid described herein and a cell, preferably wherein the cell is a eukaryotic cell. In some cases, an aspect comprises the programmable CasΦ nuclease or a nucleic acid described herein and a gRNA comprising a first region that is complementary to a target nucleic acid sequence in a eukaryotic genome and a second region that binds to the programmable CasΦ nuclease and a cell, preferably wherein the cell is a eukaryotic cell. In some cases, an aspect comprises a eukaryotic cell comprising the programmable CasΦ nuclease or a nucleic acid described herein.

In some aspects, the cell further comprises a gRNA comprising a first region that is complementary to a target nucleic acid sequence in a eukaryotic genome and a second region that binds to the programmable CasΦ nuclease and a cell, preferably wherein the cell is a eukaryotic cell.

In some cases, an aspect comprises a vector comprising a nucleic acid described herein. In some aspects, the vector is a viral vector.

In some aspects, the programmable CasΦ nuclease recognizes a protospacer adjacent motif (PAM) of 5′-TTN-3′. In some aspects, the programmable CasΦ nuclease recognizes a protospacer adjacent motif (PAM) of 5′-GTTB-3′, wherein B is C, G, or T. In some aspects, the Cas nuclease recognizes a protospacer adjacent motif (PAM) of 5′-TTN-3′, optionally wherein the PAM is 5′-TTN-3′. In some aspects, the Cas nuclease recognizes a protospacer adjacent motif (PAM) of 5′-GTTK-3′, 5′-VTTK-3′, 5′-VTTS-3′, 5′-TTTS-3′ or 5′-VTTN-3′, where K is G or T, V is A, C or G, and S is C or G. In some aspects, the Cas nuclease recognizes a protospacer adjacent motif (PAM) of 5′-GTTB-3′, wherein B is C, G, or T.

In various aspects, the present disclosure provides a programmable CasΦ nuclease or a nucleic acid encoding said programmable CasΦ nuclease, wherein said programmable CasΦ nuclease comprises at least 85% sequence identity to a sequence selected from the group consisting of SEQ ID NOs: 1 to 47, SEQ ID NO. 105, and SEQ ID NO. 107, and wherein the programmable CasΦ nuclease is capable of binding to a guide RNA comprising a first region that is complementary to a target nucleic acid sequence in a eukaryotic genome and a second region that binds to the programmable CasΦ nuclease; a complex comprising the programmable CasΦ nuclease and the guide RNA binds to the target sequence; the programmable CasΦ nuclease comprises a RuvC domain, wherein the RuvC domain is capable of processing a pre-crRNA and cleaving the target nucleic acid; the programmable CasΦ nuclease cleaves both strands of the target nucleic acid comprising the target sequence, wherein the strand break is a staggered cut with a 5′ overhang; and the programmable CasΦ nuclease does not require a tracrRNA to cleave the target nucleic acid.

In various aspects, the present disclosure provides a programmable CasΦ nuclease or a nucleic acid encoding said programmable CasΦ nuclease, wherein said programmable CasΦ nuclease comprises a RuvC-like domain which matches PFAM family PF07282 and does not match PFAM family PF18516, and wherein the programmable CasΦ nuclease is capable of binding to a guide RNA comprising a first region that is complementary to a target nucleic acid sequence in a eukaryotic genome and a second region that binds to the programmable CasΦ nuclease; a complex comprising the programmable CasΦ nuclease and the guide RNA binds to the target sequence; the RuvC-like domain is capable of processing a pre-crRNA and cleaving the target nucleic acid; the programmable CasΦ nuclease cleaves both strands of the target nucleic acid comprising the target sequence, wherein the strand break is a staggered cut with a 5′ overhang; and the programmable CasΦ nuclease does not require a tracrRNA to cleave the target nucleic acid.

In various aspects, the present disclosure provides a programmable nuclease or a nucleic acid encoding said programmable nuclease, wherein said programmable nuclease is a Type V CRISPR/Cas enzyme nuclease and comprises between 400 and 900 amino acids, and wherein the programmable CasΦ nuclease is capable of binding to a guide RNA comprising a first region that is complementary to a target nucleic acid sequence in a eukaryotic genome and a second region that binds to the programmable CasΦ nuclease; a complex comprising the programmable CasΦ nuclease and the guide RNA binds to the target sequence; the programmable CasΦ nuclease comprises a RuvC domain, wherein the RuvC domain is capable of processing a pre-crRNA and cleaving the target nucleic acid; the programmable CasΦ nuclease cleaves both strands of the target nucleic acid comprising the target sequence, wherein the strand break is a staggered cut with a 5′ overhang; and the programmable CasΦ nuclease does not require a tracrRNA to cleave the target nucleic acid.

In various aspects, the present disclosure provides a programmable CasΦ nuclease or a nucleic acid encoding said programmable CasΦ nuclease, wherein said programmable CasΦ nuclease comprises at least 85% sequence identity to a sequence selected from the group consisting of SEQ ID NOs: 1 to 47, SEQ ID NO. 105, and SEQ ID NO. 107, and wherein the programmable CasΦ nuclease is capable of binding to a guide RNA comprising a first region that is complementary to a target nucleic acid sequence in a eukaryotic genome and a second region that binds to the programmable CasΦ nuclease; a complex comprising the programmable CasΦ nuclease and the guide RNA binds to the target sequence; the programmable CasΦ nuclease comprises a RuvC domain, wherein the RuvC domain is capable of processing a pre-crRNA and cleaving the target nucleic acid; the programmable CasΦ nuclease is capable of cleaving the second region of the guide RNA in mammalian cells; and the programmable CasΦ nuclease does not require a tracrRNA to cleave the target nucleic acid.

In various aspects, the present disclosure provides a programmable CasΦ nuclease or a nucleic acid encoding said programmable CasΦ nuclease, wherein said programmable CasΦ nuclease comprises a RuvC-like domain which matches PFAM family PF07282 and does not match PFAM family PF18516, and wherein the programmable CasΦ nuclease is capable of binding to a guide RNA comprising a first region that is complementary to a target nucleic acid sequence in a eukaryotic genome and a second region that binds to the programmable CasΦ nuclease; a complex comprising the programmable CasΦ nuclease and the guide RNA binds to the target sequence; the RuvC-like domain is capable of processing a pre-crRNA and cleaving the target nucleic acid; the programmable CasΦ nuclease is capable of cleaving the second region of the guide RNA in mammalian cells; and the programmable CasΦ nuclease does not require a tracrRNA to cleave the target nucleic acid.

In various aspects, the present disclosure provides a programmable nuclease or a nucleic acid encoding said programmable nuclease, wherein said programmable nuclease is a Type V CRISPR/Cas enzyme nuclease and comprises between 400 and 900 amino acids, and wherein the programmable CasΦ nuclease is capable of binding to a guide RNA comprising a first region that is complementary to a target nucleic acid sequence in a eukaryotic genome and a second region that binds to the programmable CasΦ nuclease; a complex comprising the programmable CasΦ nuclease and the guide RNA binds to the target sequence; the RuvC-like domain is capable of processing a pre-crRNA and cleaving the target nucleic acid; the programmable CasΦ nuclease is capable of cleaving the second region of the guide RNA in mammalian cells; and the programmable CasΦ nuclease does not require a tracrRNA to cleave the target nucleic acid.

In various aspects, the present disclosure provides a programmable CasΦ nuclease or a nucleic acid encoding said programmable CasΦ nuclease, wherein said programmable CasΦ nuclease comprises at least 85% sequence identity to a sequence selected from the group consisting of SEQ ID NOs: 1 to 47, SEQ ID NO. 105, and SEQ ID NO. 107, and wherein the programmable CasΦ nuclease is capable of binding to a guide RNA comprising a first region that is complementary to a target nucleic acid sequence in a eukaryotic genome and a second region that binds to the programmable CasΦ nuclease; a complex comprising the programmable CasΦ nuclease and the guide RNA binds to the target sequence; the programmable CasΦ nuclease comprises a RuvC domain, wherein the RuvC domain is capable of processing a pre-crRNA and cleaving the target nucleic acid; the programmable CasΦ nuclease cleaves both strands of a target nucleic acid comprising the target sequence, wherein the strand break is a staggered cut with a 5′ overhang; the programmable CasΦ nuclease is capable of cleaving the second region of the guide RNA in mammalian cells; and the programmable CasΦ nuclease does not require a tracrRNA to cleave the target nucleic acid.

In various aspects, the present disclosure provides a programmable CasΦ nuclease or a nucleic acid encoding said programmable CasΦ nuclease, wherein said programmable CasΦ nuclease comprises a RuvC-like domain which matches PFAM family PF07282 and does not match PFAM family PF18516, and wherein the programmable CasΦ nuclease is capable of binding to a guide RNA comprising a first region that is complementary to a target nucleic acid sequence in a eukaryotic genome and a second region that binds to the programmable CasΦ nuclease; a complex comprising the programmable CasΦ nuclease and the guide RNA binds to the target sequence; the RuvC-like domain is capable of processing a pre-crRNA and cleaving the target nucleic acid; the programmable CasΦ nuclease cleaves both strands of a target nucleic acid comprising the target sequence, wherein the strand break is a staggered cut with a 5′ overhang; the programmable CasΦ nuclease is capable of cleaving the second region of the guide RNA in mammalian cells; and the programmable CasΦ nuclease does not require a tracrRNA to cleave the target nucleic acid.

In various aspects, the present disclosure provides a programmable nuclease or a nucleic acid encoding said programmable nuclease, wherein said programmable nuclease is a Type V CRISPR/Cas enzyme nuclease and comprises between 400 and 900 amino acids, and wherein the programmable CasΦ nuclease is capable of binding to a guide RNA comprising a first region that is complementary to a target nucleic acid sequence in a eukaryotic genome and a second region that binds to the programmable CasΦ nuclease; a complex comprising the programmable CasΦ nuclease and the guide RNA binds to the target sequence; the RuvC-like domain is capable of processing a pre-crRNA and cleaving the target nucleic acid; the programmable CasΦ nuclease cleaves both strands of a target nucleic acid comprising the target sequence, wherein the strand break is a staggered cut with a 5′ overhang; the programmable CasΦ nuclease is capable of cleaving the second region of the guide RNA in mammalian cells; and the programmable CasΦ nuclease does not require a tracrRNA to cleave the target nucleic acid.

In various aspects, the present disclosure provides a programmable CasΦ nuclease or a nucleic acid encoding said programmable CasΦ nuclease, wherein said programmable CasΦ nuclease comprises at least 85% sequence identity to a sequence selected from the group consisting of SEQ ID NOs: 1 to 47, SEQ ID NO. 105, and SEQ ID NO. 107, and wherein the programmable CasΦ nuclease is capable of binding to a guide RNA comprising a first region that is complementary to a target nucleic acid sequence in a eukaryotic genome and a second region that binds to the programmable CasΦ nuclease, wherein the first region comprises a seed region comprising between 10 and 16 nucleosides; a complex comprising the programmable CasΦ nuclease and the guide RNA binds to the target sequence; the programmable CasΦ nuclease comprises a RuvC domain, wherein the RuvC domain is capable of processing a pre-crRNA and cleaving the target nucleic acid; and the programmable CasΦ nuclease does not require a tracrRNA to cleave the target nucleic acid.

In various aspects, the present disclosure provides a programmable CasΦ nuclease or a nucleic acid encoding said programmable CasΦ nuclease, wherein said programmable CasΦ nuclease comprises a RuvC-like domain which matches PFAM family PF07282 and does not match PFAM family PF18516, and wherein the programmable CasΦ nuclease is capable of binding to a guide RNA comprising a first region that is complementary to a target nucleic acid sequence in a eukaryotic genome and a second region that binds to the programmable CasΦ nuclease, wherein the first region comprises a seed region comprising between 10 and 16 nucleosides; a complex comprising the programmable CasΦ nuclease and the guide RNA binds to the target sequence; the RuvC-like domain is capable of processing a pre-crRNA and cleaving the target nucleic acid; and the programmable CasΦ nuclease does not require a tracrRNA to cleave the target nucleic acid.

In various aspects, the present disclosure provides a programmable nuclease or a nucleic acid encoding said programmable nuclease, wherein said programmable nuclease is a Type V CRISPR/Cas enzyme nuclease and comprises between 400 and 900 amino acids, and wherein the programmable CasΦ nuclease is capable of binding to a guide RNA comprising a first region that is complementary to a target nucleic acid sequence in a eukaryotic genome and a second region that binds to the programmable CasΦ nuclease, wherein the first region comprises a seed region comprising between 10 and 16 nucleosides; a complex comprising the programmable CasΦ nuclease and the guide RNA binds to the target sequence; the RuvC-like domain is capable of processing a pre-crRNA and cleaving the target nucleic acid; and the programmable CasΦ nuclease does not require a tracrRNA to cleave the target nucleic acid.

In various aspects, the present disclosure provides a programmable CasΦ nuclease or a nucleic acid encoding said programmable CasΦ nuclease, wherein said programmable CasΦ nuclease comprises at least 85% sequence identity to a sequence selected from the group consisting of SEQ ID NOs: 1 to 47, SEQ ID NO. 105, and SEQ ID NO. 107, and wherein the programmable CasΦ nuclease is capable of binding to a guide RNA comprising a first region that is complementary to a target nucleic acid sequence in a eukaryotic genome and a second region that binds to the programmable CasΦ nuclease, wherein the first region comprises a seed region comprising between 10 and 16 nucleosides; a complex comprising the programmable CasΦ nuclease and the guide RNA binds to the target sequence; the programmable CasΦ nuclease comprises a RuvC domain, wherein the RuvC domain is capable of processing a pre-crRNA and cleaving the target nucleic acid; the programmable CasΦ nuclease cleaves both strands of the target nucleic acid comprising the target sequence, wherein the strand break is a staggered cut with a 5′ overhang; and the programmable CasΦ nuclease does not require a tracrRNA to cleave the target nucleic acid.

In various aspects, the present disclosure provides a programmable CasΦ nuclease or a nucleic acid encoding said programmable CasΦ nuclease, wherein said programmable CasΦ nuclease comprises a RuvC-like domain which matches PFAM family PF07282 and does not match PFAM family PF18516, and wherein the programmable CasΦ nuclease is capable of binding to a guide RNA comprising a first region that is complementary to a target nucleic acid sequence in a eukaryotic genome and a second region that binds to the programmable CasΦ nuclease, wherein the first region comprises a seed region comprising between 10 and 16 nucleosides; a complex comprising the programmable CasΦ nuclease and the guide RNA binds to the target sequence; the RuvC-like domain is capable of processing a pre-crRNA and cleaving the target nucleic acid; the programmable CasΦ nuclease cleaves both strands of the target nucleic acid comprising the target sequence, wherein the strand break is a staggered cut with a 5′ overhang; and the programmable CasΦ nuclease does not require a tracrRNA to cleave the target nucleic acid.

In various aspects, the present disclosure provides a programmable nuclease or a nucleic acid encoding said programmable nuclease, wherein said programmable nuclease is a Type V CRISPR/Cas enzyme nuclease and comprises between 400 and 900 amino acids, and wherein the programmable CasΦ nuclease is capable of binding to a guide RNA comprising a first region that is complementary to a target nucleic acid sequence in a eukaryotic genome and a second region that binds to the programmable CasΦ nuclease, wherein the first region comprises a seed region comprising between 10 and 16 nucleosides; a complex comprising the programmable CasΦ nuclease and the guide RNA binds to the target sequence; the RuvC-like domain is capable of processing a pre-crRNA and cleaving the target nucleic acid; the programmable CasΦ nuclease cleaves both strands of the target nucleic acid comprising the target sequence, wherein the strand break is a staggered cut with a 5′ overhang; and the programmable CasΦ nuclease does not require a tracrRNA to cleave the target nucleic acid.

In various aspects, the present disclosure provides a programmable CasΦ nuclease or a nucleic acid encoding said programmable CasΦ nuclease, wherein said programmable CasΦ nuclease comprises at least 85% sequence identity to a sequence selected from the group consisting of SEQ ID NOs: 1 to 47, SEQ ID NO. 105, and SEQ ID NO. 107, and wherein the programmable CasΦ nuclease is capable of binding to a guide RNA comprising a first region that is complementary to a target nucleic acid sequence in a eukaryotic genome and a second region that binds to the programmable CasΦ nuclease, wherein the first region comprises a seed region comprising between 10 and 16 nucleosides; a complex comprising the programmable CasΦ nuclease and the guide RNA binds to the target sequence; the programmable CasΦ nuclease comprises a RuvC domain, wherein the RuvC domain is capable of processing a pre-crRNA and cleaving the target nucleic acid; the programmable CasΦ nuclease is capable of cleaving the second region of the guide RNA in mammalian cells; and the programmable CasΦ nuclease does not require a tracrRNA to cleave the target nucleic acid.

In various aspects, the present disclosure provides a programmable CasΦ nuclease or a nucleic acid encoding said programmable CasΦ nuclease, wherein said programmable CasΦ nuclease comprises a RuvC-like domain which matches PFAM family PF07282 and does not match PFAM family PF18516, and wherein the programmable CasΦ nuclease is capable of binding to a guide RNA comprising a first region that is complementary to a target nucleic acid sequence in a eukaryotic genome and a second region that binds to the programmable CasΦ nuclease, wherein the first region comprises a seed region comprising between 10 and 16 nucleosides; a complex comprising the programmable CasΦ nuclease and the guide RNA binds to the target sequence; the RuvC-like domain is capable of processing a pre-crRNA and cleaving the target nucleic acid; the programmable CasΦ nuclease is capable of cleaving the second region of the guide RNA in mammalian cells; and the programmable CasΦ nuclease does not require a tracrRNA to cleave the target nucleic acid.

In various aspects, the present disclosure provides a programmable nuclease or a nucleic acid encoding said programmable nuclease, wherein said programmable nuclease is a Type V CRISPR/Cas enzyme nuclease and comprises between 400 and 900 amino acids, and wherein the programmable CasΦ nuclease is capable of binding to a guide RNA comprising a first region that is complementary to a target nucleic acid sequence in a eukaryotic genome and a second region that binds to the programmable CasΦ nuclease, wherein the first region comprises a seed region comprising between 10 and 16 nucleosides; a complex comprising the programmable CasΦ nuclease and the guide RNA binds to the target sequence; the RuvC-like domain is capable of processing a pre-crRNA and cleaving the target nucleic acid; the programmable CasΦ nuclease is capable of cleaving the second region of the guide RNA in mammalian cells; and the programmable CasΦ nuclease does not require a tracrRNA to cleave the target nucleic acid.

In various aspects, the present disclosure provides a programmable CasΦ nuclease or a nucleic acid encoding said programmable CasΦ nuclease, wherein said programmable CasΦ nuclease comprises at least 85% sequence identity to a sequence selected from the group consisting of SEQ ID NOs: 1 to 47, SEQ ID NO. 105, and SEQ ID NO. 107, and wherein the programmable CasΦ nuclease is capable of binding to a guide RNA comprising a first region that is complementary to a target nucleic acid sequence in a eukaryotic genome and a second region that binds to the programmable CasΦ nuclease, wherein the first region comprises a seed region comprising between 10 and 16 nucleosides; a complex comprising the programmable CasΦ nuclease and the guide RNA binds to the target sequence; the programmable CasΦ nuclease comprises a RuvC domain, wherein the RuvC domain is capable of processing a pre-crRNA and cleaving the target nucleic acid; the programmable CasΦ nuclease cleaves both strands of a target nucleic acid comprising the target sequence, wherein the strand break is a staggered cut with a 5′ overhang; the programmable CasΦ nuclease is capable of cleaving the second region of the guide RNA in mammalian cells; and the programmable CasΦ nuclease does not require a tracrRNA to cleave the target nucleic acid.

In various aspects, the present disclosure provides a programmable CasΦ nuclease or a nucleic acid encoding said programmable CasΦ nuclease, wherein said programmable CasΦ nuclease comprises a RuvC-like domain which matches PFAM family PF07282 and does not match PFAM family PF18516, and wherein the programmable CasΦ nuclease is capable of binding to a guide RNA comprising a first region that is complementary to a target nucleic acid sequence in a eukaryotic genome and a second region that binds to the programmable CasΦ nuclease, wherein the first region comprises a seed region comprising between 10 and 16 nucleosides; a complex comprising the programmable CasΦ nuclease and the guide RNA binds to the target sequence; the RuvC-like domain is capable of processing a pre-crRNA and cleaving the target nucleic acid; the programmable CasΦ nuclease cleaves both strands of a target nucleic acid comprising the target sequence, wherein the strand break is a staggered cut with a 5′ overhang; the programmable CasΦ nuclease is capable of cleaving the second region of the guide RNA in mammalian cells; and the programmable CasΦ nuclease does not require a tracrRNA to cleave the target nucleic acid.

In various aspects, the present disclosure provides a programmable nuclease or a nucleic acid encoding said programmable nuclease, wherein said programmable nuclease is a Type V CRISPR/Cas enzyme nuclease and comprises between 400 and 900 amino acids, and wherein the programmable CasΦ nuclease is capable of binding to a guide RNA comprising a first region that is complementary to a target nucleic acid sequence in a eukaryotic genome and a second region that binds to the programmable CasΦ nuclease, wherein the first region comprises a seed region comprising between 10 and 16 nucleosides; a complex comprising the programmable CasΦ nuclease and the guide RNA binds to the target sequence; the RuvC-like domain is capable of processing a pre-crRNA and cleaving the target nucleic acid; the programmable CasΦ nuclease cleaves both strands of a target nucleic acid comprising the target sequence, wherein the strand break is a staggered cut with a 5′ overhang; the programmable CasΦ nuclease is capable of cleaving the second region of the guide RNA in mammalian cells; and the programmable CasΦ nuclease does not require a tracrRNA to cleave the target nucleic acid. In some aspects the same active site in the RuvC domain or RuvC-like domain catalyzes the processing of the pre-crRNA and the cleaving of the target nucleic acid.

In some aspects, the programmable CasΦ nuclease is fused or linked to one or more NLS. In some aspects, the one or more NLS are fused or linked to the N-terminus of the programmable CasΦ nuclease; the one or more NLS are fused or linked to the C-terminus of the programmable CasΦ nuclease; or the one or more NLS are fused or linked to the N-terminus and the C-terminus of the programmable CasΦ nuclease.

In some cases, an aspect comprises the programmable CasΦ nuclease or a nucleic acid described herein and a gRNA comprising a first region that is complementary to a target nucleic acid sequence in a eukaryotic genome and a second region that binds to the programmable CasΦ nuclease. In some aspects, the first region comprises a seed region comprising between 10 and 16 nucleosides. In some aspects, the seed region comprises 16 nucleosides. In some cases, an aspect comprises the programmable CasΦ nuclease or a nucleic acid described herein and a cell, preferably wherein the cell is a eukaryotic cell.

In some cases, an aspect comprises the programmable CasΦ nuclease or a nucleic acid described herein and a gRNA comprising a first region that is complementary to a target nucleic acid sequence in a eukaryotic genome and a second region that binds to the programmable CasΦ nuclease and a cell, preferably wherein the cell is a eukaryotic cell. In some aspects, the first region comprises a seed region comprising between 10 and 16 nucleosides. In some aspects, the seed region comprises 16 nucleosides.

In some aspects, a eukaryotic cell comprises the programmable CasΦ nuclease or a nucleic acid described herein. In some aspects, the cell further comprises a gRNA comprising a first region that is complementary to a target nucleic acid sequence in a eukaryotic genome and a second region that binds to the programmable CasΦ nuclease. In some aspects, the first region comprises a seed region comprising between 10 and 16 nucleosides. In some aspects, the seed region comprises 16 nucleosides. In some aspects, a vector comprises a nucleic acid described herein. In some aspects, the vector is a viral vector.

In various aspects, the present disclosure provides a guide nucleic acid, or a nucleic acid encoding said guide nucleic acid, comprising a sequence that is the same as or differs by no more than 5, 4, 3, 2, or 1 nucleotides from: a sequence from Tables A to AH; or a sequence comprising a repeat sequence from Table 2 and a spacer sequence from Tables A to H. In some aspects, the guide nucleic acid comprises a sequence from Tables A to AH; or a sequence comprising a repeat sequence from Table 2 and a spacer sequence from Tables A to H. In some aspects, the guide nucleic acid comprises RNA and/or DNA. In some aspects, the guide nucleic acid is a guide RNA. Some aspects further comprise a complex comprising the guide nucleic acid and a programmable CasΦ nuclease. Some aspects comprise a eukaryotic cell comprising the guide nucleic acid. In some aspects, the eukaryotic cell further comprises a programmable CasΦ nuclease. Some aspects further comprise a vector encoding the guide nucleic acid. In some aspects, the vector is a viral vector.

In various aspects, the present disclosure provides a method of introducing a first modification in a first gene and a second modification in a second gene, the method comprising contacting a cell with a CasΦ nuclease; a first guide RNA that is at least partially complementary to an equal length portion of the first gene; and a second guide RNA that is at least partially complementary to an equal length portion of the second gene. In some aspects, the CasΦ nuclease is a CasΦ12 nuclease. In some aspects, the CasΦ12 nuclease comprises or consists of an amino acid sequence of SEQ ID NO: 12. In some aspects, the first and/or second modification comprises an insertion of a nucleotide, a deletion of a nucleotide or a combination thereof. In some aspects, the first and/or second modification comprises an epigenetic modification. In some aspects, the first and/or second mutation results in a reduction in the expression of the first gene and/or second gene, respectively. In some aspects, the reduction in the expression is at least about a 10% reduction, at least about a 20% reduction, at least about a 30% reduction, at least about a 40% reduction, at least about a 50% reduction, at least about a 60% reduction, at least about a 70% reduction, at least about an 80% reduction, or at least about a 90% reduction. In some aspects, the method comprises contacting the cell with three different guide RNAs targeting three different genes.

In various aspects, the present disclosure provides a programmable CasΦ nuclease or a nucleic acid encoding said programmable CasΦ nuclease, wherein said programmable CasΦ nuclease comprises at least 85% sequence identity to SEQ ID NO: 12. In some aspects, the programmable CasΦ nuclease comprises at least 90% sequence identity to SEQ ID NO: 12. In some aspects, the programmable CasΦ nuclease comprises at least 95% sequence identity to SEQ ID NO: 12. In some aspects, the programmable CasΦ nuclease comprises at least 98% sequence identity to SEQ ID NO: 12. In some aspects, the programmable CasΦ nuclease comprises or consists of an amino acid sequence of SEQ ID NO: 12. In some aspects, the programmable CasΦ nuclease comprises at least 85% sequence identity to SEQ ID NO: 18. In some aspects, the programmable CasΦ nuclease comprises at least 90% sequence identity to SEQ ID NO: 18. In some aspects, the programmable CasΦ nuclease comprises at least 95% sequence identity to SEQ ID NO: 18. In some aspects, the programmable CasΦ nuclease comprises at least 98% sequence identity to SEQ ID NO: 18. In some aspects, the programmable CasΦ nuclease comprises or consists of an amino acid sequence of SEQ ID NO: 18. In some aspects, the programmable CasΦ nuclease comprises at least 85% sequence identity to SEQ ID NO: 32. In some aspects, the programmable CasΦ nuclease comprises at least 85% sequence identity to SEQ ID NO: 32. In some aspects, the programmable CasΦ nuclease comprises at least 90% sequence identity to SEQ ID NO: 32. In some aspects, the programmable CasΦ nuclease comprises at least 95% sequence identity to SEQ ID NO: 32. In some aspects, the programmable CasΦ nuclease comprises at least 98% sequence identity to SEQ ID NO: 32. In some aspects, the programmable CasΦ nuclease comprises or consists of an amino acid sequence of SEQ ID NO: 32. In some aspects, the programmable CasΦ nuclease is capable of binding to a guide RNA comprising a first region that is complementary to a target nucleic acid sequence in a eukaryotic genome and a second region that binds to the programmable CasΦ nuclease. In some aspects, the a complex comprising the programmable CasΦ nuclease and the guide RNA binds to the target sequence. In some aspects, the programmable CasΦ nuclease does not require a tracrRNA to cleave a target nucleic acid. In some aspects, the programmable CasΦ nuclease comprises a RuvC domain, wherein the RuvC domain is capable of processing a pre-crRNA and cleaving a target nucleic acid.

In various aspects, the present disclosure provides a composition comprising the programmable CasΦ nuclease disclosed herein or a nucleic acid encoding said programmable nuclease, and a guide nucleic acid comprising a first region that is complementary to a target nucleic acid sequence in a eukaryotic genome and a second region that binds to the programmable CasΦ nuclease. In some aspects, the first region comprises a seed region comprising between 10 and 16 nucleosides. In some aspects, the seed region comprises 16 nucleosides. In some aspects, the composition comprises the programmable CasΦ nuclease or a nucleic acid encoding said programmable nuclease and a cell, preferably wherein the cell is a eukaryotic cell. In various aspects, the present disclosure provides a programmable CasΦ nuclease disclosed herein or a nucleic acid encoding said programmable nuclease, and a guide nucleic acid comprising a first region that is complementary to a target nucleic acid sequence in a eukaryotic genome and a second region that binds to the programmable CasΦ nuclease and a cell, preferably wherein the cell is a eukaryotic cell. In some aspects, the first region comprises a seed region comprising between 10 and 16 nucleosides. In some aspects, the seed region comprises 16 nucleosides.

In various aspects, the present disclosure provides a eukaryotic cell comprising the programmable CasΦ nuclease disclosed herein or a nucleic acid encoding said programmable nuclease. In some aspects, the cell further comprises a guide nucleic acid comprising a first region that is complementary to a target nucleic acid sequence in a eukaryotic genome and a second region that binds to the programmable CasΦ nuclease. In some aspects, the first region comprises a seed region comprising between 10 and 16 nucleosides. In some aspects, the seed region comprises 16 nucleosides.

In various aspects, the present disclosure provides a vector comprising the nucleic acid encoding a programmable nuclease as disclosed herein. In some aspects, the vector is a viral vector. In some aspects, the vector further comprises a nucleic acid encoding a guide nucleic acid, wherein the guide nucleic acid comprises a first region that is complementary to a target nucleic acid sequence in a eukaryotic genome and a second region that binds to the programmable CasΦ nuclease. In some aspects, the guide nucleic acid is a guide RNA. In some aspects, the vector further comprises a donor polynucleotide. In some aspects, the guide nucleic acid is a guide RNA.

In various aspects, the present disclosure provides a programmable nuclease or a nucleic acid encoding said programmable nuclease, wherein said programmable nuclease is a Type V CRISPR/Cas enzyme nuclease and comprises between 400 and 900 amino acids, and wherein the programmable nuclease is capable of binding to a guide RNA comprising a first region that is complementary to a target nucleic acid sequence in a eukaryotic genome and a second region that binds to the programmable nuclease; a complex comprising the programmable nuclease and the guide RNA binds to the target sequence; the programmable nuclease comprises a RuvC domain, wherein the RuvC domain is capable of processing a pre-crRNA and cleaving the target nucleic acid; the programmable nuclease cleaves both strands of the target nucleic acid comprising the target sequence, wherein the strand break is a staggered cut with a 5′ overhang; and the programmable nuclease does not require a tracrRNA to cleave the target nucleic acid.

In various aspects, the present disclosure provides a programmable nuclease or a nucleic acid encoding said programmable nuclease, wherein said programmable nuclease is a Type V CRISPR/Cas enzyme nuclease and comprises between 400 and 900 amino acids, and wherein the programmable nuclease is capable of binding to a guide RNA comprising a first region that is complementary to a target nucleic acid sequence in a eukaryotic genome and a second region that binds to the programmable nuclease; a complex comprising the programmable nuclease and the guide RNA binds to the target sequence; the RuvC-like domain is capable of processing a pre-crRNA and cleaving the target nucleic acid; the programmable nuclease is capable of cleaving the second region of the guide RNA in mammalian cells; and the programmable nuclease does not require a tracrRNA to cleave the target nucleic acid.

In various aspects, the present disclosure provides a programmable nuclease or a nucleic acid encoding said programmable nuclease, wherein said programmable nuclease is a Type V CRISPR/Cas enzyme nuclease and comprises between 400 and 900 amino acids, and wherein the programmable nuclease is capable of binding to a guide RNA comprising a first region that is complementary to a target nucleic acid sequence in a eukaryotic genome and a second region that binds to the programmable nuclease; a complex comprising the programmable nuclease and the guide RNA binds to the target sequence; the RuvC-like domain is capable of processing a pre-crRNA and cleaving the target nucleic acid; the programmable nuclease cleaves both strands of a target nucleic acid comprising the target sequence, wherein the strand break is a staggered cut with a 5′ overhang; the programmable nuclease is capable of cleaving the second region of the guide RNA in mammalian cells; and the programmable nuclease does not require a tracrRNA to cleave the target nucleic acid.

In various aspects, the present disclosure provides a programmable nuclease or a nucleic acid encoding said programmable nuclease, wherein said programmable nuclease is a Type V CRISPR/Cas enzyme nuclease and comprises between 400 and 900 amino acids, and wherein the programmable nuclease is capable of binding to a guide RNA comprising a first region that is complementary to a target nucleic acid sequence in a eukaryotic genome and a second region that binds to the programmable nuclease, wherein the first region comprises a seed region comprising between 10 and 16 nucleosides; a complex comprising the programmable nuclease and the guide RNA binds to the target sequence; the RuvC-like domain is capable of processing a pre-crRNA and cleaving the target nucleic acid; and the programmable nuclease does not require a tracrRNA to cleave the target nucleic acid.

In various aspects, the present disclosure provides a programmable nuclease or a nucleic acid encoding said programmable nuclease, wherein said programmable nuclease is a Type V CRISPR/Cas enzyme nuclease and comprises between 400 and 900 amino acids, and wherein the programmable nuclease is capable of binding to a guide RNA comprising a first region that is complementary to a target nucleic acid sequence in a eukaryotic genome and a second region that binds to the programmable nuclease, wherein the first region comprises a seed region comprising between 10 and 16 nucleosides; a complex comprising the programmable nuclease and the guide RNA binds to the target sequence; the RuvC-like domain is capable of processing a pre-crRNA and cleaving the target nucleic acid; the programmable nuclease cleaves both strands of the target nucleic acid comprising the target sequence, wherein the strand break is a staggered cut with a 5′ overhang; and the programmable nuclease does not require a tracrRNA to cleave the target nucleic acid.

In various aspects, the present disclosure provides a programmable nuclease or a nucleic acid encoding said programmable nuclease, wherein said programmable nuclease is a Type V CRISPR/Cas enzyme nuclease and comprises between 400 and 900 amino acids, and wherein the programmable nuclease is capable of binding to a guide RNA comprising a first region that is complementary to a target nucleic acid sequence in a eukaryotic genome and a second region that binds to the programmable nuclease, wherein the first region comprises a seed region comprising between 10 and 16 nucleosides; a complex comprising the programmable nuclease and the guide RNA binds to the target sequence; the RuvC-like domain is capable of processing a pre-crRNA and cleaving the target nucleic acid; the programmable nuclease is capable of cleaving the second region of the guide RNA in mammalian cells; and the programmable nuclease does not require a tracrRNA to cleave the target nucleic acid.

In various aspects, the present disclosure provides a programmable nuclease or a nucleic acid encoding said programmable nuclease, wherein said programmable nuclease is a Type V CRISPR/Cas enzyme nuclease and comprises between 400 and 900 amino acids, and wherein the programmable nuclease is capable of binding to a guide RNA comprising a first region that is complementary to a target nucleic acid sequence in a eukaryotic genome and a second region that binds to the programmable nuclease, wherein the first region comprises a seed region comprising between 10 and 16 nucleosides; a complex comprising the programmable nuclease and the guide RNA binds to the target sequence; the RuvC-like domain is capable of processing a pre-crRNA and cleaving the target nucleic acid; the programmable nuclease cleaves both strands of a target nucleic acid comprising the target sequence, wherein the strand break is a staggered cut with a 5′ overhang; the programmable nuclease is capable of cleaving the second region of the guide RNA in mammalian cells; and the programmable nuclease does not require a tracrRNA to cleave the target nucleic acid. In some aspects, the same active site in the RuvC domain or RuvC-like domain catalyzes the processing of the pre-crRNA and the cleaving of the target nucleic acid. In some aspects, the programmable nuclease is fused or linked to one or more NLS.

In various aspects, the programmable nuclease disclosed herein or the nucleic acid encoding said programmable nuclease is fused to one or more NLS. In some aspects, the one or more NLS are fused or linked to the N-terminus of the programmable nuclease. In some aspects, the one or more NLS are fused or linked to the C-terminus of the programmable nuclease; or the one or more NLS are fused or linked to the N-terminus and the C-terminus of the programmable nuclease.

In various aspects, the present disclosure provides a composition comprising a programmable nuclease disclosed herein or a nucleic acid encoding the programmable nuclease; and a gRNA comprising a first region that is complementary to a target nucleic acid sequence in a eukaryotic genome and a second region that binds to the programmable nuclease. In some aspects, the first region comprises a seed region comprising between 10 and 16 nucleosides. In some aspects, the seed region comprises 16 nucleosides. In some aspects, the programmable nuclease or a nucleic acid disclosed herein is comprised in a cell, preferably wherein the cell is a eukaryotic cell. In some aspects, the composition comprising the programmable nuclease or a nucleic acid disclosed herein further comprises a gRNA comprising a first region that is complementary to a target nucleic acid sequence in a eukaryotic genome and a second region that binds to the programmable nuclease and a cell, preferably wherein the cell is a eukaryotic cell. In some aspects, the first region comprises a seed region comprising between 10 and 16 nucleosides. In some aspects, the seed region comprises 16 nucleosides.

In various aspects, the present disclosure provides a eukaryotic cell comprising a programmable nuclease disclosed herein or a nucleic acid molecule encoding said programmable nuclease. In some aspects, the cell further comprises a gRNA comprising a first region that is complementary to a target nucleic acid sequence in a eukaryotic genome and a second region that binds to the programmable nuclease. In some aspects, the first region comprises a seed region comprising between 10 and 16 nucleosides. In some aspects, the seed region comprises 16 nucleosides. In some aspects, the nucleic acid disclosed herein is comprised in a vector. In some aspects, the vector is a viral vector.

In some aspects, the present disclosure provides a complex comprising a first programmable CasΦ nuclease and a second programmable CasΦ nuclease. In some aspects, the first programmable CasΦ nuclease and the second programmable CasΦ nuclease are the same programmable CasΦ nuclease. In some aspects, the dimer comprises a first programmable CasΦ nuclease and a second programmable CasΦ nuclease. In some aspects, the composition comprises a first programmable CasΦ nuclease and a second programmable CasΦ nuclease.

In various aspects, the present disclosure provides a method of modifying a cell comprising a target nucleic acid, comprising introducing a composition comprising a programmable CasΦ nuclease, programmable nuclease or a cas nuclease to a cell, wherein the programmable CasΦ nuclease, programmable nuclease or the cas nuclease cleaves the target nucleic acid, thereby modifying the cell.

In various aspects, the disclosure provides a method of modifying a cell comprising a target nucleic acid, comprising introducing to the cell (i) the programmable CasΦ nuclease or programmable nuclease disclosed herein and (ii) a guide nucleic acid, wherein the programmable CasΦ nuclease or programmable Cas nuclease cleaves the target nucleic acid, thereby modifying the cell. In some aspects, the guide nucleic acid is a guide RNA. In some aspects, the method further comprises introducing a donor polynucleotide to the cell. In some aspects, the method comprises inserting the donor polynucleotide into the target nucleic acid at the site of cleavage. In some aspects, the cell is a eukaryotic cell, preferably a human cell. In some aspects, the cell is a T cell. In some aspects, the cell is a CAR-T cell. In some aspects, the cell is a stem cell. In some aspects, the cell is a hematopoietic stem cell. In some aspects, the stem cell is a pluripotent stem cell, preferably an induced pluripotent stem cell. In some aspects, the modified cell obtained or obtainable by the method disclosed herein. In some aspect, the disclosure provides a modified human cell obtained or obtainable by the methods herein. In some aspects, the modified cell is a eukaryotic cell, preferably a human cell. In some aspects, the cell is a T cell. In some aspects, the T cell is a CAR-T cell. In some aspects, the cell is a stem cell. In some aspects, the cell is a hematopoietic stem cell. In some aspects, the cell is a pluripotent stem cell, preferably an induced pluripotent stem cell.

In some aspects, the method comprises the use of a CasΦ nuclease to introduce a first modification in a first gene and a second modification in a gene according to the methods disclosed herein. In some aspects, the method comprises the use of a programmable CasΦ nuclease, programmable nuclease or a cas nuclease to modify a cell according to the methods disclosed herein. In some aspects, the method comprises lipid nanoparticle delivery of a nucleic acid encoding the programmable CasΦ nuclease, programmable nuclease or cas nuclease, and the guide nucleic acid. In some aspects, the nucleic acid further comprises a donor polynucleotide. In some aspects, the nucleic acid is a viral vector. In some aspects, the viral vector is an AAV vector.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

42256-779_601_SL The patent or application file contains at least one drawing executed in color. Copies of this patent or patent application publication with color drawing(s) will be provided by the Office upon request and payment of the necessary fee.

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

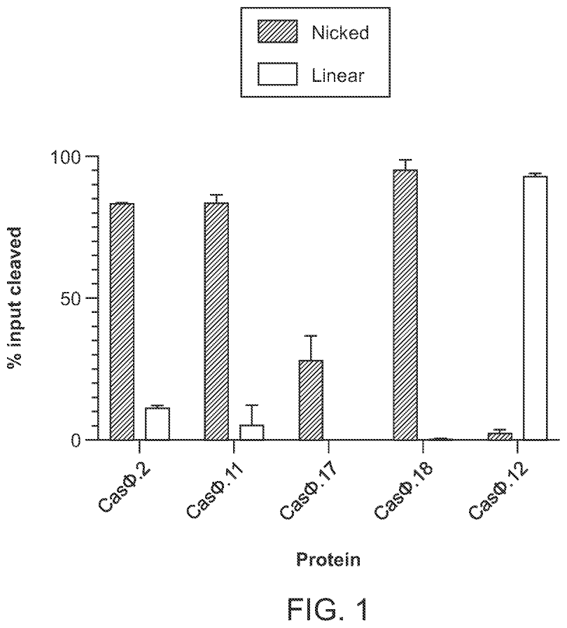

FIG. 1 illustrates results of a cis-cleavage assay on CasΦ polypeptides to assess programmable nickase activity. The results showed that CasΦ orthologs comprise programmable nickase activity. The assay was performed on five CasΦ polypeptides, designated CasΦ.2, CasΦ.11, CasΦ.17, CasΦ.18, and CasΦ.12, in FIG. 1. For the assay, each of the CasΦ polypeptides was complexed with a guide nucleic acid at room temperature for 20 minutes to form a ribonucleoprotein (RNP) complex. The RNP complexes for each of the CasΦ polypeptides were separately incubated at 37° C. for 60 minutes with plasmid DNA targeted by the guide nucleic acids. The graph shows the percentage of plasmids that developed nicks (single-stranded breaks) or linearized (double-stranded breaks) during the 60 minute incubation, as measured by gel-electrophoresis. The data showed that CasΦ.2, CasΦ.11, CasΦ.17, and CasΦ.18 acted as programmable nickases. CasΦ.17 and CasΦ.18 produced only nicked product. CasΦ.2 and CasΦ.11 generated some linearized product but primarily nicked intermediate. CasΦ.12 generated almost entirely linearized product.

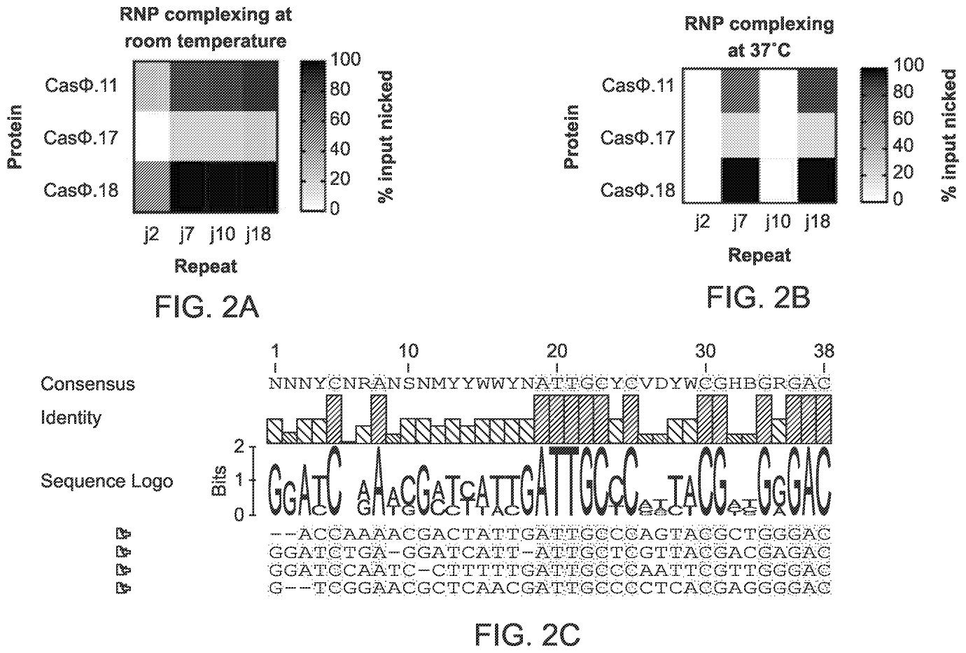

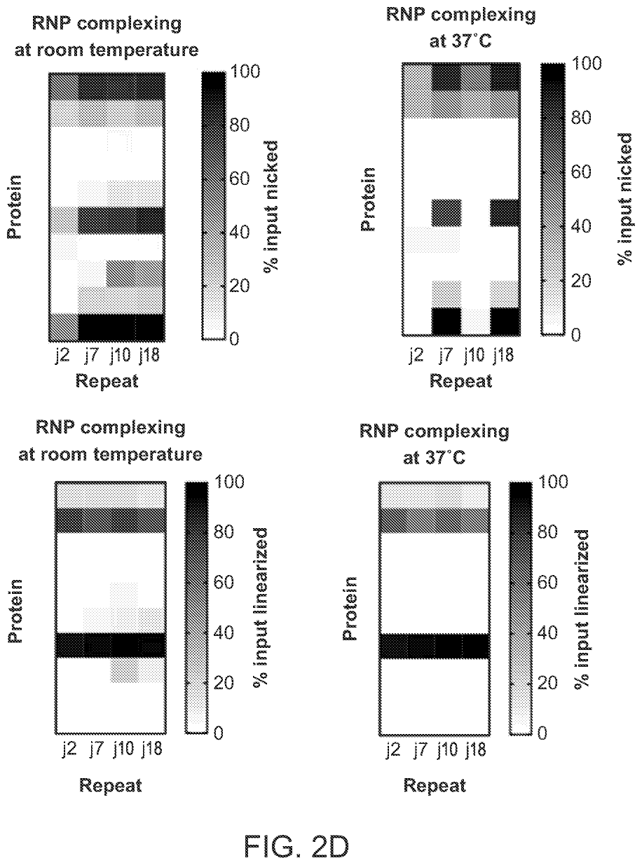

FIG. 2A and FIG. 2B illustrate results of a cis-cleavage assay on CasΦ polypeptides to assess the effect of crRNA repeat sequence and RNP complexing temperature on the programmable nickase activity of CasΦ polypeptides. Each of three proteins (designated CasΦ.11, CasΦ.17 and CasΦ.18 in FIG. 2A and FIG. 2B) was tested for its ability to nick plasmid DNA when complexed with one of four crRNAs comprising the repeat sequences of CasΦ.2, CasΦ.7, CasΦ.10 and CasΦ.18 (abbreviated j2, j7, j10, and j18, respectively, in FIG. 2A and FIG. 2B). FIG. 2C illustrates the alignment of CasΦ.2, CasΦ.7, CasΦ.10, and CasΦ.18 repeat sequences showing conserved (highlighted in black) and diverged nucleotides. For the assay, the RNP complex formation of each of the CasΦ polypeptides with the guide nucleic acid was performed at either room temperature or at 37° C. The incubation of the RNP complex with the input plasmid DNA that comprised the target sequence for the guide nucleic acids was carried out for 60 minutes at 37° C. FIG. 2A shows the percentage of input plasmid DNA that was nicked by RNP complexes assembled at room temperature. The data showed that crRNAs comprising repeat sequences from all tested CasΦ polypeptides supported nickase activity by CasΦ.11, CasΦ.17, and CasΦ.18; the only exception was the CasΦ.17/CasΦ.2-repeat pairing. FIG. 2B shows the percentage of input plasmid DNA that was nicked by RNP complexes assembled at 37° C. The data showed that the activity of each protein is completely abolished when complexed with crRNAs comprising a repeat sequence from CasΦ.2 or CasΦ.10. FIG. 2D shows corresponding data for CasΦ.2, CasΦ.4, CasΦ.6, CasΦ.9, CasΦ.10, CasΦ.12 and CasΦ.13 for the experiment shown in FIG. 2A and FIG. 2B. FIG. 2D also shows the percentage of input plasmid DNA that was linearized by CasΦ.2, CasΦ.4, CasΦ.6, CasΦ.9, CasΦ.10, CasΦ.11, CasΦ.12, CasΦ.13, CasΦ.17 and CasΦ.18 when complexed with one of four crRNAs J2, j7, j10 and j18, as described above.

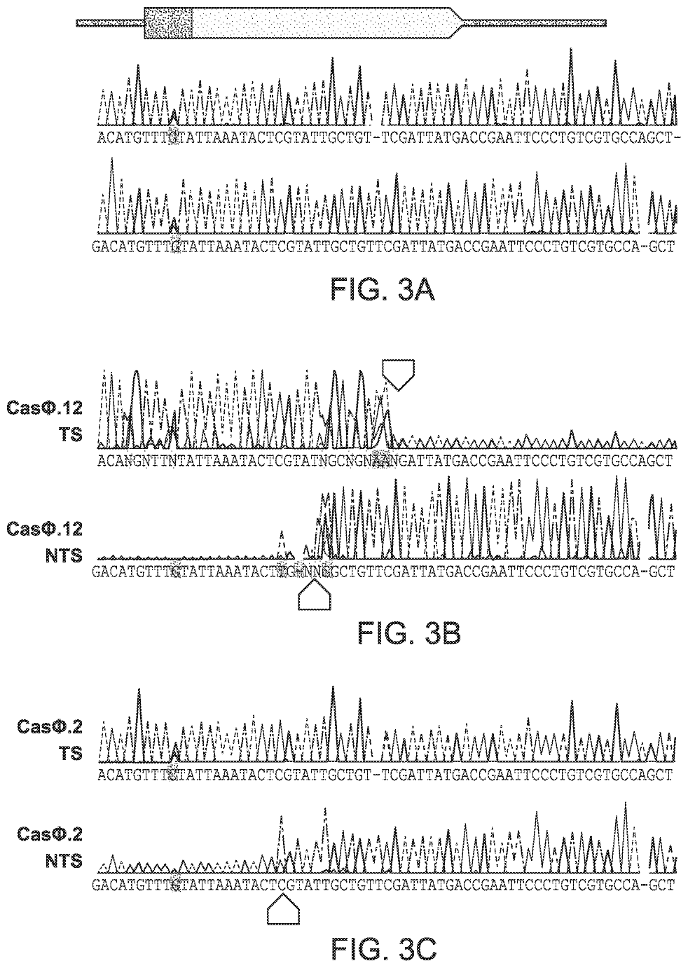

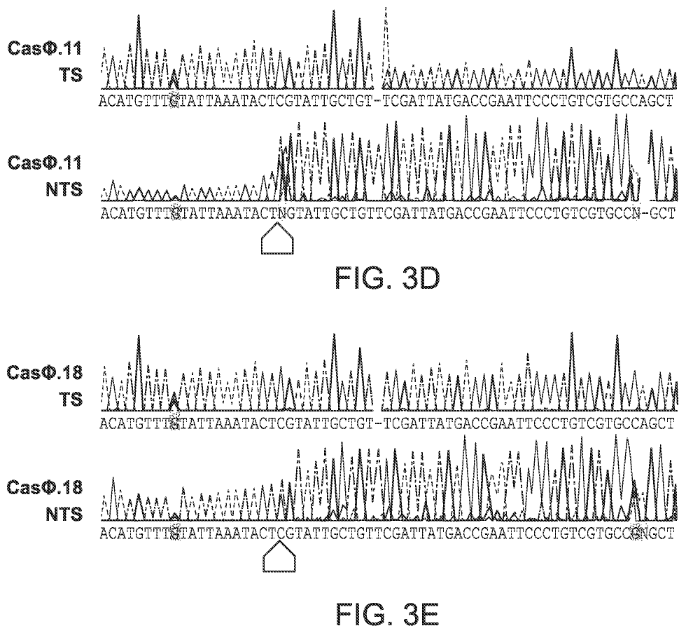

FIG. 3 illustrates results of a cis-cleavage assay and sequencing run demonstrating that CasΦ nickases cleave the non-target strand of a double-stranded DNA target. A cis-cleavage assay was performed with four CasΦ polypeptides, CasΦ.12, CasΦ.2, CasΦ.11, and CasΦ.18, and a control comprising no CasΦ polypeptide, on a super-coiled plasmid DNA comprising a protospacer immediately downstream of a TTTN PAM sequence. The resulting DNA from the assay was Sanger sequenced using forward and reverse primers. The forward primer comprised the sequence of the target strand (TS) of the DNA sequence, while the reverse primer comprised the sequence of the non-target strand (NTS). If a strand had been cleaved by the CasΦ polypeptide being assayed, the sequencing signal would drop off from the cleavage site. FIG. 3A illustrates the cleavage pattern for the control that comprised no CasΦ polypeptide. In the absence of CasΦ polypeptide, the target DNA remained uncut and resulted in complete sequencing of both target and non-target strands. FIG. 3B illustrates the cleavage pattern for CasΦ.12 protein, which comprises double-stranded DNA cleavage activity. As shown in the figure, the sequencing signal dropped off on both the target and the non-target strands (as shown by arrows) demonstrating cleavage of both strands. FIG. 3C illustrates the cleavage pattern for CasΦ.2, which predominantly nicks DNA as illustrated in FIG. 1. The sequencing signal dropped off only on the non-target strand (bottom arrow) demonstrating nicking of the non-target strand. FIG. 3D illustrates the cleavage pattern for CasΦ.11. As illustrated in FIG. 1, CasΦ.11 only nicks DNA after 60 minutes of incubation with plasmid DNA. The sequencing signal dropped off on the non-target strand (bottom arrow), thus demonstrating that CasΦ.11 nicks the non-target strand. FIG. 3E illustrates the cleavage pattern for CasΦ.18. As illustrated in FIG. 1, CasΦ.18 only nicks DNA after 60 minutes of incubation with plasmid DNA. The sequencing signal dropped off on the non-target strand (bottom arrow), thus demonstrating that CasΦ.18 nicks the non-target strand.

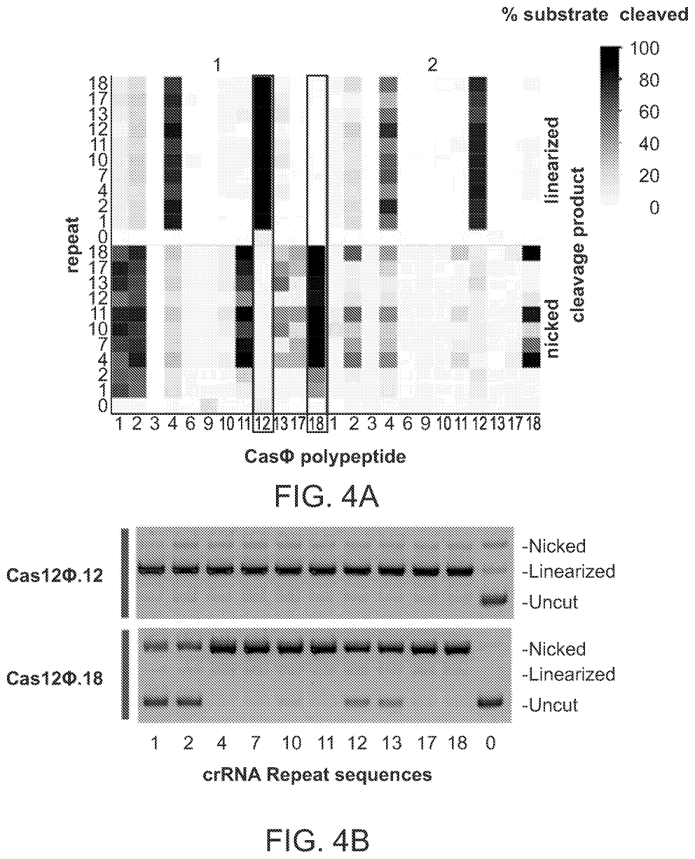

FIG. 4 illustrates results of a cis-cleavage assay on CasΦ polypeptides to assess the effect of crRNA repeat and target sequence the programmable nickase and double strand DNA cleavage activity of CasΦ polypeptides. The heat map in FIG. 4A cleavage products for 60 minute in vitro plasmid cleavage reactions of 12 CasΦ orthologs paired with 10 crRNA repeat sequences. Except for 0, all Repeat and CasΦ axis labels refer Cas12Φ system numbers. Repeat 0 is a negative control including the CasΦ.18 crRNA repeat sequence and a non-targeting spacer sequence. With rare exceptions, preference for nicking or linearizing target DNA is not affected by crRNA repeat or target DNA sequence. Raw data for CasΦ.12 and CasΦ.18 targeting spacer 1 (boxes) are shown in B. FIG. 4B shows the raw gel data used to generate a subset of the heat map from FIG. 4A. CasΦ.12 predominantly linearizes plasmid DNA (i.e. cleaves both strands of a double strand DNA target) whereas CasΦ.18 primarily does not proceed beyond the first strand nicking.

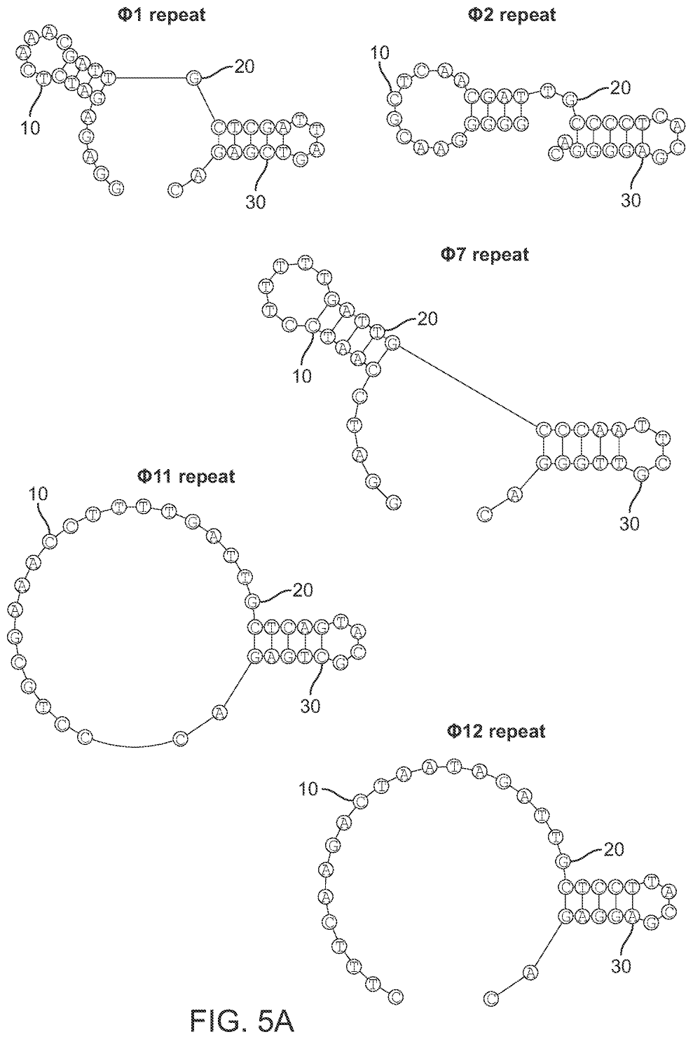

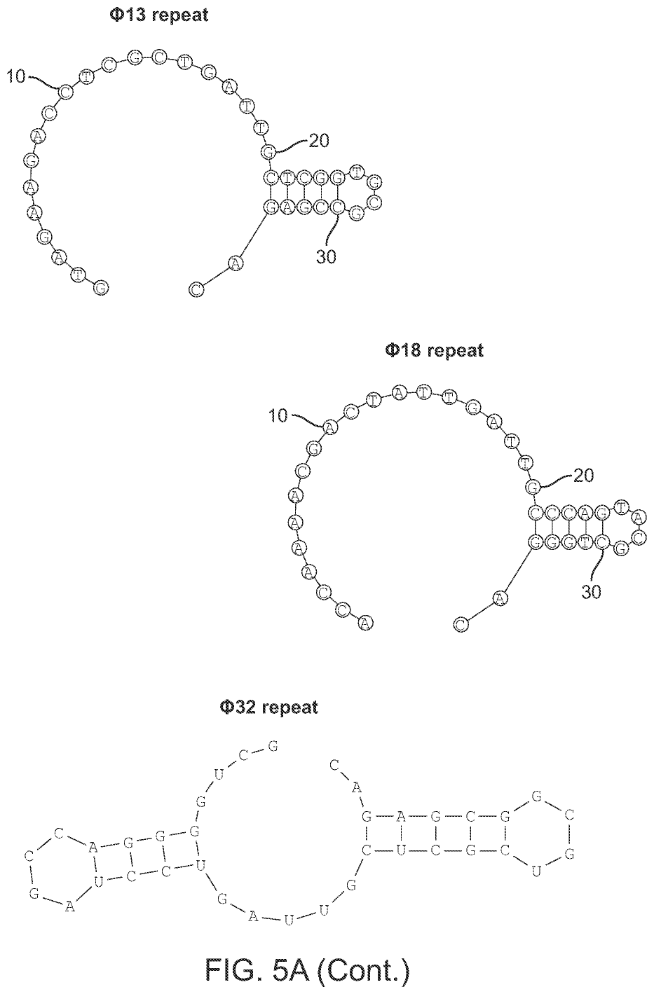

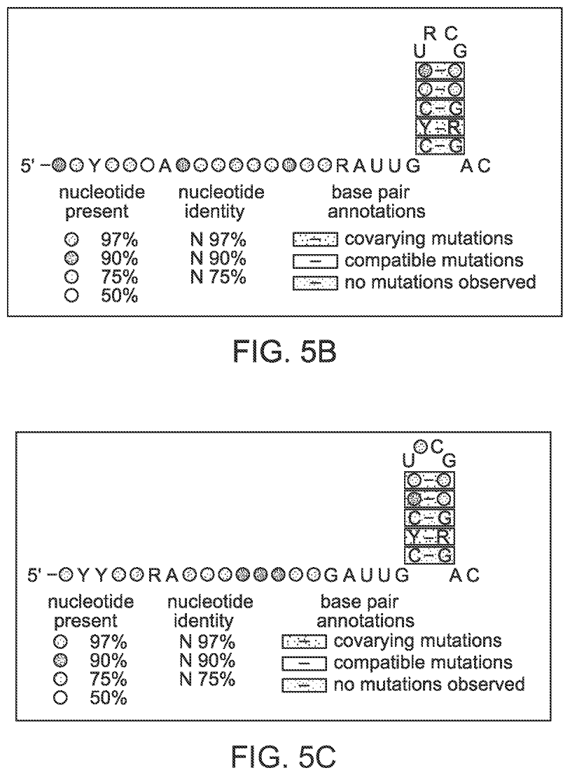

FIG. 5 illustrates the structural conservation of CasΦ crRNA repeats. FIG. 5A shows the structure of the crRNA repeats for CasΦ.1, CasΦ.2, CasΦ.7, CasΦ.11, CasΦ.12, CasΦ.13, CasΦ.18, and CasΦ.32. These structures were calculated using an online RNA prediction tool (https://rna.urmc.rochester.edu/RNAstructureWeb/Servers/Predictl/Predictl.html) using default parameters at 37° C. The sequences of these repeats are provided in TABLE 2. FIG. 5B shows the consensus structure of the crRNA as determined by the LocaRNA tool using the crRNA repeats from CasΦ.1, CasΦ.2, CasΦ.4, CasΦ.7, CasΦ.10, CasΦ.11, CasΦ.12, CasΦ.13, Cas120.17, CasΦ.18, CasΦ.19, CasΦ.21, CasΦ.22, CasΦ.23, CasΦ.24, CasΦ.25, CasΦ.26, CasΦ.27, CasΦ.28, CasΦ.29, CasΦ.30, CasΦ.31, CasΦ.32, CasΦ.33, CasΦ.35 and CasΦ.41. FIG. 5C shows a further refined consensus structure of the crRNA determined by the LocaRNA tool. The LocaRNA tool aligns RNA sequences while considering consensus secondary structure of the RNA sequence.

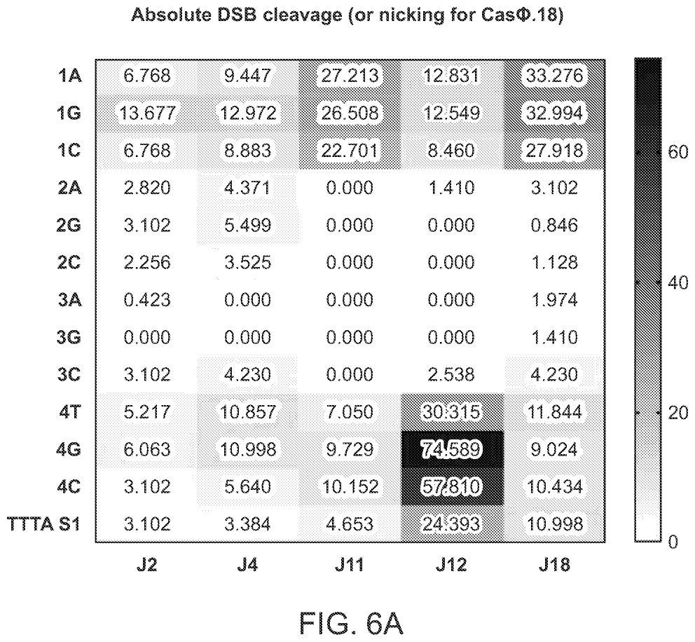

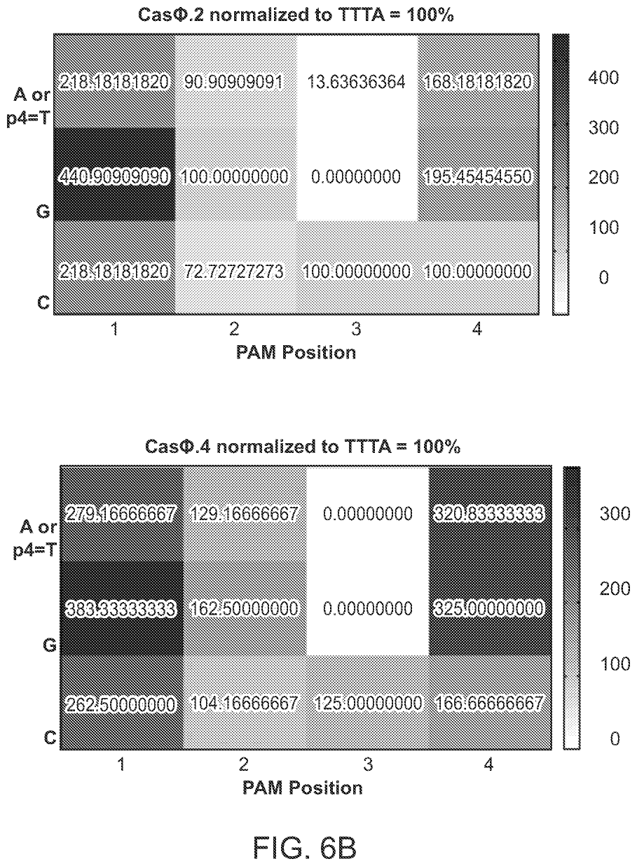

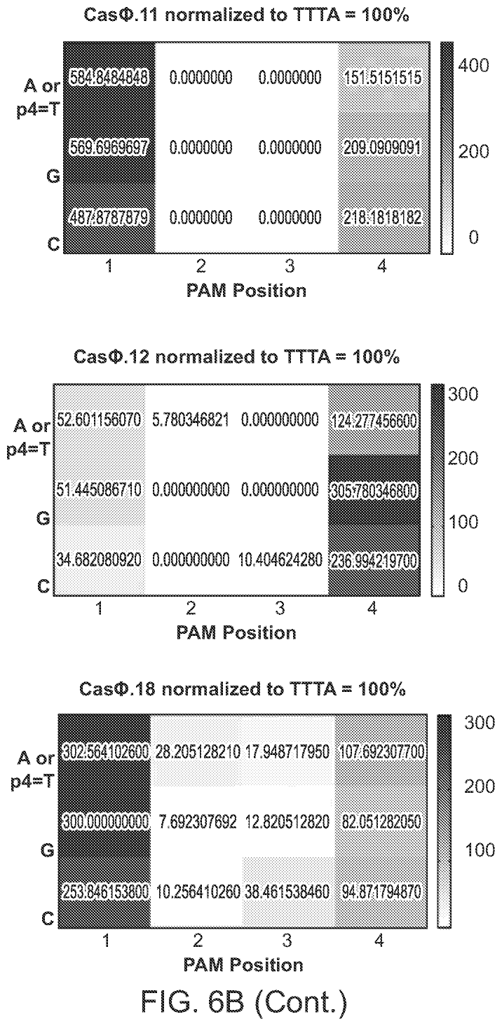

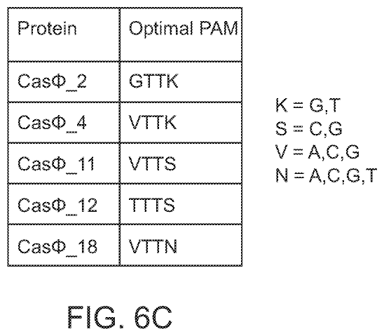

FIG. 6 illustrates the optimal PAM preferences for CasΦ.2, CasΦ.4, CasΦ.11, CasΦ.12 and CasΦ.18. An in vitro cleavage assay was performed using a linear DNA target. Starting with a TTTA PAM, each position was varied one by one to the other 3 nucleotides for a total of 12 variants in addition to parental TTTA. FIG. 6A shows a heat map which illustrates the absolute levels of double strand cleavage (or nicking for CasΦ.18). FIG. 6B shows the data from FIG. 6A after normalization to the parental TTTA PAM as 100%. FIG. 6C shows the optimal PAM preferences of these CasΦ polypeptides with a summary of the data shown in FIG. 6A and FIG. 6B.

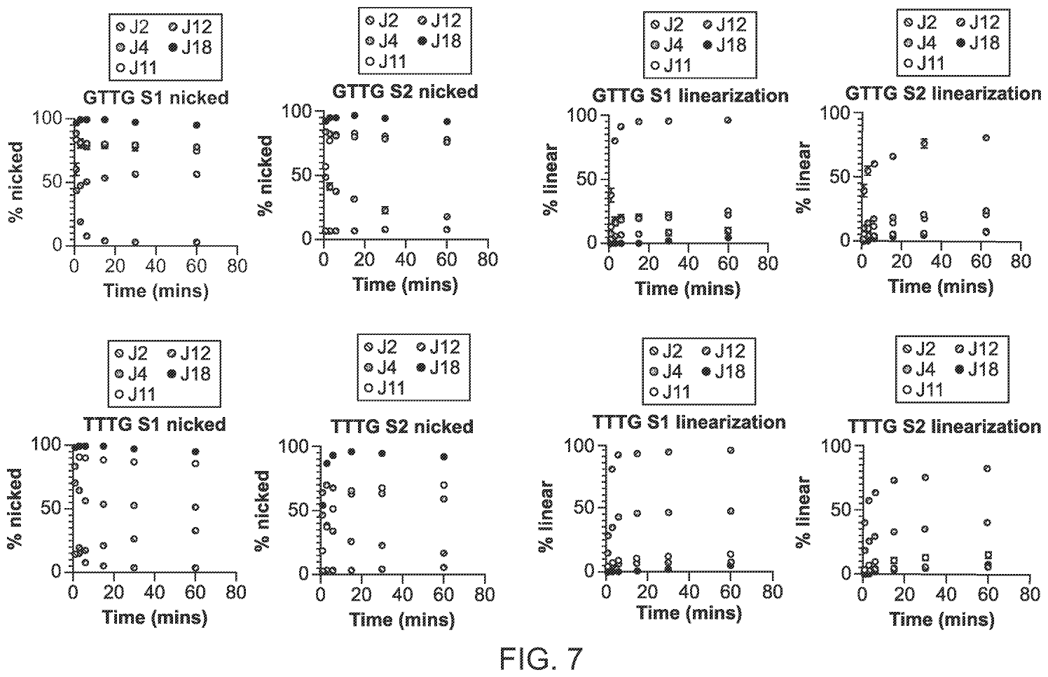

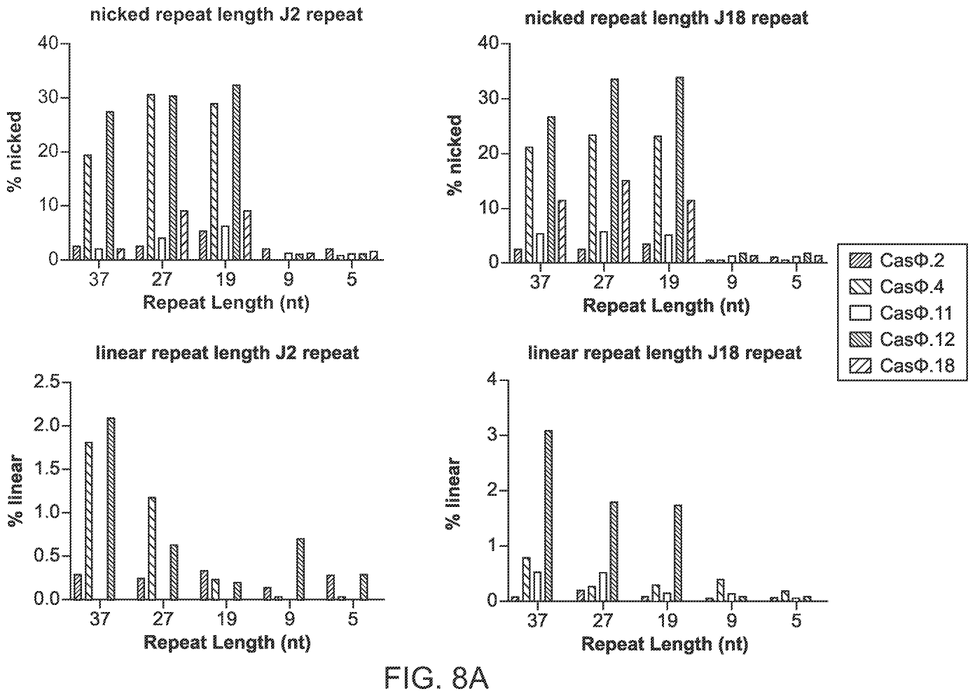

FIG. 7 illustrates that CasΦ polypeptides rapidly nick supercoiled DNA. CasΦ polypeptides where assembled with their native repeat crRNAs targeting one of two targets (S1, TATTAAATACTCGTATTGCTGTTCGATTAT (SEQ ID NO: 108), or S2, CACAGCTTGTCTGTAAGCGGATGCCATATG (SEQ ID NO: 109)) immediately downstream of a GTTG or TTTG PAM. Reactions were initiated with the addition of supercoiled target DNA and stopped after 1, 3, 6, 15, 30 and 60 mins. The cleavage was quantified by agarose gel analysis as nicked (left column) or linear (right column). Error bars are +/− SEM of duplicate time courses.

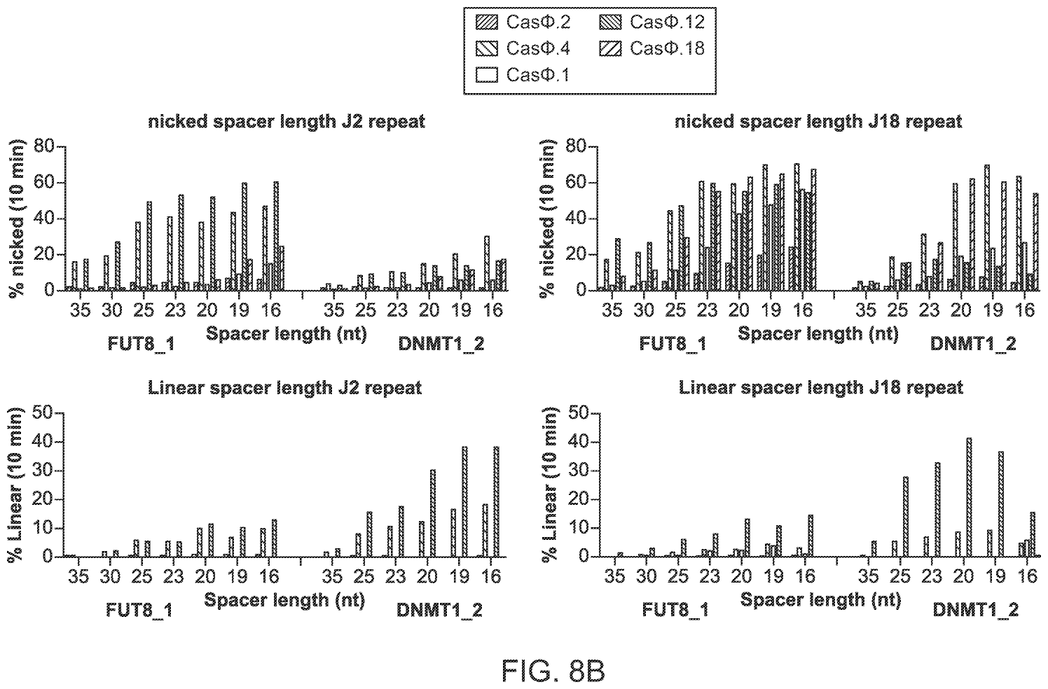

FIG. 8 illustrates that CasΦ polypeptides prefer full-length repeats and spacers from 16 to 20 nucleotides. crRNA panels varying in repeat and spacer length were tested for their ability to support CasΦ polypeptides spacer cleavage. Two different CasΦ repeats that function across CasΦ orthologs were utilized. FIG. 8A shows results of the assay for nicking (top) or linearization (bottom) as influenced by the length of the crRNA repeat. 19 nucleotides was the shortest repeat still supporting cleaving activity. FIG. 8B shows results for nicking (top) or linearization (bottom) as influenced by the length of the crRNA spacer. The optimal spacer length varied by target but is generally 16 to 20 nucleotides.

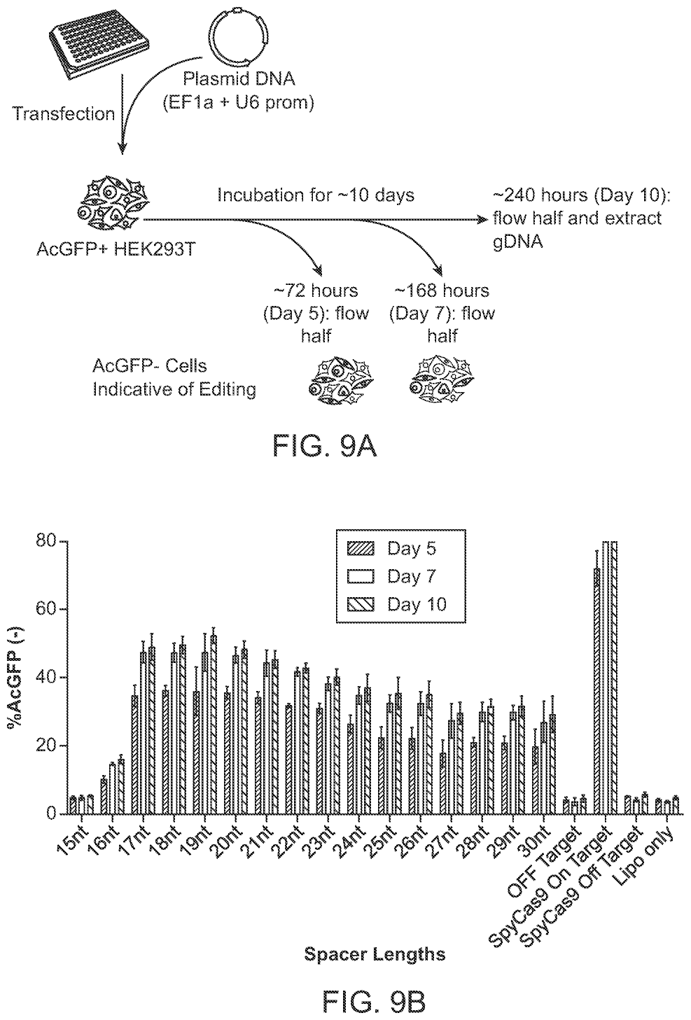

FIG. 9 illustrates CasΦ.12 cleavage in HEK293T cells and the effect of changing the spacer length on this cleavage. FIG. 9A provides a schematic of how CasΦ.12 cleavage activity was assessed in HEK293T cells. An Ac-GFP-expressing HEK293T cell line was transfected with a plasmid expressing CasΦ.12 and its crRNA targeting the Ac-GFP gene. CasΦ.12 cleavage was assessed by the reduction in Ac-GFP-expressing cells as assessed by flow cytometry. As shown in FIG. 9B, varying the spacer length varied the degree of CasΦ.12 cleavage. CasΦ.12 has a preference for a spacer length of 17 to 22 nucleotides in HEK293T cells, but longer spacers (up to 30 nucleotides was tested) also supported CasΦ.12 cleavage.

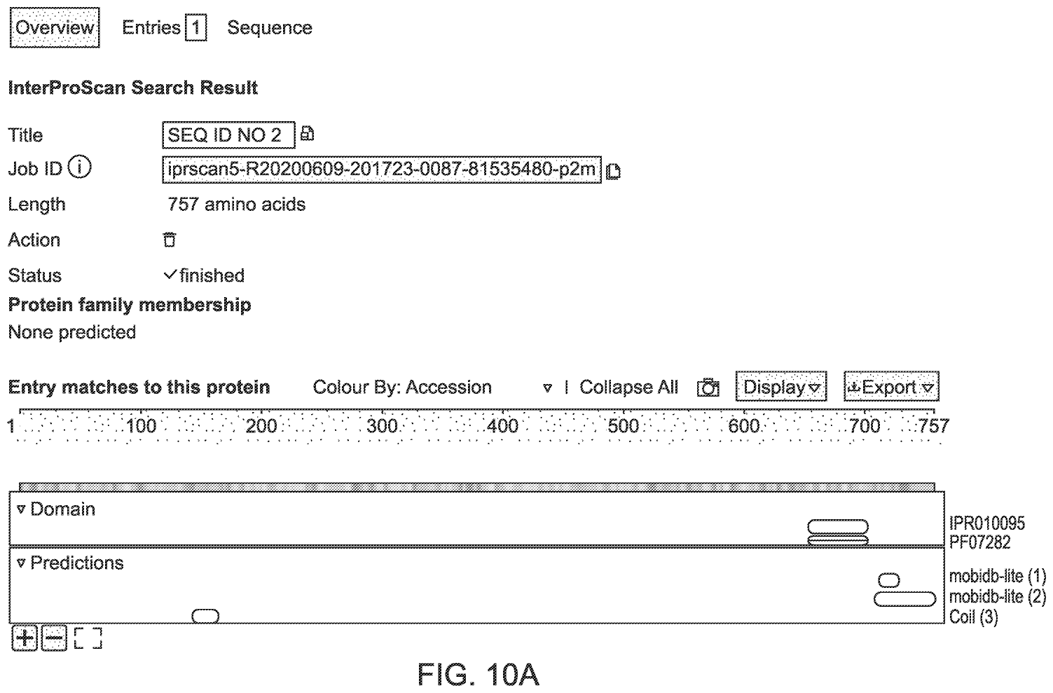

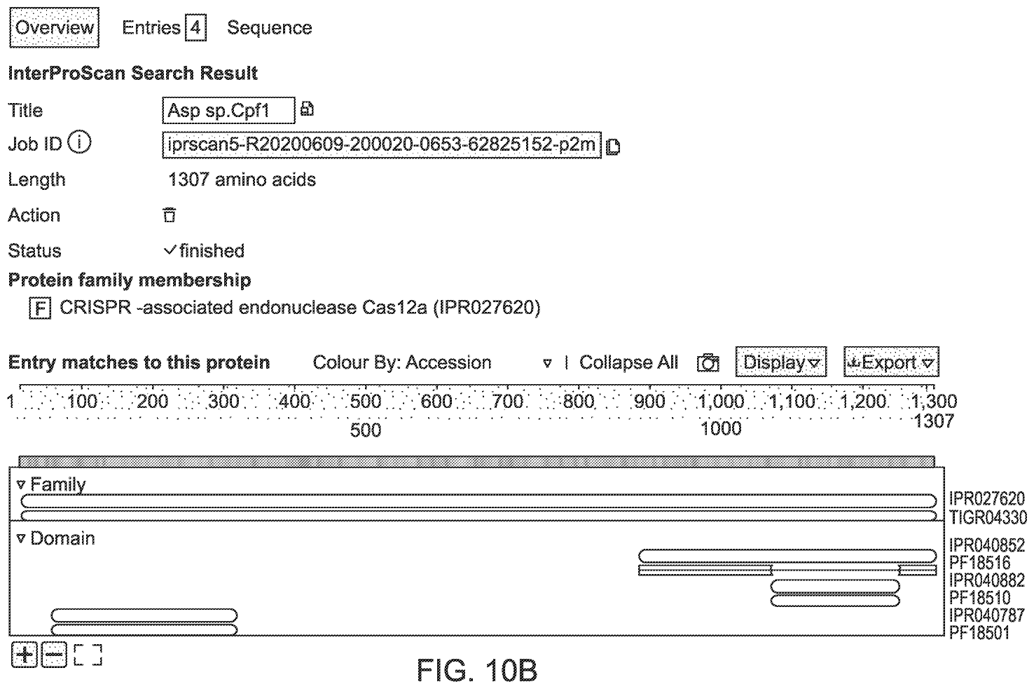

FIG. 10 illustrates that the CasΦ disclosed herein are a novel family of Cas nucleases. As shown in FIG. 10A, the InterPro database did not recognize CasΦ.2 as a protein family member. As a positive control, the InterPro database identified Acidaminococcus sp. (strain BV3L6) as a Cas12a protein family member, as shown in FIG. 10B.

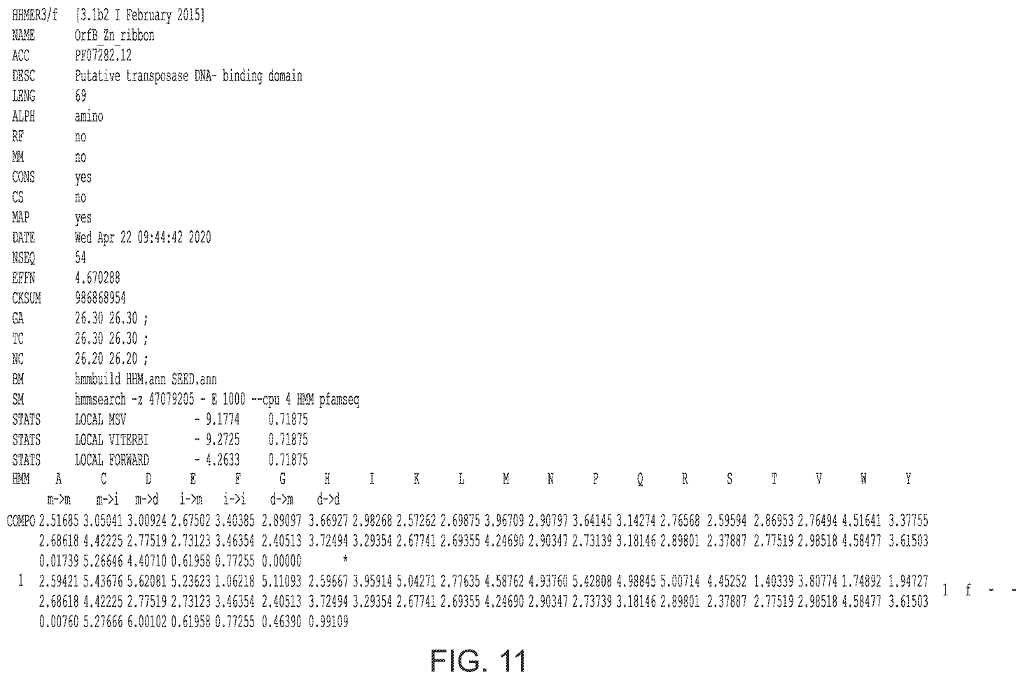

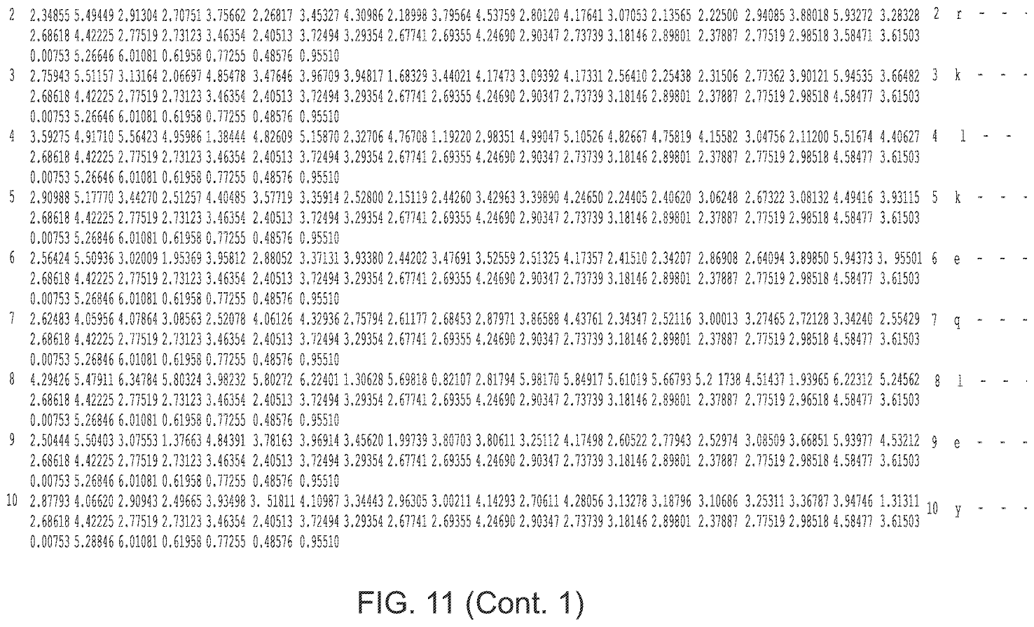

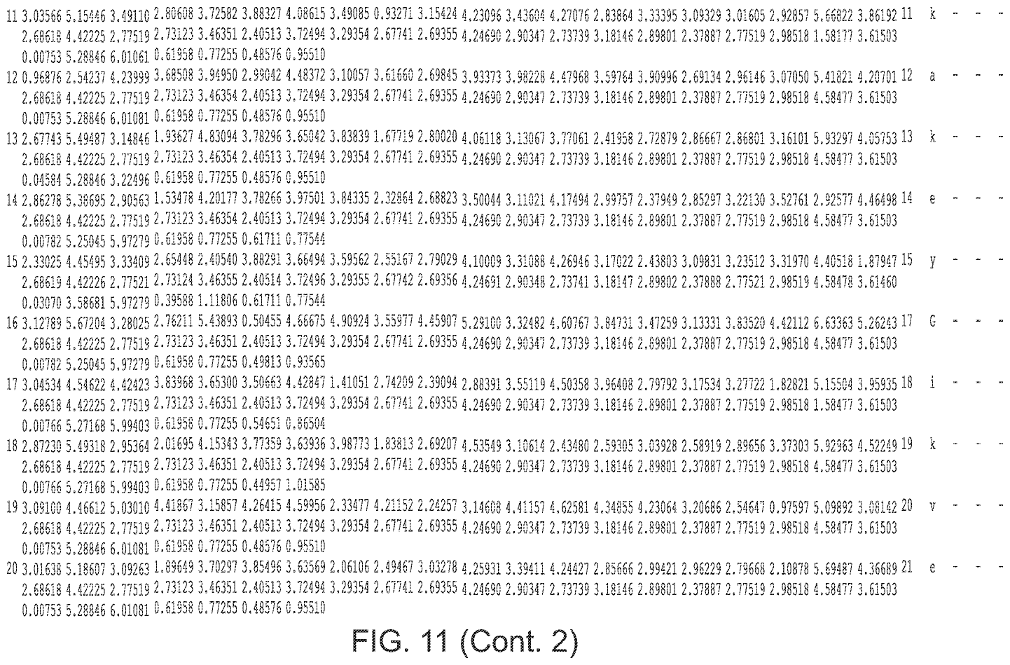

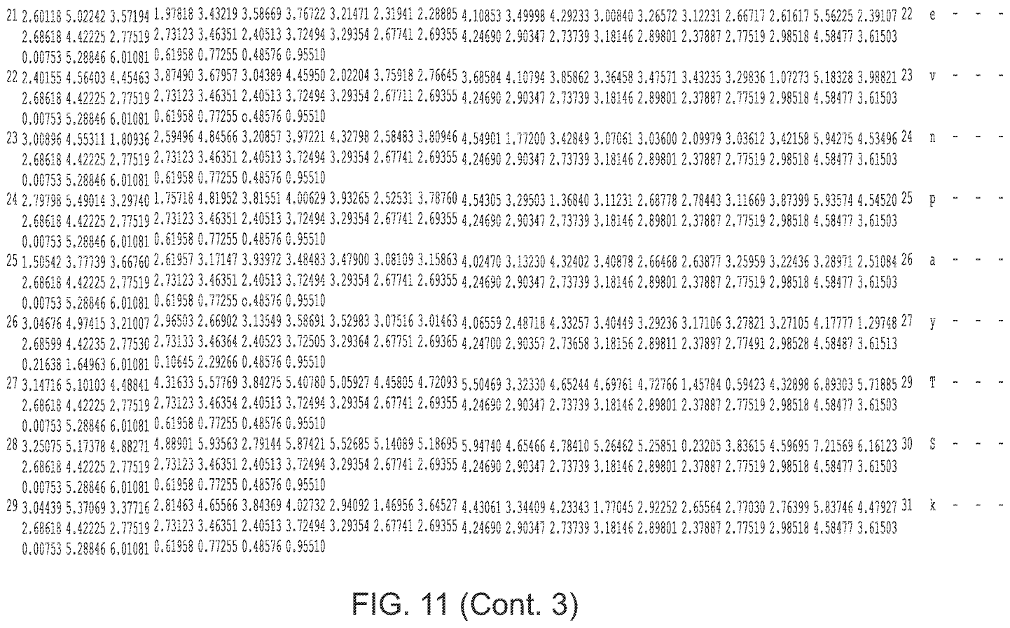

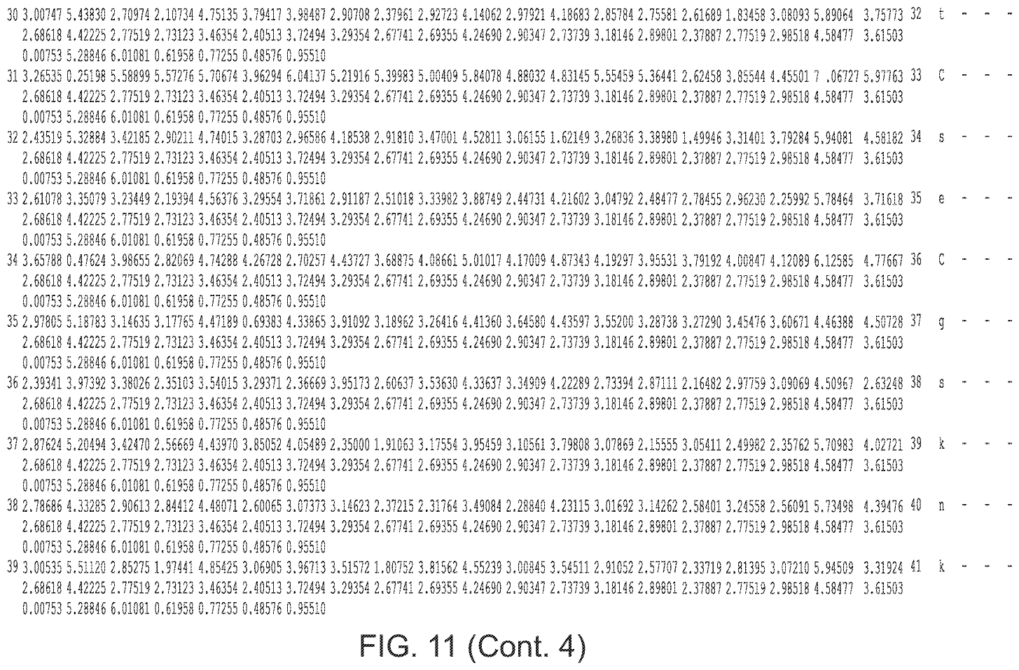

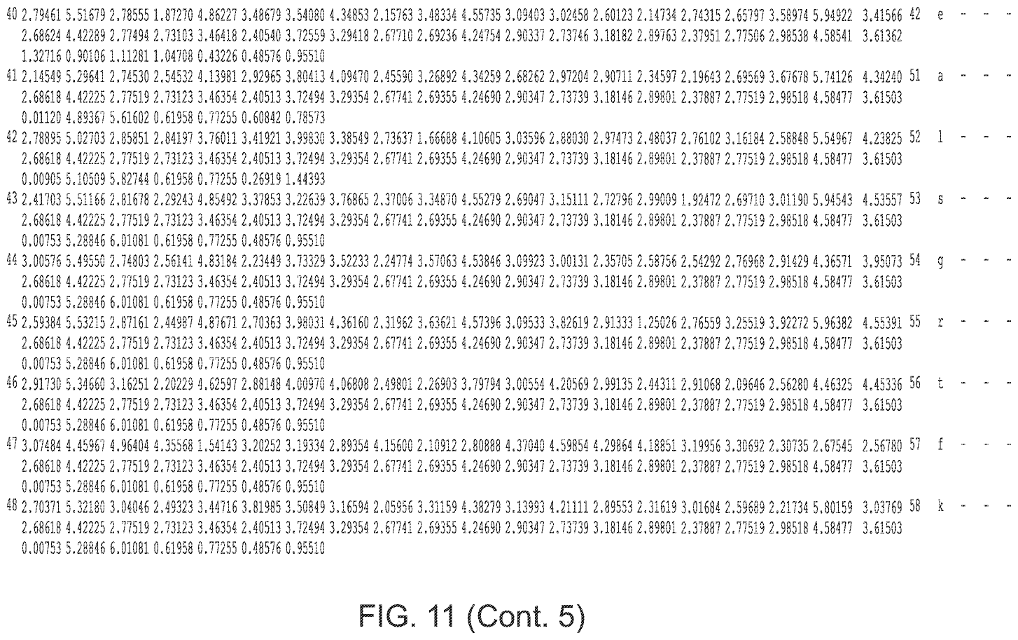

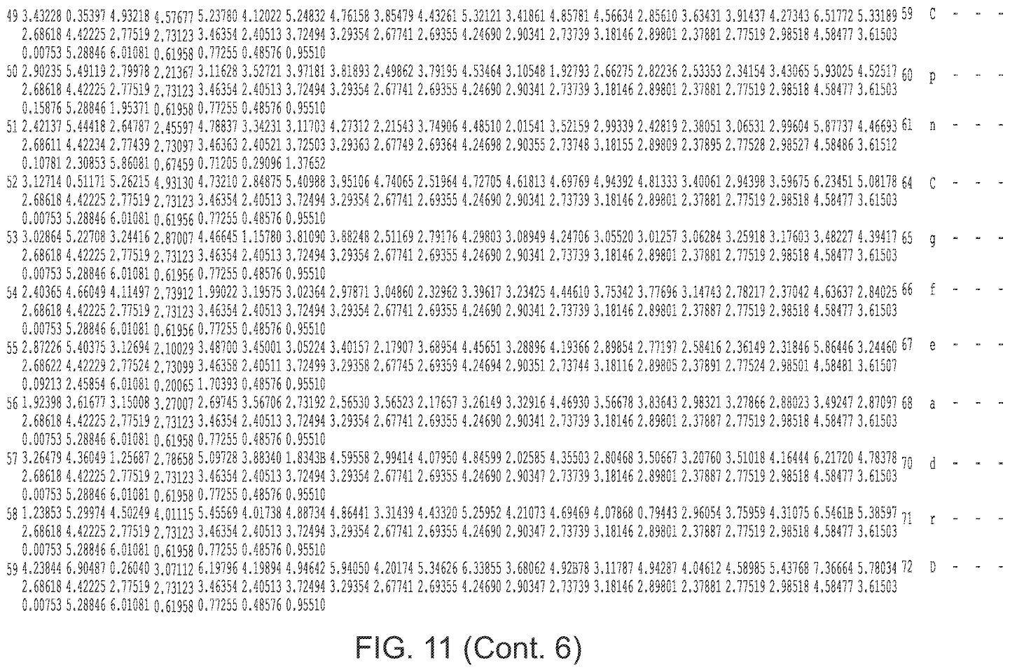

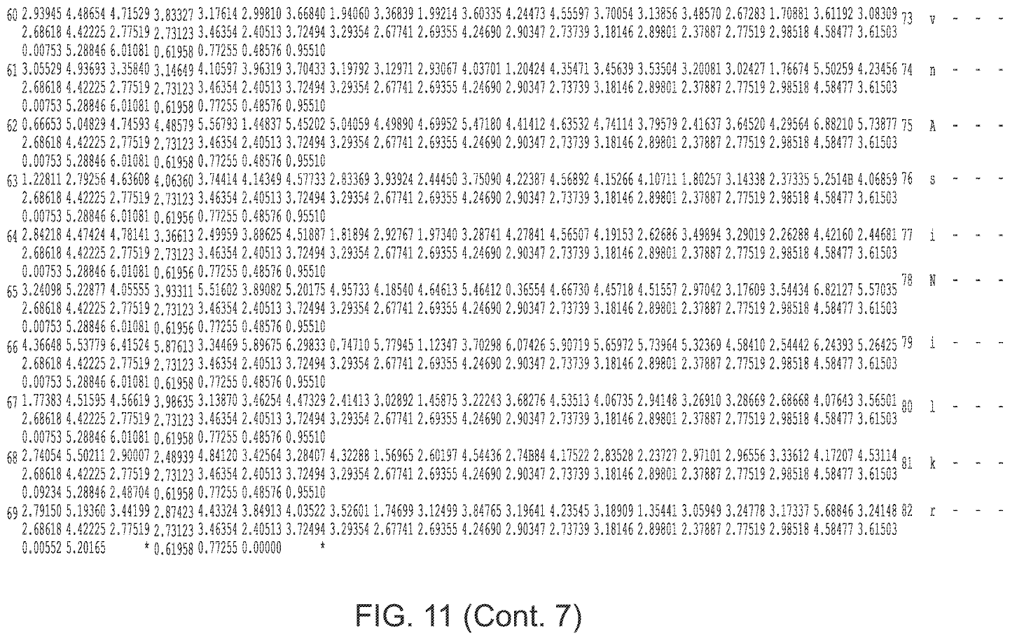

FIG. 11 illustrates the raw HMM for PF07282.

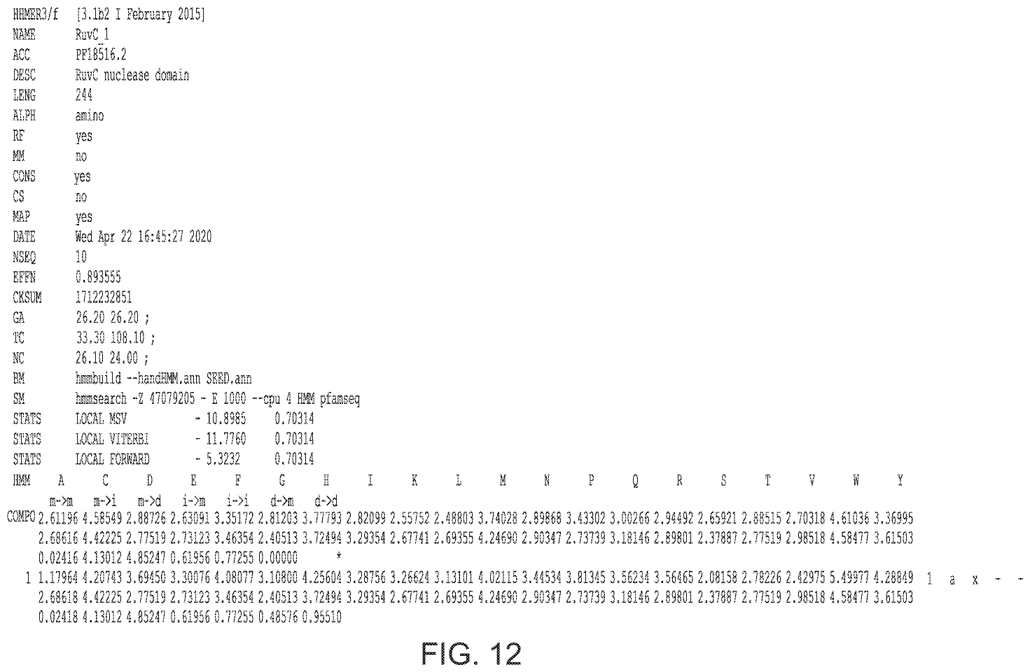

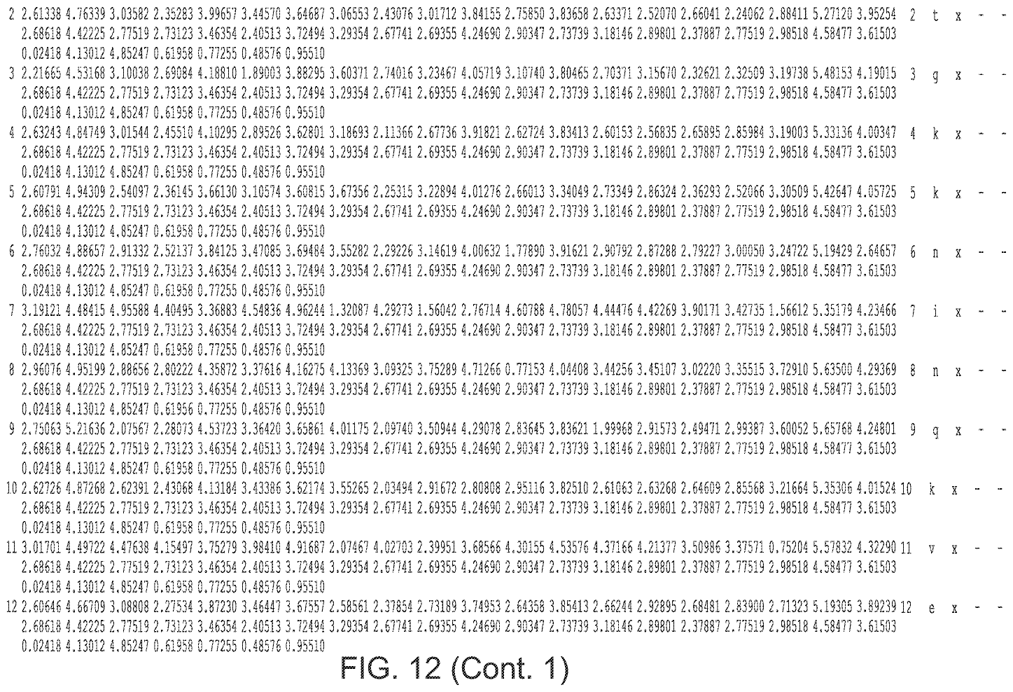

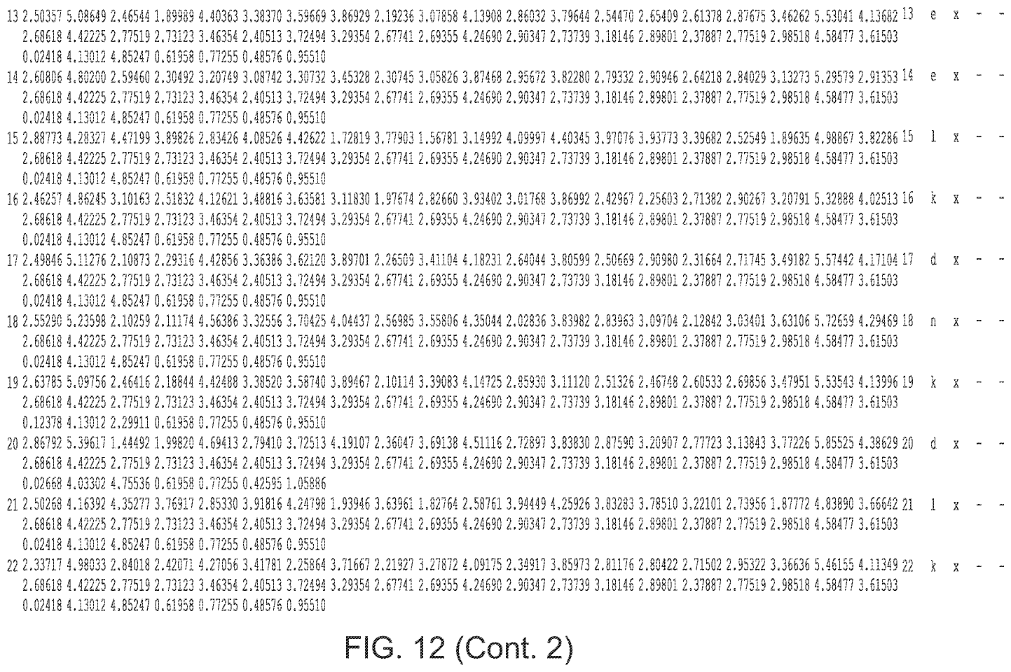

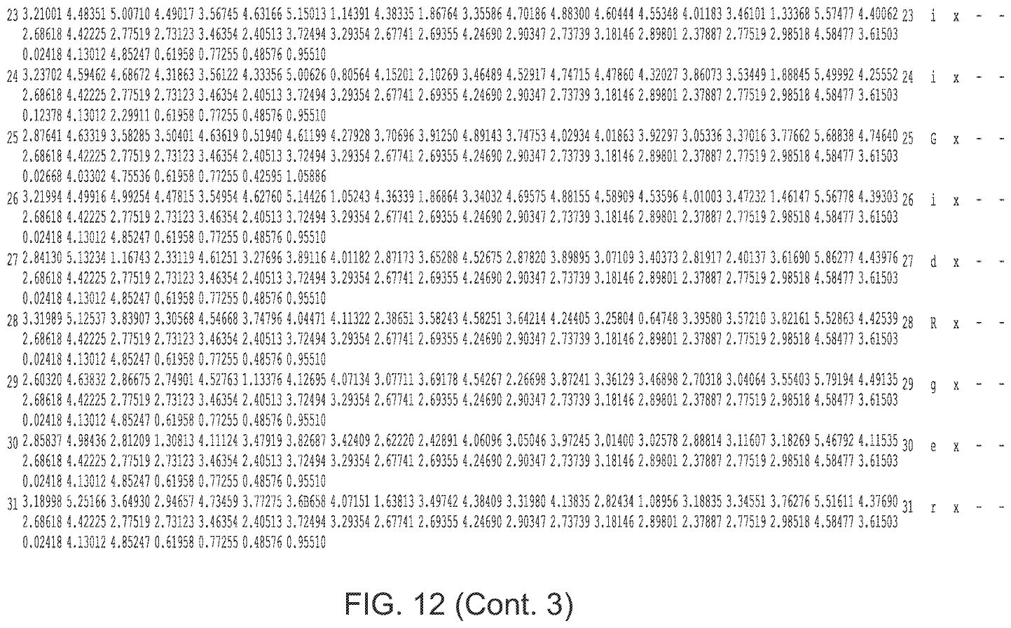

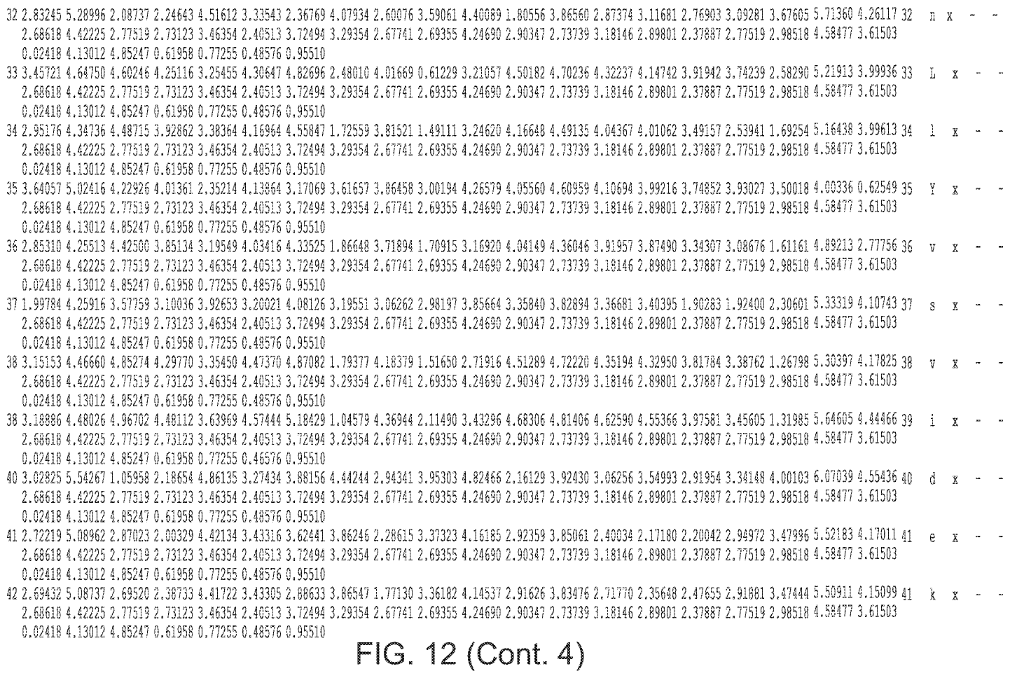

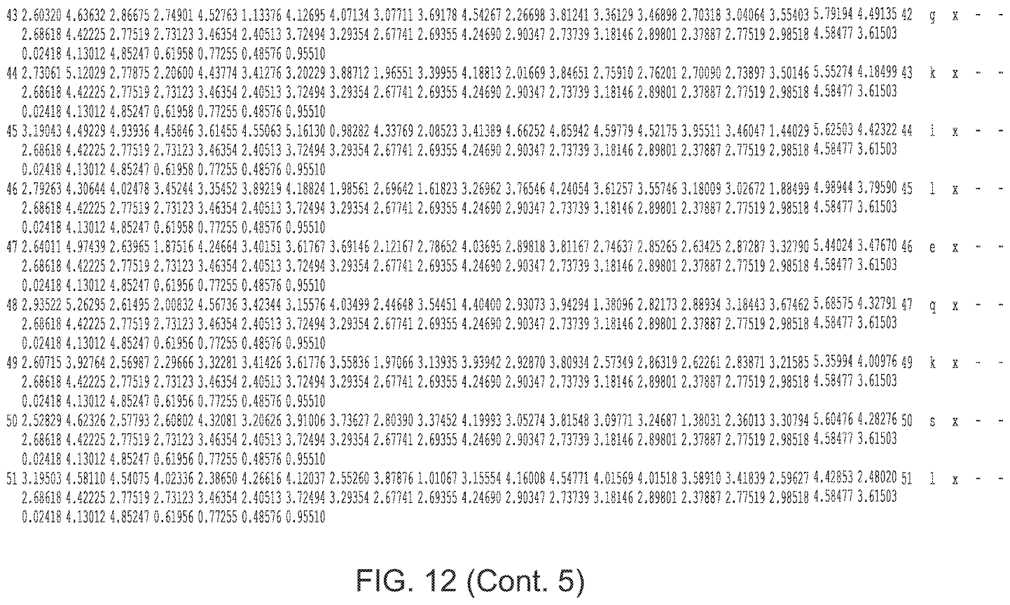

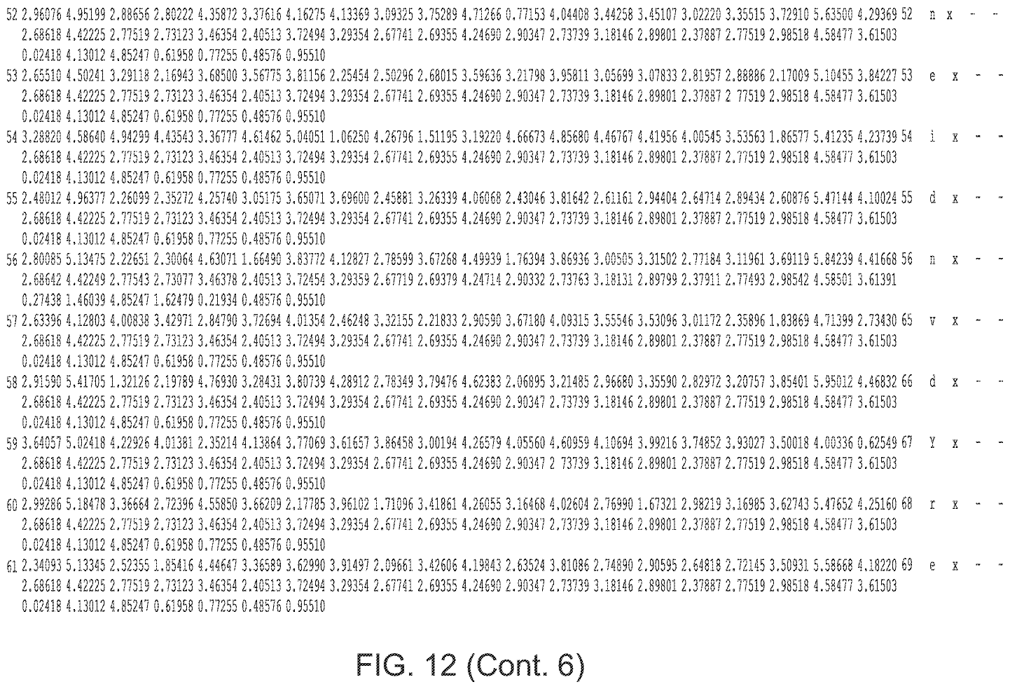

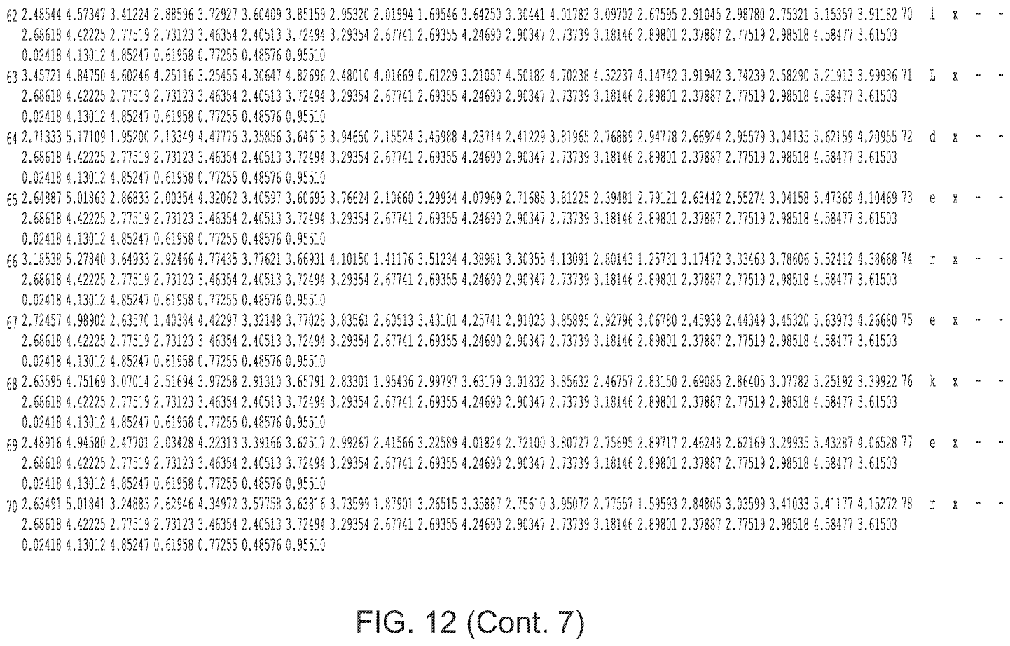

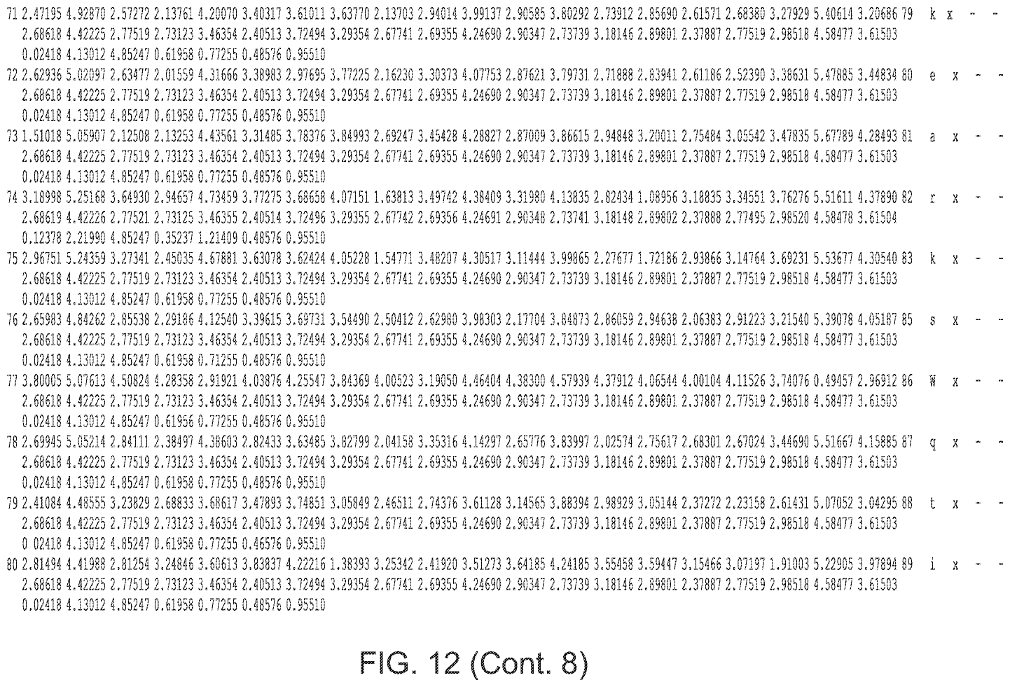

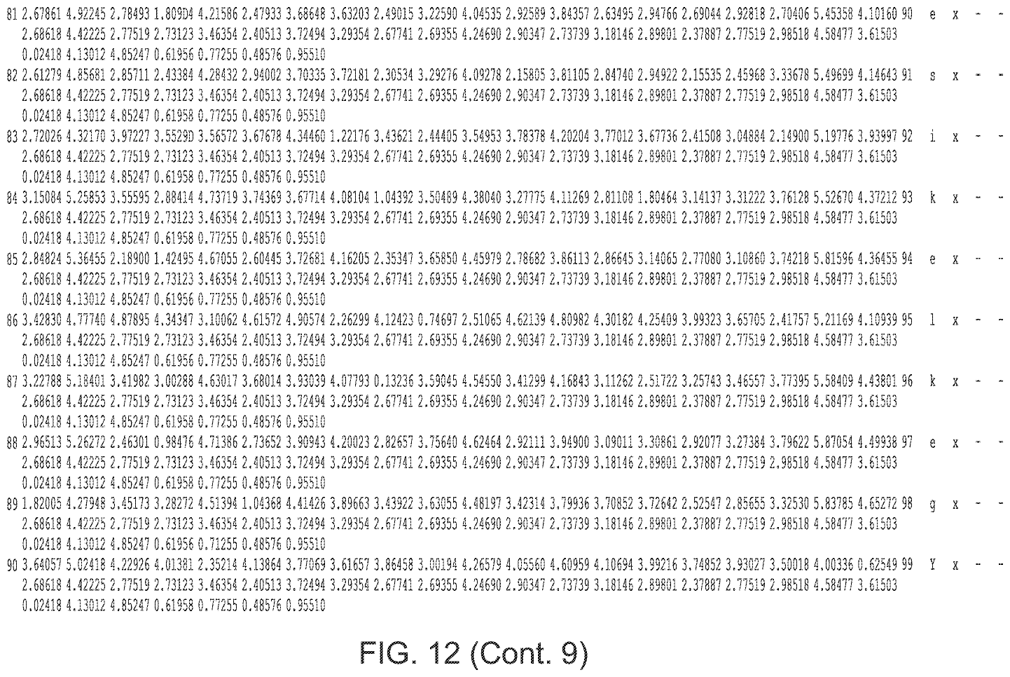

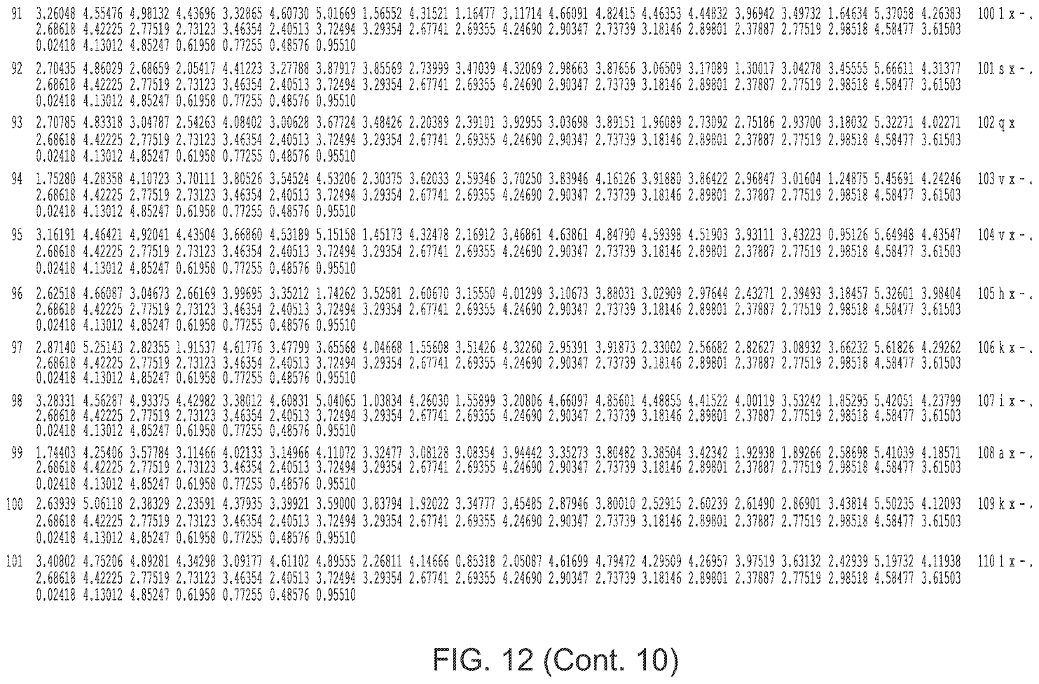

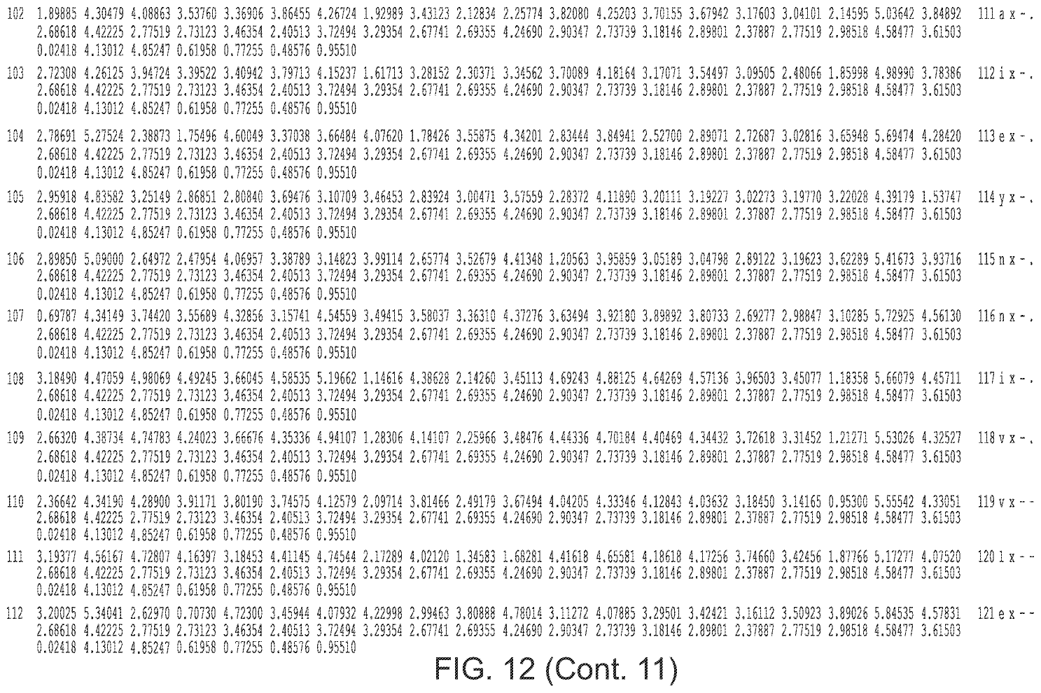

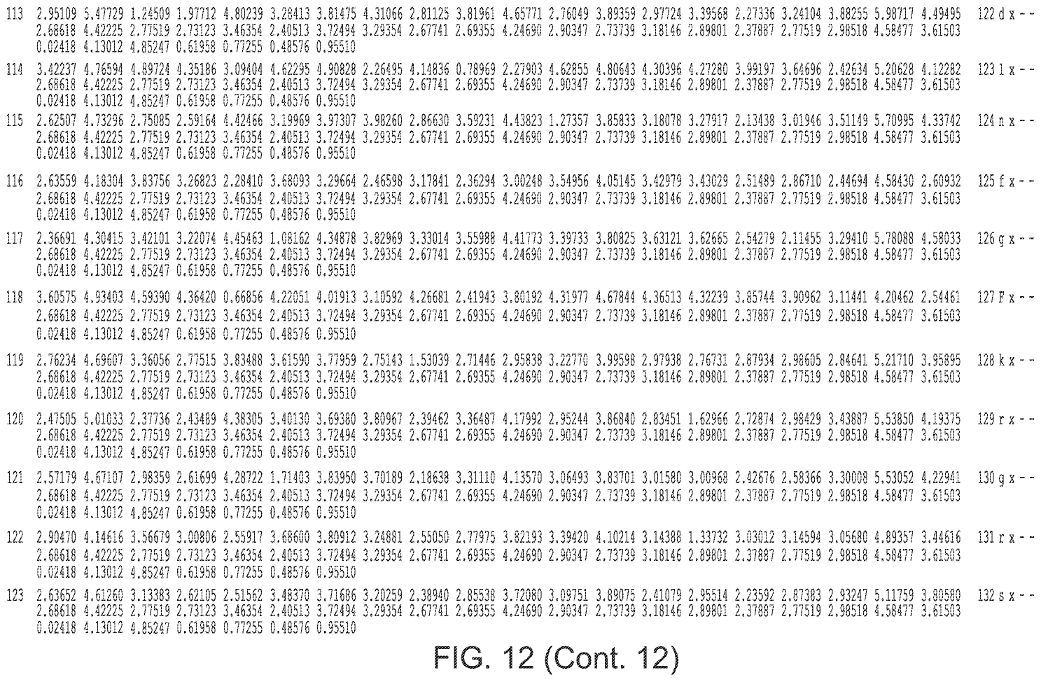

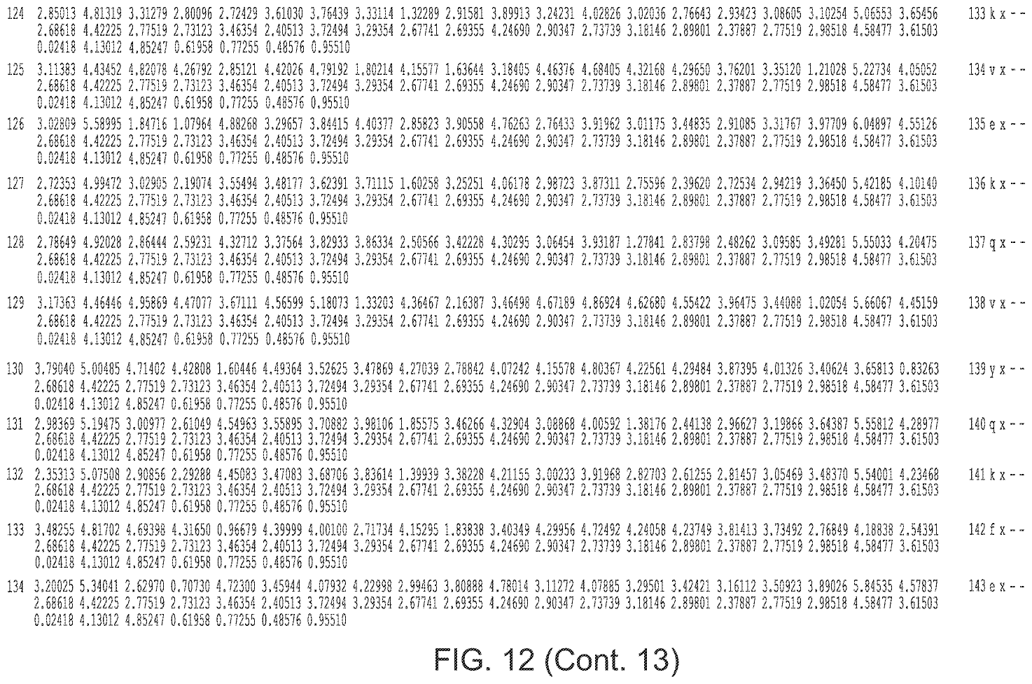

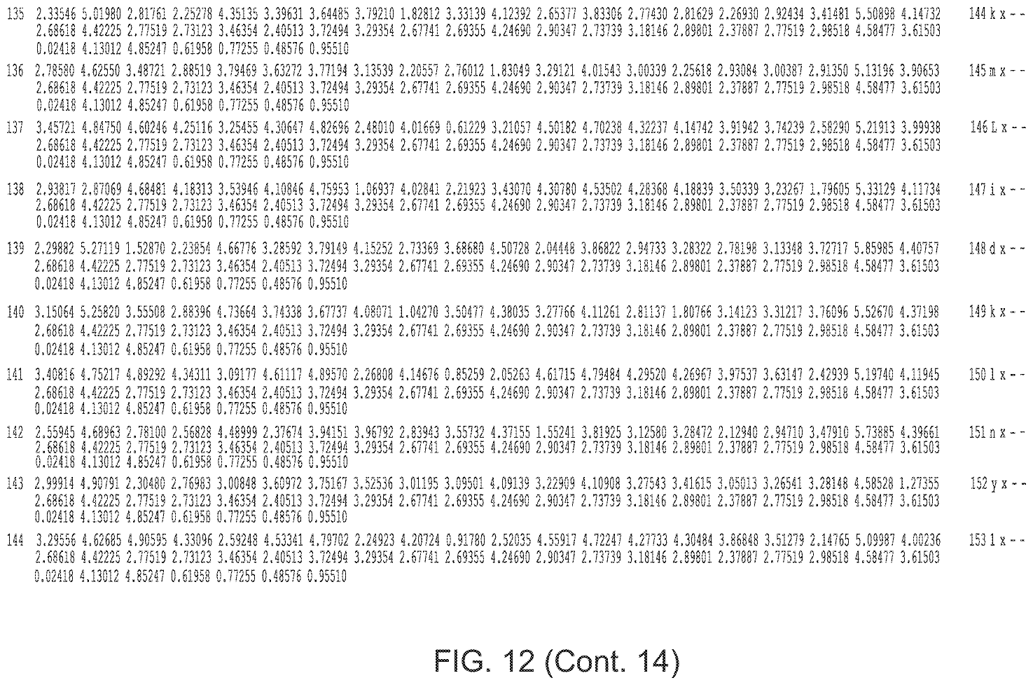

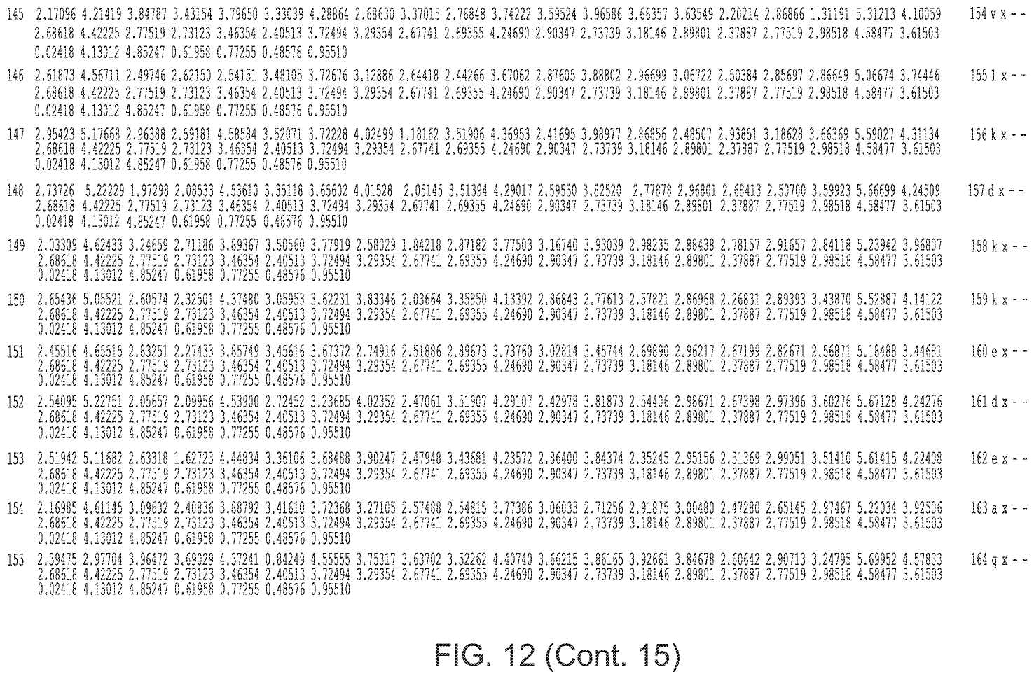

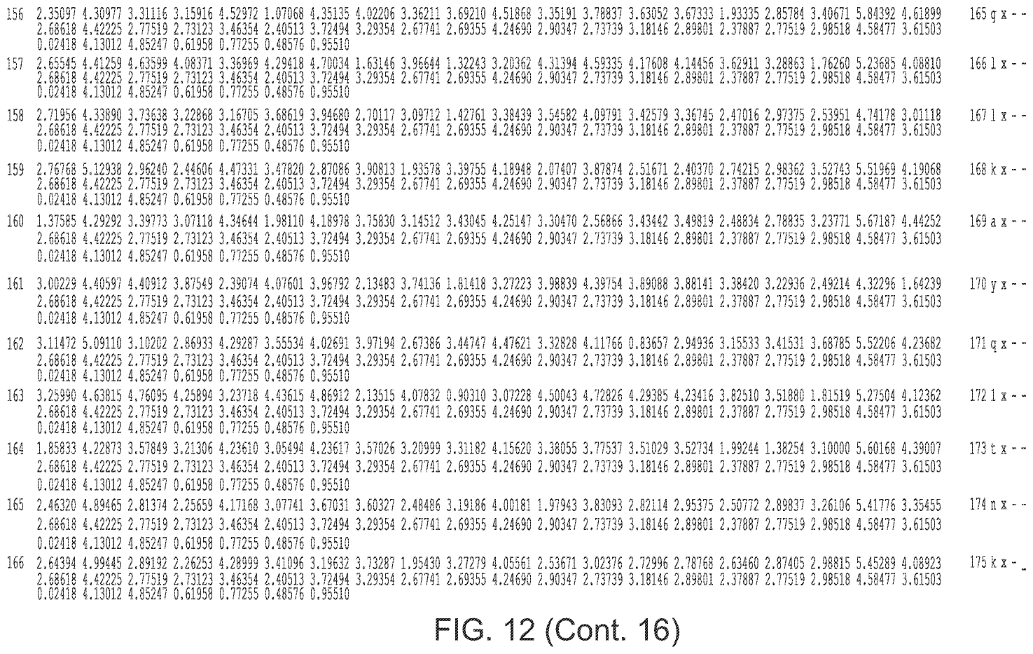

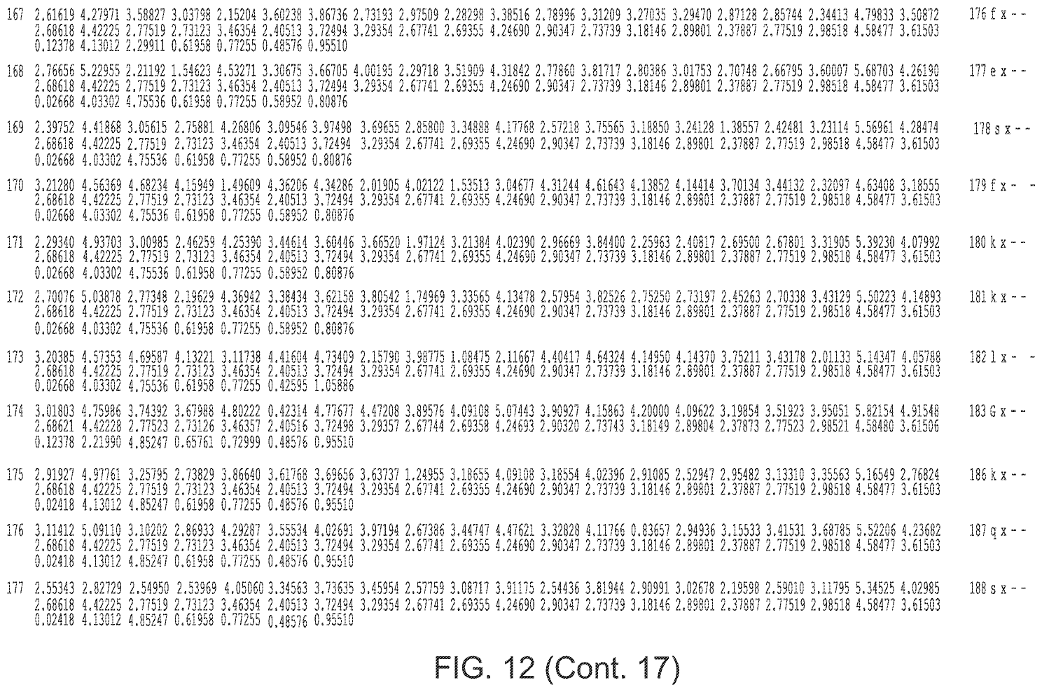

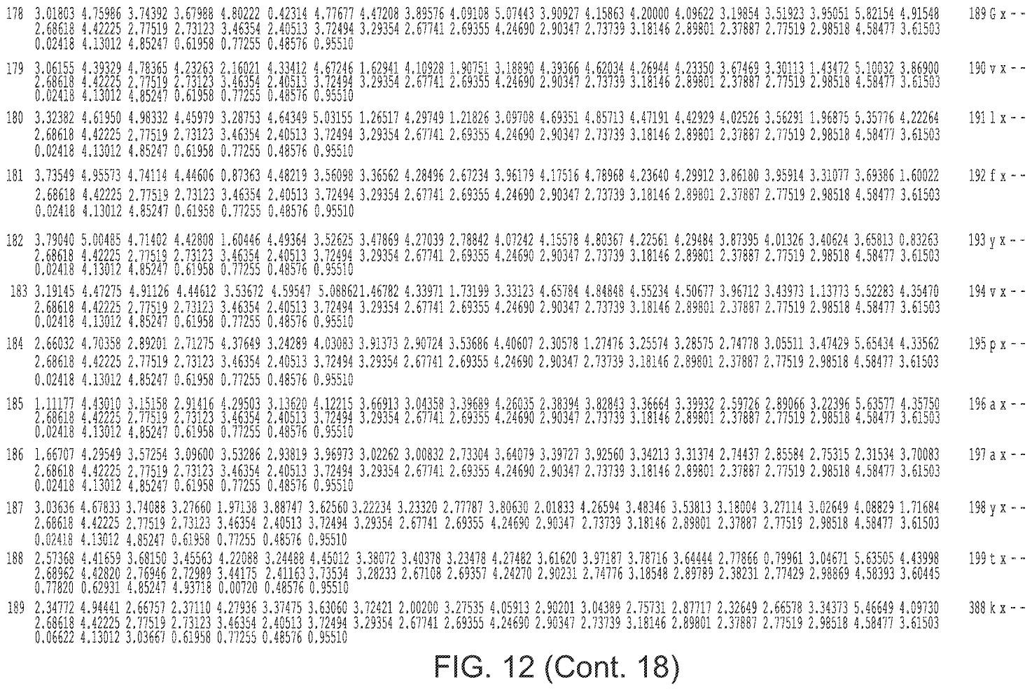

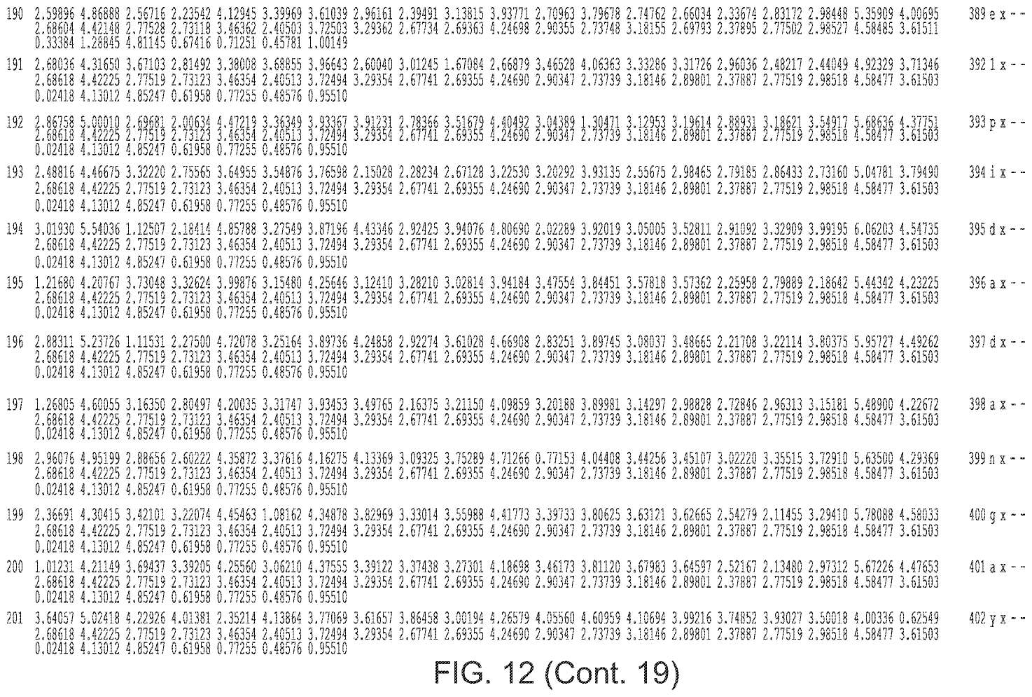

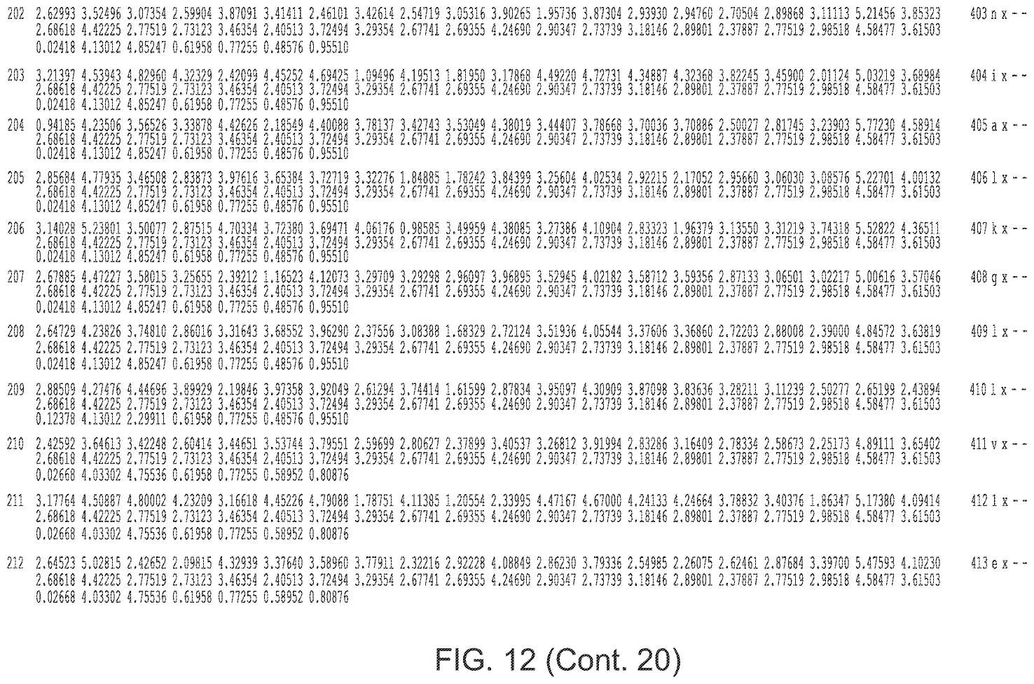

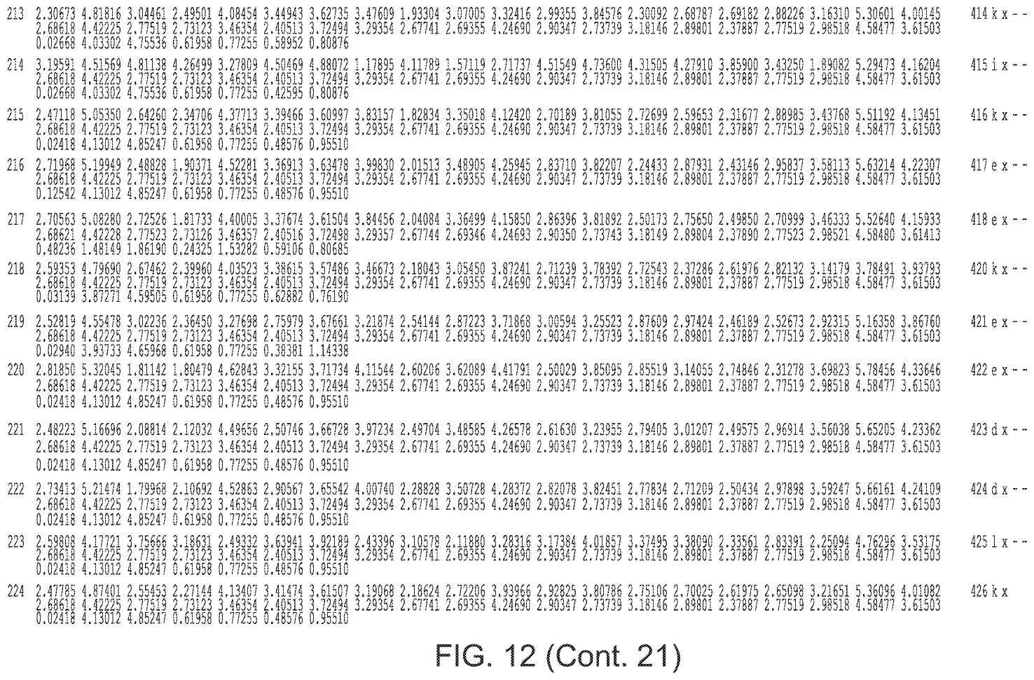

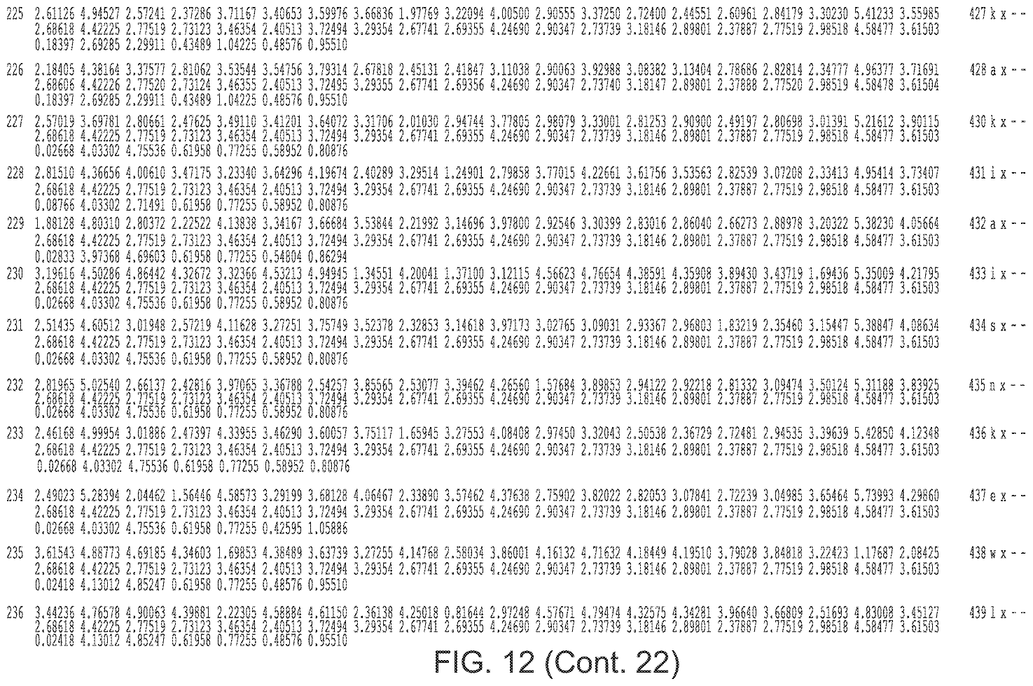

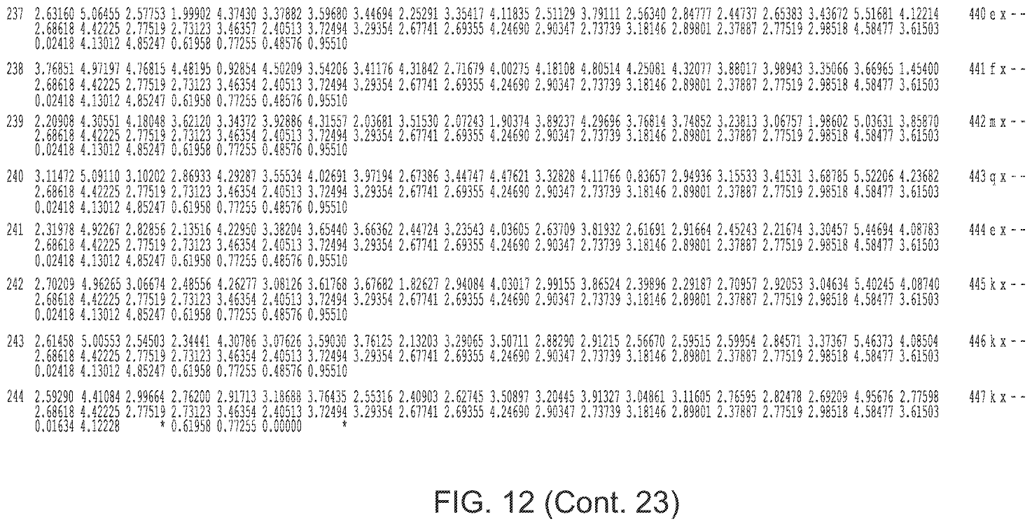

FIG. 12 illustrates the raw HMM for PF18516.

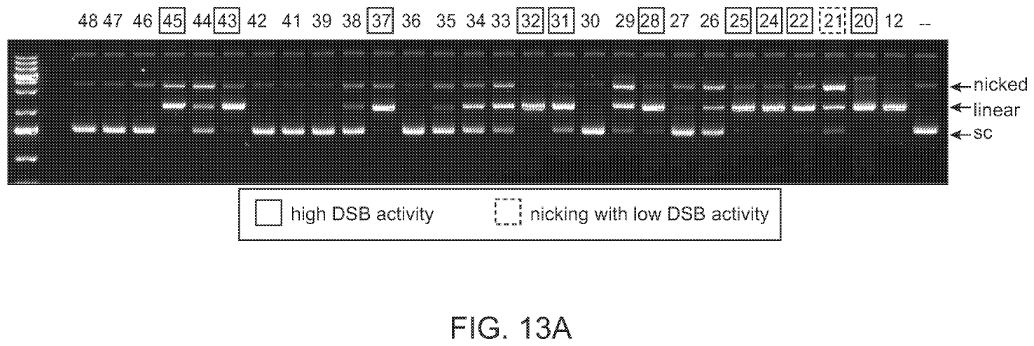

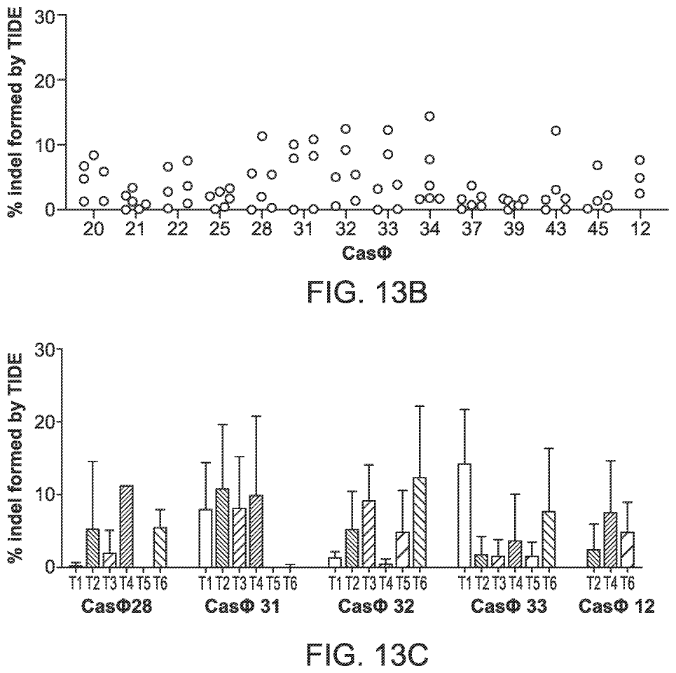

FIG. 13 illustrates the cleavage activity of CasΦ.19-CasΦ.48.

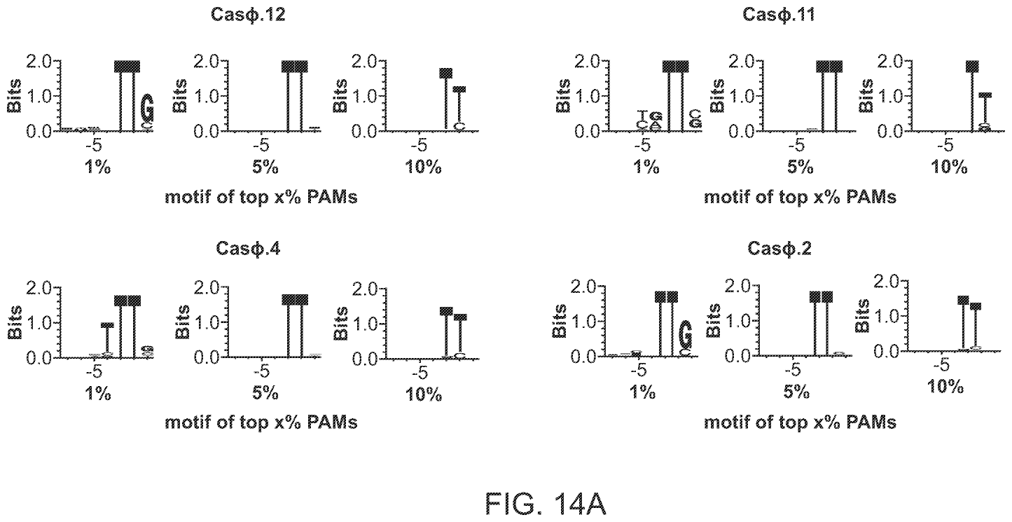

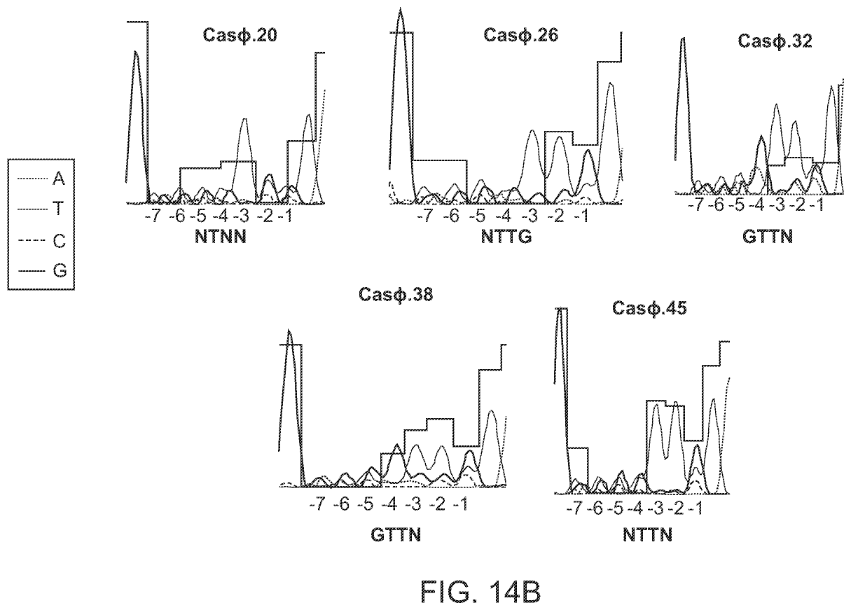

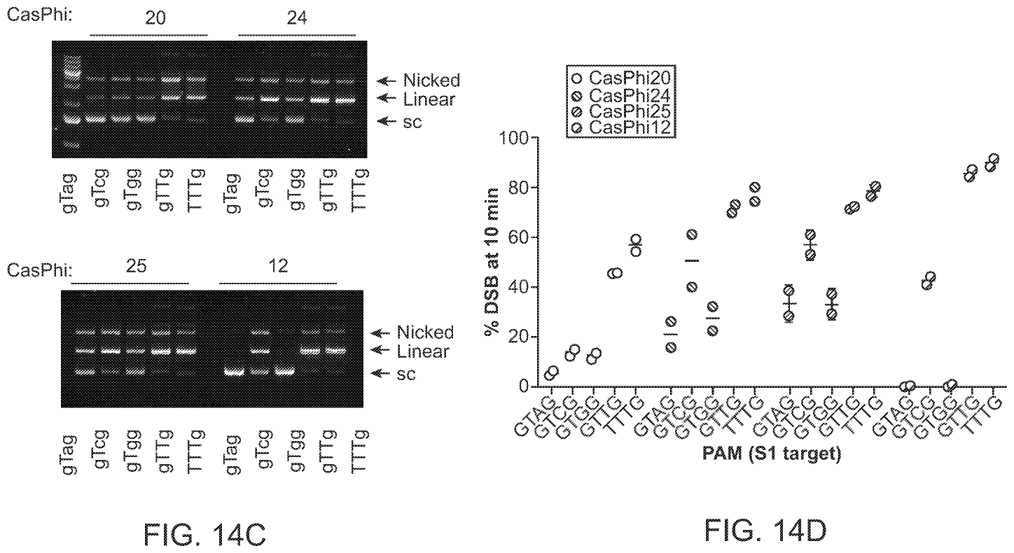

FIG. 14 illustrates the PAM requirement of CasΦ polypeptides. FIG. 14A shows the PAM requirement of CasΦ.2, CasΦ.4, CasΦ.11 and CasΦ.12. FIG. 14B shows the PAM requirement of CasΦ.20, CasΦ.26, CasΦ.32, CasΦ.38 and CasΦ.45. FIG. 14C shows the cleavage products from the assessment of the PAM requirement for CasΦ.20, CasΦ.24 and CasΦ.25. FIG. 14D shows the quantification of the raw data shown in FIG. 14C.

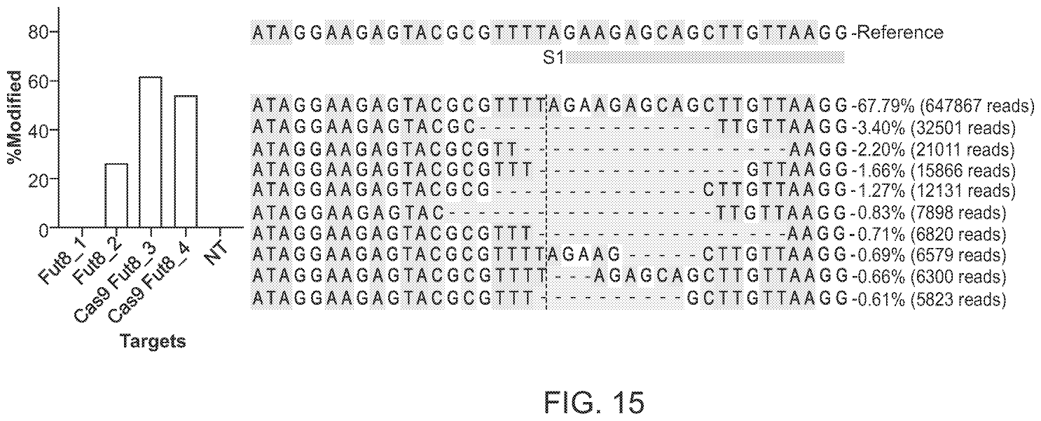

FIG. 15 illustrates endogenous gene editing in HEK293T cells.

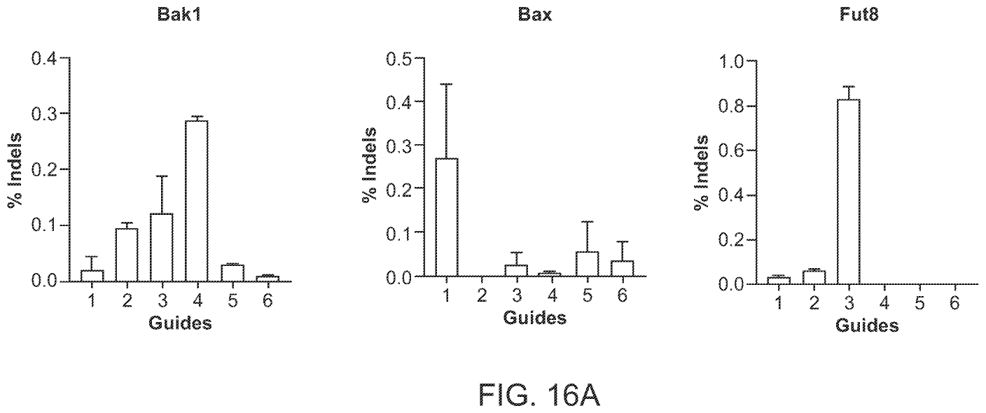

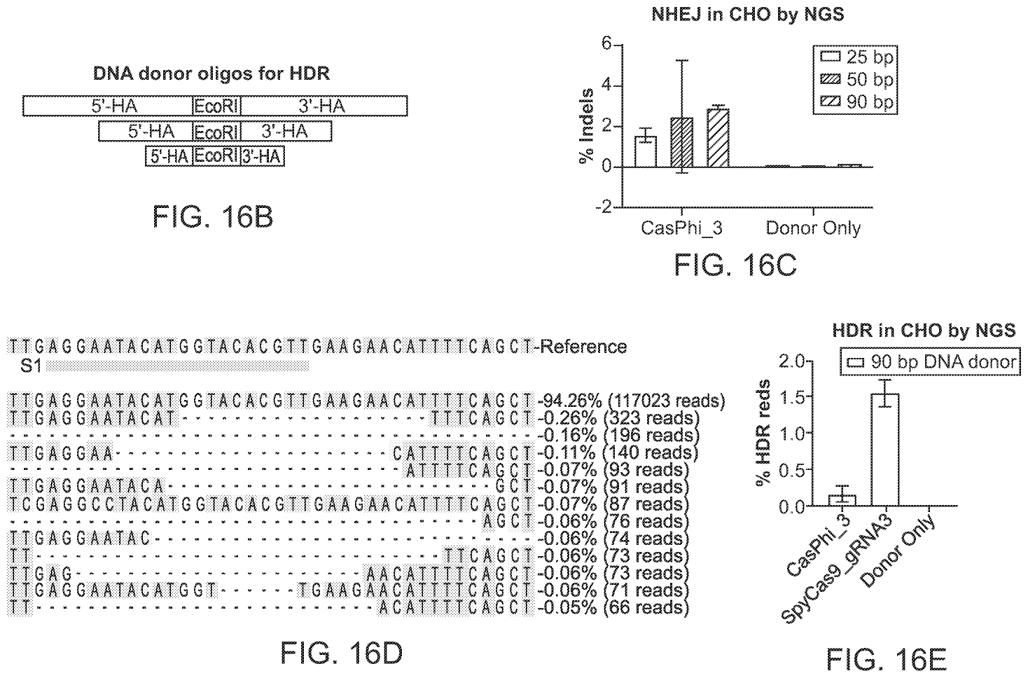

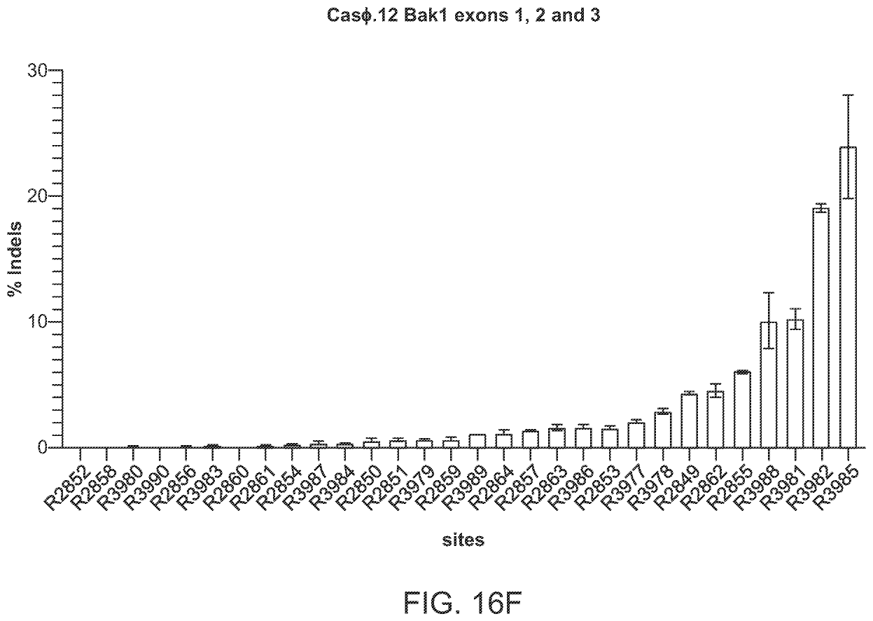

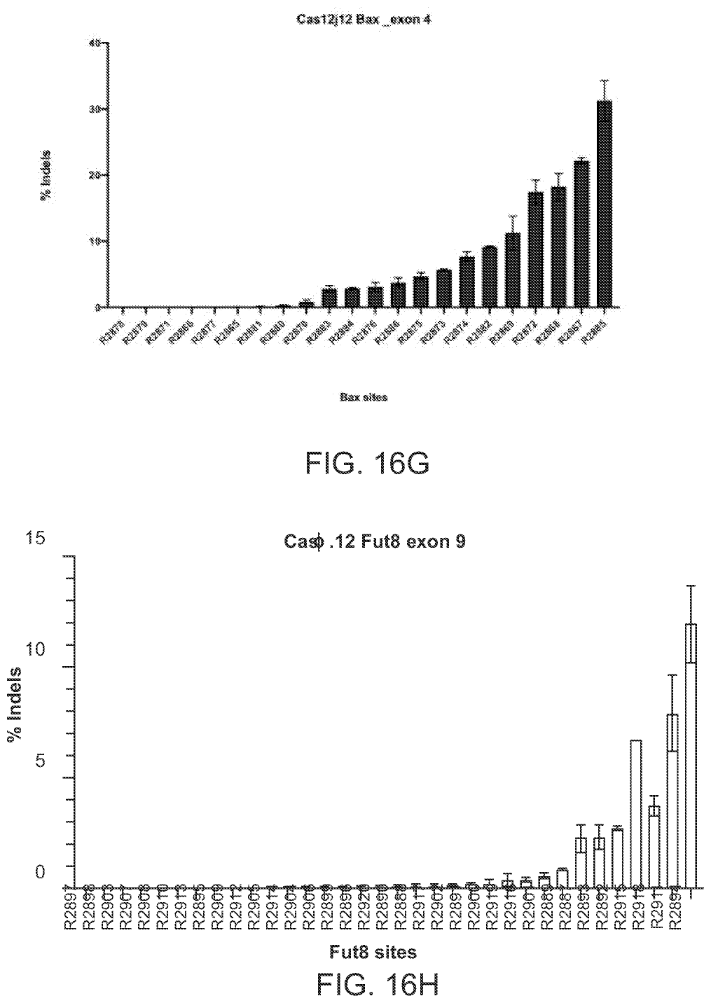

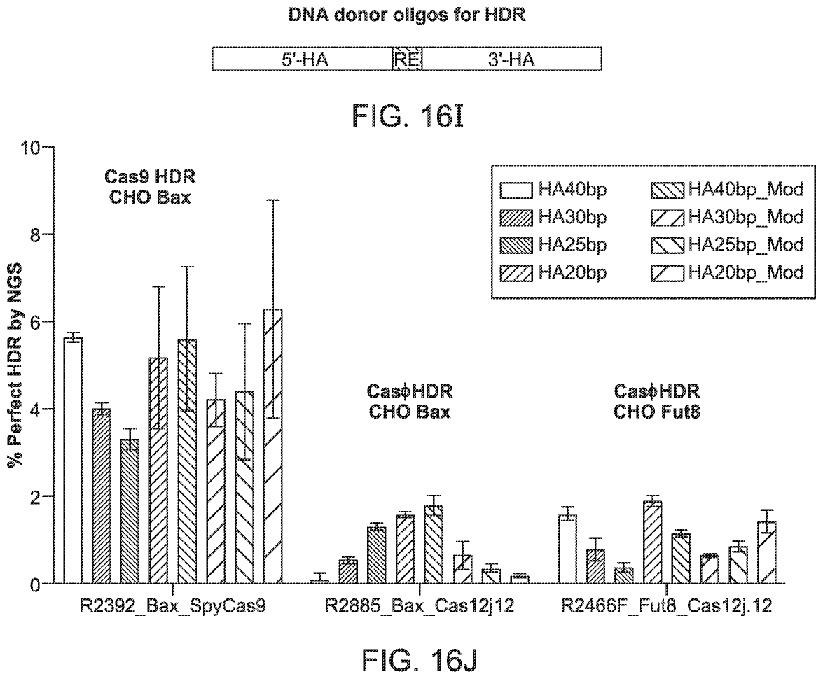

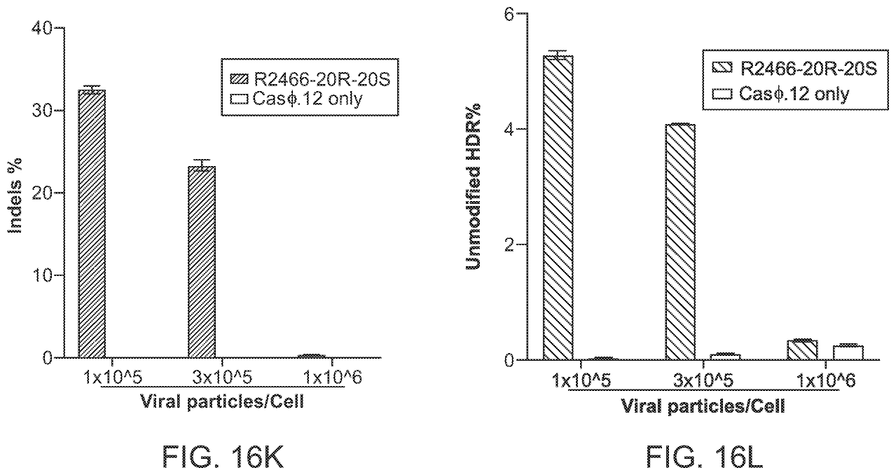

FIG. 16 illustrates endogenous gene editing in CHO cells. FIG. 16A shows CasΦ.12 mediated generation of insertion or deletion mutations (indel) in the endogenous Bak1, Bax and Fut8 genes. FIG. 16B shows the DNA donor oligos used to assess CasΦ.12 mediated gene editing via the homology directed repair pathway. FIG. 16C shows the detection of indels following delivery of CasΦ.12. FIG. 16D shows the sequence analysis for the data in FIG. 15C. FIG. 16E shows the detection of incorporated donor template following delivery of CasΦ.12 and a donor oligo. Further examples of CasΦ.12 mediated generation of indel mutations are shown in FIG. 16F, FIG. 16G and FIG. 1611 for Bak1, Bax and Fut8 genes, respectively. FIG. 161 shows the DNA donor oligos used to assess CasΦ.12 mediated gene editing via the homology directed repair pathway. FIG. 16J shows the frequency of HDR in CHO cells following delivery of either Cas9 and a gRNA targeting Bax, CasΦ.12 and a gRNA targeting Bax or CasΦ.12 and a gRNA targeting Fut8. FIG. 16K and FIG. 16L show the frequency of indel mutations and HDR, respectively, detected in CHO cells following delivery of CasΦ.12 and AAV6 DNA donors at the indicated number of viral genomes per cell (1×10\hat{ }5, 3×10\hat{ }5, or 1×10\hat{ }6).

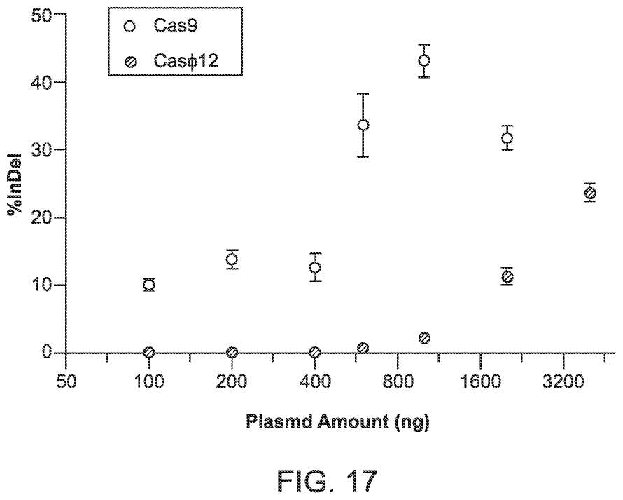

FIG. 17 illustrates endogenous gene editing in K562 cells.

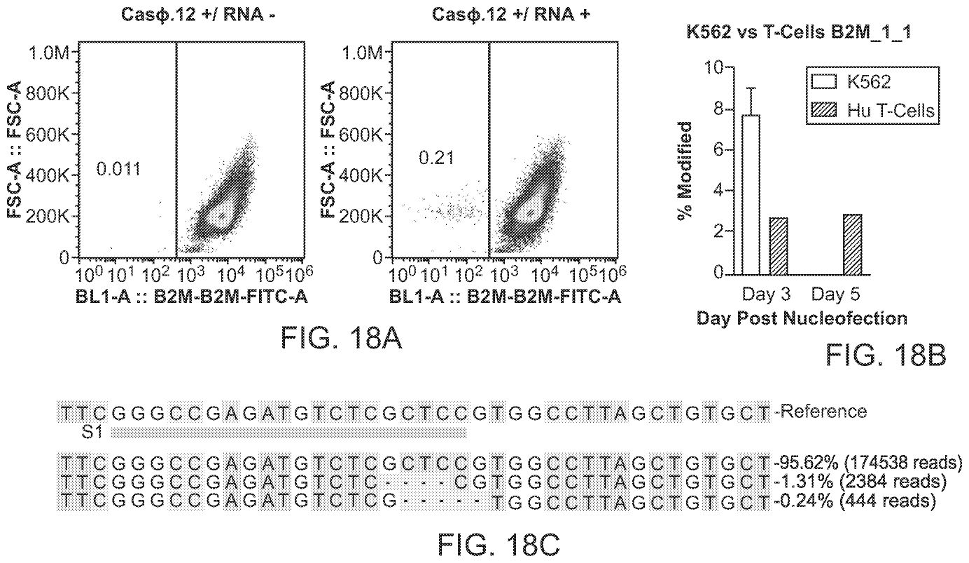

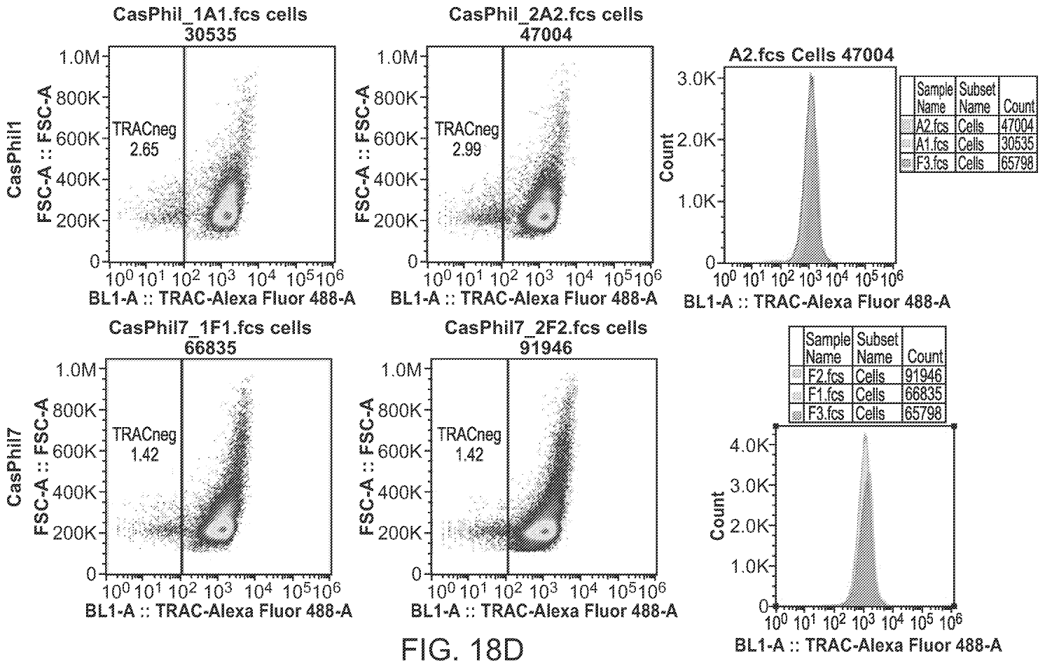

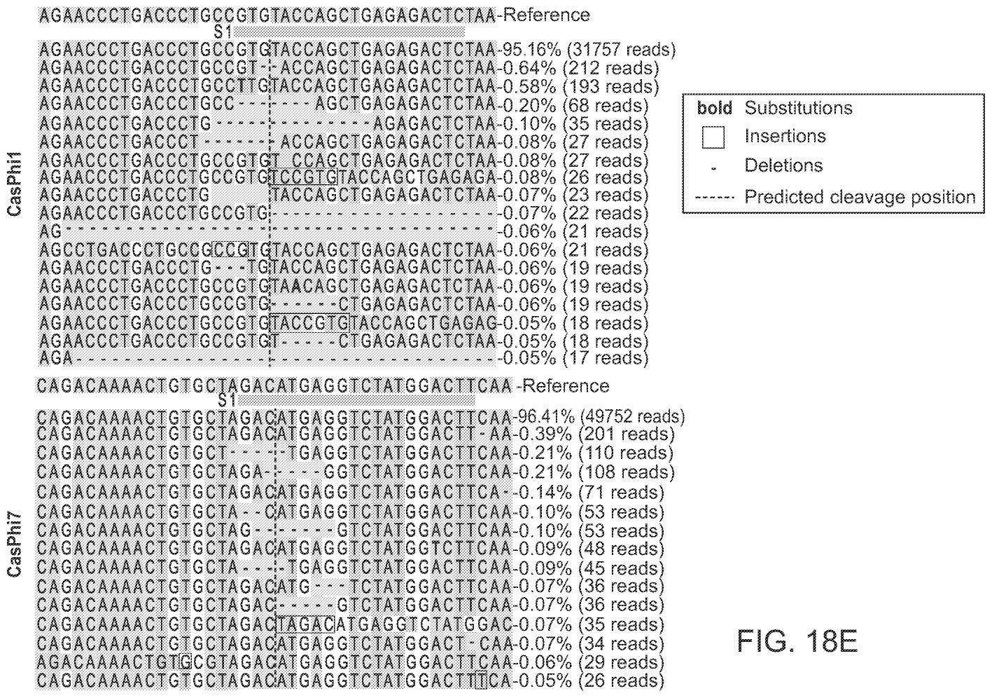

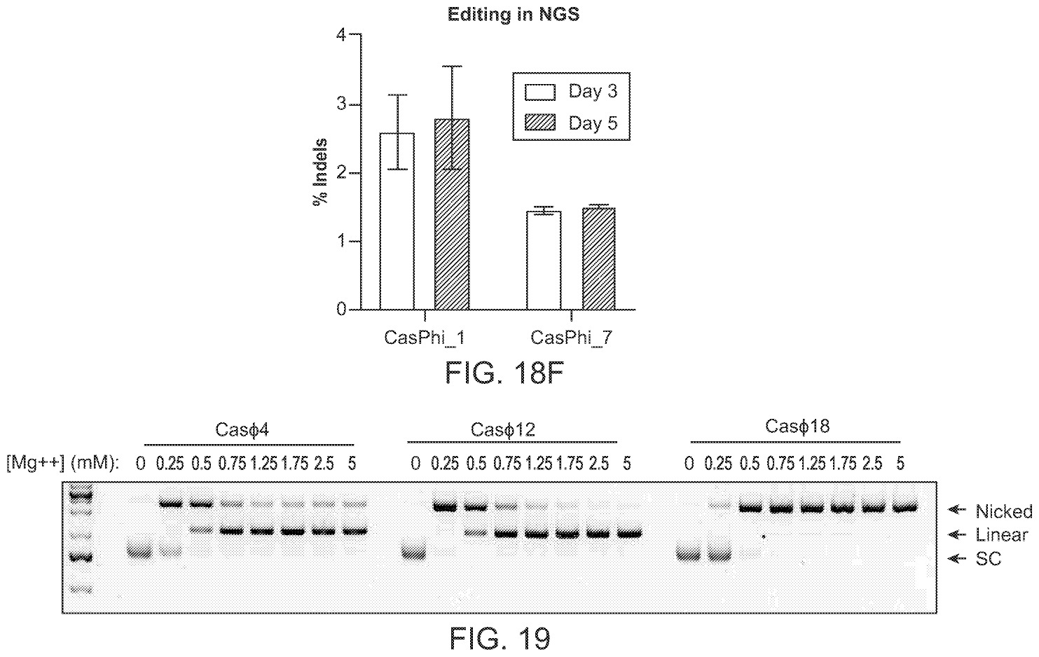

FIG. 18 illustrates endogenous gene editing in primary cells. FIG. 18A shows a flow cytometry analysis of T cells that have received CasΦ.12 with or without a gRNA targeting the beta-2 microglobulin gene. FIG. 18B shows the modification detected in K562 cells and T cells following delivery of CasΦ.12 and a gRNA targeting the beta-2 microglobulin gene. FIG. 18C shows the sequence analysis of the T cell population which received CasΦ.12 and the gRNA targeting the beta-2 microglobulin gene. FIG. 18D shows a flow cytometry analysis of T cells that have received CasΦ.12 with a gRNA targeting the T Cell Receptor Alpha Constant gene. FIG. 18E shows the sequence analysis of cell populations that received CasΦ.12 with a gRNA targeting the T Cell Receptor Alpha Constant gene. FIG. 18F shows the quantification of indels detected by sequence analysis.

FIG. 19 illustrates the cleavage of the second DNA strand by CasΦ nucleases in a separable reaction step to the cleavage of the first DNA strand.

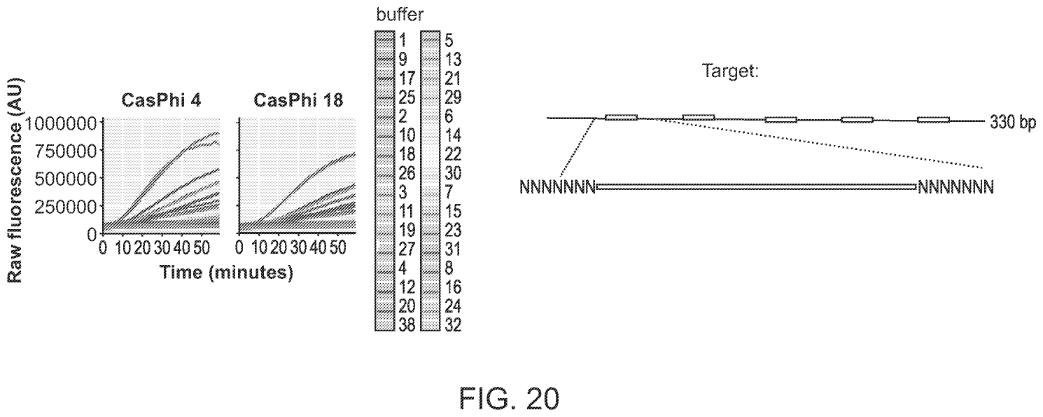

FIG. 20 illustrates the trans cleavage of ssDNA by CasΦ nucleases in a detection assay.

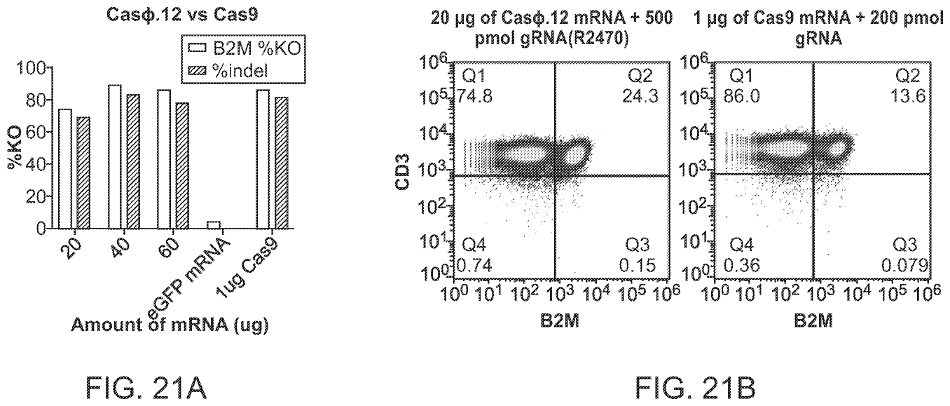

FIG. 21 illustrates the CasΦ.12-mediated efficiency is comparable to that of Cas9. FIG. 21A shows the frequency of indel mutations and quantification of B2M knockout cells from flow cytometry panels in FIG. 21B.

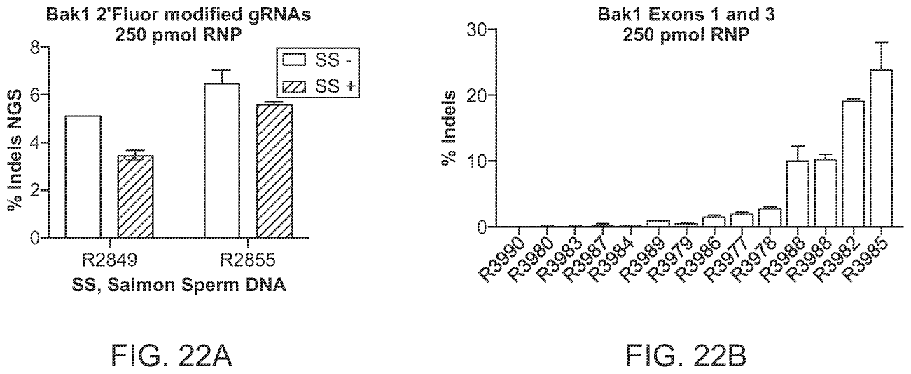

FIG. 22 illustrates the identification of optimized gRNAs for genome editing with CasΦ.12 in CHO cells. FIG. 22A shows the frequency of indel mutations induced by CasΦ.12 polypeptides complexed with a 2′fluoro modified gRNA. FIG. 22B shows further CasΦ.12 RNP complexes that can mediate genome editing in CHO cells.

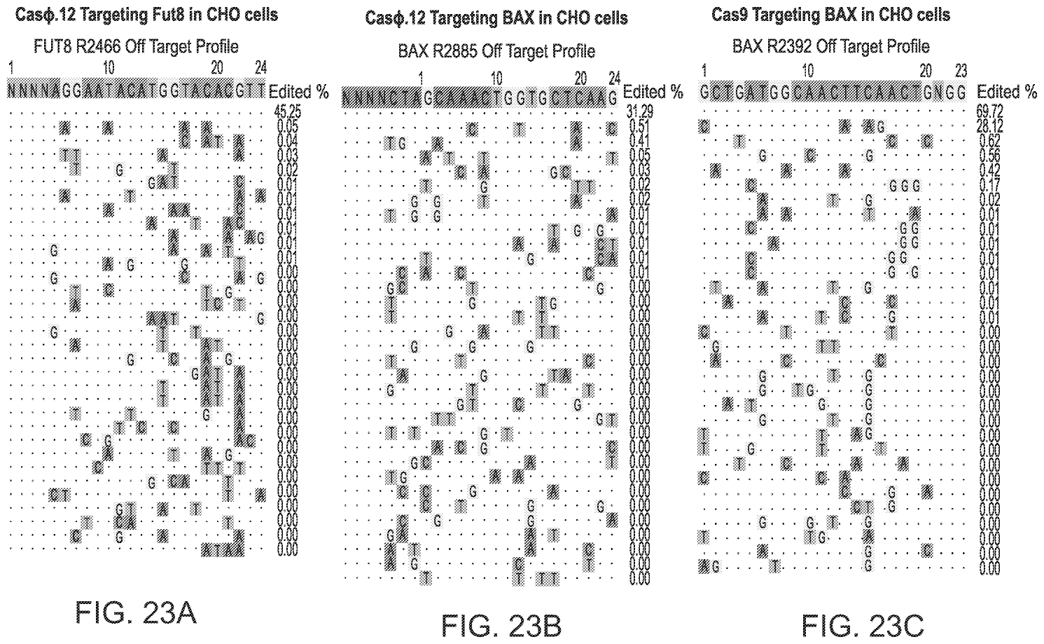

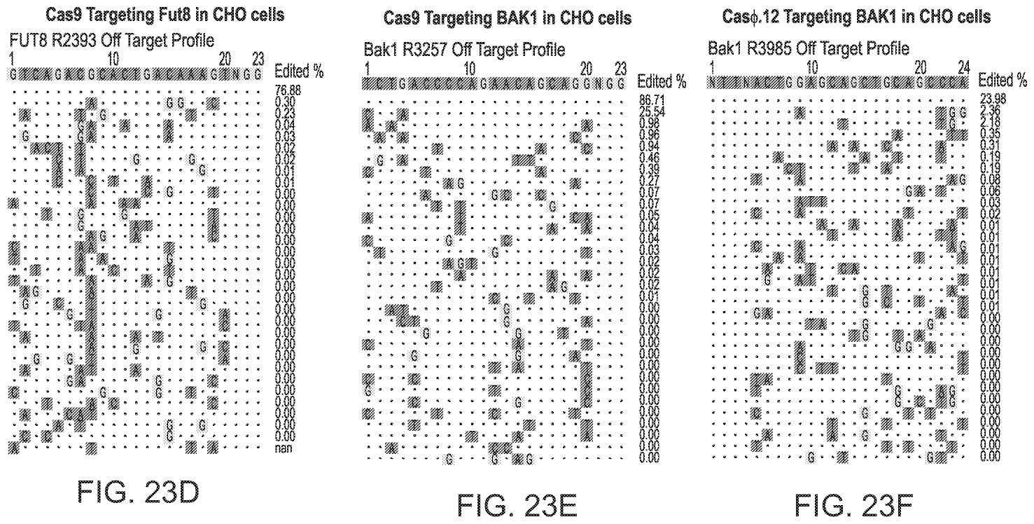

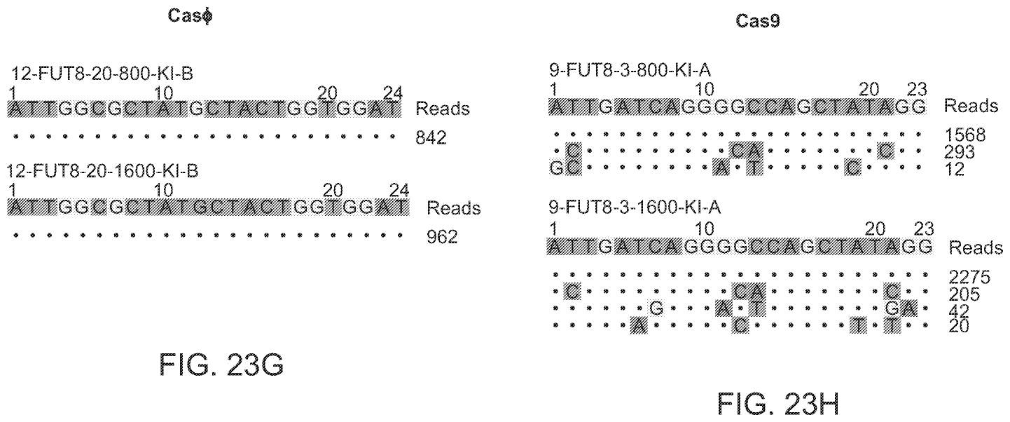

FIG. 23 illustrates minimal off-target CasΦ.12-mediated genome editing in CHO and HEK293 cells. FIG. 23A-F are off-target analysis InDel validation from a list of potential off-target sites based on in-silico computational predictions. FIG. 23A shows CasΦ.12 targeting Fut8, FIG. 23B shows CasΦ.12 targeting BAX, FIG. 23C shows Cas9 targeting BAX, FIG. 23D shows Cas9 targeting Fut8, FIG. 23E shows Cas9 targeting Bak1 and FIG. 23F shows CasΦ.12 targeting Bak1. FIG. 23G shows off-target analysis using unbiased guide-seq procedure, using CasΦ.12 and guides targeting human Fut8 in HEK293 cells. FIG. 23H shows off-target analysis using unbiased guide-seq procedure, using Cas9 and guides targeting human Fut8 in HEK293 cells.

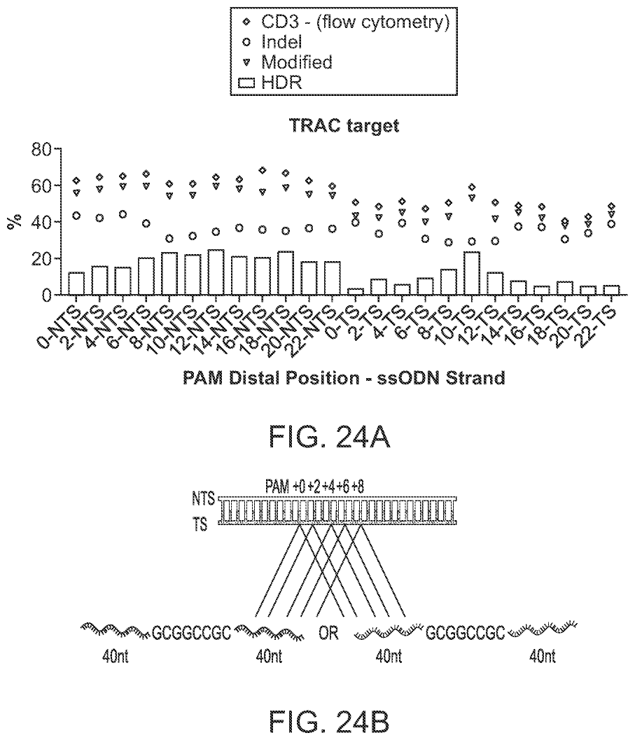

FIG. 24 illustrates CasΦ.12-mediated genome editing via homology directed repair (HDR). FIG. 24A shows CasΦ.12-mediated gene editing via the HDR pathway. FIG. 24B shows a schematic of the donor oligonucleotide

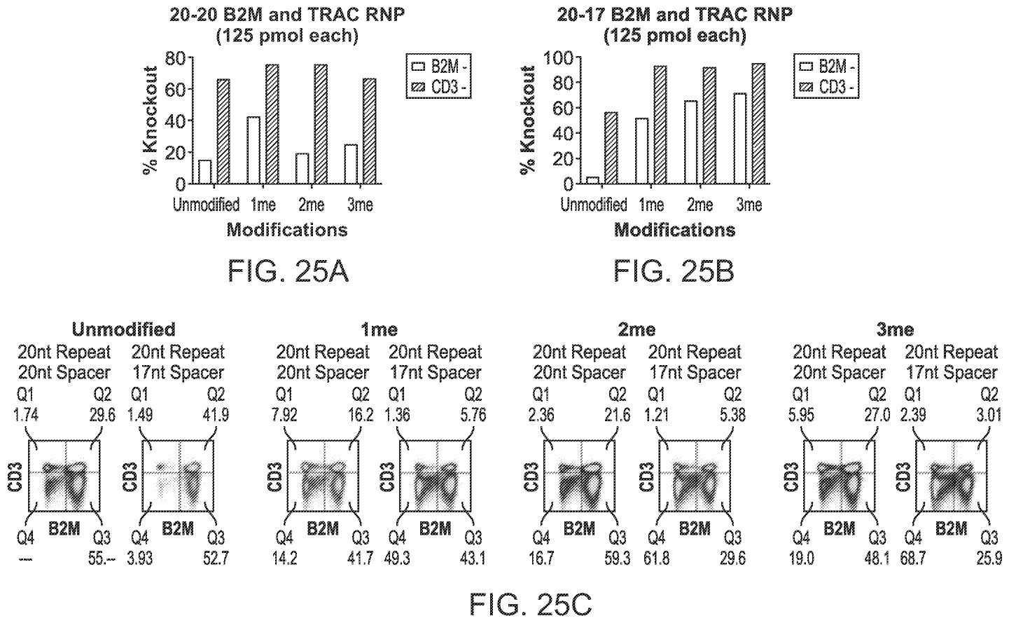

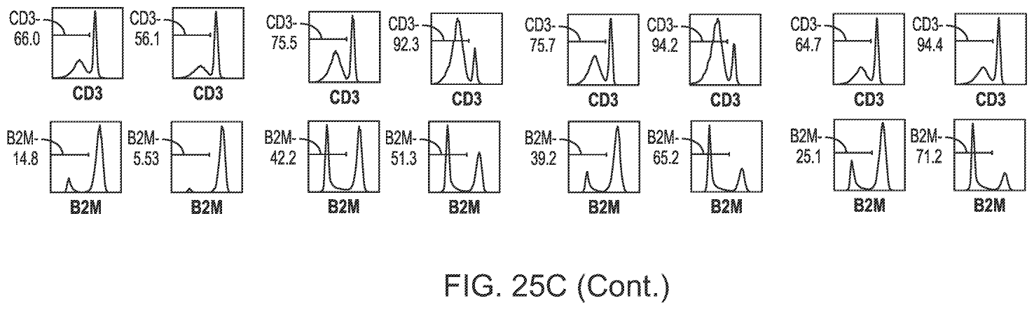

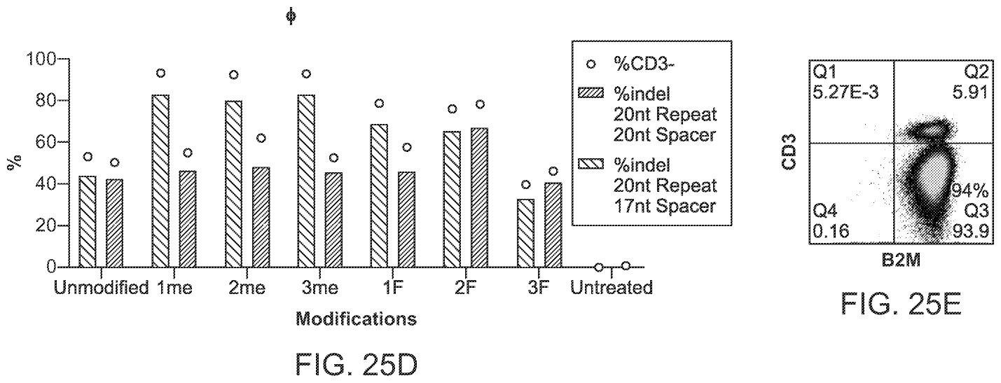

FIG. 25 illustrates the ability of CasΦ.12 to target multiple genes. FIG. 25A shows the percentage of B2M and TRAC knockout after CasΦ.12-mediated genome editing with gRNAs with a repeat length of 20 nucleotides and a spacer length of 20 nucleotides. FIG. 25B shows the percentage of B2M and TRAC knockout after CasΦ.12-mediated genome editing with gRNAs with a repeat length of 20 nucleotides and a spacer length of 17 nucleotides. FIG. 25C shows corresponding flow cytometry panels for B2M and TRAC knockout with different gRNAs. FIG. 25D shows the percentage of TRAC knockout after CasΦ.12-mediated genome editing with modified gRNAs of different spacer lengths (repeat length of 20 nucleotides and a spacer length of 17 or 20 nucleotides). FIG. 25E shows a corresponding flow cytometry panel for TRAC knockout after CasΦ.12-mediated genome editing.

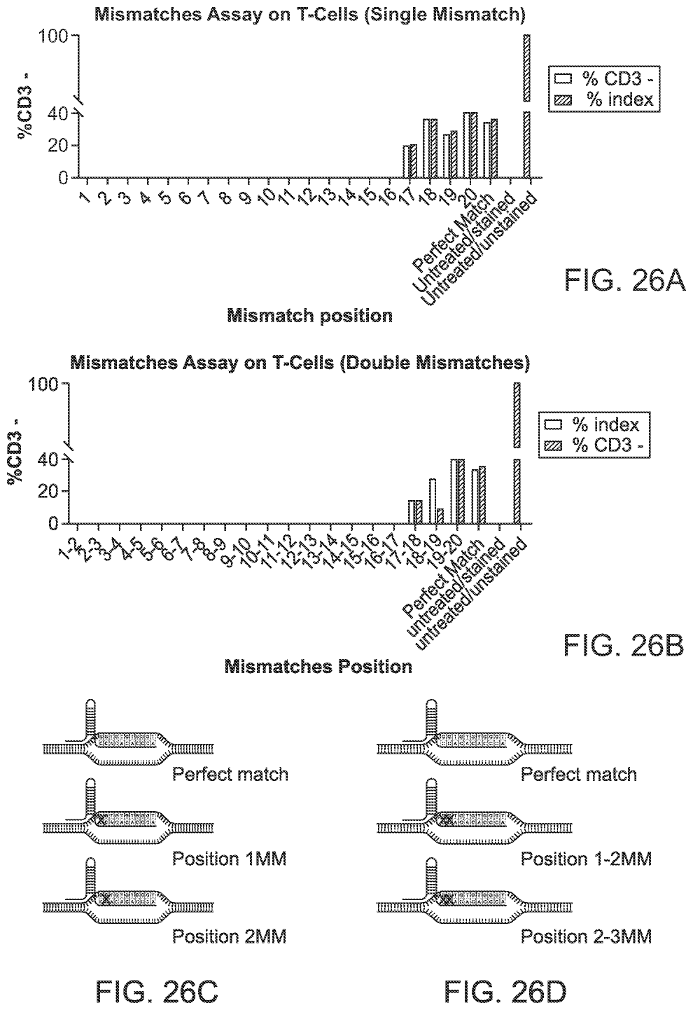

FIG. 26 illustrates the extended seed region of CasΦ.12. FIG. 26A and FIG. 26B show no indel mutations or CD3 knockout occurs when there is a single or double mismatch in the first 1-16 nucleotides from the 5′ end of the spacer. FIG. 26C and FIG. 26D provide schematics of the gRNAs with mismatches.

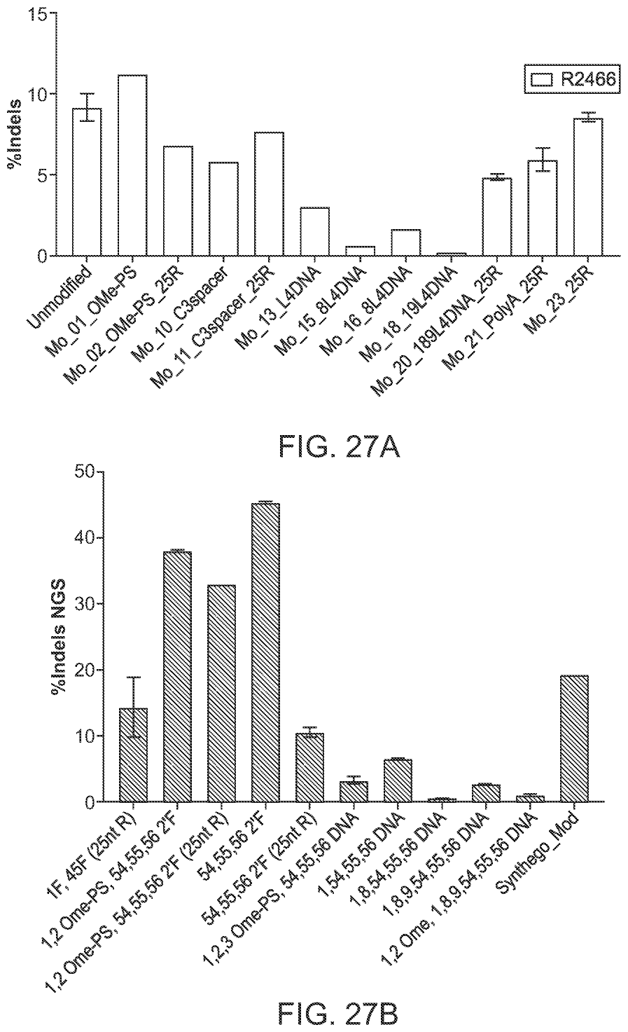

FIG. 27 illustrates the ability of CasΦ.12 to mediate genome editing in CHO cells with modified gRNAs.

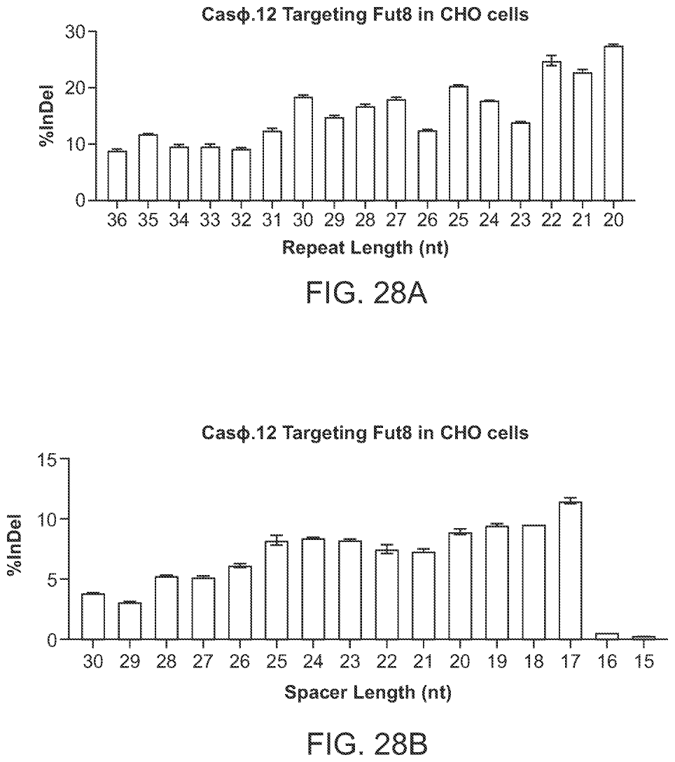

FIG. 28 illustrates the ability of CasΦ.12 to mediate genome editing with gRNAs with variations in repeat and spacer length. FIG. 28A shows the frequency of CasΦ.12-mediated indel mutations using gRNA of different repeat lengths. FIG. 28B shows the frequency of CasΦ.12-mediated indel mutations using gRNA of different spacer lengths.

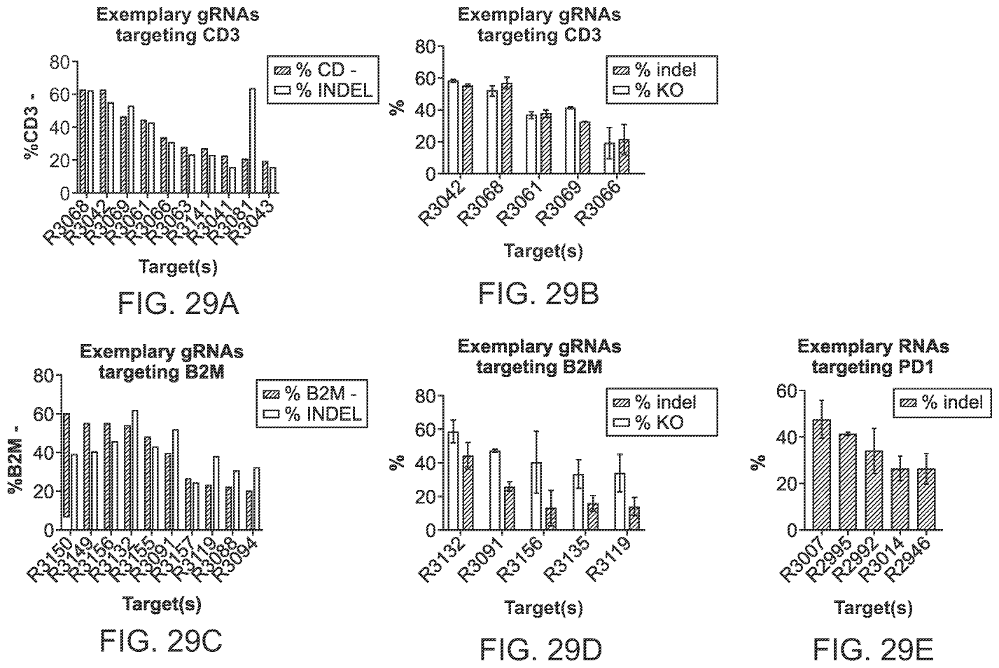

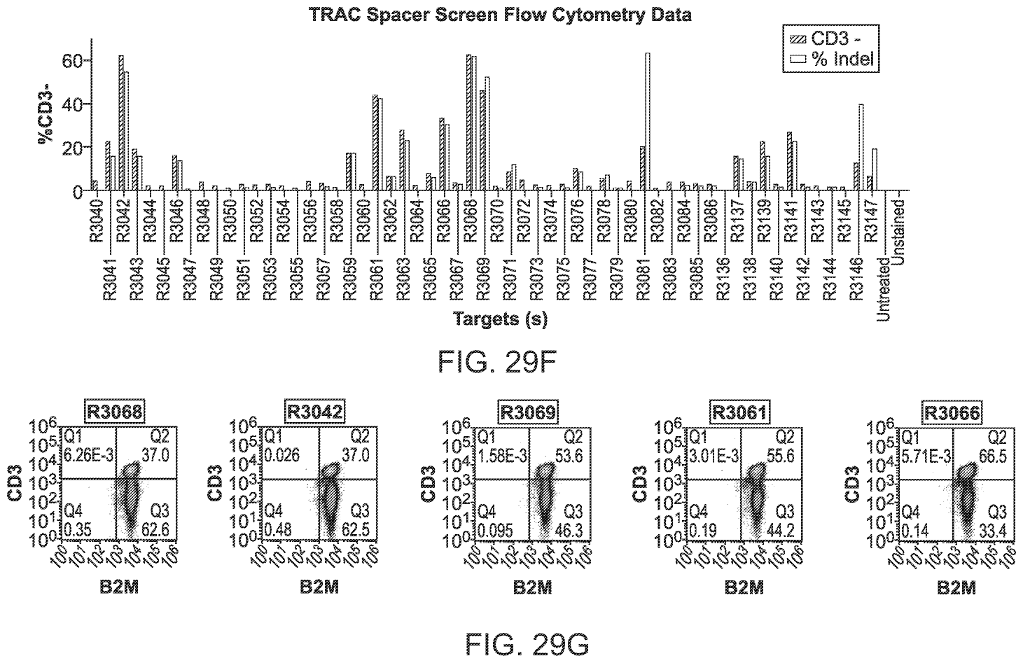

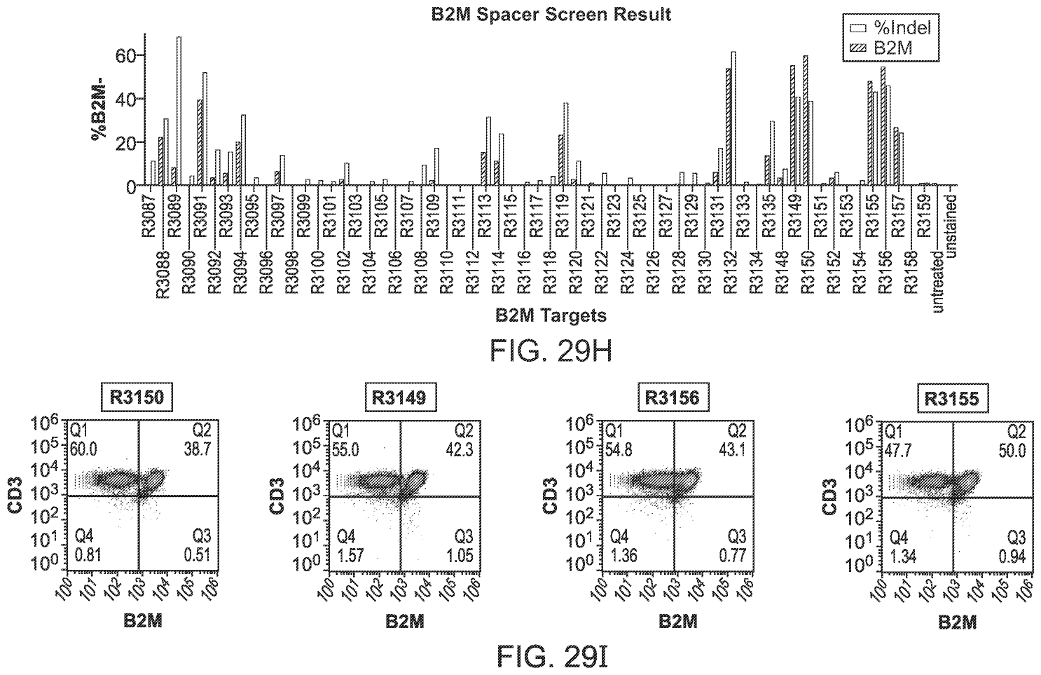

FIG. 29A-E illustrate exemplary gRNAs for targeting CD3, B2M and PD1 with CasΦ.12 in human primary T cells. FIG. 29F shows the screening of gRNAs targeting TRAC. FIG. 29H shows the screening of gRNAs targeting B2M. FIG. 29G and FIG. 29I show flow cytometry panels of exemplary gRNAs targeting TRAC and B2M, respectively.

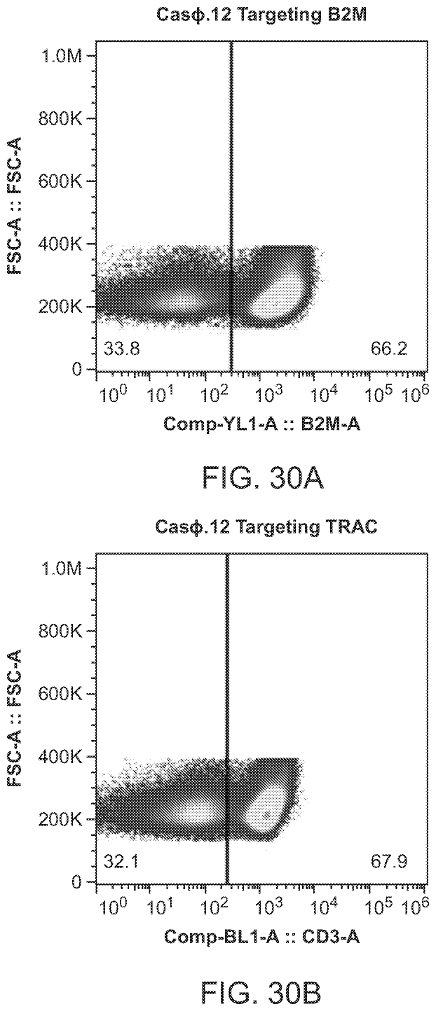

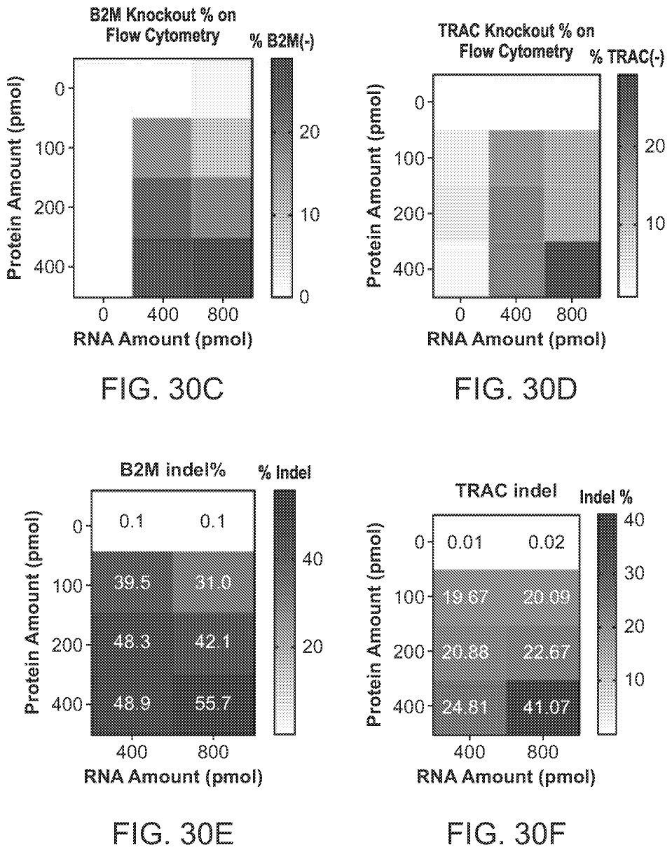

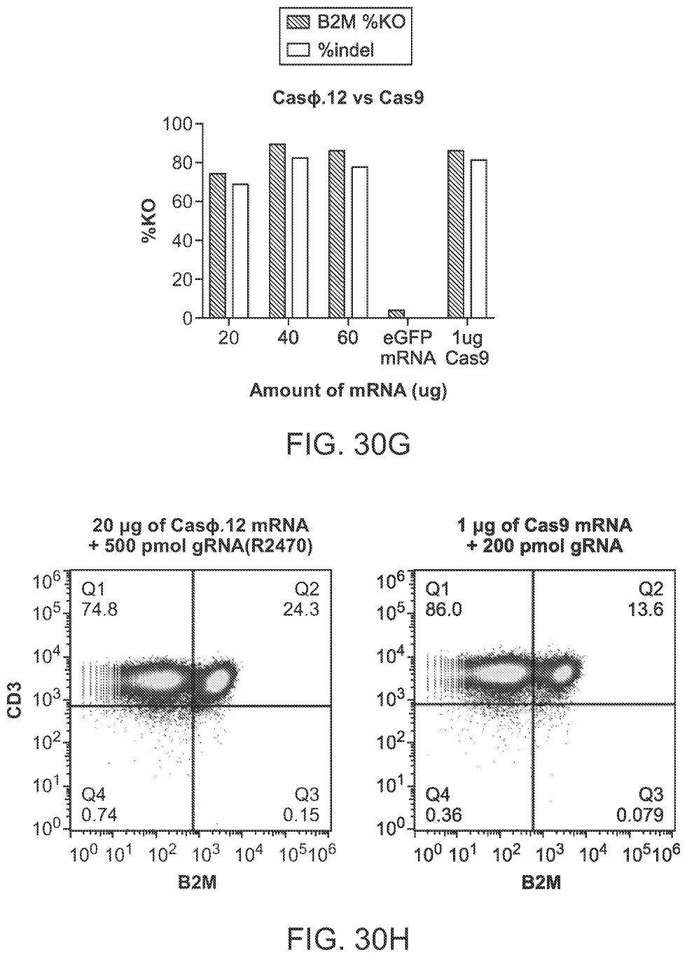

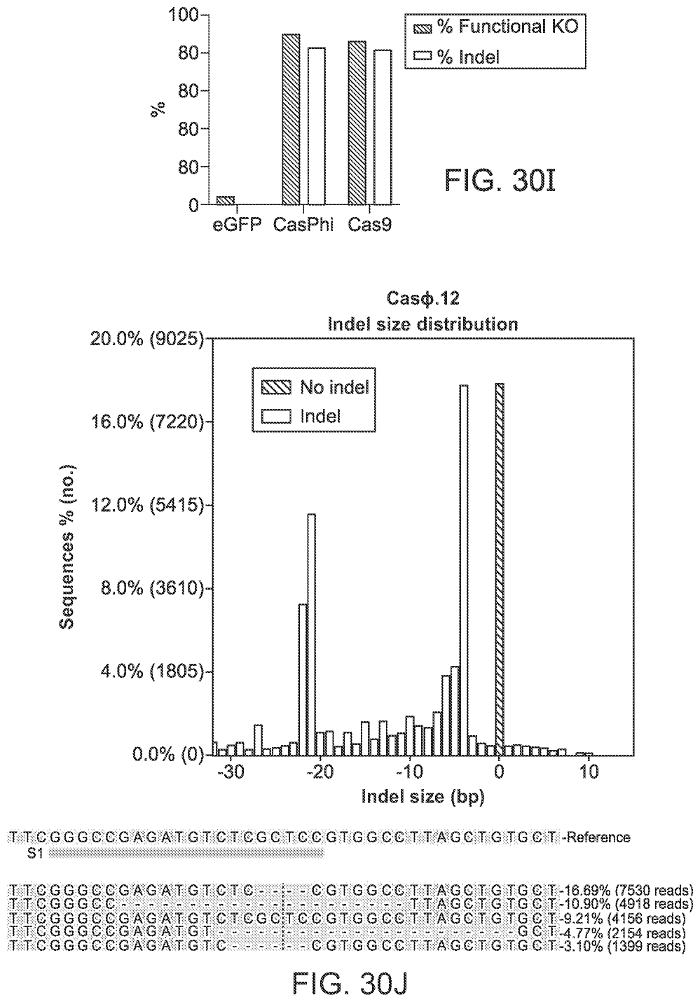

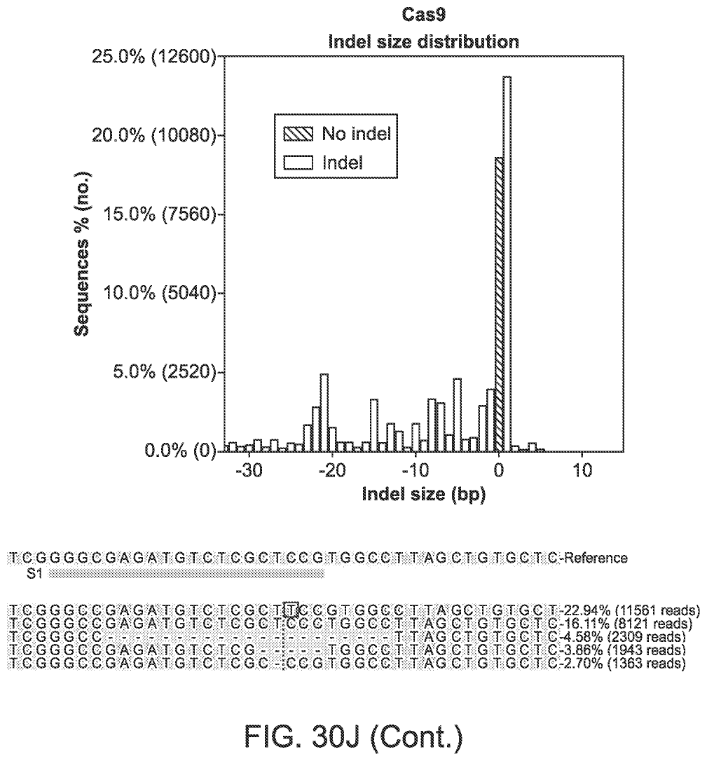

FIG. 30 illustrates delivery of CasΦ.12 RNPs or CasΦ.12 mRNA both lead to efficient genome editing. FIG. 30A and FIG. 30B show flow cytometry panels of CasΦ.12 RNP complexes targeting B2M and TRAC in T cells, and are quantified in FIG. 30C and FIG. 30D. FIG. 30E and FIG. 30F show the quantification of indels detected by sequence analysis with delivery of CasΦ.12 RNPs. FIG. 30G and FIG. 30I show the frequency of indel mutations after delivery of CasΦ.12 mRNA and the quantification of B2M knockout cells shown in FIG. 30H is an exemplary FACS panel for two data points in FIG. 30G. FIG. 30J shows the distribution of the size of indel mutations induced by CasΦ.12 or Cas9.

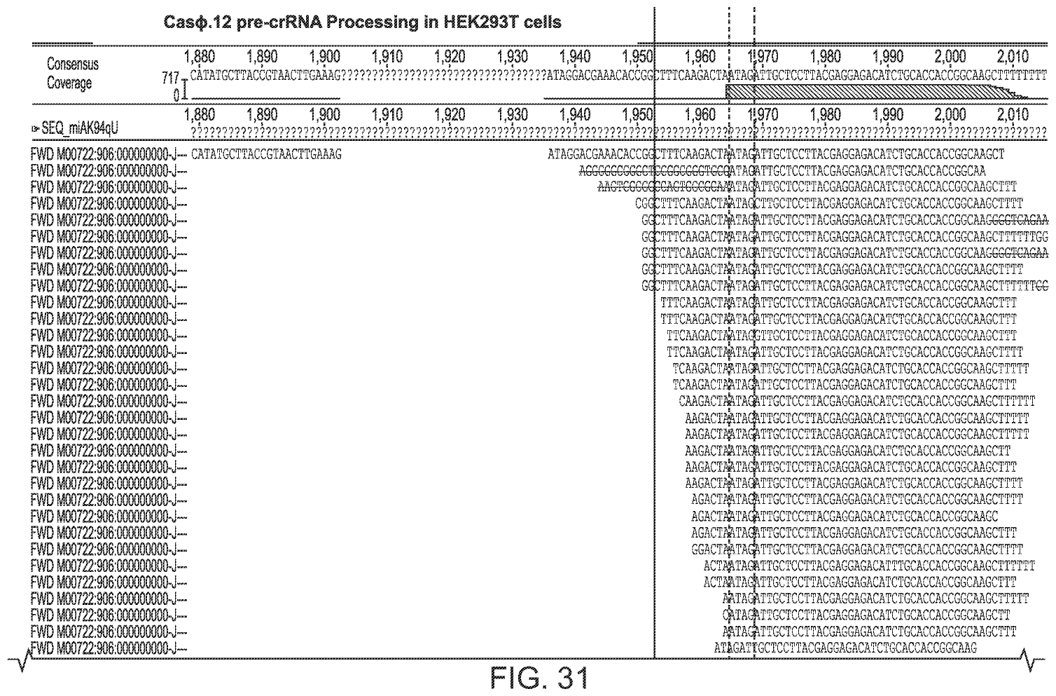

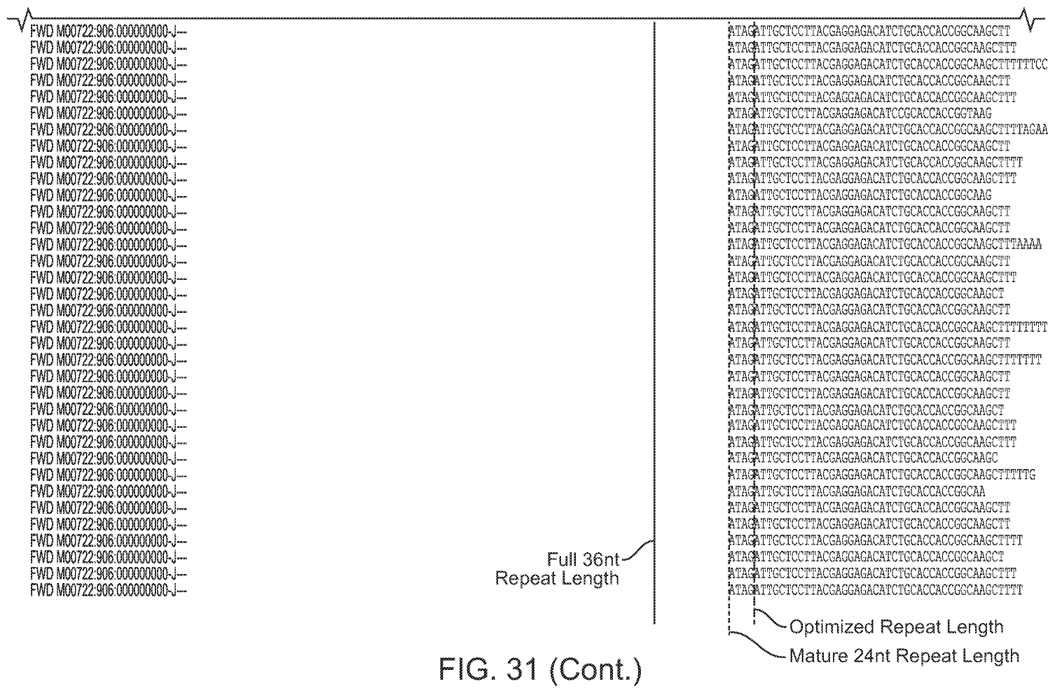

FIG. 31 illustrates CasΦ.12 can process its own guide RNA in mammalian cells.

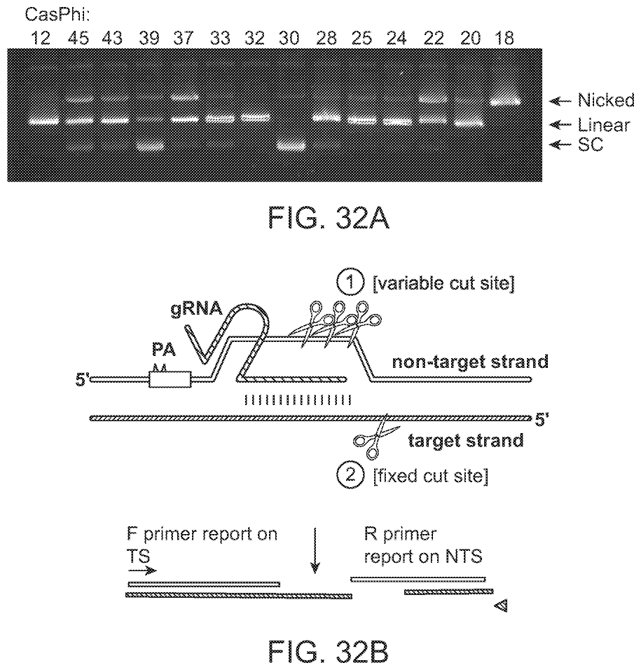

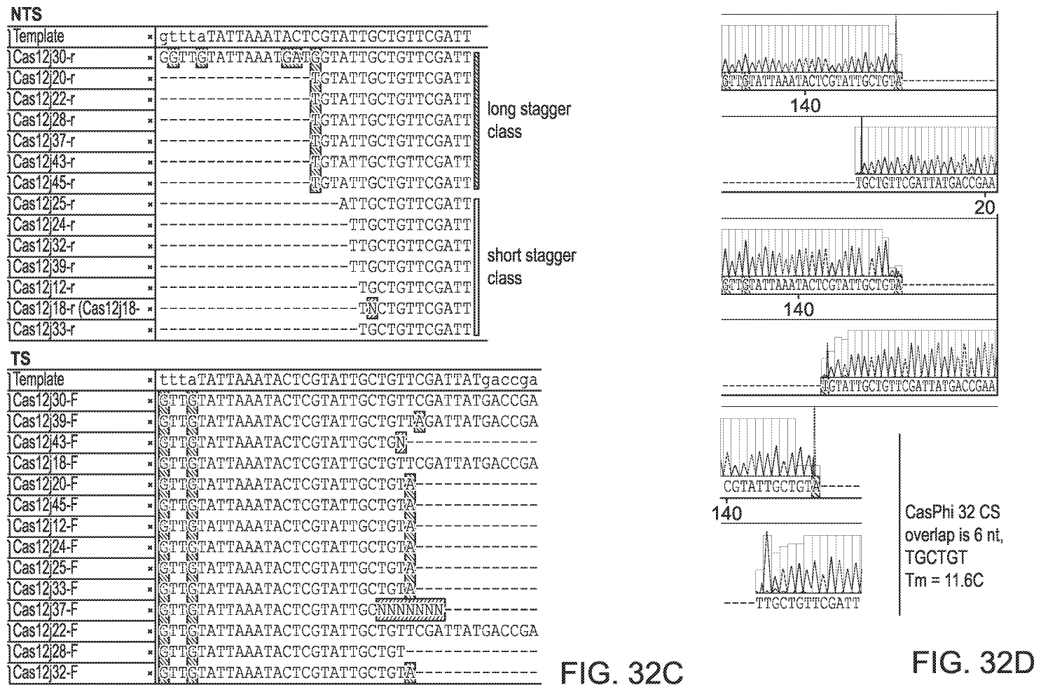

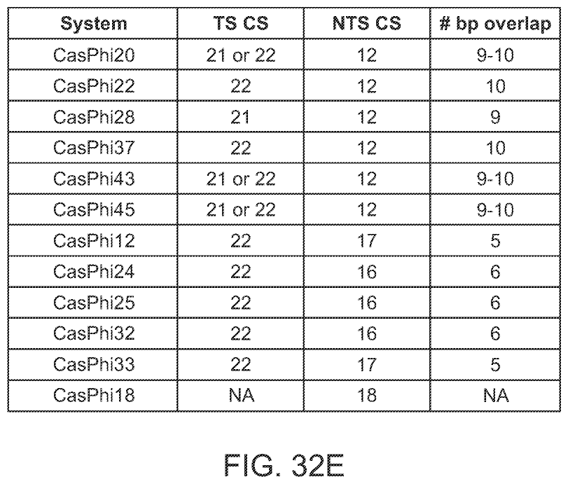

FIG. 32 illustrates CasΦ polypeptide-induced cleavage patterns. FIG. 32A, shows CasΦ polypeptides generated nicked and linearized plasmid DNA. FIG. 32B shows a schematic of the cut sites on the target and non-target strand. FIG. 32C shows sequence analysis of the non-target stand target strand and is represented in FIG. 32D. FIG. 32E shows a table of cut sites and overhangs of the different CasΦ polypeptides.

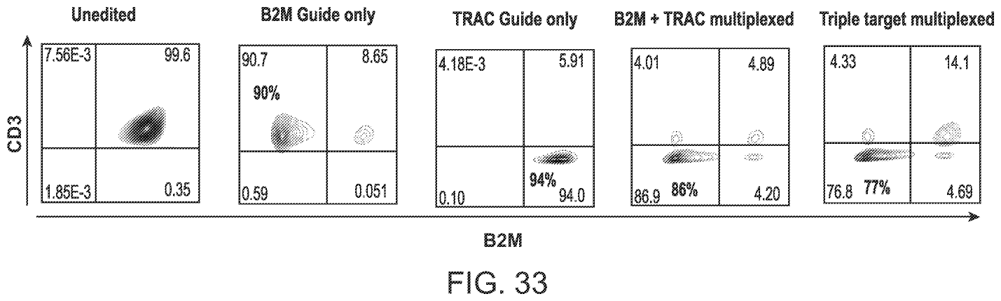

FIG. 33 illustrates the ability of CasΦ RNP complexes to knockout multiple genes simultaneously. T cells were nucleofected with RNP complexes of CasΦ.12 and gRNAs targeting B2M, TRAC or PDCD1 and the percentage knockout was measured using flow cytometry.

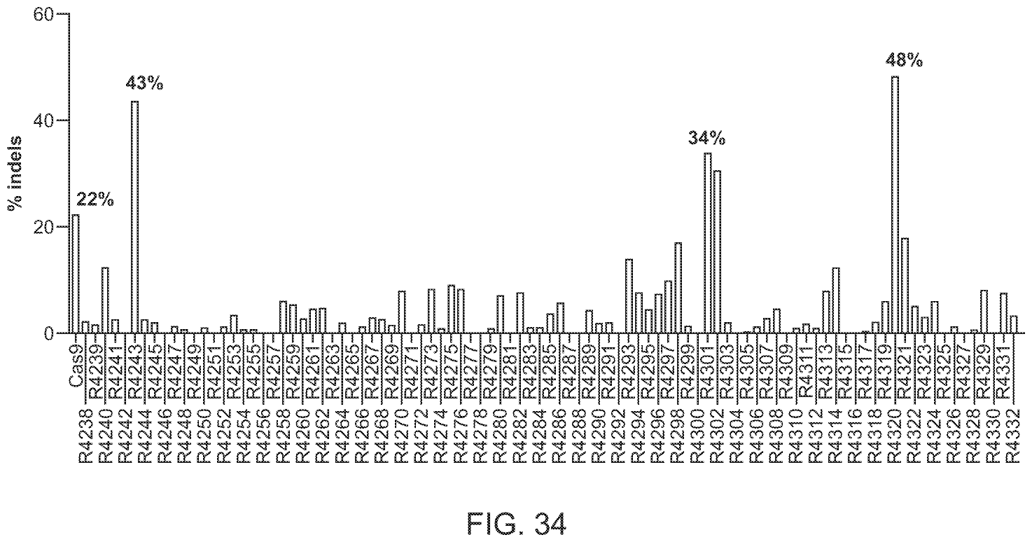

FIG. 34 illustrates the ability of CasΦ.12 RNP complexes to mediate high efficiency genome editing of PCKS9 in mouse Hepa1-6 cells. 95 CasΦ gRNAs were used along with Cas9, as a control. CasΦ.12 RNP complexes induced a maximum indel frequency of 48%, whereas Cas9 RNP complexed induced a maximum indel frequency of 22%.

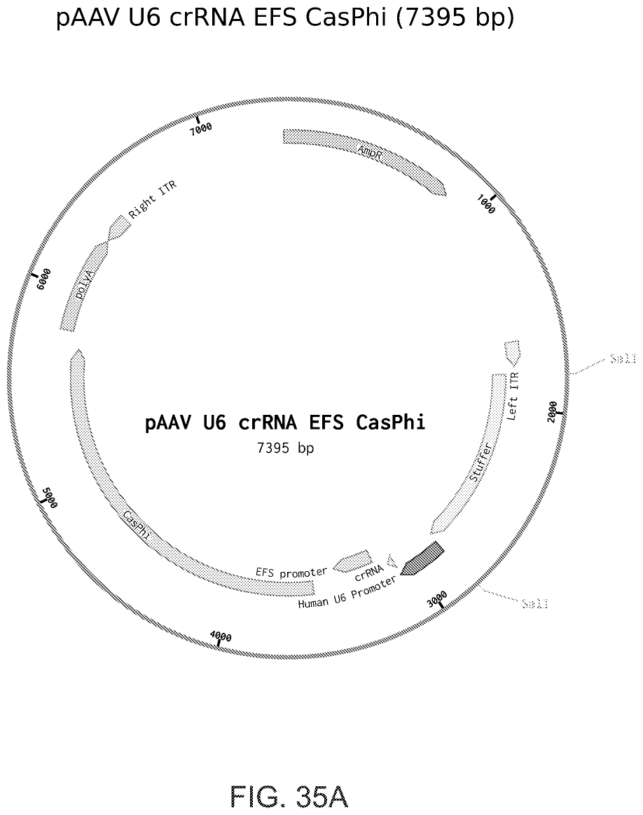

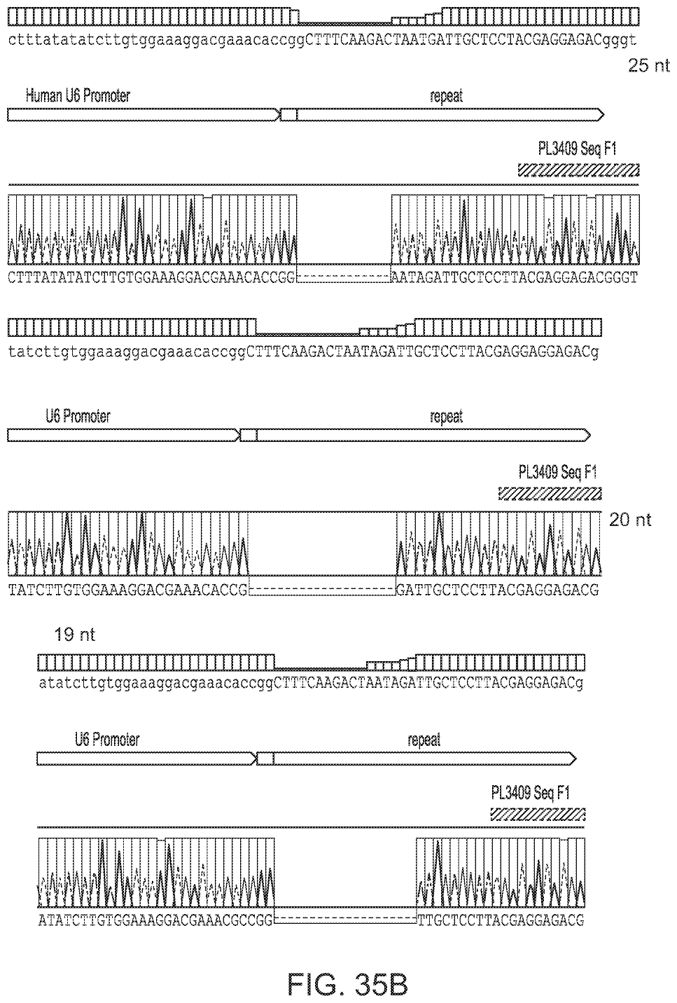

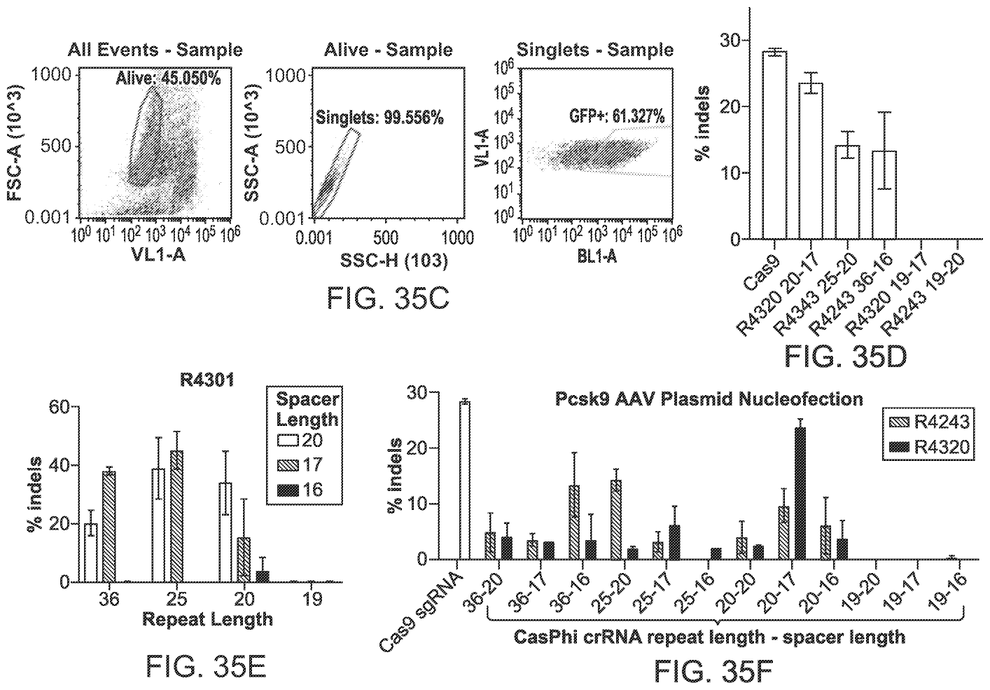

FIG. 35 illustrates the ability of a CasΦ.12 all-in-one vector to mediate genome editing in Hepa1-6 mouse hepatoma cells. FIG. 35A shows a plasmid map of the AAV encoding the CasΦ polypeptide sequence and gRNA sequence. FIG. 35B illustrates repeat truncations. FIG. 35C shows efficient transfection with AAV. FIG. 35D shows the frequency of CasΦ.12 induced indel mutations. FIG. 35E and FIG. 35F show the frequency of CasΦ.12 induced indel mutations with different gRNA containing repeat and spacer sequences of different lengths.

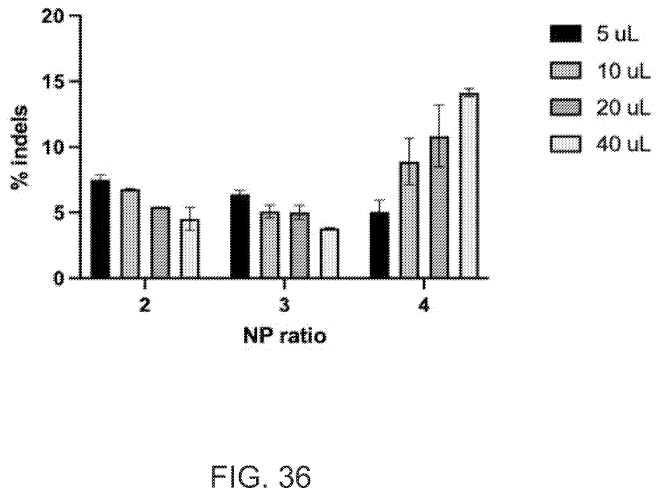

FIG. 36 illustrates the optimization of LNP delivery of mRNA encoding CasΦ and gRNA. A range of N/P ratios were tested and the frequency of indel mutations was determined.

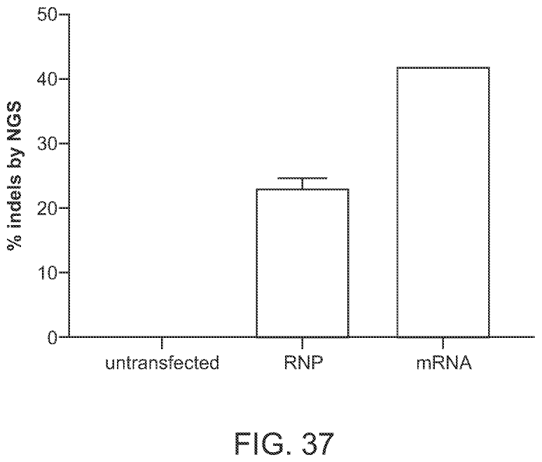

FIG. 37 illustrates CasΦ-mediated genome editing of CD34+ hematopoietic stem cells. Cells were nucleofected with either RNP complexes containing CasΦ.12 polypeptides and a B2M-targeting guide, or a mixture of CasΦ.12 mRNA and B2M-targeting guide and the frequency of indel mutations was determined.

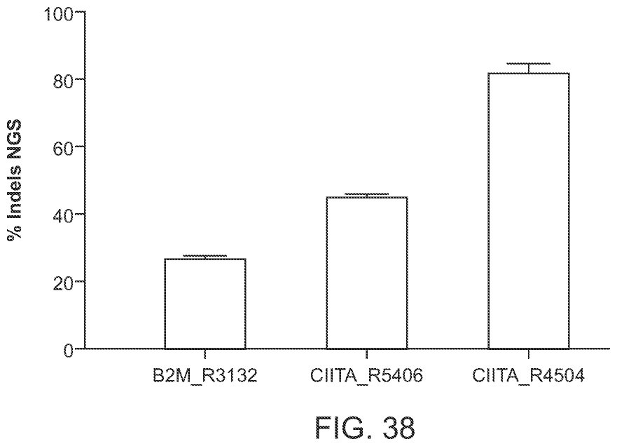

FIG. 38 illustrates CasΦ-mediated genome editing of induced pluripotent stem cells. Cells were nucleofected with RNP complexes (CasΦ.12 polypeptides and gRNAs targeting either the B2M locus or targeting a CIITA locus) and the frequency of indel mutations was determined.

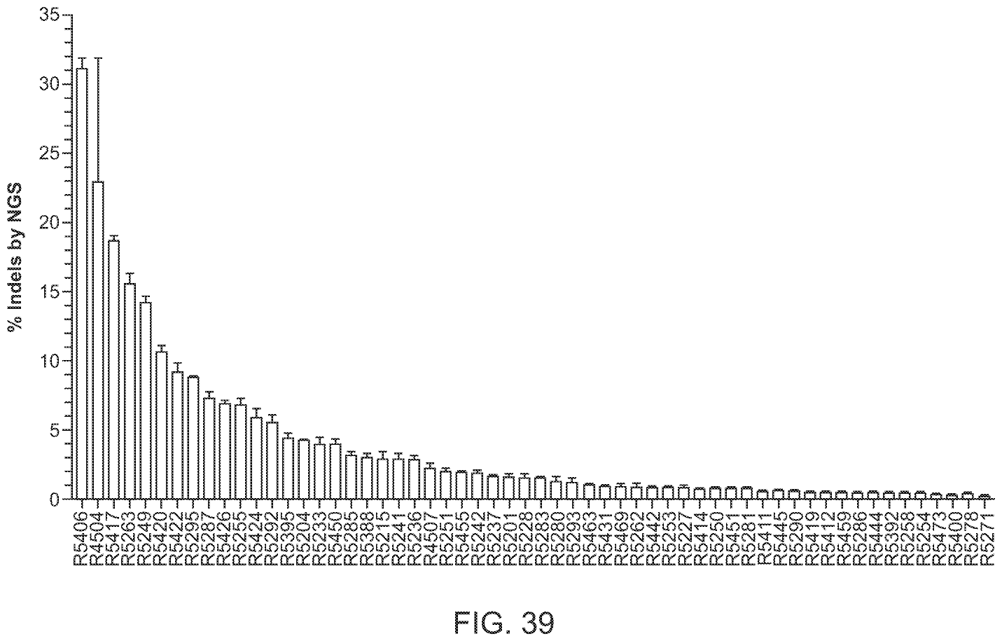

FIG. 39 illustrates CasΦ-mediated genome editing of the CIITA locus in K562 cells. Cells were nucleofected with RNP complexes (CasΦ polypeptides and gRNAs targeting CIITA) and the frequency of indel mutations was determined by NGS.

### DETAILED DESCRIPTION

The present disclosure provides methods, compositions, systems, and kits comprising programmable CasΦ nucleases. An illustrative composition comprises a programmable CasΦ nuclease or a nucleic acid encoding the programmable CasΦ nuclease, wherein the programmable CasΦ nuclease comprises at least 85% sequence identity to a sequence selected from the group consisting of SEQ ID NOs: 1 to 47 and SEQ ID NO. 105. In some embodiments, the composition further comprises a guide nucleic acid or a nucleic acid encoding the guide nucleic acid, wherein the guide nucleic acid comprises a region comprising a nucleotide sequence that is complementary to a target nucleic acid sequence and an additional region, wherein the region and the additional region are heterologous to each other. As used herein, the term “heterologous” may be used to describe or indicate that a first sequence is different from a second sequence and do not naturally occur together. As used herein, the term “heterologous” may be used to describe that a first moiety (e.g., a first sequence) is different from a second moiety (e.g., a second sequence) and, as such, the two moieties do not naturally occur together and are engineered to be a part of one entity. For example, a guide nucleic acid sequence comprising a region and an additional region that are heterologous to each other may indicate that the guide nucleic acid sequence is engineered to include the region and the additional region. The programmable CasΦ nuclease and the guide nucleic acid may be complexed together in a ribonucleoprotein complex. Alternatively, compositions consistent with the present disclosure include nucleic acids encoding for the programmable CasΦ nuclease and the guide nucleic acid. In some embodiments, the guide nucleic acid comprises a sequence with at least about 85% sequence identity to a sequence selected from the group consisting of SEQ ID NOs: 48 to 86. In some embodiments, the programmable CasΦ nuclease is SEQ ID NO: 12 or SEQ ID NO: 105. In some embodiments, the programmable CasΦ nuclease comprises nickase activity. In some embodiments, the programmable CasΦ nuclease comprises double-strand cleavage activity. As used herein, CasΦ may be referred to as Cas12j or Cas14u.

Also disclosed herein are compositions, methods, and systems for modifying a target nucleic acid sequence. An illustrative method for modifying a target nucleic acid sequence comprises contacting a target nucleic acid sequence with a programmable CasΦ nuclease comprising at least 85% sequence identity to a sequence selected from the group consisting of SEQ ID NOs: 1 to 47 and SEQ ID NO. 105, and a guide nucleic acid, wherein the programmable CasΦ nuclease cleaves the target nucleic acid sequence, thereby modifying the target nucleic acid sequence. In some embodiments, the programmable CasΦ nuclease introduces a double-stranded break in the target nucleic acid. In some embodiments, the programmable CasΦ nuclease introduces a single-stranded break.

Also disclosed herein are compositions, methods, and systems for modifying a target nucleic acid sequence comprising use of two or more programmable CasΦ nickases. An illustrative method for introducing a break in a target nucleic acid comprises contacting the target nucleic acid with: (a) a first guide nucleic acid comprising a region that binds to a first programmable nickase comprising at least 85% sequence identity to a sequence selected from the group consisting of SEQ ID NOs: 1 to 47 and SEQ ID NO. 105; and (b) a second guide nucleic acid comprising a region that binds to a second programmable nickase comprising at least 85% sequence identity to a sequence selected from the group consisting of SEQ ID NOs: 1 to 47 and SEQ ID NO. 105, wherein the first guide nucleic acid comprises an additional region that binds to the target nucleic acid and wherein the second guide nucleic acid comprises an additional region that binds to the target nucleic acid and wherein the additional region of the first guide nucleic acid and the additional region of the second guide nucleic acid bind opposing strands of the target nucleic acid.

Also disclosed herein are compositions, methods, and systems for detecting a target nucleic acid in a sample. An illustrative method for detecting a target nucleic acid in a sample comprises contacting the sample comprising the target nucleic acid with (a) a programmable CasΦ nuclease comprising at least 85% sequence identity to a sequence selected from the group consisting of SEQ ID NOs: 1 to 47 and SEQ ID NO. 105; (b) a guide RNA comprising a region that binds to the programmable CasΦ nuclease and an additional region that binds to the target nucleic acid; and (c) a labeled, single stranded DNA reporter that does not bind the guide RNA; cleaving the labeled single stranded DNA reporter by the programmable CasΦ nuclease to release a detectable label; and detecting the target nucleic acid by measuring a signal from the detectable label.

Also disclosed herein are compositions, methods, and systems for modulating transcription of a gene in a cell. An illustrative method of modulating transcription of a gene in a cell comprises introducing into a cell comprising a target nucleic acid sequence: (i) a fusion polypeptide or a nucleic acid encoding the fusion polypeptide, wherein the fusion polypeptide comprises: (a) a dCasΦ polypeptide comprising at least 85% sequence identity to a sequence selected from the group consisting of SEQ ID NOs: 1 to 47 and SEQ ID NO. 105, wherein the dCasΦ polypeptide is enzymatically inactive; and (b) a polypeptide comprising transcriptional regulation activity; and (ii) a guide nucleic acid, or a nucleic acid comprising a nucleotide sequence encoding the guide nucleic acid, wherein the guide nucleic acid comprises a region that binds to the dCasΦ polypeptide and an additional region that binds to the target nucleic acid; wherein transcription of the gene is modulated through the fusion polypeptide acting on the target nucleic acid sequence.

Also disclosed is use of a programmable CasΦ nuclease to modify a target nucleic acid sequence according to any of the methods described herein. Also disclosed is use of a first programmable nickase and a second programmable nickase to introduce a break in a target nucleic acid according to any of the methods described herein. Also disclosed is use of a programmable CasΦ nuclease to detect a target nucleic acid in a sample according to any of the methods described herein. Also disclosed is use of a dCasΦ polypeptide to modulate transcription of a gene in a cell according to any of the methods described herein.

### Programmable Nucleases

The present disclosure provides methods and compositions comprising programmable nucleases. The programmable nucleases can be complexed with a guide nucleic acid of the disclosure for targeting a target nucleic acid for detection, editing, modification, or regulation of the target nucleic acid.

The programmable nuclease can be used for detecting a target nucleic acid. For example, in certain embodiments, when the programmable nuclease is complexed with the guide nucleic acid and the target nucleic acid hybridizes to the guide nucleic acid, trans-cleavage of a single stranded DNA (ssDNA), such as an ssDNA reporter, by the programmable nuclease is activated. Detection of trans-cleavage of ssDNA can be used to determine a target nucleic acid in a sample.

The programmable nuclease can be used for editing or modifying a target nucleic acid, for example, by site-specific cleavage of a target sequence, donor nucleic acid insertion, or a combination thereof.

The programmable nuclease can be used for gene regulation of a target nucleic acid, for example, using a catalytically inactive programmable nuclease in combination with a polypeptide comprising gene regulation activity.

In some embodiments, the programmable nuclease is a programmable nuclease comprising site-specific nucleic acid cleavage activity. In some embodiments, the programmable nuclease is a programmable nuclease comprising double-strand DNA cleavage activity. In some embodiments, the programmable nuclease is a programmable nickase. In some embodiments, the programmable nuclease is a programmable DNA nickase. In some embodiments, the programmable nuclease is a programmable nuclease comprising a catalytically inactive nuclease domain. In some embodiments, the programmable nuclease comprising a catalytically inactive nuclease domain can include at least 1, at least 2, at least 3, at least 4, or at least 5 mutations relative to a wild type nuclease domain. Said mutations may be present within the cleaving or active site of the nuclease.

In some embodiments, the programmable nuclease is a programmable DNA nuclease. In some embodiments, the programmable nuclease is a Type V CRISPR/Cas enzyme, wherein a Type V CRISPR/Cas enzyme comprises a single active site or catalytic domain in a single RuvC domain. The RuvC domain is typically near the C-terminus of the enzyme. A single RuvC domain may comprise RuvC subdomains, for example RuvCI, RuvCII and RuvCIII. As used herein a “Type V CRISPR/Cas enzyme” or “Type V cas nuclease” or “Type V cas effector” may be used to describe a family of enzymes or a member thereof having diverse N-terminal structures and often comprising a conserved single catalytic RuvC-like endonuclease domain that is C-terminal of the N-terminal structures, derived from the TnpB protein encoded by autonomous or non-autonomous transposons. The terms “RuvC domain” and “RuvC-like domain” are used interchangeably for Type V CRISPR/Cas enzymes, Type V cas nucleases and Type V cas effectors. In some embodiments, the Type V CRISPR/Cas enzyme is a CasΦ nuclease. A CasΦ polypeptide can function as an endonuclease that catalyzes cleavage at a specific sequence in a target nucleic acid. A programmable CasΦ nuclease of the present disclosure may have a single active site in a RuvC domain that is capable of catalyzing pre-crRNA processing and nicking or cleaving of nucleic acids. This compact catalytic site may render the programmable CasΦ nuclease especially advantageous for genome engineering and new functionalities for genome manipulation.

In some embodiments, the RuvC domain is a RuvC-like domain. Various RuvC-like domains are known in the art and are easily identified using online tools such as InterPro (https://www.ebi.ac.uk/interpro/). For example, a RuvC-like domain may be a domain which shares homology with a region of TnpB proteins of the IS605 and other related families of transposons, as described in review articles such as Shmakov et al. (Nature Reviews Microbiology volume 15, pages 169-182(2017)) and Koonin E. V. and Makarova K. S. (2019, Phil. Trans. R. Soc., B 374:20180087). In some embodiments, the RuvC-like domain shares homology with the transposase IS605, OrfB, C-terminal. A transposase IS605, OrfB, C-terminal is easily identified by the skilled person using bioinformatics tools, such as PFAM (Finn et al. (Nucleic Acids Res. 2014 Jan. 1; 42(Database issue): D222-D230); El-Gebali et al. (2019) Nucleic Acids Res. doi:10.1093/nar/gky995). PFAM is a database of protein families in which each entry is composed of a seed alignment which forms the basis to build a profile hidden Markov model (HMM) using the HMMER software (hmmer.org). It is readily accessible via pfam.xfam.org, maintained by EMBL-EBI, which easily allows an amino acid sequence to be analyzed against the current release of PFAM (e.g. version 33.1 from May 2020), but local builds can also be implemented using publicly- and freely-available database files and tools. A transposase IS605, OrfB, C-terminal is easily identified by the skilled person using the HMM PF07282. PF07282 is reproduced for reference in FIG. 11 (accession number PF07282.12). The skilled person would also be able to identify a RuvC domain, for example with the HMM PF18516, using the PFAM tool. PF18516 is reproduced for reference in FIG. 12 (accession number PF18516.2). In some embodiments, the programmable CasΦ nuclease comprises a RuvC-like domain which matches PFAM family PF07282 but does not match PFAM family PF18516, as assessed using the PFAM tool (e.g. using PFAM version 33.1, and the HMM accession numbers PF07282.12 and PF18516.2). PFAM searches should ideally be performed using an E-value cut-off set at 1.0.

In some embodiments, a programmable nuclease described herein—or a programmable nuclease and guide RNA combination described herein—has an editing efficiency of at least 1%, at least 2%, at least 3%, at least 4%, at least 5%, at least 6%, at least 7%, at least 8%, at least 9%, at least 10%, at least 11%, at least 12%, at least 13%, at least 14%, at least 15%, at least 16%, at least 17%, at least 18%, at least 19%, at least 20%, at least 21%, at least 22%, at least 23%, at least 24%, at least 25%, at least 26%, at least 27%, at least 28%, at least 29%, at least 30%, at least 31%, at least 32%, at least 33%, at least 34%, at least 35%, at least 36%, at least 37%, at least 38%, at least 39%, at least 40%, at least 41%, at least 42%, at least 43%, at least 44%, at least 45%, at least 46%, at least 47%, at least 48%, at least 49%, at least 50%, at least 51%, at least 52%, at least 53%, at least 54%, at least 55%, at least 56%, at least 57%, at least 58%, at least 59%, at least 60%, at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100%. In some embodiments, a programmable nuclease described herein—or a programmable nuclease and guide RNA combination described herein—has an editing efficiency of at least 20%. In some embodiments, a programmable nuclease described herein—or a programmable nuclease and guide RNA combination described herein—has an editing efficiency of at least 25%. In some embodiments, a programmable nuclease described herein—or a programmable nuclease and guide RNA combination described herein—has an editing efficiency of at least 30%. In some embodiments, a programmable nuclease described herein—or a programmable nuclease and guide RNA combination described herein—has an editing efficiency of at least 35%. In some embodiments, a programmable nuclease described herein—or a programmable nuclease and guide RNA combination described herein—has an editing efficiency of at least 40%. In some embodiments, a programmable nuclease described herein—or a programmable nuclease and guide RNA combination described herein—has an editing efficiency of at least 45%. In some embodiments, a programmable nuclease described herein—or a programmable nuclease and guide RNA combination described herein—has an editing efficiency of at least 50%. In some embodiments, a programmable nuclease described herein—or a programmable nuclease and guide RNA combination described herein—has an editing efficiency of at least 55%. In some embodiments, a programmable nuclease described herein—or a programmable nuclease and guide RNA combination described herein—has an editing efficiency of at least 60%. In some embodiments, a programmable nuclease described herein—or a programmable nuclease and guide RNA combination described herein—has an editing efficiency of at least 65%. In some embodiments, a programmable nuclease described herein—or a programmable nuclease and guide RNA combination described herein—has an editing efficiency of at least 70%. In some embodiments, a programmable nuclease described herein—or a programmable nuclease and guide RNA combination described herein—has an editing efficiency of at least 75%. In some embodiments, a programmable nuclease described herein—or a programmable nuclease and guide RNA combination described herein—has an editing efficiency of at least 80%. In some embodiments, a programmable nuclease described herein—or a programmable nuclease and guide RNA combination described herein—has an editing efficiency of at least 85%. In some, a programmable nuclease described herein—or a programmable nuclease and guide RNA combination described herein—has an editing efficiency of at least 90%. In some embodiments, a programmable nuclease described herein—or a programmable nuclease and guide RNA combination described herein—has an editing efficiency of at least 95%. In some embodiments, a programmable nuclease described herein—or a programmable nuclease and guide RNA combination described herein—has an editing efficiency of at least 100%. In some embodiments, a programmable nuclease described herein—or a programmable nuclease and guide RNA combination described herein—has an editing efficiency of 42%. In some embodiments, said editing efficiency is determined by analyzing the frequency of indel mutations in a nucleic acid or gene knockout.

In some embodiments, a programmable nuclease described herein has a primary amino acid sequence length of less than 1500 amino acids, less than 1450 amino acids, less than 1400 amino acids, less than 1350 amino acids, less than 1300 amino acids, less than 1250 amino acids, less than 1200 amino acids, less than 1150 amino acids, less than 1100 amino acids, less than 1050 amino acids, less than 1000 amino acids, less than 950 amino acids, less than 900 amino acids, less than 850 amino acids, or less than 800 amino acids.

In some examples, a programmable nuclease described herein is a Type V cas nuclease. In some examples, the Type V cas nuclease, or a composition comprising the Type V cas nuclease, has an editing efficiency of at least 20%. In some examples, the Type V cas nuclease, or a composition comprising the Type V cas nuclease, has an editing efficiency of at least 25%. In some examples, the Type V cas nuclease, or a composition comprising the Type V cas nuclease, has an editing efficiency of at least 30%. In some examples, the Type V cas nuclease, or a composition comprising the Type V cas nuclease, has an editing efficiency of at least 35%. In some examples, the Type V cas nuclease, or a composition comprising the Type V cas nuclease, has an editing efficiency of at least 40%. In some examples, the Type V cas nuclease, or a composition comprising the Type V cas nuclease, has an editing efficiency of at least 45%. In some examples, the Type V cas nuclease, or a composition comprising the Type V cas nuclease, has an editing efficiency of at least 50%. In some examples, the Type V cas nuclease, or a composition comprising the Type V cas nuclease, has an editing efficiency of at least 55%. In some examples, the Type V cas nuclease, or a composition comprising the Type V cas nuclease, has an editing efficiency of at least 60%. In some examples, the Type V cas nuclease, or a composition comprising the Type V cas nuclease, has an editing efficiency of at least 65%. In some examples, the Type V cas nuclease, or a composition comprising the Type V cas nuclease, has an editing efficiency of at least 70%. In some examples, the Type V cas nuclease, or a composition comprising the Type V cas nuclease, has an editing efficiency of at least 75%. In some examples, the Type V cas nuclease, or a composition comprising the Type V cas nuclease, has an editing efficiency of at least 80%. In some examples, the Type V cas nuclease, or a composition comprising the Type V cas nuclease, has an editing efficiency of at least 85%. In some examples, the Type V cas nuclease, or a composition comprising the Type V cas nuclease, has an editing efficiency of at least 90%. In some examples, the Type V cas nuclease, or a composition comprising the Type V cas nuclease, has an editing efficiency of at least 95%. In some examples, the Type V cas nuclease, or a composition comprising the Type V cas nuclease, has an editing efficiency of 100%.

In some examples, a programmable nuclease described herein has a primary amino acid sequence length of less than 850 amino acids. In some examples, the programmable nuclease having a primary amino acid sequence length of less than 850 amino acids has an editing efficiency of at least 20%. In some examples, the programmable nuclease having a primary amino acid sequence length of less than 850 amino acids has an editing efficiency of at least 25%. In some examples, the programmable nuclease having a primary amino acid sequence length of less than 850 amino acids has an editing efficiency of at least 30%. In some examples, the programmable nuclease having a primary amino acid sequence length of less than 850 amino acids has an editing efficiency of at least 35%. In some examples, the programmable nuclease having a primary amino acid sequence length of less than 850 amino acids has an editing efficiency of at least 40%. In some examples, the programmable nuclease having a primary amino acid sequence length of less than 850 amino acids has an editing efficiency of at least 45%. In some examples, the programmable nuclease having a primary amino acid sequence length of less than 850 amino acids has an editing efficiency of at least 50%. In some examples, the programmable nuclease having a primary amino acid sequence length of less than 850 amino acids has an editing efficiency of at least 55%. In some examples, the programmable nuclease having a primary amino acid sequence length of less than 850 amino acids has an editing efficiency of at least 60%. In some examples, the programmable nuclease having a primary amino acid sequence length of less than 850 amino acids has an editing efficiency of at least 65%. In some examples, the programmable nuclease having a primary amino acid sequence length of less than 850 amino acids has an editing efficiency of at least 70%. In some examples, the programmable nuclease having a primary amino acid sequence length of less than 850 amino acids has an editing efficiency of at least 7500. In some examples, the programmable nuclease having a primary amino acid sequence length of less than 850 amino acids has an editing efficiency of at least 80%. In some examples, the programmable nuclease having a primary amino acid sequence length of less than 850 amino acids has an editing efficiency of at least 8500. In some examples, the programmable nuclease having a primary amino acid sequence length of less than 850 amino acids has an editing efficiency of at least 90%. In some examples, the programmable nuclease having a primary amino acid sequence length of less than 850 amino acids has an editing efficiency of at least 950%. In some examples, the programmable nuclease having a primary amino acid sequence length of less than 850 amino acids has an editing efficiency of 100%.

TABLE 1 provides amino acid sequences of illustrative CasΦ polypeptides that can be used in compositions and methods of the disclosure.

TABLE 1
CasΦ Amino Acid Sequences

| | SEQ ID | |
|---|---|---|
| Name | NO | Amino Acid Sequence |
| CasΦ.1 | 1 | MADTPTLFTQFLRHHLPGQRFRKDILKQAGRILANKGEDATI |
| | | AFLRGKSEESPPDFQPPVKCPIIACSRPLTEWPIYQASVAIQGY |
| | | VYGQSLAEFEASDPGCSKDGLLGWFDKTGVCTDYFSVQGLN |
| | | LIFQNARKRYIGVQTKVTNRNEKRHKKLKRINAKRIAEGLPE |
| | | LTSDEPESALDETGHLIDPPGLNTNIYCYQQVSPKPLALSEVN |
| | | QLPTAYAGYSTSGDDPIQPMVTKDRLSISKGQPGYIPEHQRA |
| | | LLSQKKHRRMRGYGLKARALLVIVRIQDDWAVIDLRSLLRN |
| | | AYWRRIVQTKEPSTITKLLKLVTGDPVLDATRMVATFTYKPG |
| | | IVQVRSAKCLKNKQGSKLFSERYLNETVSVTSIDLGSNNLVA |
| | | VATYRLVNGNTPELLQRFTLPSHLVKDFERYKQAHDTLEDSI |
| | | QKTAVASLPQGQQTEIRMWSMYGFREAQERVCQELGLADG |
| | | SIPWNVMTATSTILTDLFLARGGDPKKCMFTSEPKKKKNSKQ |
| | | VLYKIRDRAWAKMYRTLLSKETREAWNKALWGLKRGSPDY |
| | | ARLSKRKEELARRCVNYTISTAEKRAQCGRTIVALEDLNIGFF |
| | | HGRGKQEPGWVGLFTRKKENRWLMQALHKAFLELAHHRG |
| | | YHVIEVNPAYTSQTCPVCRHCDPDNRDQHNREAFHCIGCGFR |
| | | GNADLDVATHNIAMVAITGESLKRARGSVASKTPQPLAAE |
| CasΦ.2 | 2 | MPKPAVESEFSKVLKKHFPGERFRSSYMKRGGKILAAQGEE |
| | | AVVAYLQGKSEEEPPNFQPPAKCHVVTKSRDFAEWPIMKAS |
| | | EAIQRYIYALSTTERAACKPGKSSESHAAWFAATGVSNHGYS |
| | | HVQGLNLIFDHTLGRYDGVLKKVQLRNEKARARLESINASR |
| | | ADEGLPEIKAEEEEVATNETGHLLQPPGINPSFYVYQTISPQA |
| | | YRPRDEIVLPPEYAGYVRDPNAPIPLGVVRNRCDIQKGCPGYI |
| | | PEWQREAGTAISPKTGKAVTVPGLSPKKNKRMRRYWRSEKE |
| | | KAQDALLVTVRIGTDWVVIDVRGLLRNARWRTIAPKDISLN |
| | | ALLDLFTGDPVIDVRRNIVTFTYTLDACGTYARKWTLKGKQ |
| | | TKATLDKLTATQTVALVAIDLGQTNPISAGISRVTQENGALQ |
| | | CEPLDRFTLPDDLLKDISAYRIAWDRNEEELRARSVEALPEA |
| | | QQAEVRALDGVSKETARTQLCADFGLDPKRLPWDKMSSNT |
| | | TFISEALLSNSVSRDQVFFTPAPKKGAKKKAPVEVMRKDRT |
| | | WARAYKPRLSVEAQKLKNEALWALKRTSPEYLKLSRRKEEL |
| | | CRRSINYVIEKTRRRTQCQIVIPVIEDLNVRFFHGSGKRLPGW |
| | | DNFFTAKKENRWFIQGLHKAFSDLRTHRSFYVFEVRPERTSIT |
| | | CPKCGHCEVGNRDGEAFQCLSCGKTCNADLDVATHNLTQV |
| | | ALTGKTMPKREEPRDAQGTAPARKTKKASKSKAPPAEREDQ |
| | | TPAQEPSQTS |
| CasΦ.3 | 3 | MYILEMADLKSEPSLLAKLLRDRFPGKYWLPKYWKLAEKKR |
| | | LTGGEEAACEYMADKQLDSPPPNFRPPARCVILAKSRPFEDW |
| | | PVHRVASKAQSFVIGLSEQGFAALRAAPPSTADARRDWLRS |
| | | HGASEDDLMALEAQLLETIMGNAISLHGGVLKKIDNANVKA |
| | | AKRLSGRNEARLNKGLQELPPEQEGSAYGADGLLVNPPGLN |
| | | LNIYCRKSCCPKPVKNTARFVGHYPGYLRDSDSILISGTMDR |
| | | LTIIEGMPGHIPAWQREQGLVKPGGRRRRLSGSESNMRQKVD |
| | | PSTGPRRSTRSGTVNRSNQRTGRNGDPLLVEIRMKEDWVLL |
| | | DARGLLRNLRWRESKRGLSCDHEDLSLSGLLALFSGDPVIDP |
| | | VRNEVVFLYGEGIIPVRSTKPVGTRQSKKLLERQASMGPLTLI |
| | | SCDLGQTNLIAGRASAISLTHGSLGVRSSVRIELDPEIIKSFERL |
| | | RKDADRLETEILTAAKETLSDEQRGEVNSHEKDSPQTAKASL |
| | | CRELGLHPPSLPWGQMGPSTTFIADMLISHGRDDDAFLSHGE |
| | | FPTLEKRKKFDKRFCLESRPLLSSETRKALNESLWEVKRTSSE |
| | | YARLSQRKKEMARRAVNFVVEISRRKTGLSNVIVNIEDLNVR |
| | | IFHGGGKQAPGWDGFFRPKSENRWFIQAIHKAFSDLAAHHGI |
| | | PVIESDPQRTSMTCPECGHCDSKNRNGVRFLCKGCGASMDA |
| | | DFDAACRNLERVALTGKPMPKPSTSCERLLSATTGKVCSDHS |
| | | LSHDAIEKAS |
| CasΦ.4 | 4 | MEKEITELTKIRREFPNKKFSSTDMKKAGKLLKAEGPDAVRD |
| | | FLNSCQEIIGDFKPPVKTNIVSISRPFEEWPVSMVGRAIQEYYF |
| | | SLTKEELESVHPGTSSEDHKSFFNITGLSNYNYTSVQGLNLIF |
| | | KNAKAIYDGTLVKANNKNKKLEKKFNEINHKRSLEGLPIITP |
| | | DFEEPFDENGHLNNPPGINRNIYGYQGCAAKVFVPSKHKMV |
| | | SLPKEYEGYNRDPNLSLAGFRNRLEIPEGEPGHVPWFQRMDI |
| | | PEGQIGHVNKIQRFNFVHGKNSGKVKFSDKTGRVKRYHHSK |
| | | YKDATKPYKFLEESKKVSALDSILAHITIGDDWVVFDIRGLYR |
| | | NVFYRELAQKGLTAVQLLDLFTGDPVIDPKKGVVTFSYKEG |
| | | VVPVFSQKIVPRFKSRDTLEKLTSQGPVALLSVDLGQNEPVA |
| | | ARVCSLKNINDKITLDNSCRISFLDDYKKQIKDYRDSLDELEI |
| | | KIRLEAINSLETNQQVEIRDLDVFSADRAKANTVDMFDIDPN |
| | | LISWDSMSDARVSTQISDLYLKNGGDESRVYFEINNKRIKRS |
| | | DYNISQLVRPKLSDSTRKNLNDSIWKLKRTSEEYLKLSKRKL |
| | | ELSRAVVNYTIRQSKLLSGINDIVIILEDLDVKKKFNGRGIRDI |
| | | GWDNFFSSRKENRWFIPAFHKAFSELSSNRGLCVIEVNPAWT |
| | | SATCPDCGFCSKENRDGINFTCRKCGVSYHADIDVATLNIAR |
| | | VAVLGKPMSGPADRERLGDTKKPRVARSRKTMKRKDISNST |
| | | VEAMVTA |
| CasΦ.5 | 5 | MDMLDTETNYATETPAQQQDYSPKPPKKAQRAPKGFSKKA |
| | | RPEKKPPKPITLFTQKHFSGVRFLKRVIRDASKILKLSESRTITF |
| | | LEQAIERDGSAPPDVTPPVHNTIMAVTRPFEEWPEVILSKALQ |
| | | KHCYALTKKIKIKTWPKKGPGKKCLAAWSARTKIPLIPGQVQ |
| | | ATNGLFDRIGSIYDGVEKKVTNRNANKKLEYDEAIKEGRNPA |
| | | VPEYETAYNIDGTLINKPGYNPNLYITQSRTPRLITEADRPLVE |
| | | KILWQMVEKKTQSRNQARRARLEKAAHLQGLPVPKFVPEK |
| | | VDRSQKIEIRIIDPLDKIEPYMPQDRMAIKASQDGHVPYWQRP |
| | | FLSKRRNRRVRAGWGKQVSSIQAWLTGALLVIVRLGNEAFL |
| | | ADIRGALRNAQWRKLLKPDATYQSLFNLFTGDPVVNTRTNH |
| | | LTMAYREGVVNIVKSRSFKGRQTREHLLTLLGQGKTVAGVS |
| | | FDLGQKHAAGLLAAHFGLGEDGNPVFTPIQACFLPQRYLDSL |
| | | TNYRNRYDALTLDMRRQSLLALTPAQQQEFADAQRDPGGQ |
| | | AKRACCLKLNLNPDEIRWDLVSGISTMISDLYIERGGDPRDV |
| | | HQQVETKPKGKRKSEIRILKIRDGKWAYDFRPKIADETRKAQ |
| | | REQLWKLQKASSEFERLSRYKINIARAIANWALQWGRELSG |
| | | CDIVIPVLEDLNVGSKFFDGKGKWLLGWDNRFTPKKENRWF |
| | | IKVLHKAVAELAPHRGVPVYEVMPHRTSMTCPACHYCHPTN |
| | | REGDRFECQSCHVVKNTDRDVAPYNILRVAVEGKTLDRWQ |
| | | AEKKPQAEPDRPMILIDNQES |
| CasΦ.6 | 6 | MDMLDTETNYATETPAQQQDYSPKPPKKAQRAPKGFSKKA |
| | | RPEKKPPKPITLFTQKHFSGVRFLKRVIRDASKILKLSESRTITF |
| | | LEQAIERDGSAPPDVTPPVHNTIMAVTRPFEEWPEVILSKALQ |
| | | KHCYALTKKIKIKTWPKKGPGKKCLAAWSARTKIPLIPGQVQ |
| | | ATNGLFDRIGSIYDGVEKKVTNRNANKKLEYDEAIKEGRNPA |
| | | VPEYETAYNIDGTLINKPGYNPNLYITQSRTPRLITEADRPLVE |
| | | KILWQMVEKKTQSRNQARRARLEKAAHLQGLPVPKFVPEK |
| | | VDRSQKIEIRIIDPLDKIEPYMPQDRMAIKASQDGHVPYWQRP |
| | | FLSKRRNRRVRAGWGKQVSSIQAWLTGALLVIVRLGNEAFL |
| | | ADIRGALRNAQWRKLLKPDATYQSLFNLFTGDPVVNTRTNH |
| | | LTMAYREGVVDIVKSRSFKGRQTREHLLTLLGQGKTVAGVS |
| | | FDLGQKHAAGLLAAHFGLGEDGNPVFTPIQACFLPQRYLDSL |
| | | TNYRNRYDALTLDMRRQSLLALTPAQQQEFADAQRDPGGQ |
| | | AKRACCLKLNLNPDEIRWDLVSGISTMISDLYIERGGDPRDV |
| | | HQQVETKPKGKRKSEIRILKIRDGKWAYDFRPKIADETRKAQ |
| | | REQLWKLQKASSEFERLSRYKINIARAIANWALQWGRELSG |
| | | CDIVIPVLEDLNVGSKFFDGKGKWLLGWDNRFTPKKENRWF |
| | | IKVLHKAVAELAPHKGVPVYEVMPHRTSMTCPACHYCHPTN |
| | | REGDRFECQSCHVVKNTDRDVAPYNILRVAVEGKTLDRWQ |
| | | AEKKPQAEPDRPMILIDNQES |
| CasΦ.7 | 7 | MSSLPTPLELLKQKHADLFKGLQFSSKDNKMAGKVLKKDGE |
| | | EAALAFLSERGVSRGELPNFRPPAKTLVVAQSRPFEEFPIYRV |
| | | SEAIQLYVYSLSVKELETVPSGSSTKKEHQRFFQDSSVPDFGY |
| | | TSVQGLNKIFGLARGIYLGVITRGENQLQKAKSKHEALNKKR |
| | | RASGEAETEFDPTPYEYMTPERKLAKPPGVNHSIMCYVDISV |
| | | DEFDFRNPDGIVLPSEYAGYCREINTAIEKGTVDRLGHLKGG |
| | | PGYIPGHQRKESTTEGPKINFRKGRIRRSYTALYAKRDSRRVR |
| | | QGKLALPSYRHHMMRLNSNAESAILAVIFFGKDWVVFDLRG |
| | | LLRNVRWRNLFVDGSTPSTLLGMFGDPVIDPKRGVVAFCYK |
| | | EQIVPVVSKSITKMVKAPELLNKLYLKSEDPLVLVAIDLGQT |
| | | NPVGVGVYRVMNASLDYEVVTRFALESELLREIESYRQRTN |
| | | AFEAQIRAETFDAMTSEEQEEITRVRAFSASKAKENVCHRFG |
| | | MPVDAVDWATMGSNTIHIAKWVMRHGDPSLVEVLEYRKDN |
| | | EIKLDKNGVPKKVKLTDKRIANLTSIRLRFSQETSKHYNDTM |
| | | WELRRKHPVYQKLSKSKADFSRRVVNSIIRRVNHLVPRARIV |
| | | FIIEDLKNLGKVFHGSGKRELGWDSYFEPKSENRWFIQVLHK |
| | | AFSETGKHKGYYIIECWPNWTSCTCPKCSCCDSENRHGEVFR |
| | | CLACGYTCNTDFGTAPDNLVKIATTGKGLPGPKKRCKGSSK |
| | | GKNPKIARSSETGVSVTESGAPKVKKSSPTQTSQSSSQSAP |
| CasΦ.8 | 8 | MNKIEKEKTPLAKLMNENFAGLRFPFAIIKQAGKKLLKEGEL |
| | | KTIEYMTGKGSIEPLPNFKPPVKCLIVAKRRDLKYFPICKASC |
| | | EIQSYVYSLNYKDFMDYFSTPMTSQKQHEEFFKKSGLNIEYQ |
| | | NVAGLNLIFNNVKNTYNGVILKVKNRNEKLKKKAIKNNYEF |
| | | EEIKTFNDDGCLINKPGINNVIYCFQSISPKILKNITHLPKEYND |
| | | YDCSVDRNIIQKYVSRLDIPESQPGHVPEWQRKLPEFNNTNN |
| | | PRRRRKWYSNGRNISKGYSVDQVNQAKIEDSLLAQIKIGED |
| | | WIILDIRGLLRDLNRRELISYKNKLTIKDVLGFFSDYPIIDIKKN |
| | | LVTFCYKEGVIQVVSQKSIGNKKSKQLLEKLIENKPIALVSID |
| | | LGQTNPVSVKISKLNKINNKISIESFTYRFLNEEILKEIEKYRK |
| | | DYDKLELKLINEA |
| CasΦ.9 | 9 | MDMLDTETNYATETPSQQQDYSPKPPKKDRRAPKGFSKKAR |
| | | PEKKPPKPITLFTQKHFSGVRFLKRVIRDASKILKLSESRTITFL |
| | | EQAIERDGSAPPDVTPPVHNTIMAVTRPFEEWPEVILSKALQK |
| | | HCYALTKKIKIKTWPKKGPGKKCLAAWSARTKIPLIPGQVQA |
| | | TNGLFDRIGSIYDGVEKKVTNRNANKKLEYDEAIKEGRNPAV |
| | | PEYETAYNIDGTLINKPGYNPNLYITQSRTPRLITEADRPLVEK |
| | | ILWQMVEKKTQSRNQARRARLEKAAHLQGLPVPKFVPEKV |
| | | DRSQKIEIRIIDPLDKIEPYMPQDRMAIKASQDGHVPYWQRPF |
| | | LSKRRNRRVRAGWGKQVSSIQAWLTGALLVIVRLGNEAFLA |
| | | DIRGALRNAQWRKLLKPDATYQSLFNLFTGDPVVNTRTNHL |
| | | TMAYREGVVDIVKSRSFKGRQTREHLLTLLGQGKTVAGVSF |
| | | DLGQKHAAGLLAAHFGLGEDGNPVFTPIQACFLPQRYLDSLT |
| | | NYRNRYDALTLDMRRQSLLALTPAQQQEFADAQRDPGGQA |
| | | KRACCLKLNLNPDEIRWDLVSGISTMISDLYIERGGDPRDVH |
| | | QQVETKPKGKRKSEIRILKIRDGKWAYDFRPKIADETRKAQR |
| | | EQLWKLQKASSEFERLSRYKINIARAIANWALQWGRELSGC |
| | | DIVIPVLEDLNVGSKFFDGKGKWLLGWDNRFTPKKENRWFI |
| | | KVLHKAVAELAPHRGVPVYEVMPHRTSMTCPACHYCHPTN |
| | | REGDRFECQSCHVVKNTDRDVAPYNILRVAVEGKTLDRWQ |
| | | AEKKPQAEPDRPMILIDNQES |
| CasΦ.10 | 10 | MDMLDTETNYATETPSQQQDYSPKPPKKDRRAPKGFSKKAR |
| | | PEKKPPKPITLFTQKHFSGVRFLKRVIRDASKILKLSESRTITFL |
| | | EQAIERDGSAPPDVTPPVHNTIMAVTRPFEEWPEVILSKALQK |
| | | HCYALTKKIKIKTWPKKGPGKKCLAAWSARTKIPLIPGQVQA |
| | | TNGLFDRIGSIYDGVEKKVTNRNANKKLEYDEAIKEGRNPAV |
| | | PEYETAYNIDGTLINKPGYNPNLYITQSRTPRLITEADRPLVEK |
| | | ILWQMVEKKTQSRNQARRARLEKAAHLQGLPVPKFVPEKV |
| | | DRSQKIEIRIIDPLDKIEPYMPQDRMAIKASQDGHVPYWQRPF |
| | | LSKRRNRRVRAGWGKQVSSIQAWLTGALLVIVRLGNEAFLA |
| | | DIRGALRNAQWRKLLKPDATYQSLFNLFTGDPVVNTRTNHL |
| | | TMAYREGVVNIVKSRSFKGRQTREHLLTLLGQGKTVAGVSF |
| | | DLGQKHAAGLLAAHFGLGEDGNPVFTPIQACFLPQRYLDSLT |
| | | NYRNRYDALTLDMRRQSLLALTPAQQQEFADAQRDPGGQA |
| | | KRACCLKLNLNPDEIRWDLVSGISTMISDLYIERGGDPRDVH |
| | | QQVETKPKGKRKSEIRILKIRDGKWAYDFRPKIADETRKAQR |
| | | EQLWKLQKASSEFERLSRYKINIARAIANWALQWGRELSGC |
| | | DIVIPVLEDLNVGSKFFDGKGKWLLGWDNRFTPKKENRWFI |
| | | KVLHKAVAELAPHRGVPVYEVMPHRTSMTCPACHYCHPTN |
| | | REGDRFECQSCHVVKNTDRDVAPYNILRVAVEGKTLDRWQ |
| | | AEKKPQAEPDRPMILIDNQES |
| CasΦ.11 | 11 | MSNKTTPPSPLSLLLRAHFPGLKFESQDYKIAGKKLRDGGPE |
| | | AVISYLTGKGQAKLKDVKPPAKAFVIAQSRPFIEWDLVRVSR |
| | | QIQEKIFGIPATKGRPKQDGLSETAFNEAVASLEVDGKSKLNE |
| | | ETRAAFYEVLGLDAPSLHAQAQNALIKSAISIREGVLKKVEN |
| | | RNEKNLSKTKRRKEAGEEATFVEEKAHDERGYLIHPPGVNQ |
| | | TIPGYQAVVIKSCPSDFIGLPSGCLAKESAEALTDYLPHDRMT |
| | | IPKGQPGYVPEWQHPLLNRRKNRRRRDWYSASLNKPKATCS |
| | | KRSGTPNRKNSRTDQIQSGRFKGAIPVLMRFQDEWVIIDIRGL |
| | | LRNARYRKLLKEKSTIPDLLSLFTGDPSIDMRQGVCTFIYKAG |
| | | QACSAKMVKTKNAPEILSELTKSGPVVLVSIDLGQTNPIAAK |
| | | VSRVTQLSDGQLSHETLLRELLSNDSSDGKEIARYRVASDRL |
| | | RDKLANLAVERLSPEHKSEILRAKNDTPALCKARVCAALGL |
| | | NPEMIAWDKMTPYTEFLATAYLEKGGDRKVATLKPKNRPE |
| | | MLRRDIKFKGTEGVRIEVSPEAAEAYREAQWDLQRTSPEYLR |
| | | LSTWKQELTKRILNQLRHKAAKSSQCEVVVMAFEDLNIKMM |
| | | HGNGKWADGGWDAFFIKKRENRWFMQAFHKSLTELGAHK |
| | | GVPTIEVTPHRTSITCTKCGHCDKANRDGERFACQKCGFVAH |
| | | ADLEIATDNIERVALTGKPMPKPESERSGDAKKSVGARKAAF |
| | | KPEEDAEAAE |
| CasΦ.12 | 12 | MIKPTVSQFLTPGFKLIRNHSRTAGLKLKNEGEEACKKFVRE |
| | | NEIPKDECPNFQGGPAIANIIAKSREFTEWEIYQSSLAIQEVIFT |
| | | LPKDKLPEPILKEEWRAQWLSEHGLDTVPYKEAAGLNLIIKN |
| | | AVNTYKGVQVKVDNKNKNNLAKINRKNEIAKLNGEQEISFE |
| | | EIKAFDDKGYLLQKPSPNKSIYCYQSVSPKPFITSKYHNVNLP |
| | | EEYIGYYRKSNEPIVSPYQFDRLRIPIGEPGYVPKWQYTFLSK |
| | | KENKRRKLSKRIKNVSPILGIICIKKDWCVFDMRGLLRTNHW |
| | | KKYHKPTDSINDLFDYFTGDPVIDTKANVVRFRYKMENGIV |
| | | NYKPVREKKGKELLENICDQNGSCKLATVDVGQNNPVAIGL |
| | | FELKKVNGELTKTLISRHPTPIDFCNKITAYRERYDKLESSIKL |
| | | DAIKQLTSEQKIEVDNYNNNFTPQNTKQIVCSKLNINPNDLP |
| | | WDKMISGTHFISEKAQVSNKSEIYFTSTDKGKTKDVMKSDY |
| | | KWFQDYKPKLSKEVRDALSDIEWRLRRESLEFNKLSKSREQ |
| | | DARQLANWISSMCDVIGIENLVKKNNFFGGSGKREPGWDNF |
| | | YKPKKENRWWINAIHKALTELSQNKGKRVILLPAMRTSITCP |
| | | KCKYCDSKNRNGEKFNCLKCGIELNADIDVATENLATVAITA |
| | | QSMPKPTCERSGDAKKPVRARKAKAPEFHDKLAPSYTVVLR |
| | | EAV |
| CasΦ.13 | 13 | MRQPAEKTAFQVFRQEVIGTQKLSGGDAKTAGRLYKQGKM |
| | | EAAREWLLKGARDDVPPNFQPPAKCLVVAVSHPFEEWDISK |
| | | TNHDVQAYIYAQPLQAEGHLNGLSEKWEDTSADQHKLWFE |
| | | KTGVPDRGLPVQAINKIAKAAVNRAFGVVRKVENRNEKRRS |
| | | RDNRIAEHNRENGLTEVVREAPEVATNADGFLLHPPGIDPSIL |
| | | SYASVSPVPYNSSKHSFVRLPEEYQAYNVEPDAPIPQFVVED |
| | | RFAIPPGQPGYVPEWQRLKCSTNKHRRMRQWSNQDYKPKA |
| | | GRRAKPLEFQAHLTRERAKGALLVVMRIKEDWVVFDVRGL |
| | | LRNVEWRKVLSEEAREKLTLKGLLDLFTGDPVIDTKRGIVTF |
| | | LYKAEITKILSKRTVKTKNARDLLLRLTEPGEDGLRREVGLV |
| | | AVDLGQTHPIAAAIYRIGRTSAGALESTVLHRQGLREDQKEK |
| | | LKEYRKRHTALDSRLRKEAFETLSVEQQKEIVTVSGSGAQIT |
| | | KDKVCNYLGVDPSTLPWEKMGSYTHFISDDFLRRGGDPNIV |
| | | HFDRQPKKGKVSKKSQRIKRSDSQWVGRMRPRLSQETAKAR |
| | | MEADWAAQNENEEYKRLARSKQELARWCVNTLLQNTRCIT |
| | | QCDEIVVVIEDLNVKSLHGKGAREPGWDNFFTPKTENRWFIQ |
| | | ILHKTFSELPKHRGEHVIEGCPLRTSITCPACSYCDKNSRNGE |
| | | KFVCVACGATFHADFEVATYNLVRLATTGMPMPKSLERQG |
| | | GGEKAGGARKARKKAKQVEKIVVQANANVTMNGASLHSP |
| CasΦ.14 | 14 | MSSLPTPLELLKQKHADLFKGLQFSSKDNKMAGKVLKKDGE |
| | | EAALAFLSERGVSRGELPNFRPPAKTLVVAQSRPFEEFPIYRV |
| | | SEAIQLYVYSLSVKELETVPSGSSTKKEHQRFFQDSSVPDFGY |
| | | TSVQGLNKIFGLARGIYLGVITRGENQLQKAKSKHEALNKKR |
| | | RASGEAETEFDPTPYEYMTPERKLAKPPGVNHSIMCYVDISV |
| | | DEFDFRNPDGIVLPSEYAGYCREINTAIEKGTVDRLGHLKGG |
| | | PGYIPGHQRKESTTEGPKINFRKGRIRRSYTALYAKRDSRRVR |
| | | QGKLALPSYRHHMMRLNSNAESAILAVIFFGKDWVVFDLRG |
| | | LLRNVRWRNLFVDGSTPSTLLGMFGDPVIDPKRGVVAFCYK |
| | | EQIVPVVSKSITKMVKAPELLNKLYLKSEDPLVLVAIDLGQT |
| | | NPVGVGVYRVMNASLDYEVVTRFALESELLREIESYRQRTN |
| | | AFEAQIRAETFDAMTSEEQEEITRVRAFSASKAKENVCHRFG |
| | | MPVDAVDWATMGSNTIHIAKWVMRHGDPSLVEVLEYRKDN |
| | | EIKLDKNGVPKKVKLTDKRIANLTSIRLRFSQETSKHYNDTM |
| | | WELRRKHPVYQKLSKSKADFSRRVVNSIIRRVNHLVPRARIV |
| | | FILEDLKNLGKVFHGSGKRELGWDSYFEPKSENRWFIQVLHK |
| | | AFSETGKHKGYYIIECWPNWTSCTCPKCSCCDSENRHGEVFR |
| | | CLACGYTCNTDFGTAPDNLVKIATTGKGLPGPKKRCKGSSK |
| | | GKNPKIARSSETGVSVTESGAPKVKKSSPTQTSQSSSQSAP |
| CasΦ.15 | 15 | MIKPTVSQFLTPGFKLIRNHSRTAGLKLKNEGEEACKKFVRE |
| | | NEIPKDECPNFQGGPAIANIIAKSREFTEWEIYQSSLAIQEVIFT |
| | | LPKDKLPEPILKEEWRAQWLSEHGLDTVPYKEAAGLNLIIKN |
| | | AVNTYKGVQVKVDNKNKNNLAKINRKNEIAKLNGEQEISFE |
| | | EIKAFDDKGYLLQKPSPNKSIYCYQSVSPKPFITSKYHNVNLP |
| | | EEYIGYYRKSNEPIVSPYQFDRLRIPIGEPGYVPKWQYTFLSK |
| | | KENKRRKLSKRIKNVSPILGIICIKKDWCVFDMRGLLRTNHW |
| | | KKYHKPTDSINDLFDYFTGDPVIDTKANVVRFRYKMENGIV |
| | | NYKPVREKKGKELLENICDQNGSCKLATVDVGQNNPVAIGL |
| | | FELKKVNGELTKTLISRHPTPIDFCNKITAYRERYDKLESSIKL |
| | | DAIKQLTSEQKIEVDNYNNNFTPQNTKQIVCSKLNINPNDLP |
| | | WDKMISGTHFISEKAQVSNKSEIYFTSTDKGKTKDVMKSDY |
| | | KWFQDYKPKLSKEVRDALSDIEWRLRRESLEFNKLSKSREQ |
| | | DARQLANWISSMCDVIGIENLVKKNNFFGGSGKREPGWDNF |
| | | YKPKKENRWWINAIHKALTELSQNKGKRVILLPAMRTSITCP |
| | | KCKYCDSKNRNGEKFNCLKCGIELNADIDVATENLATVAITA |
| | | QSMPKPTCERSGDAKKPVRARKAKAPEFHDKLAPSYTVVLR |
| | | EAV |
| CasΦ.16 | 16 | MSNKTTPPSPLSLLLRAHFPGLKFESQDYKIAGKKLRDGGPE |
| | | AVISYLTGKGQAKLKDVKPPAKAFVIAQSRPFIEWDLVRVSR |
| | | QIQEKIFGIPATKGRPKQDGLSETAFNEAVASLEVDGKSKLNE |
| | | ETRAAFYEVLGLDAPSLHAQAQNALIKSAISIREGVLKKVEN |
| | | RNEKNLSKTKRRKEAGEEATFVEEKAHDERGYLIHPPGVNQ |
| | | TIPGYQAVVIKSCPSDFIGLPSGCLAKESAEALTDYLPHDRMT |
| | | IPKGQPGYVPEWQHPLLNRRKNRRRRDWYSASLNKPKATCS |
| | | KRSGTPNRKNSRTDQIQSGRFKGAIPVLMRFQDEWVIIDIRGL |
| | | LRNARYRKLLKEKSTIPDLLSLFTGDPSIDMRQGVCTFIYKAG |
| | | QACSAKMVKTKNAPEILSELTKSGPVVLVSIDLGQTNPIAAK |
| | | VSRVTQLSDGQLSHETLLRELLSNDSSDGKEIARYRVASDRL |
| | | RDKLANLAVERLSPEHKSEILRAKNDTPALCKARVCAALGL |
| | | NPEMIAWDKMTPYTEFLATAYLEKGGDRKVATLKPKNRPE |
| | | MLRRDIKFKGTEGVRIEVSPEAAEAYREAQWDLQRTSPEYLR |
| | | LSTWKQELTKRILNQLRHKAAKSSQCEVVVMAFEDLNIKMM |
| | | HGNGKWADGGWDAFFIKKRENRWFMQAFHKSLTELGAHK |
| | | GVPTIEVTPHRTSITCTKCGHCDKANRDGERFACQKCGFVAH |
| | | ADLEIATDNIERVALTGKPMPKPESERSGDAKKSVGARKAAF |
| | | KPEEDAEAAE |
| CasΦ.17 | 17 | MYSLEMADLKSEPSLLAKLLRDRFPGKYWLPKYWKLAEKK |
| | | RLTGGEEAACEYMADKQLDSPPPNFRPPARCVILAKSRPFED |
| | | WPVHRVASKAQSFVIGLSEQGFAALRAAPPSTADARRDWLR |
| | | SHGASEDDLMALEAQLLETIMGNAISLHGGVLKKIDNANVK |
| | | AAKRLSGRNEARLNKGLQELPPEQEGSAYGADGLLVNPPGL |
| | | NLNIYCRKSCCPKPVKNTARFVGHYPGYLRDSDSILISGTMD |
| | | RLTIIEGMPGHIPAWQREQGLVKPGGRRRRLSGSESNMRQKV |
| | | DPSTGPRRSTRSGTVNRSNQRTGRNGDPLLVEIRMKEDWVL |
| | | LDARGLLRNLRWRESKRGLSCDHEDLSLSGLLALFSGDPVID |
| | | PVRNEVVFLYGEGIIPVRSTKPVGTRQSKKLLERQASMGPLT |
| | | LISCDLGQTNLIAGRASAISLTHGSLGVRSSVRIELDPEIIKSFE |
| | | RLRKDADRLETEILTAAKETLSDEQRGEVNSHEKDSPQTAKA |
| | | SLCRELGLHPPSLPWGQMGPSTTFIADMLISHGRDDDAFLSH |
| | | GEFPTLEKRKKFDKRFCLESRPLLSSETRKALNESLWEVKRTS |
| | | SEYARLSQRKKEMARRAVNFVVEISRRKTGLSNVIVNIEDLN |
| | | VRIFHGGGKQAPGWDGFFRPKSENRWFIQAIHKAFSDLAAH |
| | | HGIPVIESDPQRTSMTCPECGHCDSKNRNGVRFLCKGCGASM |
| | | DADFDAACRNLERVALTGKPMPKPSTSCERLLSATTGKVCS |
| | | DHSLSHDAIEKAS |
| CasΦ.18 | 18 | MEKEITELTKIRREFPNKKFSSTDMKKAGKLLKAEGPDAVRD |
| | | FLNSCQEIIGDFKPPVKTNIVSISRPFEEWPVSMVGRAIQEYYF |
| | | SLTKEELESVHPGTSSEDHKSFFNITGLSNYNYTSVQGLNLIF |
| | | KNAKAIYDGTLVKANNKNKKLEKKFNEINHKRSLEGLPIITP |
| | | DFEEPFDENGHLNNPPGINRNIYGYQGCAAKVFVPSKHKMV |
| | | SLPKEYEGYNRDPNLSLAGFRNRLEIPEGEPGHVPWFQRMDI |
| | | PEGQIGHVNKIQRFNFVHGKNSGKVKFSDKTGRVKRYHHSK |
| | | YKDATKPYKFLEESKKVSALDSILAIITIGDDWVVFDIRGLYR |
| | | NVFYRELAQKGLTAVQLLDLFTGDPVIDPKKGVVTFSYKEG |
| | | VVPVFSQKIVPRFKSRDTLEKLTSQGPVALLSVDLGQNEPVA |
| | | ARVCSLKNINDKITLDNSCRISFLDDYKKQIKDYRDSLDELEI |
| | | KIRLEAINSLETNQQVEIRDLDVFSADRAKANTVDMFDIDPN |
| | | LISWDSMSDARVSTQISDLYLKNGGDESRVYFEINNKRIKRS |
| | | DYNISQLVRPKLSDSTRKNLNDSIWKLKRTSEEYLKLSKRKL |
| | | ELSRAVVNYTIRQSKLLSGINDIVIILEDLDVKKKFNGRGIRDI |
| | | GWDNFFSSRKENRWFIPAFHKTFSELSSNRGLCVIEVNPAWT |
| | | SATCPDCGFCSKENRDGINFTCRKCGVSYHADIDVATLNIAR |
| | | VAVLGKPMSGPADRERLGDTKKPRVARSRKTMKRKDISNST |
| | | VEAMVTA |
| CasΦ.19 | 19 | MLVRTSTLVQDNKNSRSASRAFLKKPKMPKNKHIKEPTELA |
| | | KLIRELFPGQRFTRAINTQAGKILKHKGRDEVVEFLKNKGIDK |
| | | EQFMDFRPPTKARIVATSGAIEEFSYLRVSMAIQECCFGKYKF |
| | | PKEKVNGKLVLETVGLTKEELDDFLPKKYYENKKSRDRFFL |
| | | KTGICDYGYTYAQGLNEIFRNTRAIYEGVFTKVNNRNEKRRE |
| | | KKDKYNEERRSKGLSEEPYDEDESATDESGHLINPPGVNLNI |
| | | WTCEGFCKGPYVTKLSGTPGYEVILPKVFDGYNRDPNEIISC |
| | | GITDRFAIPEGEPGHIPWHQRLEIPEGQPGYVPGHQRFADTGQ |
| | | NNSGKANPNKKGRMRKYYGHGTKYTQPGEYQEVFRKGHRE |
| | | GNKRRYWEEDFRSEAHDCILYVIHIGDDWVVCDLRGPLRDA |
| | | YRRGLVPKEGITTQELCNLFSGDPVIDPKHGVVTFCYKNGLV |
| | | RAQKTISAGKKSRELLGALTSQGPIALIGVDLGQTEPVGARAF |
| | | IVNQARGSLSLPTLKGSFLLTAENSSSWNVFKGEIKAYREAID |
| | | DLAIRLKKEAVATLSVEQQTEIESYEAFSAEDAKQLACEKFG |
| | | VDSSFILWEDMTPYHTGPATYYFAKQFLKKNGGNKSLIEYIP |
| | | YQKKKSKKTPKAVLRSDYNIACCVRPKLLPETRKALNEAIRI |
| | | VQKNSDEYQRLSKRKLEFCRRVVNYLVRKAKKLTGLERVII |
| | | AIEDLKSLEKFFTGSGKRDNGWSNFFRPKKENRWFIPAFHKA |
| | | FSELAPNRGFYVIECNPARTSITDPDCGYCDGDNRDGIKFECK |
| | | KCGAKHHTDLDVAPLNIAIVAVTGRPMPKTVSNKSKRERSG |
| | | GEKSVGASRKRNHRKSKANQEMLDATSSAAE |
| CasΦ.20 | 20 | MPKIKKPTEISLLRKEVFPDLHFAKDRMRAASLVLKNEGREA |
| | | AIEYLRVNHEDKPPNFMPPAKTPYVALSRPLEQWPIAQASIAI |
| | | QKYIFGLTKDEFSATKKLLYGDKSTPNTESRKRWFEVTGVPN |
| | | FGYMSAQGLNAIFSGALARYEGVVQKVENRNKKRFEKLSEK |
| | | NQLLIEEGQPVKDYVPDTAYHTPETLQKLAENNHVRVEDLG |
| | | DMIDRLVHPPGIHRSIYGYQQVPPFAYDPDNPKGIILPKAYAG |
| | | YTRKPHDIIEAMPNRLNIPEGQAGYIPEHQRDKLKKGGRVKR |
| | | LRTTRVRVDATETVRAKAEALNAEKARLRGKEAILAVFQIEE |
| | | DWALIDMRGLLRNVYMRKLIAAGELTPTTLLGYFTETLTLDP |
| | | RRTEATFCYHLRSEGALHAEYVRHGKNTRELLLDLTKDNEKI |
| | | ALVTIDLGQRNPLAAAIFRVGRDASGDLTENSLEPVSRMLLP |
| | | QAYLDQIKAYRDAYDSFRQNIWDTALASLTPEQQRQILAYE |
| | | AYTPDDSKENVLRLLLGGNVMPDDLPWEDMTKNTHYISDR |
| | | YLADGGDPSKVWFVPGPRKRKKNAPPLKKPPKPRELVKRSD |
| | | HNISHLSEFRPQLLKETRDAFEKAKIDTERGHVGYQKLSTRK |
| | | DQLCKEILNWLEAEAVRLTRCKTMVLGLEDLNGPFFNQGKG |
| | | KVRGWVSFFRQKQENRWIVNGFRKNALARAHDKGKYILEL |
| | | WPSWTSQTCPKCKHVHADNRHGDDFVCLQCGARLHADAEV |
| | | ATWNLAVVAIQGHSLPGPVREKSNDRKKSGSARKSKKANES |
| | | GKVVGAWAAQATPKRATSKKETGTARNPVYNPLETQASCP |
| | | AP |
| CasΦ.21 | 21 | MTPSPQIARLVETPLAAALKAHHPGKKFRSDYLKKAGKILKD |
| | | QGVEAAMAHLDGKDQAEPPNFKPPAKCRIVARSREFSEWPI |
| | | VKASVEIQKYIYGLTLEERKACDPGKSSASHKAWFAKTGVN |
| | | TFGYSSVQGFNLIFGHTLGRYDGVLVKTENLNKKRAEKNER |
| | | FRAKALAEGRAEPVCPPLVTATNDTGQDVTLEDGRVVRPGQ |
| | | LLQPPGINPNIYAYQQVSPKAYVPGIIELPEEFQGYSRDPNAVI |
| | | LPLVPRDRLSIPKGQPGYVPEPHREGLTGRKDRRMRRYYETE |
| | | RGTKLKRPPLTAKGRADKANEALLVVVRIDSDWVVMDVRG |
| | | LLRNARWRRLVSKEGITLNGLLDLFTGDPVLNPKDCSVSRDT |
| | | GDPVNDPRHGVVTFCYKLGVVDVCSKDRPIKGFRTKEVLER |
| | | LTSSGTVGMVSIDLGQTNPVAAAVSRVTKGLQAETLETFTLP |
| | | DDLLGKVRAYRAKTDRMEEGFRRNALRKLTAEQQAEITRYN |
| | | DATEQQAKALVCSTYGIGPEEVPWERMTSNTTYISDHILDHG |
| | | GDPDTVFFMATKRGQNKPTLHKRKDKAWGQKFRPAISVETR |
| | | LARQAAEWELRRASLEFQKLSVWKTELCRQAVNYVMERTK |
| | | KRTQCDVIIPVIEDLPVPLFHGSGKRDPGWANFFVHKRENRW |
| | | FIDGLHKAFSELGKHRGIYVFEVCPQRTSITCPKCGHCDPDNR |
| | | DGEKFVCLSCQATLNADLDVATTNLVRVALTGKVMPRSERS |
| | | GDAQTPGPARKARTGKIKGSKPTSAPQGATQTDAKAHLSQT |
| | | GV |
| CasΦ.22 | 22 | MTPSPQIARLVETPLAAALKAHHPGKKFRSDYLKKAGKILKD |
| | | QGVEAAMAHLDGKDQAEPPNFKPPAKCRIVARSREFSEWPI |
| | | VKASVEIQKYIYGLTLEERKACDPGKSSASHKAWFAKTGVN |
| | | TFGYSSVQGFNLIFGHTLGRYDGVLVKTENLNKKRAEKNER |
| | | FRAKALAEGRAEPVCPPLVTATNDTGQDVTLEDGRVVRPGQ |
| | | LLQPPGINPNIYAYQQVSPKAYVPGIIELPEEFQGYSRDPNAVI |
| | | LPLVPRDRLSIPKGQPGYVPEPHREGLTGRKDRRMRRYYETE |
| | | RGTKLKRPPLTAKGRADKANEALLVVVRIDSDWVVMDVRG |
| | | LLRNARWRRLVSKEGITLNGLLDLFTGDPVLNPKDCSVSRDT |
| | | GDPVNDPRHGVVTFCYKLGVVDVCSKDRPIKGFRTKEVLER |
| | | LTSSGTVGMVSIDLGQTNPVAAAVSRVTKGLQAETLETFTLP |
| | | DDLLGKVRAYRAKTDRMEEGFRRNALRKLTAEQQAEITRYN |
| | | DATEQQAKALVCSTYGIGPEEVPWERMTSNTTYISDHILDHG |
| | | GDPDTVFFMATKRGQNKPTLHKRKDKAWGQKFRPAISVETR |
| | | LARQAAEWELRRASLEFQKLSVWKTELCRQAVNYVMERTK |
| | | KRTQCDVIIPVIEDLPVPLFHGSGKRDPGWANFFVHKRENRW |
| | | FIDGLHKAFSELGKHRGIYVFEVCPQRTSITCPKCGHCDPDNR |
| | | DGEKFVCLSCQATLHADLDVATTNLVRVALTGKVMPRSERS |
| | | GDAQTPGPARKARTGKIKGSKPTSAPQGATQTDAKAHLSQT |
| | | GV |
| CasΦ.23 | 23 | MKTEKPKTALTLLREEVFPGKKYRLDVLKEAGKKLSTKGRE |
| | | ATIEFLTGKDEERPQNFQPPAKTSIVAQSRPFDQWPIVQVSLA |
| | | VQKYIYGLTQSEFEANKKALYGETGKAISTESRRAWFEATGV |
| | | DNFGFTAAQGINPIFSQAVARYEGVIKKVENRNEKKLKKLTK |
| | | KNLLRLESGEEIEDFEPEATFNEEGRLLQPPGANPNIYCYQQIS |
| | | PRIYDPSDPKGVILPQIYAGYDRKPEDIISAGVPNRLAIPEGQP |
| | | GYIPEHQRAGLKTQGRIRCRASVEAKARAAILAVVHLGEDW |
| | | VVLDLRGLLRNVYWRKLASPGTLTLKGLLDFFTGGPVLDAR |
| | | RGIATFSYTLKSAAAVHAENTYKGKGTREVLLKLTENNSVA |
| | | LVTVDLGQRNPLAAMIARVSRTSQGDLTYPESVEPLTRLFLP |
| | | DPFLEEVRKYRSSYDALRLSIREAAIASLTPEQQAEIRYIEKFS |
| | | AGDAKKNVAEVFGIDPTQLPWDAMTPRTTYISDLFLRMGGD |
| | | RSRVFFEVPPKKAKKAPKKPPKKPAGPRIVKRTDGMIARLREI |
| | | RPRLSAETNKAFQEARWEGERSNVAFQKLSVRRKQFARTVV |
| | | NHLVQTAQKMSRCDTVVLGIEDLNVPFFHGRGKYQPGWEG |
| | | FFRQKKENRWLINDMHKALSERGPHRGGYVLELTPFWTSLR |
| | | CPKCGHTDSANRDGDDFVCVKCGAKLHSDLEVATANLALV |
| | | AITGQSIPRPPREQSSGKKSTGTARMKKTSGETQGKGSKACV |
| | | SEALNKIEQGTARDPVYNPLNSQVSCPAP |
| CasΦ.24 | 24 | VYNPDMKKPNNIRRIREEHFEGLCFGKDVLTKAGKIYEKDGE |
| | | EAAIDFLMGKDEEDPPNFKPPAKTTIVAQSRPFDQWPIYQVS |
| | | QAVQERVFAYTEEEFNASKEALFSGDISSKSRDFWFKTNNIS |
| | | DQGIGAQGLNTILSHAFSRYSGVIKKVENRNKKRLKKLSKKN |
| | | QLKIEEGLEILEFKPDSAFNENGLLAQPPGINPNIYGYQAVTPF |
| | | VFDPDNPGDVILPKQYEGYSRKPDDIIEKGPSRLDIPKGQPGY |
| | | VPEHQRKNLKKKGRVRLYRRTPPKTKALASILAVLQIGKDW |
| | | VLFDMRGLLRSVYMREAATPGQISAKDLLDTFTGCPVLNTR |
| | | TGEFTFCYKLRSEGALHARKIYTKGETRTLLTSLTSENNTIAL |
| | | VTVDLGQRNPAAIMISRLSRKEELSEKDIQPVSRRLLPDRYLN |
| | | ELKRYRDAYDAFRQEVRDEAFTSLCPEHQEQVQQYEALTPE |
| | | KAKNLVLKHFFGTHDPDLPWDDMTSNTHYIANLYLERGGDP |
| | | SKVFFTRPLKKDSKSKKPRKPTKRTDASISRLPEIRPKMPEDA |
| | | RKAFEKAKWEIYTGHEKFPKLAKRVNQLCREIANWIEKEAK |
| | | RLTLCDTVVVGIEDLSLPPKRGKGKFQETWQGFFRQKFENR |
| | | WVIDTLKKAIQNRAHDKGKYVLGLAPYWTSQRCPACGFIHK |
| | | SNRNGDHFKCLKCEALFHADSEVATWNLALVAVLGKGITNP |
| | | DSKKPSGQKKTGTTRKKQIKGKNKGKETVNVPPTTQEVEDII |
| | | AFFEKDDETVRNPVYKPTGT |
| CasΦ.25 | 25 | MKKPNNIRRIREEHFEGLCFGKDVLTKAGKIYEKDGEEAAID |
| | | FLMGKDEEDPPNFKPPAKTTIVAQSRPFDQWPIYQVSQAVQE |
| | | RVFAYTEEEFNASKEALFSGDISSKSRDFWFKTNNISDQGIGA |
| | | QGLNTILSHAFSRYSGVIKKVENRNKKRLKKLSKKNQLKIEE |
| | | GLEILEFKPDSAFNENGLLAQPPGINPNIYGYQAVTPFVFDPD |
| | | NPGDVILPKQYEGYSRKPDDIIEKGPSRLDIPKGQPGYVPEHQ |
| | | RKNLKKKGRVRLYRRTPPKTKALASILAVLQIGKDWVLFDM |
| | | RGLLRSVYMREAATPGQISAKDLLDTFTGCPVLNTRTGEFTF |
| | | CYKLRSEGALHARKIYTKGETRTLLTSLTSENNTIALVTVDL |
| | | GQRNPAAIMISRLSRKEELSEKDIQPVSRRLLPDRYLNELKRY |
| | | RDAYDAFRQEVRDEAFTSLCPEHQEQVQQYEALTPEKAKNL |
| | | VLKHFFGTHDPDLPWDDMTSNTHYIANLYLERGGDPSKVFF |
| | | TRPLKKDSKSKKPRKPTKRTDASISRLPEIRPKMPEDARKAFE |
| | | KAKWEIYTGHEKFPKLAKRVNQLCREIANWIEKEAKRLTLC |
| | | DTVVVGIEDLSLPPKRGKGKFQETWQGFFRQKFENRWVIDT |
| | | LKKAIQNRAHDKGKYVLGLAPYWTSQRCPACGFIHKSNRNG |
| | | DHFKCLKCEALFHADSEVATWNLALVAVLGKGITNPDSKKP |
| | | SGQKKTGTTRKKQIKGKNKGKETVNVPPTTQEVEDIIAFFEK |
| | | DDETVRNPVYKPTGT |
| CasΦ.26 | 26 | VIKTHFPAGRFRKDHQKTAGKKLKHEGEEACVEYLRNKVSD |
| | | YPPNFKPPAKGTIVAQSRPFSEWPIVRASEAIQKYVYGLTVAE |
| | | LDVFSPGTSKPSHAEWFAKTGVENYGYRQVQGLNTIFQNTV |
| | | NRFKGVLKKVENRNKKSLKRQEGANRRRVEEGLPEVPVTVE |
| | | SATDDEGRLLQPPGVNPSIYGYQGVAPRVCTDLQGFSGMSV |
| | | DFAGYRRDPDAVLVESLPEGRLSIPKGERGYVPEWQRDPERN |
| | | KFPLREGSRRQRKWYSNACHKPKPGRTSKYDPEALKKASAK |
| | | DALLVSISIGEDWAIIDVRGLLRDARRRGFTPEEGLSLNSLLG |
| | | LFTEYPVFDVQRGLITFTYKLGQVDVHSRKTVPTFRSRALLES |
| | | LVAKEEIALVSVDLGQTNPASMKVSRVRAQEGALVAEPVHR |
| | | MFLSDVLLGELSSYRKRMDAFEDAIRAQAFETMTPEQQAEIT |
| | | RVCDVSVEVARRRVCEKYSISPQDVPWGEMTGHSTFIVDAV |
| | | LRKGGDESLVYFKNKEGETLKFRDLRISRMEGVRPRLTKDTR |
| | | DALNKAVLDLKRAHPTFAKLAKQKLELARRCVNFIEREAKR |
| | | YTQCERVVFVIEDLNVGFFHGKGKRDRGWDAFFTAKKENR |
| | | WVIQALHKAFSDLGLHRGSYVIEVTPQRTSMTCPRCGHCDK |
| | | GNRNGEKFVCLQCGATLHADLEVATDNIERVALTGKAMPKP |
| | | PVRERSGDVQKAGTARKARKPLKPKQKTEPSVQEGSSDDGV |
| | | DKSPGDASRNPVYNPSDTLSI |
| CasΦ.27 | 27 | MAKAKTLAALLRELLPGQHLAPHHRWVANKLLMTSGDAAA |
| | | FVIGKSVSDPVRGSFRKDVITKAGRIFKKDGPDAAAAFLDGK |
| | | WEDRPPNFQPPAKAAIVAISRSFDEWPIVKVSCAIQQYLYALP |
| | | VQEFESSVPEARAQAHAAWFQDTGVDDCNFKSTQGLNAIFN |
| | | HGKRTYEGVLKKAQNRNDKKNLRLERINAKRAEAGQAPLV |
| | | AGPDESPTDDAGCLLHPPGINANIYCYQQVSPRPYEQSCGIQL |
| | | PPEYAGYNRLSNVAIPPMPNRLDIPQGQPGYVPEHHRHGIKK |
| | | FGRVRKRYGVVPGRNRDADGKRTRQVLTEAGAAAKARDSV |
| | | LAVIRIGDDWTVVDLRGLLRNAQWRKLVPDGGITVQGLLDL |
| | | FTGDPVIDPRRGVVTFIYKADSVGIHSEKVCRGKQSKNLLER |
| | | LCAMPEKSSTRLDCARQAVALVSVDLGQRNPVAARFSRVSL |
| | | AEGQLQAQLVSAQFLDDAMVAMIRSYREEYDRFESLVREQA |
| | | KAALSPEQLSEIVRHEADSAESVKSCVCAKFGIDPAGLSWDK |
| | | MTSGTWRIADHVQAAGGDVEWFFFKTCGKGKEIKTVRRSDF |
| | | NVAKQFRLRLSPETRKDWNDAIWELKRGNPAYVSFSKRKSE |
| | | FARRVVNDLVHRARRAVRCDEVVFAIEDLNISFFHGKGQRQ |
| | | MGWDAFFEVKQENRWFIQALHKAFVERATHKGGYVLEVAP |
| | | ARTSTTCPECRHCDPESRRGEQFCCIKCRHTCHADLEVATFNI |
| | | EQVALTGVSLPKRLSSTLL |
| CasΦ.28 | 28 | MSKEKTPPSAYAILKAKHFPDLDFEKKHKMMAGRMFKNGA |
| | | SEQEVVQYLQGKGSESLMDVKPPAKSPILAQSRPFDEWEMV |
| | | RTSRLIQETIFGIPKRGSIPKRDGLSETQFNELVASLEVGGKPM |
| | | LNKQTRAIFYGLLGIKPPTFHAMAQNILIDLAINIRKGVLKKV |
| | | DNLNEKNRKKVKRIRDAGEQDVMVPAEVTAHDDRGYLNHP |
| | | PGVNPTIPGYQGVVIPFPEGFEGLPSGMTPVDWSHVLVDYLP |
| | | HDRLSIPKGSPGYIPEWQRPLLNRHKGRRHRSWYANSLNKPR |
| | | KSRTEEAKDRQNAGKRTALIEAERLKGVLPVLMRFKEDWLII |
| | | DARGLLRNARYRGVLPEGSTLGNLIDLFSDSPRVDTRRGICTF |
| | | LYRKGRAYSTKPVKRKESKETLLKLTEKSTIALVSIDLGQTNP |
| | | LTAKLSKVRQVDGCLVAEPVLRKLIDNASEDGKEIARYRVA |
| | | HDLLRARILEDAIDLLGIYKDEVVRARSDTPDLCKERVCRFL |
| | | GLDSQAIDWDRMTPYTDFIAQAFVAKGGDPKVVTIKPNGKP |
| | | KMFRKDRSIKNMKGIRLDISKEASSAYREAQWAIQRESPDFQ |
| | | RLAVWQSQLTKRIVNQLVAWAKKCTQCDTVVLAFEDLNIG |
| | | MMHGSGKWANGGWNALFLHKQENRWFMQAFHKALTELS |
| | | AHKGIPTIEVLPHRTSITCTQCGHCHPGNRDGERFKCLKCEFL |
| | | ANTDLEIATDNIERVALTGLPMPKGERSSAKRKPGGTRKTKK |
| | | SKHSGNSPLAAE |
| CasΦ.29 | 29 | MEKAGPTSPLSVLIHKNFEGCRFQIDHLKIAGRKLAREGEAA |
| | | AIEYLLDKKCEGLPPNFQPPAKGNVIAQSRPFTEWAPYRASV |
| | | AIQKYIYSLSVDERKVCDPGSSSDSHEKWFKQTGVQNYGYT |
| | | HVQGLNLIFKHALARYDGVLKKVDNRNEKNRKKAERVNSF |
| | | RREEGLPEEVFEEEKATDETGHLLQPPGVNHSIYCYQSVRPK |
| | | PFNPRKPGGISLPEAYSGYSLKPQDELPIGSLDRLSIPPGQPGY |
| | | VPEWQRSQLTTQKHRRKRSWYSAQKWKPRTGRTSTFDPDR |
| | | LNCARAQGAILAVVRIHEDWVVFDVRGLLRNALWRELAGK |
| | | GLTVRDLLDFFTGDPVVDTKRGVVTFTYKLGKVDVHSLRTV |
| | | RGKRSKKVLEDLTLSSDVGLVTIDLGQTNVLAADYSKVTRSE |
| | | NGELLAVPLSKSFLPKHLLHEVTAYRTSYDQMEEGFRRKALL |
| | | TLTEDQQVEVTLVRDFSVESSKTKLLQLGVDVTSLPWEKMS |
| | | SNTTYISDQLLQQGADPASLFFDGERDGKPCRHKKKDRTWA |
| | | YLVRPKVSPETRKALNEALWALKNTSPEFESLSKRKIQFSRR |
| | | CMNYLLNEAKRISGCGQVVFVIEDLNVRVHHGRGKRAIGWD |
| | | NFFKPKRENRWFMQALHKAASELAIHRGMHIIEACPARSSIT |
| | | CPKCGHCDPENRCSSDREKFLCVKCGAAFHADLEVATFNLR |
| | | KVALTGTALPKSIDHSRDGLIPKGARNRKLKEPQANDEKACA |
| CasΦ.30 | 30 | MKEQSPLSSVLKSNFPGKKFLSADIRVAGRKLAQLGEAAAVE |
| | | YLSPRQRDSVPNFRPPAFCTVVAKSRPFEEWPIYKASVLLQE |
| | | QIYGMTGQEFEERCGSIPTSLSGLRQWASSVGLGAAMEGLH |
| | | VQGMNLMVKNAINRYKGVLVKVENRNKKLVEANEAKNSS |
| | | REERGLPPLRPPELGSAFGPDGRLVNPPGIDKSIRLYQGVSPV |
| | | PVVKTTGRPTVHRLDIPAGEKGHVPLWQREAGLVKEGPRRR |
| | | RMWYSNSNLKRSRKDRSAEASEARKADSVVVRVSVKEDWV |
| | | DIDVRGLLRNVAWRGIERAGESTEDLLSLFSGDPVVDPSRDS |
| | | VVFLYKEGVVDVLSKKVVGAGKSRKQLEKMVSEGPVALVS |
| | | CDLGQTNYVAARVSVLDESLSPVRSFRVDPREFPSADGSQGV |
| | | VGSLDRIRADSDRLEAKLLSEAEASLPEPVRAEIEFLRSERPSA |
| | | VAGRLCLKLGIDPRSIPWEKMGSTTSFISEALSAKGSPLALHD |
| | | GAPIKDSRFAHAARGRLSPESRKALNEALWERKSSSREYGVI |
| | | SRRKSEASRRMANAVLSESRRLTGLAVVAVNLEDLNMVSKF |
| | | FHGRGKRAPGWAGFFTPKMENRWFIRSIHKAMCDLSKHRGI |
| | | TVIESRPERTSISCPECGHCDPENRSGERFSCKSCGVSLHADFE |
| | | VATRNLERVALTGKPMPRRENLHSPEGATASRKTRKKPREA |
| | | TASTFLDLRSVLSSAENEGSGPAARAG |
| CasΦ.31 | 31 | MLPPSNKIGKSMSLKEFINKRNFKSSIIKQAGKILKKEGEEAV |
| | | KKYLDDNYVEGYKKRDFPITAKCNIVASNRKIEDFDISKFSSF |
| | | IQNYVFNLNKDNFEEFSKIKYNRKSFDELYKKIANEIGLEKPN |
| | | YENIQGEIAVIRNAINIYNGVLKKVENRNKKIQEKNQSKDPPK |
| | | LLSAFDDNGFLAERPGINETIYGYQSVRLRHLDVEKDKDIIVQ |
| | | LPDIYQKYNKKSTDKISVKKRLNKYNVDEYGKLISKRRKERI |
| | | NKDDAILCVSNFGDDWIIFDARGLLRQTYRYKLKKKGLCIKD |
| | | LLNLFTGDPIINPTKTDLKEALSLSFKDGIINNRTLKVKNYKK |
| | | CPELISELIRDKGKVAMISIDLGQTNPISYRLSKFTANNVAYIE |
| | | NGVISEDDIVKMKKWREKSDKLENLIKEEAIASLSDDEQREV |
| | | RLYENDIADNTKKKILEKFNIREEDLDFSKMSNNTYFIRDCLK |
| | | NKNIDESEFTFEKNGKKLDPTDACFAREYKNKLSELTRKKIN |
| | | EKIWEIKKNSKEYHKISIYKKETIRYIVNKLIKQSKEKSECDDII |
| | | VNIEKLQIGGNFFGGRGKRDPGWNNFFLPKEENRWFINACH |
| | | KAFSELAPHKGIIVIESDPAYTSQTCPKCENCDKENRNGEKFK |
| | | CKKCNYEANADIDVATENLEKIAKNGRRLIKNFDQLGERLPG |
| | | AEMPGGARKRKPSKSLPKNGRGAGVGSEPELINQSPSQVIA |
| CasΦ.32 | 32 | VPDKKETPLVALCKKSFPGLRFKKHDSRQAGRILKSKGEGAA |
| | | VAFLEGKGGTTQPNFKPPVKCNIVAMSRPLEEWPIYKASVVI |
| | | QKYVYAQSYEEFKATDPGKSEAGLRAWLKATRVDTDGYFN |
| | | VQGLNLIFQNARATYEGVLKKVENRNSKKVAKIEQRNEHRA |
| | | ERGLPLLTLDEPETALDETGHLRHRPGINCSVFGYQHMKLKP |
| | | YVPGSIPGVTGYSRDPSTPIAACGVDRLEIPEGQPGYVPPWDR |
| | | ENLSVKKHRRKRASWARSRGGAIDDNMLLAVVRVADDWA |
| | | LLDLRGLLRNTQYRKLLDRSVPVTIESLLNLVTNDPTLSVVK |
| | | KPGKPVRYTATLIYKQGVVPVVKAKVVKGSYVSKMLDDTT |
| | | ETFSLVGVDLGVNNLIAANALRIRPGKCVERLQAFTLPEQTV |
| | | EDFFRFRKAYDKHQENLRLAAVRSLTAEQQAEVLALDTFGP |
| | | EQAKMQVCGHLGLSVDEVPWDKVNSRSSILSDLAKERGVD |
| | | DTLYMFPFFKGKGKKRKTEIRKRWDVNWAQHFRPQLTSETR |
| | | KALNEAKWEAERNSSKYHQLSIRKKELSRHCVNYVIRTAEK |
| | | RAQCGKVIVAVEDLHHSFRRGGKGSRKSGWGGFFAAKQEG |
| | | RWLMDALFGAFCDLAVHRGYRVIKVDPYNTSRTCPECGHC |
| | | DKANRDRVNREAFICVCCGYRGNADIDVAAYNIAMVAITGV |
| | | SLRKAARASVASTPLESLAAE |
| CasΦ.33 | 33 | MSKTKELNDYQEALARRLPGVRHQKSVRRAARLVYDRQGE |
| | | DAMVAFLDGKEVDEPYTLQPPAKCHILAVSRPIEEWPIARVT |
| | | MAVQEHVYALPVHEVEKSRPETTEGSRSAWFKNSGVSNHG |
| | | VTHAQTLNAILKNAYNVYNGVIKKVENRNAKKRDSLAAKN |
| | | KSRERKGLPHFKADPPELATDEQGYLLQPPSPNSSVYLVQQH |
| | | LRTPQIDLPSGYTGPVVDPRSPIPSLIPIDRLAIPPGQPGYVPLH |
| | | DREKLTSNKHRRMKLPKSLRAQGALPVCFRVFDDWAVVDG |
| | | RGLLRHAQYRRLAPKNVSIAELLELYTGDPVIDIKRNLMTFR |
| | | FAEAVVEVTARKIVEKYHNKYLLKLTEPKGKPVREIGLVSID |
| | | LNVQRLIALAIYRVHQTGESQLALSPCLHREILPAKGLGDFDK |
| | | YKSKFNQLTEEILTAAVQTLTSAQQEEYQRYVEESSHEAKAD |
| | | LCLKYSITPHELAWDKMTSSTQYISRWLRDHGWNASDFTQIT |
| | | KGRKKVERLWSDSRWAQELKPKLSNETRRKLEDAKHDLQR |
| | | ANPEWQRLAKRKQEYSRHLANTVLSMAREYTACETVVIAIE |
| | | NLPMKGGFVDGNGSRESGWDNFFTHKKENRWMIKDIHKAL |
| | | SDLAPNRGVHVLEVNPQYTSQTCPECGHRDKANRDPIQRERF |
| | | CCTHCGAQRHADLEVATHNIAMVATTGKSLTGKSLAPQRLQ |
| | | EAAE |
| CasΦ.41 | 34 | VLLSDRIQYTDPSAPIPAMTVVDRRKIKKGEPGYVPPFMRKN |
| | | LSTNKHRRMRLSRGQKEACALPVGLRLPDGKDGWDFIIFDG |
| | | RALLRACRRLRLEVTSMDDVLDKFTGDPRIQLSPAGETIVTC |
| | | MLKPQHTGVIQQKLITGKMKDRLVQLTAEAPIAMLTVDLGE |
| | | HNLVACGAYTVGQRRGKLQSERLEAFLLPEKVLADFEGYRR |
| | | DSDEHSETLRHEALKALSKRQQREVLDMLRTGADQARESLC |
| | | YKYGLDLQALPWDKMSSNSTFIAQHLMSLGFGESATHVRYR |
| | | PKRKASERTILKYDSRFAAEEKIKLTDETRRAWNEAIWECQR |
| | | ASQEFRCLSVRKLQLARAAVNWTLTQAKQRSRCPRVVVVV |
| | | EDLNVRFMHGGGKRQEGWAGFFKARSEKRWFIQALHKAYT |
| | | ELPTNRGIHVMEVNPARTSITCTKCGYCDPENRYGEDFHCRN |
| | | PKCKVRGGHVANADLDIATENLARVALSGPMPKAPKLK |
| CasΦ.34 | 35 | MTPSFGYQMIIVTPIHHASGAWATLRLLFLNPKTSGVMLGMT |
| | | KTKSAFALMREEVFPGLLFKSADLKMAGRKFAKEGREAAIE |
| | | YLRGKDEERPANFKPPAKGDIIAQSRPFDQWPIVQVSQAIQK |
| | | YIFGLTKAEFDATKTLLYGEGNHPTTESRRRWFEATGVPDFG |
| | | FTSAQGLNAIFSSALARYEGVIQKVENRNEKRLKKLSEKNQR |
| | | LVEEGHAVEAYVPETAFHTLESLKALSEKSLVPLDDLMDKID |
| | | RLAQPPGINPCLYGYQQVAPYIYDPENPRGVVLPDLYLGYCR |
| | | KPDDPITACPNRLDIPKGQPGYIPEHQRGQLKKHGRVRRFRY |
| | | TNPQAKARAKAQTAILAVLRIDEDWVVMDLRGLLRNVYFRE |
| | | VAAPGELTARTLLDTFTGCPVLNLRSNVVTFCYDIESKGALH |
| | | AEYVRKGWATRNKLLDLTKDGQSVALLSVDLGQRHPVAVM |
| | | ISRLKRDDKGDLSEKSIQVVSRTFADQYVDKLKRYRVQYDA |
| | | LRKEIYDAALVSLPPEQQAEIRAYEAFAPGDAKANVLSVMFQ |
| | | GEVSPDELPWDKMNTNTHYISDLYLRRGGDPSRVFFVPQPST |
| | | PKKNAKKPPAPRKPVKRTDENVSHMPEFRPHLSNETREAFQ |
| | | KAKWTMERGNVRYAQLSRFLNQIVREANNWLVSEAKKLTQ |
| | | CQTVVWAIEDLHVPFFHGKGKYHETWDGFFRQKKEDRWFV |
| | | NVFHKAISERAPNKGEYVMEVAPYRTSQRCPVCGFVDADNR |
| | | HGDHFKCLRCGVELHADLEVATWNIALVAVQGHGIAGPPRE |
| | | QSCGGETAGTARKGKNIKKNKGLADAVTVEAQDSEGGSKK |
| | | DAGTARNPVYIPSESQVNCPAP |
| CasΦ.35 | 36 | MKPKTPKPPKTPVAALIDKHFPGKRFRASYLKSVGKKLKNQ |
| | | GEDVAVRFLTGKDEERPPNFQPPAKSNIVAQSRPIEEWPIHKV |
| | | SVAVQEYVYGLTVAEKEACSDAGESSSSHAAWFAKTGVENF |
| | | GYTSVQGLNKIFPPTFNRFDGVIKKVENRNEKKRQKATRINE |
| | | AKRNKGQSEDPPEAEVKATDDAGYLLQPPGINHSVYGYQSIT |
| | | LCPYTAEKFPTIKLPEEYAGYHSNPDAPIPAGVPDRLAIPEGQ |
| | | PGHVPEEHRAGLSTKKHRRVRQWYAMANWKPKPKRTSKPD |
| | | YDRLAKARAQGALLIVIRIDEDWVVVDARGLLRNVRWRSLG |
| | | KREITPNELLDLFTGDPVLDLKRGVVTFTYAEGVVNVCSRST |
| | | TKGKQTKVLLDAMTAPRDGKKRQIGMVAVDLGQTNPIAAE |
| | | YSRVGKNAAGTLEATPLSRSTLPDELLREIALYRKAHDRLEA |
| | | QLREEAVLKLTAEQQAENARYVETSEEGAKLALANLGVDTS |
| | | TLPWDAMTGWSTCISDHLINHGGDTSAVFFQTIRKGTKKLET |
| | | IKRKDSSWADIVRPRLTKETREALNDFLWELKRSHEGYEKLS |
| | | KRLEELARRAVNHVVQEVKWLTQCQDIVIVIEDLNVRNFHG |
| | | GGKRGGGWSNFFTVKKENRWFMQALHKAFSDLAAHRGIPV |
| | | LEVYPARTSITCLGCGHCDPENRDGEAFVCQQCGATFHADLE |
| | | VATRNIARVALTGEAMPKAPAREQPGGAKKRGTSRRRKLTE |
| | | VAVKSAEPTIHQAKNQQLNGTSRDPVYKGSELPAL |
| CasΦ.43 | 37 | MSEITDLLKANFKGKTFKSADMRMAGRILKKSGAQAVIKYL |
| | | SDKGAVDPPDFRPPAKCNIIAQSRPFDEWPICKASMAIQQHIY |
| | | GLTKNEFDESSPGTSSASHEQWFAKTGVDTHGFTHVQGLNLI |
| | | FQHAKKRYEGVIKKVENYNEKERKKFEGINERRSKEGMPLL |
| | | EPRLRTAFGDDGKFAEKPGVNPSIYLYQQTSPRPYDKTKHPY |
| | | VHAPFELKEITTIPTQDDRLKIPFGAPGHVPEKHRSQLSMAKH |
| | | KRRRAWYALSQNKPRPPKDGSKGRRSVRDLADLKAASLAD |
| | | AIPLVSRVGFDWVVIDGRGLLRNLRWRKLAHEGMTVEEML |
| | | GFFSGDPVIDPRRNVATFIYKAEHATVKSRKPIGGAKRAREEL |
| | | LKATASSDGVIRQVGLISVDLGQTNPVAYEISRMHQANGELV |
| | | AEHLEYGLLNDEQVNSIQRYRAAWDSMNESFRQKAIESLSM |
| | | EAQDEIMQASTGAAKRTREAVLTMFGPNATLPWSRMSSNTT |
| | | CISDALIEVGKEEETNFVTSNGPRKRTDAQWAAYLRPRVNPE |
| | | TRALLNQAVWDLMKRSDEYERLSKRKLEMARQCVNFVVAR |
| | | AEKLTQCNNIGIVLENLVVRNFHGSGRRESGWEGFFEPKREN |
| | | RWFMQVLHKAFSDLAQHRGVMVFEVHPAYSSQTCPACRYV |
| | | DPKNRSSEDRERFKCLKCGRSFNADREVATFNIREIARTGVG |
| | | LPKPDCERSRGVQTTGTARNPGRSLKSNKNPSEPKRVLQSKT |
| | | RKKITSTETQNEPLATDLKT |
| CasΦ.44 | 38 | MTPKTESPLSALCKKHFPGKRFRTNYLKDAGKILKKHGEDA |
| | | VVAFLSDKQEDEPANFCPPAKVHILAQSRPFEDWPINLASKAI |
| | | QTYVYGLTADERKTCEPGTSKESHDRWFKETGVDHHGFTSV |
| | | QGLNLIFKHTLNRYDGVIKKVETRNEKRRSSVVRINEKKAAE |
| | | GLPLIAAEAEETAFGEDGRLLQPPGVNHSIYCFQQVSPQPYSS |
| | | KKHPQVVLPHAVQGVDPDAPIPVGRPNRLDIPKGQPGYVPE |
| | | WQRPHLSMKCKRVRMWYARANWRRKPGRRSVLNEARLKE |
| | | ASAKGALPIVLVIGDDWLVMDARGLLRSVFWRRVAKPGLSL |
| | | SELLNVTPTGLFSGDPVIDPKRGLVTFTSKLGVVAVHSRKPTR |
| | | GKKSKDLLLKMTKPTDDGMPRHVGMVAIDLGQTNPVAAEY |
| | | SRVVQSDAGTLKQEPVSRGVLPDDLLKDVARYRRAYDLTEE |
| | | SIRQEAIALLSEGHRAEVTKLDQTTANETKRLLVDRGVSESLP |
| | | WEKMSSNTTYISDCLVALGKTDDVFFVPKAKKGKKETGIAV |
| | | KRKDHGWSKLLRPRTSPEARKALNENQWAVKRASPEYERLS |
| | | RRKLELGRRCVNHIIQETKRWTQCEDIVVVLEDLNVGFFHGS |
| | | GKRPDGWDNFFVSKRENRWFIQVLHKAFGDLATHRGTHVIE |
| | | VHPARTSITCIKCGHCDAGNRDGESFVCLASACGDRRHADLE |
| | | VATRNVARVAITGERMPPSEQARDVQKAGGARKRKPSARN |
| | | VKSSYPAVEPAPASP |
| CasΦ.36 | 39 | MSDNKMKKLSKEEKPLTPLQILIRKYIDKSQYPSGFKTTIIKQ |
| | | AGVRIKSVKSEQDEINLANWIISKYDPTYIKRDFNPSAKCQIIA |
| | | TSRSVADFDIVKMSNKVQEIFFASSHLDKNVFDIGKSKSDHD |
| | | SWFERNNVDRGIYTYSNVQGMNLIFSNTKNTYLGVAVKAQN |
| | | KFSSKMKRIQDINNFRITNHQSPLPIPDEIKIYDDAGFLLNPPG |
| | | VNPNIFGYQSCLLKPLENKEIISKTSFPEYSRLPADMIEVNYKI |
| | | SNRLKFSNDQKGFIQFKDKLNLFKINSQELFSKRRRLSGQPIL |
| | | LVASFGDDWVVLDGRGLLRQVYYRGIAKPGSITISELLGFFT |
| | | GDPIVDPIRGVVSLGFKPGVLSQETLKTTSARIFAEKLPNLVL |
| | | NNNVGLMSIDLGQTNPVSYRLSEITSNMSVEHICSDFLSQDQI |
| | | SSIEKAKTSLDNLEEEIAIKAVDHLSDEDKINFANFSKLNLPED |
| | | TRQSLFEKYPELIGSKLDFGSMGSGTSYIADELIKFENKDAFY |
| | | PSGKKKFDLSFSRDLRKKLSDETRKSYNDALFLEKRTNDKYL |
| | | KNAKRRKQIVRTVANSLVSKIEELGLTPVINIENLAMSGGFFD |
| | | GRGKREKGWDNFFKVKKENRWVMKDFHKAFSELSPHHGVI |
| | | VIESPPYCTSVTCTKCNFCDKKNRNGHKFTCQRCGLDANAD |
| | | LDIATENLEKVAISGKRMPGSERSSDERKVAVARKAKSPKGK |
| | | AIKGVKCTITDEPALLSANSQDCSQSTS |
| CasΦ.37 | 40 | MALSLAEVRERHFKGLRFRSSYLKRAGKILKKEGEAACVAY |
| | | LTGKDEESPPNFKPPAKCDVVAQSRPFEEWPIVQASVAVQSY |
| | | VYGLTKEAFEAFNPGTTKQSHEACLAATGIDTCGYSNVQGL |
| | | NLIFRQAKNRYEGVITKVENRNKKAKKKLTRKNEWRQKNG |
| | | HSELPEAPEELTFNDEGRLLQPPGINPSLYTYQQISPTPWSPKD |
| | | SSILPPQYAGYERDPNAPIPFGVAKDRLTIASGCPGYIPEWMR |
| | | TAGEKTNPRTQKKFMHPGLSTRKNKRMRLPRSVRSAPLGAL |
| | | LVTIHLGEDWLVLDVRGLLRNARWRGVAPKDISTQGLLNLF |
| | | TGDPVIDTRRGVVTFTYKPETVGIHSRTWLYKGKQTKEVLEK |
| | | LTQDQTVALVAIDLGQTNPVSAAASRVSRSGENLSIETVDRF |
| | | FLPDELIKELRLYRMAHDRLEERIREESTLALTEAQQAEVRAL |
| | | EHVVRDDAKNKVCAAFNLDAASLPWDQMTSNTTYLSEAIL |
| | | AQGVSRDQVFFTPNPKKGSKEPVEVMRKDRAWVYAFKAKL |
| | | SEETRKAKNEALWALKRASPDYARLSKRREELCRRSVNMVI |
| | | NRAKKRTQCQVVIPVLEDLNIGFFHGSGKRLPGWDNFFVAK |
| | | KENRWLMNGLHKSFSDLAVHRGFYVFEVMPHRTSITCPACG |
| | | HCDSENRDGEAFVCLSCKRTYHADLDVATHNLTQVAGTGLP |
| | | MPEREHPGGTKKPGGSRKPESPQTHAPILHRTDYSESADRLG |
| | | S |
| CasΦ.45 | 41 | QAVIKYLSDKGAVDPPDFRPPAKCNIIAQSRPFDEWPICKASM |
| | | AIQQHIYGLTKNEFDESSPGTSSASHEQWFAKTGVDTHGFTH |
| | | VQGLNLIFQHAKKRYEGVIKKVENYNEKERKKFEGINERRSK |
| | | EGMPLLEPRLRTAFGDDGKFAEKPGVNPSIYLYQQTSPRPYD |
| | | KTKHPYVHAPFELKEITTIPTQDDRLKIPFGAPGHVPEKHRSQ |
| | | LSMAKHKRRRAWYALSQNKPRPPKDGSKGRRSVRDLADLK |
| | | AASLADAIPLVSRVGFDWVVIDGRGLLRNLRWRKLAHEGMT |
| | | VEEMLGFFSGDPVIDPRRNVATFIYKAEHATVKSRKPIGGAK |
| | | RAREELLKATASSDGVIRQVGLISVDLGQTNPVAYEISRMHQ |
| | | ANGELVAEHLEYGLLNDEQVNSIQRYRAAWDSMNESFRQK |
| | | AIESLSMEAQDEIMQASTGAAKRTREAVLTMFGPNATLPWS |
| | | RMSSNTTCISDALIEVGKEEETNFVTSNGPRKRTDAQWAAYL |
| | | RPRVNPETRALLNQAVWDLMKRSDEYERLSKRKLEMARQC |
| | | VNFVVARAEKLTQCNNIGIVLENLVVRNFHGSGRRESGWEG |
| | | FFEPKRENRWFMQVLHKAFSDLAQHRGVMVFEVHPAYSSQ |
| | | TCPACRYVDPKNRSSEDRERFKCLKCGRSFNADREVATFNIR |
| | | EIARTGVGLPKPDCERSRDVQTPGTARKSGRSLKSQDNLSEP |
| | | KRVLQSKTRKKITSTETQNEPLATDLKT |
| CasΦ.38 | 42 | MIKEQSELSKLIEKYYPGKKFYSNDLKQAGKHLKKSEHLTAK |
| | | ESEELTVEFLKSCKEKLYDFRPPAKALIISTSRPFEEWPIYKAS |
| | | ESIQKYIYSLTKEELEKYNISTDKTSQENFFKESLIDNYGFANV |
| | | SGLNLIFQHTKAIYDGVLKKVNNRNNKILKKYKRKIEEGIEID |
| | | SPELEKAIDESGHFINPPGINKNIYCYQQVSPTIFNSFKETKIICP |
| | | FNYKRNPNDIIQKGVIDRLAIPFGEPGYIPDHQRDKVNKHKK |
| | | RIRKYYKNNENKNKDAILAKINIGEDWVLFDLRGLLRNAYW |
| | | RKLIPKQGITPQQLLDMFSGDPVIDPIKNNITFIYKESIIPIHSESI |
| | | IKTKKSKELLEKLTKDEQIALVSIDLGQTNPVAARFSRLSSDL |
| | | KPEHVSSSFLPDELKNEICRYREKSDLLEIEIKNKAIKMLSQEQ |
| | | QDEIKLVNDISSEELKNSVCKKYNIDNSKIPWDKMNGFTTFIA |
| | | DEFINNGGDKSLVYFTAKDKKSKKEKLVKLSDKKIANSFKPK |
| | | ISKETREILNKITWDEKISSNEYKKLSKRKLEFARRATNYLIN |
| | | QAKKATRLNNVVLVVEDLNSKFFHGSGKREDGWDNFFIPKK |
| | | ENRWFIQALHKSLTDVSIHRGINVIEVRPERTSITCPKCGCCD |
| | | KENRKGEDFKCIKCDSVYHADLEVATFNIEKVAITGESMPKP |
| | | DCERLGGEESIG |
| CasΦ.39 | 43 | VAFLDGKEVDEPYTLQPPAKCHILAVSRPIEEWPIARVTMAV |
| | | QEHVYALPVHEVEKSRPETTEGSRSAWFKNSGVSNHGVTHA |
| | | QTLNAILKNAYNVYNGVIKKVENRNAKKRDSLAAKNKSRER |
| | | KGLPHFKADPPELATDEQGYLLQPPSPNSSVYLVQQHLRTPQ |
| | | IDLPSGYTGPVVDPRSPIPSLIPIDRLAIPPGQPGYVPLHDREKL |
| | | TSNKHRRMKLPKSLRAQGALPVCFRVFDDWAVVDGRGLLR |
| | | HAQYRRLAPKNVSIAELLELYTGDPVIDIKRNLMTFRFAEAV |
| | | VEVTARKIVEKYHNKYLLKLTEPKGKPVREIGLVSIDLNVQR |
| | | LIALAIYRVHQTGESQLALSPCLHREILPAKGLGDFDKYKSKF |
| | | NQLTEEILTAAVQTLTSAQQEEYQRYVEESSHEAKADLCLKY |
| | | SITPHELAWDKMTSSTQYISRWLRDHGWNASDFTQITKGRK |
| | | KVERLWSDSRWAQELKPKLSNETRRKLEDAKHDLQRANPE |
| | | WQRLAKRKQEYSRHLANTVLSMAREYTACETVVIAIENLPM |
| | | KGGFVDGNGSRESGWDNFFTHKKENRWMIKDIHKALSDLAP |
| | | NRGVHVLEVNPQYTSQTCPECGHRDKANRDPIQRERFCCTH |
| | | CGAQRHADLEVATHNIAMVATTGKSLTGKSLAPQRLQ |
| CasΦ.42 | 44 | LEIPEGEPGHVPWFQRMDIPEGQIGHVNKIQRFNFVHGKNSG |
| | | KVKFSDKTGRVKRYHHSKYKDATKPYKFLEESKKVSALDSI |
| | | LAIITIGDDWVVFDIRGLYRNVFYRELAQKGLTAVQLLDLFT |
| | | GDPVIDPKKGIITFSYKEGVVPVFSQKIVSRFKSRDTLEKLTSQ |
| | | GPVALLSVDLGQNEPVAARVCSLKNINDKIALDNSCRIPFLD |
| | | DYKKQIKDYRDSLDELEIKIRLEAINSLDVNQQVEIRDLDVFS |
| | | ADRAKASTVDMFDIDPNLISWDSMSDARFSTQISDLYLKNGG |
| | | DESRVYFEINNKRIKRSDYNISQLVRPKLSDSTRKNLNDSIWK |
| | | LKRTSEEYLKLSKRKLELSRAVVNYTIRQSKLLSGINDIVIILE |
| | | DLDVKKKFNGRGIRDIGWDNFFSSRKENRWFIPAFHKSFSEL |
| | | SSNRGLCVIEVNPAWTSATCPDCGFCSKENRDGINFTCRKCG |
| | | VSYHADIDVATLNIARVAVLGKPMSGPADRERLGGTKKPRV |
| | | ARSRKDMKRKDISNGTVEVMVTA |
| CasΦ.46 | 45 | IPSFGYLDRLKIAKGQPGYIPEWQRETINPSKKVRRYWATNH |
| | | EKIRNAIPLVVFIGDDWVIIDGRGLLRDARRRKLADKNTTIEQ |
| | | LLEMVSNDPVIDSTRGIATLSYVEGVVPVRSFIPIGEKKGREY |
| | | LEKSTQKESVTLLSVDIGQINPVSCGVYKVSNGCSKIDFLDKF |
| | | FLDKKHLDAIQKYRTLQDSLEASIVNEALDEIDPSFKKEYQNI |
| | | NSQTSNDVKKSLCTEYNIDPEAISWQDITAHSTLISDYLIDNNI |
| | | TNDVYRTVNKAKYKTNDFGWYKKFSAKLSKEAREALNEKI |
| | | WELKIASSKYKKLSVRKKEIARTIANDCVKRAETYGDNVVV |
| | | AMESLTKNNKVMSGRGKRDPGWHNLGQAKVENRWFIQAIS |
| | | SAFEDKATHHGTPVLKVNPAYTSQTCPSCGHCSKDNRSSKD |
| | | RTIFVCKSCGEKFNADLDVATYNIAHVAFSGKKLSPPSEKSSA |
| | | TKKPRSARKSKKSRKS |
| CasΦ.47 | 46 | SPIEKLLNGLLVKITFGNDWIICDARGLLDNVQKGIIHKSYFT |
| | | NKSSLVDLIDLFTCNPIVNYKNNVVTFCYKEGVVDVKSFTPI |
| | | KSGPKTQENLIKKLKYSRFQNEKDACVLGVGVDVGVTNPFA |
| | | INGFKMPVDESSEWVMLNEPLFTIETSQAFREEIMAYQQRTD |
| | | EMNDQFNQQSIDLLPPEYKVEFDNLPEDINEVAKYNLLHTLN |
| | | IPNNFLWDKMSNTTQFISDYLIQIGRGTETEKTITTKKGKEKIL |
| | | TIRDVNWFNTFKPKISEETGKARTEIKRDLQKNSDQFQKLAK |
| | | SREQSCRTWVNNVTEEAKIKSGCPLIIFVIEALVKDNRVFSGK |
| | | GHRAIGWHNFGKQKNERRWWVQAIHKAFQEQGVNHGYPVI |
| | | LCPPQYTSQTCPKCNHVDRDNRSGEKFKCLKYGWIGNADLD |
| | | VGAYNIARVAITGKALSKPLEQKKIKKAKNKT |
| CasΦ.48 | 47 | LLDNVQKGIIHKSYFTNKSSLVDLIDLFTCNPIVNYKNNVVTF |
| | | CYKEGVVDVKSFTPIKSGPKTQENLIKKLKYSRFQNEKDACV |
| | | LGVGVDVGVTNPFAINGFKMPVDESSEWVMLNEPLFTIETSQ |
| | | AFREEIMAYQQRTDEMNDQFNQQSIDLLPPEYKVEFDNLPED |
| | | INEVAKYNLLHTLNIPNNFLWDKMSNTTQFISDYLIQIGRGTE |
| | | TEKTITTKKGKEKILTIRDVNWFNTFKPKISEETGKARTEIKR |
| | | DLQKNSDQFQKLAKSREQSCRTWVNNVTEEAKIKSGCPLIIF |
| | | VIEALVKDNRVFSGKGHRAIGWHNFGKQKNERRWWVQAIH |
| | | KAFQEQGVNHGYPVILCPPQYTSQTCPKCNHVDRDNRSGEK |
| | | FKCLKYGWIGNADLDVGAYNIARVAITGKALSKPLEQKKIK |
| | | KAKNKT |
| CasΦ.49 | 105 | MIKPTVSQFLTPGFKLIRNHSRTAGLKLKNEGEEACKKFVRE |
| | | NEIPKDECPNFQGGPAIANIIAKSREFTEWEIYQSSLAIQEVIFT |
| | | LPKDKLPEPILKEEWRAQWLSEHGLDTVPYKEAAGLNLIIKN |
| | | AVNTYKGVQVKVDNKNKNNLAKINRKNEIAKLNGEQEISFE |
| | | EIKAFDDKGYLLQKPSPNKSIYCYQSVSPKPFITSKYHNVNLP |
| | | EEYIGYYRKSNEPIVSPYQFDRLRIPIGEPGYVPKWQYTFLSK |
| | | KENKRRKLSKRIKNVSPILGIICIKKDWCVFDMRGLLRTNHW |
| | | KKYHKPTDSINDLFDYFTGDPVIDTKANVVRFRYKMENGIV |
| | | NYKPVREKKGKELLENICDQNGSCKLATVDVGQNNPVAIGL |
| | | FELKKVNGELTKTLISRHPTPIDFCNKITAYRERYDKLESSIKL |
| | | DAIKQLTSEQKIEVDNYNNNFTPQNTKQIVCSKLNINPNDLP |
| | | WDKMISGTHFISEKAQVSNKSEIYFTSTDKGKTKDVMKSDY |
| | | KWFQDYKPKLSKEVRDALSDIEWRLRRESLEFNKLSKSREQ |
| | | DARQLANWISSMCDVIGIENLVKKNNFFGGSGKREPGWDNF |
| | | YKPKKENRWWINAIHKALTELSQNKGKRVILLPAMRTSITCP |
| | | KCKYCDSKNRNGEKFNCLKCGIELNADIDVATENLATVAITA |
| | | QSMPKPTCERSGDAKKPVRARKAKAPEFHDKLAPSYTVVLR |
| | | EAVKRPAATKKAGQAKKKKEF |
| | | (Underlined sequence is Nuclear Localization Signal; SEQ ID |
| | | NO: 106) |
| CasΦ.12 | 107 | SNAPKKKRKVGIHGVPAAMIKPTVSQFLTPGFKLIRNHSRT |
| with NLS | | AGLKLKNEGEEACKKFVRENEIPKDECPNFQGGPAIANIIAKS |
| Signals | | REFTEWEIYQSSLAIQEVIFTLPKDKLPEPILKEEWRAQWLSE |
| | | HGLDTVPYKEAAGLNLIIKNAVNTYKGVQVKVDNKNKNNL |
| | | AKINRKNEIAKLNGEQEISFEEIKAFDDKGYLLQKPSPNKSIY |
| | | CYQSVSPKPFITSKYHNVNLPEEYIGYYRKSNEPIVSPYQFDR |
| | | LRIPIGEPGYVPKWQYTFLSKKENKRRKLSKRIKNVSPILGIICI |
| | | KKDWCVFDMRGLLRTNHWKKYHKPTDSINDLFDYFTGDPVI |
| | | DTKANVVRFRYKMENGIVNYKPVREKKGKELLENICDQNGS |
| | | CKLATVDVGQNNPVAIGLFELKKVNGELTKTLISRHPTPIDFC |
| | | NKITAYRERYDKLESSIKLDAIKQLTSEQKIEVDNYNNNFTPQ |
| | | NTKQIVCSKLNINPNDLPWDKMISGTHFISEKAQVSNKSEIYF |
| | | TSTDKGKTKDVMKSDYKWFQDYKPKLSKEVRDALSDIEWR |
| | | LRRESLEFNKLSKSREQDARQLANWISSMCDVIGIENLVKKN |
| | | NFFGGSGKREPGWDNFYKPKKENRWWINAIHKALTELSQNK |
| | | GKRVILLPAMRTSITCPKCKYCDSKNRNGEKFNCLKCGIELN |
| | | ADIDVATENLATVAITAQSMPKPTCERSGDAKKPVRARKAK |
| | | APEFHDKLAPSYTVVLREAVKRPAATKKAGQAKKKKEF |
| | | (Underlined sequences Nuclear Localization Signals; SEQ ID |
| | | NO: 112 and 106) |

In some embodiments, any of the programmable CasΦ nucleases of the present disclosure (e.g., any one of SEQ TD NO: 1 to 47, 105, or 107, or fragments or variants thereof) may include a nuclear localization signal (NLS). In some cases, one or more NLS are fused or linked to the N-terminus of the programmable CasΦ nuclease. In some embodiments, one or more NLS are fused or linked to the C-terminus of the programmable CasΦ nuclease. In some embodiments, one or more NLS are fused or linked to the N-terminus and the C-terminus of the programmable CasΦ nuclease. In some embodiments, the link between the NLS and the programmable CasΦ nuclease comprises a tag. In some cases, said NLS may have a sequence of KRPAATKKAGQAKKKKEF (SEQ ID NO: 106). The NLS can be selected to match the cell type of interest, for example several NLSs are known to be functional in different types of eukaryotic cell e.g. in mammalian cells. Suitable NLSs include the SV40 large T antigen NLS (PKKKRKV, SEQ ID NO: 110) and the c-Myc NLS (PAAKRVKLD,SEQ ID NO: 111). In some embodiments, an NLS may be the SV40 large T antigen NLS or the c-Myc NLS. NLSs that are functional in plant cells are described in Chang et al., (Plant Signal Behav. 2013 October; 8(10):e25976). In some embodiments, an NLS sequence can be selected from the following consensus sequences: KR(K/R)R, K(K/R)RK; (P/R)XXKR(\hat{ }de)(K/R); KRX(W/F/Y)XXAF (SEQ ID NO: 2489); (R/P)XXKR(K/R)(\hat{ }de); LGKR(K/R)(W/F/Y) (SEQ ID NO: 2490); KRX10-12K(KR)(KR) or KRX10-12K(KR)X(K/R).

In some embodiments, the nucleoplasmin NLS (KRPAATKKAGQAKKKKEF (SEQ ID NO: 106)) is linked or fused to the C-terminus of the programmable CasΦ nuclease. In some embodiments, the SV40 NLS (PKKKRKVGIHGVPAA) (SEQ ID NO: 112) is linked or fused to the N-terminus of the programmable CasΦ nuclease. In preferred embodiments, the nucleoplasmin NLS (SEQ ID NO: 106) is linked or fused to the C-terminus of the programmable CasΦ nuclease and the SV40 NLS (SEQ ID NO: 112) is linked or fused to the N-terminus of the programmable CasΦ nuclease.

In some embodiments, the CasΦ nuclease comprises more than 200 amino acids, more than 300 amino acids, more than 400 amino acids. In some embodiments, the CasΦ nuclease comprises less than 1500 amino acids, less than 1000 amino acids or less than 900 amino acids. In some embodiments, the CasΦ nuclease comprises between 200 and 1500 amino acids, between 300 and 1000 amino acids, or between 400 and 900 amino acids. In preferred embodiments, the CasΦ nuclease comprises between 400 and 900 amino acids.

“Percent identity” and “% identity” can refer to the extent to which two sequences (nucleotide or amino acid) have the same residue at the same positions in an alignment. For example, “an amino acid sequence is X % identical to SEQ ID NO: Y” can refer to % identity of the amino acid sequence to SEQ ID NO: Y and is elaborated as X % of residues in the amino acid sequence are identical to the residues of sequence disclosed in SEQ ID NO: Y. Generally, computer programs can be employed for such calculations. Illustrative programs that compare and align pairs of sequences, include ALIGN (Myers and Miller, Comput Appl Biosci. 1988 March; 4(1):11-7), FASTA (Pearson and Lipman, Proc Natl Acad Sci USA. 1988 April; 85(8):2444-8; Pearson, Methods Enzymol. 1990; 183:63-98) and gapped BLAST (Altschul et al., Nucleic Acids Res. 1997 Sep. 1; 25(17):3389-40), BLASTP, BLASTN, or GCG (Devereux et al., Nucleic Acids Res. 1984 Jan. 11; 12(1 Pt 1):387-95).

A CasΦ polypeptide or a variant thereof can comprise at least 70%, at least 75%, at least 80%, at least 85%, at least 90%, at least 92%, at least 95%, at least 97%, at least 98%, at least 99%, or 100% sequence identity with any one of SEQ ID NO: 1 to SEQ ID NO: 47, SEQ ID NO. 105, and SEQ ID NO: 107.

A programmable nuclease or nickase of the present disclosure can comprise at least 70%, at least 75%, at least 80%, at least 85%, at least 90%, at least 92%, at least 95%, at least 97%, at least 98%, at least 99%, or 100% sequence identity with any one of SEQ ID NO: 1 to SEQ ID NO: 47, SEQ ID NO. 105, and SEQ ID NO: 107.

Compositions and methods of the disclosure can comprise a programmable nuclease comprising at least 70%, at least 75%, at least 80%, at least 85%, at least 90%, at least 92%, at least 95%, at least 97%, at least 98%, at least 99%, or 100% sequence identity to SEQ ID NO: 2.

Compositions and methods of the disclosure can comprise a programmable nuclease comprising at least 70%, at least 75%, at least 80%, at least 85%, at least 90%, at least 92%, at least 95%, at least 97%, at least 98%, at least 99%, or 100% sequence identity to SEQ ID NO: 4.

Compositions and methods of the disclosure can comprise a programmable nuclease comprising at least 70%, at least 75%, at least 80%, at least 85%, at least 90%, at least 92%, at least 95%, at least 97%, at least 98%, at least 99%, or 100% sequence identity to SEQ ID NO: 11.

Compositions and methods of the disclosure can comprise a programmable nuclease comprising at least 70%, at least 75%, at least 80%, at least 85%, at least 90%, at least 92%, at least 95%, at least 97%, at least 98%, at least 99%, or 100% sequence identity to SEQ ID NO: 17.

Compositions and methods of the disclosure can comprise a programmable nuclease comprising at least 70%, at least 75%, at least 80%, at least 85%, at least 90%, at least 92%, at least 95%, at least 97%, at least 98%, at least 99%, or 100% sequence identity to SEQ ID NO: 18.

Compositions and methods of the disclosure can comprise a programmable polypeptide or nuclease comprising at least 70%, at least 75%, at least 80%, at least 85%, at least 90%, at least 92%, at least 95%, at least 97%, at least 98%, at least 99%, or 100% sequence identity to SEQ ID NO: 12.

Compositions and methods of the disclosure can comprise a programmable polypeptide or nuclease comprising at least 70%, at least 75%, at least 80%, at least 85%, at least 90%, at least 92%, at least 95%, at least 97%, at least 98%, at least 99%, or 100% sequence identity to SEQ ID NO: 105.

Compositions and methods of the disclosure can comprise a programmable polypeptide or nuclease comprising at least 70%, at least 75%, at least 80%, at least 85%, at least 90%, at least 92%, at least 95%, at least 97%, at least 98%, at least 99%, or 100% sequence identity to SEQ ID NO: 107.

In some embodiments, the programmable nuclease comprises a sequence with at least 70% identity to SEQ ID NO: 2. In some embodiments, the programmable nuclease comprises a sequence with at least 75% identity to SEQ ID NO: 2. In some embodiments, the programmable nuclease comprises a sequence with at least 80% identity to SEQ ID NO: 2. In some embodiments, the programmable nuclease comprises a sequence with at least 85% identity to SEQ ID NO: 2. In some embodiments, the programmable nuclease comprises a sequence with at least 90% identity to SEQ ID NO: 2. In some embodiments, the programmable nuclease comprises a sequence with at least 92% identity to SEQ ID NO: 2. In some embodiments, the programmable nuclease comprises a sequence with at least 95% identity to SEQ ID NO: 2. In some embodiments, the programmable nuclease comprises a sequence with at least 97% identity to SEQ ID NO: 2. In some embodiments, the programmable nuclease comprises a sequence with at least 98% identity to SEQ ID NO: 2. In some embodiments, the programmable nuclease comprises a sequence with at least 99% identity to SEQ ID NO: 2. In some embodiments, the programmable nuclease comprises a sequence of SEQ ID NO: 2.

In some embodiments, the programmable nuclease comprises a sequence with at least 70% identity to SEQ ID NO: 4. In some embodiments, the programmable nuclease comprises a sequence with at least 75% identity to SEQ ID NO: 4. In some embodiments, the programmable nuclease comprises a sequence with at least 80% identity to SEQ ID NO: 4. In some embodiments, the programmable nuclease comprises a sequence with at least 85% identity to SEQ ID NO: 4. In some embodiments, the programmable nuclease comprises a sequence with at least 90% identity to SEQ ID NO: 4. In some embodiments, the programmable nuclease comprises a sequence with at least 92% identity to SEQ ID NO: 4. In some embodiments, the programmable nuclease comprises a sequence with at least 95% identity to SEQ ID NO: 4. In some embodiments, the programmable nuclease comprises a sequence with at least 97% identity to SEQ ID NO: 4. In some embodiments, the programmable nuclease comprises a sequence with at least 98% identity to SEQ ID NO: 4. In some embodiments, the programmable nuclease comprises a sequence with at least 99% identity to SEQ ID NO: 4. In some embodiments, the programmable nuclease comprises a sequence of SEQ ID NO: 4.

In some embodiments, the programmable nuclease comprises a sequence with at least 70% identity to SEQ ID NO: 11. In some embodiments, the programmable nuclease comprises a sequence with at least 75% identity to SEQ ID NO: 11. In some embodiments, the programmable nuclease comprises a sequence with at least 80% identity to SEQ ID NO: 11. In some embodiments, the programmable nuclease comprises a sequence with at least 85% identity to SEQ ID NO: 11. In some embodiments, the programmable nuclease comprises a sequence with at least 90% identity to SEQ ID NO: 11. In some embodiments, the programmable nuclease comprises a sequence with at least 92% identity to SEQ ID NO: 11. In some embodiments, the programmable nuclease comprises a sequence with at least 95% identity to SEQ ID NO: 11. In some embodiments, the programmable nuclease comprises a sequence with at least 97% identity to SEQ ID NO: 11. In some embodiments, the programmable nuclease comprises a sequence with at least 98% identity to SEQ ID NO: 11. In some embodiments, the programmable nuclease comprises a sequence with at least 99% identity to SEQ ID NO: 11. In some embodiments, the programmable nuclease comprises a sequence of SEQ ID NO: 11.

In some embodiments, the programmable nuclease comprises a sequence with at least 70% identity to SEQ ID NO: 12. In some embodiments, the programmable nuclease comprises a sequence with at least 75% identity to SEQ ID NO: 12. In some embodiments, the programmable nuclease comprises a sequence with at least 80% identity to SEQ ID NO: 12. In some embodiments, the programmable nuclease comprises a sequence with at least 85% identity to SEQ ID NO: 12. In some embodiments, the programmable nuclease comprises a sequence with at least 90% identity to SEQ ID NO: 12. In some embodiments, the programmable nuclease comprises a sequence with at least 92% identity to SEQ ID NO: 12. In some embodiments, the programmable nuclease comprises a sequence with at least 95% identity to SEQ ID NO: 12. In some embodiments, the programmable nuclease comprises a sequence with at least 97% identity to SEQ ID NO: 12. In some embodiments, the programmable nuclease comprises a sequence with at least 98% identity to SEQ ID NO: 12. In some embodiments, the programmable nuclease comprises a sequence with at least 99% identity to SEQ ID NO: 12. In some embodiments, the programmable nuclease comprises a sequence of SEQ ID NO: 12.

In some embodiments, the programmable nuclease comprises a sequence with at least 70% identity to SEQ ID NO: 17. In some embodiments, the programmable nuclease comprises a sequence with at least 75% identity to SEQ ID NO: 17. In some embodiments, the programmable nuclease comprises a sequence with at least 80% identity to SEQ ID NO: 17. In some embodiments, the programmable nuclease comprises a sequence with at least 85% identity to SEQ ID NO: 17. In some embodiments, the programmable nuclease comprises a sequence with at least 90% identity to SEQ ID NO: 17. In some embodiments, the programmable nuclease comprises a sequence with at least 92% identity to SEQ ID NO: 17. In some embodiments, the programmable nuclease comprises a sequence with at least 95% identity to SEQ ID NO: 17. In some embodiments, the programmable nuclease comprises a sequence with at least 97% identity to SEQ ID NO: 17. In some embodiments, the programmable nuclease comprises a sequence with at least 98% identity to SEQ ID NO: 17. In some embodiments, the programmable nuclease comprises a sequence with at least 99% identity to SEQ ID NO: 17. In some embodiments, the programmable nuclease comprises a sequence of SEQ ID NO: 17.

In some embodiments, the programmable nuclease comprises a sequence with at least 70% identity to SEQ ID NO: 18. In some embodiments, the programmable nuclease comprises a sequence with at least 75% identity to SEQ ID NO: 18. In some embodiments, the programmable nuclease comprises a sequence with at least 80% identity to SEQ ID NO: 18. In some embodiments, the programmable nuclease comprises a sequence with at least 85% identity to SEQ ID NO: 18. In some embodiments, the programmable nuclease comprises a sequence with at least 90% identity to SEQ ID NO: 18. In some embodiments, the programmable nuclease comprises a sequence with at least 92% identity to SEQ ID NO: 18. In some embodiments, the programmable nuclease comprises a sequence with at least 95% identity to SEQ ID NO: 18. In some embodiments, the programmable nuclease comprises a sequence with at least 97% identity to SEQ ID NO: 18. In some embodiments, the programmable nuclease comprises a sequence with at least 98% identity to SEQ ID NO: 18. In some embodiments, the programmable nuclease comprises a sequence with at least 99% identity to SEQ ID NO: 18. In some embodiments, the programmable nuclease comprises a sequence of SEQ ID NO: 18.

In some embodiments, the programmable nuclease comprises a sequence with at least 70% identity to SEQ ID NO: 105. In some embodiments, the programmable nuclease comprises a sequence with at least 75% identity to SEQ ID NO: 105. In some embodiments, the programmable nuclease comprises a sequence with at least 80% identity to SEQ ID NO: 105. In some embodiments, the programmable nuclease comprises a sequence with at least 85% identity to SEQ ID NO: 105. In some embodiments, the programmable nuclease comprises a sequence with at least 90% identity to SEQ ID NO: 105. In some embodiments, the programmable nuclease comprises a sequence with at least 92% identity to SEQ ID NO: 105. In some embodiments, the programmable nuclease comprises a sequence with at least 95% identity to SEQ ID NO: 105. In some embodiments, the programmable nuclease comprises a sequence with at least 97% identity to SEQ ID NO: 105. In some embodiments, the programmable nuclease comprises a sequence with at least 98% identity to SEQ ID NO: 105. In some embodiments, the programmable nuclease comprises a sequence with at least 99% identity to SEQ ID NO: 105. In some embodiments, the programmable nuclease comprises a sequence of SEQ ID NO: 105.

In some embodiments, the programmable nuclease comprises a sequence with at least 70% identity to the N-terminal 717 amino acid residues of SEQ ID NO: 105. In some embodiments, the programmable nuclease comprises a sequence with at least 75% identity to the N-terminal 717 amino acid residues of SEQ ID NO: 105. In some embodiments, the programmable nuclease comprises a sequence with at least 80% identity to the N-terminal 717 amino acid residues of SEQ ID NO: 105. In some embodiments, the programmable nuclease comprises a sequence with at least 85% identity to the N-terminal 717 amino acid residues of SEQ ID NO: 105. In some embodiments, the programmable nuclease comprises a sequence with at least 90% identity to SEQ ID NO: 105. In some embodiments, the programmable nuclease comprises a sequence with at least 95% identity to the N-terminal 717 amino acid residues of SEQ ID NO: 105. In some embodiments, the programmable nuclease comprises a sequence with at least 98% identity to the N-terminal 717 amino acid residues of SEQ ID NO: 105. In some embodiments, the programmable nuclease comprises a sequence with at least 99% identity to the N-terminal 717 amino acid residues of SEQ ID NO: 105. In some embodiments, the programmable nuclease comprises a sequence of the N-terminal 717 amino acid residues of SEQ ID NO: 105.

In some embodiments, the programmable nuclease comprises a sequence with at least 70% identity to SEQ ID NO: 106. In some embodiments, the programmable nuclease comprises a sequence with 75% identity to SEQ ID NO: 106. In some embodiments, the programmable nuclease comprises a sequence with at least 80% identity to SEQ ID NO: 106. In some embodiments, the programmable nuclease comprises a sequence with at least 85% identity to SEQ ID NO: 106. In some embodiments, the programmable nuclease comprises a sequence with at least 90% identity to SEQ ID NO: 105. In some embodiments, the programmable nuclease comprises a sequence with at least 95% identity to SEQ ID NO: 106. In some embodiments, the programmable nuclease comprises a sequence with at least 98% identity to SEQ ID NO: 106. In some embodiments, the programmable nuclease comprises a sequence with at least 99% identity to SEQ ID NO: 106. In some embodiments, the programmable nuclease comprises a sequence of SEQ ID NO: 106.

In some embodiments, the programmable nuclease comprises a sequence with at least 70% identity to SEQ ID NO: 107. In some embodiments, the programmable nuclease comprises a sequence with at least 75% identity to SEQ ID NO: 107. In some embodiments, the programmable nuclease comprises a sequence with at least 80% identity to SEQ ID NO: 107. In some embodiments, the programmable nuclease comprises a sequence with at least 85% identity to SEQ ID NO: 107. In some embodiments, the programmable nuclease comprises a sequence with at least 90% identity to SEQ ID NO: 107. In some embodiments, the programmable nuclease comprises a sequence with at least 95% identity to SEQ ID NO: 107. In some embodiments, the programmable nuclease comprises a sequence with at least 98% identity to SEQ ID NO: 107. In some embodiments, the programmable nuclease comprises a sequence with at least 99% identity to SEQ ID NO: 107. In some embodiments, the programmable nuclease comprises a sequence of SEQ ID NO: 107.

The programmable nucleases disclosed herein can be codon optimized for expression in a specific cell, for example, a bacterial cell, a plant cell, a eukaryotic cell, an animal cell, a mammalian cell, or a human cell. In some embodiments, the programmable nuclease is codon optimized for a human cell.

The programmable nucleases presented in TABLE 1 or variants or fragments thereof comprising at least 70%, at least 75%, at least 80%, at least 85%, at least 90%, at least 92%, at least 95%, at least 97%, at least 99%, or 100% sequence identity with any one of SEQ ID NO: 1-SEQ ID NO: 47, SEQ ID NO. 105, and SEQ ID NO: 107 can comprise nicking activity. Compositions and methods of the disclosure can comprise a programmable nickase comprising at least 70%, at least 75%, at least 80%, at least 85%, at least 90%, at least 92%, at least 95%, at least 97%, at least 99%, or 100% sequence identity to any one of SEQ ID NO: 1-SEQ ID NO: 47, SEQ ID NO. 105, and SEQ ID NO: 107. Compositions and methods of the disclosure can comprise a programmable nickase comprising at least 70%, at least 75%, at least 80%, at least 85%, at least 90%, at least 92%, at least 95%, at least 97%, at least 99%, or 100% sequence identity to SEQ ID NO: 2. Compositions and methods of the disclosure can comprise a programmable nickase comprising at least 70%, at least 75%, at least 80%, at least 85%, at least 90%, at least 92%, at least 95%, at least 97%, at least 99%, or 100% sequence identity to SEQ ID NO: 4. Compositions and methods of the disclosure can comprise a programmable nickase comprising at least 70%, at least 75%, at least 80%, at least 85%, at least 90%, at least 92%, at least 95%, at least 97%, at least 99%, or 100% sequence identity to SEQ ID NO: 11. Compositions and methods of the disclosure can comprise a programmable nickase comprising at least 70%, at least 75%, at least 80%, at least 85%, at least 90%, at least 92%, at least 95%, at least 97%, at least 99%, or 100% sequence identity to SEQ ID NO: 17. Compositions and methods of the disclosure can comprise a programmable nuclease comprising at least 70%, at least 75%, at least 80%, at least 85%, at least 90%, at least 92%, at least 95%, at least 97%, at least 99%, or 100% sequence identity to SEQ ID NO: 18.

The programmable nucleases presented in TABLE 1 or variants thereof comprising at least 70%, at least 75%, at least 80%, at least 85%, at least 90%, at least 92%, at least 95%, at least 97%, at least 99%, or 100% sequence identity with any one of SEQ ID NO: 1-SEQ ID NO: 47, SEQ ID NO. 105, and SEQ ID NO: 107 can comprise double-strand DNA cleavage activity. Compositions and methods of the disclosure can comprise a programmable nuclease capable of introducing a double-strand break in a target DNA sequence and comprising at least 70%, at least 75%, at least 80%, at least 85%, at least 90%, at least 92%, at least 95%, at least 97%, at least 99%, or 100% sequence identity to any one of SEQ ID NO: 1-SEQ ID NO: 47, SEQ ID NO. 105, and SEQ ID NO: 107. Compositions and methods of the disclosure can comprise a programmable nuclease with double-strand DNA cleaving activity and comprising at least 70%, at least 75%, at least 80%, at least 85%, at least 90%, at least 92%, at least 95%, at least 97%, at least 99%, or 100% sequence identity to SEQ ID NO: 12. Compositions and methods of the disclosure can comprise a programmable nuclease with double-strand DNA cleaving activity and comprising at least 70%, at least 75%, at least 80%, at least 85%, at least 90%, at least 92%, at least 95%, at least 97%, at least 99%, or 100% sequence identity to SEQ ID NO: 2. Compositions and methods of the disclosure can comprise a programmable nickase comprising at least 70%, at least 75%, at least 80%, at least 85%, at least 90%, at least 92%, at least 95%, at least 97%, at least 99%, or 100% sequence identity to SEQ ID NO: 4. Compositions and methods of the disclosure can comprise a programmable nuclease with double-strand DNA cleaving activity and comprising at least 70%, at least 75%, at least 80%, at least 85%, at least 90%, at least 92%, at least 95%, at least 97%, at least 99%, or 100% sequence identity to SEQ ID NO: 11.

The programmable nucleases presented in TABLE 1 or variants thereof comprising at least 70%, at least 75%, at least 80%, at least 85%, at least 90%, at least 92%, at least 95%, at least 97%, at least 99%, or 100% sequence identity with any one of SEQ ID NO: 1-SEQ ID NO: 47 and SEQ ID NO. 105 can comprise nickase activity and double-strand DNA cleavage activity. The ratio of the nickase activity and double-strand DNA cleavage activity can be modulated depending on the reaction conditions including for example, RNP complexing temperature, the crRNA repeat sequence in the guide nucleic acid. In some embodiments, nickase activity is reduced when RNP complexing temperature is room temperature, for example 20 to 22° C., compared to when RNP complexing temperature is 37° C. In some embodiments, the double-strand DNA cleavage activity is insensitive to RNP complexing at 37° C. compared to room temperature, or the double-strand DNA cleavage activity is reduced by 10%, 20% or 30% when complexed with a guide RNA at room temperature as compared to when complexed at 37° C. In a preferred embodiment, double-strand cleavage activity is similar when the RNP complexing temperature is room temperature and 37° C.

The programmable nucleases presented in TABLE 1 or variants thereof comprising at least 70%, at least 75%, at least 80%, at least 85%, at least 90%, at least 92%, at least 95%, at least 97%, at least 99%, or 100% sequence identity with any one of SEQ ID NO: 1-SEQ ID NO: 47, SEQ ID NO. 105, and SEQ ID NO: 107 can comprise reduced or substantially no nucleic acid cleavage activity.

In some embodiments, the N-terminal amino acid sequence of the programmable nuclease is not MISKMIKPTV (SEQ ID NO: 113). In some embodiments, the programmable nuclease does not include the amino acid sequence MISKMIKPTV (SEQ ID NO: 114).

In some embodiments, the N-terminal amino acid sequence of the programmable nuclease is not MISK (SEQ ID NO: 115). In some embodiments, the programmable nuclease does not include the amino acid sequence MISK (SEQ ID NO: 115).

In some embodiments, a composition comprises a first programmable nuclease described herein and a second programmable nuclease described herein. In some embodiments, a complex comprises a first programmable nuclease described herein and a second programmable nuclease described herein. In preferred embodiments, a complex comprises a first programmable nuclease described herein and a second programmable nuclease described herein, wherein the first and second programmable nucleases are the same programmable nuclease. In some embodiments, the first and second programmable nucleases form a dimer. In some preferred embodiments, the first and second programmable nucleases form a homodimer.

In some embodiments, a dimer comprises a first programmable nuclease described herein and a second programmable nuclease described herein. In preferred embodiments, the dimer is a homodimer wherein the first and second programmable nucleases are the same.

In some embodiments, a programmable nuclease may be a programmable nickase. The present disclosure provides compositions of programmable nickases, capable of introducing a break in a single strand of a double stranded DNA (dsDNA) (“nicking”). In some embodiments the programmable nickase is a programmable DNA nickase. Said programmable nickases can be coupled to a guide nucleic acid that targets a particular region of interest in the dsDNA. In some embodiments, two programmable nickases are combined and delivered together to generate two strand breaks. For example, a first programmable nickase can be targeted to and nicks a first region of dsDNA and a second programmable nickase can be targeted to and nicks a second region of the same dsDNA on the opposing strand. When combined and delivered together to generate nicks on opposing strands of the dsDNA, two strand breaks in the dsDNA can be generated. The strand breaks can be repaired and rejoined by non-homologous end joining (NHEJ) or homology directed repair (HDR). Thus, two programmable nickases disclosed herein can be combined to selectively edit nucleic acid sequences. This can be useful in any genome editing method, for example, used for therapeutic applications to treat a disease or disorder, or for agricultural applications.

In some embodiments, a programmable nuclease as disclosed herein can be used for genome editing purposes to generate strand breaks in order to excise a region of DNA or to subsequently introduce a region of DNA (e.g., donor DNA).

In some embodiments, the programmable nucleases (e.g., nickases) disclosed herein can be used in DNA Endonuclease Targeted CRISPR TransReporter (DETECTR) assays. In some embodiments, the programmable nuclease is a programmable nickase. A DETECTR assay can utilize the trans-cleavage abilities of some programmable nucleases to achieve fast and high-fidelity detection of a target nucleic acid in a sample. The target nucleic acid can be DNA or RNA. For example, following target DNA extraction from a biological sample, crRNA comprising a portion that is complementary to the target DNA of interest can bind to the target DNA sequence, initiating indiscriminate ssDNase activity by the programmable nuclease. In some embodiments, the extracted DNA is amplified by PCR or isothermal amplification reactions before contacting the DNA to the programmable nuclease complexed with a guide RNA. Upon hybridization with the target DNA, the trans-cleavage activity of the programmable nuclease is activated, which can then cleave an ssDNA fluorescence-quenching (FQ) reporter molecule. Cleavage of the reporter molecule can provide a fluorescent readout indicating the presence of the target DNA in the sample. In some embodiments, the programmable nucleases disclosed herein can be combined, or multiplexed, with other programmable nucleases in a DETECTR assay. The principles of the DETECTR assay are described in Chen et al. (Science 2018 April 27; 360(6387):436-439) and can be modified to facilitate the use of the programmable nucleases described herein. In some embodiments, the programmable nucleases disclosed herein can be used in a specific high-sensitivity enzymatic reporter unlocking (SHERLOCK) assay. The principles of the SHERLOCK assay are described in Kellner et al. (Nat Protoc. 2019 October; 14(10):2986-3012) and can be modified to facilitate the use of the programmable nucleases described herein. Thus some embodiments provide a method of detecting a target nucleic acid in a sample, the method comprising: contacting a sample comprising a target nucleic acid with (a) a programmable CasΦ nuclease disclosed herein, (b) a guide RNA comprising a region that binds to the programmable CasΦ nuclease and an additional region that binds to the target nucleic acid, and (c) a detector nucleic acid that does not bind the guide RNA; cleaving the detector nucleic acid by the programmable CasΦ nuclease; and detecting the target nucleic acid by measuring a signal produced by the cleavage of the detector nucleic acid. In preferred embodiments, the detector nucleic acid is a single stranded DNA reporter.

The programmable nucleases of the present disclosure can show enhanced activity, as measured by enhanced cleavage of an ssDNA-FQ reporter, under certain conditions in the presence of the target DNA. For example, the programmable nucleases of the present disclosure can have variable levels of activity based on a buffer formulation, a pH level, temperature, or salt. Buffers consistent with the present disclosure include phosphate buffers, Tris buffers, and HEPES buffers. Programmable nucleases of the present disclosure can show optimal activity in phosphate buffers, Tris buffers, and HEPES buffers.

Programmable nucleases can also exhibit varying levels of nickase or double-stranded cleavage activity at different pH levels. For example, enhanced cleavage can be observed between pH 7 and pH 9. In some embodiments, programmable nuclease of the present disclosure exhibit enhanced cleavage at about pH 7, about pH 7.1, about pH 7.2, about pH 7.3, about pH 7.4, about pH 7.5, about pH 7.6, about pH 7.7, about pH 7.8, about pH 7.9, about pH 8, about pH 8.1, about pH 8.2, about pH 8.3, about pH 8.4, about pH 8.5, about pH 8.6, about pH 8.7, about pH 8.8, about pH 8.9, about pH 9, from pH 7 to 7.5, from pH 7.5 to 8, from pH 8 to 8.5, from pH 8.5 to 9, or from pH 7 to 8.5.

In some embodiments, the programmable nucleases of the present disclosure exhibit enhanced cleavage of ssDNA-FQ reporters DNA at a temperature of 25° C. to 50° C. in the presence of target DNA. For example, the programmable nucleases of the present disclosure can exhibit enhanced cleavage of an ssDNA-FQ reporter at about 25° C., about 26° C., about 27° C., about 28° C., about 29° C., about 30° C., about 31° C., about 32° C., about 33° C., about 34° C., about 35° C., about 36° C., about 37° C., about 38° C., about 39° C., about 40° C., about 41° C., about 42° C., about 43° C., about 44° C., about 45° C., about 46° C., about 47° C., about 48° C., about 49° C., about 50° C., from 30° C. to 40° C., from 35° C. to 45° C., or from 35° C. to 40° C.

The programmable nucleases of the present disclosure may not be sensitive to salt concentrations in a sample in the presence of the target DNA. Advantageously, said programmable nucleases can be active and capable of cleaving ssDNA-FQ-reporter sequences under varying salt concentrations from 25 nM salt to 200 mM salt. Various salts are consistent with this property of the programmable nucleases disclosed herein, including NaCl or KCl. The programmable nucleases of the present disclosure can be active at salt concentrations of from 25 nM to 500 nM salt, from 500 nM to 1000 nM salt, from 1000 nM to 2000 nM salt, from 2000 nM to 3000 nM salt, from 3000 nM to 4000 nM salt, from 4000 nM to 5000 nM salt, from 5000 nM to 6000 nM salt, from 6000 nM to 7000 nM salt, from 7000 nM to 8000 nM salt, from 8000 nM to 9000 nM salt, from 9000 nM to 0.01 mM salt, from 0.01 mM to 0.05 mM salt, from 0.05 mM to 0.1 mM salt, from 0.1 mM to 10 mM salt, from 10 mM to 100 mM salt, or from 100 mM to 500 mM salt. Thus, the programmable nucleases of the present disclosure can exhibit cleavage activity independent of the salt concentration in a sample.

Programmable nucleases of the present disclosure can be capable of cleaving any ssDNA-FQ reporter, regardless of its sequence. The programmable nucleases provided herein can, thus, be capable of cleaving a universal ssDNA FQ reporter. In some embodiments, the programmable nucleases provided herein cleave homopolymer ssDNA-FQ reporter comprising 5 to 20 adenines, 5 to 20 thymines, 5 to 20 cytosines, or 5 to 20 guanines. Programmable nucleases of the present disclosure, thus, are capable of cleaving ssDNA-FQ reporters also cleaved by programmable nucleases, as disclosed elsewhere herein, allowing for facile multiplexing of multiple programmable nickases and programmable nucleases in a single assay having a single ssDNA-FQ reporter.

Programmable nucleases of the present disclosure can bind a wild type protospacer adjacent motif (PAM) or a mutant PAM in a target DNA. In some embodiments the programmable CasΦ nucleases of the present disclosure recognizes and bind a protospacer adjacent motif (PAM) of 5′-TBN-3′, where B is one or more of C, G, or, T. For example, programmable CasΦ nucleases of the present disclosure may recognizes and bind a protospacer adjacent motif (PAM) of 5′-TTTN-3′. As another example, programmable CasΦ nucleases of the present disclosure may recognizes and bind a protospacer adjacent motif (PAM) of 5′-TTN-3.′ In some embodiments, the PAM is 5′-TTTA-3′, 5′-GTTK-3′, 5′-VTTK-3′, 5′-VTTS-3′, 5′-TTTS-3′ or 5′-VTTN-3′, where K is G or T, V is A, C or G, and S is C or G. In some embodiments, the PAM is 5′-GTTB-3′, wherein B is C, G, or, T.

In some embodiments of the present disclosure, the programmable CasΦ nucleases recognize and bind a PAM of 5′-NTTN-3′.

In some embodiments, when the programmable CasΦ nuclease or a variant thereof comprises at least 70%, at least 75%, at least 80%, at least 85%, at least 90%, at least 92%, at least 95%, at least 97%, at least 99%, or 100% sequence identity with SEQ ID NO: 2, the programmable CasΦ nuclease or a variant recognizes a 5′-GTTK-3′ PAM. In some embodiments, when the programmable CasΦ nuclease or a variant thereof comprises at least 70%, at least 75%, at least 80%, at least 85%, at least 90%, at least 92%, at least 95%, at least 97%, at least 99%, or 100% sequence identity with SEQ ID NO: 2, the programmable CasΦ nuclease or a variant recognizes a 5′-NTTN-3′ PAM.

In some embodiments, when the programmable CasΦ nuclease or a variant thereof comprises at least 70%, at least 75%, at least 80%, at least 85%, at least 90%, at least 92%, at least 95%, at least 97%, at least 99%, or 100% sequence identity with SEQ ID NO: 4, the programmable CasΦ nuclease or a variant recognizes a 5′-VTTK-3′ PAM. In some embodiments, when the programmable CasΦ nuclease or a variant thereof comprises at least 70%, at least 75%, at least 80%, at least 85%, at least 90%, at least 92%, at least 95%, at least 97%, at least 99%, or 100% sequence identity with SEQ ID NO: 4, the programmable CasΦ nuclease or a variant recognizes a 5′-NTTN-3′ PAM.

In some embodiments, when the programmable CasΦ nuclease or a variant thereof comprises at least 70%, at least 75%, at least 80%, at least 85%, at least 90%, at least 92%, at least 95%, at least 97%, at least 99%, or 100% sequence identity with SEQ ID NO: 11, the programmable CasΦ nuclease or a variant recognizes a 5′-VTTS-3′ PAM. In some embodiments, when the programmable CasΦ nuclease or a variant thereof comprises at least 70%, at least 75%, at least 80%, at least 85%, at least 90%, at least 92%, at least 95%, at least 97%, at least 99%, or 100% sequence identity with SEQ ID NO: 11, the programmable CasΦ nuclease or a variant recognizes a 5′-NTTN-3′ PAM.

In some embodiments, when the programmable CasΦ nuclease or a variant thereof comprises at least 70%, at least 75%, at least 80%, at least 85%, at least 90%, at least 92%, at least 95%, at least 97%, at least 99%, or 100% sequence identity with SEQ ID NO: 12, the programmable CasΦ nuclease or a variant recognizes a 5′-TTTS-3′ PAM. In some embodiments, when the programmable CasΦ nuclease or a variant thereof comprises at least 70%, at least 75%, at least 80%, at least 85%, at least 90%, at least 92%, at least 95%, at least 97%, at least 99%, or 100% sequence identity with SEQ ID NO: 12, the programmable CasΦ nuclease or a variant recognizes a 5′-NTTN-3′ PAM.

In some embodiments, when the programmable CasΦ nuclease or a variant thereof comprises at least 70%, at least 75%, at least 80%, at least 85%, at least 90%, at least 92%, at least 95%, at least 97%, at least 99%, or 100% sequence identity with SEQ ID NO: 18, the programmable CasΦ nuclease or a variant recognizes a 5′-VTTN-3′ PAM.

In some embodiments, when the programmable CasΦ nuclease or a variant thereof comprises at least 70%, at least 75%, at least 80%, at least 85%, at least 90%, at least 92%, at least 95%, at least 97%, at least 99%, or 100% sequence identity with SEQ ID NO: 20, the programmable CasΦ nuclease or a variant recognizes a 5′-NTNN-3′ PAM.

In some embodiments, when the programmable CasΦ nuclease or a variant thereof comprises at least 70%, at least 75%, at least 80%, at least 85%, at least 90%, at least 92%, at least 95%, at least 97%, at least 99%, or 100% sequence identity with SEQ ID NO: 20, the programmable CasΦ nuclease or a variant recognizes a 5′-TTN-3′ PAM.

In some embodiments, when the programmable CasΦ nuclease or a variant thereof comprises at least 70%, at least 75%, at least 80%, at least 85%, at least 90%, at least 92%, at least 95%, at least 97%, at least 99%, or 100% sequence identity with SEQ ID NO: 26, the programmable CasΦ nuclease or a variant recognizes a 5′-NTTG-3′ PAM.

In some embodiments, when the programmable CasΦ nuclease or a variant thereof comprises at least 70%, at least 75%, at least 80%, at least 85%, at least 90%, at least 92%, at least 95%, at least 97%, at least 99%, or 100% sequence identity with SEQ ID NO: 32, the programmable CasΦ nuclease or a variant recognizes a 5′-GTTB-3′ PAM, wherein B is C, G, or N.

In some embodiments, when the programmable CasΦ nuclease or a variant thereof comprises at least 70%, at least 75%, at least 80%, at least 85%, at least 90%, at least 92%, at least 95%, at least 97%, at least 99%, or 100% sequence identity with SEQ ID NO: 42, the programmable CasΦ nuclease or a variant recognizes a 5′-GTTN-3′ PAM.

In some embodiments, when the programmable CasΦ nuclease or a variant thereof comprises at least 70%, at least 75%, at least 80%, at least 85%, at least 90%, at least 92%, at least 95%, at least 97%, at least 99%, or 100% sequence identity with SEQ ID NO: 41, the programmable CasΦ nuclease or a variant recognizes a 5′-NTTN-3′ PAM.

In some embodiments, when the programmable CasΦ nuclease or a variant thereof comprises at least 70%, at least 75%, at least 80%, at least 85%, at least 90%, at least 92%, at least 95%, at least 97%, at least 99%, or 100% sequence identity with SEQ ID NO: 24, the programmable CasΦ nuclease or a variant recognizes a 5′-NTNN-3′ PAM.

In some embodiments, when the programmable CasΦ nuclease or a variant thereof comprises at least 70%, at least 75%, at least 80%, at least 85%, at least 90%, at least 92%, at least 95%, at least 97%, at least 99%, or 100% sequence identity with SEQ ID NO: 25, the programmable CasΦ nuclease or a variant recognizes a 5′-NTNN-3′ PAM.

The programmable nucleases and other reagents (e.g., a guide nucleic acid) can be formulated in a buffer disclosed herein. A wide variety of buffered solutions are compatible with the methods, compositions, reagents, enzymes, and kits disclosed herein. Buffers are compatible with different programmable nucleases described herein. Any of the methods, compositions, reagents, enzymes, or kits disclosed herein may comprise a buffer. These buffers may be compatible with the other reagents, samples, and support mediums as described herein for detection of an ailment, such as a disease, cancer, or genetic disorder, or genetic information, such as for phenotyping, genotyping, or determining ancestry. A buffer, as described herein, can enhance the cis- or trans-cleavage rates of any of the programmable nucleases described herein. The buffer can increase the discrimination of the programmable nucleases for the target nucleic acid. The methods as described herein can be performed in the buffer.

In some embodiments, a buffer may comprise one or more of a buffering agent, a salt, a crowding agent, or a detergent, or any combination thereof. A buffer may comprise a reducing agent. A buffer may comprise a competitor. Exemplary buffering agents include HEPES, TRIS, MES, ADA, PIPES, ACES, MOPSO, BIS-TRIS propane, BES, MOPS, TES, DISO, Trizma, TRICINE, GLY-GLY, HEPPS, BICINE, TAPS, A MPD, A MPSO, CHES, CAPSO, AMP, CAPS, phosphate, citrate, acetate, imidazole, or any combination thereof. A buffering agent may be compatible with a programmable nuclease. A buffer compatible with a programmable nuclease may comprise a buffering agent at a concentration of from 1 mM to 200 mM. A buffer compatible with a programmable nuclease may comprise a buffering agent at a concentration of from 10 mM to 30 mM. A buffer compatible with a programmable nuclease may comprise a buffering agent at a concentration of about 20 mM. A composition (e.g., a composition comprising a programmable nuclease) may have a pH of from 2.5 to 3.5. A composition (e.g., a composition comprising a programmable nuclease) may have a pH of from 3 to 4. A composition (e.g., a composition comprising a programmable nuclease) may have a pH of from 3.5 to 4.5. A composition (e.g., a composition comprising a programmable nuclease) may have a pH of from 4 to 5. A composition (e.g., a composition comprising a programmable nuclease) may have a pH of from 4.5 to 5.5. A composition (e.g., a composition comprising a programmable nuclease) may have a pH of from 5 to 6. A composition (e.g., a composition comprising a programmable nuclease) may have a pH of from 5.5 to 6.5. A composition (e.g., a composition comprising a programmable nuclease) may have a pH of from 6 to 7. A composition (e.g., a composition comprising a programmable nuclease) may have a pH of from 6.5 to 7.5. A composition (e.g., a composition comprising a programmable nuclease) may have a pH of from 7 to 8. A composition (e.g., a composition comprising a programmable nuclease) may have a pH of from 7.5 to 8.5. A composition (e.g., a composition comprising a programmable nuclease) may have a pH of from 8 to 9. A composition (e.g., a composition comprising a programmable nuclease) may have a pH of from 8.5 to 9.5. A composition (e.g., a composition comprising a programmable nuclease) may have a pH of from 9 to 10. A composition (e.g., a composition comprising a programmable nuclease) may have a pH of from 9.5 to 10.5.

A buffer may comprise a salt. Exemplary salts include NaCl, KCl, magnesium acetate, potassium acetate, CaCl₂) and MgCl₂. A buffer may comprise potassium acetate, magnesium acetate, sodium chloride, magnesium chloride, or any combination thereof. A buffer compatible with a programmable nuclease may comprise a salt at a concentration of from 5 mM to 100 mM. A buffer compatible with a programmable nuclease may comprise a salt at a concentration of from 5 mM to 10 mM. In some embodiments, a buffer compatible with a programmable nuclease comprises a salt from 1 mM to 60 mM. In some embodiments, a buffer compatible with a programmable nuclease comprises a salt from 1 mM to 10 mM. In some embodiments, a buffer compatible with a programmable nuclease comprises a salt at about 105 mM. In some embodiments, a buffer compatible with a programmable nuclease comprises a salt at about 55 mM. In some embodiments, a buffer compatible with a programmable nuclease comprises a salt at about 7 mM. In some embodiments, a buffer compatible with a programmable nuclease comprises a salt, wherein the salt comprises potassium acetate and magnesium acetate. In some embodiments, a buffer compatible with a programmable nuclease comprises a salt, wherein the salt comprises sodium chloride and magnesium chloride. In some embodiments, a buffer compatible with a programmable nuclease comprises a salt, wherein the salt comprises potassium chloride and magnesium chloride.

A buffer may comprise a crowding agent. Exemplary crowding agents include glycerol and bovine serum albumin. A buffer may comprise glycerol. A crowding agent may reduce the volume of solvent available for other molecules in the solution, thereby increasing the effective concentrations of said molecules. A buffer compatible with a programmable nuclease may comprise a crowding agent at a concentration of from 0.01% (v/v) to 10% (v/v). A buffer compatible with a programmable nuclease may comprise a crowding agent at a concentration of from 0.5% (v/v) to 10% (v/v).

A buffer may comprise a detergent. Exemplary detergents include Tween, Triton-X, and IGEPAL. A buffer may comprise Tween, Triton-X, or any combination thereof. A buffer compatible with a programmable nuclease may comprise Triton-X. A buffer compatible with a programmable nuclease may comprise IGEPAL CA-630. In some embodiments, a buffer compatible with a programmable nuclease comprises a detergent at a concentration of 2% (v/v) or less. A buffer compatible with a programmable nuclease may comprise a detergent at a concentration of 2% (v/v) or less. A buffer compatible with a programmable nuclease may comprise a detergent at a concentration of from 0.00001% (v/v) to 0.01% (v/v). A buffer compatible with a programmable nuclease may comprise a detergent at a concentration of about 0.01% (v/v).

A buffer may comprise a reducing agent. Exemplary reducing agents comprise dithiothreitol (DTT), 8-mercaptoethanol (BME), or tris(2-carboxyethyl)phosphine (TCEP). A buffer compatible with a programmable nuclease may comprise DTT. A buffer compatible with a programmable nuclease may comprise a reducing agent at a concentration of from 0.01 mM to 100 mM. A buffer compatible with a programmable nuclease may comprise a reducing agent at a concentration of from 0.1 mM to 10 mM. A buffer compatible with a programmable nuclease may comprise a reducing agent at a concentration of from 0.5 mM to 2 mM. A buffer compatible with a programmable nuclease may comprise a reducing agent at a concentration of from 0.01 mM to 100 mM. A buffer compatible with a programmable nuclease may comprise a reducing agent at a concentration of from 0.1 mM to 10 mM. A buffer compatible with a programmable nuclease may comprise a reducing agent at a concentration of about 1 mM.

A buffer compatible with a programmable nuclease may comprise a competitor. Exemplary competitors compete with the target nucleic acid or the reporter nucleic acid for cleavage by the programmable nuclease. Exemplary competitors include heparin, and imidazole, and salmon sperm DNA. A buffer compatible with a programmable nuclease may comprise a competitor at a concentration of from 1 μg/mL to 100 μg/mL. A buffer compatible with a programmable nuclease may comprise a competitor at a concentration of from 40 μg/mL to 60 μg/mL.

In some embodiments, a programmable CasΦ nuclease is described as a “nickase” if the predominant cleavage product is a nicked nucleic acid when the target nucleic acid is a double-stranded nucleic acid. In some embodiments, a programmable CasΦ nuclease cleaves both strands of a double-stranded target nucleic acid. In some embodiments, the target nucleic acid is DNA. In some embodiments, the target nucleic acid is double-stranded DNA.

Where a programmable CasΦ nuclease disclosed herein cleaves both strands of a double-stranded target nucleic acid, the strand break may be a staggered cut with a 5′ overhang. In some embodiments, the 5′ overhang is an overhang of between 5 and 10 nucleotides. In some embodiments, the 5′ overhang is an overhang of 5 or 6 nucleotides. In some embodiments, the 5′ overhang is an overhang of 9 or 10 nucleotides.

In some embodiments, where the programmable CasΦ nuclease or a variant thereof comprises at least 70%, at least 75%, at least 80%, at least 85%, at least 90%, at least 92%, at least 95%, at least 97%, at least 99%, or 100% sequence identity with SEQ ID NO: 20, the 5′ overhang is a 9 or 10 nucleotide overhang. In preferred embodiments, where the programmable CasΦ nuclease or a variant thereof comprises at least 90% sequence identity with SEQ ID NO: 20, the 5′ overhang is a 9 or 10 nucleotide overhang. In further preferred embodiments, where the programmable CasΦ nuclease or a variant thereof comprises the amino acid sequence of SEQ ID NO: 20, the 5′ overhang is a 9 or 10 nucleotide overhang.

In some embodiments, where the programmable CasΦ nuclease or a variant thereof comprises at least 70%, at least 75%, at least 80%, at least 85%, at least 90%, at least 92%, at least 95%, at least 97%, at least 99%, or 100% sequence identity with SEQ ID NO: 22, the 5′ overhang is a 9 or 10 nucleotide overhang. In preferred embodiments, where the programmable CasΦ nuclease or a variant thereof comprises at least 90% sequence identity with SEQ ID NO: 22, the 5′ overhang is a 10 nucleotide overhang. In further preferred embodiments, where the programmable CasΦ nuclease or a variant thereof comprises the amino acid sequence of SEQ ID NO: 22, the 5′ overhang is a 10 nucleotide overhang.

In some embodiments, where the programmable CasΦ nuclease or a variant thereof comprises at least 70%, at least 75%, at least 80%, at least 85%, at least 90%, at least 92%, at least 95%, at least 97%, at least 99%, or 100% sequence identity with SEQ ID NO: 28, the 5′ overhang is a 9 nucleotide overhang. In preferred embodiments, where the programmable CasΦ nuclease or a variant thereof comprises at least 90% sequence identity with SEQ ID NO: 28, the 5′ overhang is a 9 nucleotide overhang. In further preferred embodiments, where the programmable CasΦ nuclease or a variant thereof comprises the amino acid sequence of SEQ ID NO: 28, the 5′ overhang is a 9 nucleotide overhang.

In some embodiments, where the programmable CasΦ nuclease or a variant thereof comprises at least 70%, at least 75%, at least 80%, at least 85%, at least 90%, at least 92%, at least 95%, at least 97%, at least 99%, or 100% sequence identity with SEQ ID NO: 40, the 5′ overhang is a 10 nucleotide overhang. In preferred embodiments, where the programmable CasΦ nuclease or a variant thereof comprises at least 90% sequence identity with SEQ ID NO: 40, the 5′ overhang is a 10 nucleotide overhang. In further embodiments, where the programmable CasΦ nuclease or a variant thereof comprises the amino acid sequence of SEQ ID NO: 40, the 5′ overhang is a 10 nucleotide overhang.

In some embodiments, where the programmable CasΦ nuclease or a variant thereof comprises at least 70%, at least 75%, at least 80%, at least 85%, at least 90%, at least 92%, at least 95%, at least 97%, at least 99%, or 100% sequence identity with SEQ ID NO: 37, the 5′ overhang is a 9 or 10 nucleotide overhang. In preferred embodiments, where the programmable CasΦ nuclease or a variant thereof comprises at least 90% sequence identity with SEQ ID NO: 37, the 5′ overhang is a 9 or 10 nucleotide overhang. In further preferred embodiments, where the programmable CasΦ nuclease or a variant thereof comprises the amino acid sequence of SEQ ID NO: 37, the 5′ overhang is a 9 or 10 nucleotide overhang.

In some embodiments, where the programmable CasΦ nuclease or a variant thereof comprises at least 70%, at least 75%, at least 80%, at least 85%, at least 90%, at least 92%, at least 95%, at least 97%, at least 99%, or 100% sequence identity with SEQ ID NO: 41, the 5′ overhang is a 9 or 10 nucleotide overhang. In preferred embodiments, where the programmable CasΦ nuclease or a variant thereof comprises at least 90% sequence identity with SEQ ID NO: 41, the 5′ overhang is a 9 or 10 nucleotide overhang. In further preferred embodiments, where the programmable CasΦ nuclease or a variant thereof comprises the amino acid sequence of SEQ ID NO: 41, the 5′ overhang is a 9 or 10 nucleotide overhang.

In some embodiments, where the programmable CasΦ nuclease or a variant thereof comprises at least 70%, at least 75%, at least 80%, at least 85%, at least 90%, at least 92%, at least 95%, at least 97%, at least 99%, or 100% sequence identity with SEQ ID NO: 12, the 5′ overhang is a 5 nucleotide overhang. In preferred embodiments, where the programmable CasΦ nuclease or a variant thereof comprises at least 90% sequence identity with SEQ ID NO: 12, the 5′ overhang is a 5 nucleotide overhang. In further preferred embodiments, where the programmable CasΦ nuclease or a variant thereof comprises the amino acid sequence of SEQ ID NO: 12, the 5′ overhang is a 5 nucleotide overhang.

In some embodiments, where the programmable CasΦ nuclease or a variant thereof comprises at least 70%, at least 75%, at least 80%, at least 85%, at least 90%, at least 92%, at least 95%, at least 97%, at least 99%, or 100% sequence identity with SEQ ID NO: 24, the 5′ overhang is a 6 nucleotide overhang. In preferred embodiments, where the programmable CasΦ nuclease or a variant thereof comprises at least 90% sequence identity with SEQ ID NO: 24, the 5′ overhang is a 6 nucleotide overhang. In further preferred embodiments, where the programmable CasΦ nuclease or a variant thereof comprises the amino acid sequence of SEQ ID NO: 24, the 5′ overhang is a 6 nucleotide overhang.

In some embodiments, where the programmable CasΦ nuclease or a variant thereof comprises at least 70%, at least 75%, at least 80%, at least 85%, at least 90%, at least 92%, at least 95%, at least 97%, at least 99%, or 100% sequence identity with SEQ ID NO: 25, the 5′ overhang is a 6 nucleotide overhang. In preferred embodiments, where the programmable CasΦ nuclease or a variant thereof comprises at least 90% sequence identity with SEQ ID NO: 25, the 5′ overhang is a 6 nucleotide overhang. In further preferred embodiments, where the programmable CasΦ nuclease or a variant thereof comprises the amino acid sequence of SEQ ID NO: 25, the 5′ overhang is a 6 nucleotide overhang.

In some embodiments, where the programmable CasΦ nuclease or a variant thereof comprises at least 70%, at least 75%, at least 80%, at least 85%, at least 90%, at least 92%, at least 95%, at least 97%, at least 99%, or 100% sequence identity with SEQ ID NO: 32, the 5′ overhang is a 6 nucleotide overhang. In preferred embodiments, where the programmable CasΦ nuclease or a variant thereof comprises at least 90% sequence identity with SEQ ID NO: 32, the 5′ overhang is a 6 nucleotide overhang. In further preferred embodiments, where the programmable CasΦ nuclease or a variant thereof comprises the amino acid sequence of SEQ ID NO: 32, the 5′ overhang is a 6 nucleotide overhang.

In some embodiments, where the programmable CasΦ nuclease or a variant thereof comprises at least 70%, at least 75%, at least 80%, at least 85%, at least 90%, at least 92%, at least 95%, at least 97%, at least 99%, or 100% sequence identity with SEQ ID NO: 33, the 5′ overhang is a 6 nucleotide overhang. In preferred embodiments, where the programmable CasΦ nuclease or a variant thereof comprises at least 90% sequence identity with SEQ ID NO: 33, the 5′ overhang is a 6 nucleotide overhang. In further preferred embodiments, where the programmable CasΦ nuclease or a variant thereof comprises the amino acid sequence of SEQ ID NO: 33, the 5′ overhang is a 6 nucleotide overhang.

In some embodiments, a programmable CasΦ nuclease rapidly cleaves a strand of a double-stranded target nucleic acid. In some embodiments, the programmable CasΦ nuclease cleaves the second strand of the target nucleic acid after it has cleaved the first strand of the target nucleic acid. The cleavage of target nucleic acid strands can be assessed in an in vitro cis-cleavage assay. To perform such as assay, the programmable CasΦ nuclease is complexed to its native crRNA, e.g. CasΦ.2 nuclease with the CasΦ.2 repeat, in buffer comprising 50 mM potassium acetate, 20 mM Tris-acetate, 10 mM magnesium acetate, 100 ug/ml BSA, and which is pH 7.9 at 25° C. The complexing is carried out for 20 minutes at room temperature, e.g. 20-22° C. The RNP is at a concentration of 200 nM. The target plasmid is a 2.2 kb super-coiled plasmid containing a target sequence, either 5′-TATTAAATACTCGTATTGCTGTTCGATTAT-3′ (SEQ ID NO: 116) or 5′-CACAGCTTGTCTGTAAGCGGATGCCATATG-3′ (SEQ ID NO: 117), which is immediately downstream of a 5′-GTTG-3′ or 5′-TTTG-3′ PAM. At time “0” 30 equal volumes of target plasmid, at 20 nM, and complexed RNP are mixed, so that the concentration of target plasmid is 10 nM and the concentration of complexed RNP is 100 nM. The incubation temperature is 37° C. The reaction is quenched at desired time points, e.g. 1, 3, 6, 15, 30 and 60 minutes, with reaction quench comprising 1 mg/ml proteinase K, 0.08% SDS and 15 mM EDTA. The sample incubates for 30 minutes at 37° C. to deproteinize. The cleavage is quantified by agarose gel analysis.

In some embodiments, a programmable CasΦ nuclease creates at least 50%, at least 55%, at least 60%, at least 65%, at least 70%, at least 75%, at least 80%, at least 85%, at least 90 or at least 95% of the maximum amount of nicked product within 1 minute, where the maximum amount of nicked product is the maximum amount detected within a 60 minute period from when the target plasmid is mixed with the programmable CasΦ nuclease. In preferred embodiments, at least 80% of the maximum amount of nicked product is created within 1 minute. In more preferred embodiments, at least 90% of the maximum amount of nicked product is created within 1 minute.

In some embodiments, a programmable CasΦ nuclease creates at least 50%, at least 55%, at least 60%, at least 65%, at least 70%, at least 75%, at least 80%, at least 85%, at least 90 or at least 95% of the maximum amount of linearized product is created within 1 minute, where the maximum amount of linearized product is the maximum amount detected within a 60 minute period from when the target plasmid is mixed with the programmable CasΦ nuclease. In preferred embodiments, at least 80% of the maximum amount of linearized product is created within 1 minute. In more preferred embodiments, at least 90% of the maximum amount of linearized product is created within 1 minute.

In some embodiments, a programmable CasΦ nuclease uses a co-factor. In some embodiments, the co-factor allows the programmable CasΦ nuclease to perform a function. In some embodiments, the function is pre-crRNA processing and/or target nucleic acid cleavage. As discussed in Jiang F. and Doudna J. A. (Annu. Rev. Biophys. 2017. 46:505-29), Cas9 uses divalent metal ions as co-factors. The suitability of a divalent metal ion as a cofactor can easily be assessed, such as by methods based on those described by Sundaresan et al. (Cell Rep. 2017 Dec. 26; 21(13): 3728-3739). In some embodiments, the co-factor is a divalent metal ion. In some embodiments, the divalent metal ion is selected from Mg²⁺, Mn²⁺, Zn²⁺, Ca²⁺, Cu²⁺. In a preferred embodiment, the divalent metal ion is Mg²⁺. In some embodiments, a programmable CasΦ nuclease forms a complex with a divalent metal ion. In preferred embodiments, a programmable CasΦ nuclease forms a complex with Mg²⁺.

In some aspects, the disclosure provides a composition comprising a programmable CasΦ nuclease disclosed herein and a cell, preferably wherein the cell is a eukaryotic cell. In some embodiments, a programmable CasΦ nuclease disclosed herein is in a cell, preferably wherein the cell is a eukaryotic cell.

In some aspects, the disclosure provides a composition comprising a nucleic acid encoding a programmable CasΦ nuclease disclosed herein and a cell, preferably wherein the cell is a eukaryotic cell. In some embodiments, a nucleic acid encoding a programmable CasΦ nuclease disclosed herein is in a cell, preferably wherein the cell is a eukaryotic cell.

### Guide Nucleic Acids

The methods and compositions of the disclosure may comprise a guide nucleic acid. The guide nucleic acid can bind to a target nucleic acid (e.g., a single strand of a target nucleic acid) or portion thereof. For example, the guide nucleic acid can bind to a target nucleic acid such as nucleic acid from a virus or a bacterium or other agents responsible for a disease, or an amplicon thereof, as described herein. The guide nucleic acid can bind to a target nucleic acid such as a nucleic acid from a bacterium, a virus, a parasite, a protozoa, a fungus or other agents responsible for a disease, or an amplicon thereof, as described herein. The target nucleic acid can comprise a mutation, such as a single nucleotide polymorphism (SNP). A mutation can confer for example, resistance to a treatment, such as antibiotic treatment. A mutation can confer a gene malfunction or gene knockout. A mutation can confer a disease, contribution to a disease, or risk for a disease, such as a liver disease or disorder, eye disease or disorder, cystic fibrosis, or muscle disease or disorder. The guide nucleic acid can bind to a target nucleic acid such as a nucleic acid, preferably DNA, from a cancer gene or gene associated with a genetic disorder, or an amplicon thereof, as described herein. The guide nucleic acid comprises a segment of nucleic acids that are reverse complementary to the target nucleic acid. Often the guide nucleic acid binds specifically to the target nucleic acid. The target nucleic acid may be a reversed transcribed RNA, DNA, DNA amplicon, or synthetic nucleic acids. The target nucleic acid can be a single-stranded DNA or DNA amplicon of a nucleic acid of interest. A guide nucleic acid may be a non-naturally occurring guide nucleic acid. A non-naturally occurring guide nucleic acid may comprise an engineered sequence having a repeat and a spacer that hybridizes to a target nucleic acid sequence of interest. A non-naturally occurring guide nucleic acid may be recombinantly expressed or chemically synthesized.

A guide nucleic acid (e.g. gRNA) may hybridize to a target sequence of a target nucleic acid. The guide nucleic acid can bind to a programmable nuclease.

In some embodiments, a gRNA comprises a crRNA. In some embodiments, a gRNA of a CasΦ polypeptide or variants thereof does not comprise a tracrRNA. As described by Jiang F. and Doudna J. A. (Annu. Rev. Biophys. 2017. 46:505-29), Cas9 cleavage activity requires a tracrRNA. A tracrRNA is a polynucleotide that hybridizes with a crRNA to allow crRNA maturation such that the crRNA can bind to the Cas nuclease and locate the Cas nuclease to a target sequence. In some embodiments, a programmable CasΦ nuclease disclosed herein does not require a tracrRNA to locate and/or cleave a target nucleic acid. A crRNA may comprise a repeat region. Specifically, the crRNA of the guide nucleic acid may comprise a repeat region and a spacer region. The repeat region refers to the sequence of the crRNA that binds to the programmable nuclease. The spacer region refers to the sequence of the crRNA that hybridizes to a sequence of the target nucleic acid. In some embodiments, the repeat region may comprise mutations or truncations with respect to the repeat sequences in pre-crRNA. The repeat sequence of the crRNA may interact with a programmable nuclease, allowing for the guide nucleic acid and the programmable nuclease to form a complex. This complex may be referred to as a ribonucleoprotein (RNP) complex. The crRNA may comprise a spacer sequence. The spacer sequence may hybridize to a target sequence of the target nucleic acid, where the target sequence is a segment of a target nucleic acid. The spacer sequences may be reverse complementary to the target sequence. In some cases, the spacer sequence may be sufficiently reverse complementary to a target sequence to allow for hybridization, however, may not necessarily be 100% reverse complementary.

In some embodiments, a programmable nuclease may cleave a precursor RNA (“pre-crRNA”) to produce (or “process”) a guide RNA (gRNA), also referred to as a “mature guide RNA.” A programmable nuclease that cleaves pre-crRNA to produce a mature guide RNA is said to have pre-crRNA processing activity.

Programmable nucleases disclosed herein may process the repeat sequence of a crRNA, where the repeat sequence is the region of the crRNA that binds to the programmable nuclease. For example, crRNA may be delivered to a mammalian cell, e.g. a HEK293T cell, wherein the crRNA includes a full length repeat region which is 36 nucleotides in length, along with a programmable nuclease. The programmable nuclease then cleaves the repeat region of the crRNA so that the mature crRNA comprises a shorter repeat region (e.g. 24 nucleotides in length). Accordingly, in some embodiments, programmable nucleases disclosed herein are capable of cleaving the repeat region of a crRNA. In preferred embodiments, programmable nucleases disclosed herein are capable of cleaving the repeat region of a crRNA in mammalian cells.

The guide nucleic acid can bind specifically to the target nucleic acid. A guide nucleic acid can comprise a sequence that is, at least in part, reverse complementary to the sequence of a target nucleic acid.

The guide nucleic acid may be a non-naturally occurring guide nucleic acid. A non-naturally occurring guide nucleic acid may comprise an engineered sequence having a repeat and a spacer that hybridizes to a target nucleic acid sequence of interest. A non-naturally occurring guide nucleic acid may be recombinantly expressed or chemically synthesized.

A guide nucleic acid can comprise RNA, DNA, or a combination thereof. The term “gRNA” refers to a guide nucleic acid comprising RNA. A gRNA may include nucleosides that are not ribonucleic. In some embodiments, all nucleosides in a gRNA are ribonucleic. In some embodiments, some of the nucleosides in a gRNA are not ribonucleic. In embodiments where nucleosides in a gRNA are not ribonucleic, non-ribonucleic nucleosides may be naturally-occurring or non-naturally-occurring nucleosides. In some embodiments, inter-nucleoside links are phosphodiester bonds. In some embodiments, the inter-nucleoside link between at least two nucleosides in a guide nucleic acid is not a phosphodiester bond. In some embodiments, the inter-nucleoside link between at least two nucleosides is a non-natural inter-nucleoside linkage. Non-natural inter-nucleoside linkages include phosphorous and non-phosphorous inter-nucleoside linkages. Phosphorous inter-nucleoside linkages include phosphorothioate linkages and thiophosphate linkages. An inter-nucleoside linkage may comprise a “C3 spacer”. C3 spacers are known to the skilled person as comprising a chain of three carbon atoms.

Guide nucleic acids may be modified to improve genome editing efficiency, increase stability, reduce off-target effects, and/or increase the affinity of the guide nucleic acid for a CasΦ polypeptide disclosed herein. Modifications may include non-natural nucleotides and/or non-natural linkages. In addition or alternatively, one or more sugar moieties of the guide nucleic acid may be modified. Such sugar moiety modifications may include 2′-O-methyl (2′OMe,), 2′-0-methyoxy-ethyl and 2′ fluoro. In some embodiments, editing efficiency, or genome editing efficiency, is determined by analyzing the frequency of indel mutations in a nucleic acid or gene knockout. In some embodiments, the use of a flow cytometer or next generation sequencing may be used to analyze cells for indel mutations or gene knockout. In other embodiments, off-target effects may be detected using a flow cytometer, next generation sequencing, or CIRCLE-seq.

In some preferred embodiments, first 3 nucleosides (or one of the first 3 nucleosides, or a combination of the first 3 nucleosides) from the 5′ end of the repeat region comprise a 2′-O-methyl modification and the linkages between the 3 nucleosides at the 3′ end of the spacer region comprise phosphorothioate linkages.

In some embodiments, the first nucleoside at the 5′ end of the repeat region comprises a 2′-O-methyl modification. In some embodiments, the first two nucleosides at the 5′ end of the repeat region comprise 2′-O-methyl modifications. In some embodiments, the first three nucleosides at the 5′ end of the repeat region comprise 2′-O-methyl modifications. In some embodiments, the last nucleoside at the 3′ end of the spacer region comprises a 2′-O-methyl modification. In some embodiments, the last two nucleosides at the 3′ end of the spacer region comprise 2′-O-methyl modifications. In some embodiments, the last three nucleosides at the 3′ end of the spacer region comprise 2′-O-methyl modifications.

In some embodiments, the first 3 nucleosides (or one of the first 3 nucleosides, or a combination of the first 3 nucleosides) from the 5′ end of the repeat region and the 3 nucleosides at the 3′ end of the spacer region comprise a 2′-O-methyl modification, and the linkages between the 3 nucleosides at the 3′ end of the spacer region comprise phosphorothioate linkages.

In some embodiments, the first 3 nucleosides (or one of the first 3 nucleosides, or a combination of the first 3 nucleosides) from the 5′ end of the repeat region and the 3 nucleosides at the 3′ end of the spacer region comprise a 2′ fluoro modification.

In some embodiments, the first nucleoside at the 5′ end of the repeat region comprises a 2′ fluoro modification. In some embodiments, the first two nucleosides at the 5′ end of the repeat region comprise 2′ fluoro modifications. In some embodiments, the first three nucleosides at the 5′ end of the repeat region comprise 2′ fluoro modifications. In some embodiments, the last nucleoside at the 3′ end of the spacer region comprises a 2′ fluoro modification. In some embodiments, the last two nucleosides at the 3′ end of the spacer region comprise 2′ fluoro modifications. In some embodiments, the last three nucleosides at the 3′ end of the spacer region comprise 2′ fluoro modifications. In preferred embodiments, the last three nucleosides at the 3′ end of the spacer region comprise 2′ fluoro modifications.

In preferred embodiments, the first two nucleosides at the 5′ end of the repeat region comprise 2′-O-methyl modifications, the first two nucleosides at the 5′ end of the repeat are linked by a phosphorothioate linkage, and the last three nucleosides at the 3′ end of the spacer region comprise 2′ fluoro modifications.

In some embodiments, the linkage between the two nucleosides at the 5′ end of the repeat region comprises a 3C spacer and the linkage between the two nucleosides at the 3′ end of the spacer region comprises a 3C spacer.

In some embodiments, the guide nucleic acid comprises ribonucleic nucleosides and deoxyribonucleic nucleosides. In some embodiments, the guide nucleic acid is a guide RNA wherein the first, eighth and ninth nucleosides from the 5′ end of the spacer region and the four nucleosides at the 3′ end of the spacer region are deoxyribonucleic nucleosides.

In some embodiments, the guide nucleic acid comprises a polyA tail. In some preferred embodiments, the guide nucleic acid comprises a polyA tail at the 3′ end of the spacer region.

In some embodiments, a plurality of modified guides (e.g., a combination of modified guides disclosed herein) are complexed with one or more programmable nucleases (e.g., one or more programmable nucleases disclosed herein). In some examples, one or more of the plurality of modified guides comprise any of the nucleoside modifications described herein. In some examples, one or more of the plurality of the modified guides comprise any length of repeat or spacer region described herein. In some examples, one or more of the plurality of the modified guides comprise a repeat spacer length described herein, and a nucleoside modification described herein. In some embodiments, one or more of the plurality of modified guides comprise a repeat sequence from about 15 to about 20 nucleotides in length. In some embodiments, one or more of the plurality of modified guides comprise a spacer sequence or region from about 15 to about 20 nucleotides in length.

TABLE 2 provides illustrative crRNA sequences for use with the compositions and methods of the disclosure. In some embodiments, the crRNA sequence comprises at least 70%, at least 80%, at least 90%, at least 92%, at least 95%, at least 97%, or at least 99%, or 100% sequence identity to any one of SEQ ID NO: 48-SEQ ID NO: 86, or a reverse complement thereof. In some embodiments, the crRNA sequence comprises at least 70%, at least 80%, at least 90%, at least 92%, at least 95%, at least 97%, at least 99%, or 100% sequence identity to SEQ ID NO: 49 or a reverse complement thereof. In some embodiments, the crRNA sequence comprises at least 70%, at least 80%, at least 90%, at least 92%, at least 95%, at least 97%, at least 99%, or 100% sequence identity to SEQ ID NO: 51 or a reverse complement thereof. In some embodiments, the crRNA sequence comprises at least 70%, at least 80%, at least 90%, at least 92%, at least 95%, at least 97%, at least 99%, or 100% sequence identity to SEQ ID NO: 52 or a reverse complement thereof. In some embodiments, the crRNA sequence comprises at least 70%, at least 80%, at least 90%, at least 92%, at least 95%, at least 97%, at least 99%, or 100% sequence identity to SEQ ID NO: 54 or a reverse complement thereof. In some embodiments, the crRNA sequence comprises at least 70%, at least 80%, at least 90%, at least 92%, at least 95%, at least 97%, at least 99%, or 100% sequence identity to SEQ ID NO: 57 or a reverse complement thereof.

TABLE 2
Illustrative crRNA sequences

| CasΦ | crRNA repeat sequence | SEQ ID. |
|---|---|---|
| ortholog | (shown as DNA), 5′-to-3′ | NO. |
| CasΦ.01 | GGAGAGATCTCAAACGATTGCTCGATTAGTCGAGAC | 48 |
| CasΦ.02 | GTCGGAACGCTCAACGATTGCCCCTCACGAGGGGAC | 49 |
| CasΦ.04 | ACCAAAACGACTATTGATTGCCCAGTACGCTGGGAC | 50 |
| CasΦ.07 | GGATCCAATCCTTTTTGATTGCCCAATTCGTTGGGAC | 51 |
| CasΦ.10 | GGATCTGAGGATCATTATTGCTCGTTACGACGAGAC | 52 |
| CasΦ.11 | CCTGCGAAACCTTTTGATTGCTCAGTACGCTGAGAC | 53 |
| CasΦ.12 | CTTTCAAGACTAATAGATTGCTCCTTACGAGGAGAC | 54 |
| CasΦ.13 | GTAGAAGACCTCGCTGATTGCTCGGTGCGCCGAGAC | 55 |
| CasΦ.17 | ATGGCAACAGACTCTCATTGCGCGGTACGCCGCGAC | 56 |
| CasΦ.18 | ACCAAAACGACTATTGATTGCCCAGTACGCTGGGAC | 57 |
| CasΦ.19 | GTCGCTCTCTAACGCTTGCCCAGTACGCTGGGAC | 58 |
| CasΦ.20 | GCTGGAAGACTCAATGATGGCTCCTTACGAGGAGAC | 59 |
| CasΦ.21 | GGTTGAACCCTCAACAGATTGCTCGGTAAGCCGAGAC | 60 |
| CasΦ.22 | GGTTGAACCCTCAACAGATTGCTCGGTAAGCCGAGAC | 61 |
| CasΦ.23 | CTTGAAATCCTGTCAGATTGCTCCCTTCGGGGAGAC | 62 |
| CasΦ.24 | GCTGGAAGACTCAATGATGGCTCCTTACGAGGAGAC | 63 |
| CasΦ.25 | GCTGGAAGACTCAATGATGGCTCCTTACGAGGAGAC | 64 |
| CasΦ.26 | CTAGGAACGCACGCAGATTGCTCGGTACGCCGAGAC | 65 |
| CasΦ.27 | ATTGCAACGCCTAAAGATTGCTCGATACGTCGAGAC | 66 |
| CasΦ.28 | GTTCGGCRAYCCTTTGATTGCTCAGTACGCTGAGAC | 67 |
| CasΦ.29 | GTTGAACCTAGATCAGATGGCTCAGTACGCTGAGAC | 68 |
| CasΦ.30 | CCCTCAACACGTCAGAAATGCCCGGCACGCCGGGAC | 69 |
| CasΦ.31 | GTCGCAAGACTCGAATAATTGCCCCTCTATGGGGAC | 70 |
| CasΦ.32 | GCTGGGGACCGATCCTGATTGCTCGCTGCGGCGAGAC | 71 |
| CasΦ.33 | CTCTCAATGGATAACGATTGCTCTCTACGGAGAGAC | 72 |
| CasΦ.34 | GCTGGAAGACTCAATGATGGCTCCTTACGAGGAGAC | 73 |
| CasΦ.35 | GTTGAACCCTCAACAGATTGCTCGGTAAGCCGAGAC | 74 |
| CasΦ.36 | GTCGCAAGACTCGAATAATTGCCCCTCTATGGGGAC | 75 |
| CasΦ.37 | GTCGGAACGCTCAACGATTGCCCCTCACGAGGGGAC | 76 |
| CasΦ.38 | GTTGAACCTAGATCAGATGGCTCAGTACGCTGAGAC | 77 |
| CasΦ.39 | CTCTCAATGGATAACGATTGCTCTCTACGGAGAGAC | 78 |
| CasΦ.41 | ACTGAAACCACCAACGATTGCGCTCCTCGGAGCGAC | 79 |
| CasΦ.42 | ACCAAAACGACTATTGATTGCCCAGTACGCTGGGAC | 80 |
| CasΦ.43 | GTTGAACCTAGATCAGATGGCTCAGTACGCTGAGAC | 81 |
| CasΦ.44 | GTTGAACCCTCAACAGATTGCTCGGTAAGCCGAGAC | 82 |
| CasΦ.45 | GTTGAACCTAGATCAGATGGCTCAGTACGCTGAGAC | 83 |
| CasΦ.46 | GTCGGAACGCTCAACGATTGCCCCTCACGAGGGGAC | 84 |
| CasΦ.47 | GGTTGAACCCTCAACAGATTGCTCGGTAAGCCGAGAC | 85 |
| CasΦ.48 | GGTTGAACCCTCAACAGATTGCTCGGTAAGCCGAGAC | 86 |

In some embodiments, the programmable nuclease disclosed herein is used in conjunction with a specific crRNA sequence. In some embodiments, the crRNA sequence comprises at least 70%, at least 80%, at least 90%, at least 92%, at least 95%, at least 97%, at least 99%, or 100% sequence identity to any one of SEQ ID NO: 48-SEQ ID NO: 86, or a reverse complement thereof. In some embodiments, the crRNA sequence comprises at least 70%, at least 80%, at least 90%, at least 92%, at least 95%, at least 97%, at least 99%, or 100% sequence identity to SEQ ID NO: 49 or a reverse complement thereof. In some embodiments, the crRNA sequence comprises at least 70%, at least 80%, at least 90%, at least 92%, at least 95%, at least 97%, at least 99%, or 100% sequence identity to SEQ ID NO: 51 or a reverse complement thereof. In some embodiments, the crRNA sequence comprises at least 70%, at least 80%, at least 90%, at least 92%, at least 95%, at least 97%, at least 99%, or 100% sequence identity to SEQ ID NO: 52 or a reverse complement thereof. In some embodiments, the crRNA sequence comprises at least 70%, at least 80%, at least 90%, at least 92%, at least 95%, at least 97%, at least 99%, or 100% sequence identity to SEQ ID NO: 54 or a reverse complement thereof. In some embodiments, the crRNA sequence comprises at least 70%, at least 80%, at least 90%, at least 92%, at least 95%, at least 97%, at least 99%, or 100% sequence identity to SEQ ID NO: 57 or a reverse complement thereof.

In some embodiments, the activity of a programmable CasΦ nuclease can be supported by a crRNA comprising any of the crRNA repeat sequences recited in TABLE 2. In some embodiments, the activity of a programmable CasΦ nuclease can be supported by a crRNA comprising a crRNA repeat sequence comprising at least 70%, at least 80%, at least 90%, at least 92%, at least 95%, at least 97%, at least 99%, or 100% sequence identity to any one of SEQ ID NO: 48-SEQ ID NO: 86.

In some embodiments, the crRNA repeat sequence comprises a hairpin. In some embodiments, the hairpin is in the 3′ portion of the crRNA repeat sequence. The hairpin comprises a double-stranded stem portion and a single-stranded loop portion. In preferred embodiments, one stand of the stem portion comprises a CYC sequence and the other strand comprises a GRG sequence, wherein Y and R are complementary. In preferred embodiments, the crRNA repeat comprises a GAC sequence at the 3′ end. In more preferred embodiments, the G of the GAC sequence is in the stem portion of the hairpin. In some embodiments, each strand of the stem portion comprises 3, 4, 5, 6, 7, 8, 9 or 10 nucleotides. In preferred embodiments, each strand of the stem portion comprises 3, 4 or 5 nucleotides. In some embodiments, the loop portion comprises 2, 3, 4, 5, 6, 7, 8, 9, 10 or more nucleotides. In preferred embodiments, the loop portion comprises 2, 3, 4, 5 or 6 nucleotides. In most preferred embodiments, the loop portion comprises 4 nucleotides. In some embodiments, the nucleotides are naturally occurring nucleotides. In some embodiments, the nucleotides are synthetic nucleotides.

In some cases, the guide nucleic acid is not naturally occurring and made by artificial combination of otherwise separate segments of sequence. Often, the artificial combination is performed by chemical synthesis, by genetic engineering techniques, or by the artificial manipulation of isolated segments of nucleic acids. In some cases, the segment of a guide nucleic acid that comprises a sequence that is reverse complementary to the target nucleic acid is 20 nucleotides in length. A guide nucleic acid can have at least 10, 11, 12, 13, 14, 15, 16, 17, 18, 19, 20, 21, 22, 23, 24, 25, 26, 27, 28, 29, or 30 nucleotides reverse complementary to a target nucleic acid. In some cases, the guide nucleic acid can be 10, 11, 12, 13, 14, 15, 16, 17, 18, 19, 20, 21, 22, 23, 24, 25, 26, 27, 28, 29, or 30 nucleotides in length. For example, a guide nucleic acid may be at least 10 bases. In some embodiments, a guide nucleic acid may be from 10 to 50 bases. In some embodiments, a guide nucleic acid may be at least 25 bases. In some cases, the guide nucleic acid has from exactly or about 12 nucleotides (nt) to about 80 nt, from about 12 nt to about 50 nt, from about 12 nt to about 45 nt, from about 12 nt to about 40 nt, from about 12 nt to about 35 nt, from about 12 nt to about 30 nt, from about 12 nt to about 25 nt, from about 12 nt to about 20 nt, from about 12 nt to about 19 nt, from about 19 nt to about 20 nt, from about 19 nt to about 25 nt, from about 19 nt to about 30 nt, from about 19 nt to about 35 nt, from about 19 nt to about 40 nt, from about 19 nt to about 45 nt, from about 19 nt to about 50 nt, from about 19 nt to about 60 nt, from about 20 nt to about 25 nt, from about 20 nt to about 30 nt, from about 20 nt to about 35 nt, from about 20 nt to about 40 nt, from about 20 nt to about 45 nt, from about 20 nt to about 50 nt, or from about 20 nt to about 60 nt reverse complementary to a target nucleic acid. In some cases, the guide nucleic acid has from about 10 nt to about 60 nt, from about 20 nt to about 50 nt, or from about 30 nt to about 40 nt reverse complementary to a target nucleic acid. It is understood that the sequence of a guide nucleic acid need not be 100% reverse complementary to that of its target nucleic acid to be specifically hybridizable, hybridizable, or bind specifically. The guide nucleic acid can have a sequence comprising at least one uracil in a region from nucleic acid residue 5 to 20 that is reverse complementary to a modification variable region in the target nucleic acid. The guide nucleic acid, in some cases, has a sequence comprising at least one uracil in a region from nucleic acid residue 5 to 9, 10 to 14, or 15 to 20 that is reverse complementary to a modification variable region in the target nucleic acid. The guide nucleic acid can have a sequence comprising at least one uracil in a region from nucleic acid residue 5 to 20 that is reverse complementary to a methylation variable region in the target nucleic acid. The guide nucleic acid, in some cases, has a sequence comprising at least one uracil in a region from nucleic acid residue 5 to 9, 10 to 14, or 15 to 20 that is reverse complementary to a methylation variable region in the target nucleic acid. The guide nucleic acid can hybridize with a target nucleic acid.

In some instances, compositions comprise shorter versions of the guide nucleic acids disclosed herein. For instance, the guide nucleic acid sequence may consist of a portion of a guide nucleic acid disclosed herein. In some instances, shorter versions may provide enhanced activity relative to their longer versions. Examples of longer versions and shorter versions of guide RNA for CasΦ.12 are shown in Tables I, K, M, O, Q, S, U, and W, and Tables AB-AF, respectively, wherein the shorter versions are produced by removing sixteen nucleotides from the 5′ end of the long version and three nucleotides from the 3′ end of the long version. In some instances, the long version is a CasΦ.32 guide nucleic acid described in Tables J, L, N, P, R, T, V, X, and the short version is a guide nucleic acid without the sixteen nucleotides at the 5′ end of the long version and without the three nucleotides at the 3′ end of the long version.

The guide nucleic acid (e.g., a non-naturally occurring guide nucleic acid) can be selected from a group of guide nucleic acids that have been tiled against the nucleic acid sequence of a strain of an infection or genomic locus of interest. The guide nucleic acid can be selected from a group of guide nucleic acids that have been tiled against the nucleic acid sequence of a target nucleic acid, for example, a strain of HPV16 or HPV18. Often, guide nucleic acids that are tiled against the nucleic acid of a strain of an infection or genomic locus of interest can be pooled for use in a method described herein. Often, these guide nucleic acids are pooled for detecting a target nucleic acid in a single assay. The pooling of guide nucleic acids that are tiled against a single target nucleic acid can enhance the detection of the target nucleic using the methods described herein. The pooling of guide nucleic acids that are tiled against a single target nucleic acid can ensure broad coverage of the target nucleic acid within a single reaction using the methods described herein. The tiling, for example, is sequential along the target nucleic acid. Sometimes, the tiling is overlapping along the target nucleic acid. In some instances, the tiling comprises gaps between the tiled guide nucleic acids along the target nucleic acid. In some instances, the tiling of the guide nucleic acids is non-sequential. Often, a method for detecting a target nucleic acid comprises contacting a target nucleic acid to a pool of guide nucleic acids and a programmable nuclease or nickase as disclosed herein, wherein a guide nucleic acid sequence of the pool of guide nucleic acids has a sequence selected from a group of tiled guide nucleic acid that correspond to nucleic acid sequence of a target nucleic acid; and assaying for a signal produce by cleavage of at least some nucleic acids of a reporter of a population of nucleic acids of a reporter. Pooling of guide nucleic acids can ensure broad spectrum identification, or broad coverage, of a target species within a single reaction. This can be particularly helpful in diseases or indications, like sepsis, that may be caused by multiple organisms.

In some embodiments, the spacer sequence is between 10 and 35 nucleotides in length, between 10 and 30 nucleotides in length, between 15 and 30 nucleotides in length, between 10 and 25 nucleotides in length, between 15 and 25 nucleotides in length, between 17 and 30 nucleotides in length, between 17 and 25 nucleotides in length, between 17 and 22 nucleotides in length, or between 17 and 20 nucleotides in length. In preferred embodiments, the spacer sequence between 17 and 25 nucleotides in length. In more preferred embodiments, the spacer sequence is between 17 and 20 nucleotides in length. In most preferred embodiments, the spacer sequence is 17 nucleotides in length.

In some embodiments, the repeat sequence is between 15 and 40 nucleotides in length, between 15 and 36 nucleotides in length, between 18 and 36 nucleotides in length, between 18 and 30 nucleotides in length, between 18 and 25 nucleotides in length, between 18 and 22 nucleotides in length, between 18 and 20 nucleotides in length. In preferred embodiments, the repeat sequence is between 20 and 22 nucleotides in length. In more preferred embodiments, the repeat sequence is 20 nucleotides in length.

The spacer region of guide nucleic acids for CasΦ polypeptides disclosed herein comprise a seed region. In some embodiments, the seed regions do not tolerate mismatches in the complentarity of a spacer and a target sequence within about 1 to about 20 nucleotides from the 5′ end of a spacer sequence. The seed region starts from the 5′ end of the spacer sequence and is a region in which mismatches in the complementarity between the spacer sequence and the target sequence are not tolerated when the guide nucleic acid is bound to a CasΦ polypeptide such that the guide nucleic acid does not hybridize to the target sequence to allow cleavage of the target nucleic acid by the CasΦ polypeptide. In some embodiments, the seed region comprises between 10 and 20 nucleosides, between 12 and 20 nucleosides, between 14 and 20 nucleosides, between 14 and 18 nucleosides, between 10 and 16 nucleosides, between 12 and 16 nucleosides, or between 14 and 16 nucleosides. In preferred embodiments, the seed region comprises 16 nucleotides.

A programmable nuclease of the present disclosure may be activated to exhibit cleavage activity (e.g., cis-cleavage of a target nucleic acid or trans-cleavage of a collateral nucleic acid) upon binding of a ribonucleoprotein (RNP) complex to a target nucleic acid, in which the spacer of the crRNA of the gRNA hybridizes to the target nucleic acid.

TABLE A
spacer sequences of gRNAs targeting human TRAC in T cells

| | Spacer sequence | | |
|---|---|---|---|
| Name | (5′→3′), shown as DNA | Target | SEQ ID NO |
| R3040 | TGGATATCTGTGGGACAAGA | TRAC | 118 |
| R3041 | TCCCACAGATATCCAGAACC | TRAC | 119 |
| R3042 | GAGTCTCTCAGCTGGTACAC | TRAC | 120 |
| R3043 | AGAGTCTCTCAGCTGGTACA | TRAC | 121 |
| R3044 | TCACTGGATTTAGAGTCTCT | TRAC | 122 |
| R3045 | AGAATCAAAATCGGTGAATA | TRAC | 123 |
| R3046 | GAGAATCAAAATCGGTGAAT | TRAC | 124 |
| R3047 | ACCGATTTTGATTCTCAAAC | TRAC | 125 |
| R3048 | TTTGAGAATCAAAATCGGTG | TRAC | 126 |
| R3049 | GTTTGAGAATCAAAATCGGT | TRAC | 127 |
| R3050 | TGATTCTCAAACAAATGTGT | TRAC | 128 |
| R3051 | GATTCTCAAACAAATGTGTC | TRAC | 129 |
| R3052 | ATTCTCAAACAAATGTGTCA | TRAC | 130 |
| R3053 | TGACACATTTGTTTGAGAAT | TRAC | 131 |
| R3054 | TCAAACAAATGTGTCACAAA | TRAC | 132 |
| R3055 | GTGACACATTTGTTTGAGAA | TRAC | 133 |
| R3056 | CTTTGTGACACATTTGTTTG | TRAC | 134 |
| R3057 | TGATGTGTATATCACAGACA | TRAC | 135 |
| R3058 | TCTGTGATATACACATCAGA | TRAC | 136 |
| R3059 | GTCTGTGATATACACATCAG | TRAC | 137 |
| R3060 | TGTCTGTGATATACACATCA | TRAC | 138 |
| R3061 | AAGTCCATAGACCTCATGTC | TRAC | 139 |
| R3062 | CTCTTGAAGTCCATAGACCT | TRAC | 140 |
| R3063 | AAGAGCAACAGTGCTGTGGC | TRAC | 141 |
| R3064 | CTCCAGGCCACAGCACTGTT | TRAC | 142 |
| R3065 | TTGCTCCAGGCCACAGCACT | TRAC | 143 |
| R3066 | GTTGCTCCAGGCCACAGCAC | TRAC | 144 |
| R3067 | CACATGCAAAGTCAGATTTG | TRAC | 145 |
| R3068 | GCACATGCAAAGTCAGATTT | TRAC | 146 |
| R3069 | GCATGTGCAAACGCCTTCAA | TRAC | 147 |
| R3070 | AAGGCGTTTGCACATGCAAA | TRAC | 148 |
| R3071 | CATGTGCAAACGCCTTCAAC | TRAC | 149 |
| R3072 | TTGAAGGCGTTTGCACATGC | TRAC | 150 |
| R3073 | AACAACAGCATTATTCCAGA | TRAC | 151 |
| R3074 | TGGAATAATGCTGTTGTTGA | TRAC | 152 |
| R3075 | TTCCAGAAGACACCTTCTTC | TRAC | 153 |
| R3076 | CAGAAGACACCTTCTTCCCC | TRAC | 154 |
| R3077 | CCTGGGCTGGGGAAGAAGGT | TRAC | 155 |
| R3078 | TTCCCCAGCCCAGGTAAGGG | TRAC | 156 |
| R3079 | CCCAGCCCAGGTAAGGGCAG | TRAC | 157 |
| R3080 | TAAAAGGAAAAACAGACATT | TRAC | 158 |
| R3081 | CTAAAAGGAAAAACAGACAT | TRAC | 159 |
| R3082 | TTCCTTTTAGAAAGTTCCTG | TRAC | 160 |
| R3083 | TCCTTTTAGAAAGTTCCTGT | TRAC | 161 |
| R3084 | CCTTTTAGAAAGTTCCTGTG | TRAC | 162 |
| R3085 | CTTTTAGAAAGTTCCTGTGA | TRAC | 163 |
| R3086 | TAGAAAGTTCCTGTGATGTC | TRAC | 164 |
| R3136 | AGAAAGTTCCTGTGATGTCA | TRAC | 165 |
| R3137 | GAAAGTTCCTGTGATGTCAA | TRAC | 166 |
| R3138 | ACATCACAGGAACTTTCTAA | TRAC | 167 |
| R3139 | CTGTGATGTCAAGCTGGTCG | TRAC | 168 |
| R3140 | TCGACCAGCTTGACATCACA | TRAC | 169 |
| R3141 | CTCGACCAGCTTGACATCAC | TRAC | 170 |
| R3142 | TCTCGACCAGCTTGACATCA | TRAC | 171 |
| R3143 | AAAGCTTTTCTCGACCAGCT | TRAC | 172 |
| R3144 | CAAAGCTTTTCTCGACCAGC | TRAC | 173 |
| R3145 | CCTGTTTCAAAGCTTTTCTC | TRAC | 174 |
| R3146 | GAAACAGGTAAGACAGGGGT | TRAC | 175 |
| R3147 | AAACAGGTAAGACAGGGGTC | TRAC | 176 |

TABLE B
spacer sequences of gRNAs targeting human B2M in T cells

| | Spacer Sequence | | |
|---|---|---|---|
| Name | (5′→3′), shown as DNA | Target | SEQ ID NO |
| R3087 | AATATAAGTGGAGGCGTCGC | B2M | 177 |
| R3088 | ATATAAGTGGAGGCGTCGCG | B2M | 178 |
| R3089 | AGGAATGCCCGCCAGCGCGA | B2M | 179 |
| R3090 | CTGAAGCTGACAGCATTCGG | B2M | 180 |
| R3091 | GGGCCGAGATGTCTCGCTCC | B2M | 181 |
| R3092 | GCTGTGCTCGCGCTACTCTC | B2M | 182 |
| R3093 | CTGGCCTGGAGGCTATCCAG | B2M | 183 |
| R3094 | TGGCCTGGAGGCTATCCAGC | B2M | 184 |
| R3095 | ATGTGTCTTTTCCCGATATT | B2M | 185 |
| R3096 | TCCCGATATTCCTCAGGTAC | B2M | 186 |
| R3097 | CCCGATATTCCTCAGGTACT | B2M | 187 |
| R3098 | CCGATATTCCTCAGGTACTC | B2M | 188 |
| R3099 | GAGTACCTGAGGAATATCGG | B2M | 189 |
| R3100 | GGAGTACCTGAGGAATATCG | B2M | 190 |
| R3101 | CTCAGGTACTCCAAAGATTC | B2M | 191 |
| R3102 | AGGTTTACTCACGTCATCCA | B2M | 192 |
| R3103 | ACTCACGTCATCCAGCAGAG | B2M | 193 |
| R3104 | CTCACGTCATCCAGCAGAGA | B2M | 194 |
| R3105 | TCTGCTGGATGACGTGAGTA | B2M | 195 |
| R3106 | CATTCTCTGCTGGATGACGT | B2M | 196 |
| R3107 | CCATTCTCTGCTGGATGACG | B2M | 197 |
| R3108 | ACTTTCCATTCTCTGCTGGA | B2M | 198 |
| R3109 | GACTTTCCATTCTCTGCTGG | B2M | 199 |
| R3110 | AGGAAATTTGACTTTCCATT | B2M | 200 |
| R3111 | CCTGAATTGCTATGTGTCTG | B2M | 201 |
| R3112 | CTGAATTGCTATGTGTCTGG | B2M | 202 |
| R3113 | CTATGTGTCTGGGTTTCATC | B2M | 203 |
| R3114 | AATGTCGGATGGATGAAACC | B2M | 204 |
| R3115 | CATCCATCCGACATTGAAGT | B2M | 205 |
| R3116 | ATCCATCCGACATTGAAGTT | B2M | 206 |
| R3117 | AGTAAGTCAACTTCAATGTC | B2M | 207 |
| R3118 | TTCAGTAAGTCAACTTCAAT | B2M | 208 |
| R3119 | AAGTTGACTTACTGAAGAAT | B2M | 209 |
| R3120 | ACTTACTGAAGAATGGAGAG | B2M | 210 |
| R3121 | TCTCTCCATTCTTCAGTAAG | B2M | 211 |
| R3122 | CTGAAGAATGGAGAGAGAAT | B2M | 212 |
| R3123 | AATTCTCTCTCCATTCTTCA | B2M | 213 |
| R3124 | CAATTCTCTCTCCATTCTTC | B2M | 214 |
| R3125 | TCAATTCTCTCTCCATTCTT | B2M | 215 |
| R3126 | TTCAATTCTCTCTCCATTCT | B2M | 216 |
| R3127 | AAAAAGTGGAGCATTCAGAC | B2M | 217 |
| R3128 | CTGAAAGACAAGTCTGAATG | B2M | 218 |
| R3129 | AGACTTGTCTTTCAGCAAGG | B2M | 219 |
| R3130 | TCTTTCAGCAAGGACTGGTC | B2M | 220 |
| R3131 | CAGCAAGGACTGGTCTTTCT | B2M | 221 |
| R3132 | AGCAAGGACTGGTCTTTCTA | B2M | 222 |
| R3133 | CTATCTCTTGTACTACACTG | B2M | 223 |
| R3134 | TATCTCTTGTACTACACTGA | B2M | 224 |
| R3135 | AGTGTAGTACAAGAGATAGA | B2M | 225 |
| R3148 | TACTACACTGAATTCACCCC | B2M | 226 |
| R3149 | AGTGGGGGTGAATTCAGTGT | B2M | 227 |
| R3150 | CAGTGGGGGTGAATTCAGTG | B2M | 228 |
| R3151 | TCAGTGGGGGTGAATTCAGT | B2M | 229 |
| R3152 | TTCAGTGGGGGTGAATTCAG | B2M | 230 |
| R3153 | ACCCCCACTGAAAAAGATGA | B2M | 231 |
| R3154 | ACACGGCAGGCATACTCATC | B2M | 232 |
| R3155 | GGCTGTGACAAAGTCACATG | B2M | 233 |
| R3156 | GTCACAGCCCAAGATAGTTA | B2M | 234 |
| R3157 | TCACAGCCCAAGATAGTTAA | B2M | 235 |
| R3158 | ACTATCTTGGGCTGTGACAA | B2M | 236 |
| R3159 | CCCCACTTAACTATCTTGGG | B2M | 237 |

TABLE C
spacer sequences of gRNAs that target human PD1 in T cells

| Name | Spacer sequence (5′→3′) | Target | SEQ ID NO |
|---|---|---|---|
| R2921 | CCUUCCGCUCACCUCCGCCU | PD1 | 238 |
| R2922 | CCUUCCGCUCACCUCCGCCU | PD1 | 239 |
| R2923 | CGCUCACCUCCGCCUGAGCA | PD1 | 240 |
| R2924 | UCCACUGCUCAGGCGGAGGU | PD1 | 241 |
| R2925 | UAGCACCGCCCAGACGACUG | PD1 | 242 |
| R2926 | AGGCAUGCAGAUCCCACAGG | PD1 | 243 |
| R2927 | CACAGGCGCCCUGGCCAGUC | PD1 | 244 |
| R2928 | UCUGGGCGGUGCUACAACUG | PD1 | 245 |
| R2929 | GCAUGCCUGGAGCAGCCCCA | PD1 | 246 |
| R2930 | UAGCACCGCCCAGACGACUG | PD1 | 247 |
| R2931 | UGGCCGCCAGCCCAGUUGUA | PD1 | 248 |
| R2932 | CUUCCGCUCACCUCCGCCUG | PD1 | 249 |
| R2933 | CAGGGCCUGUCUGGGGAGUC | PD1 | 250 |
| R2934 | UCCCCAGCCCUGCUCGUGGU | PD1 | 251 |
| R2935 | GGUCACCACGAGCAGGGCUG | PD1 | 252 |
| R2936 | UCCCCUUCGGUCACCACGAG | PD1 | 253 |
| R2937 | GAGAAGCUGCAGGUGAAGGU | PD1 | 254 |
| R2938 | ACCUGCAGCUUCUCCAACAC | PD1 | 255 |
| R2939 | UCCAACACAUCGGAGAGCUU | PD1 | 256 |
| R2940 | GCACGAAGCUCUCCGAUGUG | PD1 | 257 |
| R2941 | AGCACGAAGCUCUCCGAUGU | PD1 | 258 |
| R2942 | GUGCUAAACUGGUACCGCAU | PD1 | 259 |
| R2943 | CUGGGGCUCAUGCGGUACCA | PD1 | 260 |
| R2944 | UCCGUCUGGUUGCUGGGGCU | PD1 | 261 |
| R2945 | CCCGAGGACCGCAGCCAGCC | PD1 | 262 |
| R2946 | UGUGACACGGAAGCGGCAGU | PD1 | 263 |
| R2947 | CGUGUCACACAACUGCCCAA | PD1 | 264 |
| R2948 | GGCAGUUGUGUGACACGGAA | PD1 | 265 |
| R2949 | CACAUGAGCGUGGUCAGGGC | PD1 | 266 |
| R2950 | CGCCGGGCCCUGACCACGCU | PD1 | 267 |
| R2951 | GGGGCCAGGGAGAUGGCCCC | PD1 | 268 |
| R2952 | AUCUGCGCCUUGGGGGCCAG | PD1 | 269 |
| R2953 | GAUCUGCGCCUUGGGGGCCA | PD1 | 270 |
| R2954 | CCAGACAGGCCCUGGAACCC | PD1 | 271 |
| R2955 | CCAGCCCUGCUCGUGGUGAC | PD1 | 272 |
| R2956 | UCUCUGGAAGGGCACAAAGG | PD1 | 273 |
| R2957 | GUGCCCUUCCAGAGAGAAGG | PD1 | 274 |
| R2958 | UGCCCUUCCAGAGAGAAGGG | PD1 | 275 |
| R2959 | UGCCCUUCUCUCUGGAAGGG | PD1 | 276 |
| R2960 | CAGAGAGAAGGGCAGAAGUG | PD1 | 277 |
| R2961 | GAACUGGCCGGCUGGCCUGG | PD1 | 278 |
| R2962 | GGAACUGGCCGGCUGGCCUG | PD1 | 279 |
| R2963 | CAAACCCUGGUGGUUGGUGU | PD1 | 280 |
| R2964 | GUGUCGUGGGCGGCCUGCUG | PD1 | 281 |
| R2965 | CCUCGUGCGGCCCGGGAGCA | PD1 | 282 |
| R2966 | UCCCUGCAGAGAAACACACU | PD1 | 283 |
| R2967 | CUCUGCAGGGACAAUAGGAG | PD1 | 284 |
| R2968 | UCUGCAGGGACAAUAGGAGC | PD1 | 285 |
| R2969 | CUCCUCAAAGAAGGAGGACC | PD1 | 286 |
| R2970 | UCCUCAAAGAAGGAGGACCC | PD1 | 287 |
| R2971 | UCUGUGGACUAUGGGGAGCU | PD1 | 288 |
| R2972 | UCUCGCCACUGGAAAUCCAG | PD1 | 289 |
| R2973 | CCAGUGGCGAGAGAAGACCC | PD1 | 290 |
| R2974 | CAGUGGCGAGAGAAGACCCC | PD1 | 291 |
| R2975 | CGCUAGGAAAGACAAUGGUG | PD1 | 292 |
| R2976 | UCUUUCCUAGCGGAAUGGGC | PD1 | 293 |
| R2977 | CCUAGCGGAAUGGGCACCUC | PD1 | 294 |
| R2978 | CUAGCGGAAUGGGCACCUCA | PD1 | 295 |
| R2979 | GCCCCUCUGACCGGCUUCCU | PD1 | 296 |
| R2980 | CUUGGCCACCAGUGUUCUGC | PD1 | 297 |
| R2981 | GCCACCAGUGUUCUGCAGAC | PD1 | 298 |
| R2982 | UGCAGACCCUCCACCAUGAG | PD1 | 299 |
| R2983 | UCCUGAGGAAAUGCGCUGAC | PD1 | 300 |
| R2984 | CCUCAGGAGAAGCAGGCAGG | PD1 | 301 |
| R2985 | CUCAGGAGAAGCAGGCAGGG | PD1 | 302 |
| R2986 | CAGGCCGUCCAGGGGCUGAG | PD1 | 303 |
| R2987 | AGACAUGAGUCCUGUGGUGG | PD1 | 304 |
| R2988 | AGGUCCUGCCAGCACAGAGC | PD1 | 305 |
| R2989 | AGGGAGCUGGACGCAGGCAG | PD1 | 306 |
| R2990 | AGCCCCGGGCCGCAGGCAGC | PD1 | 307 |
| R2991 | AGGCAGGAGGCUCCGGGGCG | PD1 | 308 |
| R2992 | GGGGCUGGUUGGAGAUGGCC | PD1 | 309 |
| R2993 | GAGAUGGCCUUGGAGCAGCC | PD1 | 310 |
| R2994 | GCUGCUCCAAGGCCAUCUCC | PD1 | 311 |
| R2995 | GAGCAGCCAAGGUGCCCCUG | PD1 | 312 |
| R2996 | GGGAUGCCACUGCCAGGGGC | PD1 | 313 |
| R2997 | CGGGAUGCCACUGCCAGGGG | PD1 | 314 |
| R2998 | GGCCCUGCGUCCAGGGCGUU | PD1 | 315 |
| R2999 | UCUGCUCCCUGCAGGCCUAG | PD1 | 316 |
| R3000 | UCUAGGCCUGCAGGGAGCAG | PD1 | 317 |
| R3001 | CCUGAAACUUCUCUAGGCCU | PD1 | 318 |
| R3002 | UGACCUUCCCUGAAACUUCU | PD1 | 319 |
| R3003 | CAGGGAAGGUCAGAAGAGCU | PD1 | 320 |
| R3004 | AGGGAAGGUCAGAAGAGCUC | PD1 | 321 |
| R3005 | CUGCCCUGCCCACCACAGCC | PD1 | 322 |
| R3006 | CCUGCCCUGCCCACCACAGC | PD1 | 323 |
| R3007 | ACACAUGCCCAGGCAGCACC | PD1 | 324 |
| R3008 | CACAUGCCCAGGCAGCACCU | PD1 | 325 |
| R3009 | CCUGCCCCACAAAGGGCCUG | PD1 | 326 |
| R3010 | GUGGGGCAGGGAAGCUGAGG | PD1 | 327 |
| R3011 | UGGGGCAGGGAAGCUGAGGC | PD1 | 328 |
| R3012 | CUGCCUCAGCUUCCCUGCCC | PD1 | 329 |
| R3013 | CAGGCCCAGCCAGCACUCUG | PD1 | 330 |
| R3014 | AGGCCCAGCCAGCACUCUGG | PD1 | 331 |
| R3015 | CACCCCAGCCCCUCACACCA | PD1 | 332 |
| R3016 | GGACCGUAGGAUGUCCCUCU | PD1 | 333 |

TABLE D
spacer sequences of gRNAs targeting human CIITA

| | Spacer sequence | | |
|---|---|---|---|
| Name | (5′→3′), shown as DNA | Target | SEQ ID NO |
| R4503 C2TA_T1.1 | CTACACAATGCGTTGCCTGG | CIITA | 334 |
| R4504 C2TA_T1.2 | GGGCTCTGACAGGTAGGACC | CIITA | 335 |
| R4505 C2TA_T1.3 | TGTAGGAATCCCAGCCAGGC | CIITA | 336 |
| R4506 C2TA_T1.8 | CCTGGCTCCACGCCCTGCTG | CIITA | 337 |
| R4507 C2TA_T1.9 | GGGAAGCTGAGGGCACGAGG | CIITA | 338 |
| R4508 C2TA_T2.1 | ACAGCGATGCTGACCCCCTG | CIITA | 339 |
| R4509 C2TA_T2.2 | TTAACAGCGATGCTGACCCC | CIITA | 340 |
| R4510 C2TA_T2.3 | TATGACCAGATGGACCTGGC | CIITA | 341 |
| R4511 C2TA_T2.4 | GGGCCCCTAGAAGGTGGCTA | CIITA | 342 |
| R4512 C2TA_T2.5 | TAGGGGCCCCAACTCCATGG | CIITA | 343 |
| R4513 C2TA_T2.6 | AGAAGCTCCAGGTAGCCACC | CIITA | 344 |
| R4514 C2TA_T2.7 | TCCAGCCAGGTCCATCTGGT | CIITA | 345 |
| R4515 C2TA_T2.8 | TTCTCCAGCCAGGTCCATCT | CIITA | 346 |
| R5200 | AGCAGGCTGTTGTGTGACAT | CIITA | 1934 |
| R5201 | CATGTCACACAACAGCCTGC | CIITA | 1935 |
| R5202 | TGTGACATGGAAGGTGATGA | CIITA | 1936 |
| R5203 | ATCACCTTCCATGTCACACA | CIITA | 1937 |
| R5204 | GCATAAGCCTCCCTGGTCTC | CIITA | 1938 |
| R5205 | CAGGACTCCCAGCTGGAGGG | CIITA | 1939 |
| R5206 | CTCAGGCCCTCCAGCTGGGA | CIITA | 1940 |
| R5207 | TGCTGGCATCTCCATACTCT | CIITA | 1941 |
| R5208 | TGCCCAACTTCTGCTGGCAT | CIITA | 1942 |
| R5209 | CTGCCCAACTTCTGCTGGCA | CIITA | 1943 |
| R5210 | TCTGCCCAACTTCTGCTGGC | CIITA | 1944 |
| R5211 | TGACTTTTCTGCCCAACTTC | CIITA | 1945 |
| R5212 | CTGACTTTTCTGCCCAACTT | CIITA | 1946 |
| R5213 | TCTGACTTTTCTGCCCAACT | CIITA | 1947 |
| R5214 | CCAGAGGAGCTTCCGGCAGA | CIITA | 1948 |
| R5215 | AGGTCTGCCGGAAGCTCCTC | CIITA | 1949 |
| R5216 | CGGCAGACCTGAAGCACTGG | CIITA | 1950 |
| R5217 | CAGTGCTTCAGGTCTGCCGG | CIITA | 1951 |
| R5218 | AACAGCGCAGGCAGTGGCAG | CIITA | 1952 |
| R5219 | AACCAGGAGCCAGCCTCCGG | CIITA | 1953 |
| R5220 | TCCAGGCGCATCTGGCCGGA | CIITA | 1954 |
| R5221 | CTCCAGGCGCATCTGGCCGG | CIITA | 1955 |
| R5222 | TCTCCAGGCGCATCTGGCCG | CIITA | 1956 |
| R5223 | CTCCAGTTCCTCGTTGAGCT | CIITA | 1957 |
| R5224 | TCCAGTTCCTCGTTGAGCTG | CIITA | 1958 |
| R5225 | AGGCAGCTCAACGAGGAACT | CIITA | 1959 |
| R5226 | CTCGTTGAGCTGCCTGAATC | CIITA | 1960 |
| R5227 | AGCTGCCTGAATCTCCCTGA | CIITA | 1961 |
| R5228 | GTCCCCACCATCTCCACTCT | CIITA | 1962 |
| R5229 | TCCCCACCATCTCCACTCTG | CIITA | 1963 |
| R5230 | CCAGAGCCCATGGGGCAGAG | CIITA | 1964 |
| R5231 | GCCAGAGCCCATGGGGCAGA | CIITA | 1965 |
| R5232 | CAGCCTCAGAGATTTGCCAG | CIITA | 1966 |
| R5233 | GGAGGCCGTGGACAGTGAAT | CIITA | 1967 |
| R5234 | ACTGTCCACGGCCTCCCAAC | CIITA | 1968 |
| R5235 | GCTCCATCAGCCACTGACCT | CIITA | 1969 |
| R5236 | AGGCATGCTGGGCAGGTCAG | CIITA | 1970 |
| R5237 | CTCGGGAGGTCAGGGCAGGT | CIITA | 1971 |
| R5238 | GCTCGGGAGGTCAGGGCAGG | CIITA | 1972 |
| R5239 | GAGACCTCTCCAGCTGCCGG | CIITA | 1973 |
| R5240 | TTGGAGACCTCTCCAGCTGC | CIITA | 1974 |
| R5241 | GAAGCTTGTTGGAGACCTCT | CIITA | 1975 |
| R5242 | GGAAGCTTGTTGGAGACCTC | CIITA | 1976 |
| R5243 | TGGAAGCTTGTTGGAGACCT | CIITA | 1977 |
| R5244 | TACCGCTCACTGCAGGACAC | CIITA | 1978 |
| R5245 | CTGCTGCTCCTCTCCAGCCT | CIITA | 1979 |
| R5246 | CCGCTCCAGGCTCTTGCTGC | CIITA | 1980 |
| R5247 | TGCCCAGTCCGGGGTGGCCA | CIITA | 1981 |
| R5248 | GGCCAGCTGCCGTTCTGCCC | CIITA | 1982 |
| R5249 | GCAGCCAACAGCACCTCAGC | CIITA | 1983 |
| R5250 | GCTGCCAAGGAGCACCGGCG | CIITA | 1984 |
| R5251 | CCCAGCACAGCAATCACTCG | CIITA | 1985 |
| R5252 | GCCCAGCACAGCAATCACTC | CIITA | 1986 |
| R5253 | CTGTGCTGGGCAAAGCTGGT | CIITA | 1987 |
| R5254 | CCCTGACCAGCTTTGCCCAG | CIITA | 1988 |
| R5255 | GGCTGGGGCAGTGAGCCGGG | CIITA | 1989 |
| R5256 | TGGCCGGCTTCCCCAGTACG | CIITA | 1990 |
| R5257 | CCCAGTACGACTTTGTCTTC | CIITA | 1991 |
| R5258 | GTCTTCTCTGTCCCCTGCCA | CIITA | 1992 |
| R5259 | TCTTCTCTGTCCCCTGCCAT | CIITA | 1993 |
| R5260 | TCTGTCCCCTGCCATTGCTT | CIITA | 1994 |
| R5261 | AAGCAATGGCAGGGGACAGA | CIITA | 1995 |
| R5262 | CTTGAACCGTCCGGGGGATG | CIITA | 1996 |
| R5263 | AACCGTCCGGGGGATGCCTA | CIITA | 1997 |
| R5264 | TCCCTGGGCCCACAGCCACT | CIITA | 1998 |
| R5265 | AAGATGTGGCTGAAAACCTC | CIITA | 1999 |
| R5266 | TCAGCCACATCTTGAAGAGA | CIITA | 2000 |
| R5267 | CAGCCACATCTTGAAGAGAC | CIITA | 2001 |
| R5268 | AGCCACATCTTGAAGAGACC | CIITA | 2002 |
| R5269 | AAGAGACCTGACCGCGTTCT | CIITA | 2003 |
| R5270 | TGCTCATCCTAGACGGCTTC | CIITA | 2004 |
| R5271 | CAGCTCCTCGAAGCCGTCTA | CIITA | 2005 |
| R5272 | CGCTTCCAGCTCCTCGAAGC | CIITA | 2006 |
| R5273 | GAGGAGCTGGAAGCGCAAGA | CIITA | 2007 |
| R5274 | CTGCACAGCACGTGCGGACC | CIITA | 2008 |
| R5275 | TGGAAAAGGCCGGCCAGCAG | CIITA | 2009 |
| R5276 | TTCTGGAAAAGGCCGGCCAG | CIITA | 2010 |
| R5277 | TCCAGAAGAAGCTGCTCCGA | CIITA | 2011 |
| R5278 | CCAGAAGAAGCTGCTCCGAG | CIITA | 2012 |
| R5279 | CAGAAGAAGCTGCTCCGAGG | CIITA | 2013 |
| R5280 | CACCCTCCTCCTCACAGCCC | CIITA | 2014 |
| R5281 | CTCAGGCTCTGGACCAGGCG | CIITA | 2015 |
| R5282 | GAGCTGTCCGGCTTCTCCAT | CIITA | 2016 |
| R5283 | AGCTGTCCGGCTTCTCCATG | CIITA | 2017 |
| R5284 | TCCATGGAGCAGGCCCAGGC | CIITA | 2018 |
| R5285 | GAGAGCTCAGGGATGACAGA | CIITA | 2019 |
| R5286 | AGAGCTCAGGGATGACAGAG | CIITA | 2020 |
| R5287 | GTGCTCTGTCATCCCTGAGC | CIITA | 2021 |
| R5288 | TTCTCAGTCACAGCCACAGC | CIITA | 2022 |
| R5289 | TCAGTCACAGCCACAGCCCT | CIITA | 2023 |
| R5290 | GTGCCGGGCAGTGTGCCAGC | CIITA | 2024 |
| R5291 | TGCCGGGCAGTGTGCCAGCT | CIITA | 2025 |
| R5292 | GCGTCCTCCCCAAGCTCCAG | CIITA | 2026 |
| R5293 | GGGAGGACGCCAAGCTGCCC | CIITA | 2027 |
| R5294 | GCCAGCTCTGCCAGGGCCCC | CIITA | 2028 |
| R5295 | ATGTCTGCGGCCCAGCTCCC | CIITA | 2029 |
| R5392 | GATGTCTGCGGCCCAGCTCC | CIITA | 2030 |
| R5393 | CCATCCGCAGACGTGAGGAC | CIITA | 2031 |
| R5394 | GCCATCGCCCAGGTCCTCAC | CIITA | 2032 |
| R5395 | GGCCATCGCCCAGGTCCTCA | CIITA | 2033 |
| R5396 | GACTAAGCCTTTGGCCATCG | CIITA | 2034 |
| R5397 | GTCCAACACCCACCGCGGGC | CIITA | 2035 |
| R5398 | CAGGAGGAAGCTGGGGAAGG | CIITA | 2036 |
| R5399 | CCCAGCTTCCTCCTGCAATG | CIITA | 2037 |
| R5400 | CTCCTGCAATGCTTCCTGGG | CIITA | 2038 |
| R5401 | CTGGGGGCCCTGTGGCTGGC | CIITA | 2039 |
| R5402 | GCCACTCAGAGCCAGCCACA | CIITA | 2040 |
| R5403 | CGCCACTCAGAGCCAGCCAC | CIITA | 2041 |
| R5404 | ATTTCGCCACTCAGAGCCAG | CIITA | 2042 |
| R5405 | TCCTTGATTTCGCCACTCAG | CIITA | 2043 |
| R5406 | GGGTCAATGCTAGGTACTGC | CIITA | 2044 |
| R5407 | CTTGGGGTCAATGCTAGGTA | CIITA | 2045 |
| R5408 | TTCCTTGGGGTCAATGCTAG | CIITA | 2046 |
| R5409 | ACCCCAAGGAAGAAGAGGCC | CIITA | 2047 |
| R5410 | TCATAGGGCCTCTTCTTCCT | CIITA | 2048 |
| R5411 | CTGGCTGGGCTGATCTTCCA | CIITA | 2049 |
| R5412 | TGGCTGGGCTGATCTTCCAG | CIITA | 2050 |
| R5413 | CAGCCTCCCGCCCGCTGCCT | CIITA | 2051 |
| R5414 | CTGTCCACCGAGGCAGCCGC | CIITA | 2052 |
| R5415 | TGCTTCCTGTCCACCGAGGC | CIITA | 2053 |
| R5416 | AGGTACCTCGCAAGCACCTT | CIITA | 2054 |
| R5417 | CGAGGTACCTGAAGCGGCTG | CIITA | 2055 |
| R5418 | CAGCCTCCTCGGCCTCGTGG | CIITA | 2056 |
| R5419 | GGCAGCACGTGGTACAGGAG | CIITA | 2057 |
| R5420 | GCAGCACGTGGTACAGGAGC | CIITA | 2058 |
| R5421 | TCTGGGCACCCGCCTCACGC | CIITA | 2059 |
| R5422 | CTGGGCACCCGCCTCACGCC | CIITA | 2060 |
| R5423 | TGGGCACCCGCCTCACGCCT | CIITA | 2061 |
| R5424 | CCCAGTACATGTGCATCAGG | CIITA | 2062 |
| R5425 | GCCCGCCGCCTCCAAGGCCT | CIITA | 2063 |
| R5426 | GAGGCGGCGGGCCAAGACTT | CIITA | 2064 |
| R5427 | TCCCTGGACCTCCGCAGCAC | CIITA | 2065 |
| R5428 | GCCCCTCTGGATTGGGGAGC | CIITA | 2066 |
| R5429 | CCCCTCTGGATTGGGGAGCC | CIITA | 2067 |
| R5430 | GGGAGCCTCGTGGGACTCAG | CIITA | 2068 |
| R5431 | GTCTCCCCATGCTGCTGCAG | CIITA | 2069 |
| R5432 | TCCTCTGCTGCCTGAAGTAG | CIITA | 2070 |
| R5433 | AGGCAGCAGAGGAGAAGTTC | CIITA | 2071 |
| R5434 | AAAGGCTCGATGGTGAACTT | CIITA | 2072 |
| R5435 | GAAAGGCTCGATGGTGAACT | CIITA | 2073 |
| R5436 | ACCATCGAGCCTTTCAAAGC | CIITA | 2074 |
| R5437 | GCTTTGAAAGGCTCGATGGT | CIITA | 2075 |
| R5438 | AGGGACTTGGCTTTGAAAGG | CIITA | 2076 |
| R5439 | CAAAGCCAAGTCCCTGAAGG | CIITA | 2077 |
| R5440 | AAAGCCAAGTCCCTGAAGGA | CIITA | 2078 |
| R5441 | CACATCCTTCAGGGACTTGG | CIITA | 2079 |
| R5442 | CCAGGTCTTCCACATCCTTC | CIITA | 2080 |
| R5443 | CCCAGGTCTTCCACATCCTT | CIITA | 2081 |
| R5444 | CTCGGAAGACACAGCTGGGG | CIITA | 2082 |
| R5445 | GGTCCCGAACAGCAGGGAGC | CIITA | 2083 |
| R5446 | AGGTCCCGAACAGCAGGGAG | CIITA | 2084 |
| R5447 | TTTAGGTCCCGAACAGCAGG | CIITA | 2085 |
| R5448 | CTTTAGGTCCCGAACAGCAG | CIITA | 2086 |
| R5449 | GGGACCTAAAGAAACTGGAG | CIITA | 2087 |
| R5450 | GGGAAAGCCTGGGGGCCTGA | CIITA | 2088 |
| R5451 | GGGGAAAGCCTGGGGGCCTG | CIITA | 2089 |
| R5452 | CCCCAAACTGGTGCGGATCC | CIITA | 2090 |
| R5453 | CCCAAACTGGTGCGGATCCT | CIITA | 2091 |
| R5454 | TTCTCACTCAGCGCATCCAG | CIITA | 2092 |
| R5455 | AGCTGGGGGAAGGTGGCTGA | CIITA | 2093 |
| R5456 | CCCCAGCTGAAGTCCTTGGA | CIITA | 2094 |
| R5457 | CAAGGACTTCAGCTGGGGGA | CIITA | 2095 |
| R5458 | CCAAGGACTTCAGCTGGGGG | CIITA | 2096 |
| R5459 | AGGGTTTCCAAGGACTTCAG | CIITA | 2097 |
| R5460 | TAGGCACCCAGGTCAGTGAT | CIITA | 2098 |
| R5461 | GTAGGCACCCAGGTCAGTGA | CIITA | 2099 |
| R5462 | GCTCGCTGCATCCCTGCTCA | CIITA | 2100 |
| R5463 | GCCTGAGCAGGGATGCAGCG | CIITA | 2101 |
| R5464 | TACAATAACTGCATCTGCGA | CIITA | 2102 |
| R5465 | GCTCGTGTGCTTCCGGACAT | CIITA | 2103 |
| R5466 | CGGACATGGTGTCCCTCCGG | CIITA | 2104 |
| R5467 | ACGGCTGCCGGGGCCCAGCA | CIITA | 2105 |
| R5468 | GGAGGTGTCCTCATGTGGAG | CIITA | 2106 |
| R5469 | CTGGACACTGAATGGGATGG | CIITA | 2107 |
| R5470 | AGTGTCCAGGAACACCTGCA | CIITA | 2108 |
| R5471 | CAGGTGTTCCTGGACACTGA | CIITA | 2109 |
| R5472 | TTGCAGGTGTTCCTGGACAC | CIITA | 2110 |
| R5473 | ACGGATCAGCCTGAGATGAT | CIITA | 2111 |

TABLE E
spacer sequences of gRNAs targeting mouse PCSK9

| | Spacer sequence | | |
|---|---|---|---|
| Name | (5′ → 3′) | Target | SEQ ID NO |
| R4238 | CCGCUGUUGCCGCCGCUGCU | PCSK9 | 347 |
| R4239 | CCGCCGCUGCUGCUGCUGUU | PCSK9 | 348 |
| R4240 | CUGCUACUGUGCCCCACCGG | PCSK9 | 349 |
| R4241 | AUAAUCUCCAUCCUCGUCCU | PCSK9 | 350 |
| R4242 | UGAAGAGCUGAUGCUCGCCC | PCSK9 | 351 |
| R4243 | GAGCAACGGCGGAAGGUGGC | PCSK9 | 352 |
| R4244 | CUGGCAGCCUCCAGGCCUCC | PCSK9 | 353 |
| R4245 | UGGUGCUGAUGGAGGAGACC | PCSK9 | 354 |
| R4246 | AAUCUGUAGCCUCUGGGUCU | PCSK9 | 355 |
| R4247 | UUCAAUCUGUAGCCUCUGGG | PCSK9 | 356 |
| R4248 | GUUCAAUCUGUAGCCUCUGG | PCSK9 | 357 |
| R4249 | AACAAACUGCCCACCGCCUG | PCSK9 | 358 |
| R4250 | AUGACAUAGCCCCGGCGGGC | PCSK9 | 359 |
| R4251 | UACAUAUCUUUUAUGACCUC | PCSK9 | 360 |
| R4252 | UAUGACCUCUUCCCUGGCUU | PCSK9 | 361 |
| R4253 | AUGACCUCUUCCCUGGCUUC | PCSK9 | 362 |
| R4254 | UGACCUCUUCCCUGGCUUCU | PCSK9 | 363 |
| R4255 | ACCAAGAAGCCAGGGAAGAG | PCSK9 | 364 |
| R4256 | CCUGGCUUCUUGGUGAAGAU | PCSK9 | 365 |
| R4257 | UUGGUGAAGAUGAGCAGUGA | PCSK9 | 366 |
| R4258 | GUGAAGAUGAGCAGUGACCU | PCSK9 | 367 |
| R4259 | CCCCAUGUGGAGUACAUUGA | PCSK9 | 368 |
| R4260 | CUCAAUGUACUCCACAUGGG | PCSK9 | 369 |
| R4261 | AGGAAGACUCCUUUGUCUUC | PCSK9 | 370 |
| R4262 | GUCUUCGCCCAGAGCAUCCC | PCSK9 | 371 |
| R4263 | UCUUCGCCCAGAGCAUCCCA | PCSK9 | 372 |
| R4264 | GCCCAGAGCAUCCCAUGGAA | PCSK9 | 373 |
| R4265 | CAUGGGAUGCUCUGGGCGAA | PCSK9 | 374 |
| R4266 | GCUCCAGGUUCCAUGGGAUG | PCSK9 | 375 |
| R4267 | UCCCAGCAUGGCACCAGACA | PCSK9 | 376 |
| R4268 | CUCUGUCUGGUGCCAUGCUG | PCSK9 | 377 |
| R4269 | GAUACCAGCAUCCAGGGUGC | PCSK9 | 378 |
| R4270 | AGGGCAGGGUCACCAUCACC | PCSK9 | 379 |
| R4271 | AAGUCGGUGAUGGUGACCCU | PCSK9 | 380 |
| R4272 | AACAGCGUGCCGGAGGAGGA | PCSK9 | 381 |
| R4273 | GCCACACCAGCAUCCCGGCC | PCSK9 | 382 |
| R4274 | AGCACACGCAGGCUGUGCAG | PCSK9 | 383 |
| R4275 | ACAGUUGAGCACACGCAGGC | PCSK9 | 384 |
| R4276 | CCUUGACAGUUGAGCACACG | PCSK9 | 385 |
| R4277 | GCUGACUCUUCCGAAUAAAC | PCSK9 | 386 |
| R4278 | AUUCGGAAGAGUCAGCUAAU | PCSK9 | 387 |
| R4279 | UUCGGAAGAGUCAGCUAAUC | PCSK9 | 388 |
| R4280 | GGAAGAGUCAGCUAAUCCAG | PCSK9 | 389 |
| R4281 | UGCUGCCCCUGGCCGGUGGG | PCSK9 | 390 |
| R4282 | AGGAUGCGGCUAUACCCACC | PCSK9 | 391 |
| R4283 | CCAGCUGCUGCAACCAGCAC | PCSK9 | 392 |
| R4284 | CAGCAGCUGGGAACUUCCGG | PCSK9 | 393 |
| R4285 | CGGGACGACGCCUGCCUCUA | PCSK9 | 394 |
| R4286 | GUGGCCCCGACUGUGAUGAC | PCSK9 | 395 |
| R4287 | CCUUGGGGACUUUGGGGACU | PCSK9 | 396 |
| R4288 | GUCCCCAAAGUCCCCAAGGU | PCSK9 | 397 |
| R4289 | GGGACUUUGGGGACUAAUUU | PCSK9 | 398 |
| R4290 | GGGGACUAAUUUUGGACGCU | PCSK9 | 399 |
| R4291 | GGGACUAAUUUUGGACGCUG | PCSK9 | 400 |
| R4292 | UGGACGCUGUGUGGAUCUCU | PCSK9 | 401 |
| R4293 | GGACGCUGUGUGGAUCUCUU | PCSK9 | 402 |
| R4294 | GACGCUGUGUGGAUCUCUUU | PCSK9 | 403 |
| R4295 | CCGGGGGCAAAGAGAUCCAC | PCSK9 | 404 |
| R4296 | GCCCCCGGGAAGGACAUCAU | PCSK9 | 405 |
| R4297 | CCCCCGGGAAGGACAUCAUC | PCSK9 | 406 |
| R4298 | AUGUCACAGAGUGGGACCUC | PCSK9 | 407 |
| R4299 | UGGCUCGGAUGCUGAGCCGG | PCSK9 | 408 |
| R4300 | CCCUGGCCGAGCUGCGGCAG | PCSK9 | 409 |
| R4301 | GUAGAGAAGUGGAUCAGCCU | PCSK9 | 410 |
| R4302 | GGUAGAGAAGUGGAUCAGCC | PCSK9 | 411 |
| R4303 | UCUACCAAAGACGUCAUCAA | PCSK9 | 412 |
| R4304 | AUGACGUCUUUGGUAGAGAA | PCSK9 | 413 |
| R4305 | CCUGAGGACCAGCAGGUGCU | PCSK9 | 414 |
| R4306 | GGGGUCAGCACCUGCUGGUC | PCSK9 | 415 |
| R4307 | GAGUGGGCCCCGAGUGUGCC | PCSK9 | 416 |
| R4308 | UGGGGCACAGCGGGCUGUAG | PCSK9 | 417 |
| R4309 | UCCAGGAGCGGGAGGCGUCG | PCSK9 | 418 |
| R4310 | CAGACCUGCUGGCCUCCUAU | PCSK9 | 419 |
| R4311 | AGGGCCUUGCAGACCUGCUG | PCSK9 | 420 |
| R4312 | GGGGGUGAGGGUGUCUAUGC | PCSK9 | 421 |
| R4313 | GGGGUGAGGGUGUCUAUGCC | PCSK9 | 422 |
| R4314 | GCACGGGGAACCAGGCAGCA | PCSK9 | 423 |
| R4315 | CCCGUGCCAACUGCAGCAUC | PCSK9 | 424 |
| R4316 | UGGAUGCUGCAGUUGGCACG | PCSK9 | 425 |
| R4317 | UGGUGGCAGUGGACAUGGGU | PCSK9 | 426 |
| R4318 | CACUUCCCAAUGGAAGCUGC | PCSK9 | 427 |
| R4319 | CAUUGGGAAGUGGAAGACCU | PCSK9 | 428 |
| R4320 | GGAAGUGGAAGACCUUAGUG | PCSK9 | 429 |
| R4321 | GUGUCCGGAGGCAGCCUGCG | PCSK9 | 430 |
| R4322 | GCCACCAGGCGGCCAGUGUC | PCSK9 | 431 |
| R4323 | CUGCUGCCAUGCCCCAGGGC | PCSK9 | 432 |
| R4324 | CAGCCCUGGGGCAUGGCAGC | PCSK9 | 433 |
| R4325 | CAUUCCAGCCCUGGGGCAUG | PCSK9 | 434 |
| R4326 | GCAUUCCAGCCCUGGGGCAU | PCSK9 | 435 |
| R4327 | UGCAUUCCAGCCCUGGGGCA | PCSK9 | 436 |
| R4328 | AUUUUGCAUUCCAGCCCUGG | PCSK9 | 437 |
| R4329 | CAUCCAGUCAGGGUCCAUCC | PCSK9 | 438 |
| R4330 | UCCACGCUGUAGGCUCCCAG | PCSK9 | 439 |
| R4331 | CCACACACAGGUUGUCCACG | PCSK9 | 440 |
| R4332 | UCCACUGGUCCUGUCUGCUC | PCSK9 | 441 |
| R4333 | CUGAAGGCCGGCUCCGGCAG | PCSK9 | 442 |

TABLE F
spacer sequences of gRNAs targets Bak1 in CHO
cells

| | Spacer sequence | SEQ |
|---|---|---|
| | (5′ → 3′), | ID |
| Name | shown as DNA | NO |
| R2452_Bak1_CasPhi_1 | GAAGCTATGTTTTCCATCTC | 443 |
| R2453_Bak1_CasPhi_2 | GCAGGGGCAGCCGCCCCCTG | 444 |
| R2454_Bak1_CasPhi_3 | CTCCTAGAACCCAACAGGTA | 445 |
| R2455_Bak1_CasPhi_4 | GAAAGACCTCCTCTGTGTCC | 446 |
| R2456_Bak1_CasPhi_5 | TCCATCTCGGGGTTGGCAGG | 447 |
| R2457_Bak1_CasPhi_6 | TTCCTGATGGTGGAGATGGA | 448 |
| R2849 Bakl nsd_sg1 | CTGACTCCCAGCTCTGACCC | 449 |
| R2850_Bak1_nsd_sg2 | TGGGGTCAGAGCTGGGAGTC | 450 |
| R2851_Bak1_nsd_sg3 | GAAAGACCTCCTCTGTGTCC | 451 |
| R2852_Bak1_nsd_sg4 | CGAAGCTATGTTTTCCATCT | 452 |
| R2853_Bak1_nsd_sg5 | GAAGCTATGTTTTCCATCTC | 453 |
| R2854_Bak1_nsd_sg6 | TCCATCTCCACCATCAGGAA | 454 |
| R2855_Bak1_nsd_sg7 | CCATCTCCACCATCAGGAAC | 455 |
| R2856_Bak1_nsd_sg8 | CTGATGGTGGAGATGGAAAA | 456 |
| R2857 Bakl nsd_sg9 | CATCTCCACCATCAGGAACA | 457 |
| R2858_Bak1_nsd_sg10 | TTCCTGATGGTGGAGATGGA | 458 |
| R2859_Bak1_nsd_sg11 | GCAGGGGCAGCCGCCCCCTG | 459 |
| R2860_Bak1_nsd_sg12 | TCCATCTCGGGGTTGGCAGG | 460 |
| R2861_Bak1_nsd_sg13 | TAGGAGCAAATTGTCCATCT | 461 |
| R2862_Bak1_nsd sg14 | GGTTCTAGGAGCAAATTGTC | 462 |
| R2863_Bak1_nsd_sg15 | GCTCCTAGAACCCAACAGGT | 463 |
| R2864_Bak1_nsd_sg16 | CTCCTAGAACCCAACAGGTA | 464 |
| R3977_Bak1_exon1_sg1 | TCCAGACGCCATCTTTCAGG | 465 |
| R3978_Bak1_exon1_sg2 | TGGTAAGAGTCCTCCTGCCC | 466 |
| R3979_Bak1_exon3_sg1 | TTACAGCATCTTGGGTCAGG | 467 |
| R3980_Bak1_exon3_sg2 | GGTCAGGTGGGCCGGCAGCT | 468 |
| R3981_Bak1_exon3_sg3 | CTATCATTGGAGATGACATT | 469 |
| R3982_Bak1_exon3_sg4 | GAGATGACATTAACCGGAGA | 470 |
| R3983_Bak1_exon3_sg5 | TGGAACTCTGTGTCGTATCT | 471 |
| R3984_Bak1_exon3_sg6 | CAGAATTTACTGGAGCAGCT | 472 |
| R3985_Bak1_exon3_sg7 | ACTGGAGCAGCTGCAGCCCA | 473 |
| R3986_Bak1_exon3_sg8 | CCAGCTGTGGGCTGCAGCTG | 474 |
| R3987_Bak1_exon3_sg9 | GTAGGCATTCCCAGCTGTGG | 475 |
| R3988_Bak1_exon3_sg10 | GTGAAGAGTTCGTAGGCATT | 476 |
| R3989_Bak1_exon3_sg11 | ACCAAGATTGCCTCCAGGTA | 477 |
| R3990_Bak1_exon3_sg12 | CCTCCAGGTACCCACCACCA | 478 |

TABLE G
spacer sequences of gRNAs targeting Bax in CHO
cells

| | Spacer sequence | SEQ |
|---|---|---|
| | (5′ → 3′), | ID |
| Name | shown as DNA | NO |
| R2458_Bax_CasPhi_1 | CTAATGTGGATACTAACTCC | 479 |
| R2459_Bax_CasPhi_2 | TTCCGTGTGGCAGCTGACAT | 480 |
| R2460_Bax_CasPhi_3 | CTGATGGCAACTTCAACTGG | 481 |
| R2461_Bax_CasPhi_4 | TACTTTGCTAGCAAACTGGT | 482 |
| R2462_Bax_CasPhi_5 | AGCACCAGTTTGCTAGCAAA | 483 |
| R2463_Bax_CasPhi_6 | AACTGGGGCCGGGTTGTTGC | 484 |
| R2865_Bax_nsd sg1 | TTCTCTTTCCTGTAGGATGA | 485 |
| R2866_Bax_nsd_sg2 | TCTTTCCTGTAGGATGATTG | 486 |
| R2867_Bax_nsd_sg3 | CCTGTAGGATGATTGCTAAT | 487 |
| R2868_Bax_nsd_sg4 | CTGTAGGATGATTGCTAATG | 488 |
| R2869_Bax_nsd_sg5 | CTAATGTGGATACTAACTCC | 489 |
| R2870_Bax_nsd_sg6 | TTCCGTGTGGCAGCTGACAT | 490 |
| R2871_Bax_nsd_sg7 | CGTGTGGCAGCTGACATGTT | 491 |
| R2872_Bax_nsd_sg8 | CCATCAGCAAACATGTCAGC | 492 |
| R2873_Bax_nsd_sg9 | AAGTTGCCATCAGCAAACAT | 493 |
| R2874_Bax_nsd_sg10 | GCTGATGGCAACTTCAACTG | 494 |
| R2875_Bax_nsd_sg11 | CTGATGGCAACTTCAACTGG | 495 |
| R2876_Bax_nsd_sg12 | AACTGGGGCCGGGTTGTTGC | 496 |
| R2877_Bax_nsd_sg13 | TTGCCCTTTTCTACTTTGCT | 497 |
| R2878_Bax_nsd sg14 | CCCTTTTCTACTTTGCTAGC | 498 |
| R2879_Bax_nsd sg15 | CTAGCAAAGTAGAAAAGGGC | 499 |
| R2880_Bax_nsd sg16 | GCTAGCAAAGTAGAAAAGGG | 500 |
| R2881_Bax_nsd_sg17 | TCTACTTTGCTAGCAAACTG | 501 |
| R2882_Bax_nsd_sg18 | CTACTTTGCTAGCAAACTGG | 502 |
| R2883_Bax_nsd_sg19 | TACTTTGCTAGCAAACTGGT | 503 |
| R2884_Bax_nsd_sg20 | GCTAGCAAACTGGTGCTCAA | 504 |
| R2885_Bax_nsd_sg21 | CTAGCAAACTGGTGCTCAAG | 505 |
| R2886_Bax_nsd_sg22 | AGCACCAGTTTGCTAGCAAA | 506 |

TABLE H
spacer sequences of gRNAs targeting Fut8 in CHO
cells

| | Spacer sequence | SEQ |
|---|---|---|
| | (5′ → 3′), | ID |
| Name | shown as DNA | NO |
| R2464_Fut8_CasPhi_1 | CCACTTTGTCAGTGCGTCTG | 507 |
| R2465_Fut8 casPhi 2 | CTCAATGGGATGGAAGGCTG | 508 |
| R2466_Fut8_CasPhi_3 | AGGAATACATGGTACACGTT | 509 |
| R2467_Fut8_CasPhi_4 | AAGAACATTTTCAGCTTCTC | 510 |
| R2468_Fut8_CasPhi_5 | ATCCACTTTCATTCTGCGTT | 511 |
| R2469_Fut8_CasPhi_6 | TTTGTTAAAGGAGGCAAAGA | 512 |
| R2887_Fut8 nsd sg1 | TCCCCAGAGTCCATGTCAGA | 513 |
| R2888_Fut8 nsd_sg2 | TCAGTGCGTCTGACATGGAC | 514 |
| R2889_Fut8_nsd_sg3 | GTCAGTGCGTCTGACATGGA | 515 |
| R2890_Fut8 nsd_sg4 | CCACTTTGTCAGTGCGTCTG | 516 |
| R2891_Fut8_nsd_sg5 | TGTTCCCACTTTGTCAGTGC | 517 |
| R2892_Fut8_nsd_sg6 | CTCAATGGGATGGAAGGCTG | 518 |
| R2893_Fut8 nsd_sg7 | CATCCCATTGAGGAATACAT | 519 |
| R2894_Fut8_nsd_sg8 | AGGAATACATGGTACACGTT | 520 |
| R2895_Fut8_nsd_sg9 | AACGTGTACCATGTATTCCT | 521 |
| R2896_Fut8 nsd sg10 | TTCAACGTGTACCATGTATT | 522 |
| R2897_Fut8 nsd_sg11 | AAGAACATTTTCAGCTTCTC | 523 |
| R2898_Fut8_nsd_sg12 | GAGAAGCTGAAAATGTTCTT | 524 |
| R2899_Fut8 nsd_sg13 | TCAGCTTCTCGAACGCAGAA | 525 |
| R2900_Fut8_nsd_sg14 | CAGCTTCTCGAACGCAGAAT | 526 |
| R2901_Fut8_nsd_sg15 | TGCGTTCGAGAAGCTGAAAA | 527 |
| R2902_Fut8_nsd_sg16 | AGCTTCTCGAACGCAGAATG | 528 |
| R2903_Fut8 nsd sg17 | ATTCTGCGTTCGAGAAGCTG | 529 |
| R2904_Fut8_nsd_sg18 | CATTCTGCGTTCGAGAAGCT | 530 |
| R2905_Fut8_nsd_sg19 | TCGAACGCAGAATGAAAGTG | 531 |
| R2906_Fut8_nsd_sg20 | ATCCACTTTCATTCTGCGTT | 532 |
| R2907_Fut8 nsd_sg21 | TATCCACTTTCATTCTGCGT | 533 |
| R2908_Fut8 nsd sg22 | TTATCCACTTTCATTCTGCG | 534 |
| R2909_Fut8 nsd_sg23 | TTTATCCACTTTCATTCTGC | 535 |
| R2910_Fut8_nsd_sg24 | TTTTATCCACTTTCATTCTG | 536 |
| R2911_Fut8_nsd_sg25 | AACAAAGAAGGGTCATCAGT | 537 |
| R2912_Fut8 nsd_sg26 | CCTCCTTTAACAAAGAAGGG | 538 |
| R2913_Fut8_nsd_sg27 | GCCTCCTTTAACAAAGAAGG | 539 |
| R2914_Fut8 nsd_sg28 | TTTGTTAAAGGAGGCAAAGA | 540 |
| R2915_Fut8_nsd_sg29 | GTTAAAGGAGGCAAAGACAA | 541 |
| R2916_Fut8 nsd_sg30 | TTAAAGGAGGCAAAGACAAA | 542 |
| R2917_Fut8 nsd_sg31 | TCTTTGCCTCCTTTAACAAA | 543 |
| R2918_Fut8_nsd_sg32 | GTCTTTGCCTCCTTTAACAA | 544 |
| R2919_Fut8 nsd_sg33 | GTCTAACTTACTTTGTCTTT | 545 |
| R2920_Fut8_nsd_sg34 | TTGGTCTAACTTACTTTGTC | 546 |

TABLE I
CasΦ.12 gRNAs targeting human TRAC in T cells

| Name | Repeat + spacer RNA Sequence (5′ → 3′), shown as DNA | SEQ ID NO |
|---|---|---|
| R3040_ | CTTTCAAGACTAATAGATTGCTCCTTACGAGGAGAC | 547 |
| CasPhi12 | TGGATATCTGTGGGACAAGA | |
| R3041_ | CTTTCAAGACTAATAGATTGCTCCTTACGAGGAGAC | 548 |
| CasPhi12 | TCCCACAGATATCCAGAACC | |
| R3042_ | CTTTCAAGACTAATAGATTGCTCCTTACGAGGAGAC | 549 |
| CasPhi12 | GAGTCTCTCAGCTGGTACAC | |
| R3043_ | CTTTCAAGACTAATAGATTGCTCCTTACGAGGAGAC | 550 |
| CasPhi12 | AGAGTCTCTCAGCTGGTACA | |
| R3044_ | CTTTCAAGACTAATAGATTGCTCCTTACGAGGAGAC | 551 |
| CasPhi12 | TCACTGGATTTAGAGTCTCT | |
| R3045_ | CTTTCAAGACTAATAGATTGCTCCTTACGAGGAGAC | 552 |
| CasPhi12 | AGAATCAAAATCGGTGAATA | |
| R3046_ | CTTTCAAGACTAATAGATTGCTCCTTACGAGGAGAC | 553 |
| CasPhi12 | GAGAATCAAAATCGGTGAAT | |
| R3047_ | CTTTCAAGACTAATAGATTGCTCCTTACGAGGAGAC | 554 |
| CasPhi12 | ACCGATTTTGATTCTCAAAC | |
| R3048_ | CTTTCAAGACTAATAGATTGCTCCTTACGAGGAGAC | 555 |
| CasPhi12 | TTTGAGAATCAAAATCGGTG | |
| R3049_ | CTTTCAAGACTAATAGATTGCTCCTTACGAGGAGAC | 556 |
| CasPhi12 | GTTTGAGAATCAAAATCGGT | |
| R3050_ | CTTTCAAGACTAATAGATTGCTCCTTACGAGGAGAC | 557 |
| CasPhi12 | TGATTCTCAAACAAATGTGT | |
| R3051_ | CTTTCAAGACTAATAGATTGCTCCTTACGAGGAGAC | 558 |
| CasPhi12 | GATTCTCAAACAAATGTGTC | |
| R3052_ | CTTTCAAGACTAATAGATTGCTCCTTACGAGGAGAC | 559 |
| CasPhi12 | ATTCTCAAACAAATGTGTCA | |
| R3053__ | CTTTCAAGACTAATAGATTGCTCCTTACGAGGAGAC | 560 |
| CasPhi12 | TGACACATTTGTTTGAGAAT | |
| R3054_ | CTTTCAAGACTAATAGATTGCTCCTTACGAGGAGAC | 561 |
| CasPhi12 | TCAAACAAATGTGTCACAAA | |
| R3055_ | CTTTCAAGACTAATAGATTGCTCCTTACGAGGAGAC | 562 |
| CasPhi12 | GTGACACATTTGTTTGAGAA | |
| R3056_ | CTTTCAAGACTAATAGATTGCTCCTTACGAGGAGAC | 563 |
| CasPhi12 | CTTTGTGACACATTTGTTTG | |
| R3057_ | CTTTCAAGACTAATAGATTGCTCCTTACGAGGAGAC | 564 |
| CasPhi12 | TGATGTGTATATCACAGACA | |
| R3058_ | CTTTCAAGACTAATAGATTGCTCCTTACGAGGAGAC | 565 |
| CasPhi12 | TCTGTGATATACACATCAGA | |
| R3059_ | CTTTCAAGACTAATAGATTGCTCCTTACGAGGAGAC | 566 |
| CasPhi12 | GTCTGTGATATACACATCAG | |
| R3060_ | CTTTCAAGACTAATAGATTGCTCCTTACGAGGAGAC | 567 |
| CasPhi12 | TGTCTGTGATATACACATCA | |
| R3061_ | CTTTCAAGACTAATAGATTGCTCCTTACGAGGAGAC | 568 |
| CasPhi12 | AAGTCCATAGACCTCATGTC | |
| R3062_ | CTTTCAAGACTAATAGATTGCTCCTTACGAGGAGAC | 569 |
| CasPhi12 | CTCTTGAAGTCCATAGACCT | |
| R3063_ | CTTTCAAGACTAATAGATTGCTCCTTACGAGGAGAC | 570 |
| CasPhi12 | AAGAGCAACAGTGCTGTGGC | |
| R3064_ | CTTTCAAGACTAATAGATTGCTCCTTACGAGGAGAC | 571 |
| CasPhi12 | CTCCAGGCCACAGCACTGTT | |
| R3065_ | CTTTCAAGACTAATAGATTGCTCCTTACGAGGAGAC | 572 |
| CasPhi12 | TTGCTCCAGGCCACAGCACT | |
| R3066_ | CTTTCAAGACTAATAGATTGCTCCTTACGAGGAGAC | 573 |
| CasPhi12 | GTTGCTCCAGGCCACAGCAC | |
| R3067_ | CTTTCAAGACTAATAGATTGCTCCTTACGAGGAGAC | 574 |
| CasPhi12 | CACATGCAAAGTCAGATTTG | |
| R3068_ | CTTTCAAGACTAATAGATTGCTCCTTACGAGGAGAC | 575 |
| CasPhi12 | GCACATGCAAAGTCAGATTT | |
| R3069_ | CTTTCAAGACTAATAGATTGCTCCTTACGAGGAGAC | 576 |
| CasPhi12 | GCATGTGCAAACGCCTTCAA | |
| R3070_ | CTTTCAAGACTAATAGATTGCTCCTTACGAGGAGAC | 577 |
| CasPhi12 | AAGGCGTTTGCACATGCAAA | |
| R3071_ | CTTTCAAGACTAATAGATTGCTCCTTACGAGGAGAC | 578 |
| CasPhi12 | CATGTGCAAACGCCTTCAAC | |
| R3072_ | CTTTCAAGACTAATAGATTGCTCCTTACGAGGAGAC | 579 |
| CasPhi12 | TTGAAGGCGTTTGCACATGC | |
| R3073_ | CTTTCAAGACTAATAGATTGCTCCTTACGAGGAGAC | 580 |
| CasPhi12 | AACAACAGCATTATTCCAGA | |
| R3074_ | CTTTCAAGACTAATAGATTGCTCCTTACGAGGAGAC | 581 |
| CasPhi12 | TGGAATAATGCTGTTGTTGA | |
| R3075_ | CTTTCAAGACTAATAGATTGCTCCTTACGAGGAGAC | 582 |
| CasPhi12 | TTCCAGAAGACACCTTCTTC | |
| R3076_ | CTTTCAAGACTAATAGATTGCTCCTTACGAGGAGAC | 583 |
| CasPhi12 | CAGAAGACACCTTCTTCCCC | |
| R3077_ | CTTTCAAGACTAATAGATTGCTCCTTACGAGGAGAC | 584 |
| CasPhi12 | CCTGGGCTGGGGAAGAAGGT | |
| R3078_ | CTTTCAAGACTAATAGATTGCTCCTTACGAGGAGAC | 585 |
| CasPhi12 | TTCCCCAGCCCAGGTAAGGG | |
| R3079_ | CTTTCAAGACTAATAGATTGCTCCTTACGAGGAGAC | 586 |
| CasPhi12 | CCCAGCCCAGGTAAGGGCAG | |
| R3080_ | CTTTCAAGACTAATAGATTGCTCCTTACGAGGAGAC | 587 |
| CasPhi12 | TAAAAGGAAAAACAGACATT | |
| R3081_ | CTTTCAAGACTAATAGATTGCTCCTTACGAGGAGAC | 588 |
| CasPhi12 | CTAAAAGGAAAAACAGACAT | |
| R3082_ | CTTTCAAGACTAATAGATTGCTCCTTACGAGGAGAC | 589 |
| CasPhi12 | TTCCTTTTAGAAAGTTCCTG | |
| R3083_ | CTTTCAAGACTAATAGATTGCTCCTTACGAGGAGAC | 590 |
| CasPhi12 | TCCTTTTAGAAAGTTCCTGT | |
| R3084_ | CTTTCAAGACTAATAGATTGCTCCTTACGAGGAGAC | 591 |
| CasPhi12 | CCTTTTAGAAAGTTCCTGTG | |
| R3085_ | CTTTCAAGACTAATAGATTGCTCCTTACGAGGAGAC | 592 |
| CasPhi12 | CTTTTAGAAAGTTCCTGTGA | |
| R3086_ | CTTTCAAGACTAATAGATTGCTCCTTACGAGGAGAC | 593 |
| CasPhi12 | TAGAAAGTTCCTGTGATGTC | |
| R3136_ | CTTTCAAGACTAATAGATTGCTCCTTACGAGGAGAC | 594 |
| CasPhi12 | AGAAAGTTCCTGTGATGTCA | |
| R3137_ | CTTTCAAGACTAATAGATTGCTCCTTACGAGGAGAC | 595 |
| CasPhi12 | GAAAGTTCCTGTGATGTCAA | |
| R3138_ | CTTTCAAGACTAATAGATTGCTCCTTACGAGGAGAC | 596 |
| CasPhi12 | ACATCACAGGAACTTTCTAA | |
| R3139_ | CTTTCAAGACTAATAGATTGCTCCTTACGAGGAGAC | 597 |
| CasPhi12 | CTGTGATGTCAAGCTGGTCG | |
| R3140_ | CTTTCAAGACTAATAGATTGCTCCTTACGAGGAGAC | 598 |
| CasPhi12 | TCGACCAGCTTGACATCACA | |
| R3141_ | CTTTCAAGACTAATAGATTGCTCCTTACGAGGAGAC | 599 |
| CasPhi12 | CTCGACCAGCTTGACATCAC | |
| R3142_ | CTTTCAAGACTAATAGATTGCTCCTTACGAGGAGAC | 600 |
| CasPhi12 | TCTCGACCAGCTTGACATCA | |
| R3143_ | CTTTCAAGACTAATAGATTGCTCCTTACGAGGAGAC | 601 |
| CasPhi12 | AAAGCTTTTCTCGACCAGCT | |
| R3144_ | CTTTCAAGACTAATAGATTGCTCCTTACGAGGAGAC | 602 |
| CasPhi12 | CAAAGCTTTTCTCGACCAGC | |
| R3145_ | CTTTCAAGACTAATAGATTGCTCCTTACGAGGAGAC | 603 |
| CasPhi12 | CCTGTTTCAAAGCTTTTCTC | |
| R3146_ | CTTTCAAGACTAATAGATTGCTCCTTACGAGGAGAC | 604 |
| CasPhi12 | GAAACAGGTAAGACAGGGGT | |
| R3147_ | CTTTCAAGACTAATAGATTGCTCCTTACGAGGAGAC | 605 |
| CasPhi12 | AAACAGGTAAGACAGGGGTC | |

TABLE J
CasΦ.32 gRNAs targeting human TRAC in T cells

| Name | Repeat + spacer RNA Sequence (5′ → 3′), shown as DNA | SEQ ID NO |
|---|---|---|
| R3040_ | GCTGGGGACCGATCCTGATTGCTCGCTGCGGCGAGA | 606 |
| CasPhi32 | CTGGATATCTGTGGGACAAGA | |
| R3041_ | GCTGGGGACCGATCCTGATTGCTCGCTGCGGCGAGA | 607 |
| CasPhi32 | CTCCCACAGATATCCAGAACC | |
| R3042_ | GCTGGGGACCGATCCTGATTGCTCGCTGCGGCGAGA | 608 |
| CasPhi32 | CGAGTCTCTCAGCTGGTACAC | |
| R3043_ | GCTGGGGACCGATCCTGATTGCTCGCTGCGGCGAGA | 609 |
| CasPhi32 | CAGAGTCTCTCAGCTGGTACA | |
| R3044_ | GCTGGGGACCGATCCTGATTGCTCGCTGCGGCGAGA | 610 |
| CasPhi32 | CTCACTGGATTTAGAGTCTCT | |
| R3045_ | GCTGGGGACCGATCCTGATTGCTCGCTGCGGCGAGA | 611 |
| CasPhi32 | CAGAATCAAAATCGGTGAATA | |
| R3046_ | GCTGGGGACCGATCCTGATTGCTCGCTGCGGCGAGA | 612 |
| CasPhi32 | CGAGAATCAAAATCGGTGAAT | |
| R3047_ | GCTGGGGACCGATCCTGATTGCTCGCTGCGGCGAGA | 613 |
| CasPhi32 | CACCGATTTTGATTCTCAAAC | |
| R3048_ | GCTGGGGACCGATCCTGATTGCTCGCTGCGGCGAGA | 614 |
| CasPhi32 | CTTTGAGAATCAAAATCGGTG | |
| R3049_ | GCTGGGGACCGATCCTGATTGCTCGCTGCGGCGAGA | 615 |
| CasPhi32 | CGTTTGAGAATCAAAATCGGT | |
| R3050_ | GCTGGGGACCGATCCTGATTGCTCGCTGCGGCGAGA | 616 |
| CasPhi32 | CTGATTCTCAAACAAATGTGT | |
| R3051_ | GCTGGGGACCGATCCTGATTGCTCGCTGCGGCGAGA | 617 |
| CasPhi32 | CGATTCTCAAACAAATGTGTC | |
| R3052_ | GCTGGGGACCGATCCTGATTGCTCGCTGCGGCGAGA | 618 |
| CasPhi32 | CATTCTCAAACAAATGTGTCA | |
| R3053_ | GCTGGGGACCGATCCTGATTGCTCGCTGCGGCGAGA | 619 |
| CasPhi32 | CTGACACATTTGTTTGAGAAT | |
| R3054_ | GCTGGGGACCGATCCTGATTGCTCGCTGCGGCGAGA | 620 |
| CasPhi32 | CTCAAACAAATGTGTCACAAA | |
| R3055_ | GCTGGGGACCGATCCTGATTGCTCGCTGCGGCGAGA | 621 |
| CasPhi32 | CGTGACACATTTGTTTGAGAA | |
| R3056_ | GCTGGGGACCGATCCTGATTGCTCGCTGCGGCGAGA | 622 |
| CasPhi32 | CCTTTGTGACACATTTGTTTG | |
| R3057_ | GCTGGGGACCGATCCTGATTGCTCGCTGCGGCGAGA | 623 |
| CasPhi32 | CTGATGTGTATATCACAGACA | |
| R3058_ | GCTGGGGACCGATCCTGATTGCTCGCTGCGGCGAGA | 624 |
| CasPhi32 | CTCTGTGATATACACATCAGA | |
| R3059_ | GCTGGGGACCGATCCTGATTGCTCGCTGCGGCGAGA | 625 |
| CasPhi32 | CGTCTGTGATATACACATCAG | |
| R3060_ | GCTGGGGACCGATCCTGATTGCTCGCTGCGGCGAGA | 626 |
| CasPhi32 | CTGTCTGTGATATACACATCA | |
| R3061_ | GCTGGGGACCGATCCTGATTGCTCGCTGCGGCGAGA | 627 |
| CasPhi32 | CAAGTCCATAGACCTCATGTC | |
| R3062_ | GCTGGGGACCGATCCTGATTGCTCGCTGCGGCGAGA | 628 |
| CasPhi32 | CCTCTTGAAGTCCATAGACCT | |
| R3063_ | GCTGGGGACCGATCCTGATTGCTCGCTGCGGCGAGA | 629 |
| CasPhi32 | CAAGAGCAACAGTGCTGTGGC | |
| R3064_ | GCTGGGGACCGATCCTGATTGCTCGCTGCGGCGAGA | 630 |
| CasPhi32 | CCTCCAGGCCACAGCACTGTT | |
| R3065_ | GCTGGGGACCGATCCTGATTGCTCGCTGCGGCGAGA | 631 |
| CasPhi32 | CTTGCTCCAGGCCACAGCACT | |
| R3066_ | GCTGGGGACCGATCCTGATTGCTCGCTGCGGCGAGA | 632 |
| CasPhi32 | CGTTGCTCCAGGCCACAGCAC | |
| R3067_ | GCTGGGGACCGATCCTGATTGCTCGCTGCGGCGAGA | 633 |
| CasPhi32 | CCACATGCAAAGTCAGATTTG | |
| R3068_ | GCTGGGGACCGATCCTGATTGCTCGCTGCGGCGAGA | 634 |
| CasPhi32 | CGCACATGCAAAGTCAGATTT | |
| R3069_ | GCTGGGGACCGATCCTGATTGCTCGCTGCGGCGAGA | 635 |
| CasPhi32 | CGCATGTGCAAACGCCTTCAA | |
| R3070_ | GCTGGGGACCGATCCTGATTGCTCGCTGCGGCGAGA | 636 |
| CasPhi32 | CAAGGCGTTTGCACATGCAAA | |
| R3071_ | GCTGGGGACCGATCCTGATTGCTCGCTGCGGCGAGA | 637 |
| CasPhi32 | CCATGTGCAAACGCCTTCAAC | |
| R3072_ | GCTGGGGACCGATCCTGATTGCTCGCTGCGGCGAGA | 638 |
| CasPhi32 | CTTGAAGGCGTTTGCACATGC | |
| R3073_ | GCTGGGGACCGATCCTGATTGCTCGCTGCGGCGAGA | 639 |
| CasPhi32 | CAACAACAGCATTATTCCAGA | |
| R3074_ | GCTGGGGACCGATCCTGATTGCTCGCTGCGGCGAGA | 640 |
| CasPhi32 | CTGGAATAATGCTGTTGTTGA | |
| R3075_ | GCTGGGGACCGATCCTGATTGCTCGCTGCGGCGAGA | 641 |
| CasPhi32 | CTTCCAGAAGACACCTTCTTC | |
| R3076_ | GCTGGGGACCGATCCTGATTGCTCGCTGCGGCGAGA | 642 |
| CasPhi32 | CCAGAAGACACCTTCTTCCCC | |
| R3077_ | GCTGGGGACCGATCCTGATTGCTCGCTGCGGCGAGA | 643 |
| CasPhi32 | CCCTGGGCTGGGGAAGAAGGT | |
| R3078_ | GCTGGGGACCGATCCTGATTGCTCGCTGCGGCGAGA | 644 |
| CasPhi32 | CTTCCCCAGCCCAGGTAAGGG | |
| R3079_ | GCTGGGGACCGATCCTGATTGCTCGCTGCGGCGAGA | 645 |
| CasPhi32 | CCCCAGCCCAGGTAAGGGCAG | |
| R3080_ | GCTGGGGACCGATCCTGATTGCTCGCTGCGGCGAGA | 646 |
| CasPhi32 | CTAAAAGGAAAAACAGACATT | |
| R3081_ | GCTGGGGACCGATCCTGATTGCTCGCTGCGGCGAGA | 647 |
| CasPhi32 | CCTAAAAGGAAAAACAGACAT | |
| R3082_ | GCTGGGGACCGATCCTGATTGCTCGCTGCGGCGAGA | 648 |
| CasPhi32 | CTTCCTTTTAGAAAGTTCCTG | |
| R3083_ | GCTGGGGACCGATCCTGATTGCTCGCTGCGGCGAGA | 649 |
| CasPhi32 | CTCCTTTTAGAAAGTTCCTGT | |
| R3084_ | GCTGGGGACCGATCCTGATTGCTCGCTGCGGCGAGA | 650 |
| CasPhi32 | CCCTTTTAGAAAGTTCCTGTG | |
| R3085_ | GCTGGGGACCGATCCTGATTGCTCGCTGCGGCGAGA | 651 |
| CasPhi32 | CCTTTTAGAAAGTTCCTGTGA | |
| R3086_ | GCTGGGGACCGATCCTGATTGCTCGCTGCGGCGAGA | 652 |
| CasPhi32 | CTAGAAAGTTCCTGTGATGTC | |
| R3136_ | GCTGGGGACCGATCCTGATTGCTCGCTGCGGCGAGA | 653 |
| CasPhi32 | CAGAAAGTTCCTGTGATGTCA | |
| R3137_ | GCTGGGGACCGATCCTGATTGCTCGCTGCGGCGAGA | 654 |
| CasPhi32 | CGAAAGTTCCTGTGATGTCAA | |
| R3138_ | GCTGGGGACCGATCCTGATTGCTCGCTGCGGCGAGA | 655 |
| CasPhi32 | CACATCACAGGAACTTTCTAA | |
| R3139_ | GCTGGGGACCGATCCTGATTGCTCGCTGCGGCGAGA | 656 |
| CasPhi32 | CCTGTGATGTCAAGCTGGTCG | |
| R3140_ | GCTGGGGACCGATCCTGATTGCTCGCTGCGGCGAGA | 657 |
| CasPhi32 | CTCGACCAGCTTGACATCACA | |
| R3141_ | GCTGGGGACCGATCCTGATTGCTCGCTGCGGCGAGA | 658 |
| CasPhi32 | CCTCGACCAGCTTGACATCAC | |
| R3142_ | GCTGGGGACCGATCCTGATTGCTCGCTGCGGCGAGA | 659 |
| CasPhi32 | CTCTCGACCAGCTTGACATCA | |
| R3143_ | GCTGGGGACCGATCCTGATTGCTCGCTGCGGCGAGA | 660 |
| CasPhi32 | CAAAGCTTTTCTCGACCAGCT | |
| R3144_ | GCTGGGGACCGATCCTGATTGCTCGCTGCGGCGAGA | 661 |
| CasPhi32 | CCAAAGCTTTTCTCGACCAGC | |
| R3145_ | GCTGGGGACCGATCCTGATTGCTCGCTGCGGCGAGA | 662 |
| CasPhi32 | CCCTGTTTCAAAGCTTTTCTC | |
| R3146_ | GCTGGGGACCGATCCTGATTGCTCGCTGCGGCGAGA | 663 |
| CasPhi32 | CGAAACAGGTAAGACAGGGGT | |
| R3147_ | GCTGGGGACCGATCCTGATTGCTCGCTGCGGCGAGA | 664 |
| CasPhi32 | CAAACAGGTAAGACAGGGGTC | |

TABLE K
CasΦ.12 gRNAs targeting human B2M in T cells

| Name | Repeat + spacer RNA Sequence (5′ → 3′), shown as DNA | SEQ ID NO |
|---|---|---|
| R3087_ | CTTTCAAGACTAATAGATTGCTCCTTACGAGGAGAC | 665 |
| CasPhi12 | AATATAAGTGGAGGCGTCGC | |
| R3088_ | CTTTCAAGACTAATAGATTGCTCCTTACGAGGAGAC | 666 |
| CasPhi12 | ATATAAGTGGAGGCGTCGCG | |
| R3089_ | CTTTCAAGACTAATAGATTGCTCCTTACGAGGAGAC | 667 |
| CasPhi12 | AGGAATGCCCGCCAGCGCGA | |
| R3090_ | CTTTCAAGACTAATAGATTGCTCCTTACGAGGAGAC | 668 |
| CasPhi12 | CTGAAGCTGACAGCATTCGG | |
| R3091_ | CTTTCAAGACTAATAGATTGCTCCTTACGAGGAGAC | 669 |
| CasPhi12 | GGGCCGAGATGTCTCGCTCC | |
| R3092_ | CTTTCAAGACTAATAGATTGCTCCTTACGAGGAGAC | 670 |
| CasPhi12 | GCTGTGCTCGCGCTACTCTC | |
| R3093_ | CTTTCAAGACTAATAGATTGCTCCTTACGAGGAGAC | 671 |
| CasPhi12 | CTGGCCTGGAGGCTATCCAG | |
| R3094_ | CTTTCAAGACTAATAGATTGCTCCTTACGAGGAGAC | 672 |
| CasPhi12 | TGGCCTGGAGGCTATCCAGC | |
| R3095_ | CTTTCAAGACTAATAGATTGCTCCTTACGAGGAGAC | 673 |
| CasPhi12 | ATGTGTCTTTTCCCGATATT | |
| R3096_ | CTTTCAAGACTAATAGATTGCTCCTTACGAGGAGAC | 674 |
| CasPhi12 | TCCCGATATTCCTCAGGTAC | |
| R3097_ | CTTTCAAGACTAATAGATTGCTCCTTACGAGGAGAC | 675 |
| CasPhi12 | CCCGATATTCCTCAGGTACT | |
| R3098_ | CTTTCAAGACTAATAGATTGCTCCTTACGAGGAGAC | 676 |
| CasPhi12 | CCGATATTCCTCAGGTACTC | |
| R3099_ | CTTTCAAGACTAATAGATTGCTCCTTACGAGGAGAC | 677 |
| CasPhi12 | GAGTACCTGAGGAATATCGG | |
| R3100_ | CTTTCAAGACTAATAGATTGCTCCTTACGAGGAGAC | 678 |
| CasPhi12 | GGAGTACCTGAGGAATATCG | |
| R3101_ | CTTTCAAGACTAATAGATTGCTCCTTACGAGGAGAC | 679 |
| CasPhi12 | CTCAGGTACTCCAAAGATTC | |
| R3102_ | CTTTCAAGACTAATAGATTGCTCCTTACGAGGAGAC | 680 |
| CasPhi12 | AGGTTTACTCACGTCATCCA | |
| R3103_ | CTTTCAAGACTAATAGATTGCTCCTTACGAGGAGAC | 681 |
| CasPhi12 | ACTCACGTCATCCAGCAGAG | |
| R3104_ | CTTTCAAGACTAATAGATTGCTCCTTACGAGGAGAC | 682 |
| CasPhi12 | CTCACGTCATCCAGCAGAGA | |
| R3105_ | CTTTCAAGACTAATAGATTGCTCCTTACGAGGAGAC | 683 |
| CasPhi12 | TCTGCTGGATGACGTGAGTA | |
| R3106_ | CTTTCAAGACTAATAGATTGCTCCTTACGAGGAGAC | 684 |
| CasPhi12 | CATTCTCTGCTGGATGACGT | |
| R3107_ | CTTTCAAGACTAATAGATTGCTCCTTACGAGGAGAC | 685 |
| CasPhi12 | CCATTCTCTGCTGGATGACG | |
| R3108_ | CTTTCAAGACTAATAGATTGCTCCTTACGAGGAGAC | 686 |
| CasPhi12 | ACTTTCCATTCTCTGCTGGA | |
| R3109_ | CTTTCAAGACTAATAGATTGCTCCTTACGAGGAGAC | 687 |
| CasPhi12 | GACTTTCCATTCTCTGCTGG | |
| R3110_ | CTTTCAAGACTAATAGATTGCTCCTTACGAGGAGAC | 688 |
| CasPhi12 | AGGAAATTTGACTTTCCATT | |
| R3111_ | CTTTCAAGACTAATAGATTGCTCCTTACGAGGAGAC | 689 |
| CasPhi12 | CCTGAATTGCTATGTGTCTG | |
| R3112_ | CTTTCAAGACTAATAGATTGCTCCTTACGAGGAGAC | 690 |
| CasPhi12 | CTGAATTGCTATGTGTCTGG | |
| R3113_ | CTTTCAAGACTAATAGATTGCTCCTTACGAGGAGAC | 691 |
| CasPhi12 | CTATGTGTCTGGGTTTCATC | |
| R3114_ | CTTTCAAGACTAATAGATTGCTCCTTACGAGGAGAC | 692 |
| CasPhi12 | AATGTCGGATGGATGAAACC | |
| R3115_ | CTTTCAAGACTAATAGATTGCTCCTTACGAGGAGAC | 693 |
| CasPhi12 | CATCCATCCGACATTGAAGT | |
| R3116_ | CTTTCAAGACTAATAGATTGCTCCTTACGAGGAGAC | 694 |
| CasPhi12 | ATCCATCCGACATTGAAGTT | |
| R3117_ | CTTTCAAGACTAATAGATTGCTCCTTACGAGGAGAC | 695 |
| CasPhi12 | AGTAAGTCAACTTCAATGTC | |
| R3118_ | CTTTCAAGACTAATAGATTGCTCCTTACGAGGAGAC | 696 |
| CasPhi12 | TTCAGTAAGTCAACTTCAAT | |
| R3119_ | CTTTCAAGACTAATAGATTGCTCCTTACGAGGAGAC | 697 |
| CasPhi12 | AAGTTGACTTACTGAAGAAT | |
| R3120_ | CTTTCAAGACTAATAGATTGCTCCTTACGAGGAGAC | 698 |
| CasPhi12 | ACTTACTGAAGAATGGAGAG | |
| R3121_ | CTTTCAAGACTAATAGATTGCTCCTTACGAGGAGAC | 699 |
| CasPhi12 | TCTCTCCATTCTTCAGTAAG | |
| R3122_ | CTTTCAAGACTAATAGATTGCTCCTTACGAGGAGAC | 700 |
| CasPhi12 | CTGAAGAATGGAGAGAGAAT | |
| R3123_ | CTTTCAAGACTAATAGATTGCTCCTTACGAGGAGAC | 701 |
| CasPhi12 | AATTCTCTCTCCATTCTTCA | |
| R3124_ | CTTTCAAGACTAATAGATTGCTCCTTACGAGGAGAC | 702 |
| CasPhi12 | CAATTCTCTCTCCATTCTTC | |
| R3125_ | CTTTCAAGACTAATAGATTGCTCCTTACGAGGAGAC | 703 |
| CasPhi12 | TCAATTCTCTCTCCATTCTT | |
| R3126_ | CTTTCAAGACTAATAGATTGCTCCTTACGAGGAGAC | 704 |
| CasPhi12 | TTCAATTCTCTCTCCATTCT | |
| R3127_ | CTTTCAAGACTAATAGATTGCTCCTTACGAGGAGAC | 705 |
| CasPhi12 | AAAAAGTGGAGCATTCAGAC | |
| R3128_ | CTTTCAAGACTAATAGATTGCTCCTTACGAGGAGAC | 706 |
| CasPhi12 | CTGAAAGACAAGTCTGAATG | |
| R3129_ | CTTTCAAGACTAATAGATTGCTCCTTACGAGGAGAC | 707 |
| CasPhi12 | AGACTTGTCTTTCAGCAAGG | |
| R3130_ | CTTTCAAGACTAATAGATTGCTCCTTACGAGGAGAC | 708 |
| CasPhi12 | TCTTTCAGCAAGGACTGGTC | |
| R3131_ | CTTTCAAGACTAATAGATTGCTCCTTACGAGGAGAC | 709 |
| CasPhi12 | CAGCAAGGACTGGTCTTTCT | |
| R3132_ | CTTTCAAGACTAATAGATTGCTCCTTACGAGGAGAC | 710 |
| CasPhi12 | AGCAAGGACTGGTCTTTCTA | |
| R3133_ | CTTTCAAGACTAATAGATTGCTCCTTACGAGGAGAC | 711 |
| CasPhi12 | CTATCTCTTGTACTACACTG | |
| R3134_ | CTTTCAAGACTAATAGATTGCTCCTTACGAGGAGAC | 712 |
| CasPhi12 | TATCTCTTGTACTACACTGA | |
| R3135_ | CTTTCAAGACTAATAGATTGCTCCTTACGAGGAGAC | 713 |
| CasPhi12 | AGTGTAGTACAAGAGATAGA | |
| R3148_ | CTTTCAAGACTAATAGATTGCTCCTTACGAGGAGAC | 714 |
| CasPhi12 | TACTACACTGAATTCACCCC | |
| R3149_ | CTTTCAAGACTAATAGATTGCTCCTTACGAGGAGAC | 715 |
| CasPhi12 | AGTGGGGGTGAATTCAGTGT | |
| R3150_ | CTTTCAAGACTAATAGATTGCTCCTTACGAGGAGAC | 716 |
| CasPhi12 | CAGTGGGGGTGAATTCAGTG | |
| R3151_ | CTTTCAAGACTAATAGATTGCTCCTTACGAGGAGAC | 717 |
| CasPhi12 | TCAGTGGGGGTGAATTCAGT | |
| R3152_ | CTTTCAAGACTAATAGATTGCTCCTTACGAGGAGAC | 718 |
| CasPhi12 | TTCAGTGGGGGTGAATTCAG | |
| R3153_ | CTTTCAAGACTAATAGATTGCTCCTTACGAGGAGAC | 719 |
| CasPhi12 | ACCCCCACTGAAAAAGATGA | |
| R3154_ | CTTTCAAGACTAATAGATTGCTCCTTACGAGGAGAC | 720 |
| CasPhi12 | ACACGGCAGGCATACTCATC | |
| R3155_ | CTTTCAAGACTAATAGATTGCTCCTTACGAGGAGAC | 721 |
| CasPhi12 | GGCTGTGACAAAGTCACATG | |
| R3156_ | CTTTCAAGACTAATAGATTGCTCCTTACGAGGAGAC | 722 |
| CasPhi12 | GTCACAGCCCAAGATAGTTA | |
| R3157_ | CTTTCAAGACTAATAGATTGCTCCTTACGAGGAGAC | 723 |
| CasPhi12 | TCACAGCCCAAGATAGTTAA | |
| R3158_ | CTTTCAAGACTAATAGATTGCTCCTTACGAGGAGAC | 724 |
| CasPhi12 | ACTATCTTGGGCTGTGACAA | |
| R3159_ | CTTTCAAGACTAATAGATTGCTCCTTACGAGGAGAC | 725 |
| CasPhi12 | CCCCACTTAACTATCTTGGG | |

TABLE L
CasΦ.32 gRNAs targeting human B2M in T cells

| Name | Repeat + spacer RNA Sequence (5′ → 3′), shown as DNA | SEQ ID NO |
|---|---|---|
| R3087_ | GCTGGGGACCGATCCTGATTGCTCGCTGCGGCGAGA | 726 |
| CasPhi32 | CAATATAAGTGGAGGCGTCGC | |
| R3088_ | GCTGGGGACCGATCCTGATTGCTCGCTGCGGCGAGA | 727 |
| CasPhi32 | CATATAAGTGGAGGCGTCGCG | |
| R3089_ | GCTGGGGACCGATCCTGATTGCTCGCTGCGGCGAGA | 728 |
| CasPhi32 | CAGGAATGCCCGCCAGCGCGA | |
| R3090_ | GCTGGGGACCGATCCTGATTGCTCGCTGCGGCGAGA | 729 |
| CasPhi32 | CCTGAAGCTGACAGCATTCGG | |
| R3091_ | GCTGGGGACCGATCCTGATTGCTCGCTGCGGCGAGA | 730 |
| CasPhi32 | CGGGCCGAGATGTCTCGCTCC | |
| R3092_ | GCTGGGGACCGATCCTGATTGCTCGCTGCGGCGAGA | 731 |
| CasPhi32 | CGCTGTGCTCGCGCTACTCTC | |
| R3093_ | GCTGGGGACCGATCCTGATTGCTCGCTGCGGCGAGA | 732 |
| CasPhi32 | CCTGGCCTGGAGGCTATCCAG | |
| R3094_ | GCTGGGGACCGATCCTGATTGCTCGCTGCGGCGAGA | 733 |
| CasPhi32 | CTGGCCTGGAGGCTATCCAGC | |
| R3095_ | GCTGGGGACCGATCCTGATTGCTCGCTGCGGCGAGA | 734 |
| CasPhi32 | CATGTGTCTTTTCCCGATATT | |
| R3096_ | GCTGGGGACCGATCCTGATTGCTCGCTGCGGCGAGA | 735 |
| CasPhi32 | CTCCCGATATTCCTCAGGTAC | |
| R3097_ | GCTGGGGACCGATCCTGATTGCTCGCTGCGGCGAGA | 736 |
| CasPhi32 | CCCCGATATTCCTCAGGTACT | |
| R3098_ | GCTGGGGACCGATCCTGATTGCTCGCTGCGGCGAGA | 737 |
| CasPhi32 | CCCGATATTCCTCAGGTACTC | |
| R3099_ | GCTGGGGACCGATCCTGATTGCTCGCTGCGGCGAGA | 738 |
| CasPhi32 | CGAGTACCTGAGGAATATCGG | |
| R3100_ | GCTGGGGACCGATCCTGATTGCTCGCTGCGGCGAGA | 739 |
| CasPhi32 | CGGAGTACCTGAGGAATATCG | |
| R3101_ | GCTGGGGACCGATCCTGATTGCTCGCTGCGGCGAGA | 740 |
| CasPhi32 | CCTCAGGTACTCCAAAGATTC | |
| R3102_ | GCTGGGGACCGATCCTGATTGCTCGCTGCGGCGAGA | 741 |
| CasPhi32 | CAGGTTTACTCACGTCATCCA | |
| R3103_ | GCTGGGGACCGATCCTGATTGCTCGCTGCGGCGAGA | 742 |
| CasPhi32 | CACTCACGTCATCCAGCAGAG | |
| R3104_ | GCTGGGGACCGATCCTGATTGCTCGCTGCGGCGAGA | 743 |
| CasPhi32 | CCTCACGTCATCCAGCAGAGA | |
| R3105_ | GCTGGGGACCGATCCTGATTGCTCGCTGCGGCGAGA | 744 |
| CasPhi32 | CTCTGCTGGATGACGTGAGTA | |
| R3106_ | GCTGGGGACCGATCCTGATTGCTCGCTGCGGCGAGA | 745 |
| CasPhi32 | CCATTCTCTGCTGGATGACGT | |
| R3107_ | GCTGGGGACCGATCCTGATTGCTCGCTGCGGCGAGA | 746 |
| CasPhi32 | CCCATTCTCTGCTGGATGACG | |
| R3108_ | GCTGGGGACCGATCCTGATTGCTCGCTGCGGCGAGA | 747 |
| CasPhi32 | CACTTTCCATTCTCTGCTGGA | |
| R3109_ | GCTGGGGACCGATCCTGATTGCTCGCTGCGGCGAGA | 748 |
| CasPhi32 | CGACTTTCCATTCTCTGCTGG | |
| R3110_ | GCTGGGGACCGATCCTGATTGCTCGCTGCGGCGAGA | 749 |
| CasPhi32 | CAGGAAATTTGACTTTCCATT | |
| R3111_ | GCTGGGGACCGATCCTGATTGCTCGCTGCGGCGAGA | 750 |
| CasPhi32 | CCCTGAATTGCTATGTGTCTG | |
| R3112_ | GCTGGGGACCGATCCTGATTGCTCGCTGCGGCGAGA | 751 |
| CasPhi32 | CCTGAATTGCTATGTGTCTGG | |
| R3113_ | GCTGGGGACCGATCCTGATTGCTCGCTGCGGCGAGA | 752 |
| CasPhi32 | CCTATGTGTCTGGGTTTCATC | |
| R3114_ | GCTGGGGACCGATCCTGATTGCTCGCTGCGGCGAGA | 753 |
| CasPhi32 | CAATGTCGGATGGATGAAACC | |
| R3115_ | GCTGGGGACCGATCCTGATTGCTCGCTGCGGCGAGA | 754 |
| CasPhi32 | CCATCCATCCGACATTGAAGT | |
| R3116_ | GCTGGGGACCGATCCTGATTGCTCGCTGCGGCGAGA | 755 |
| CasPhi32 | CATCCATCCGACATTGAAGTT | |
| R3117_ | GCTGGGGACCGATCCTGATTGCTCGCTGCGGCGAGA | 756 |
| CasPhi32 | CAGTAAGTCAACTTCAATGTC | |
| R3118_ | GCTGGGGACCGATCCTGATTGCTCGCTGCGGCGAGA | 757 |
| CasPhi32 | CTTCAGTAAGTCAACTTCAAT | |
| R3119_ | GCTGGGGACCGATCCTGATTGCTCGCTGCGGCGAGA | 758 |
| CasPhi32 | CAAGTTGACTTACTGAAGAAT | |
| R3120_ | GCTGGGGACCGATCCTGATTGCTCGCTGCGGCGAGA | 759 |
| CasPhi32 | CACTTACTGAAGAATGGAGAG | |
| R3121_ | GCTGGGGACCGATCCTGATTGCTCGCTGCGGCGAGA | 760 |
| CasPhi32 | CTCTCTCCATTCTTCAGTAAG | |
| R3122_ | GCTGGGGACCGATCCTGATTGCTCGCTGCGGCGAGA | 761 |
| CasPhi32 | CCTGAAGAATGGAGAGAGAAT | |
| R3123_ | GCTGGGGACCGATCCTGATTGCTCGCTGCGGCGAGA | 762 |
| CasPhi32 | CAATTCTCTCTCCATTCTTCA | |
| R3124_ | GCTGGGGACCGATCCTGATTGCTCGCTGCGGCGAGA | 763 |
| CasPhi32 | CCAATTCTCTCTCCATTCTTC | |
| R3125_ | GCTGGGGACCGATCCTGATTGCTCGCTGCGGCGAGA | 764 |
| CasPhi32 | CTCAATTCTCTCTCCATTCTT | |
| R3126_ | GCTGGGGACCGATCCTGATTGCTCGCTGCGGCGAGA | 765 |
| CasPhi32 | CTTCAATTCTCTCTCCATTCT | |
| R3127_ | GCTGGGGACCGATCCTGATTGCTCGCTGCGGCGAGA | 766 |
| CasPhi32 | CAAAAAGTGGAGCATTCAGAC | |
| R3128_ | GCTGGGGACCGATCCTGATTGCTCGCTGCGGCGAGA | 767 |
| CasPhi32 | CCTGAAAGACAAGTCTGAATG | |
| R3129_ | GCTGGGGACCGATCCTGATTGCTCGCTGCGGCGAGA | 768 |
| CasPhi32 | CAGACTTGTCTTTCAGCAAGG | |
| R3130_ | GCTGGGGACCGATCCTGATTGCTCGCTGCGGCGAGA | 769 |
| CasPhi32 | CTCTTTCAGCAAGGACTGGTC | |
| R3131_ | GCTGGGGACCGATCCTGATTGCTCGCTGCGGCGAGA | 770 |
| CasPhi32 | CCAGCAAGGACTGGTCTTTCT | |
| R3132_ | GCTGGGGACCGATCCTGATTGCTCGCTGCGGCGAGA | 771 |
| CasPhi32 | CAGCAAGGACTGGTCTTTCTA | |
| R3133_ | GCTGGGGACCGATCCTGATTGCTCGCTGCGGCGAGA | 772 |
| CasPhi32 | CCTATCTCTTGTACTACACTG | |
| R3134_ | GCTGGGGACCGATCCTGATTGCTCGCTGCGGCGAGA | 773 |
| CasPhi32 | CTATCTCTTGTACTACACTGA | |
| R3135_ | GCTGGGGACCGATCCTGATTGCTCGCTGCGGCGAGA | 774 |
| CasPhi32 | CAGTGTAGTACAAGAGATAGA | |
| R3148_ | GCTGGGGACCGATCCTGATTGCTCGCTGCGGCGAGA | 775 |
| CasPhi32 | CTACTACACTGAATTCACCCC | |
| R3149_ | GCTGGGGACCGATCCTGATTGCTCGCTGCGGCGAGA | 776 |
| CasPhi32 | CAGTGGGGGTGAATTCAGTGT | |
| R3150_ | GCTGGGGACCGATCCTGATTGCTCGCTGCGGCGAGA | 777 |
| CasPhi32 | CCAGTGGGGGTGAATTCAGTG | |
| R3151_ | GCTGGGGACCGATCCTGATTGCTCGCTGCGGCGAGA | 778 |
| CasPhi32 | CTCAGTGGGGGTGAATTCAGT | |
| R3152_ | GCTGGGGACCGATCCTGATTGCTCGCTGCGGCGAGA | 779 |
| CasPhi32 | CTTCAGTGGGGGTGAATTCAG | |
| R3153_ | GCTGGGGACCGATCCTGATTGCTCGCTGCGGCGAGA | 780 |
| CasPhi32 | CACCCCCACTGAAAAAGATGA | |
| R3154_ | GCTGGGGACCGATCCTGATTGCTCGCTGCGGCGAGA | 781 |
| CasPhi32 | CACACGGCAGGCATACTCATC | |
| R3155_ | GCTGGGGACCGATCCTGATTGCTCGCTGCGGCGAGA | 782 |
| CasPhi32 | CGGCTGTGACAAAGTCACATG | |
| R3156_ | GCTGGGGACCGATCCTGATTGCTCGCTGCGGCGAGA | 783 |
| CasPhi32 | CGTCACAGCCCAAGATAGTTA | |
| R3157_ | GCTGGGGACCGATCCTGATTGCTCGCTGCGGCGAGA | 784 |
| CasPhi32 | CTCACAGCCCAAGATAGTTAA | |
| R3158_ | GCTGGGGACCGATCCTGATTGCTCGCTGCGGCGAGA | 785 |
| CasPhi32 | CACTATCTTGGGCTGTGACAA | |
| R3159_ | GCTGGGGACCGATCCTGATTGCTCGCTGCGGCGAGA | 786 |
| CasPhi32 | CCCCCACTTAACTATCTTGGG | |

TABLE M
CasΦ.12 gRNAs targeting human PD1 in T cells

| Name | Repeat + spacer RNA Sequence (5′ → 3′) | SEQ ID NO |
|---|---|---|
| R2921_ | CUUUCAAGACUAAUAGAUUGCUCCUUACGAGGAG | 787 |
| CasPhi12 | ACCCUUCCGCUCACCUCCGCCU | |
| R2922_ | CUUUCAAGACUAAUAGAUUGCUCCUUACGAGGAG | 788 |
| CasPhi12 | ACCCUUCCGCUCACCUCCGCCU | |
| R2923_ | CUUUCAAGACUAAUAGAUUGCUCCUUACGAGGAG | 789 |
| CasPhi12 | ACCGCUCACCUCCGCCUGAGCA | |
| R2924_ | CUUUCAAGACUAAUAGAUUGCUCCUUACGAGGAG | 790 |
| CasPhi12 | ACUCCACUGCUCAGGCGGAGGU | |
| R2925_ | CUUUCAAGACUAAUAGAUUGCUCCUUACGAGGAG | 791 |
| CasPhi12 | ACUAGCACCGCCCAGACGACUG | |
| R2926_ | CUUUCAAGACUAAUAGAUUGCUCCUUACGAGGAG | 792 |
| CasPhi12 | ACAGGCAUGCAGAUCCCACAGG | |
| R2927_ | CUUUCAAGACUAAUAGAUUGCUCCUUACGAGGAG | 793 |
| CasPhi12 | ACCACAGGCGCCCUGGCCAGUC | |
| R2928_ | CUUUCAAGACUAAUAGAUUGCUCCUUACGAGGAG | 794 |
| CasPhi12 | ACUCUGGGCGGUGCUACAACUG | |
| R2929_ | CUUUCAAGACUAAUAGAUUGCUCCUUACGAGGAG | 795 |
| CasPhi12 | ACGCAUGCCUGGAGCAGCCCCA | |
| R2930_ | CUUUCAAGACUAAUAGAUUGCUCCUUACGAGGAG | 796 |
| CasPhi12 | ACUAGCACCGCCCAGACGACUG | |
| R2931_ | CUUUCAAGACUAAUAGAUUGCUCCUUACGAGGAG | 797 |
| CasPhi12 | ACUGGCCGCCAGCCCAGUUGUA | |
| R2932_ | CUUUCAAGACUAAUAGAUUGCUCCUUACGAGGAG | 798 |
| CasPhi12 | ACCUUCCGCUCACCUCCGCCUG | |
| R2933_ | CUUUCAAGACUAAUAGAUUGCUCCUUACGAGGAG | 799 |
| CasPhi12 | ACCAGGGCCUGUCUGGGGAGUC | |
| R2934_ | CUUUCAAGACUAAUAGAUUGCUCCUUACGAGGAG | 800 |
| CasPhi12 | ACUCCCCAGCCCUGCUCGUGGU | |
| R2935_ | CUUUCAAGACUAAUAGAUUGCUCCUUACGAGGAG | 801 |
| CasPhi12 | ACGGUCACCACGAGCAGGGCUG | |
| R2936_ | CUUUCAAGACUAAUAGAUUGCUCCUUACGAGGAG | 802 |
| CasPhi12 | ACUCCCCUUCGGUCACCACGAG | |
| R2937_ | CUUUCAAGACUAAUAGAUUGCUCCUUACGAGGAG | 803 |
| CasPhi12 | ACGAGAAGCUGCAGGUGAAGGU | |
| R2938_ | CUUUCAAGACUAAUAGAUUGCUCCUUACGAGGAG | 804 |
| CasPhi12 | ACACCUGCAGCUUCUCCAACAC | |
| R2939_ | CUUUCAAGACUAAUAGAUUGCUCCUUACGAGGAG | 805 |
| CasPhi12 | ACUCCAACACAUCGGAGAGCUU | |
| R2940_ | CUUUCAAGACUAAUAGAUUGCUCCUUACGAGGAG | 806 |
| CasPhi12 | ACGCACGAAGCUCUCCGAUGUG | |
| R2941_ | CUUUCAAGACUAAUAGAUUGCUCCUUACGAGGAG | 807 |
| CasPhi12 | ACAGCACGAAGCUCUCCGAUGU | |
| R2942_ | CUUUCAAGACUAAUAGAUUGCUCCUUACGAGGAG | 808 |
| CasPhi12 | ACGUGCUAAACUGGUACCGCAU | |
| R2943_ | CUUUCAAGACUAAUAGAUUGCUCCUUACGAGGAG | 809 |
| CasPhi12 | ACCUGGGGCUCAUGCGGUACCA | |
| R2944_ | CUUUCAAGACUAAUAGAUUGCUCCUUACGAGGAG | 810 |
| CasPhi12 | ACUCCGUCUGGUUGCUGGGGCU | |
| R2945_ | CUUUCAAGACUAAUAGAUUGCUCCUUACGAGGAG | 811 |
| CasPhi12 | ACCCCGAGGACCGCAGCCAGCC | |
| R2946_ | CUUUCAAGACUAAUAGAUUGCUCCUUACGAGGAG | 812 |
| CasPhi12 | ACUGUGACACGGAAGCGGCAGU | |
| R2947_ | CUUUCAAGACUAAUAGAUUGCUCCUUACGAGGAG | 813 |
| CasPhi12 | ACCGUGUCACACAACUGCCCAA | |
| R2948_ | CUUUCAAGACUAAUAGAUUGCUCCUUACGAGGAG | 814 |
| CasPhi12 | ACGGCAGUUGUGUGACACGGAA | |
| R2949_ | CUUUCAAGACUAAUAGAUUGCUCCUUACGAGGAG | 815 |
| CasPhi12 | ACCACAUGAGCGUGGUCAGGGC | |
| R2950_ | CUUUCAAGACUAAUAGAUUGCUCCUUACGAGGAG | 816 |
| CasPhi12 | ACCGCCGGGCCCUGACCACGCU | |
| R2951_ | CUUUCAAGACUAAUAGAUUGCUCCUUACGAGGAG | 817 |
| CasPhi12 | ACGGGGCCAGGGAGAUGGCCCC | |
| R2952_ | CUUUCAAGACUAAUAGAUUGCUCCUUACGAGGAG | 818 |
| CasPhi12 | ACAUCUGCGCCUUGGGGGCCAG | |
| R2953_ | CUUUCAAGACUAAUAGAUUGCUCCUUACGAGGAG | 819 |
| CasPhi12 | ACGAUCUGCGCCUUGGGGGCCA | |
| R2954_ | CUUUCAAGACUAAUAGAUUGCUCCUUACGAGGAG | 820 |
| CasPhi12 | ACCCAGACAGGCCCUGGAACCC | |
| R2955_ | CUUUCAAGACUAAUAGAUUGCUCCUUACGAGGAG | 821 |
| CasPhi12 | ACCCAGCCCUGCUCGUGGUGAC | |
| R2956_ | CUUUCAAGACUAAUAGAUUGCUCCUUACGAGGAG | 822 |
| CasPhi12 | ACUCUCUGGAAGGGCACAAAGG | |
| R2957_ | CUUUCAAGACUAAUAGAUUGCUCCUUACGAGGAG | 823 |
| CasPhi12 | ACGUGCCCUUCCAGAGAGAAGG | |
| R2958_ | CUUUCAAGACUAAUAGAUUGCUCCUUACGAGGAG | 824 |
| CasPhi12 | ACUGCCCUUCCAGAGAGAAGGG | |
| R2959_ | CUUUCAAGACUAAUAGAUUGCUCCUUACGAGGAG | 825 |
| CasPhi12 | ACUGCCCUUCUCUCUGGAAGGG | |
| R2960_ | CUUUCAAGACUAAUAGAUUGCUCCUUACGAGGAG | 826 |
| CasPhi12 | ACCAGAGAGAAGGGCAGAAGUG | |
| R2961_ | CUUUCAAGACUAAUAGAUUGCUCCUUACGAGGAG | 827 |
| CasPhi12 | ACGAACUGGCCGGCUGGCCUGG | |
| R2962_ | CUUUCAAGACUAAUAGAUUGCUCCUUACGAGGAG | 828 |
| CasPhi12 | ACGGAACUGGCCGGCUGGCCUG | |
| R2963_ | CUUUCAAGACUAAUAGAUUGCUCCUUACGAGGAG | 829 |
| CasPhi12 | ACCAAACCCUGGUGGUUGGUGU | |
| R2964_ | CUUUCAAGACUAAUAGAUUGCUCCUUACGAGGAG | 830 |
| CasPhi12 | ACGUGUCGUGGGCGGCCUGCUG | |
| R2965_ | CUUUCAAGACUAAUAGAUUGCUCCUUACGAGGAG | 831 |
| CasPhi12 | ACCCUCGUGCGGCCCGGGAGCA | |
| R2966_ | CUUUCAAGACUAAUAGAUUGCUCCUUACGAGGAG | 832 |
| CasPhi12 | ACUCCCUGCAGAGAAACACACU | |
| R2967_ | CUUUCAAGACUAAUAGAUUGCUCCUUACGAGGAG | 833 |
| CasPhi12 | ACCUCUGCAGGGACAAUAGGAG | |
| R2968_ | CUUUCAAGACUAAUAGAUUGCUCCUUACGAGGAG | 834 |
| CasPhi12 | ACUCUGCAGGGACAAUAGGAGC | |
| R2969_ | CUUUCAAGACUAAUAGAUUGCUCCUUACGAGGAG | 835 |
| CasPhi12 | ACCUCCUCAAAGAAGGAGGACC | |
| R2970_ | CUUUCAAGACUAAUAGAUUGCUCCUUACGAGGAG | 836 |
| CasPhi12 | ACUCCUCAAAGAAGGAGGACCC | |
| R2971_ | CUUUCAAGACUAAUAGAUUGCUCCUUACGAGGAG | 837 |
| CasPhi12 | ACUCUGUGGACUAUGGGGAGCU | |
| R2972_ | CUUUCAAGACUAAUAGAUUGCUCCUUACGAGGAG | 838 |
| CasPhi12 | ACUCUCGCCACUGGAAAUCCAG | |
| R2973_ | CUUUCAAGACUAAUAGAUUGCUCCUUACGAGGAG | 839 |
| CasPhi12 | ACCCAGUGGCGAGAGAAGACCC | |
| R2974_ | CUUUCAAGACUAAUAGAUUGCUCCUUACGAGGAG | 840 |
| CasPhi12 | ACCAGUGGCGAGAGAAGACCCC | |
| R2975_ | CUUUCAAGACUAAUAGAUUGCUCCUUACGAGGAG | 841 |
| CasPhi12 | ACCGCUAGGAAAGACAAUGGUG | |
| R2976_ | CUUUCAAGACUAAUAGAUUGCUCCUUACGAGGAG | 842 |
| CasPhi12 | ACUCUUUCCUAGCGGAAUGGGC | |
| R2977_ | CUUUCAAGACUAAUAGAUUGCUCCUUACGAGGAG | 843 |
| CasPhi12 | ACCCUAGCGGAAUGGGCACCUC | |
| R2978_ | CUUUCAAGACUAAUAGAUUGCUCCUUACGAGGAG | 844 |
| CasPhi12 | ACCUAGCGGAAUGGGCACCUCA | |
| R2979_ | CUUUCAAGACUAAUAGAUUGCUCCUUACGAGGAG | 845 |
| CasPhi12 | ACGCCCCUCUGACCGGCUUCCU | |
| R2980_ | CUUUCAAGACUAAUAGAUUGCUCCUUACGAGGAG | 846 |
| CasPhi12 | ACCUUGGCCACCAGUGUUCUGC | |
| R2981_ | CUUUCAAGACUAAUAGAUUGCUCCUUACGAGGAG | 847 |
| CasPhi12 | ACGCCACCAGUGUUCUGCAGAC | |
| R2982_ | CUUUCAAGACUAAUAGAUUGCUCCUUACGAGGAG | 848 |
| CasPhi12 | ACUGCAGACCCUCCACCAUGAG | |
| R2983_ | CUUUCAAGACUAAUAGAUUGCUCCUUACGAGGAG | 849 |
| CasPhi12 | ACUCCUGAGGAAAUGCGCUGAC | |
| R2984_ | CUUUCAAGACUAAUAGAUUGCUCCUUACGAGGAG | 850 |
| CasPhi12 | ACCCUCAGGAGAAGCAGGCAGG | |
| R2985_ | CUUUCAAGACUAAUAGAUUGCUCCUUACGAGGAG | 851 |
| CasPhi12 | ACCUCAGGAGAAGCAGGCAGGG | |
| R2986_ | CUUUCAAGACUAAUAGAUUGCUCCUUACGAGGAG | 852 |
| CasPhi12 | ACCAGGCCGUCCAGGGGCUGAG | |
| R2987_ | CUUUCAAGACUAAUAGAUUGCUCCUUACGAGGAG | 853 |
| CasPhi12 | ACAGACAUGAGUCCUGUGGUGG | |
| R2988_ | CUUUCAAGACUAAUAGAUUGCUCCUUACGAGGAG | 854 |
| CasPhi12 | ACAGGUCCUGCCAGCACAGAGC | |
| R2989_ | CUUUCAAGACUAAUAGAUUGCUCCUUACGAGGAG | 855 |
| CasPhi12 | ACAGGGAGCUGGACGCAGGCAG | |
| R2990_ | CUUUCAAGACUAAUAGAUUGCUCCUUACGAGGAG | 856 |
| CasPhi12 | ACAGCCCCGGGCCGCAGGCAGC | |
| R2991_ | CUUUCAAGACUAAUAGAUUGCUCCUUACGAGGAG | 857 |
| CasPhi12 | ACAGGCAGGAGGCUCCGGGGCG | |
| R2992_ | CUUUCAAGACUAAUAGAUUGCUCCUUACGAGGAG | 858 |
| CasPhi12 | ACGGGGCUGGUUGGAGAUGGCC | |
| R2993_ | CUUUCAAGACUAAUAGAUUGCUCCUUACGAGGAG | 859 |
| CasPhi12 | ACGAGAUGGCCUUGGAGCAGCC | |
| R2994_ | CUUUCAAGACUAAUAGAUUGCUCCUUACGAGGAG | 860 |
| CasPhi12 | ACGCUGCUCCAAGGCCAUCUCC | |
| R2995_ | CUUUCAAGACUAAUAGAUUGCUCCUUACGAGGAG | 861 |
| CasPhi12 | ACGAGCAGCCAAGGUGCCCCUG | |
| R2996_ | CUUUCAAGACUAAUAGAUUGCUCCUUACGAGGAG | 862 |
| CasPhi12 | ACGGGAUGCCACUGCCAGGGGC | |
| R2997_ | CUUUCAAGACUAAUAGAUUGCUCCUUACGAGGAG | 863 |
| CasPhi12 | ACCGGGAUGCCACUGCCAGGGG | |
| R2998_ | CUUUCAAGACUAAUAGAUUGCUCCUUACGAGGAG | 864 |
| CasPhi12 | ACGGCCCUGCGUCCAGGGCGUU | |
| R2999_ | CUUUCAAGACUAAUAGAUUGCUCCUUACGAGGAG | 865 |
| CasPhi12 | ACUCUGCUCCCUGCAGGCCUAG | |
| R3000_ | CUUUCAAGACUAAUAGAUUGCUCCUUACGAGGAG | 866 |
| CasPhi12 | ACUCUAGGCCUGCAGGGAGCAG | |
| R3001_ | CUUUCAAGACUAAUAGAUUGCUCCUUACGAGGAG | 867 |
| CasPhi12 | ACCCUGAAACUUCUCUAGGCCU | |
| R3002_ | CUUUCAAGACUAAUAGAUUGCUCCUUACGAGGAG | 868 |
| CasPhi12 | ACUGACCUUCCCUGAAACUUCU | |
| R3003_ | CUUUCAAGACUAAUAGAUUGCUCCUUACGAGGAG | 869 |
| CasPhi12 | ACCAGGGAAGGUCAGAAGAGCU | |
| R3004_ | CUUUCAAGACUAAUAGAUUGCUCCUUACGAGGAG | 870 |
| CasPhi12 | ACAGGGAAGGUCAGAAGAGCUC | |
| R3005_ | CUUUCAAGACUAAUAGAUUGCUCCUUACGAGGAG | 871 |
| CasPhi12 | ACCUGCCCUGCCCACCACAGCC | |
| R3006_ | CUUUCAAGACUAAUAGAUUGCUCCUUACGAGGAG | 872 |
| CasPhi12 | ACCCUGCCCUGCCCACCACAGC | |
| R3007_ | CUUUCAAGACUAAUAGAUUGCUCCUUACGAGGAG | 873 |
| CasPhi12 | ACACACAUGCCCAGGCAGCACC | |
| R3008_ | CUUUCAAGACUAAUAGAUUGCUCCUUACGAGGAG | 874 |
| CasPhi12 | ACCACAUGCCCAGGCAGCACCU | |
| R3009_ | CUUUCAAGACUAAUAGAUUGCUCCUUACGAGGAG | 875 |
| CasPhi12 | ACCCUGCCCCACAAAGGGCCUG | |
| R3010_ | CUUUCAAGACUAAUAGAUUGCUCCUUACGAGGAG | 876 |
| CasPhi12 | ACGUGGGGCAGGGAAGCUGAGG | |
| R3011_ | CUUUCAAGACUAAUAGAUUGCUCCUUACGAGGAG | 877 |
| CasPhi12 | ACUGGGGCAGGGAAGCUGAGGC | |
| R3012_ | CUUUCAAGACUAAUAGAUUGCUCCUUACGAGGAG | 878 |
| CasPhi12 | ACCUGCCUCAGCUUCCCUGCCC | |
| R3013_ | CUUUCAAGACUAAUAGAUUGCUCCUUACGAGGAG | 879 |
| CasPhi12 | ACCAGGCCCAGCCAGCACUCUG | |
| R3014_ | CUUUCAAGACUAAUAGAUUGCUCCUUACGAGGAG | 880 |
| CasPhi12 | ACAGGCCCAGCCAGCACUCUGG | |
| R3015_ | CUUUCAAGACUAAUAGAUUGCUCCUUACGAGGAG | 881 |
| CasPhi12 | ACCACCCCAGCCCCUCACACCA | |
| R3016_ | CUUUCAAGACUAAUAGAUUGCUCCUUACGAGGAG | 882 |
| CasPhi12 | ACGGACCGUAGGAUGUCCCUCU | |

TABLE N
CasΦ.32 gRNAs targeting human PD1 in T cells

| | | SEQ |
|---|---|---|
| | Repeat + spacer RNA Sequence | ID |
| Name | (5′ → 3′) | NO |
| R2921_ | GCUGGGGACCGAUCCUGAUUGCUCGCUGCGGCGA | 883 |
| CasPhi32 | GACCCUUCCGCUCACCUCCGCCU | |
| R2922_ | GCUGGGGACCGAUCCUGAUUGCUCGCUGCGGCGA | 884 |
| CasPhi32 | GACCCUUCCGCUCACCUCCGCCU | |
| R2923_ | GCUGGGGACCGAUCCUGAUUGCUCGCUGCGGCGA | 885 |
| CasPhi32 | GACCGCUCACCUCCGCCUGAGCA | |
| R2924_ | GCUGGGGACCGAUCCUGAUUGCUCGCUGCGGCGA | 886 |
| CasPhi32 | GACUCCACUGCUCAGGCGGAGGU | |
| R2925_ | GCUGGGGACCGAUCCUGAUUGCUCGCUGCGGCGA | 887 |
| CasPhi32 | GACUAGCACCGCCCAGACGACUG | |
| R2926_ | GCUGGGGACCGAUCCUGAUUGCUCGCUGCGGCGA | 888 |
| CasPhi32 | GACAGGCAUGCAGAUCCCACAGG | |
| R2927_ | GCUGGGGACCGAUCCUGAUUGCUCGCUGCGGCGA | 889 |
| CasPhi32 | GACCACAGGCGCCCUGGCCAGUC | |
| R2928_ | GCUGGGGACCGAUCCUGAUUGCUCGCUGCGGCGA | 890 |
| CasPhi32 | GACUCUGGGCGGUGCUACAACUG | |
| R2929_ | GCUGGGGACCGAUCCUGAUUGCUCGCUGCGGCGA | 891 |
| CasPhi32 | GACGCAUGCCUGGAGCAGCCCCA | |
| R2930_ | GCUGGGGACCGAUCCUGAUUGCUCGCUGCGGCGA | 892 |
| CasPhi32 | GACUAGCACCGCCCAGACGACUG | |
| R2931_ | GCUGGGGACCGAUCCUGAUUGCUCGCUGCGGCGA | 893 |
| CasPhi32 | GACUGGCCGCCAGCCCAGUUGUA | |
| R2932_ | GCUGGGGACCGAUCCUGAUUGCUCGCUGCGGCGA | 894 |
| CasPhi32 | GACCUUCCGCUCACCUCCGCCUG | |
| R2933_ | GCUGGGGACCGAUCCUGAUUGCUCGCUGCGGCGA | 895 |
| CasPhi32 | GACCAGGGCCUGUCUGGGGAGUC | |
| R2934_ | GCUGGGGACCGAUCCUGAUUGCUCGCUGCGGCGA | 896 |
| CasPhi32 | GACUCCCCAGCCCUGCUCGUGGU | |
| R2935_ | GCUGGGGACCGAUCCUGAUUGCUCGCUGCGGCGA | 897 |
| CasPhi32 | GACGGUCACCACGAGCAGGGCUG | |
| R2936_ | GCUGGGGACCGAUCCUGAUUGCUCGCUGCGGCGA | 898 |
| CasPhi32 | GACUCCCCUUCGGUCACCACGAG | |
| R2937_ | GCUGGGGACCGAUCCUGAUUGCUCGCUGCGGCGA | 899 |
| CasPhi32 | GACGAGAAGCUGCAGGUGAAGGU | |
| R2938_ | GCUGGGGACCGAUCCUGAUUGCUCGCUGCGGCGA | 900 |
| CasPhi32 | GACACCUGCAGCUUCUCCAACAC | |
| R2939_ | GCUGGGGACCGAUCCUGAUUGCUCGCUGCGGCGA | 901 |
| CasPhi32 | GACUCCAACACAUCGGAGAGCUU | |
| R2940_ | GCUGGGGACCGAUCCUGAUUGCUCGCUGCGGCGA | 902 |
| CasPhi32 | GACGCACGAAGCUCUCCGAUGUG | |
| R2941_ | GCUGGGGACCGAUCCUGAUUGCUCGCUGCGGCGA | 903 |
| CasPhi32 | GACAGCACGAAGCUCUCCGAUGU | |
| R2942_ | GCUGGGGACCGAUCCUGAUUGCUCGCUGCGGCGA | 904 |
| CasPhi32 | GACGUGCUAAACUGGUACCGCAU | |
| R2943_ | GCUGGGGACCGAUCCUGAUUGCUCGCUGCGGCGA | 905 |
| CasPhi32 | GACCUGGGGCUCAUGCGGUACCA | |
| R2944_ | GCUGGGGACCGAUCCUGAUUGCUCGCUGCGGCGA | 906 |
| CasPhi32 | GACUCCGUCUGGUUGCUGGGGCU | |
| R2945_ | GCUGGGGACCGAUCCUGAUUGCUCGCUGCGGCGA | 907 |
| CasPhi32 | GACCCCGAGGACCGCAGCCAGCC | |
| R2946_ | GCUGGGGACCGAUCCUGAUUGCUCGCUGCGGCGA | 908 |
| CasPhi32 | GACUGUGACACGGAAGCGGCAGU | |
| R2947_ | GCUGGGGACCGAUCCUGAUUGCUCGCUGCGGCGA | 909 |
| CasPhi32 | GACCGUGUCACACAACUGCCCAA | |
| R2948_ | GCUGGGGACCGAUCCUGAUUGCUCGCUGCGGCGA | 910 |
| CasPhi32 | GACGGCAGUUGUGUGACACGGAA | |
| R2949_ | GCUGGGGACCGAUCCUGAUUGCUCGCUGCGGCGA | 911 |
| CasPhi32 | GACCACAUGAGCGUGGUCAGGGC | |
| R2950_ | GCUGGGGACCGAUCCUGAUUGCUCGCUGCGGCGA | 912 |
| CasPhi32 | GACCGCCGGGCCCUGACCACGCU | |
| R2951_ | GCUGGGGACCGAUCCUGAUUGCUCGCUGCGGCGA | 913 |
| CasPhi32 | GACGGGGCCAGGGAGAUGGCCCC | |
| R2952_ | GCUGGGGACCGAUCCUGAUUGCUCGCUGCGGCGA | 914 |
| CasPhi32 | GACAUCUGCGCCUUGGGGGCCAG | |
| R2953_ | GCUGGGGACCGAUCCUGAUUGCUCGCUGCGGCGA | 915 |
| CasPhi32 | GACGAUCUGCGCCUUGGGGGCCA | |
| R2954_ | GCUGGGGACCGAUCCUGAUUGCUCGCUGCGGCGA | 916 |
| CasPhi32 | GACCCAGACAGGCCCUGGAACCC | |
| R2955_ | GCUGGGGACCGAUCCUGAUUGCUCGCUGCGGCGA | 917 |
| CasPhi32 | GACCCAGCCCUGCUCGUGGUGAC | |
| R2956_ | GCUGGGGACCGAUCCUGAUUGCUCGCUGCGGCGA | 918 |
| CasPhi32 | GACUCUCUGGAAGGGCACAAAGG | |
| R2957_ | GCUGGGGACCGAUCCUGAUUGCUCGCUGCGGCGA | 919 |
| CasPhi32 | GACGUGCCCUUCCAGAGAGAAGG | |
| R2958_ | GCUGGGGACCGAUCCUGAUUGCUCGCUGCGGCGA | 920 |
| CasPhi32 | GACUGCCCUUCCAGAGAGAAGGG | |
| R2959_ | GCUGGGGACCGAUCCUGAUUGCUCGCUGCGGCGA | 921 |
| CasPhi32 | GACUGCCCUUCUCUCUGGAAGGG | |
| R2960_ | GCUGGGGACCGAUCCUGAUUGCUCGCUGCGGCGA | 922 |
| CasPhi32 | GACCAGAGAGAAGGGCAGAAGUG | |
| R2961_ | GCUGGGGACCGAUCCUGAUUGCUCGCUGCGGCGA | 923 |
| CasPhi32 | GACGAACUGGCCGGCUGGCCUGG | |
| R2962_ | GCUGGGGACCGAUCCUGAUUGCUCGCUGCGGCGA | 924 |
| CasPhi32 | GACGGAACUGGCCGGCUGGCCUG | |
| R2963_ | GCUGGGGACCGAUCCUGAUUGCUCGCUGCGGCGA | 925 |
| CasPhi32 | GACCAAACCCUGGUGGUUGGUGU | |
| R2964_ | GCUGGGGACCGAUCCUGAUUGCUCGCUGCGGCGA | 926 |
| CasPhi32 | GACGUGUCGUGGGCGGCCUGCUG | |
| R2965_ | GCUGGGGACCGAUCCUGAUUGCUCGCUGCGGCGA | 927 |
| CasPhi32 | GACCCUCGUGCGGCCCGGGAGCA | |
| R2966_ | GCUGGGGACCGAUCCUGAUUGCUCGCUGCGGCGA | 928 |
| CasPhi32 | GACUCCCUGCAGAGAAACACACU | |
| R2967_ | GCUGGGGACCGAUCCUGAUUGCUCGCUGCGGCGA | 929 |
| CasPhi32 | GACCUCUGCAGGGACAAUAGGAG | |
| R2968_ | GCUGGGGACCGAUCCUGAUUGCUCGCUGCGGCGA | 930 |
| CasPhi32 | GACUCUGCAGGGACAAUAGGAGC | |
| R2969_ | GCUGGGGACCGAUCCUGAUUGCUCGCUGCGGCGA | 931 |
| CasPhi32 | GACCUCCUCAAAGAAGGAGGACC | |
| R2970_ | GCUGGGGACCGAUCCUGAUUGCUCGCUGCGGCGA | 932 |
| CasPhi32 | GACUCCUCAAAGAAGGAGGACCC | |
| R2971_ | GCUGGGGACCGAUCCUGAUUGCUCGCUGCGGCGA | 933 |
| CasPhi32 | GACUCUGUGGACUAUGGGGAGCU | |
| R2972_ | GCUGGGGACCGAUCCUGAUUGCUCGCUGCGGCGA | 934 |
| CasPhi32 | GACUCUCGCCACUGGAAAUCCAG | |
| R2973_ | GCUGGGGACCGAUCCUGAUUGCUCGCUGCGGCGA | 935 |
| CasPhi32 | GACCCAGUGGCGAGAGAAGACCC | |
| R2974_ | GCUGGGGACCGAUCCUGAUUGCUCGCUGCGGCGA | 936 |
| CasPhi32 | GACCAGUGGCGAGAGAAGACCCC | |
| R2975_ | GCUGGGGACCGAUCCUGAUUGCUCGCUGCGGCGA | 937 |
| CasPhi32 | GACCGCUAGGAAAGACAAUGGUG | |
| R2976_ | GCUGGGGACCGAUCCUGAUUGCUCGCUGCGGCGA | 938 |
| CasPhi32 | GACUCUUUCCUAGCGGAAUGGGC | |
| R2977_ | GCUGGGGACCGAUCCUGAUUGCUCGCUGCGGCGA | 939 |
| CasPhi32 | GACCCUAGCGGAAUGGGCACCUC | |
| R2978_ | GCUGGGGACCGAUCCUGAUUGCUCGCUGCGGCGA | 940 |
| CasPhi32 | GACCUAGCGGAAUGGGCACCUCA | |
| R2979_ | GCUGGGGACCGAUCCUGAUUGCUCGCUGCGGCGA | 941 |
| CasPhi32 | GACGCCCCUCUGACCGGCUUCCU | |
| R2980_ | GCUGGGGACCGAUCCUGAUUGCUCGCUGCGGCGA | 942 |
| CasPhi32 | GACCUUGGCCACCAGUGUUCUGC | |
| R2981_ | GCUGGGGACCGAUCCUGAUUGCUCGCUGCGGCGA | 943 |
| CasPhi32 | GACGCCACCAGUGUUCUGCAGAC | |
| R2982_ | GCUGGGGACCGAUCCUGAUUGCUCGCUGCGGCGA | 944 |
| CasPhi32 | GACUGCAGACCCUCCACCAUGAG | |
| R2983_ | GCUGGGGACCGAUCCUGAUUGCUCGCUGCGGCGA | 945 |
| CasPhi32 | GACUCCUGAGGAAAUGCGCUGAC | |
| R2984_ | GCUGGGGACCGAUCCUGAUUGCUCGCUGCGGCGA | 946 |
| CasPhi32 | GACCCUCAGGAGAAGCAGGCAGG | |
| R2985_ | GCUGGGGACCGAUCCUGAUUGCUCGCUGCGGCGA | 947 |
| CasPhi32 | GACCUCAGGAGAAGCAGGCAGGG | |
| R2986_ | GCUGGGGACCGAUCCUGAUUGCUCGCUGCGGCGA | 948 |
| CasPhi32 | GACCAGGCCGUCCAGGGGCUGAG | |
| R2987_ | GCUGGGGACCGAUCCUGAUUGCUCGCUGCGGCGA | 949 |
| CasPhi32 | GACAGACAUGAGUCCUGUGGUGG | |
| R2988_ | GCUGGGGACCGAUCCUGAUUGCUCGCUGCGGCGA | 950 |
| CasPhi32 | GACAGGUCCUGCCAGCACAGAGC | |
| R2989_ | GCUGGGGACCGAUCCUGAUUGCUCGCUGCGGCGA | 951 |
| CasPhi32 | GACAGGGAGCUGGACGCAGGCAG | |
| R2990_ | GCUGGGGACCGAUCCUGAUUGCUCGCUGCGGCGA | 952 |
| CasPhi32 | GACAGCCCCGGGCCGCAGGCAGC | |
| R2991_ | GCUGGGGACCGAUCCUGAUUGCUCGCUGCGGCGA | 953 |
| CasPhi32 | GACAGGCAGGAGGCUCCGGGGCG | |
| R2992_ | GCUGGGGACCGAUCCUGAUUGCUCGCUGCGGCGA | 954 |
| CasPhi32 | GACGGGGCUGGUUGGAGAUGGCC | |
| R2993_ | GCUGGGGACCGAUCCUGAUUGCUCGCUGCGGCGA | 955 |
| CasPhi32 | GACGAGAUGGCCUUGGAGCAGCC | |
| R2994_ | GCUGGGGACCGAUCCUGAUUGCUCGCUGCGGCGA | 956 |
| CasPhi32 | GACGCUGCUCCAAGGCCAUCUCC | |
| R2995_ | GCUGGGGACCGAUCCUGAUUGCUCGCUGCGGCGA | 957 |
| CasPhi32 | GACGAGCAGCCAAGGUGCCCCUG | |
| R2996_ | GCUGGGGACCGAUCCUGAUUGCUCGCUGCGGCGA | 958 |
| CasPhi32 | GACGGGAUGCCACUGCCAGGGGC | |
| R2997_ | GCUGGGGACCGAUCCUGAUUGCUCGCUGCGGCGA | 959 |
| CasPhi32 | GACCGGGAUGCCACUGCCAGGGG | |
| R2998_ | GCUGGGGACCGAUCCUGAUUGCUCGCUGCGGCGA | 960 |
| CasPhi32 | GACGGCCCUGCGUCCAGGGCGUU | |
| R2999_ | GCUGGGGACCGAUCCUGAUUGCUCGCUGCGGCGA | 961 |
| CasPhi32 | GACUCUGCUCCCUGCAGGCCUAG | |
| R3000_ | GCUGGGGACCGAUCCUGAUUGCUCGCUGCGGCGA | 962 |
| CasPhi32 | GACUCUAGGCCUGCAGGGAGCAG | |
| R3001_ | GCUGGGGACCGAUCCUGAUUGCUCGCUGCGGCGA | 963 |
| CasPhi32 | GACCCUGAAACUUCUCUAGGCCU | |
| R3002_ | GCUGGGGACCGAUCCUGAUUGCUCGCUGCGGCGA | 964 |
| CasPhi32 | GACUGACCUUCCCUGAAACUUCU | |
| R3003_ | GCUGGGGACCGAUCCUGAUUGCUCGCUGCGGCGA | 965 |
| CasPhi32 | GACCAGGGAAGGUCAGAAGAGCU | |
| R3004_ | GCUGGGGACCGAUCCUGAUUGCUCGCUGCGGCGA | 966 |
| CasPhi32 | GACAGGGAAGGUCAGAAGAGCUC | |
| R3005_ | GCUGGGGACCGAUCCUGAUUGCUCGCUGCGGCGA | 967 |
| CasPhi32 | GACCUGCCCUGCCCACCACAGCC | |
| R3006_ | GCUGGGGACCGAUCCUGAUUGCUCGCUGCGGCGA | 968 |
| CasPhi32 | GACCCUGCCCUGCCCACCACAGC | |
| R3007_ | GCUGGGGACCGAUCCUGAUUGCUCGCUGCGGCGA | 969 |
| CasPhi32 | GACACACAUGCCCAGGCAGCACC | |
| R3008_ | GCUGGGGACCGAUCCUGAUUGCUCGCUGCGGCGA | 970 |
| CasPhi32 | GACCACAUGCCCAGGCAGCACCU | |
| R3009_ | GCUGGGGACCGAUCCUGAUUGCUCGCUGCGGCGA | 971 |
| CasPhi32 | GACCCUGCCCCACAAAGGGCCUG | |
| R3010_ | GCUGGGGACCGAUCCUGAUUGCUCGCUGCGGCGA | 972 |
| CasPhi32 | GACGUGGGGCAGGGAAGCUGAGG | |
| R3011_ | GCUGGGGACCGAUCCUGAUUGCUCGCUGCGGCGA | 973 |
| CasPhi32 | GACUGGGGCAGGGAAGCUGAGGC | |
| R3012_ | GCUGGGGACCGAUCCUGAUUGCUCGCUGCGGCGA | 974 |
| CasPhi32 | GACCUGCCUCAGCUUCCCUGCCC | |
| R3013_ | GCUGGGGACCGAUCCUGAUUGCUCGCUGCGGCGA | 975 |
| CasPhi32 | GACCAGGCCCAGCCAGCACUCUG | |
| R3014_ | GCUGGGGACCGAUCCUGAUUGCUCGCUGCGGCGA | 976 |
| CasPhi32 | GACAGGCCCAGCCAGCACUCUGG | |
| R3015_ | GCUGGGGACCGAUCCUGAUUGCUCGCUGCGGCGA | 977 |
| CasPhi32 | GACCACCCCAGCCCCUCACACCA | |
| R3016_ | GCUGGGGACCGAUCCUGAUUGCUCGCUGCGGCGA | 978 |
| CasPhi32 | GACGGACCGUAGGAUGUCCCUCU | |

TABLE O
CasΦ.12 gRNAs targeting human CIITA

| | | SEQ |
|---|---|---|
| | Repeat + spacer sequence | ID |
| Name | RNA Sequence (5′ → 3′) | NO |
| R4503_CasPhi12_ | CUUUCAAGACUAAUAGAUUGCUCCUUAC | 979 |
| C2TA_T1.1 | GAGGAGACCUACACAAUGCGUUGCCUGG | |
| R4504_CasPhi12_ | CUUUCAAGACUAAUAGAUUGCUCCUUAC | 980 |
| C2TA_T1.2 | GAGGAGACGGGCUCUGACAGGUAGGACC | |
| R4505_CasPhi12_ | CUUUCAAGACUAAUAGAUUGCUCCUUAC | 981 |
| C2TA_T1.3 | GAGGAGACUGUAGGAAUCCCAGCCAGGC | |
| R4506_CasPhi12_ | CUUUCAAGACUAAUAGAUUGCUCCUUAC | 982 |
| C2TA_T1.8 | GAGGAGACCCUGGCUCCACGCCCUGCUG | |
| R4507_CasPhi12_ | CUUUCAAGACUAAUAGAUUGCUCCUUAC | 983 |
| C2TA_T1.9 | GAGGAGACGGGAAGCUGAGGGCACGAGG | |
| R4508_CasPhi12_ | CUUUCAAGACUAAUAGAUUGCUCCUUAC | 984 |
| C2TA_T2.1 | GAGGAGACACAGCGAUGCUGACCCCCUG | |
| R4509_CasPhi12_ | CUUUCAAGACUAAUAGAUUGCUCCUUAC | 985 |
| C2TA_T2.2 | GAGGAGACUUAACAGCGAUGCUGACCCC | |
| R4510_CasPhi12_ | CUUUCAAGACUAAUAGAUUGCUCCUUAC | 986 |
| C2TA_T2.3 | GAGGAGACUAUGACCAGAUGGACCUGGC | |
| R4511_CasPhi12_ | CUUUCAAGACUAAUAGAUUGCUCCUUAC | 987 |
| C2TA_T2.4 | GAGGAGACGGGCCCCUAGAAGGUGGCUA | |
| R4512_CasPhi12_ | CUUUCAAGACUAAUAGAUUGCUCCUUAC | 988 |
| C2TA_T2.5 | GAGGAGACUAGGGGCCCCAACUCCAUGG | |
| R4513_CasPhi12_ | CUUUCAAGACUAAUAGAUUGCUCCUUAC | 989 |
| C2TA_T2.6 | GAGGAGACAGAAGCUCCAGGUAGCCACC | |
| R4514_CasPhi12_ | CUUUCAAGACUAAUAGAUUGCUCCUUAC | 990 |
| C2TA_T2.7 | GAGGAGACUCCAGCCAGGUCCAUCUGGU | |
| R4515_CasPhi12_ | CUUUCAAGACUAAUAGAUUGCUCCUUAC | 991 |
| C2TA_T2.8 | GAGGAGACUUCUCCAGCCAGGUCCAUCU | |
| R5200_CasPhi12 | CUUUCAAGACUAAUAGAUUGCUCCUUAC | 2112 |
| | GAGGAGACAGCAGGCUGUUGUGUGACAU | |
| R5201_CasPhi12 | CUUUCAAGACUAAUAGAUUGCUCCUUAC | 2113 |
| | GAGGAGACCAUGUCACACAACAGCCUGC | |
| R5202_CasPhi12 | CUUUCAAGACUAAUAGAUUGCUCCUUAC | 2114 |
| | GAGGAGACUGUGACAUGGAAGGUGAUGA | |
| R5203_CasPhi12 | CUUUCAAGACUAAUAGAUUGCUCCUUAC | 2115 |
| | GAGGAGACAUCACCUUCCAUGUCACACA | |
| R5204_CasPhi12 | CUUUCAAGACUAAUAGAUUGCUCCUUAC | 2116 |
| | GAGGAGACGCAUAAGCCUCCCUGGUCUC | |
| R5205_CasPhi12 | CUUUCAAGACUAAUAGAUUGCUCCUUAC | 2117 |
| | GAGGAGACCAGGACUCCCAGCUGGAGGG | |
| R5206_CasPhi12 | CUUUCAAGACUAAUAGAUUGCUCCUUAC | 2118 |
| | GAGGAGACCUCAGGCCCUCCAGCUGGGA | |
| R5207_CasPhi12 | CUUUCAAGACUAAUAGAUUGCUCCUUAC | 2119 |
| | GAGGAGACUGCUGGCAUCUCCAUACUCU | |
| R5208_CasPhi12 | CUUUCAAGACUAAUAGAUUGCUCCUUAC | 2120 |
| | GAGGAGACUGCCCAACUUCUGCUGGCAU | |
| R5209_CasPhi12 | CUUUCAAGACUAAUAGAUUGCUCCUUAC | 2121 |
| | GAGGAGACCUGCCCAACUUCUGCUGGCA | |
| R5210_CasPhi12 | CUUUCAAGACUAAUAGAUUGCUCCUUAC | 2122 |
| | GAGGAGACUCUGCCCAACUUCUGCUGGC | |
| R5211_CasPhi12 | CUUUCAAGACUAAUAGAUUGCUCCUUAC | 2123 |
| | GAGGAGACUGACUUUUCUGCCCAACUUC | |
| R5212_CasPhi12 | CUUUCAAGACUAAUAGAUUGCUCCUUAC | 2124 |
| | GAGGAGACCUGACUUUUCUGCCCAACUU | |
| R5213_CasPhi12 | CUUUCAAGACUAAUAGAUUGCUCCUUAC | 2125 |
| | GAGGAGACUCUGACUUUUCUGCCCAACU | |
| R5214_CasPhi12 | CUUUCAAGACUAAUAGAUUGCUCCUUAC | 2126 |
| | GAGGAGACCCAGAGGAGCUUCCGGCAGA | |
| R5215_CasPhi12 | CUUUCAAGACUAAUAGAUUGCUCCUUAC | 2127 |
| | GAGGAGACAGGUCUGCCGGAAGCUCCUC | |
| R5216_CasPhi12 | CUUUCAAGACUAAUAGAUUGCUCCUUAC | 2128 |
| | GAGGAGACCGGCAGACCUGAAGCACUGG | |
| R5217_CasPhi12 | CUUUCAAGACUAAUAGAUUGCUCCUUAC | 2129 |
| | GAGGAGACCAGUGCUUCAGGUCUGCCGG | |
| R5218_CasPhi12 | CUUUCAAGACUAAUAGAUUGCUCCUUAC | 2130 |
| | GAGGAGACAACAGCGCAGGCAGUGGCAG | |
| R5219_CasPhi12 | CUUUCAAGACUAAUAGAUUGCUCCUUAC | 2131 |
| | GAGGAGACAACCAGGAGCCAGCCUCCGG | |
| R5220_CasPhi12 | CUUUCAAGACUAAUAGAUUGCUCCUUAC | 2132 |
| | GAGGAGACUCCAGGCGCAUCUGGCCGGA | |
| R5221_CasPhi12 | CUUUCAAGACUAAUAGAUUGCUCCUUAC | 2133 |
| | GAGGAGACCUCCAGGCGCAUCUGGCCGG | |
| R5222_CasPhi12 | CUUUCAAGACUAAUAGAUUGCUCCUUAC | 2134 |
| | GAGGAGACUCUCCAGGCGCAUCUGGCCG | |
| R5223_CasPhi12 | CUUUCAAGACUAAUAGAUUGCUCCUUAC | 2135 |
| | GAGGAGACCUCCAGUUCCUCGUUGAGCU | |
| R5224_CasPhi12 | CUUUCAAGACUAAUAGAUUGCUCCUUAC | 2136 |
| | GAGGAGACUCCAGUUCCUCGUUGAGCUG | |
| R5225_CasPhi12 | CUUUCAAGACUAAUAGAUUGCUCCUUAC | 2137 |
| | GAGGAGACAGGCAGCUCAACGAGGAACU | |
| R5226_CasPhi12 | CUUUCAAGACUAAUAGAUUGCUCCUUAC | 2138 |
| | GAGGAGACCUCGUUGAGCUGCCUGAAUC | |
| R5227_CasPhi12 | CUUUCAAGACUAAUAGAUUGCUCCUUAC | 2139 |
| | GAGGAGACAGCUGCCUGAAUCUCCCUGA | |
| R5228_CasPhi12 | CUUUCAAGACUAAUAGAUUGCUCCUUAC | 2140 |
| | GAGGAGACGUCCCCACCAUCUCCACUCU | |
| R5229_CasPhi12 | CUUUCAAGACUAAUAGAUUGCUCCUUAC | 2141 |
| | GAGGAGACUCCCCACCAUCUCCACUCUG | |
| R5230_CasPhi12 | CUUUCAAGACUAAUAGAUUGCUCCUUAC | 2142 |
| | GAGGAGACCCAGAGCCCAUGGGGCAGAG | |
| R5231_CasPhi12 | CUUUCAAGACUAAUAGAUUGCUCCUUAC | 2143 |
| | GAGGAGACGCCAGAGCCCAUGGGGCAGA | |
| R5232_CasPhi12 | CUUUCAAGACUAAUAGAUUGCUCCUUAC | 2144 |
| | GAGGAGACCAGCCUCAGAGAUUUGCCAG | |
| R5233_CasPhi12 | CUUUCAAGACUAAUAGAUUGCUCCUUAC | 2145 |
| | GAGGAGACGGAGGCCGUGGACAGUGAAU | |
| R5234_CasPhi12 | CUUUCAAGACUAAUAGAUUGCUCCUUAC | 2146 |
| | GAGGAGACACUGUCCACGGCCUCCCAAC | |
| R5235_CasPhi12 | CUUUCAAGACUAAUAGAUUGCUCCUUAC | 2147 |
| | GAGGAGACGCUCCAUCAGCCACUGACCU | |
| R5236_CasPhi12 | CUUUCAAGACUAAUAGAUUGCUCCUUAC | 2148 |
| | GAGGAGACAGGCAUGCUGGGCAGGUCAG | |
| R5237_CasPhi12 | CUUUCAAGACUAAUAGAUUGCUCCUUAC | 2149 |
| | GAGGAGACCUCGGGAGGUCAGGGCAGGU | |
| R5238_CasPhi12 | CUUUCAAGACUAAUAGAUUGCUCCUUAC | 2150 |
| | GAGGAGACGCUCGGGAGGUCAGGGCAGG | |
| R5239_CasPhi12 | CUUUCAAGACUAAUAGAUUGCUCCUUAC | 2151 |
| | GAGGAGACGAGACCUCUCCAGCUGCCGG | |
| R5240_CasPhi12 | CUUUCAAGACUAAUAGAUUGCUCCUUAC | 2152 |
| | GAGGAGACUUGGAGACCUCUCCAGCUGC | |
| R5241_CasPhi12 | CUUUCAAGACUAAUAGAUUGCUCCUUAC | 2153 |
| | GAGGAGACGAAGCUUGUUGGAGACCUCU | |
| R5242_CasPhi12 | CUUUCAAGACUAAUAGAUUGCUCCUUAC | 2154 |
| | GAGGAGACGGAAGCUUGUUGGAGACCUC | |
| R5243_CasPhi12 | CUUUCAAGACUAAUAGAUUGCUCCUUAC | 2155 |
| | GAGGAGACUGGAAGCUUGUUGGAGACCU | |
| R5244_CasPhi12 | CUUUCAAGACUAAUAGAUUGCUCCUUAC | 2156 |
| | GAGGAGACUACCGCUCACUGCAGGACAC | |
| R5245_CasPhi12 | CUUUCAAGACUAAUAGAUUGCUCCUUAC | 2157 |
| | GAGGAGACCUGCUGCUCCUCUCCAGCCU | |
| R5246_CasPhi12 | CUUUCAAGACUAAUAGAUUGCUCCUUAC | 2158 |
| | GAGGAGACCCGCUCCAGGCUCUUGCUGC | |
| R5247_CasPhi12 | CUUUCAAGACUAAUAGAUUGCUCCUUAC | 2159 |
| | GAGGAGACUGCCCAGUCCGGGGUGGCCA | |
| R5248_CasPhi12 | CUUUCAAGACUAAUAGAUUGCUCCUUAC | 2160 |
| | GAGGAGACGGCCAGCUGCCGUUCUGCCC | |
| R5249_CasPhi12 | CUUUCAAGACUAAUAGAUUGCUCCUUAC | 2161 |
| | GAGGAGACGCAGCCAACAGCACCUCAGC | |
| R5250_CasPhi12 | CUUUCAAGACUAAUAGAUUGCUCCUUAC | 2162 |
| | GAGGAGACGCUGCCAAGGAGCACCGGCG | |
| R5251_CasPhi12 | CUUUCAAGACUAAUAGAUUGCUCCUUAC | 2163 |
| | GAGGAGACCCCAGCACAGCAAUCACUCG | |
| R5252_CasPhi12 | CUUUCAAGACUAAUAGAUUGCUCCUUAC | 2164 |
| | GAGGAGACGCCCAGCACAGCAAUCACUC | |
| R5253_CasPhi12 | CUUUCAAGACUAAUAGAUUGCUCCUUAC | 2165 |
| | GAGGAGACCUGUGCUGGGCAAAGCUGGU | |
| R5254_CasPhi12 | CUUUCAAGACUAAUAGAUUGCUCCUUAC | 2166 |
| | GAGGAGACCCCUGACCAGCUUUGCCCAG | |
| R5255_CasPhi12 | CUUUCAAGACUAAUAGAUUGCUCCUUAC | 2167 |
| | GAGGAGACGGCUGGGGCAGUGAGCCGGG | |
| R5256_CasPhi12 | CUUUCAAGACUAAUAGAUUGCUCCUUAC | 2168 |
| | GAGGAGACUGGCCGGCUUCCCCAGUACG | |
| R5257_CasPhi12 | CUUUCAAGACUAAUAGAUUGCUCCUUAC | 2169 |
| | GAGGAGACCCCAGUACGACUUUGUCUUC | |
| R5258_CasPhi12 | CUUUCAAGACUAAUAGAUUGCUCCUUAC | 2170 |
| | GAGGAGACGUCUUCUCUGUCCCCUGCCA | |
| R5259_CasPhi12 | CUUUCAAGACUAAUAGAUUGCUCCUUAC | 2171 |
| | GAGGAGACUCUUCUCUGUCCCCUGCCAU | |
| R5260_CasPhi12 | CUUUCAAGACUAAUAGAUUGCUCCUUAC | 2172 |
| | GAGGAGACUCUGUCCCCUGCCAUUGCUU | |
| R5261_CasPhi12 | CUUUCAAGACUAAUAGAUUGCUCCUUAC | 2173 |
| | GAGGAGACAAGCAAUGGCAGGGGACAGA | |
| R5262_CasPhi12 | CUUUCAAGACUAAUAGAUUGCUCCUUAC | 2174 |
| | GAGGAGACCUUGAACCGUCCGGGGGAUG | |
| R5263_CasPhi12 | CUUUCAAGACUAAUAGAUUGCUCCUUAC | 2175 |
| | GAGGAGACAACCGUCCGGGGGAUGCCUA | |
| R5264_CasPhi12 | CUUUCAAGACUAAUAGAUUGCUCCUUAC | 2176 |
| | GAGGAGACUCCCUGGGCCCACAGCCACU | |
| R5265_CasPhi12 | CUUUCAAGACUAAUAGAUUGCUCCUUAC | 2177 |
| | GAGGAGACAAGAUGUGGCUGAAAACCUC | |
| R5266_CasPhi12 | CUUUCAAGACUAAUAGAUUGCUCCUUAC | 2178 |
| | GAGGAGACUCAGCCACAUCUUGAAGAGA | |
| R5267_CasPhi12 | CUUUCAAGACUAAUAGAUUGCUCCUUAC | 2179 |
| | GAGGAGACCAGCCACAUCUUGAAGAGAC | |
| R5268_CasPhi12 | CUUUCAAGACUAAUAGAUUGCUCCUUAC | 2180 |
| | GAGGAGACAGCCACAUCUUGAAGAGACC | |
| R5269_CasPhi12 | CUUUCAAGACUAAUAGAUUGCUCCUUAC | 2181 |
| | GAGGAGACAAGAGACCUGACCGCGUUCU | |
| R5270_CasPhi12 | CUUUCAAGACUAAUAGAUUGCUCCUUAC | 2182 |
| | GAGGAGACUGCUCAUCCUAGACGGCUUC | |
| R5271_CasPhi12 | CUUUCAAGACUAAUAGAUUGCUCCUUAC | 2183 |
| | GAGGAGACCAGCUCCUCGAAGCCGUCUA | |
| R5272_CasPhi12 | CUUUCAAGACUAAUAGAUUGCUCCUUAC | 2184 |
| | GAGGAGACCGCUUCCAGCUCCUCGAAGC | |
| R5273_CasPhi12 | CUUUCAAGACUAAUAGAUUGCUCCUUAC | 2185 |
| | GAGGAGACGAGGAGCUGGAAGCGCAAGA | |
| R5274_CasPhi12 | CUUUCAAGACUAAUAGAUUGCUCCUUAC | 2186 |
| | GAGGAGACCUGCACAGCACGUGCGGACC | |
| R5275_CasPhi12 | CUUUCAAGACUAAUAGAUUGCUCCUUAC | 2187 |
| | GAGGAGACUGGAAAAGGCCGGCCAGCAG | |
| R5276_CasPhi12 | CUUUCAAGACUAAUAGAUUGCUCCUUAC | 2188 |
| | GAGGAGACUUCUGGAAAAGGCCGGCCAG | |
| R5277_CasPhi12 | CUUUCAAGACUAAUAGAUUGCUCCUUAC | 2189 |
| | GAGGAGACUCCAGAAGAAGCUGCUCCGA | |
| R5278_CasPhi12 | CUUUCAAGACUAAUAGAUUGCUCCUUAC | 2190 |
| | GAGGAGACCCAGAAGAAGCUGCUCCGAG | |
| R5279_CasPhi12 | CUUUCAAGACUAAUAGAUUGCUCCUUAC | 2191 |
| | GAGGAGACCAGAAGAAGCUGCUCCGAGG | |
| R5280_CasPhi12 | CUUUCAAGACUAAUAGAUUGCUCCUUAC | 2192 |
| | GAGGAGACCACCCUCCUCCUCACAGCCC | |
| R5281_CasPhi12 | CUUUCAAGACUAAUAGAUUGCUCCUUAC | 2193 |
| | GAGGAGACCUCAGGCUCUGGACCAGGCG | |
| R5282_CasPhi12 | CUUUCAAGACUAAUAGAUUGCUCCUUAC | 2194 |
| | GAGGAGACGAGCUGUCCGGCUUCUCCAU | |
| R5283_CasPhi12 | CUUUCAAGACUAAUAGAUUGCUCCUUAC | 2195 |
| | GAGGAGACAGCUGUCCGGCUUCUCCAUG | |
| R5284_CasPhi12 | CUUUCAAGACUAAUAGAUUGCUCCUUAC | 2196 |
| | GAGGAGACUCCAUGGAGCAGGCCCAGGC | |
| R5285_CasPhi12 | CUUUCAAGACUAAUAGAUUGCUCCUUAC | 2197 |
| | GAGGAGACGAGAGCUCAGGGAUGACAGA | |
| R5286_CasPhi12 | CUUUCAAGACUAAUAGAUUGCUCCUUAC | 2198 |
| | GAGGAGACAGAGCUCAGGGAUGACAGAG | |
| R5287_CasPhi12 | CUUUCAAGACUAAUAGAUUGCUCCUUAC | 2199 |
| | GAGGAGACGUGCUCUGUCAUCCCUGAGC | |
| R5288_CasPhi12 | CUUUCAAGACUAAUAGAUUGCUCCUUAC | 2200 |
| | GAGGAGACUUCUCAGUCACAGCCACAGC | |
| R5289_CasPhi12 | CUUUCAAGACUAAUAGAUUGCUCCUUAC | 2201 |
| | GAGGAGACUCAGUCACAGCCACAGCCCU | |
| R5290_CasPhi12 | CUUUCAAGACUAAUAGAUUGCUCCUUAC | 2202 |
| | GAGGAGACGUGCCGGGCAGUGUGCCAGC | |
| R5291_CasPhi12 | CUUUCAAGACUAAUAGAUUGCUCCUUAC | 2203 |
| | GAGGAGACUGCCGGGCAGUGUGCCAGCU | |
| R5292_CasPhi12 | CUUUCAAGACUAAUAGAUUGCUCCUUAC | 2204 |
| | GAGGAGACGCGUCCUCCCCAAGCUCCAG | |
| R5293_CasPhi12 | CUUUCAAGACUAAUAGAUUGCUCCUUAC | 2205 |
| | GAGGAGACGGGAGGACGCCAAGCUGCCC | |
| R5294_CasPhi12 | CUUUCAAGACUAAUAGAUUGCUCCUUAC | 2206 |
| | GAGGAGACGCCAGCUCUGCCAGGGCCCC | |
| R5295_CasPhi12 | CUUUCAAGACUAAUAGAUUGCUCCUUAC | 2207 |
| | GAGGAGACAUGUCUGCGGCCCAGCUCCC | |
| R5392_CasPhi12 | CUUUCAAGACUAAUAGAUUGCUCCUUAC | 2208 |
| | GAGGAGACGAUGUCUGCGGCCCAGCUCC | |
| R5393_CasPhi12 | CUUUCAAGACUAAUAGAUUGCUCCUUAC | 2209 |
| | GAGGAGACCCAUCCGCAGACGUGAGGAC | |
| R5394_CasPhi12 | CUUUCAAGACUAAUAGAUUGCUCCUUAC | 2210 |
| | GAGGAGACGCCAUCGCCCAGGUCCUCAC | |
| R5395_CasPhi12 | CUUUCAAGACUAAUAGAUUGCUCCUUAC | 2211 |
| | GAGGAGACGGCCAUCGCCCAGGUCCUCA | |
| R5396_CasPhi12 | CUUUCAAGACUAAUAGAUUGCUCCUUAC | 2212 |
| | GAGGAGACGACUAAGCCUUUGGCCAUCG | |
| R5397_CasPhi12 | CUUUCAAGACUAAUAGAUUGCUCCUUAC | 2213 |
| | GAGGAGACGUCCAACACCCACCGCGGGC | |
| R5398_CasPhi12 | CUUUCAAGACUAAUAGAUUGCUCCUUAC | 2214 |
| | GAGGAGACCAGGAGGAAGCUGGGGAAGG | |
| R5399_CasPhi12 | CUUUCAAGACUAAUAGAUUGCUCCUUAC | 2215 |
| | GAGGAGACCCCAGCUUCCUCCUGCAAUG | |
| R5400_CasPhi12 | CUUUCAAGACUAAUAGAUUGCUCCUUAC | 2216 |
| | GAGGAGACCUCCUGCAAUGCUUCCUGGG | |
| R5401_CasPhi12 | CUUUCAAGACUAAUAGAUUGCUCCUUAC | 2217 |
| | GAGGAGACCUGGGGGCCCUGUGGCUGGC | |
| R5402_CasPhi12 | CUUUCAAGACUAAUAGAUUGCUCCUUAC | 2218 |
| | GAGGAGACGCCACUCAGAGCCAGCCACA | |
| R5403_CasPhi12 | CUUUCAAGACUAAUAGAUUGCUCCUUAC | 2219 |
| | GAGGAGACCGCCACUCAGAGCCAGCCAC | |
| R5404_CasPhi12 | CUUUCAAGACUAAUAGAUUGCUCCUUAC | 2220 |
| | GAGGAGACAUUUCGCCACUCAGAGCCAG | |
| R5405_CasPhi12 | CUUUCAAGACUAAUAGAUUGCUCCUUAC | 2221 |
| | GAGGAGACUCCUUGAUUUCGCCACUCAG | |
| R5406_CasPhi12 | CUUUCAAGACUAAUAGAUUGCUCCUUAC | 2222 |
| | GAGGAGACGGGUCAAUGCUAGGUACUGC | |
| R5407_CasPhi12 | CUUUCAAGACUAAUAGAUUGCUCCUUAC | 2223 |
| | GAGGAGACCUUGGGGUCAAUGCUAGGUA | |
| R5408_CasPhi12 | CUUUCAAGACUAAUAGAUUGCUCCUUAC | 2224 |
| | GAGGAGACUUCCUUGGGGUCAAUGCUAG | |
| R5409_CasPhi12 | CUUUCAAGACUAAUAGAUUGCUCCUUAC | 2225 |
| | GAGGAGACACCCCAAGGAAGAAGAGGCC | |
| R5410_CasPhi12 | CUUUCAAGACUAAUAGAUUGCUCCUUAC | 2226 |
| | GAGGAGACUCAUAGGGCCUCUUCUUCCU | |
| R5411_CasPhi12 | CUUUCAAGACUAAUAGAUUGCUCCUUAC | 2227 |
| | GAGGAGACCUGGCUGGGCUGAUCUUCCA | |
| R5412_CasPhi12 | CUUUCAAGACUAAUAGAUUGCUCCUUAC | 2228 |
| | GAGGAGACUGGCUGGGCUGAUCUUCCAG | |
| R5413_CasPhi12 | CUUUCAAGACUAAUAGAUUGCUCCUUAC | 2229 |
| | GAGGAGACCAGCCUCCCGCCCGCUGCCU | |
| R5414_CasPhi12 | CUUUCAAGACUAAUAGAUUGCUCCUUAC | 2230 |
| | GAGGAGACCUGUCCACCGAGGCAGCCGC | |
| R5415_CasPhi12 | CUUUCAAGACUAAUAGAUUGCUCCUUAC | 2231 |
| | GAGGAGACUGCUUCCUGUCCACCGAGGC | |
| R5416_CasPhi12 | CUUUCAAGACUAAUAGAUUGCUCCUUAC | 2232 |
| | GAGGAGACAGGUACCUCGCAAGCACCUU | |
| R5417_CasPhi12 | CUUUCAAGACUAAUAGAUUGCUCCUUAC | 2233 |
| | GAGGAGACCGAGGUACCUGAAGCGGCUG | |
| R5418_CasPhi12 | CUUUCAAGACUAAUAGAUUGCUCCUUAC | 2234 |
| | GAGGAGACCAGCCUCCUCGGCCUCGUGG | |
| R5419_CasPhi12 | CUUUCAAGACUAAUAGAUUGCUCCUUAC | 2235 |
| | GAGGAGACGGCAGCACGUGGUACAGGAG | |
| R5420_CasPhi12 | CUUUCAAGACUAAUAGAUUGCUCCUUAC | 2236 |
| | GAGGAGACGCAGCACGUGGUACAGGAGC | |
| R5421_CasPhi12 | CUUUCAAGACUAAUAGAUUGCUCCUUAC | 2237 |
| | GAGGAGACUCUGGGCACCCGCCUCACGC | |
| R5422_CasPhi12 | CUUUCAAGACUAAUAGAUUGCUCCUUAC | 2238 |
| | GAGGAGACCUGGGCACCCGCCUCACGCC | |
| R5423_CasPhi12 | CUUUCAAGACUAAUAGAUUGCUCCUUAC | 2239 |
| | GAGGAGACUGGGCACCCGCCUCACGCCU | |
| R5424_CasPhi12 | CUUUCAAGACUAAUAGAUUGCUCCUUAC | 2240 |
| | GAGGAGACCCCAGUACAUGUGCAUCAGG | |
| R5425_CasPhi12 | CUUUCAAGACUAAUAGAUUGCUCCUUAC | 2241 |
| | GAGGAGACGCCCGCCGCCUCCAAGGCCU | |
| R5426_CasPhi12 | CUUUCAAGACUAAUAGAUUGCUCCUUAC | 2242 |
| | GAGGAGACGAGGCGGCGGGCCAAGACUU | |
| R5427_CasPhi12 | CUUUCAAGACUAAUAGAUUGCUCCUUAC | 2243 |
| | GAGGAGACUCCCUGGACCUCCGCAGCAC | |
| R5428_CasPhi12 | CUUUCAAGACUAAUAGAUUGCUCCUUAC | 2244 |
| | GAGGAGACGCCCCUCUGGAUUGGGGAGC | |
| R5429_CasPhi12 | CUUUCAAGACUAAUAGAUUGCUCCUUAC | 2245 |
| | GAGGAGACCCCCUCUGGAUUGGGGAGCC | |
| R5430_CasPhi12 | CUUUCAAGACUAAUAGAUUGCUCCUUAC | 2246 |
| | GAGGAGACGGGAGCCUCGUGGGACUCAG | |
| R5431_CasPhi12 | CUUUCAAGACUAAUAGAUUGCUCCUUAC | 2247 |
| | GAGGAGACGUCUCCCCAUGCUGCUGCAG | |
| R5432_CasPhi12 | CUUUCAAGACUAAUAGAUUGCUCCUUAC | 2248 |
| | GAGGAGACUCCUCUGCUGCCUGAAGUAG | |
| R5433_CasPhi12 | CUUUCAAGACUAAUAGAUUGCUCCUUAC | 2249 |
| | GAGGAGACAGGCAGCAGAGGAGAAGUUC | |
| R5434_CasPhi12 | CUUUCAAGACUAAUAGAUUGCUCCUUAC | 2250 |
| | GAGGAGACAAAGGCUCGAUGGUGAACUU | |
| R5435_CasPhi12 | CUUUCAAGACUAAUAGAUUGCUCCUUAC | 2251 |
| | GAGGAGACGAAAGGCUCGAUGGUGAACU | |
| R5436_CasPhi12 | CUUUCAAGACUAAUAGAUUGCUCCUUAC | 2252 |
| | GAGGAGACACCAUCGAGCCUUUCAAAGC | |
| R5437_CasPhi12 | CUUUCAAGACUAAUAGAUUGCUCCUUAC | 2253 |
| | GAGGAGACGCUUUGAAAGGCUCGAUGGU | |
| R5438_CasPhi12 | CUUUCAAGACUAAUAGAUUGCUCCUUAC | 2254 |
| | GAGGAGACAGGGACUUGGCUUUGAAAGG | |
| R5439_CasPhi12 | CUUUCAAGACUAAUAGAUUGCUCCUUAC | 2255 |
| | GAGGAGACCAAAGCCAAGUCCCUGAAGG | |
| R5440_CasPhi12 | CUUUCAAGACUAAUAGAUUGCUCCUUAC | 2256 |
| | GAGGAGACAAAGCCAAGUCCCUGAAGGA | |
| R5441_CasPhi12 | CUUUCAAGACUAAUAGAUUGCUCCUUAC | 2257 |
| | GAGGAGACCACAUCCUUCAGGGACUUGG | |
| R5442_CasPhi12 | CUUUCAAGACUAAUAGAUUGCUCCUUAC | 2258 |
| | GAGGAGACCCAGGUCUUCCACAUCCUUC | |
| R5443_CasPhi12 | CUUUCAAGACUAAUAGAUUGCUCCUUAC | 2259 |
| | GAGGAGACCCCAGGUCUUCCACAUCCUU | |
| R5444_CasPhi12 | CUUUCAAGACUAAUAGAUUGCUCCUUAC | 2260 |
| | GAGGAGACCUCGGAAGACACAGCUGGGG | |
| R5445_CasPhi12 | CUUUCAAGACUAAUAGAUUGCUCCUUAC | 2261 |
| | GAGGAGACGGUCCCGAACAGCAGGGAGC | |
| R5446_CasPhi12 | CUUUCAAGACUAAUAGAUUGCUCCUUAC | 2262 |
| | GAGGAGACAGGUCCCGAACAGCAGGGAG | |
| R5447_CasPhi12 | CUUUCAAGACUAAUAGAUUGCUCCUUAC | 2263 |
| | GAGGAGACUUUAGGUCCCGAACAGCAGG | |
| R5448_CasPhi12 | CUUUCAAGACUAAUAGAUUGCUCCUUAC | 2264 |
| | GAGGAGACCUUUAGGUCCCGAACAGCAG | |
| R5449_CasPhi12 | CUUUCAAGACUAAUAGAUUGCUCCUUAC | 2265 |
| | GAGGAGACGGGACCUAAAGAAACUGGAG | |
| R5450_CasPhi12 | CUUUCAAGACUAAUAGAUUGCUCCUUAC | 2266 |
| | GAGGAGACGGGAAAGCCUGGGGGCCUGA | |
| R5451_CasPhi12 | CUUUCAAGACUAAUAGAUUGCUCCUUAC | 2267 |
| | GAGGAGACGGGGAAAGCCUGGGGGCCUG | |
| R5452_CasPhi12 | CUUUCAAGACUAAUAGAUUGCUCCUUAC | 2268 |
| | GAGGAGACCCCCAAACUGGUGCGGAUCC | |
| R5453_CasPhi12 | CUUUCAAGACUAAUAGAUUGCUCCUUAC | 2269 |
| | GAGGAGACCCCAAACUGGUGCGGAUCCU | |
| R5454_CasPhi12 | CUUUCAAGACUAAUAGAUUGCUCCUUAC | 2270 |
| | GAGGAGACUUCUCACUCAGCGCAUCCAG | |
| R5455_CasPhi12 | CUUUCAAGACUAAUAGAUUGCUCCUUAC | 2271 |
| | GAGGAGACAGCUGGGGGAAGGUGGCUGA | |
| R5456_CasPhi12 | CUUUCAAGACUAAUAGAUUGCUCCUUAC | 2272 |
| | GAGGAGACCCCCAGCUGAAGUCCUUGGA | |
| R5457_CasPhi12 | CUUUCAAGACUAAUAGAUUGCUCCUUAC | 2273 |
| | GAGGAGACCAAGGACUUCAGCUGGGGGA | |
| R5458_CasPhi12 | CUUUCAAGACUAAUAGAUUGCUCCUUAC | 2274 |
| | GAGGAGACCCAAGGACUUCAGCUGGGGG | |
| R5459_CasPhi12 | CUUUCAAGACUAAUAGAUUGCUCCUUAC | 2275 |
| | GAGGAGACAGGGUUUCCAAGGACUUCAG | |
| R5460_CasPhi12 | CUUUCAAGACUAAUAGAUUGCUCCUUAC | 2276 |
| | GAGGAGACUAGGCACCCAGGUCAGUGAU | |
| R5461_CasPhi12 | CUUUCAAGACUAAUAGAUUGCUCCUUAC | 2277 |
| | GAGGAGACGUAGGCACCCAGGUCAGUGA | |
| R5462_CasPhi12 | CUUUCAAGACUAAUAGAUUGCUCCUUAC | 2278 |
| | GAGGAGACGCUCGCUGCAUCCCUGCUCA | |
| R5463_CasPhi12 | CUUUCAAGACUAAUAGAUUGCUCCUUAC | 2279 |
| | GAGGAGACGCCUGAGCAGGGAUGCAGCG | |
| R5464_CasPhi12 | CUUUCAAGACUAAUAGAUUGCUCCUUAC | 2280 |
| | GAGGAGACUACAAUAACUGCAUCUGCGA | |
| R5465_CasPhi12 | CUUUCAAGACUAAUAGAUUGCUCCUUAC | 2281 |
| | GAGGAGACGCUCGUGUGCUUCCGGACAU | |
| R5466_CasPhi12 | CUUUCAAGACUAAUAGAUUGCUCCUUAC | 2282 |
| | GAGGAGACCGGACAUGGUGUCCCUCCGG | |
| R5467_CasPhi12 | CUUUCAAGACUAAUAGAUUGCUCCUUAC | 2283 |
| | GAGGAGACACGGCUGCCGGGGCCCAGCA | |
| R5468_CasPhi12 | CUUUCAAGACUAAUAGAUUGCUCCUUAC | 2284 |
| | GAGGAGACGGAGGUGUCCUCAUGUGGAG | |
| R5469_CasPhi12 | CUUUCAAGACUAAUAGAUUGCUCCUUAC | 2285 |
| | GAGGAGACCUGGACACUGAAUGGGAUGG | |
| R5470_CasPhi12 | CUUUCAAGACUAAUAGAUUGCUCCUUAC | 2286 |
| | GAGGAGACAGUGUCCAGGAACACCUGCA | |
| R5471_CasPhi12 | CUUUCAAGACUAAUAGAUUGCUCCUUAC | 2287 |
| | GAGGAGACCAGGUGUUCCUGGACACUGA | |
| R5472_CasPhi12 | CUUUCAAGACUAAUAGAUUGCUCCUUAC | 2288 |
| | GAGGAGACUUGCAGGUGUUCCUGGACAC | |
| R5473_CasPhi12 | CUUUCAAGACUAAUAGAUUGCUCCUUAC | 2289 |
| | GAGGAGACACGGAUCAGCCUGAGAUGAU | |

TABLE P
CasΦ.32 gRNAs targeting human CIITA

| | | SEQ |
|---|---|---|
| | Repeat + spacer sequence | ID |
| Name | RNA Sequence (5′ → 3′) | NO |
| R4503_ | GCUGGGGACCGAUCCUGAUUGCUCGCUGCGGCG | 992 |
| CasPhi32_ | AGACCUACACAAUGCGUUGCCUGG | |
| C2TA_T1.1 | | |
| R4504_ | GCUGGGGACCGAUCCUGAUUGCUCGCUGCGGCG | 993 |
| CasPhi32_ | AGACGGGCUCUGACAGGUAGGACC | |
| C2TA_T1.2 | | |
| R4505_ | GCUGGGGACCGAUCCUGAUUGCUCGCUGCGGCG | 994 |
| CasPhi32_ | AGACUGUAGGAAUCCCAGCCAGGC | |
| C2TA_T1.3 | | |
| R4506_ | GCUGGGGACCGAUCCUGAUUGCUCGCUGCGGCG | 995 |
| CasPhi32_ | AGACCCUGGCUCCACGCCCUGCUG | |
| C2TA_T1.8 | | |
| R4507_ | GCUGGGGACCGAUCCUGAUUGCUCGCUGCGGCG | 996 |
| CasPhi32_ | AGACGGGAAGCUGAGGGCACGAGG | |
| C2TA_T1.9 | | |
| R4508_ | GCUGGGGACCGAUCCUGAUUGCUCGCUGCGGCG | 997 |
| CasPhi32_ | AGACACAGCGAUGCUGACCCCCUG | |
| C2TA_T2.1 | | |
| R4509_ | GCUGGGGACCGAUCCUGAUUGCUCGCUGCGGCG | 998 |
| CasPhi32_ | AGACUUAACAGCGAUGCUGACCCC | |
| C2TA_T2.2 | | |
| R4510_ | GCUGGGGACCGAUCCUGAUUGCUCGCUGCGGCG | 999 |
| CasPhi32_ | AGACUAUGACCAGAUGGACCUGGC | |
| C2TA_T2.3 | | |
| R4511_ | GCUGGGGACCGAUCCUGAUUGCUCGCUGCGGCG | 1000 |
| CasPhi32_ | AGACGGGCCCCUAGAAGGUGGCUA | |
| C2TA_T2.4 | | |
| R4512_ | GCUGGGGACCGAUCCUGAUUGCUCGCUGCGGCG | 1001 |
| CasPhi32_ | AGACUAGGGGCCCCAACUCCAUGG | |
| C2TA_T2.5 | | |
| R4513_ | GCUGGGGACCGAUCCUGAUUGCUCGCUGCGGCG | 1002 |
| CasPhi32_ | AGACAGAAGCUCCAGGUAGCCACC | |
| C2TA_T2.6 | | |
| R4514_ | GCUGGGGACCGAUCCUGAUUGCUCGCUGCGGCG | 1003 |
| CasPhi32_ | AGACUCCAGCCAGGUCCAUCUGGU | |
| C2TA_T2.7 | | |
| R4515_ | GCUGGGGACCGAUCCUGAUUGCUCGCUGCGGCG | 1004 |
| CasPhi32_ | AGACUUCUCCAGCCAGGUCCAUCU | |
| C2TA_T2.8 | | |

TABLE Q
CasΦ.12 gRNAs targeting mouse PCSK9

| | | SEQ |
|---|---|---|
| | Repeat + spacer sequence | ID |
| Name | RNA Sequence (5′ → 3′) | NO |
| R4238_ | CUUUCAAGACUAAUAGAUUGCUCCUUACGAGGAG | 1005 |
| CasPhi12 | ACCCGCUGUUGCCGCCGCUGCU | |
| R4239_ | CUUUCAAGACUAAUAGAUUGCUCCUUACGAGGAG | 1006 |
| CasPhi12 | ACCCGCCGCUGCUGCUGCUGUU | |
| R4240_ | CUUUCAAGACUAAUAGAUUGCUCCUUACGAGGAG | 1007 |
| CasPhi12 | ACCUGCUACUGUGCCCCACCGG | |
| R4241_ | CUUUCAAGACUAAUAGAUUGCUCCUUACGAGGAG | 1008 |
| CasPhi12 | ACAUAAUCUCCAUCCUCGUCCU | |
| R4242_ | CUUUCAAGACUAAUAGAUUGCUCCUUACGAGGAG | 1009 |
| CasPhi12 | ACUGAAGAGCUGAUGCUCGCCC | |
| R4243_ | CUUUCAAGACUAAUAGAUUGCUCCUUACGAGGAG | 1010 |
| CasPhi12 | ACGAGCAACGGCGGAAGGUGGC | |
| R4244_ | CUUUCAAGACUAAUAGAUUGCUCCUUACGAGGAG | 1011 |
| CasPhi12 | ACCUGGCAGCCUCCAGGCCUCC | |
| R4245_ | CUUUCAAGACUAAUAGAUUGCUCCUUACGAGGAG | 1012 |
| CasPhi12 | ACUGGUGCUGAUGGAGGAGACC | |
| R4246_ | CUUUCAAGACUAAUAGAUUGCUCCUUACGAGGAG | 1013 |
| CasPhi12 | ACAAUCUGUAGCCUCUGGGUCU | |
| R4247_ | CUUUCAAGACUAAUAGAUUGCUCCUUACGAGGAG | 1014 |
| CasPhi12 | ACUUCAAUCUGUAGCCUCUGGG | |
| R4248_ | CUUUCAAGACUAAUAGAUUGCUCCUUACGAGGAG | 1015 |
| CasPhi12 | ACGUUCAAUCUGUAGCCUCUGG | |
| R4249_ | CUUUCAAGACUAAUAGAUUGCUCCUUACGAGGAG | 1016 |
| CasPhi12 | ACAACAAACUGCCCACCGCCUG | |
| R4250_ | CUUUCAAGACUAAUAGAUUGCUCCUUACGAGGAG | 1017 |
| CasPhi12 | ACAUGACAUAGCCCCGGCGGGC | |
| R4251_ | CUUUCAAGACUAAUAGAUUGCUCCUUACGAGGAG | 1018 |
| CasPhi12 | ACUACAUAUCUUUUAUGACCUC | |
| R4252_ | CUUUCAAGACUAAUAGAUUGCUCCUUACGAGGAG | 1019 |
| CasPhi12 | ACUAUGACCUCUUCCCUGGCUU | |
| R4253_ | CUUUCAAGACUAAUAGAUUGCUCCUUACGAGGAG | 1020 |
| CasPhi12 | ACAUGACCUCUUCCCUGGCUUC | |
| R4254_ | CUUUCAAGACUAAUAGAUUGCUCCUUACGAGGAG | 1021 |
| CasPhi12 | ACUGACCUCUUCCCUGGCUUCU | |
| R4255_ | CUUUCAAGACUAAUAGAUUGCUCCUUACGAGGAG | 1022 |
| CasPhi12 | ACACCAAGAAGCCAGGGAAGAG | |
| R4256_ | CUUUCAAGACUAAUAGAUUGCUCCUUACGAGGAG | 1023 |
| CasPhi12 | ACCCUGGCUUCUUGGUGAAGAU | |
| R4257_ | CUUUCAAGACUAAUAGAUUGCUCCUUACGAGGAG | 1024 |
| CasPhi12 | ACUUGGUGAAGAUGAGCAGUGA | |
| R4258_ | CUUUCAAGACUAAUAGAUUGCUCCUUACGAGGAG | 1025 |
| CasPhi12 | ACGUGAAGAUGAGCAGUGACCU | |
| R4259_ | CUUUCAAGACUAAUAGAUUGCUCCUUACGAGGAG | 1026 |
| CasPhi12 | ACCCCCAUGUGGAGUACAUUGA | |
| R4260_ | CUUUCAAGACUAAUAGAUUGCUCCUUACGAGGAG | 1027 |
| CasPhi12 | ACCUCAAUGUACUCCACAUGGG | |
| R4261_ | CUUUCAAGACUAAUAGAUUGCUCCUUACGAGGAG | 1028 |
| CasPhi12 | ACAGGAAGACUCCUUUGUCUUC | |
| R4262_ | CUUUCAAGACUAAUAGAUUGCUCCUUACGAGGAG | 1029 |
| CasPhi12 | ACGUCUUCGCCCAGAGCAUCCC | |
| R4263_ | CUUUCAAGACUAAUAGAUUGCUCCUUACGAGGAG | 1030 |
| CasPhi12 | ACUCUUCGCCCAGAGCAUCCCA | |
| R4264_ | CUUUCAAGACUAAUAGAUUGCUCCUUACGAGGAG | 1031 |
| CasPhi12 | ACGCCCAGAGCAUCCCAUGGAA | |
| R4265_ | CUUUCAAGACUAAUAGAUUGCUCCUUACGAGGAG | 1032 |
| CasPhi12 | ACCAUGGGAUGCUCUGGGCGAA | |
| R4266_ | CUUUCAAGACUAAUAGAUUGCUCCUUACGAGGAG | 1033 |
| CasPhi12 | ACGCUCCAGGUUCCAUGGGAUG | |
| R4267_ | CUUUCAAGACUAAUAGAUUGCUCCUUACGAGGAG | 1034 |
| CasPhi12 | ACUCCCAGCAUGGCACCAGACA | |
| R4268_ | CUUUCAAGACUAAUAGAUUGCUCCUUACGAGGAG | 1035 |
| CasPhi12 | ACCUCUGUCUGGUGCCAUGCUG | |
| R4269_ | CUUUCAAGACUAAUAGAUUGCUCCUUACGAGGAG | 1036 |
| CasPhi12 | ACGAUACCAGCAUCCAGGGUGC | |
| R4270_ | CUUUCAAGACUAAUAGAUUGCUCCUUACGAGGAG | 1037 |
| CasPhi12 | ACAGGGCAGGGUCACCAUCACC | |
| R4271_ | CUUUCAAGACUAAUAGAUUGCUCCUUACGAGGAG | 1038 |
| CasPhi12 | ACAAGUCGGUGAUGGUGACCCU | |
| R4272_ | CUUUCAAGACUAAUAGAUUGCUCCUUACGAGGAG | 1039 |
| CasPhi12 | ACAACAGCGUGCCGGAGGAGGA | |
| R4273_ | CUUUCAAGACUAAUAGAUUGCUCCUUACGAGGAG | 1040 |
| CasPhi12 | ACGCCACACCAGCAUCCCGGCC | |
| R4274_ | CUUUCAAGACUAAUAGAUUGCUCCUUACGAGGAG | 1041 |
| CasPhi12 | ACAGCACACGCAGGCUGUGCAG | |
| R4275_ | CUUUCAAGACUAAUAGAUUGCUCCUUACGAGGAG | 1042 |
| CasPhi12 | ACACAGUUGAGCACACGCAGGC | |
| R4276_ | CUUUCAAGACUAAUAGAUUGCUCCUUACGAGGAG | 1043 |
| CasPhi12 | ACCCUUGACAGUUGAGCACACG | |
| R4277_ | CUUUCAAGACUAAUAGAUUGCUCCUUACGAGGAG | 1044 |
| CasPhi12 | ACGCUGACUCUUCCGAAUAAAC | |
| R4278_ | CUUUCAAGACUAAUAGAUUGCUCCUUACGAGGAG | 1045 |
| CasPhi12 | ACAUUCGGAAGAGUCAGCUAAU | |
| R4279_ | CUUUCAAGACUAAUAGAUUGCUCCUUACGAGGAG | 1046 |
| CasPhi12 | ACUUCGGAAGAGUCAGCUAAUC | |
| R4280_ | CUUUCAAGACUAAUAGAUUGCUCCUUACGAGGAG | 1047 |
| CasPhi12 | ACGGAAGAGUCAGCUAAUCCAG | |
| R4281_ | CUUUCAAGACUAAUAGAUUGCUCCUUACGAGGAG | 1048 |
| CasPhi12 | ACUGCUGCCCCUGGCCGGUGGG | |
| R4282_ | CUUUCAAGACUAAUAGAUUGCUCCUUACGAGGAG | 1049 |
| CasPhi12 | ACAGGAUGCGGCUAUACCCACC | |
| R4283_ | CUUUCAAGACUAAUAGAUUGCUCCUUACGAGGAG | 1050 |
| CasPhi12 | ACCCAGCUGCUGCAACCAGCAC | |
| R4284_ | CUUUCAAGACUAAUAGAUUGCUCCUUACGAGGAG | 1051 |
| CasPhi12 | ACCAGCAGCUGGGAACUUCCGG | |
| R4285_ | CUUUCAAGACUAAUAGAUUGCUCCUUACGAGGAG | 1052 |
| CasPhi12 | ACCGGGACGACGCCUGCCUCUA | |
| R4286_ | CUUUCAAGACUAAUAGAUUGCUCCUUACGAGGAG | 1053 |
| CasPhi12 | ACGUGGCCCCGACUGUGAUGAC | |
| R4287_ | CUUUCAAGACUAAUAGAUUGCUCCUUACGAGGAG | 1054 |
| CasPhi12 | ACCCUUGGGGACUUUGGGGACU | |
| R4288_ | CUUUCAAGACUAAUAGAUUGCUCCUUACGAGGAG | 1055 |
| CasPhi12 | ACGUCCCCAAAGUCCCCAAGGU | |
| R4289_ | CUUUCAAGACUAAUAGAUUGCUCCUUACGAGGAG | 1056 |
| CasPhi12 | ACGGGACUUUGGGGACUAAUUU | |
| R4290_ | CUUUCAAGACUAAUAGAUUGCUCCUUACGAGGAG | 1057 |
| CasPhi12 | ACGGGGACUAAUUUUGGACGCU | |
| R4291_ | CUUUCAAGACUAAUAGAUUGCUCCUUACGAGGAG | 1058 |
| CasPhi12 | ACGGGACUAAUUUUGGACGCUG | |
| R4292_ | CUUUCAAGACUAAUAGAUUGCUCCUUACGAGGAG | 1059 |
| CasPhi12 | ACUGGACGCUGUGUGGAUCUCU | |
| R4293_ | CUUUCAAGACUAAUAGAUUGCUCCUUACGAGGAG | 1060 |
| CasPhi12 | ACGGACGCUGUGUGGAUCUCUU | |
| R4294_ | CUUUCAAGACUAAUAGAUUGCUCCUUACGAGGAG | 1061 |
| CasPhi12 | ACGACGCUGUGUGGAUCUCUUU | |
| R4295_ | CUUUCAAGACUAAUAGAUUGCUCCUUACGAGGAG | 1062 |
| CasPhi12 | ACCCGGGGGCAAAGAGAUCCAC | |
| R4296_ | CUUUCAAGACUAAUAGAUUGCUCCUUACGAGGAG | 1063 |
| CasPhi12 | ACGCCCCCGGGAAGGACAUCAU | |
| R4297_ | CUUUCAAGACUAAUAGAUUGCUCCUUACGAGGAG | 1064 |
| CasPhi12 | ACCCCCCGGGAAGGACAUCAUC | |
| R4298_ | CUUUCAAGACUAAUAGAUUGCUCCUUACGAGGAG | 1065 |
| CasPhi12 | ACAUGUCACAGAGUGGGACCUC | |
| R4299_ | CUUUCAAGACUAAUAGAUUGCUCCUUACGAGGAG | 1066 |
| CasPhi12 | ACUGGCUCGGAUGCUGAGCCGG | |
| R4300_ | CUUUCAAGACUAAUAGAUUGCUCCUUACGAGGAG | 1067 |
| CasPhi12 | ACCCCUGGCCGAGCUGCGGCAG | |
| R4301_ | CUUUCAAGACUAAUAGAUUGCUCCUUACGAGGAG | 1068 |
| CasPhi12 | ACGUAGAGAAGUGGAUCAGCCU | |
| R4302_ | CUUUCAAGACUAAUAGAUUGCUCCUUACGAGGAG | 1069 |
| CasPhi12 | ACGGUAGAGAAGUGGAUCAGCC | |
| R4303_ | CUUUCAAGACUAAUAGAUUGCUCCUUACGAGGAG | 1070 |
| CasPhi12 | ACUCUACCAAAGACGUCAUCAA | |
| R4304_ | CUUUCAAGACUAAUAGAUUGCUCCUUACGAGGAG | 1071 |
| CasPhi12 | ACAUGACGUCUUUGGUAGAGAA | |
| R4305_ | CUUUCAAGACUAAUAGAUUGCUCCUUACGAGGAG | 1072 |
| CasPhi12 | ACCCUGAGGACCAGCAGGUGCU | |
| R4306_ | CUUUCAAGACUAAUAGAUUGCUCCUUACGAGGAG | 1073 |
| CasPhi12 | ACGGGGUCAGCACCUGCUGGUC | |
| R4307_ | CUUUCAAGACUAAUAGAUUGCUCCUUACGAGGAG | 1074 |
| CasPhi12 | ACGAGUGGGCCCCGAGUGUGCC | |
| R4308_ | CUUUCAAGACUAAUAGAUUGCUCCUUACGAGGAG | 1075 |
| CasPhi12 | ACUGGGGCACAGCGGGCUGUAG | |
| R4309_ | CUUUCAAGACUAAUAGAUUGCUCCUUACGAGGAG | 1076 |
| CasPhi12 | ACUCCAGGAGCGGGAGGCGUCG | |
| R4310_ | CUUUCAAGACUAAUAGAUUGCUCCUUACGAGGAG | 1077 |
| CasPhi12 | ACCAGACCUGCUGGCCUCCUAU | |
| R4311_ | CUUUCAAGACUAAUAGAUUGCUCCUUACGAGGAG | 1078 |
| CasPhi12 | ACAGGGCCUUGCAGACCUGCUG | |
| R4312_ | CUUUCAAGACUAAUAGAUUGCUCCUUACGAGGAG | 1079 |
| CasPhi12 | ACGGGGGUGAGGGUGUCUAUGC | |
| R4313_ | CUUUCAAGACUAAUAGAUUGCUCCUUACGAGGAG | 1080 |
| CasPhi12 | ACGGGGUGAGGGUGUCUAUGCC | |
| R4314_ | CUUUCAAGACUAAUAGAUUGCUCCUUACGAGGAG | 1081 |
| CasPhi12 | ACGCACGGGGAACCAGGCAGCA | |
| R4315_ | CUUUCAAGACUAAUAGAUUGCUCCUUACGAGGAG | 1082 |
| CasPhi12 | ACCCCGUGCCAACUGCAGCAUC | |
| R4316_ | CUUUCAAGACUAAUAGAUUGCUCCUUACGAGGAG | 1083 |
| CasPhi12 | ACUGGAUGCUGCAGUUGGCACG | |
| R4317_ | CUUUCAAGACUAAUAGAUUGCUCCUUACGAGGAG | 1084 |
| CasPhi12 | ACUGGUGGCAGUGGACAUGGGU | |
| R4318_ | CUUUCAAGACUAAUAGAUUGCUCCUUACGAGGAG | 1085 |
| CasPhi12 | ACCACUUCCCAAUGGAAGCUGC | |
| R4319_ | CUUUCAAGACUAAUAGAUUGCUCCUUACGAGGAG | 1086 |
| CasPhi12 | ACCAUUGGGAAGUGGAAGACCU | |
| R4320_ | CUUUCAAGACUAAUAGAUUGCUCCUUACGAGGAG | 1087 |
| CasPhi12 | ACGGAAGUGGAAGACCUUAGUG | |
| R4321_ | CUUUCAAGACUAAUAGAUUGCUCCUUACGAGGAG | 1088 |
| CasPhi12 | ACGUGUCCGGAGGCAGCCUGCG | |
| R4322_ | CUUUCAAGACUAAUAGAUUGCUCCUUACGAGGAG | 1089 |
| CasPhi12 | ACGCCACCAGGCGGCCAGUGUC | |
| R4323_ | CUUUCAAGACUAAUAGAUUGCUCCUUACGAGGAG | 1090 |
| CasPhi12 | ACCUGCUGCCAUGCCCCAGGGC | |
| R4324_ | CUUUCAAGACUAAUAGAUUGCUCCUUACGAGGAG | 1091 |
| CasPhi12 | ACCAGCCCUGGGGCAUGGCAGC | |
| R4325_ | CUUUCAAGACUAAUAGAUUGCUCCUUACGAGGAG | 1092 |
| CasPhi12 | ACCAUUCCAGCCCUGGGGCAUG | |
| R4326_ | CUUUCAAGACUAAUAGAUUGCUCCUUACGAGGAG | 1093 |
| CasPhi12 | ACGCAUUCCAGCCCUGGGGCAU | |
| R4327_ | CUUUCAAGACUAAUAGAUUGCUCCUUACGAGGAG | 1094 |
| CasPhi12 | ACUGCAUUCCAGCCCUGGGGCA | |
| R4328_ | CUUUCAAGACUAAUAGAUUGCUCCUUACGAGGAG | 1095 |
| CasPhi12 | ACAUUUUGCAUUCCAGCCCUGG | |
| R4329_ | CUUUCAAGACUAAUAGAUUGCUCCUUACGAGGAG | 1096 |
| CasPhi12 | ACCAUCCAGUCAGGGUCCAUCC | |
| R4330_ | CUUUCAAGACUAAUAGAUUGCUCCUUACGAGGAG | 1097 |
| CasPhi12 | ACUCCACGCUGUAGGCUCCCAG | |
| R4331_ | CUUUCAAGACUAAUAGAUUGCUCCUUACGAGGAG | 1098 |
| CasPhi12 | ACCCACACACAGGUUGUCCACG | |
| R4332_ | CUUUCAAGACUAAUAGAUUGCUCCUUACGAGGAG | 1099 |
| CasPhi12 | ACUCCACUGGUCCUGUCUGCUC | |
| R4333_ | CUUUCAAGACUAAUAGAUUGCUCCUUACGAGGAG | 1100 |
| CasPhi12 | ACCUGAAGGCCGGCUCCGGCAG | |

TABLE R
CasΦ.32 gRNAs targeting mouse PCSK9

| | | SEQ |
|---|---|---|
| | Repeat + spacer sequence | ID |
| Name | RNA Sequence (5′ → 3′) | NO |
| R4238_ | GCUGGGGACCGAUCCUGAUUGCUCGCUGCGGCGAG | 1101 |
| CasPhi32 | ACCCGCUGUUGCCGCCGCUGCU | |
| R4239_ | GCUGGGGACCGAUCCUGAUUGCUCGCUGCGGCGAG | 1102 |
| CasPhi32 | ACCCGCCGCUGCUGCUGCUGUU | |
| R4240_ | GCUGGGGACCGAUCCUGAUUGCUCGCUGCGGCGAG | 1103 |
| CasPhi32 | ACCUGCUACUGUGCCCCACCGG | |
| R4241_ | GCUGGGGACCGAUCCUGAUUGCUCGCUGCGGCGAG | 1104 |
| CasPhi32 | ACAUAAUCUCCAUCCUCGUCCU | |
| R4242_ | GCUGGGGACCGAUCCUGAUUGCUCGCUGCGGCGAG | 1105 |
| CasPhi32 | ACUGAAGAGCUGAUGCUCGCCC | |
| R4243_ | GCUGGGGACCGAUCCUGAUUGCUCGCUGCGGCGAG | 1106 |
| CasPhi32 | ACGAGCAACGGCGGAAGGUGGC | |
| R4244_ | GCUGGGGACCGAUCCUGAUUGCUCGCUGCGGCGAG | 1107 |
| CasPhi32 | ACCUGGCAGCCUCCAGGCCUCC | |
| R4245_ | GCUGGGGACCGAUCCUGAUUGCUCGCUGCGGCGAG | 1108 |
| CasPhi32 | ACUGGUGCUGAUGGAGGAGACC | |
| R4246_ | GCUGGGGACCGAUCCUGAUUGCUCGCUGCGGCGAG | 1109 |
| CasPhi32 | ACAAUCUGUAGCCUCUGGGUCU | |
| R4247_ | GCUGGGGACCGAUCCUGAUUGCUCGCUGCGGCGAG | 1110 |
| CasPhi32 | ACUUCAAUCUGUAGCCUCUGGG | |
| R4248_ | GCUGGGGACCGAUCCUGAUUGCUCGCUGCGGCGAG | 1111 |
| CasPhi32 | ACGUUCAAUCUGUAGCCUCUGG | |
| R4249_ | GCUGGGGACCGAUCCUGAUUGCUCGCUGCGGCGAG | 1112 |
| CasPhi32 | ACAACAAACUGCCCACCGCCUG | |
| R4250_ | GCUGGGGACCGAUCCUGAUUGCUCGCUGCGGCGAG | 1113 |
| CasPhi32 | ACAUGACAUAGCCCCGGCGGGC | |
| R4251_ | GCUGGGGACCGAUCCUGAUUGCUCGCUGCGGCGAG | 1114 |
| CasPhi32 | ACUACAUAUCUUUUAUGACCUC | |
| R4252_ | GCUGGGGACCGAUCCUGAUUGCUCGCUGCGGCGAG | 1115 |
| CasPhi32 | ACUAUGACCUCUUCCCUGGCUU | |
| R4253_ | GCUGGGGACCGAUCCUGAUUGCUCGCUGCGGCGAG | 1116 |
| CasPhi32 | ACAUGACCUCUUCCCUGGCUUC | |
| R4254_ | GCUGGGGACCGAUCCUGAUUGCUCGCUGCGGCGAG | 1117 |
| CasPhi32 | ACUGACCUCUUCCCUGGCUUCU | |
| R4255_ | GCUGGGGACCGAUCCUGAUUGCUCGCUGCGGCGAG | 1118 |
| CasPhi32 | ACACCAAGAAGCCAGGGAAGAG | |
| R4256_ | GCUGGGGACCGAUCCUGAUUGCUCGCUGCGGCGAG | 1119 |
| CasPhi32 | ACCCUGGCUUCUUGGUGAAGAU | |
| R4257_ | GCUGGGGACCGAUCCUGAUUGCUCGCUGCGGCGAG | 1120 |
| CasPhi32 | ACUUGGUGAAGAUGAGCAGUGA | |
| R4258_ | GCUGGGGACCGAUCCUGAUUGCUCGCUGCGGCGAG | 1121 |
| CasPhi32 | ACGUGAAGAUGAGCAGUGACCU | |
| R4259_ | GCUGGGGACCGAUCCUGAUUGCUCGCUGCGGCGAG | 1122 |
| CasPhi32 | ACCCCCAUGUGGAGUACAUUGA | |
| R4260_ | GCUGGGGACCGAUCCUGAUUGCUCGCUGCGGCGAG | 1123 |
| CasPhi32 | ACCUCAAUGUACUCCACAUGGG | |
| R4261_ | GCUGGGGACCGAUCCUGAUUGCUCGCUGCGGCGAG | 1124 |
| CasPhi32 | ACAGGAAGACUCCUUUGUCUUC | |
| R4262_ | GCUGGGGACCGAUCCUGAUUGCUCGCUGCGGCGAG | 1125 |
| CasPhi32 | ACGUCUUCGCCCAGAGCAUCCC | |
| R4263_ | GCUGGGGACCGAUCCUGAUUGCUCGCUGCGGCGAG | 1126 |
| CasPhi32 | ACUCUUCGCCCAGAGCAUCCCA | |
| R4264_ | GCUGGGGACCGAUCCUGAUUGCUCGCUGCGGCGAG | 1127 |
| CasPhi32 | ACGCCCAGAGCAUCCCAUGGAA | |
| R4265_ | GCUGGGGACCGAUCCUGAUUGCUCGCUGCGGCGAG | 1128 |
| CasPhi32 | ACCAUGGGAUGCUCUGGGCGAA | |
| R4266_ | GCUGGGGACCGAUCCUGAUUGCUCGCUGCGGCGAG | 1129 |
| CasPhi32 | ACGCUCCAGGUUCCAUGGGAUG | |
| R4267_ | GCUGGGGACCGAUCCUGAUUGCUCGCUGCGGCGAG | 1130 |
| CasPhi32 | ACUCCCAGCAUGGCACCAGACA | |
| R4268_ | GCUGGGGACCGAUCCUGAUUGCUCGCUGCGGCGAG | 1131 |
| CasPhi32 | ACCUCUGUCUGGUGCCAUGCUG | |
| R4269_ | GCUGGGGACCGAUCCUGAUUGCUCGCUGCGGCGAG | 1132 |
| CasPhi32 | ACGAUACCAGCAUCCAGGGUGC | |
| R4270_ | GCUGGGGACCGAUCCUGAUUGCUCGCUGCGGCGAG | 1133 |
| CasPhi32 | ACAGGGCAGGGUCACCAUCACC | |
| R4271_ | GCUGGGGACCGAUCCUGAUUGCUCGCUGCGGCGAG | 1134 |
| CasPhi32 | ACAAGUCGGUGAUGGUGACCCU | |
| R4272_ | GCUGGGGACCGAUCCUGAUUGCUCGCUGCGGCGAG | 1135 |
| CasPhi32 | ACAACAGCGUGCCGGAGGAGGA | |
| R4273_ | GCUGGGGACCGAUCCUGAUUGCUCGCUGCGGCGAG | 1136 |
| CasPhi32 | ACGCCACACCAGCAUCCCGGCC | |
| R4274_ | GCUGGGGACCGAUCCUGAUUGCUCGCUGCGGCGAG | 1137 |
| CasPhi32 | ACAGCACACGCAGGCUGUGCAG | |
| R4275_ | GCUGGGGACCGAUCCUGAUUGCUCGCUGCGGCGAG | 1138 |
| CasPhi32 | ACACAGUUGAGCACACGCAGGC | |
| R4276_ | GCUGGGGACCGAUCCUGAUUGCUCGCUGCGGCGAG | 1139 |
| CasPhi32 | ACCCUUGACAGUUGAGCACACG | |
| R4277_ | GCUGGGGACCGAUCCUGAUUGCUCGCUGCGGCGAG | 1140 |
| CasPhi32 | ACGCUGACUCUUCCGAAUAAAC | |
| R4278_ | GCUGGGGACCGAUCCUGAUUGCUCGCUGCGGCGAG | 1141 |
| CasPhi32 | ACAUUCGGAAGAGUCAGCUAAU | |
| R4279_ | GCUGGGGACCGAUCCUGAUUGCUCGCUGCGGCGAG | 1142 |
| CasPhi32 | ACUUCGGAAGAGUCAGCUAAUC | |
| R4280_ | GCUGGGGACCGAUCCUGAUUGCUCGCUGCGGCGAG | 1143 |
| CasPhi32 | ACGGAAGAGUCAGCUAAUCCAG | |
| R4281_ | GCUGGGGACCGAUCCUGAUUGCUCGCUGCGGCGAG | 1144 |
| CasPhi32 | ACUGCUGCCCCUGGCCGGUGGG | |
| R4282_ | GCUGGGGACCGAUCCUGAUUGCUCGCUGCGGCGAG | 1145 |
| CasPhi32 | ACAGGAUGCGGCUAUACCCACC | |
| R4283_ | GCUGGGGACCGAUCCUGAUUGCUCGCUGCGGCGAG | 1146 |
| CasPhi32 | ACCCAGCUGCUGCAACCAGCAC | |
| R4284_ | GCUGGGGACCGAUCCUGAUUGCUCGCUGCGGCGAG | 1147 |
| CasPhi32 | ACCAGCAGCUGGGAACUUCCGG | |
| R4285_ | GCUGGGGACCGAUCCUGAUUGCUCGCUGCGGCGAG | 1148 |
| CasPhi32 | ACCGGGACGACGCCUGCCUCUA | |
| R4286_ | GCUGGGGACCGAUCCUGAUUGCUCGCUGCGGCGAG | 1149 |
| CasPhi32 | ACGUGGCCCCGACUGUGAUGAC | |
| R4287_ | GCUGGGGACCGAUCCUGAUUGCUCGCUGCGGCGAG | 1150 |
| CasPhi32 | ACCCUUGGGGACUUUGGGGACU | |
| R4288_ | GCUGGGGACCGAUCCUGAUUGCUCGCUGCGGCGAG | 1151 |
| CasPhi32 | ACGUCCCCAAAGUCCCCAAGGU | |
| R4289_ | GCUGGGGACCGAUCCUGAUUGCUCGCUGCGGCGAG | 1152 |
| CasPhi32 | ACGGGACUUUGGGGACUAAUUU | |
| R4290_ | GCUGGGGACCGAUCCUGAUUGCUCGCUGCGGCGAG | 1153 |
| CasPhi32 | ACGGGGACUAAUUUUGGACGCU | |
| R4291_ | GCUGGGGACCGAUCCUGAUUGCUCGCUGCGGCGAG | 1154 |
| CasPhi32 | ACGGGACUAAUUUUGGACGCUG | |
| R4292_ | GCUGGGGACCGAUCCUGAUUGCUCGCUGCGGCGAG | 1155 |
| CasPhi32 | ACUGGACGCUGUGUGGAUCUCU | |
| R4293_ | GCUGGGGACCGAUCCUGAUUGCUCGCUGCGGCGAG | 1156 |
| CasPhi32 | ACGGACGCUGUGUGGAUCUCUU | |
| R4294_ | GCUGGGGACCGAUCCUGAUUGCUCGCUGCGGCGAG | 1157 |
| CasPhi32 | ACGACGCUGUGUGGAUCUCUUU | |
| R4295_ | GCUGGGGACCGAUCCUGAUUGCUCGCUGCGGCGAG | 1158 |
| CasPhi32 | ACCCGGGGGCAAAGAGAUCCAC | |
| R4296_ | GCUGGGGACCGAUCCUGAUUGCUCGCUGCGGCGAG | 1159 |
| CasPhi32 | ACGCCCCCGGGAAGGACAUCAU | |
| R4297_ | GCUGGGGACCGAUCCUGAUUGCUCGCUGCGGCGAG | 1160 |
| CasPhi32 | ACCCCCCGGGAAGGACAUCAUC | |
| R4298_ | GCUGGGGACCGAUCCUGAUUGCUCGCUGCGGCGAG | 1161 |
| CasPhi32 | ACAUGUCACAGAGUGGGACCUC | |
| R4299_ | GCUGGGGACCGAUCCUGAUUGCUCGCUGCGGCGAG | 1162 |
| CasPhi32 | ACUGGCUCGGAUGCUGAGCCGG | |
| R4300_ | GCUGGGGACCGAUCCUGAUUGCUCGCUGCGGCGAG | 1163 |
| CasPhi32 | ACCCCUGGCCGAGCUGCGGCAG | |
| R4301_ | GCUGGGGACCGAUCCUGAUUGCUCGCUGCGGCGAG | 1164 |
| CasPhi32 | ACGUAGAGAAGUGGAUCAGCCU | |
| R4302_ | GCUGGGGACCGAUCCUGAUUGCUCGCUGCGGCGAG | 1165 |
| CasPhi32 | ACGGUAGAGAAGUGGAUCAGCC | |
| R4303_ | GCUGGGGACCGAUCCUGAUUGCUCGCUGCGGCGAG | 1166 |
| CasPhi32 | ACUCUACCAAAGACGUCAUCAA | |
| R4304_ | GCUGGGGACCGAUCCUGAUUGCUCGCUGCGGCGAG | 1167 |
| CasPhi32 | ACAUGACGUCUUUGGUAGAGAA | |
| R4305_ | GCUGGGGACCGAUCCUGAUUGCUCGCUGCGGCGAG | 1168 |
| CasPhi32 | ACCCUGAGGACCAGCAGGUGCU | |
| R4306_ | GCUGGGGACCGAUCCUGAUUGCUCGCUGCGGCGAG | 1169 |
| CasPhi32 | ACGGGGUCAGCACCUGCUGGUC | |
| R4307_ | GCUGGGGACCGAUCCUGAUUGCUCGCUGCGGCGAG | 1170 |
| CasPhi32 | ACGAGUGGGCCCCGAGUGUGCC | |
| R4308_ | GCUGGGGACCGAUCCUGAUUGCUCGCUGCGGCGAG | 1171 |
| CasPhi32 | ACUGGGGCACAGCGGGCUGUAG | |
| R4309_ | GCUGGGGACCGAUCCUGAUUGCUCGCUGCGGCGAG | 1172 |
| CasPhi32 | ACUCCAGGAGCGGGAGGCGUCG | |
| R4310_ | GCUGGGGACCGAUCCUGAUUGCUCGCUGCGGCGAG | 1173 |
| CasPhi32 | ACCAGACCUGCUGGCCUCCUAU | |
| R4311_ | GCUGGGGACCGAUCCUGAUUGCUCGCUGCGGCGAG | 1174 |
| CasPhi32 | ACAGGGCCUUGCAGACCUGCUG | |
| R4312_ | GCUGGGGACCGAUCCUGAUUGCUCGCUGCGGCGAG | 1175 |
| CasPhi32 | ACGGGGGUGAGGGUGUCUAUGC | |
| R4313_ | GCUGGGGACCGAUCCUGAUUGCUCGCUGCGGCGAG | 1176 |
| CasPhi32 | ACGGGGUGAGGGUGUCUAUGCC | |
| R4314_ | GCUGGGGACCGAUCCUGAUUGCUCGCUGCGGCGAG | 1177 |
| CasPhi32 | ACGCACGGGGAACCAGGCAGCA | |
| R4315_ | GCUGGGGACCGAUCCUGAUUGCUCGCUGCGGCGAG | 1178 |
| CasPhi32 | ACCCCGUGCCAACUGCAGCAUC | |
| R4316_ | GCUGGGGACCGAUCCUGAUUGCUCGCUGCGGCGAG | 1179 |
| CasPhi32 | ACUGGAUGCUGCAGUUGGCACG | |
| R4317_ | GCUGGGGACCGAUCCUGAUUGCUCGCUGCGGCGAG | 1180 |
| CasPhi32 | ACUGGUGGCAGUGGACAUGGGU | |
| R4318_ | GCUGGGGACCGAUCCUGAUUGCUCGCUGCGGCGAG | 1181 |
| CasPhi32 | ACCACUUCCCAAUGGAAGCUGC | |
| R4319_ | GCUGGGGACCGAUCCUGAUUGCUCGCUGCGGCGAG | 1182 |
| CasPhi32 | ACCAUUGGGAAGUGGAAGACCU | |
| R4320_ | GCUGGGGACCGAUCCUGAUUGCUCGCUGCGGCGAG | 1183 |
| CasPhi32 | ACGGAAGUGGAAGACCUUAGUG | |
| R4321_ | GCUGGGGACCGAUCCUGAUUGCUCGCUGCGGCGAG | 1184 |
| CasPhi32 | ACGUGUCCGGAGGCAGCCUGCG | |
| R4322_ | GCUGGGGACCGAUCCUGAUUGCUCGCUGCGGCGAG | 1185 |
| CasPhi32 | ACGCCACCAGGCGGCCAGUGUC | |
| R4323_ | GCUGGGGACCGAUCCUGAUUGCUCGCUGCGGCGAG | 1186 |
| CasPhi32 | ACCUGCUGCCAUGCCCCAGGGC | |
| R4324_ | GCUGGGGACCGAUCCUGAUUGCUCGCUGCGGCGAG | 1187 |
| CasPhi32 | ACCAGCCCUGGGGCAUGGCAGC | |
| R4325_ | GCUGGGGACCGAUCCUGAUUGCUCGCUGCGGCGAG | 1188 |
| CasPhi32 | ACCAUUCCAGCCCUGGGGCAUG | |
| R4326_ | GCUGGGGACCGAUCCUGAUUGCUCGCUGCGGCGAG | 1189 |
| CasPhi32 | ACGCAUUCCAGCCCUGGGGCAU | |
| R4327_ | GCUGGGGACCGAUCCUGAUUGCUCGCUGCGGCGAG | 1190 |
| CasPhi32 | ACUGCAUUCCAGCCCUGGGGCA | |
| R4328_ | GCUGGGGACCGAUCCUGAUUGCUCGCUGCGGCGAG | 1191 |
| CasPhi32 | ACAUUUUGCAUUCCAGCCCUGG | |
| R4329_ | GCUGGGGACCGAUCCUGAUUGCUCGCUGCGGCGAG | 1192 |
| CasPhi32 | ACCAUCCAGUCAGGGUCCAUCC | |
| R4330_ | GCUGGGGACCGAUCCUGAUUGCUCGCUGCGGCGAG | 1193 |
| CasPhi32 | ACUCCACGCUGUAGGCUCCCAG | |
| R4331_ | GCUGGGGACCGAUCCUGAUUGCUCGCUGCGGCGAG | 1194 |
| CasPhi32 | ACCCACACACAGGUUGUCCACG | |
| R4332_ | GCUGGGGACCGAUCCUGAUUGCUCGCUGCGGCGAG | 1195 |
| CasPhi32 | ACUCCACUGGUCCUGUCUGCUC | |
| R4333_ | GCUGGGGACCGAUCCUGAUUGCUCGCUGCGGCGAG | 1196 |
| CasPhi32 | ACCUGAAGGCCGGCUCCGGCAG | |

TABLE S
CasΦ.12 gRNAs targeting Bak1 in CHO cells

| | Repeat + spacer RNA Sequence (5′ → 3′), | |
|---|---|---|
| Name | shown as DNA | SEQ ID NO |
| R2452 | CTTTCAAGACTAATAGATTGCTCCTTACGAG | 1197 |
| Bak1_CasPhi12_1 | GAGACGAAGCTATGTTTTCCATCTC | |
| R2453 | CTTTCAAGACTAATAGATTGCTCCTTACGAG | 1198 |
| Bak1_CasPhi12_2 | GAGACGCAGGGGCAGCCGCCCCCTG | |
| R2454 | CTTTCAAGACTAATAGATTGCTCCTTACGAG | 1199 |
| Bak1_CasPhi12_3 | GAGACCTCCTAGAACCCAACAGGTA | |
| R2455 | CTTTCAAGACTAATAGATTGCTCCTTACGAG | 1200 |
| Bak1_CasPhi12_4 | GAGACGAAAGACCTCCTCTGTGTCC | |
| R2456 | CTTTCAAGACTAATAGATTGCTCCTTACGAG | 1201 |
| Bak1_CasPhi12_5 | GAGACTCCATCTCGGGGTTGGCAGG | |
| R2457 | CTTTCAAGACTAATAGATTGCTCCTTACGAG | 1202 |
| Bak1_CasPhi12_6 | GAGACTTCCTGATGGTGGAGATGGA | |
| R2849_Bak1_ | CTTTCAAGACTAATAGATTGCTCCTTACGAG | 1203 |
| CasPhi12_nsd_sg1 | GAGACCTGACTCCCAGCTCTGACCC | |
| R2850_Bak1_ | CTTTCAAGACTAATAGATTGCTCCTTACGAG | 1204 |
| CasPhi12_nsd_sg2 | GAGACTGGGGTCAGAGCTGGGAGTC | |
| R2851_Bak1__ | CTTTCAAGACTAATAGATTGCTCCTTACGAG | 1205 |
| CasPhi12_nsd_sg3 | GAGACGAAAGACCTCCTCTGTGTCC | |
| R2852_Bak1_ | CTTTCAAGACTAATAGATTGCTCCTTACGAG | 1206 |
| CasPhi12_nsd_sg4 | GAGACCGAAGCTATGTTTTCCATCT | |
| R2853_Bak1_ | CTTTCAAGACTAATAGATTGCTCCTTACGAG | 1207 |
| CasPhi12_nsd_sg5 | GAGACGAAGCTATGTTTTCCATCTC | |
| R2854_Bak1_ | CTTTCAAGACTAATAGATTGCTCCTTACGAG | 1208 |
| CasPhi12_nsd_sg6 | GAGACTCCATCTCCACCATCAGGAA | |
| R2855_Bak1 | CTTTCAAGACTAATAGATTGCTCCTTACGAG | 1209 |
| CasPhi12_nsd_sg7 | GAGACCCATCTCCACCATCAGGAAC | |
| R2856_Bak1_ | CTTTCAAGACTAATAGATTGCTCCTTACGAG | 1210 |
| CasPhi12_nsd_sg8 | GAGACCTGATGGTGGAGATGGAAAA | |
| R2857_Bak1_ | CTTTCAAGACTAATAGATTGCTCCTTACGAG | 1211 |
| CasPhi12_nsd_sg9 | GAGACCATCTCCACCATCAGGAACA | |
| R2858_Bak1_ | CTTTCAAGACTAATAGATTGCTCCTTACGAG | 1212 |
| CasPhi12_nsd_sg10 | GAGACTTCCTGATGGTGGAGATGGA | |
| R2859_Bak1_ | CTTTCAAGACTAATAGATTGCTCCTTACGAG | 1213 |
| CasPhi12_nsd_sg11 | GAGACGCAGGGGCAGCCGCCCCCTG | |
| R2860_Bak1_ | CTTTCAAGACTAATAGATTGCTCCTTACGAG | 1214 |
| CasPhi12_nsd_sg12 | GAGACTCCATCTCGGGGTTGGCAGG | |
| R2861_Bak1_ | CTTTCAAGACTAATAGATTGCTCCTTACGAG | 1215 |
| CasPhi12_nsd_sg13 | GAGACTAGGAGCAAATTGTCCATCT | |
| R2862_Bak1_ | CTTTCAAGACTAATAGATTGCTCCTTACGAG | 1216 |
| CasPhi12_nsd_sg14 | GAGACGGTTCTAGGAGCAAATTGTC | |
| R2863_Bak1_ | CTTTCAAGACTAATAGATTGCTCCTTACGAG | 1217 |
| CasPhi12_nsd_sg15 | GAGACGCTCCTAGAACCCAACAGGT | |
| R2864_Bak1_ | CTTTCAAGACTAATAGATTGCTCCTTACGAG | 1218 |
| CasPhi12_nsd_sg16 | GAGACCTCCTAGAACCCAACAGGTA | |
| R3977_Bak1_ | CTTTCAAGACTAATAGATTGCTCCTTACGAG | 1219 |
| CasPhi12_exon1_sg1 | GAGACTCCAGACGCCATCTTTCAGG | |
| R3978_Bak1_ | CTTTCAAGACTAATAGATTGCTCCTTACGAG | 1220 |
| CasPhi12_exon1_sg2 | GAGACTGGTAAGAGTCCTCCTGCCC | |
| R3979_Bak1_ | CTTTCAAGACTAATAGATTGCTCCTTACGAG | 1221 |
| CasPhi12_exon3_sg1 | GAGACTTACAGCATCTTGGGTCAGG | |
| R3980_Bak1_ | CTTTCAAGACTAATAGATTGCTCCTTACGAG | 1222 |
| CasPhi12_exon3_sg2 | GAGACGGTCAGGTGGGCCGGCAGCT | |
| R3981_Bak1_ | CTTTCAAGACTAATAGATTGCTCCTTACGAG | 1223 |
| CasPhi12_exon3_sg3 | GAGACCTATCATTGGAGATGACATT | |
| R3982_Bak1_ | CTTTCAAGACTAATAGATTGCTCCTTACGAG | 1224 |
| CasPhi12_exon3_sg4 | GAGACGAGATGACATTAACCGGAGA | |
| R3983_Bak1_ | CTTTCAAGACTAATAGATTGCTCCTTACGAG | 1225 |
| CasPhi12_exon3_sg5 | GAGACTGGAACTCTGTGTCGTATCT | |
| R3984_Bak1_ | CTTTCAAGACTAATAGATTGCTCCTTACGAG | 1226 |
| CasPhi12_exon3_sg6 | GAGACCAGAATTTACTGGAGCAGCT | |
| R3985_Bak1_ | CTTTCAAGACTAATAGATTGCTCCTTACGAG | 1227 |
| CasPhi12_exon3_sg7 | GAGACACTGGAGCAGCTGCAGCCCA | |
| R3986_Bak1_ | CTTTCAAGACTAATAGATTGCTCCTTACGAG | 1228 |
| CasPhi12_exon3_sg8 | GAGACCCAGCTGTGGGCTGCAGCTG | |
| R3987_Bak1_ | CTTTCAAGACTAATAGATTGCTCCTTACGAG | 1229 |
| CasPhi12_exon3_sg9 | GAGACGTAGGCATTCCCAGCTGTGG | |
| R3988_Bak1_ | CTTTCAAGACTAATAGATTGCTCCTTACGAG | 1230 |
| CasPhi12_exon3_ | GAGACGTGAAGAGTTCGTAGGCATT | |
| sg10 | | |
| R3989_Bak1_ | CTTTCAAGACTAATAGATTGCTCCTTACGAG | 1231 |
| CasPhi12_exon3_ | GAGACACCAAGATTGCCTCCAGGTA | |
| sg11 | | |
| R3990_Bak1_ | CTTTCAAGACTAATAGATTGCTCCTTACGAG | 1232 |
| CasPhi12_exon3_ | GAGACCCTCCAGGTACCCACCACCA | |
| sg12 | | |

TABLE T
CasΦ.32 gRNAs targeting Bak1 in CHO cells

| | Repeat + spacer RNA Sequence (5′ → 3′), | |
|---|---|---|
| Name | shown as DNA | SEQ ID NO |
| R2452 | GCTGGGGACCGATCCTGATTGCTCGCTGCGG | 1233 |
| Bak1_CasPhi32_1 | CGAGACGAAGCTATGTTTTCCATCTC | |
| R2453 | GCTGGGGACCGATCCTGATTGCTCGCTGCGG | 1234 |
| Bak1_CasPhi32_2 | CGAGACGCAGGGGCAGCCGCCCCCTG | |
| R2454 | GCTGGGGACCGATCCTGATTGCTCGCTGCGG | 1235 |
| Bak1_CasPhi32_3 | CGAGACCTCCTAGAACCCAACAGGTA | |
| R2455 | GCTGGGGACCGATCCTGATTGCTCGCTGCGG | 1236 |
| Bak1_CasPhi32_4 | CGAGACGAAAGACCTCCTCTGTGTCC | |
| R2456 | GCTGGGGACCGATCCTGATTGCTCGCTGCGG | 1237 |
| Bak1_CasPhi32_5 | CGAGACTCCATCTCGGGGTTGGCAGG | |
| R2457 | GCTGGGGACCGATCCTGATTGCTCGCTGCGG | 1238 |
| Bak1_CasPhi32_6 | CGAGACTTCCTGATGGTGGAGATGGA | |
| R2849_Bak1_ | GCTGGGGACCGATCCTGATTGCTCGCTGCGG | 1239 |
| CasPhi32_nsd_sg1 | CGAGACCTGACTCCCAGCTCTGACCC | |
| R2850_Bak1_ | GCTGGGGACCGATCCTGATTGCTCGCTGCGG | 1240 |
| CasPhi32_nsd_sg2 | CGAGACTGGGGTCAGAGCTGGGAGTC | |
| R2851_Bak1_ | GCTGGGGACCGATCCTGATTGCTCGCTGCGG | 1241 |
| CasPhi32_nsd_sg3 | CGAGACGAAAGACCTCCTCTGTGTCC | |
| R2852_Bak1_ | GCTGGGGACCGATCCTGATTGCTCGCTGCGG | 1242 |
| CasPhi32_nsd_sg4 | CGAGACCGAAGCTATGTTTTCCATCT | |
| R2853_Bak1_ | GCTGGGGACCGATCCTGATTGCTCGCTGCGG | 1243 |
| CasPhi32_nsd_sg5 | CGAGACGAAGCTATGTTTTCCATCTC | |
| R2854_Bak1_ | GCTGGGGACCGATCCTGATTGCTCGCTGCGG | 1244 |
| CasPhi32_nsd_sg6 | CGAGACTCCATCTCCACCATCAGGAA | |
| R2855_Bak1_ | GCTGGGGACCGATCCTGATTGCTCGCTGCGG | 1245 |
| CasPhi32_nsd_sg7 | CGAGACCCATCTCCACCATCAGGAAC | |
| R2856_Bak1_ | GCTGGGGACCGATCCTGATTGCTCGCTGCGG | 1246 |
| CasPhi32_nsd_sg8 | CGAGACCTGATGGTGGAGATGGAAAA | |
| R2857_Bak1_ | GCTGGGGACCGATCCTGATTGCTCGCTGCGG | 1247 |
| CasPhi32_nsd_sg9 | CGAGACCATCTCCACCATCAGGAACA | |
| R2858_Bak1_ | GCTGGGGACCGATCCTGATTGCTCGCTGCGG | 1248 |
| CasPhi32_nsd_sg10 | CGAGACTTCCTGATGGTGGAGATGGA | |
| R2859_Bak1_ | GCTGGGGACCGATCCTGATTGCTCGCTGCGG | 1249 |
| CasPhi32_nsd_sg11 | CGAGACGCAGGGGCAGCCGCCCCCTG | |
| R2860_Bak1_ | GCTGGGGACCGATCCTGATTGCTCGCTGCGG | 1250 |
| CasPhi32_nsd_sg12 | CGAGACTCCATCTCGGGGTTGGCAGG | |
| R2861_Bak1_ | GCTGGGGACCGATCCTGATTGCTCGCTGCGG | 1251 |
| CasPhi32_nsd_sg13 | CGAGACTAGGAGCAAATTGTCCATCT | |
| R2862_Bak1_ | GCTGGGGACCGATCCTGATTGCTCGCTGCGG | 1252 |
| CasPhi32_nsd_sg14 | CGAGACGGTTCTAGGAGCAAATTGTC | |
| R2863_Bak1_ | GCTGGGGACCGATCCTGATTGCTCGCTGCGG | 1253 |
| CasPhi32_nsd_sg15 | CGAGACGCTCCTAGAACCCAACAGGT | |
| R2864_Bak1_ | GCTGGGGACCGATCCTGATTGCTCGCTGCGG | 1254 |
| CasPhi32_nsd_sg16 | CGAGACCTCCTAGAACCCAACAGGTA | |
| R3977_Bak1_ | GCTGGGGACCGATCCTGATTGCTCGCTGCGG | 1255 |
| CasPhi32_exon1_sg1 | CGAGACTCCAGACGCCATCTTTCAGG | |
| R3978_Bak1_ | GCTGGGGACCGATCCTGATTGCTCGCTGCGG | 1256 |
| CasPhi32_exon1_sg2 | CGAGACTGGTAAGAGTCCTCCTGCCC | |
| R3979_Bak1_ | GCTGGGGACCGATCCTGATTGCTCGCTGCGG | 1257 |
| CasPhi32_exon3_sg1 | CGAGACTTACAGCATCTTGGGTCAGG | |
| R3980_Bak1_ | GCTGGGGACCGATCCTGATTGCTCGCTGCGG | 1258 |
| CasPhi32_exon3_sg2 | CGAGACGGTCAGGTGGGCCGGCAGCT | |
| R3981_Bak1_ | GCTGGGGACCGATCCTGATTGCTCGCTGCGG | 1259 |
| CasPhi32_exon3_sg3 | CGAGACCTATCATTGGAGATGACATT | |
| R3982_Bak1_ | GCTGGGGACCGATCCTGATTGCTCGCTGCGG | 1260 |
| CasPhi32_exon3_sg4 | CGAGACGAGATGACATTAACCGGAGA | |
| R3983_Bak1_ | GCTGGGGACCGATCCTGATTGCTCGCTGCGG | 1261 |
| CasPhi32_exon3_sg5 | CGAGACTGGAACTCTGTGTCGTATCT | |
| R3984_Bak1_ | GCTGGGGACCGATCCTGATTGCTCGCTGCGG | 1262 |
| CasPhi32_exon3_sg6 | CGAGACCAGAATTTACTGGAGCAGCT | |
| R3985_Bak1_ | GCTGGGGACCGATCCTGATTGCTCGCTGCGG | 1263 |
| CasPhi32_exon3_sg7 | CGAGACACTGGAGCAGCTGCAGCCCA | |
| R3986_Bak1_ | GCTGGGGACCGATCCTGATTGCTCGCTGCGG | 1264 |
| CasPhi32_exon3_sg8 | CGAGACCCAGCTGTGGGCTGCAGCTG | |
| R3987_Bak1_ | GCTGGGGACCGATCCTGATTGCTCGCTGCGG | 1265 |
| CasPhi32_exon3_sg9 | CGAGACGTAGGCATTCCCAGCTGTGG | |
| R3988_Bak1_ | GCTGGGGACCGATCCTGATTGCTCGCTGCGG | 1266 |
| CasPhi32_exon3_ | CGAGACGTGAAGAGTTCGTAGGCATT | |
| sg10 | | |
| R3989_Bak1_ | GCTGGGGACCGATCCTGATTGCTCGCTGCGG | 1267 |
| CasPhi32_exon3_ | CGAGACACCAAGATTGCCTCCAGGTA | |
| sg11 | | |
| R3990_Bak1_ | GCTGGGGACCGATCCTGATTGCTCGCTGCGG | 1268 |
| CasPhi32_exon3_ | CGAGACCCTCCAGGTACCCACCACCA | |
| sg12 | | |

TABLE U
CasΦ.12 gRNAs targeting Bax in CHO cells

| | Repeat + spacer RNA Sequence (5′ → 3′), | |
|---|---|---|
| Name | shown as DNA | SEQ ID NO |
| R2458 | CTTTCAAGACTAATAGATTGCTCCTTACGAG | 1269 |
| Bax_CasPhi12_1 | GAGACCTAATGTGGATACTAACTCC | |
| R2459 | CTTTCAAGACTAATAGATTGCTCCTTACGAG | 1270 |
| Bax_CasPhi12_2 | GAGACTTCCGTGTGGCAGCTGACAT | |
| R2460 | CTTTCAAGACTAATAGATTGCTCCTTACGAG | 1271 |
| Bax_CasPhi12_3 | GAGACCTGATGGCAACTTCAACTGG | |
| R2461 | CTTTCAAGACTAATAGATTGCTCCTTACGAG | 1272 |
| Bax_CasPhi12_4 | GAGACTACTTTGCTAGCAAACTGGT | |
| R2462 | CTTTCAAGACTAATAGATTGCTCCTTACGAG | 1273 |
| Bax_CasPhi12_5 | GAGACAGCACCAGTTTGCTAGCAAA | |
| R2463 | CTTTCAAGACTAATAGATTGCTCCTTACGAG | 1274 |
| Bax_CasPhi12_6 | GAGACAACTGGGGCCGGGTTGTTGC | |
| R2865_Bax_ | CTTTCAAGACTAATAGATTGCTCCTTACGAG | 1275 |
| CasPhi12_nsd_sg1 | GAGACTTCTCTTTCCTGTAGGATGA | |
| R2866_Bax_ | CTTTCAAGACTAATAGATTGCTCCTTACGAG | 1276 |
| CasPhi12_nsd_sg2 | GAGACTCTTTCCTGTAGGATGATTG | |
| R2867_Bax_ | CTTTCAAGACTAATAGATTGCTCCTTACGAG | 1277 |
| CasPhi12_nsd_sg3 | GAGACCCTGTAGGATGATTGCTAAT | |
| R2868_Bax_ | CTTTCAAGACTAATAGATTGCTCCTTACGAG | 1278 |
| CasPhi12_nsd_sg4 | GAGACCTGTAGGATGATTGCTAATG | |
| R2869_Bax_ | CTTTCAAGACTAATAGATTGCTCCTTACGAG | 1279 |
| CasPhi12_nsd_sg5 | GAGACCTAATGTGGATACTAACTCC | |
| R2870_Bax_ | CTTTCAAGACTAATAGATTGCTCCTTACGAG | 1280 |
| CasPhi12_nsd_sg6 | GAGACTTCCGTGTGGCAGCTGACAT | |
| R2871_Bax_ | CTTTCAAGACTAATAGATTGCTCCTTACGAG | 1281 |
| CasPhi12_nsd_sg7 | GAGACCGTGTGGCAGCTGACATGTT | |
| R2872_Bax_ | CTTTCAAGACTAATAGATTGCTCCTTACGAG | 1282 |
| CasPhi12_nsd_sg8 | GAGACCCATCAGCAAACATGTCAGC | |
| R2873_Bax_ | CTTTCAAGACTAATAGATTGCTCCTTACGAG | 1283 |
| CasPhi12_nsd_sg9 | GAGACAAGTTGCCATCAGCAAACAT | |
| R2874_Bax_ | CTTTCAAGACTAATAGATTGCTCCTTACGAG | 1284 |
| CasPhi12_nsd_sg10 | GAGACGCTGATGGCAACTTCAACTG | |
| R2875_Bax_ | CTTTCAAGACTAATAGATTGCTCCTTACGAG | 1285 |
| CasPhi12_nsd_sg11 | GAGACCTGATGGCAACTTCAACTGG | |
| R2876_Bax_ | CTTTCAAGACTAATAGATTGCTCCTTACGAG | 1286 |
| CasPhi12_nsd_sg12 | GAGACAACTGGGGCCGGGTTGTTGC | |
| R2877_Bax_ | CTTTCAAGACTAATAGATTGCTCCTTACGAG | 1287 |
| CasPhi12_nsd_sg13 | GAGACTTGCCCTTTTCTACTTTGCT | |
| R2878_Bax_ | CTTTCAAGACTAATAGATTGCTCCTTACGAG | 1288 |
| CasPhi12_nsd_sg14 | GAGACCCCTTTTCTACTTTGCTAGC | |
| R2879_Bax_ | CTTTCAAGACTAATAGATTGCTCCTTACGAG | 1289 |
| CasPhi12_nsd_sg15 | GAGACCTAGCAAAGTAGAAAAGGGC | |
| R2880_Bax_ | CTTTCAAGACTAATAGATTGCTCCTTACGAG | 1290 |
| CasPhi12_nsd_sg16 | GAGACGCTAGCAAAGTAGAAAAGGG | |
| R2881_Bax_ | CTTTCAAGACTAATAGATTGCTCCTTACGAG | 1291 |
| CasPhi12_nsd_sg17 | GAGACTCTACTTTGCTAGCAAACTG | |
| R2882_Bax_ | CTTTCAAGACTAATAGATTGCTCCTTACGAG | 1292 |
| CasPhi12_nsd_sg18 | GAGACCTACTTTGCTAGCAAACTGG | |
| R2883_Bax_ | CTTTCAAGACTAATAGATTGCTCCTTACGAG | 1293 |
| CasPhi12_nsd_sg19 | GAGACTACTTTGCTAGCAAACTGGT | |
| R2884_Bax_ | CTTTCAAGACTAATAGATTGCTCCTTACGAG | 1294 |
| CasPhi12_nsd_sg20 | GAGACGCTAGCAAACTGGTGCTCAA | |
| R2885_Bax_ | CTTTCAAGACTAATAGATTGCTCCTTACGAG | 1295 |
| CasPhi12_nsd_sg21 | GAGACCTAGCAAACTGGTGCTCAAG | |
| R2886_Bax_ | CTTTCAAGACTAATAGATTGCTCCTTACGAG | 1296 |
| CasPhi12_nsd_sg22 | GAGACAGCACCAGTTTGCTAGCAAA | |

TABLE V
CasΦ.32 gRNAs targeting Bax in CHO cells

| | Repeat + spacer RNA Sequence (5′ → 3′), | |
|---|---|---|
| Name | shown as DNA | SEQ ID NO |
| R2458 | GCTGGGGACCGATCCTGATTGCTCGCTGCG | 1297 |
| Bax CasPhi32_1 | GCGAGACCTAATGTGGATACTAACTCC | |
| R2459 | GCTGGGGACCGATCCTGATTGCTCGCTGCG | 1298 |
| Bax CasPhi32_2 | GCGAGACTTCCGTGTGGCAGCTGACAT | |
| R2460 | GCTGGGGACCGATCCTGATTGCTCGCTGCG | 1299 |
| Bax CasPhi32_3 | GCGAGACCTGATGGCAACTTCAACTGG | |
| R2461 | GCTGGGGACCGATCCTGATTGCTCGCTGCG | 1300 |
| Bax CasPhi32_4 | GCGAGACTACTTTGCTAGCAAACTGGT | |
| R2462 | GCTGGGGACCGATCCTGATTGCTCGCTGCG | 1301 |
| Bax CasPhi32_5 | GCGAGACAGCACCAGTTTGCTAGCAAA | |
| R2463 | GCTGGGGACCGATCCTGATTGCTCGCTGCG | 1302 |
| Bax CasPhi32_6 | GCGAGACAACTGGGGCCGGGTTGTTGC | |
| R2865_Bax_ | GCTGGGGACCGATCCTGATTGCTCGCTGCG | 1303 |
| CasPhi32_nsd_sg1 | GCGAGACTTCTCTTTCCTGTAGGATGA | |
| R2866_Bax_ | GCTGGGGACCGATCCTGATTGCTCGCTGCG | 1304 |
| CasPhi32_nsd_sg2 | GCGAGACTCTTTCCTGTAGGATGATTG | |
| R2867_Bax_ | GCTGGGGACCGATCCTGATTGCTCGCTGCG | 1305 |
| CasPhi32_nsd_sg3 | GCGAGACCCTGTAGGATGATTGCTAAT | |
| R2868_Bax_ | GCTGGGGACCGATCCTGATTGCTCGCTGCG | 1306 |
| CasPhi32_nsd_sg4 | GCGAGACCTGTAGGATGATTGCTAATG | |
| R2869_Bax_ | GCTGGGGACCGATCCTGATTGCTCGCTGCG | 1307 |
| CasPhi32_nsd_sg5 | GCGAGACCTAATGTGGATACTAACTCC | |
| R2870_Bax_ | GCTGGGGACCGATCCTGATTGCTCGCTGCG | 1308 |
| CasPhi32_nsd_sg6 | GCGAGACTTCCGTGTGGCAGCTGACAT | |
| R2871_Bax_ | GCTGGGGACCGATCCTGATTGCTCGCTGCG | 1309 |
| CasPhi32_nsd_sg7 | GCGAGACCGTGTGGCAGCTGACATGTT | |
| R2872_Bax_ | GCTGGGGACCGATCCTGATTGCTCGCTGCG | 1310 |
| CasPhi32_nsd_sg8 | GCGAGACCCATCAGCAAACATGTCAGC | |
| R2873_Bax_ | GCTGGGGACCGATCCTGATTGCTCGCTGCG | 1311 |
| CasPhi32_nsd_sg9 | GCGAGACAAGTTGCCATCAGCAAACAT | |
| R2874_Bax_ | GCTGGGGACCGATCCTGATTGCTCGCTGCG | 1312 |
| CasPhi32_nsd_sg10 | GCGAGACGCTGATGGCAACTTCAACTG | |
| R2875_Bax_ | GCTGGGGACCGATCCTGATTGCTCGCTGCG | 1313 |
| CasPhi32_nsd_sg11 | GCGAGACCTGATGGCAACTTCAACTGG | |
| R2876_Bax_ | GCTGGGGACCGATCCTGATTGCTCGCTGCG | 1314 |
| CasPhi32_nsd_sg12 | GCGAGACAACTGGGGCCGGGTTGTTGC | |
| R2877_Bax_ | GCTGGGGACCGATCCTGATTGCTCGCTGCG | 1315 |
| CasPhi32_nsd_sg13 | GCGAGACTTGCCCTTTTCTACTTTGCT | |
| R2878_Bax_ | GCTGGGGACCGATCCTGATTGCTCGCTGCG | 1316 |
| CasPhi32_nsd_sg14 | GCGAGACCCCTTTTCTACTTTGCTAGC | |
| R2879_Bax_ | GCTGGGGACCGATCCTGATTGCTCGCTGCG | 1317 |
| CasPhi32_nsd_sg15 | GCGAGACCTAGCAAAGTAGAAAAGGGC | |
| R2880_Bax_ | GCTGGGGACCGATCCTGATTGCTCGCTGCG | 1318 |
| CasPhi32_nsd_sg16 | GCGAGACGCTAGCAAAGTAGAAAAGGG | |
| R2881_Bax_ | GCTGGGGACCGATCCTGATTGCTCGCTGCG | 1319 |
| CasPhi32_nsd_sg17 | GCGAGACTCTACTTTGCTAGCAAACTG | |
| R2882_Bax_ | GCTGGGGACCGATCCTGATTGCTCGCTGCG | 1320 |
| CasPhi32_nsd_sg18 | GCGAGACCTACTTTGCTAGCAAACTGG | |
| R2883_Bax_ | GCTGGGGACCGATCCTGATTGCTCGCTGCG | 1321 |
| CasPhi32_nsd_sg19 | GCGAGACTACTTTGCTAGCAAACTGGT | |
| R2884_Bax_ | GCTGGGGACCGATCCTGATTGCTCGCTGCG | 1322 |
| CasPhi32_nsd_sg20 | GCGAGACGCTAGCAAACTGGTGCTCAA | |
| R2885_Bax_ | GCTGGGGACCGATCCTGATTGCTCGCTGCG | 1323 |
| CasPhi32_nsd_sg21 | GCGAGACCTAGCAAACTGGTGCTCAAG | |
| R2886_Bax_ | GCTGGGGACCGATCCTGATTGCTCGCTGCG | 1324 |
| CasPhi32_nsd_sg22 | GCGAGACAGCACCAGTTTGCTAGCAAA | |

TABLE W
CasΦ.12 gRNAs targeting_Fut8 in CHO cells

| | Repeat + spacer RNA Sequence (5′ → 3′), | |
|---|---|---|
| Name | shown as DNA | SEQ ID NO |
| R2464 | CTTTCAAGACTAATAGATTGCTCCTTACGAG | 1325 |
| Fut8_CasPhi12_1 | GAGACCCACTTTGTCAGTGCGTCTG | |
| R2465 | CTTTCAAGACTAATAGATTGCTCCTTACGAG | 1326 |
| Fut8_CasPhi12_2 | GAGACCTCAATGGGATGGAAGGCTG | |
| R2466 | CTTTCAAGACTAATAGATTGCTCCTTACGAG | 1327 |
| Fut8_CasPhi12_3 | GAGACAGGAATACATGGTACACGTT | |
| R2467 | CTTTCAAGACTAATAGATTGCTCCTTACGAG | 1328 |
| Fut8_CasPhi12_4 | GAGACAAGAACATTTTCAGCTTCTC | |
| R2468 | CTTTCAAGACTAATAGATTGCTCCTTACGAG | 1329 |
| Fut8_CasPhi12_5 | GAGACATCCACTTTCATTCTGCGTT | |
| R2469 | CTTTCAAGACTAATAGATTGCTCCTTACGAG | 1330 |
| Fut8_CasPhi12_6 | GAGACTTTGTTAAAGGAGGCAAAGA | |
| R2887_Fut8__ | CTTTCAAGACTAATAGATTGCTCCTTACGAG | 1331 |
| CasPhi12_nsd_sg1 | GAGACTCCCCAGAGTCCATGTCAGA | |
| R2888_Fut8_ | CTTTCAAGACTAATAGATTGCTCCTTACGAG | 1332 |
| CasPhi12_nsd_sg2 | GAGACTCAGTGCGTCTGACATGGAC | |
| R2889_Fut8__ | CTTTCAAGACTAATAGATTGCTCCTTACGAG | 1333 |
| CasPhi12_nsd_sg3 | GAGACGTCAGTGCGTCTGACATGGA | |
| R2890_Fut8_ | CTTTCAAGACTAATAGATTGCTCCTTACGAG | 1334 |
| CasPhi12_nsd_sg4 | GAGACCCACTTTGTCAGTGCGTCTG | |
| R2891_Fut8_ | CTTTCAAGACTAATAGATTGCTCCTTACGAG | 1335 |
| CasPhi12_nsd_sg5 | GAGACTGTTCCCACTTTGTCAGTGC | |
| R2892_Fut8_ | CTTTCAAGACTAATAGATTGCTCCTTACGAG | 1336 |
| CasPhi12_nsd_sg6 | GAGACCTCAATGGGATGGAAGGCTG | |
| R2893_Fut8_ | CTTTCAAGACTAATAGATTGCTCCTTACGAG | 1337 |
| CasPhi12_nsd_sg7 | GAGACCATCCCATTGAGGAATACAT | |
| R2894_Fut8_ | CTTTCAAGACTAATAGATTGCTCCTTACGAG | 1338 |
| CasPhi12_nsd_sg8 | GAGACAGGAATACATGGTACACGTT | |
| R2895_Fut8_ | CTTTCAAGACTAATAGATTGCTCCTTACGAG | 1339 |
| CasPhi12_nsd_sg9 | GAGACAACGTGTACCATGTATTCCT | |
| R2896_Fut8_ | CTTTCAAGACTAATAGATTGCTCCTTACGAG | 1340 |
| CasPhi12_nsd_sg10 | GAGACTTCAACGTGTACCATGTATT | |
| R2897_Fut8_ | CTTTCAAGACTAATAGATTGCTCCTTACGAG | 1341 |
| CasPhi12_nsd_sg11 | GAGACAAGAACATTTTCAGCTTCTC | |
| R2898_Fut8_ | CTTTCAAGACTAATAGATTGCTCCTTACGAG | 1342 |
| CasPhi12_nsd_sg12 | GAGACGAGAAGCTGAAAATGTTCTT | |
| R2899_Fut8_ | CTTTCAAGACTAATAGATTGCTCCTTACGAG | 1343 |
| CasPhi12_nsd_sg13 | GAGACTCAGCTTCTCGAACGCAGAA | |
| R2900_Fut8_ | CTTTCAAGACTAATAGATTGCTCCTTACGAG | 1344 |
| CasPhi12_nsd_sg14 | GAGACCAGCTTCTCGAACGCAGAAT | |
| R2901_Fut8_ | CTTTCAAGACTAATAGATTGCTCCTTACGAG | 1345 |
| CasPhi12_nsd_sg15 | GAGACTGCGTTCGAGAAGCTGAAAA | |
| R2902_Fut8_ | CTTTCAAGACTAATAGATTGCTCCTTACGAG | 1346 |
| CasPhi12_nsd_sg16 | GAGACAGCTTCTCGAACGCAGAATG | |
| R2903_Fut8_ | CTTTCAAGACTAATAGATTGCTCCTTACGAG | 1347 |
| CasPhi12_nsd_sg17 | GAGACATTCTGCGTTCGAGAAGCTG | |
| R2904_Fut8_ | CTTTCAAGACTAATAGATTGCTCCTTACGAG | 1348 |
| CasPhi12_nsd_sg18 | GAGACCATTCTGCGTTCGAGAAGCT | |
| R2905_Fut8_ | CTTTCAAGACTAATAGATTGCTCCTTACGAG | 1349 |
| CasPhi12_nsd_sg19 | GAGACTCGAACGCAGAATGAAAGTG | |
| R2906_Fut8_ | CTTTCAAGACTAATAGATTGCTCCTTACGAG | 1350 |
| CasPhi12_nsd_sg20 | GAGACATCCACTTTCATTCTGCGTT | |
| R2907_Fut8_ | CTTTCAAGACTAATAGATTGCTCCTTACGAG | 1351 |
| CasPhi12_nsd_sg21 | GAGACTATCCACTTTCATTCTGCGT | |
| R2908_Fut8_ | CTTTCAAGACTAATAGATTGCTCCTTACGAG | 1352 |
| CasPhi12_nsd_sg22 | GAGACTTATCCACTTTCATTCTGCG | |
| R2909_Fut8_ | CTTTCAAGACTAATAGATTGCTCCTTACGAG | 1353 |
| CasPhi12_nsd_sg23 | GAGACTTTATCCACTTTCATTCTGC | |
| R2910_Fut8_ | CTTTCAAGACTAATAGATTGCTCCTTACGAG | 1354 |
| CasPhi12_nsd_sg24 | GAGACTTTTATCCACTTTCATTCTG | |
| R2911_Fut8_ | CTTTCAAGACTAATAGATTGCTCCTTACGAG | 1355 |
| CasPhi12_nsd_sg25 | GAGACAACAAAGAAGGGTCATCAGT | |
| R2912_Fut8_ | CTTTCAAGACTAATAGATTGCTCCTTACGAG | 1356 |
| CasPhi12_nsd_sg26 | GAGACCCTCCTTTAACAAAGAAGGG | |
| R2913_Fut8_ | CTTTCAAGACTAATAGATTGCTCCTTACGAG | 1357 |
| CasPhi12_nsd_sg27 | GAGACGCCTCCTTTAACAAAGAAGG | |
| R2914_Fut8_ | CTTTCAAGACTAATAGATTGCTCCTTACGAG | 1358 |
| CasPhi12_nsd_sg28 | GAGACTTTGTTAAAGGAGGCAAAGA | |
| R2915_Fut8_ | CTTTCAAGACTAATAGATTGCTCCTTACGAG | 1359 |
| CasPhi12_nsd_sg29 | GAGACGTTAAAGGAGGCAAAGACAA | |
| R2916_Fut8_ | CTTTCAAGACTAATAGATTGCTCCTTACGAG | 1360 |
| CasPhi12_nsd_sg30 | GAGACTTAAAGGAGGCAAAGACAAA | |
| R2917_Fut8_ | CTTTCAAGACTAATAGATTGCTCCTTACGAG | 1361 |
| CasPhi12_nsd_sg31 | GAGACTCTTTGCCTCCTTTAACAAA | |
| R2918_Fut8_ | CTTTCAAGACTAATAGATTGCTCCTTACGAG | 1362 |
| CasPhi12_nsd_sg32 | GAGACGTCTTTGCCTCCTTTAACAA | |
| R2919_Fut8_ | CTTTCAAGACTAATAGATTGCTCCTTACGAG | 1363 |
| CasPhi12_nsd_sg33 | GAGACGTCTAACTTACTTTGTCTTT | |
| R2920_Fut8_ | CTTTCAAGACTAATAGATTGCTCCTTACGAG | 1364 |
| CasPhi12_nsd_sg34 | GAGACTTGGTCTAACTTACTTTGTC | |

TABLE X
CasΦ.32 gRNAs targeting_Fut8 in CHO cells

| | Repeat + spacer RNA Sequence (5′ → 3′), | |
|---|---|---|
| Name | shown as DNA | SEQ ID NO |
| R2464 | GCTGGGGACCGATCCTGATTGCTCGCTGCG | 1365 |
| Fut8_CasPhi32_1 | GCGAGACCCACTTTGTCAGTGCGTCTG | |
| R2465 | GCTGGGGACCGATCCTGATTGCTCGCTGCG | 1366 |
| Fut8_CasPhi32_2 | GCGAGACCTCAATGGGATGGAAGGCTG | |
| R2466 | GCTGGGGACCGATCCTGATTGCTCGCTGCG | 1367 |
| Fut8_CasPhi32_3 | GCGAGACAGGAATACATGGTACACGTT | |
| R2467 | GCTGGGGACCGATCCTGATTGCTCGCTGCG | 1368 |
| Fut8_CasPhi32_4 | GCGAGACAAGAACATTTTCAGCTTCTC | |
| R2468 | GCTGGGGACCGATCCTGATTGCTCGCTGCG | 1369 |
| Fut8_CasPhi32_5 | GCGAGACATCCACTTTCATTCTGCGTT | |
| R2469 | GCTGGGGACCGATCCTGATTGCTCGCTGCG | 1370 |
| Fut8_CasPhi32_6 | GCGAGACTTTGTTAAAGGAGGCAAAGA | |
| R2887_Fut8__ | GCTGGGGACCGATCCTGATTGCTCGCTGCG | 1371 |
| CasPhi32_nsd_sg1 | GCGAGACTCCCCAGAGTCCATGTCAGA | |
| R2888_Fut8_ | GCTGGGGACCGATCCTGATTGCTCGCTGCG | 1372 |
| CasPhi32_nsd_sg2 | GCGAGACTCAGTGCGTCTGACATGGAC | |
| R2889_Fut8_ | GCTGGGGACCGATCCTGATTGCTCGCTGCG | 1373 |
| CasPhi32_nsd_sg3 | GCGAGACGTCAGTGCGTCTGACATGGA | |
| R2890_Fut8_ | GCTGGGGACCGATCCTGATTGCTCGCTGCG | 1374 |
| CasPhi32_nsd_sg4 | GCGAGACCCACTTTGTCAGTGCGTCTG | |
| R2891_Fut8_ | GCTGGGGACCGATCCTGATTGCTCGCTGCG | 1375 |
| CasPhi32_nsd_sg5 | GCGAGACTGTTCCCACTTTGTCAGTGC | |
| R2892_Fut8_ | GCTGGGGACCGATCCTGATTGCTCGCTGCG | 1376 |
| CasPhi32_nsd_sg6 | GCGAGACCTCAATGGGATGGAAGGCTG | |
| R2893_Fut8_ | GCTGGGGACCGATCCTGATTGCTCGCTGCG | 1377 |
| CasPhi32_nsd_sg7 | GCGAGACCATCCCATTGAGGAATACAT | |
| R2894_Fut8_ | GCTGGGGACCGATCCTGATTGCTCGCTGCG | 1378 |
| CasPhi32_nsd_sg8 | GCGAGACAGGAATACATGGTACACGTT | |
| R2895_Fut8_ | GCTGGGGACCGATCCTGATTGCTCGCTGCG | 1379 |
| CasPhi32_nsd_sg9 | GCGAGACAACGTGTACCATGTATTCCT | |
| R2896_Fut8_ | GCTGGGGACCGATCCTGATTGCTCGCTGCG | 1380 |
| CasPhi32_nsd_sg10 | GCGAGACTTCAACGTGTACCATGTATT | |
| R2897_Fut8_ | GCTGGGGACCGATCCTGATTGCTCGCTGCG | 1381 |
| CasPhi32_nsd_sg11 | GCGAGACAAGAACATTTTCAGCTTCTC | |
| R2898_Fut8_ | GCTGGGGACCGATCCTGATTGCTCGCTGCG | 1382 |
| CasPhi32_nsd_sg12 | GCGAGACGAGAAGCTGAAAATGTTCTT | |
| R2899_Fut8_ | GCTGGGGACCGATCCTGATTGCTCGCTGCG | 1383 |
| CasPhi32_nsd_sg13 | GCGAGACTCAGCTTCTCGAACGCAGAA | |
| R2900_Fut8_ | GCTGGGGACCGATCCTGATTGCTCGCTGCG | 1384 |
| CasPhi32_nsd_sg14 | GCGAGACCAGCTTCTCGAACGCAGAAT | |
| R2901_Fut8_ | GCTGGGGACCGATCCTGATTGCTCGCTGCG | 1385 |
| CasPhi32_nsd_sg15 | GCGAGACTGCGTTCGAGAAGCTGAAAA | |
| R2902_Fut8_ | GCTGGGGACCGATCCTGATTGCTCGCTGCG | 1386 |
| CasPhi32_nsd_sg16 | GCGAGACAGCTTCTCGAACGCAGAATG | |
| R2903_Fut8_ | GCTGGGGACCGATCCTGATTGCTCGCTGCG | 1387 |
| CasPhi32_nsd_sg17 | GCGAGACATTCTGCGTTCGAGAAGCTG | |
| R2904_Fut8_ | GCTGGGGACCGATCCTGATTGCTCGCTGCG | 1388 |
| CasPhi32_nsd_sg18 | GCGAGACCATTCTGCGTTCGAGAAGCT | |
| R2905_Fut8 | GCTGGGGACCGATCCTGATTGCTCGCTGCG | 1389 |
| CasPhi32_nsd_sg19 | GCGAGACTCGAACGCAGAATGAAAGTG | |
| R2906_Fut8_ | GCTGGGGACCGATCCTGATTGCTCGCTGCG | 1390 |
| CasPhi32_ | GCGAGACATCCACTTTCATTCTGCGTT | |
| CasPhi32_nsd_sg20 | | |
| R2907_Fut8_ | GCTGGGGACCGATCCTGATTGCTCGCTGCG | 1391 |
| CasPhi32_nsd_sg21 | GCGAGACTATCCACTTTCATTCTGCGT | |
| R2908_Fut8_ | GCTGGGGACCGATCCTGATTGCTCGCTGCG | 1392 |
| CasPhi32_nsd_sg22 | GCGAGACTTATCCACTTTCATTCTGCG | |
| R2909_Fut8_ | GCTGGGGACCGATCCTGATTGCTCGCTGCG | 1393 |
| CasPhi32_nsd_sg23 | GCGAGACTTTATCCACTTTCATTCTGC | |
| R2910_Fut8_ | GCTGGGGACCGATCCTGATTGCTCGCTGCG | 1394 |
| CasPhi32_nsd_sg24 | GCGAGACTTTTATCCACTTTCATTCTG | |
| R2911_Fut8_ | GCTGGGGACCGATCCTGATTGCTCGCTGCG | 1395 |
| CasPhi32_nsd_sg25 | GCGAGACAACAAAGAAGGGTCATCAGT | |
| R2912_Fut8_ | GCTGGGGACCGATCCTGATTGCTCGCTGCG | 1396 |
| CasPhi32_nsd_sg26 | GCGAGACCCTCCTTTAACAAAGAAGGG | |
| R2913_Fut8_ | GCTGGGGACCGATCCTGATTGCTCGCTGCG | 1397 |
| CasPhi32_nsd_sg27 | GCGAGACGCCTCCTTTAACAAAGAAGG | |
| R2914_Fut8_ | GCTGGGGACCGATCCTGATTGCTCGCTGCG | 1398 |
| CasPhi32_nsd_sg28 | GCGAGACTTTGTTAAAGGAGGCAAAGA | |
| R2915_Fut8_ | GCTGGGGACCGATCCTGATTGCTCGCTGCG | 1399 |
| CasPhi32_nsd_sg29 | GCGAGACGTTAAAGGAGGCAAAGACAA | |
| R2916_Fut8_ | GCTGGGGACCGATCCTGATTGCTCGCTGCG | 1400 |
| CasPhi32_nsd_sg30 | GCGAGACTTAAAGGAGGCAAAGACAAA | |
| R2917_Fut8_ | GCTGGGGACCGATCCTGATTGCTCGCTGCG | 1401 |
| CasPhi32_nsd_sg31 | GCGAGACTCTTTGCCTCCTTTAACAAA | |
| R2918_Fut8_ | GCTGGGGACCGATCCTGATTGCTCGCTGCG | 1402 |
| CasPhi32_nsd_sg32 | GCGAGACGTCTTTGCCTCCTTTAACAA | |
| R2919_Fut8_ | GCTGGGGACCGATCCTGATTGCTCGCTGCG | 1403 |
| CasPhi32_nsd_sg33 | GCGAGACGTCTAACTTACTTTGTCTTT | |
| R2920_Fut8_ | GCTGGGGACCGATCCTGATTGCTCGCTGCG | 1404 |
| CasPhi32_nsd_sg34 | GCGAGACTTGGTCTAACTTACTTTGTC | |

TABLE Y
CasΦ.12 gRNAs targeting human TRAC in T cells

| | Repeat + spacer RNA Sequence (5′ → 3′), | |
|---|---|---|
| Name | shown as DNA | SEQ ID NO |
| R3040_CasPhi12_S | ATTGCTCCTTACGAGGAGACTGGATATCTGT | 1533 |
| | GGGACA | |
| R3041_CasPhi12_S | ATTGCTCCTTACGAGGAGACTCCCACAGATA | 1534 |
| | TCCAGA | |
| R3042_CasPhi12_S | ATTGCTCCTTACGAGGAGACGAGTCTCTCAG | 1535 |
| | CTGGTA | |
| R3043_CasPhi12_S | ATTGCTCCTTACGAGGAGACAGAGTCTCTCA | 1536 |
| | GCTGGT | |
| R3044_CasPhi12_S | ATTGCTCCTTACGAGGAGACTCACTGGATTT | 1537 |
| | AGAGTC | |
| R3045_CasPhi12_S | ATTGCTCCTTACGAGGAGACAGAATCAAAAT | 1538 |
| | CGGTGA | |
| R3046_CasPhi12_S | ATTGCTCCTTACGAGGAGACGAGAATCAAAA | 1539 |
| | TCGGTG | |
| R3047_CasPhi12_S | ATTGCTCCTTACGAGGAGACACCGATTTTGA | 1540 |
| | TTCTCA | |
| R3048_CasPhi12_S | ATTGCTCCTTACGAGGAGACTTTGAGAATCA | 1541 |
| | AAATCG | |
| R3049_CasPhi12_S | ATTGCTCCTTACGAGGAGACGTTTGAGAATC | 1542 |
| | AAAATC | |
| R3050_CasPhi12_S | ATTGCTCCTTACGAGGAGACTGATTCTCAAA | 1543 |
| | CAAATG | |
| R3051_CasPhi12_S | ATTGCTCCTTACGAGGAGACGATTCTCAAAC | 1544 |
| | AAATGT | |
| R3052_CasPhi12_S | ATTGCTCCTTACGAGGAGACATTCTCAAACA | 1545 |
| | AATGTG | |
| R3053_CasPhi12_S | ATTGCTCCTTACGAGGAGACTGACACATTTG | 1546 |
| | TTTGAG | |
| R3054_CasPhi12_S | ATTGCTCCTTACGAGGAGACTCAAACAAATG | 1547 |
| | TGTCAC | |
| R3055_CasPhi12_S | ATTGCTCCTTACGAGGAGACGTGACACATTT | 1548 |
| | GTTTGA | |
| R3056_CasPhi12_S | ATTGCTCCTTACGAGGAGACCTTTGTGACAC | 1549 |
| | ATTTGT | |
| R3057_CasPhi12_S | ATTGCTCCTTACGAGGAGACTGATGTGTATA | 1550 |
| | TCACAG | |
| R3058_CasPhi12_S | ATTGCTCCTTACGAGGAGACTCTGTGATATA | 1551 |
| | CACATC | |
| R3059_CasPhi12_S | ATTGCTCCTTACGAGGAGACGTCTGTGATAT | 1552 |
| | ACACAT | |
| R3060_CasPhi12_S | ATTGCTCCTTACGAGGAGACTGTCTGTGATA | 1553 |
| | TACACA | |
| R3061_CasPhi12_S | ATTGCTCCTTACGAGGAGACAAGTCCATAGA | 1554 |
| | CCTCAT | |
| R3062_CasPhi12_S | ATTGCTCCTTACGAGGAGACCTCTTGAAGTC | 1555 |
| | CATAGA | |
| R3063_CasPhi12_S | ATTGCTCCTTACGAGGAGACAAGAGCAACAG | 1556 |
| | TGCTGT | |
| R3064_CasPhi12_S | ATTGCTCCTTACGAGGAGACCTCCAGGCCAC | 1557 |
| | AGCACT | |
| R3065_CasPhi12_S | ATTGCTCCTTACGAGGAGACTTGCTCCAGGC | 1558 |
| | CACAGC | |
| R3066_CasPhi12_S | ATTGCTCCTTACGAGGAGACGTTGCTCCAGG | 1559 |
| | CCACAG | |
| R3067_CasPhi12_S | ATTGCTCCTTACGAGGAGACCACATGCAAAG | 1560 |
| | TCAGAT | |
| R3068_CasPhi12_S | ATTGCTCCTTACGAGGAGACGCACATGCAAA | 1561 |
| | GTCAGA | |
| R3069_CasPhi12_S | ATTGCTCCTTACGAGGAGACGCATGTGCAAA | 1562 |
| | CGCCTT | |
| R3070_CasPhi12_S | ATTGCTCCTTACGAGGAGACAAGGCGTTTGC | 1563 |
| | ACATGC | |
| R3071_CasPhi12_S | ATTGCTCCTTACGAGGAGACCATGTGCAAAC | 1564 |
| | GCCTTC | |
| R3072_CasPhi12_S | ATTGCTCCTTACGAGGAGACTTGAAGGCGTT | 1565 |
| | TGCACA | |
| R3073_CasPhi12_S | ATTGCTCCTTACGAGGAGACAACAACAGCAT | 1566 |
| | TATTCC | |
| R3074_CasPhi12_S | ATTGCTCCTTACGAGGAGACTGGAATAATGC | 1567 |
| | TGTTGT | |
| R3075_CasPhi12_S | ATTGCTCCTTACGAGGAGACTTCCAGAAGAC | 1568 |
| | ACCTTC | |
| R3076_CasPhi12_S | ATTGCTCCTTACGAGGAGACCAGAAGACACC | 1569 |
| | TTCTTC | |
| R3077_CasPhi12_S | ATTGCTCCTTACGAGGAGACCCTGGGCTGGG | 1570 |
| | GAAGAA | |
| R3078_CasPhi12_S | ATTGCTCCTTACGAGGAGACTTCCCCAGCCC | 1571 |
| | AGGTAA | |
| R3079_CasPhi12_S | ATTGCTCCTTACGAGGAGACCCCAGCCCAGG | 1572 |
| | TAAGGG | |
| R3080_CasPhi12_S | ATTGCTCCTTACGAGGAGACTAAAAGGAAAA | 1573 |
| | ACAGAC | |
| R3081_CasPhi12_S | ATTGCTCCTTACGAGGAGACCTAAAAGGAAA | 1574 |
| | AACAGA | |
| R3082_CasPhi12_S | ATTGCTCCTTACGAGGAGACTTCCTTTTAGAA | 1575 |
| | AGTTC | |
| R3083_CasPhi12_S | ATTGCTCCTTACGAGGAGACTCCTTTTAGAA | 1576 |
| | AGTTCC | |
| R3084_CasPhi12_S | ATTGCTCCTTACGAGGAGACCCTTTTAGAAA | 1577 |
| | GTTCCT | |
| R3085_CasPhi12_S | ATTGCTCCTTACGAGGAGACCTTTTAGAAAG | 1578 |
| | TTCCTG | |
| R3086_CasPhi12_S | ATTGCTCCTTACGAGGAGACTAGAAAGTTCC | 1579 |
| | TGTGAT | |
| R3136_CasPhi12_S | ATTGCTCCTTACGAGGAGACAGAAAGTTCCT | 1580 |
| | GTGATG | |
| R3137_CasPhi12_S | ATTGCTCCTTACGAGGAGACGAAAGTTCCTG | 1581 |
| | TGATGT | |
| R3138_CasPhi12_S | ATTGCTCCTTACGAGGAGACACATCACAGGA | 1582 |
| | ACTTTC | |
| R3139_CasPhi12_S | ATTGCTCCTTACGAGGAGACCTGTGATGTCA | 1583 |
| | AGCTGG | |
| R3140_CasPhi12_S | ATTGCTCCTTACGAGGAGACTCGACCAGCTT | 1584 |
| | GACATC | |
| R3141_CasPhi12_S | ATTGCTCCTTACGAGGAGACCTCGACCAGCT | 1585 |
| | TGACAT | |
| R3142_CasPhi12_S | ATTGCTCCTTACGAGGAGACTCTCGACCAGC | 1586 |
| | TTGACA | |
| R3143_CasPhi12_S | ATTGCTCCTTACGAGGAGACAAAGCTTTTCT | 1587 |
| | CGACCA | |
| R3144_CasPhi12_S | ATTGCTCCTTACGAGGAGACCAAAGCTTTTC | 1588 |
| | TCGACC | |
| R3145_CasPhi12_S | ATTGCTCCTTACGAGGAGACCCTGTTTCAAA | 1589 |
| | GCTTTT | |
| R3146_CasPhi12_S | ATTGCTCCTTACGAGGAGACGAAACAGGTAA | 1590 |
| | GACAGG | |
| R3147_CasPhi12_S | ATTGCTCCTTACGAGGAGACAAACAGGTAAG | 1591 |
| | ACAGGG | |

TABLE Z
CasΦ.12 gRNAs targeting human B2M in T cells

| | Repeat + spacer RNA Sequence (5′ → 3′), | |
|---|---|---|
| Name | shown as DNA | SEQ ID NO |
| R3115_CasPhi12_S | ATTGCTCCTTACGAGGAGACCATCCATCCGA | 1592 |
| | CATTGA | |
| R3116_CasPhi12_S | ATTGCTCCTTACGAGGAGACATCCATCCGAC | 1593 |
| | ATTGAA | |
| R3117_CasPhi12_S | ATTGCTCCTTACGAGGAGACAGTAAGTCAAC | 1594 |
| | TTCAAT | |
| R3118_CasPhi12_S | ATTGCTCCTTACGAGGAGACTTCAGTAAGTC | 1595 |
| | AACTTC | |
| R3119_CasPhi12_S | ATTGCTCCTTACGAGGAGACAAGTTGACTTA | 1596 |
| | CTGAAG | |
| R3120_CasPhi12_S | ATTGCTCCTTACGAGGAGACACTTACTGAAG | 1597 |
| | AATGGA | |
| R3121_CasPhi12_S | ATTGCTCCTTACGAGGAGACTCTCTCCATTCT | 1598 |
| | TCAGT | |
| R3122_CasPhi12_S | ATTGCTCCTTACGAGGAGACCTGAAGAATGG | 1599 |
| | AGAGAG | |
| R3123_CasPhi12_S | ATTGCTCCTTACGAGGAGACAATTCTCTCTCC | 1600 |
| | ATTCT | |
| R3124_CasPhi12_S | ATTGCTCCTTACGAGGAGACCAATTCTCTCTC | 1601 |
| | CATTC | |
| R3125_CasPhi12_S | ATTGCTCCTTACGAGGAGACTCAATTCTCTCT | 1602 |
| | CCATT | |
| R3126_CasPhi12_S | ATTGCTCCTTACGAGGAGACTTCAATTCTCTC | 1603 |
| | TCCAT | |
| R3127_CasPhi12_S | ATTGCTCCTTACGAGGAGACAAAAAGTGGAG | 1604 |
| | CATTCA | |
| R3128_CasPhi12_S | ATTGCTCCTTACGAGGAGACCTGAAAGACAA | 1605 |
| | GTCTGA | |
| R3129_CasPhi12_S | ATTGCTCCTTACGAGGAGACAGACTTGTCTTT | 1606 |
| | CAGCA | |
| R3130_CasPhi12_S | ATTGCTCCTTACGAGGAGACTCTTTCAGCAA | 1607 |
| | GGACTG | |
| R3131_CasPhi12_S | ATTGCTCCTTACGAGGAGACCAGCAAGGACT | 1608 |
| | GGTCTT | |
| R3132_CasPhi12_S | ATTGCTCCTTACGAGGAGACAGCAAGGACTG | 1609 |
| | GTCTTT | |
| R3133_CasPhi12_S | ATTGCTCCTTACGAGGAGACCTATCTCTTGTA | 1610 |
| | CTACA | |
| R3134_CasPhi12_S | ATTGCTCCTTACGAGGAGACTATCTCTTGTAC | 1611 |
| | TACAC | |
| R3135_CasPhi12_S | ATTGCTCCTTACGAGGAGACAGTGTAGTACA | 1612 |
| | AGAGAT | |
| R3148_CasPhi12_S | ATTGCTCCTTACGAGGAGACTACTACACTGA | 1613 |
| | ATTCAC | |
| R3149_CasPhi12_S | ATTGCTCCTTACGAGGAGACAGTGGGGGTGA | 1614 |
| | ATTCAG | |
| R3150_CasPhi12_S | ATTGCTCCTTACGAGGAGACCAGTGGGGGTG | 1615 |
| | AATTCA | |
| R3151_CasPhi12_S | ATTGCTCCTTACGAGGAGACTCAGTGGGGGT | 1616 |
| | GAATTC | |
| R3152_CasPhi12_S | ATTGCTCCTTACGAGGAGACTTCAGTGGGGG | 1617 |
| | TGAATT | |
| R3153_CasPhi12_S | ATTGCTCCTTACGAGGAGACACCCCCACTGA | 1618 |
| | AAAAGA | |
| R3154_CasPhi12_S | ATTGCTCCTTACGAGGAGACACACGGCAGGC | 1619 |
| | ATACTC | |
| R3155_CasPhi12_S | ATTGCTCCTTACGAGGAGACGGCTGTGACAA | 1620 |
| | AGTCAC | |
| R3156_CasPhi12_S | ATTGCTCCTTACGAGGAGACGTCACAGCCCA | 1621 |
| | AGATAG | |
| R3157_CasPhi12_S | ATTGCTCCTTACGAGGAGACTCACAGCCCAA | 1622 |
| | GATAGT | |
| R3158_CasPhi12_S | ATTGCTCCTTACGAGGAGACACTATCTTGGG | 1623 |
| | CTGTGA | |
| R3159_CasPhi12_S | ATTGCTCCTTACGAGGAGACCCCCACTTAAC | 1624 |
| | TATCTT | |

TABLE AA
CasΦ.12 gRNAs targeting human PD1 in T cells

| Name | Repeat + spacer RNA Sequence (5′ → 3′) | SEQ ID NO |
|---|---|---|
| R2921_CasPhi12_S | AUUGCUCCUUACGAGGAGACCCUUCCGC | 1625 |
| | UCACCUCCG | |
| R2922_CasPhi12_S | AUUGCUCCUUACGAGGAGACCCUUCCGC | 1626 |
| | UCACCUCCG | |
| R2923_CasPhi12_S | AUUGCUCCUUACGAGGAGACCGCUCACC | 1627 |
| | UCCGCCUGA | |
| R2924_CasPhi12_S | AUUGCUCCUUACGAGGAGACUCCACUGC | 1628 |
| | UCAGGCGGA | |
| R2925_CasPhi12_S | AUUGCUCCUUACGAGGAGACUAGCACCG | 1629 |
| | CCCAGACGA | |
| R2926_CasPhi12_S | AUUGCUCCUUACGAGGAGACAGGCAUGC | 1630 |
| | AGAUCCCAC | |
| R2927_CasPhi12_S | AUUGCUCCUUACGAGGAGACCACAGGCG | 1631 |
| | CCCUGGCCA | |
| R2928_CasPhi12_S | AUUGCUCCUUACGAGGAGACUCUGGGCG | 1632 |
| | GUGCUACAA | |
| R2929_CasPhi12_S | AUUGCUCCUUACGAGGAGACGCAUGCCU | 1633 |
| | GGAGCAGCC | |
| R2930_CasPhi12_S | AUUGCUCCUUACGAGGAGACUAGCACCG | 1634 |
| | CCCAGACGA | |
| R2931_CasPhi12_S | AUUGCUCCUUACGAGGAGACUGGCCGCC | 1635 |
| | AGCCCAGUU | |
| R2932_CasPhi12_S | AUUGCUCCUUACGAGGAGACCUUCCGCU | 1636 |
| | CACCUCCGC | |
| R2933_CasPhi12_S | AUUGCUCCUUACGAGGAGACCAGGGCCU | 1637 |
| | GUCUGGGGA | |
| R2934_CasPhi12_S | AUUGCUCCUUACGAGGAGACUCCCCAGC | 1638 |
| | CCUGCUCGU | |
| R2935_CasPhi12_S | AUUGCUCCUUACGAGGAGACGGUCACCA | 1639 |
| | CGAGCAGGG | |
| R2936_CasPhi12_S | AUUGCUCCUUACGAGGAGACUCCCCUUC | 1640 |
| | GGUCACCAC | |
| R2937_CasPhi12_S | AUUGCUCCUUACGAGGAGACGAGAAGCU | 1641 |
| | GCAGGUGAA | |
| R2938_CasPhi12_S | AUUGCUCCUUACGAGGAGACACCUGCAG | 1642 |
| | CUUCUCCAA | |
| R2939_CasPhi12_S | AUUGCUCCUUACGAGGAGACUCCAACAC | 1643 |
| | AUCGGAGAG | |
| R2940_CasPhi12_S | AUUGCUCCUUACGAGGAGACGCACGAAG | 1644 |
| | CUCUCCGAU | |
| R2941_CasPhi12_S | AUUGCUCCUUACGAGGAGACAGCACGAA | 1645 |
| | GCUCUCCGA | |
| R2942_CasPhi12_S | AUUGCUCCUUACGAGGAGACGUGCUAAA | 1646 |
| | CUGGUACCG | |
| R2943_CasPhi12_S | AUUGCUCCUUACGAGGAGACCUGGGGCU | 1647 |
| | CAUGCGGUA | |
| R2944_CasPhi12_S | AUUGCUCCUUACGAGGAGACUCCGUCUG | 1648 |
| | GUUGCUGGG | |
| R2945_CasPhi12_S | AUUGCUCCUUACGAGGAGACCCCGAGGA | 1649 |
| | CCGCAGCCA | |
| R2946_CasPhi12_S | AUUGCUCCUUACGAGGAGACUGUGACAC | 1650 |
| | GGAAGCGGC | |
| R2947_CasPhi12_S | AUUGCUCCUUACGAGGAGACCGUGUCAC | 1651 |
| | ACAACUGCC | |
| R2948_CasPhi12_S | AUUGCUCCUUACGAGGAGACGGCAGUUG | 1652 |
| | UGUGACACG | |
| R2949_CasPhi12_S | AUUGCUCCUUACGAGGAGACCACAUGAG | 1653 |
| | CGUGGUCAG | |
| R2950_CasPhi12_S | AUUGCUCCUUACGAGGAGACCGCCGGGC | 1654 |
| | CCUGACCAC | |
| R2951_CasPhi12_S | AUUGCUCCUUACGAGGAGACGGGGCCAG | 1655 |
| | GGAGAUGGC | |
| R2952_CasPhi12_S | AUUGCUCCUUACGAGGAGACAUCUGCGC | 1656 |
| | CUUGGGGGC | |
| R2953_CasPhi12_S | AUUGCUCCUUACGAGGAGACGAUCUGCG | 1657 |
| | CCUUGGGGG | |
| R2954_CasPhi12_S | AUUGCUCCUUACGAGGAGACCCAGACAG | 1658 |
| | GCCCUGGAA | |
| R2955_CasPhi12_S | AUUGCUCCUUACGAGGAGACCCAGCCCU | 1659 |
| | GCUCGUGGU | |
| R2956_CasPhi12_S | AUUGCUCCUUACGAGGAGACUCUCUGGA | 1660 |
| | AGGGCACAA | |
| R2957_CasPhi12_S | AUUGCUCCUUACGAGGAGACGUGCCCUU | 1661 |
| | CCAGAGAGA | |
| R2958_CasPhi12_S | AUUGCUCCUUACGAGGAGACUGCCCUUC | 1662 |
| | CAGAGAGAA | |
| R2959_CasPhi12_S | AUUGCUCCUUACGAGGAGACUGCCCUUC | 1663 |
| | UCUCUGGAA | |
| R2960_CasPhi12_S | AUUGCUCCUUACGAGGAGACCAGAGAGA | 1664 |
| | AGGGCAGAA | |
| R2961_CasPhi12_S | AUUGCUCCUUACGAGGAGACGAACUGGC | 1665 |
| | CGGCUGGCC | |
| R2962_CasPhi12_S | AUUGCUCCUUACGAGGAGACGGAACUGG | 1666 |
| | CCGGCUGGC | |
| R2963_CasPhi12_S | AUUGCUCCUUACGAGGAGACCAAACCCU | 1667 |
| | GGUGGUUGG | |
| R2964_CasPhi12_S | AUUGCUCCUUACGAGGAGACGUGUCGUG | 1668 |
| | GGCGGCCUG | |
| R2965_CasPhi12_S | AUUGCUCCUUACGAGGAGACCCUCGUGC | 1669 |
| | GGCCCGGGA | |
| R2966_CasPhi12_S | AUUGCUCCUUACGAGGAGACUCCCUGCA | 1670 |
| | GAGAAACAC | |
| R2967_CasPhi12_S | AUUGCUCCUUACGAGGAGACCUCUGCAG | 1671 |
| | GGACAAUAG | |
| R2968_CasPhi12_S | AUUGCUCCUUACGAGGAGACUCUGCAGG | 1672 |
| | GACAAUAGG | |
| R2969_CasPhi12_S | AUUGCUCCUUACGAGGAGACCUCCUCAA | 1673 |
| | AGAAGGAGG | |
| R2970_CasPhi12_S | AUUGCUCCUUACGAGGAGACUCCUCAAA | 1674 |
| | GAAGGAGGA | |
| R2971_CasPhi12_S | AUUGCUCCUUACGAGGAGACUCUGUGGA | 1675 |
| | CUAUGGGGA | |
| R2972_CasPhi12_S | AUUGCUCCUUACGAGGAGACUCUCGCCA | 1676 |
| | CUGGAAAUC | |
| R2973_CasPhi12_S | AUUGCUCCUUACGAGGAGACCCAGUGGC | 1677 |
| | GAGAGAAGA | |
| R2974_CasPhi12_S | AUUGCUCCUUACGAGGAGACCAGUGGCG | 1678 |
| | AGAGAAGAC | |
| R2975_CasPhi12_S | AUUGCUCCUUACGAGGAGACCGCUAGGA | 1679 |
| | AAGACAAUG | |
| R2976_CasPhi12_S | AUUGCUCCUUACGAGGAGACUCUUUCCU | 1680 |
| | AGCGGAAUG | |
| R2977_CasPhi12_S | AUUGCUCCUUACGAGGAGACCCUAGCGG | 1681 |
| | AAUGGGCAC | |
| R2978_CasPhi12_S | AUUGCUCCUUACGAGGAGACCUAGCGGA | 1682 |
| | AUGGGCACC | |
| R2979_CasPhi12_S | AUUGCUCCUUACGAGGAGACGCCCCUCU | 1683 |
| | GACCGGCUU | |
| R2980_CasPhi12_S | AUUGCUCCUUACGAGGAGACCUUGGCCA | 1684 |
| | CCAGUGUUC | |
| R2981_CasPhi12_S | AUUGCUCCUUACGAGGAGACGCCACCAG | 1685 |
| | UGUUCUGCA | |
| R2982_CasPhi12_S | AUUGCUCCUUACGAGGAGACUGCAGACC | 1686 |
| | CUCCACCAU | |
| R2983_CasPhi12_S | AUUGCUCCUUACGAGGAGACUCCUGAGG | 1687 |
| | AAAUGCGCU | |
| R2984_CasPhi12_S | AUUGCUCCUUACGAGGAGACCCUCAGGA | 1688 |
| | GAAGCAGGC | |
| R2985_CasPhi12_S | AUUGCUCCUUACGAGGAGACCUCAGGAG | 1689 |
| | AAGCAGGCA | |
| R2986_CasPhi12_S | AUUGCUCCUUACGAGGAGACCAGGCCGU | 1690 |
| | CCAGGGGCU | |
| R2987_CasPhi12_S | AUUGCUCCUUACGAGGAGACAGACAUGA | 1691 |
| | GUCCUGUGG | |
| R2988_CasPhi12_S | AUUGCUCCUUACGAGGAGACAGGUCCUG | 1692 |
| | CCAGCACAG | |
| R2989_CasPhi12_S | AUUGCUCCUUACGAGGAGACAGGGAGCU | 1693 |
| | GGACGCAGG | |
| R2990_CasPhi12_S | AUUGCUCCUUACGAGGAGACAGCCCCGG | 1694 |
| | GCCGCAGGC | |
| R2991_CasPhi12_S | AUUGCUCCUUACGAGGAGACAGGCAGGA | 1695 |
| | GGCUCCGGG | |
| R2992_CasPhi12_S | AUUGCUCCUUACGAGGAGACGGGGCUGG | 1696 |
| | UUGGAGAUG | |
| R2993_CasPhi12_S | AUUGCUCCUUACGAGGAGACGAGAUGGC | 1697 |
| | CUUGGAGCA | |
| R2994_CasPhi12_S | AUUGCUCCUUACGAGGAGACGCUGCUCC | 1698 |
| | AAGGCCAUC | |
| R2995_CasPhi12_S | AUUGCUCCUUACGAGGAGACGAGCAGCC | 1699 |
| | AAGGUGCCC | |
| R2996_CasPhi12_S | AUUGCUCCUUACGAGGAGACGGGAUGCC | 1700 |
| | ACUGCCAGG | |
| R2997_CasPhi12_S | AUUGCUCCUUACGAGGAGACCGGGAUGC | 1701 |
| | CACUGCCAG | |
| R2998_CasPhi12_S | AUUGCUCCUUACGAGGAGACGGCCCUGC | 1702 |
| | GUCCAGGGC | |
| R2999_CasPhi12_S | AUUGCUCCUUACGAGGAGACUCUGCUCC | 1703 |
| | CUGCAGGCC | |
| R3000_CasPhi12_S | AUUGCUCCUUACGAGGAGACUCUAGGCC | 1704 |
| | UGCAGGGAG | |
| R3001_CasPhi12_S | AUUGCUCCUUACGAGGAGACCCUGAAAC | 1705 |
| | UUCUCUAGG | |
| R3002_CasPhi12_S | AUUGCUCCUUACGAGGAGACUGACCUUC | 1706 |
| | CCUGAAACU | |
| R3003_CasPhi12_S | AUUGCUCCUUACGAGGAGACCAGGGAAG | 1707 |
| | GUCAGAAGA | |
| R3004_CasPhi12_S | AUUGCUCCUUACGAGGAGACAGGGAAGG | 1708 |
| | UCAGAAGAG | |
| R3005_CasPhi12_S | AUUGCUCCUUACGAGGAGACCUGCCCUG | 1709 |
| | CCCACCACA | |
| R3006_CasPhi12_S | AUUGCUCCUUACGAGGAGACCCUGCCCU | 1710 |
| | GCCCACCAC | |
| R3007_CasPhi12_S | AUUGCUCCUUACGAGGAGACACACAUGC | 1711 |
| | CCAGGCAGC | |
| R3008_CasPhi12_S | AUUGCUCCUUACGAGGAGACCACAUGCC | 1712 |
| | CAGGCAGCA | |
| R3009_CasPhi12_S | AUUGCUCCUUACGAGGAGACCCUGCCCC | 1713 |
| | ACAAAGGGC | |
| R3010_CasPhi12_S | AUUGCUCCUUACGAGGAGACGUGGGGCA | 1714 |
| | GGGAAGCUG | |
| R3011_CasPhi12_S | AUUGCUCCUUACGAGGAGACUGGGGCAG | 1715 |
| | GGAAGCUGA | |
| R3012_CasPhi12_S | AUUGCUCCUUACGAGGAGACCUGCCUCA | 1716 |
| | GCUUCCCUG | |
| R3013_CasPhi12_S | AUUGCUCCUUACGAGGAGACCAGGCCCA | 1717 |
| | GCCAGCACU | |
| R3014_CasPhi12_S | AUUGCUCCUUACGAGGAGACAGGCCCAG | 1718 |
| | CCAGCACUC | |
| R3015_CasPhi12_S | AUUGCUCCUUACGAGGAGACCACCCCAG | 1719 |
| | CCCCUCACA | |
| R3016_CasPhi12_S | AUUGCUCCUUACGAGGAGACGGACCGUA | 1720 |
| | GGAUGUCCC | |

TABLE AB
shortened CasΦ.12 gRNAs targeting human CIITA

| Name | Repeat + spacer RNA Sequence (5′ → 3′) | SEQ ID NO |
|---|---|---|
| R4503_CasPhi12_ | AUUGCUCCUUACGAGGAGACCUACACAA | 1721 |
| C2TA_T1.1_S | UGCGUUGCC | |
| R4504_CasPhi12_ | AUUGCUCCUUACGAGGAGACGGGCUCUG | 1722 |
| C2TA_T1.2_S | ACAGGUAGG | |
| R4505_CasPhi12_ | AUUGCUCCUUACGAGGAGACUGUAGGAA | 1723 |
| C2TA_T1.3_S | UCCCAGCCA | |
| R4506_CasPhi12_ | AUUGCUCCUUACGAGGAGACCCUGGCUC | 1724 |
| C2TA_T1.8_S | CACGCCCUG | |
| R4507_CasPhi12_ | AUUGCUCCUUACGAGGAGACGGGAAGCU | 1725 |
| C2TA_T1.9_S | GAGGGCACG | |
| R4508_CasPhi12_ | AUUGCUCCUUACGAGGAGACACAGCGAU | 1726 |
| C2TA_T2.1_S | GCUGACCCC | |
| R4509_CasPhi12_ | AUUGCUCCUUACGAGGAGACUUAACAGC | 1727 |
| C2TA_T2.2_S | GAUGCUGAC | |
| R4510_CasPhi12_ | AUUGCUCCUUACGAGGAGACUAUGACCA | 1728 |
| C2TA_T2.3_S | GAUGGACCU | |
| R4511_CasPhi12_ | AUUGCUCCUUACGAGGAGACGGGCCCCU | 1729 |
| C2TA_T2.4_S | AGAAGGUGG | |
| R4512_CasPhi12_ | AUUGCUCCUUACGAGGAGACUAGGGGCC | 1730 |
| C2TA_T2.5_S | CCAACUCCA | |
| R4513_CasPhi12_ | AUUGCUCCUUACGAGGAGACAGAAGCUC | 1731 |
| C2TA_T2.6_S | CAGGUAGCC | |
| R4514_CasPhi12_ | AUUGCUCCUUACGAGGAGACUCCAGCCA | 1732 |
| C2TA_T2.7_S | GGUCCAUCU | |
| R4515_CasPhi12_ | AUUGCUCCUUACGAGGAGACUUCUCCAG | 1733 |
| C2TA_T2.8_S | CCAGGUCCA | |
| R5200_CasPhi12_S | AUUGCUCCUUACGAGGAGACAGCAGGCU | 2290 |
| | GUUGUGUGA | |
| R5201_CasPhi12_S | AUUGCUCCUUACGAGGAGACCAUGUCAC | 2291 |
| | ACAACAGCC | |
| R5202_CasPhi12_S | AUUGCUCCUUACGAGGAGACUGUGACAU | 2292 |
| | GGAAGGUGA | |
| R5203_CasPhi12_S | AUUGCUCCUUACGAGGAGACAUCACCUU | 2293 |
| | CCAUGUCAC | |
| R5204_CasPhi12_S | AUUGCUCCUUACGAGGAGACGCAUAAGC | 2294 |
| | CUCCCUGGU | |
| R5205_CasPhi12_S | AUUGCUCCUUACGAGGAGACCAGGACUC | 2295 |
| | CCAGCUGGA | |
| R5206_CasPhi12_S | AUUGCUCCUUACGAGGAGACCUCAGGCC | 2296 |
| | CUCCAGCUG | |
| R5207_CasPhi12_S | AUUGCUCCUUACGAGGAGACUGCUGGCA | 2297 |
| | UCUCCAUAC | |
| R5208_CasPhi12_S | AUUGCUCCUUACGAGGAGACUGCCCAAC | 2298 |
| | UUCUGCUGG | |
| R5209_CasPhi12_S | AUUGCUCCUUACGAGGAGACCUGCCCAA | 2299 |
| | CUUCUGCUG | |
| R5210_CasPhi12_S | AUUGCUCCUUACGAGGAGACUCUGCCCA | 2300 |
| | ACUUCUGCU | |
| R5211_CasPhi12_S | AUUGCUCCUUACGAGGAGACUGACUUUU | 2301 |
| | CUGCCCAAC | |
| R5212_CasPhi12_S | AUUGCUCCUUACGAGGAGACCUGACUUU | 2302 |
| | UCUGCCCAA | |
| R5213_CasPhi12_S | AUUGCUCCUUACGAGGAGACUCUGACUU | 2303 |
| | UUCUGCCCA | |
| R5214_CasPhi12_S | AUUGCUCCUUACGAGGAGACCCAGAGGA | 2304 |
| | GCUUCCGGC | |
| R5215_CasPhi12_S | AUUGCUCCUUACGAGGAGACAGGUCUGC | 2305 |
| | CGGAAGCUC | |
| R5216_CasPhi12_S | AUUGCUCCUUACGAGGAGACCGGCAGAC | 2306 |
| | CUGAAGCAC | |
| R5217_CasPhi12_S | AUUGCUCCUUACGAGGAGACCAGUGCUU | 2307 |
| | CAGGUCUGC | |
| R5218_CasPhi12_S | AUUGCUCCUUACGAGGAGACAACAGCGC | 2308 |
| | AGGCAGUGG | |
| R5219_CasPhi12_S | AUUGCUCCUUACGAGGAGACAACCAGGA | 2309 |
| | GCCAGCCUC | |
| R5220_CasPhi12_S | AUUGCUCCUUACGAGGAGACUCCAGGCG | 2310 |
| | CAUCUGGCC | |
| R5221_CasPhi12_S | AUUGCUCCUUACGAGGAGACCUCCAGGC | 2311 |
| | GCAUCUGGC | |
| R5222_CasPhi12_S | AUUGCUCCUUACGAGGAGACUCUCCAGG | 2312 |
| | CGCAUCUGG | |
| R5223_CasPhi12_S | AUUGCUCCUUACGAGGAGACCUCCAGUU | 2313 |
| | CCUCGUUGA | |
| R5224_CasPhi12_S | AUUGCUCCUUACGAGGAGACUCCAGUUC | 2314 |
| | CUCGUUGAG | |
| R5225_CasPhi12_S | AUUGCUCCUUACGAGGAGACAGGCAGCU | 2315 |
| | CAACGAGGA | |
| R5226_CasPhi12_S | AUUGCUCCUUACGAGGAGACCUCGUUGA | 2316 |
| | GCUGCCUGA | |
| R5227_CasPhi12_S | AUUGCUCCUUACGAGGAGACAGCUGCCU | 2317 |
| | GAAUCUCCC | |
| R5228_CasPhi12_S | AUUGCUCCUUACGAGGAGACGUCCCCAC | 2318 |
| | CAUCUCCAC | |
| R5229_CasPhi12_S | AUUGCUCCUUACGAGGAGACUCCCCACC | 2319 |
| | AUCUCCACU | |
| R5230_CasPhi12_S | AUUGCUCCUUACGAGGAGACCCAGAGCC | 2320 |
| | CAUGGGGCA | |
| R5231_CasPhi12_S | AUUGCUCCUUACGAGGAGACGCCAGAGC | 2321 |
| | CCAUGGGGC | |
| R5232_CasPhi12_S | AUUGCUCCUUACGAGGAGACCAGCCUCA | 2322 |
| | GAGAUUUGC | |
| R5233_CasPhi12_S | AUUGCUCCUUACGAGGAGACGGAGGCCG | 2323 |
| | UGGACAGUG | |
| R5234_CasPhi12_S | AUUGCUCCUUACGAGGAGACACUGUCCA | 2324 |
| | CGGCCUCCC | |
| R5235_CasPhi12_S | AUUGCUCCUUACGAGGAGACGCUCCAUC | 2325 |
| | AGCCACUGA | |
| R5236_CasPhi12_S | AUUGCUCCUUACGAGGAGACAGGCAUGC | 2326 |
| | UGGGCAGGU | |
| R5237_CasPhi12_S | AUUGCUCCUUACGAGGAGACCUCGGGAG | 2327 |
| | GUCAGGGCA | |
| R5238_CasPhi12_S | AUUGCUCCUUACGAGGAGACGCUCGGGA | 2328 |
| | GGUCAGGGC | |
| R5239_CasPhi12_S | AUUGCUCCUUACGAGGAGACGAGACCUC | 2329 |
| | UCCAGCUGC | |
| R5240_CasPhi12_S | AUUGCUCCUUACGAGGAGACUUGGAGAC | 2330 |
| | CUCUCCAGC | |
| R5241_CasPhi12_S | AUUGCUCCUUACGAGGAGACGAAGCUUG | 2331 |
| | UUGGAGACC | |
| R5242_CasPhi12_S | AUUGCUCCUUACGAGGAGACGGAAGCUU | 2332 |
| | GUUGGAGAC | |
| R5243_CasPhi12_S | AUUGCUCCUUACGAGGAGACUGGAAGCU | 2333 |
| | UGUUGGAGA | |
| R5244_CasPhi12_S | AUUGCUCCUUACGAGGAGACUACCGCUC | 2334 |
| | ACUGCAGGA | |
| R5245_CasPhi12_S | AUUGCUCCUUACGAGGAGACCUGCUGCU | 2335 |
| | CCUCUCCAG | |
| R5246_CasPhi12_S | AUUGCUCCUUACGAGGAGACCCGCUCCA | 2336 |
| | GGCUCUUGC | |
| R5247_CasPhi12_S | AUUGCUCCUUACGAGGAGACUGCCCAGU | 2337 |
| | CCGGGGUGG | |
| R5248_CasPhi12_S | AUUGCUCCUUACGAGGAGACGGCCAGCU | 2338 |
| | GCCGUUCUG | |
| R5249_CasPhi12_S | AUUGCUCCUUACGAGGAGACGCAGCCAA | 2339 |
| | CAGCACCUC | |
| R5250_CasPhi12_S | AUUGCUCCUUACGAGGAGACGCUGCCAA | 2340 |
| | GGAGCACCG | |
| R5251_CasPhi12_S | AUUGCUCCUUACGAGGAGACCCCAGCAC | 2341 |
| | AGCAAUCAC | |
| R5252_CasPhi12_S | AUUGCUCCUUACGAGGAGACGCCCAGCA | 2342 |
| | CAGCAAUCA | |
| R5253_CasPhi12_S | AUUGCUCCUUACGAGGAGACCUGUGCUG | 2343 |
| | GGCAAAGCU | |
| R5254_CasPhi12_S | AUUGCUCCUUACGAGGAGACCCCUGACC | 2344 |
| | AGCUUUGCC | |
| R5255_CasPhi12_S | AUUGCUCCUUACGAGGAGACGGCUGGGG | 2345 |
| | CAGUGAGCC | |
| R5256_CasPhi12_S | AUUGCUCCUUACGAGGAGACUGGCCGGC | 2346 |
| | UUCCCCAGU | |
| R5257_CasPhi12_S | AUUGCUCCUUACGAGGAGACCCCAGUAC | 2347 |
| | GACUUUGUC | |
| R5258_CasPhi12_S | AUUGCUCCUUACGAGGAGACGUCUUCUC | 2348 |
| | UGUCCCCUG | |
| R5259_CasPhi12_S | AUUGCUCCUUACGAGGAGACUCUUCUCU | 2349 |
| | GUCCCCUGC | |
| R5260_CasPhi12_S | AUUGCUCCUUACGAGGAGACUCUGUCCC | 2350 |
| | CUGCCAUUG | |
| R5261_CasPhi12_S | AUUGCUCCUUACGAGGAGACAAGCAAUG | 2351 |
| | GCAGGGGAC | |
| R5262_CasPhi12_S | AUUGCUCCUUACGAGGAGACCUUGAACC | 2352 |
| | GUCCGGGGG | |
| R5263_CasPhi12_S | AUUGCUCCUUACGAGGAGACAACCGUCC | 2353 |
| | GGGGGAUGC | |
| R5264_CasPhi12_S | AUUGCUCCUUACGAGGAGACUCCCUGGG | 2354 |
| | CCCACAGCC | |
| R5265_CasPhi12_S | AUUGCUCCUUACGAGGAGACAAGAUGUG | 2355 |
| | GCUGAAAAC | |
| R5266_CasPhi12_S | AUUGCUCCUUACGAGGAGACUCAGCCAC | 2356 |
| | AUCUUGAAG | |
| R5267_CasPhi12_S | AUUGCUCCUUACGAGGAGACCAGCCACA | 2357 |
| | UCUUGAAGA | |
| R5268_CasPhi12_S | AUUGCUCCUUACGAGGAGACAGCCACAU | 2358 |
| | CUUGAAGAG | |
| R5269_CasPhi12_S | AUUGCUCCUUACGAGGAGACAAGAGACC | 2359 |
| | UGACCGCGU | |
| R5270_CasPhi12_S | AUUGCUCCUUACGAGGAGACUGCUCAUC | 2360 |
| | CUAGACGGC | |
| R5271_CasPhi12_S | AUUGCUCCUUACGAGGAGACCAGCUCCU | 2361 |
| | CGAAGCCGU | |
| R5272_CasPhi12_S | AUUGCUCCUUACGAGGAGACCGCUUCCA | 2362 |
| | GCUCCUCGA | |
| R5273_CasPhi12_S | AUUGCUCCUUACGAGGAGACGAGGAGCU | 2363 |
| | GGAAGCGCA | |
| R5274_CasPhi12_S | AUUGCUCCUUACGAGGAGACCUGCACAG | 2364 |
| | CACGUGCGG | |
| R5275_CasPhi12_S | AUUGCUCCUUACGAGGAGACUGGAAAAG | 2365 |
| | GCCGGCCAG | |
| R5276_CasPhi12_S | AUUGCUCCUUACGAGGAGACUUCUGGAA | 2366 |
| | AAGGCCGGC | |
| R5277_CasPhi12_S | AUUGCUCCUUACGAGGAGACUCCAGAAG | 2367 |
| | AAGCUGCUC | |
| R5278_CasPhi12_S | AUUGCUCCUUACGAGGAGACCCAGAAGA | 2368 |
| | AGCUGCUCC | |
| R5279_CasPhi12_S | AUUGCUCCUUACGAGGAGACCAGAAGAA | 2369 |
| | GCUGCUCCG | |
| R5280_CasPhi12_S | AUUGCUCCUUACGAGGAGACCACCCUCC | 2370 |
| | UCCUCACAG | |
| R5281_CasPhi12_S | AUUGCUCCUUACGAGGAGACCUCAGGCU | 2371 |
| | CUGGACCAG | |
| R5282_CasPhi12_S | AUUGCUCCUUACGAGGAGACGAGCUGUC | 2372 |
| | CGGCUUCUC | |
| R5283_CasPhi12_S | AUUGCUCCUUACGAGGAGACAGCUGUCC | 2373 |
| | GGCUUCUCC | |
| R5284_CasPhi12_S | AUUGCUCCUUACGAGGAGACUCCAUGGA | 2374 |
| | GCAGGCCCA | |
| R5285_CasPhi12_S | AUUGCUCCUUACGAGGAGACGAGAGCUC | 2375 |
| | AGGGAUGAC | |
| R5286_CasPhi12_S | AUUGCUCCUUACGAGGAGACAGAGCUCA | 2376 |
| | GGGAUGACA | |
| R5287_CasPhi12_S | AUUGCUCCUUACGAGGAGACGUGCUCUG | 2377 |
| | UCAUCCCUG | |
| R5288_CasPhi12_S | AUUGCUCCUUACGAGGAGACUUCUCAGU | 2378 |
| | CACAGCCAC | |
| R5289_CasPhi12_S | AUUGCUCCUUACGAGGAGACUCAGUCAC | 2379 |
| | AGCCACAGC | |
| R5290_CasPhi12_S | AUUGCUCCUUACGAGGAGACGUGCCGGG | 2380 |
| | CAGUGUGCC | |
| R5291_CasPhi12_S | AUUGCUCCUUACGAGGAGACUGCCGGGC | 2381 |
| | AGUGUGCCA | |
| R5292_CasPhi12_S | AUUGCUCCUUACGAGGAGACGCGUCCUC | 2382 |
| | CCCAAGCUC | |
| R5293_CasPhi12_S | AUUGCUCCUUACGAGGAGACGGGAGGAC | 2383 |
| | GCCAAGCUG | |
| R5294_CasPhi12_S | AUUGCUCCUUACGAGGAGACGCCAGCUC | 2384 |
| | UGCCAGGGC | |
| R5295_CasPhi12_S | AUUGCUCCUUACGAGGAGACAUGUCUGC | 2385 |
| | GGCCCAGCU | |
| R5392_CasPhi12_S | AUUGCUCCUUACGAGGAGACGAUGUCUG | 2386 |
| | CGGCCCAGC | |
| R5393_CasPhi12_S | AUUGCUCCUUACGAGGAGACCCAUCCGC | 2387 |
| | AGACGUGAG | |
| R5394_CasPhi12_S | AUUGCUCCUUACGAGGAGACGCCAUCGC | 2388 |
| | CCAGGUCCU | |
| R5395_CasPhi12_S | AUUGCUCCUUACGAGGAGACGGCCAUCG | 2389 |
| | CCCAGGUCC | |
| R5396_CasPhi12_S | AUUGCUCCUUACGAGGAGACGACUAAGC | 2390 |
| | CUUUGGCCA | |
| R5397_CasPhi12_S | AUUGCUCCUUACGAGGAGACGUCCAACA | 2391 |
| | CCCACCGCG | |
| R5398_CasPhi12_S | AUUGCUCCUUACGAGGAGACCAGGAGGA | 2392 |
| | AGCUGGGGA | |
| R5399_CasPhi12_S | AUUGCUCCUUACGAGGAGACCCCAGCUU | 2393 |
| | CCUCCUGCA | |
| R5400_CasPhi12_S | AUUGCUCCUUACGAGGAGACCUCCUGCA | 2394 |
| | AUGCUUCCU | |
| R5401_CasPhi12_S | AUUGCUCCUUACGAGGAGACCUGGGGGC | 2395 |
| | CCUGUGGCU | |
| R5402_CasPhi12_S | AUUGCUCCUUACGAGGAGACGCCACUCA | 2396 |
| | GAGCCAGCC | |
| R5403_CasPhi12_S | AUUGCUCCUUACGAGGAGACCGCCACUC | 2397 |
| | AGAGCCAGC | |
| R5404_CasPhi12_S | AUUGCUCCUUACGAGGAGACAUUUCGCC | 2398 |
| | ACUCAGAGC | |
| R5405_CasPhi12_S | AUUGCUCCUUACGAGGAGACUCCUUGAU | 2399 |
| | UUCGCCACU | |
| R5406_CasPhi12_S | AUUGCUCCUUACGAGGAGACGGGUCAAU | 2400 |
| | GCUAGGUAC | |
| R5407_CasPhi12_S | AUUGCUCCUUACGAGGAGACCUUGGGGU | 2401 |
| | CAAUGCUAG | |
| R5408_CasPhi12_S | AUUGCUCCUUACGAGGAGACUUCCUUGG | 2402 |
| | GGUCAAUGC | |
| R5409_CasPhi12_S | AUUGCUCCUUACGAGGAGACACCCCAAG | 2403 |
| | GAAGAAGAG | |
| R5410_CasPhi12_S | AUUGCUCCUUACGAGGAGACUCAUAGGG | 2404 |
| | CCUCUUCUU | |
| R5411_CasPhi12_S | AUUGCUCCUUACGAGGAGACCUGGCUGG | 2405 |
| | GCUGAUCUU | |
| R5412_CasPhi12_S | AUUGCUCCUUACGAGGAGACUGGCUGGG | 2406 |
| | CUGAUCUUC | |
| R5413_CasPhi12_S | AUUGCUCCUUACGAGGAGACCAGCCUCC | 2407 |
| | CGCCCGCUG | |
| R5414_CasPhi12_S | AUUGCUCCUUACGAGGAGACCUGUCCAC | 2408 |
| | CGAGGCAGC | |
| R5415_CasPhi12_S | AUUGCUCCUUACGAGGAGACUGCUUCCU | 2409 |
| | GUCCACCGA | |
| R5416_CasPhi12_S | AUUGCUCCUUACGAGGAGACAGGUACCU | 2410 |
| | CGCAAGCAC | |
| R5417_CasPhi12_S | AUUGCUCCUUACGAGGAGACCGAGGUAC | 2411 |
| | CUGAAGCGG | |
| R5418_CasPhi12_S | AUUGCUCCUUACGAGGAGACCAGCCUCC | 2412 |
| | UCGGCCUCG | |
| R5419_CasPhi12_S | AUUGCUCCUUACGAGGAGACGGCAGCAC | 2413 |
| | GUGGUACAG | |
| R5420_CasPhi12_S | AUUGCUCCUUACGAGGAGACGCAGCACG | 2414 |
| | UGGUACAGG | |
| R5421_CasPhi12_S | AUUGCUCCUUACGAGGAGACUCUGGGCA | 2415 |
| | CCCGCCUCA | |
| R5422_CasPhi12_S | AUUGCUCCUUACGAGGAGACCUGGGCAC | 2416 |
| | CCGCCUCAC | |
| R5423_CasPhi12_S | AUUGCUCCUUACGAGGAGACUGGGCACC | 2417 |
| | CGCCUCACG | |
| R5424_CasPhi12_S | AUUGCUCCUUACGAGGAGACCCCAGUAC | 2418 |
| | AUGUGCAUC | |
| R5425_CasPhi12_S | AUUGCUCCUUACGAGGAGACGCCCGCCG | 2419 |
| | CCUCCAAGG | |
| R5426_CasPhi12_S | AUUGCUCCUUACGAGGAGACGAGGCGGC | 2420 |
| | GGGCCAAGA | |
| R5427_CasPhi12_S | AUUGCUCCUUACGAGGAGACUCCCUGGA | 2421 |
| | CCUCCGCAG | |
| R5428_CasPhi12_S | AUUGCUCCUUACGAGGAGACGCCCCUCU | 2422 |
| | GGAUUGGGG | |
| R5429_CasPhi12_S | AUUGCUCCUUACGAGGAGACCCCCUCUG | 2423 |
| | GAUUGGGGA | |
| R5430_CasPhi12_S | AUUGCUCCUUACGAGGAGACGGGAGCCU | 2424 |
| | CGUGGGACU | |
| R5431_CasPhi12_S | AUUGCUCCUUACGAGGAGACGUCUCCCC | 2425 |
| | AUGCUGCUG | |
| R5432_CasPhi12_S | AUUGCUCCUUACGAGGAGACUCCUCUGC | 2426 |
| | UGCCUGAAG | |
| R5433_CasPhi12_S | AUUGCUCCUUACGAGGAGACAGGCAGCA | 2427 |
| | GAGGAGAAG | |
| R5434_CasPhi12_S | AUUGCUCCUUACGAGGAGACAAAGGCUC | 2428 |
| | GAUGGUGAA | |
| R5435_CasPhi12_S | AUUGCUCCUUACGAGGAGACGAAAGGCU | 2429 |
| | CGAUGGUGA | |
| R5436_CasPhi12_S | AUUGCUCCUUACGAGGAGACACCAUCGA | 2430 |
| | GCCUUUCAA | |
| R5437_CasPhi12_S | AUUGCUCCUUACGAGGAGACGCUUUGAA | 2431 |
| | AGGCUCGAU | |
| R5438_CasPhi12_S | AUUGCUCCUUACGAGGAGACAGGGACUU | 2432 |
| | GGCUUUGAA | |
| R5439_CasPhi12_S | AUUGCUCCUUACGAGGAGACCAAAGCCA | 2433 |
| | AGUCCCUGA | |
| R5440_CasPhi12_S | AUUGCUCCUUACGAGGAGACAAAGCCAA | 2434 |
| | GUCCCUGAA | |
| R5441_CasPhi12_S | AUUGCUCCUUACGAGGAGACCACAUCCU | 2435 |
| | UCAGGGACU | |
| R5442_CasPhi12_S | AUUGCUCCUUACGAGGAGACCCAGGUCU | 2436 |
| | UCCACAUCC | |
| R5443_CasPhi12_S | AUUGCUCCUUACGAGGAGACCCCAGGUC | 2437 |
| | UUCCACAUC | |
| R5444_CasPhi12_S | AUUGCUCCUUACGAGGAGACCUCGGAAG | 2438 |
| | ACACAGCUG | |
| R5445_CasPhi12_S | AUUGCUCCUUACGAGGAGACGGUCCCGA | 2439 |
| | ACAGCAGGG | |
| R5446_CasPhi12_S | AUUGCUCCUUACGAGGAGACAGGUCCCG | 2440 |
| | AACAGCAGG | |
| R5447_CasPhi12_S | AUUGCUCCUUACGAGGAGACUUUAGGUC | 2441 |
| | CCGAACAGC | |
| R5448_CasPhi12_S | AUUGCUCCUUACGAGGAGACCUUUAGGU | 2442 |
| | CCCGAACAG | |
| R5449_CasPhi12_S | AUUGCUCCUUACGAGGAGACGGGACCUA | 2443 |
| | AAGAAACUG | |
| R5450_CasPhi12_S | AUUGCUCCUUACGAGGAGACGGGAAAGC | 2444 |
| | CUGGGGGCC | |
| R5451_CasPhi12_S | AUUGCUCCUUACGAGGAGACGGGGAAAG | 2445 |
| | CCUGGGGGC | |
| R5452_CasPhi12_S | AUUGCUCCUUACGAGGAGACCCCCAAAC | 2446 |
| | UGGUGCGGA | |
| R5453_CasPhi12_S | AUUGCUCCUUACGAGGAGACCCCAAACU | 2447 |
| | GGUGCGGAU | |
| R5454_CasPhi12_S | AUUGCUCCUUACGAGGAGACUUCUCACU | 2448 |
| | CAGCGCAUC | |
| R5455_CasPhi12_S | AUUGCUCCUUACGAGGAGACAGCUGGGG | 2449 |
| | GAAGGUGGC | |
| R5456_CasPhi12_S | AUUGCUCCUUACGAGGAGACCCCCAGCU | 2450 |
| | GAAGUCCUU | |
| R5457_CasPhi12_S | AUUGCUCCUUACGAGGAGACCAAGGACU | 2451 |
| | UCAGCUGGG | |
| R5458_CasPhi12_S | AUUGCUCCUUACGAGGAGACCCAAGGAC | 2452 |
| | UUCAGCUGG | |
| R5459_CasPhi12_S | AUUGCUCCUUACGAGGAGACAGGGUUUC | 2453 |
| | CAAGGACUU | |
| R5460_CasPhi12_S | AUUGCUCCUUACGAGGAGACUAGGCACC | 2454 |
| | CAGGUCAGU | |
| R5461_CasPhi12_S | AUUGCUCCUUACGAGGAGACGUAGGCAC | 2455 |
| | CCAGGUCAG | |
| R5462_CasPhi12_S | AUUGCUCCUUACGAGGAGACGCUCGCUG | 2456 |
| | CAUCCCUGC | |
| R5463_CasPhi12_S | AUUGCUCCUUACGAGGAGACGCCUGAGC | 2457 |
| | AGGGAUGCA | |
| R5464_CasPhi12_S | AUUGCUCCUUACGAGGAGACUACAAUAA | 2458 |
| | CUGCAUCUG | |
| R5465_CasPhi12_S | AUUGCUCCUUACGAGGAGACGCUCGUGU | 2459 |
| | GCUUCCGGA | |
| R5466_CasPhi12_S | AUUGCUCCUUACGAGGAGACCGGACAUG | 2460 |
| | GUGUCCCUC | |
| R5467_CasPhi12_S | AUUGCUCCUUACGAGGAGACACGGCUGC | 2461 |
| | CGGGGCCCA | |
| R5468_CasPhi12_S | AUUGCUCCUUACGAGGAGACGGAGGUGU | 2462 |
| | CCUCAUGUG | |
| R5469_CasPhi12_S | AUUGCUCCUUACGAGGAGACCUGGACAC | 2463 |
| | UGAAUGGGA | |
| R5470_CasPhi12_S | AUUGCUCCUUACGAGGAGACAGUGUCCA | 2464 |
| | GGAACACCU | |
| R5471_CasPhi12_S | AUUGCUCCUUACGAGGAGACCAGGUGUU | 2465 |
| | CCUGGACAC | |
| R5472_CasPhi12_S | AUUGCUCCUUACGAGGAGACUUGCAGGU | 2466 |
| | GUUCCUGGA | |
| R5473_CasPhi12_S | AUUGCUCCUUACGAGGAGACACGGAUCA | 2467 |
| | GCCUGAGAU | |

TABLE AC
CasΦ.12 gRNAs targeting mouse PCSK9

| | | SEQ ID |
|---|---|---|
| Name | Repeat + spacer RNA Sequence (5′ → 3′) | NO |
| R4238_CasPhi12_S | AUUGCUCCUUACGAGGAGACCCGCUGUUGCCG | 1734 |
| | CCGCU | |
| R4239_CasPhi12_S | AUUGCUCCUUACGAGGAGACCCGCCGCUGCUG | 1735 |
| | CUGCU | |
| R4240_CasPhi12_S | AUUGCUCCUUACGAGGAGACCUGCUACUGUGC | 1736 |
| | CCCAC | |
| R4241_CasPhi12_S | AUUGCUCCUUACGAGGAGACAUAAUCUCCAUC | 1737 |
| | CUCGU | |
| R4242_CasPhi12_S | AUUGCUCCUUACGAGGAGACUGAAGAGCUGAU | 1738 |
| | GCUCG | |
| R4243_CasPhi12_S | AUUGCUCCUUACGAGGAGACGAGCAACGGCGG | 1739 |
| | AAGGU | |
| R4244_CasPhi12_S | AUUGCUCCUUACGAGGAGACCUGGCAGCCUCC | 1740 |
| | AGGCC | |
| R4245_CasPhi12_S | AUUGCUCCUUACGAGGAGACUGGUGCUGAUGG | 1741 |
| | AGGAG | |
| R4246_CasPhi12_S | AUUGCUCCUUACGAGGAGACAAUCUGUAGCCU | 1742 |
| | CUGGG | |
| R4247_CasPhi12_S | AUUGCUCCUUACGAGGAGACUUCAAUCUGUAG | 1743 |
| | CCUCU | |
| R4248_CasPhi12_S | AUUGCUCCUUACGAGGAGACGUUCAAUCUGUA | 1744 |
| | GCCUC | |
| R4249_CasPhi12_S | AUUGCUCCUUACGAGGAGACAACAAACUGCCC | 1745 |
| | ACCGC | |
| R4250_CasPhi12_S | AUUGCUCCUUACGAGGAGACAUGACAUAGCCC | 1746 |
| | CGGCG | |
| R4251_CasPhi12_S | AUUGCUCCUUACGAGGAGACUACAUAUCUUUU | 1747 |
| | AUGAC | |
| R4252_CasPhi12_S | AUUGCUCCUUACGAGGAGACUAUGACCUCUUC | 1748 |
| | CCUGG | |
| R4253_CasPhi12_S | AUUGCUCCUUACGAGGAGACAUGACCUCUUCC | 1749 |
| | CUGGC | |
| R4254_CasPhi12_S | AUUGCUCCUUACGAGGAGACUGACCUCUUCCC | 1750 |
| | UGGCU | |
| R4255_CasPhi12_S | AUUGCUCCUUACGAGGAGACACCAAGAAGCCA | 1751 |
| | GGGAA | |
| R4256_CasPhi12_S | AUUGCUCCUUACGAGGAGACCCUGGCUUCUUG | 1752 |
| | GUGAA | |
| R4257_CasPhi12_S | AUUGCUCCUUACGAGGAGACUUGGUGAAGAUG | 1753 |
| | AGCAG | |
| R4258_CasPhi12_S | AUUGCUCCUUACGAGGAGACGUGAAGAUGAGC | 1754 |
| | AGUGA | |
| R4259_CasPhi12_S | AUUGCUCCUUACGAGGAGACCCCCAUGUGGAG | 1755 |
| | UACAU | |
| R4260_CasPhi12_S | AUUGCUCCUUACGAGGAGACCUCAAUGUACUC | 1756 |
| | CACAU | |
| R4261_CasPhi12_S | AUUGCUCCUUACGAGGAGACAGGAAGACUCCU | 1757 |
| | UUGUC | |
| R4262_CasPhi12_S | AUUGCUCCUUACGAGGAGACGUCUUCGCCCAG | 1758 |
| | AGCAU | |
| R4263_CasPhi12_S | AUUGCUCCUUACGAGGAGACUCUUCGCCCAGA | 1759 |
| | GCAUC | |
| R4264_CasPhi12_S | AUUGCUCCUUACGAGGAGACGCCCAGAGCAUC | 1760 |
| | CCAUG | |
| R4265_CasPhi12_S | AUUGCUCCUUACGAGGAGACCAUGGGAUGCUC | 1761 |
| | UGGGC | |
| R4266_CasPhi12_S | AUUGCUCCUUACGAGGAGACGCUCCAGGUUCC | 1762 |
| | AUGGG | |
| R4267_CasPhi12_S | AUUGCUCCUUACGAGGAGACUCCCAGCAUGGC | 1763 |
| | ACCAG | |
| R4268_CasPhi12_S | AUUGCUCCUUACGAGGAGACCUCUGUCUGGUG | 1764 |
| | CCAUG | |
| R4269_CasPhi12_S | AUUGCUCCUUACGAGGAGACGAUACCAGCAUC | 1765 |
| | CAGGG | |
| R4270_CasPhi12_S | AUUGCUCCUUACGAGGAGACAGGGCAGGGUCA | 1766 |
| | CCAUC | |
| R4271_CasPhi12_S | AUUGCUCCUUACGAGGAGACAAGUCGGUGAUG | 1767 |
| | GUGAC | |
| R4272_CasPhi12_S | AUUGCUCCUUACGAGGAGACAACAGCGUGCCG | 1768 |
| | GAGGA | |
| R4273_CasPhi12_S | AUUGCUCCUUACGAGGAGACGCCACACCAGCA | 1769 |
| | UCCCG | |
| R4274_CasPhi12_S | AUUGCUCCUUACGAGGAGACAGCACACGCAGG | 1770 |
| | CUGUG | |
| R4275_CasPhi12_S | AUUGCUCCUUACGAGGAGACACAGUUGAGCAC | 1771 |
| | ACGCA | |
| R4276_CasPhi12_S | AUUGCUCCUUACGAGGAGACCCUUGACAGUUG | 1772 |
| | AGCAC | |
| R4277_CasPhi12_S | AUUGCUCCUUACGAGGAGACGCUGACUCUUCC | 1773 |
| | GAAUA | |
| R4278_CasPhi12_S | AUUGCUCCUUACGAGGAGACAUUCGGAAGAGU | 1774 |
| | CAGCU | |
| R4279_CasPhi12_S | AUUGCUCCUUACGAGGAGACUUCGGAAGAGUC | 1775 |
| | AGCUA | |
| R4280_CasPhi12_S | AUUGCUCCUUACGAGGAGACGGAAGAGUCAGC | 1776 |
| | UAAUC | |
| R4281_CasPhi12_S | AUUGCUCCUUACGAGGAGACUGCUGCCCCUGG | 1777 |
| | CCGGU | |
| R4282_CasPhi12_S | AUUGCUCCUUACGAGGAGACAGGAUGCGGCUA | 1778 |
| | UACCC | |
| R4283_CasPhi12_S | AUUGCUCCUUACGAGGAGACCCAGCUGCUGCA | 1779 |
| | ACCAG | |
| R4284_CasPhi12_S | AUUGCUCCUUACGAGGAGACCAGCAGCUGGGA | 1780 |
| | ACUUC | |
| R4285_CasPhi12_S | AUUGCUCCUUACGAGGAGACCGGGACGACGCC | 1781 |
| | UGCCU | |
| R4286_CasPhi12_S | AUUGCUCCUUACGAGGAGACGUGGCCCCGACU | 1782 |
| | GUGAU | |
| R4287_CasPhi12_S | AUUGCUCCUUACGAGGAGACCCUUGGGGACUU | 1783 |
| | UGGGG | |
| R4288_CasPhi12_S | AUUGCUCCUUACGAGGAGACGUCCCCAAAGUC | 1784 |
| | CCCAA | |
| R4289_CasPhi12_S | AUUGCUCCUUACGAGGAGACGGGACUUUGGGG | 1785 |
| | ACUAA | |
| R4290_CasPhi12_S | AUUGCUCCUUACGAGGAGACGGGGACUAAUUU | 1786 |
| | UGGAC | |
| R4291_CasPhi12_S | AUUGCUCCUUACGAGGAGACGGGACUAAUUUU | 1787 |
| | GGACG | |
| R4292_CasPhi12_S | AUUGCUCCUUACGAGGAGACUGGACGCUGUGU | 1788 |
| | GGAUC | |
| R4293_CasPhi12_S | AUUGCUCCUUACGAGGAGACGGACGCUGUGUG | 1789 |
| | GAUCU | |
| R4294_CasPhi12_S | AUUGCUCCUUACGAGGAGACGACGCUGUGUGG | 1790 |
| | AUCUC | |
| R4295_CasPhi12_S | AUUGCUCCUUACGAGGAGACCCGGGGGCAAAG | 1791 |
| | AGAUC | |
| R4296_CasPhi12_S | AUUGCUCCUUACGAGGAGACGCCCCCGGGAAG | 1792 |
| | GACAU | |
| R4297_CasPhi12_S | AUUGCUCCUUACGAGGAGACCCCCCGGGAAGG | 1793 |
| | ACAUC | |
| R4298_CasPhi12_S | AUUGCUCCUUACGAGGAGACAUGUCACAGAGU | 1794 |
| | GGGAC | |
| R4299_CasPhi12_S | AUUGCUCCUUACGAGGAGACUGGCUCGGAUGC | 1795 |
| | UGAGC | |
| R4300_CasPhi12_S | AUUGCUCCUUACGAGGAGACCCCUGGCCGAGC | 1796 |
| | UGCGG | |
| R4301_CasPhi12_S | AUUGCUCCUUACGAGGAGACGUAGAGAAGUGG | 1797 |
| | AUCAG | |
| R4302_CasPhi12_S | AUUGCUCCUUACGAGGAGACGGUAGAGAAGUG | 1798 |
| | GAUCA | |
| R4303_CasPhi12_S | AUUGCUCCUUACGAGGAGACUCUACCAAAGAC | 1799 |
| | GUCAU | |
| R4304_CasPhi12_S | AUUGCUCCUUACGAGGAGACAUGACGUCUUUG | 1800 |
| | GUAGA | |
| R4305_CasPhi12_S | AUUGCUCCUUACGAGGAGACCCUGAGGACCAG | 1801 |
| | CAGGU | |
| R4306_CasPhi12_S | AUUGCUCCUUACGAGGAGACGGGGUCAGCACC | 1802 |
| | UGCUG | |
| R4307_CasPhi12_S | AUUGCUCCUUACGAGGAGACGAGUGGGCCCCG | 1803 |
| | AGUGU | |
| R4308_CasPhi12_S | AUUGCUCCUUACGAGGAGACUGGGGCACAGCG | 1804 |
| | GGCUG | |
| R4309_CasPhi12_S | AUUGCUCCUUACGAGGAGACUCCAGGAGCGGG | 1805 |
| | AGGCG | |
| R4310_CasPhi12_S | AUUGCUCCUUACGAGGAGACCAGACCUGCUGG | 1806 |
| | CCUCC | |
| R4311_CasPhi12_S | AUUGCUCCUUACGAGGAGACAGGGCCUUGCAG | 1807 |
| | ACCUG | |
| R4312_CasPhi12_S | AUUGCUCCUUACGAGGAGACGGGGGUGAGGGU | 1808 |
| | GUCUA | |
| R4313_CasPhi12_S | AUUGCUCCUUACGAGGAGACGGGGUGAGGGUG | 1809 |
| | UCUAU | |
| R4314_CasPhi12_S | AUUGCUCCUUACGAGGAGACGCACGGGGAACC | 1810 |
| | AGGCA | |
| R4315_CasPhi12_S | AUUGCUCCUUACGAGGAGACCCCGUGCCAACU | 1811 |
| | GCAGC | |
| R4316_CasPhi12_S | AUUGCUCCUUACGAGGAGACUGGAUGCUGCAG | 1812 |
| | UUGGC | |
| R4317_CasPhi12_S | AUUGCUCCUUACGAGGAGACUGGUGGCAGUGG | 1813 |
| | ACAUG | |
| R4318_CasPhi12_S | AUUGCUCCUUACGAGGAGACCACUUCCCAAUG | 1814 |
| | GAAGC | |
| R4319_CasPhi12_S | AUUGCUCCUUACGAGGAGACCAUUGGGAAGUG | 1815 |
| | GAAGA | |
| R4320_CasPhi12_S | AUUGCUCCUUACGAGGAGACGGAAGUGGAAGA | 1816 |
| | CCUUA | |
| R4321_CasPhi12_S | AUUGCUCCUUACGAGGAGACGUGUCCGGAGGC | 1817 |
| | AGCCU | |
| R4322_CasPhi12_S | AUUGCUCCUUACGAGGAGACGCCACCAGGCGG | 1818 |
| | CCAGU | |
| R4323_CasPhi12_S | AUUGCUCCUUACGAGGAGACCUGCUGCCAUGC | 1819 |
| | CCCAG | |
| R4324_CasPhi12_S | AUUGCUCCUUACGAGGAGACCAGCCCUGGGGC | 1820 |
| | AUGGC | |
| R4325_CasPhi12_S | AUUGCUCCUUACGAGGAGACCAUUCCAGCCCU | 1821 |
| | GGGGC | |
| R4326_CasPhi12_S | AUUGCUCCUUACGAGGAGACGCAUUCCAGCCC | 1822 |
| | UGGGG | |
| R4327_CasPhi12_S | AUUGCUCCUUACGAGGAGACUGCAUUCCAGCC | 1823 |
| | CUGGG | |
| R4328_CasPhi12_S | AUUGCUCCUUACGAGGAGACAUUUUGCAUUCC | 1824 |
| | AGCCC | |
| R4329_CasPhi12_S | AUUGCUCCUUACGAGGAGACCAUCCAGUCAGG | 1825 |
| | GUCCA | |
| R4330_CasPhi12_S | AUUGCUCCUUACGAGGAGACUCCACGCUGUAG | 1826 |
| | GCUCC | |
| R4331_CasPhi12_S | AUUGCUCCUUACGAGGAGACCCACACACAGGU | 1827 |
| | UGUCC | |
| R4332_CasPhi12_S | AUUGCUCCUUACGAGGAGACUCCACUGGUCCU | 1828 |
| | GUCUG | |
| R4333_CasPhi12_S | AUUGCUCCUUACGAGGAGACCUGAAGGCCGGC | 1829 |
| | UCCGG | |

TABLE AD
CasΦ.12 gRNAs targeting Bak1 in CHO cells

| | Repeat + spacer RNA Sequence (5′ → 3′), | SEQ ID |
|---|---|---|
| Name | shown as DNA | NO |
| R2452 | ATTGCTCCTTACGAGGAGACGAAGCTATGTT | 1830 |
| Bak1_CasPhi12_1_S | TTCCAT | |
| R2453 | ATTGCTCCTTACGAGGAGACGCAGGGGCAGC | 1831 |
| Bak1_CasPhi12_2_S | CGCCCC | |
| R2454 | ATTGCTCCTTACGAGGAGACCTCCTAGAACC | 1832 |
| Bak1_CasPhi12_3_S | CAACAG | |
| R2455 | ATTGCTCCTTACGAGGAGACGAAAGACCTCC | 1833 |
| Bak1_CasPhi12_4_S | TCTGTG | |
| R2456 | ATTGCTCCTTACGAGGAGACTCCATCTCGGG | 1834 |
| Bak1_CasPhi12_5_S | GTTGGC | |
| R2457 | ATTGCTCCTTACGAGGAGACTTCCTGATGGT | 1835 |
| Bak1_CasPhi12_6_S | GGAGAT | |
| R2849_Bak1_CasPhi12_ | ATTGCTCCTTACGAGGAGACCTGACTCCCAG | 1836 |
| nsd_sg1_S | CTCTGA | |
| R2850_Bak1_ | ATTGCTCCTTACGAGGAGACTGGGGTCAGAG | 1837 |
| CasPhi12_nsd_sg2_S | CTGGGA | |
| R2851_Bak1_CasPhi12_ | ATTGCTCCTTACGAGGAGACGAAAGACCTCC | 1838 |
| nsd_sg3_S | TCTGTG | |
| R2852_Bak1_ | ATTGCTCCTTACGAGGAGACCGAAGCTATGT | 1839 |
| CasPhi12_nsd_sg4_S | TTTCCA | |
| R2853_Bak1_ | ATTGCTCCTTACGAGGAGACGAAGCTATGTT | 1840 |
| CasPhi12_nsd_sg5_S | TTCCAT | |
| R2854_Bak1_ | ATTGCTCCTTACGAGGAGACTCCATCTCCACC | 1841 |
| CasPhi12_nsd_sg6_S | ATCAG | |
| R2855_Bak1_ | ATTGCTCCTTACGAGGAGACCCATCTCCACC | 1842 |
| CasPhi12_nsd_sg7_S | ATCAGG | |
| R2856_Bak1_ | ATTGCTCCTTACGAGGAGACCTGATGGTGGA | 1843 |
| CasPhi12_nsd_sg8_S | GATGGA | |
| R2857_Bak1_ | ATTGCTCCTTACGAGGAGACCATCTCCACCA | 1844 |
| CasPhi12_nsd_sg9_S | TCAGGA | |
| R2858_Bak1_ | ATTGCTCCTTACGAGGAGACTTCCTGATGGT | 1845 |
| CasPhi12_nsd_sg10_S | GGAGAT | |
| R2859_Bak1_ | ATTGCTCCTTACGAGGAGACGCAGGGGCAGC | 1846 |
| CasPhi12_nsd_sg11_S | CGCCCC | |
| R2860_Bak1_ | ATTGCTCCTTACGAGGAGACTCCATCTCGGG | 1847 |
| CasPhi12_nsd_sg12_S | GTTGGC | |
| R2861_Bak1_ | ATTGCTCCTTACGAGGAGACTAGGAGCAAAT | 1848 |
| CasPhi12_nsd_sg13_S | TGTCCA | |
| R2862_Bak1_ | ATTGCTCCTTACGAGGAGACGGTTCTAGGAG | 1849 |
| CasPhi12_nsd_sg14_S | CAAATT | |
| R2863_Bak1_ | ATTGCTCCTTACGAGGAGACGCTCCTAGAAC | 1850 |
| CasPhi12_nsd_sg15_S | CCAACA | |
| R2864_Bak1_ | ATTGCTCCTTACGAGGAGACCTCCTAGAACC | 1851 |
| CasPhi12_nsd_sg16_S | CAACAG | |
| R3977_Bak1_ | ATTGCTCCTTACGAGGAGACTCCAGACGCCA | 1852 |
| CasPhi12_exon1_sg1_ | TCTTTC | |
| S | | |
| R3978_Bak1_ | ATTGCTCCTTACGAGGAGACTGGTAAGAGTC | 1853 |
| CasPhi12_exon1_sg2_ | CTCCTG | |
| S | | |
| R3979_Bak1_ | ATTGCTCCTTACGAGGAGACTTACAGCATCTT | 1854 |
| CasPhi12_exon3_sg1_ | GGGTC | |
| S | | |
| R3980_Bak1_ | ATTGCTCCTTACGAGGAGACGGTCAGGTGGG | 1855 |
| CasPhi12_exon3_sg2_ | CCGGCA | |
| S | | |
| R3981_Bak1_ | ATTGCTCCTTACGAGGAGACCTATCATTGGA | 1856 |
| CasPhi12_exon3_sg3_ | GATGAC | |
| S | | |
| R3982_Bak1_ | ATTGCTCCTTACGAGGAGACGAGATGACATT | 1857 |
| CasPhi12_exon3_sg4_ | AACCGG | |
| S | | |
| R3983_Bak1_ | ATTGCTCCTTACGAGGAGACTGGAACTCTGT | 1858 |
| CasPhi12_exon3 sg5_ | GTCGTA | |
| S | | |
| R3984_Bak1_ | ATTGCTCCTTACGAGGAGACCAGAATTTACT | 1859 |
| CasPhi12_exon3_sg6_ | GGAGCA | |
| S | | |
| R3985_Bak1_ | ATTGCTCCTTACGAGGAGACACTGGAGCAGC | 1860 |
| CasPhi12_exon3_sg7_ | TGCAGC | |
| S | | |
| R3986_Bak1_ | ATTGCTCCTTACGAGGAGACCCAGCTGTGGG | 1861 |
| CasPhi12_exon3_sg8_ | CTGCAG | |
| S | | |
| R3987_Bak1_ | ATTGCTCCTTACGAGGAGACGTAGGCATTCC | 1862 |
| CasPhi12_exon3_sg9_ | CAGCTG | |
| S | | |
| R3988_Bak1_ | ATTGCTCCTTACGAGGAGACGTGAAGAGTTC | 1863 |
| CasPhi12_exon3_sg10_ | GTAGGC | |
| S | | |
| R3989_Bak1_ | ATTGCTCCTTACGAGGAGACACCAAGATTGC | 1864 |
| CasPhi12_exon3_sg11_ | CTCCAG | |
| S | | |
| R3990_Bak1_ | ATTGCTCCTTACGAGGAGACCCTCCAGGTAC | 1865 |
| CasPhi12_exon3_sg12_ | CCACCA | |
| S | | |

TABLE AE
CasΦ.12 gRNAs targeting Bax in CHO cells

| | Repeat + spacer RNA Sequence (5′ → 3′), | SEQ ID |
|---|---|---|
| Name | shown as DNA) | NO |
| R2458 | ATTGCTCCTTACGAGGAGACCTAATGTGGAT | 1866 |
| Bax_CasPhi12_1_S | ACTAAC | |
| R2459 | ATTGCTCCTTACGAGGAGACTTCCGTGTGGC | 1867 |
| Bax_CasPhi12_2_S | AGCTGA | |
| R2460 | ATTGCTCCTTACGAGGAGACCTGATGGCAAC | 1868 |
| Bax_CasPhi12_3_S | TTCAAC | |
| R2461 | ATTGCTCCTTACGAGGAGACTACTTTGCTAGC | 1869 |
| Bax_CasPhi12_4_S | AAACT | |
| R2462 | ATTGCTCCTTACGAGGAGACAGCACCAGTTT | 1870 |
| Bax_CasPhi12_5_S | GCTAGC | |
| R2463 | ATTGCTCCTTACGAGGAGACAACTGGGGCCG | 1871 |
| Bax_CasPhi12_6_S | GGTTGT | |
| R2865_Bax_CasPhi12_ | ATTGCTCCTTACGAGGAGACTTCTCTTTCCTG | 1872 |
| nsd_sg1_S | TAGGA | |
| R2866_Bax_CasPhi12_ | ATTGCTCCTTACGAGGAGACTCTTTCCTGTAG | 1873 |
| nsd_sg2_S | GATGA | |
| R2867_Bax_ | ATTGCTCCTTACGAGGAGACCCTGTAGGATG | 1874 |
| CasPhi12_nsd_sg3_S | ATTGCT | |
| R2868_Bax_ | ATTGCTCCTTACGAGGAGACCTGTAGGATGA | 1875 |
| CasPhi12_nsd_sg4_S | TTGCTA | |
| R2869_Bax_ | ATTGCTCCTTACGAGGAGACCTAATGTGGAT | 1876 |
| CasPhi12_nsd_sg5_S | ACTAAC | |
| R2870_Bax_ | ATTGCTCCTTACGAGGAGACTTCCGTGTGGC | 1877 |
| CasPhi12_nsd_sg6_S | AGCTGA | |
| R2871_Bax_ | ATTGCTCCTTACGAGGAGACCGTGTGGCAGC | 1878 |
| CasPhi12_nsd_sg7_S | TGACAT | |
| R2872_Bax_ | ATTGCTCCTTACGAGGAGACCCATCAGCAAA | 1879 |
| CasPhi12_nsd_sg8_S | CATGTC | |
| R2873_Bax_ | ATTGCTCCTTACGAGGAGACAAGTTGCCATC | 1880 |
| CasPhi12_nsd_sg9_S | AGCAAA | |
| R2874_Bax_ | ATTGCTCCTTACGAGGAGACGCTGATGGCAA | 1881 |
| CasPhi12_nsd_sg10_S | CTTCAA | |
| R2875_Bax_ | ATTGCTCCTTACGAGGAGACCTGATGGCAAC | 1882 |
| CasPhi12_nsd_sg11_S | TTCAAC | |
| R2876_Bax_ | ATTGCTCCTTACGAGGAGACAACTGGGGCCG | 1883 |
| CasPhi12_nsd_sg12_S | GGTTGT | |
| R2877_Bax_ | ATTGCTCCTTACGAGGAGACTTGCCCTTTTCT | 1884 |
| CasPhi12_nsd_sg13_S | ACTTT | |
| R2878_Bax_ | ATTGCTCCTTACGAGGAGACCCCTTTTCTACT | 1885 |
| CasPhi12_nsd_sg14_S | TTGCT | |
| R2879_Bax_ | ATTGCTCCTTACGAGGAGACCTAGCAAAGTA | 1886 |
| CasPhi12_nsd_sg15_S | GAAAAG | |
| R2880_Bax_ | ATTGCTCCTTACGAGGAGACGCTAGCAAAGT | 1887 |
| CasPhi12_nsd_sg16_S | AGAAAA | |
| R2881_Bax_ | ATTGCTCCTTACGAGGAGACTCTACTTTGCTA | 1888 |
| CasPhi12_nsd_sg17_S | GCAAA | |
| R2882_Bax_ | ATTGCTCCTTACGAGGAGACCTACTTTGCTAG | 1889 |
| CasPhi12_nsd_sg18_S | CAAAC | |
| R2883_Bax_ | ATTGCTCCTTACGAGGAGACTACTTTGCTAGC | 1890 |
| CasPhi12_nsd_sg19_S | AAACT | |
| R2884_Bax_ | ATTGCTCCTTACGAGGAGACGCTAGCAAACT | 1891 |
| CasPhi12_nsd_sg20_S | GGTGCT | |
| R2885_Bax_ | ATTGCTCCTTACGAGGAGACCTAGCAAACTG | 1892 |
| CasPhi12_nsd_sg21_S | GTGCTC | |
| R2886_Bax_ | ATTGCTCCTTACGAGGAGACAGCACCAGTTT | 1893 |
| CasPhi12_nsd_sg22_S | GCTAGC | |

TABLE AF
CasΦ.12 gRNAs targeting Fut8 in CHO cells

| | Repeat + spacer RNA Sequence (5′ → 3′), | SEQ ID |
|---|---|---|
| Name | shown as DNA) | NO |
| R2464 | ATTGCTCCTTACGAGGAGACCCACTTTGTCA | 1894 |
| Fut8_CasPhi12_1_S | GTGCGT | |
| R2465 | ATTGCTCCTTACGAGGAGACCTCAATGGGAT | 1895 |
| Fut8_CasPhi12_2_S | GGAAGG | |
| R2466 | ATTGCTCCTTACGAGGAGACAGGAATACATG | 1896 |
| Fut8_CasPhi12_3_S | GTACAC | |
| R2467 | ATTGCTCCTTACGAGGAGACAAGAACATTTT | 1897 |
| Fut8_CasPhi12_4_S | CAGCTT | |
| R2468 | ATTGCTCCTTACGAGGAGACATCCACTTTCAT | 1898 |
| Fut8_CasPhi12_5_S | TCTGC | |
| R2469 | ATTGCTCCTTACGAGGAGACTTTGTTAAAGG | 1899 |
| Fut8_CasPhi12_6_S | AGGCAA | |
| R2887_Fut8_CasPhi12_ | ATTGCTCCTTACGAGGAGACTCCCCAGAGTC | 1900 |
| nsd_sg1_S | CATGTC | |
| R2888_Fut8 | ATTGCTCCTTACGAGGAGACTCAGTGCGTCT | 1901 |
| CasPhi12_nsd_sg2_S | GACATG | |
| R2889_Fut8_CasPhi12_ | ATTGCTCCTTACGAGGAGACGTCAGTGCGTC | 1902 |
| nsd_sg3_S | TGACAT | |
| R2890_Fut8 | ATTGCTCCTTACGAGGAGACCCACTTTGTCA | 1903 |
| CasPhi12_nsd_sg4_S | GTGCGT | |
| R2891_Fut8 | ATTGCTCCTTACGAGGAGACTGTTCCCACTTT | 1904 |
| CasPhi12_nsd_sg5_S | GTCAG | |
| R2892_Fut8 | ATTGCTCCTTACGAGGAGACCTCAATGGGAT | 1905 |
| CasPhi12_nsd_sg6_S | GGAAGG | |
| R2893_Fut8 | ATTGCTCCTTACGAGGAGACCATCCCATTGA | 1906 |
| CasPhi12_nsd_sg7_S | GGAATA | |
| R2894_Fut8 | ATTGCTCCTTACGAGGAGACAGGAATACATG | 1907 |
| CasPhi12_nsd_sg8_S | GTACAC | |
| R2895_Fut8 | ATTGCTCCTTACGAGGAGACAACGTGTACCA | 1908 |
| CasPhi12_nsd_sg9_S | TGTATT | |
| R2896_Fut8 | ATTGCTCCTTACGAGGAGACTTCAACGTGTA | 1909 |
| CasPhi12_nsd_sg10_S | CCATGT | |
| R2897_Fut8 | ATTGCTCCTTACGAGGAGACAAGAACATTTT | 1910 |
| CasPhi12_nsd_sg11_S | CAGCTT | |
| R2898_Fut8 | ATTGCTCCTTACGAGGAGACGAGAAGCTGAA | 1911 |
| CasPhi12_nsd_sg12_S | AATGTT | |
| R2899_Fut8 | ATTGCTCCTTACGAGGAGACTCAGCTTCTCG | 1912 |
| CasPhi12_nsd_sg13_S | AACGCA | |
| R2900_Fut8 | ATTGCTCCTTACGAGGAGACCAGCTTCTCGA | 1913 |
| CasPhi12_nsd_sg14_S | ACGCAG | |
| R2901_Fut8 | ATTGCTCCTTACGAGGAGACTGCGTTCGAGA | 1914 |
| CasPhi12_nsd_sg15_S | AGCTGA | |
| R2902_Fut8 | ATTGCTCCTTACGAGGAGACAGCTTCTCGAA | 1915 |
| CasPhi12_nsd_sg16_S | CGCAGA | |
| R2903_Fut8 | ATTGCTCCTTACGAGGAGACATTCTGCGTTCG | 1916 |
| CasPhi12_nsd_sg17_S | AGAAG | |
| R2904_Fut8 | ATTGCTCCTTACGAGGAGACCATTCTGCGTTC | 1917 |
| CasPhi12_nsd_sg18_S | GAGAA | |
| R2905_Fut8 | ATTGCTCCTTACGAGGAGACTCGAACGCAGA | 1918 |
| CasPhi12_nsd_sg19_S | ATGAAA | |
| R2906_Fut8 | ATTGCTCCTTACGAGGAGACATCCACTTTCAT | 1919 |
| CasPhi12_nsd_sg20_S | TCTGC | |
| R2907_Fut8 | ATTGCTCCTTACGAGGAGACTATCCACTTTCA | 1920 |
| CasPhi12_nsd_sg21_S | TTCTG | |
| R2908_Fut8 | ATTGCTCCTTACGAGGAGACTTATCCACTTTC | 1921 |
| CasPhi12_nsd_sg22_S | ATTCT | |
| R2909_Fut8 | ATTGCTCCTTACGAGGAGACTTTATCCACTTT | 1922 |
| CasPhi12_nsd_sg23_S | CATTC | |
| R2910_Fut8 | ATTGCTCCTTACGAGGAGACTTTTATCCACTT | 1923 |
| CasPhi12_nsd_sg24_S | TCATT | |
| R2911_Fut8 | ATTGCTCCTTACGAGGAGACAACAAAGAAGG | 1924 |
| CasPhi12_nsd_sg25_S | GTCATC | |
| R2912_Fut8 | ATTGCTCCTTACGAGGAGACCCTCCTTTAACA | 1925 |
| CasPhi12_nsd_sg26_S | AAGAA | |
| R2913_Fut8 | ATTGCTCCTTACGAGGAGACGCCTCCTTTAAC | 1926 |
| CasPhi12_nsd_sg27_S | AAAGA | |
| R2914_Fut8 | ATTGCTCCTTACGAGGAGACTTTGTTAAAGG | 1927 |
| CasPhi12_nsd_sg28_S | AGGCAA | |
| R2915_Fut8 | ATTGCTCCTTACGAGGAGACGTTAAAGGAGG | 1928 |
| CasPhi12_nsd_sg29_S | CAAAGA | |
| R2916_Fut8 | ATTGCTCCTTACGAGGAGACTTAAAGGAGGC | 1929 |
| CasPhi12_nsd_sg30_S | AAAGAC | |
| R2917_Fut8 | ATTGCTCCTTACGAGGAGACTCTTTGCCTCCT | 1930 |
| CasPhi12_nsd_sg31_S | TTAAC | |
| R2918_Fut8 | ATTGCTCCTTACGAGGAGACGTCTTTGCCTCC | 1931 |
| CasPhi12_nsd_sg32_S | TTTAA | |
| R2919_Fut8 | ATTGCTCCTTACGAGGAGACGTCTAACTTACT | 1932 |
| CasPhi12_nsd_sg33_S | TTGTC | |
| R2920_Fut8 | ATTGCTCCTTACGAGGAGACTTGGTCTAACTT | 1933 |
| CasPhi12_nsd_sg34_S | ACTTT | |

TABLE AG
CasΦ.12 gRNAs targeting Fut8

| | | | Repeat | | |
|---|---|---|---|---|---|
| | Repeat | Spacer | sequence | Spacer sequence | crRNA sequence |
| Name | length | length | (5′ → 3′) | (5′ → 3′) | (5′ → 3′) |
| R3582 | 36 | 30 | CUUUCAAGA | AGGAAUACAU | CUUUCAAGACUAA |
| | | | CUAAUAGAU | GGUACACGUU | UAGAUUGCUCCUU |
| | | | UGCUCCUUA | GAAGAACAUU | ACGAGGAGACAGG |
| | | | CGAGGAGAC | (SEQ ID NO: | AAUACAUGGUACA |
| | | | (SEQ ID NO: | 1482) | CGUUGAAGAACAU |
| | | | 2469) | | U (SEQ ID NO: 1499) |
| R3583 | 36 | 29 | CUUUCAAGA | AGGAAUACAU | CUUUCAAGACUAA |
| | | | CUAAUAGAU | GGUACACGUU | UAGAUUGCUCCUU |
| | | | UGCUCCUUA | GAAGAACAU | ACGAGGAGACAGG |
| | | | CGAGGAGAC | (SEQ ID NO: | AAUACAUGGUACA |
| | | | (SEQ ID NO: | 1483) | CGUUGAAGAACAU |
| | | | 2469) | | (SEQ ID NO: 1500) |
| R3584 | 36 | 28 | CUUUCAAGA | AGGAAUACAU | CUUUCAAGACUAA |
| | | | CUAAUAGAU | GGUACACGUU | UAGAUUGCUCCUU |
| | | | UGCUCCUUA | GAAGAACA | ACGAGGAGACAGG |
| | | | CGAGGAGAC | (SEQ ID NO: | AAUACAUGGUACA |
| | | | (SEQ ID NO: | 1484) | CGUUGAAGAACA |
| | | | 2469) | | (SEQ ID NO: 1501) |
| R3585 | 36 | 27 | CUUUCAAGA | AGGAAUACAU | CUUUCAAGACUAA |
| | | | CUAAUAGAU | GGUACACGUU | UAGAUUGCUCCUU |
| | | | UGCUCCUUA | GAAGAAC | ACGAGGAGACAGG |
| | | | CGAGGAGAC | (SEQ ID NO: | AAUACAUGGUACA |
| | | | (SEQ ID NO: | 1485) | CGUUGAAGAAC |
| | | | 2469) | | (SEQ ID NO: 1502) |
| R3586 | 36 | 26 | CUUUCAAGA | AGGAAUACAU | CUUUCAAGACUAA |
| | | | CUAAUAGAU | GGUACACGUU | UAGAUUGCUCCUU |
| | | | UGCUCCUUA | GAAGAA (SEQ | ACGAGGAGACAGG |
| | | | CGAGGAGAC | ID NO: 1486) | AAUACAUGGUACA |
| | | | (SEQ ID NO: | | CGUUGAAGAA (SEQ |
| | | | 2469) | | ID NO: 1503) |
| R3587 | 36 | 25 | CUUUCAAGA | AGGAAUACAU | CUUUCAAGACUAA |
| | | | CUAAUAGAU | GGUACACGUU | UAGAUUGCUCCUU |
| | | | UGCUCCUUA | GAAGA (SEQ | ACGAGGAGACAGG |
| | | | CGAGGAGAC | ID NO: 1487) | AAUACAUGGUACA |
| | | | (SEQ ID NO: | | CGUUGAAGA (SEQ |
| | | | 2469) | | ID NO: 1504) |
| R3588 | 36 | 24 | CUUUCAAGA | AGGAAUACAU | CUUUCAAGACUAA |
| | | | CUAAUAGAU | GGUACACGUU | UAGAUUGCUCCUU |
| | | | UGCUCCUUA | GAAG (SEQ ID | ACGAGGAGACAGG |
| | | | CGAGGAGAC | NO: 1488) | AAUACAUGGUACA |
| | | | (SEQ ID NO: | | CGUUGAAG (SEQ ID |
| | | | 2469) | | NO: 1505) |
| R3589 | 36 | 23 | CUUUCAAGA | AGGAAUACAU | CUUUCAAGACUAA |
| | | | CUAAUAGAU | GGUACACGUU | UAGAUUGCUCCUU |
| | | | UGCUCCUUA | GAA (SEQ ID | ACGAGGAGACAGG |
| | | | CGAGGAGAC | NO: 1489) | AAUACAUGGUACA |
| | | | (SEQ ID NO: | | CGUUGAA (SEQ ID |
| | | | 2469) | | NO: 1506) |
| R3590 | 36 | 22 | CUUUCAAGA | AGGAAUACAU | CUUUCAAGACUAA |
| | | | CUAAUAGAU | GGUACACGUU | UAGAUUGCUCCUU |
| | | | UGCUCCUUA | GA (SEQ ID NO: | ACGAGGAGACAGG |
| | | | CGAGGAGAC | 1490) | AAUACAUGGUACA |
| | | | (SEQ ID NO: | | CGUUGA (SEQ ID |
| | | | 2469) | | NO: 1507) |
| R3591 | 36 | 21 | CUUUCAAGA | AGGAAUACAU | CUUUCAAGACUAA |
| | | | CUAAUAGAU | GGUACACGUU | UAGAUUGCUCCUU |
| | | | UGCUCCUUA | G (SEQ ID NO: | ACGAGGAGACAGG |
| | | | CGAGGAGAC | 1491) | AAUACAUGGUACA |
| | | | (SEQ ID NO: | | CGUUG (SEQ ID |
| | | | 2469) | | NO: 1508) |
| R3592 | 36 | 20 | CUUUCAAGA | AGGAAUACAU | CUUUCAAGACUAA |
| | | | CUAAUAGAU | GGUACACGUU | UAGAUUGCUCCUU |
| | | | UGCUCCUUA | (SEQ ID NO: | ACGAGGAGACAGG |
| | | | CGAGGAGAC | 1492) | AAUACAUGGUACA |
| | | | (SEQ ID NO: | | CGUU (SEQ ID |
| | | | 2469) | | NO: 1509) |
| R3593 | 36 | 19 | CUUUCAAGA | AGGAAUACAU | CUUUCAAGACUAA |
| | | | CUAAUAGAU | GGUACACGU | UAGAUUGCUCCUU |
| | | | UGCUCCUUA | (SEQ ID NO: | ACGAGGAGACAGG |
| | | | CGAGGAGAC | 1493) | AAUACAUGGUACA |
| | | | (SEQ ID NO: | | CGU (SEQ ID |
| | | | 2469) | | NO: 1510) |
| R3594 | 36 | 18 | CUUUCAAGA | AGGAAUACAU | CUUUCAAGACUAA |
| | | | CUAAUAGAU | GGUACACG | UAGAUUGCUCCUU |
| | | | UGCUCCUUA | (SEQ ID NO: | ACGAGGAGACAGG |
| | | | CGAGGAGAC | 1494) | AAUACAUGGUACA |
| | | | (SEQ ID NO: | | CG (SEQ ID NO: 1511) |
| | | | 2469) | | |
| R3595 | 36 | 17 | CUUUCAAGA | AGGAAUACAU | CUUUCAAGACUAA |
| | | | CUAAUAGAU | GGUACAC | UAGAUUGCUCCUU |
| | | | UGCUCCUUA | (SEQ ID NO: | ACGAGGAGACAGG |
| | | | CGAGGAGAC | 1495) | AAUACAUGGUACA |
| | | | (SEQ ID NO: | | C (SEQ ID NO: 1512) |
| | | | 2469) | | |
| R3596 | 36 | 16 | CUUUCAAGA | AGGAAUACAU | CUUUCAAGACUAA |
| | | | CUAAUAGAU | GGUACA (SEQ | UAGAUUGCUCCUU |
| | | | UGCUCCUUA | ID NO: 1496) | ACGAGGAGACAGG |
| | | | CGAGGAGAC | | AAUACAUGGUACA |
| | | | (SEQ ID NO: | | (SEQ ID NO: 1513) |
| | | | 2469) | | |
| R3597 | 36 | 15 | CUUUCAAGA | AGGAAUACAU | CUUUCAAGACUAA |
| | | | CUAAUAGAU | GGUAC (SEQ ID | UAGAUUGCUCCUU |
| | | | UGCUCCUUA | NO: 1497) | ACGAGGAGACAGG |
| | | | CGAGGAGAC | | AAUACAUGGUAC |
| | | | (SEQ ID NO: | | (SEQ ID NO: 1514) |
| | | | 2469) | | |
| R3598 | 35 | 20 | UUUCAAGAC | AGGAAUACAU | UUUCAAGACUAAU |
| | | | UAAUAGAUU | GGUACACGUU | AGAUUGCUCCUUA |
| | | | GCUCCUUAC | (SEQ ID NO: | CGAGGAGACAGGA |
| | | | GAGGAGAC | 1498) | AUACAUGGUACAC |
| | | | (SEQ ID NO: | | GUU (SEQ ID |
| | | | 1466) | | NO: 1515) |
| R3599 | 34 | 20 | UUCAAGACU | AGGAAUACAU | UUCAAGACUAAUA |
| | | | AAUAGAUUG | GGUACACGUU | GAUUGCUCCUUAC |
| | | | CUCCUUACG | (SEQ ID NO: | GAGGAGACAGGAA |
| | | | AGGAGAC | 1498) | UACAUGGUACACG |
| | | | (SEQ ID NO: | | UU (SEQ ID NO: 1516) |
| | | | 1467) | | |
| R3600 | 33 | 20 | UCAAGACUA | AGGAAUACAU | UCAAGACUAAUAG |
| | | | AUAGAUUGC | GGUACACGUU | AUUGCUCCUUACG |
| | | | UCCUUACGA | (SEQ ID NO: | AGGAGACAGGAAU |
| | | | GGAGAC (SEQ | 1498) | ACAUGGUACACGU |
| | | | ID NO: 1468) | | U (SEQ ID NO: 1517) |
| R3601 | 32 | 20 | CAAGACUAA | AGGAAUACAU | CAAGACUAAUAGA |
| | | | UAGAUUGCU | GGUACACGUU | UUGCUCCUUACGA |
| | | | CCUUACGAG | (SEQ ID NO: | GGAGACAGGAAUA |
| | | | GAGAC (SEQ | 1498) | CAUGGUACACGUU |
| | | | ID NO: 1469) | | (SEQ ID NO: 1518) |
| R3602 | 31 | 20 | AAGACUAAU | AGGAAUACAU | AAGACUAAUAGAU |
| | | | AGAUUGCUC | GGUACACGUU | UGCUCCUUACGAG |
| | | | CUUACGAGG | (SEQ ID NO: | GAGACAGGAAUAC |
| | | | AGAC (SEQ ID | 1498) | AUGGUACACGUU |
| | | | NO: 1470) | | (SEQ ID NO: 1519) |
| R3603 | 30 | 20 | AGACUAAUA | AGGAAUACAU | AGACUAAUAGAUU |
| | | | GAUUGCUCC | GGUACACGUU | GCUCCUUACGAGG |
| | | | UUACGAGGA | (SEQ ID NO: | AGACAGGAAUACA |
| | | | GAC (SEQ ID | 1498) | UGGUACACGUU |
| | | | NO: 1471) | | (SEQ ID NO: 1520) |
| R3604 | 29 | 20 | GACUAAUAG | AGGAAUACAU | GACUAAUAGAUUG |
| | | | AUUGCUCCU | GGUACACGUU | CUCCUUACGAGGA |
| | | | UACGAGGAG | (SEQ ID NO: | GACAGGAAUACAU |
| | | | AC (SEQ ID | 1498) | GGUACACGUU (SEQ |
| | | | NO: 1472) | | ID NO: 1521) |
| R3605 | 28 | 20 | ACUAAUAGA | AGGAAUACAU | ACUAAUAGAUUGC |
| | | | UUGCUCCUU | GGUACACGUU | UCCUUACGAGGAG |
| | | | ACGAGGAGA | (SEQ ID NO: | ACAGGAAUACAUG |
| | | | C (SEQ ID NO: | 1498) | GUACACGUU (SEQ |
| | | | 1473) | | ID NO: 1522) |
| R3606 | 27 | 20 | CUAAUAGAU | AGGAAUACAU | CUAAUAGAUUGCU |
| | | | UGCUCCUUA | GGUACACGUU | CCUUACGAGGAGA |
| | | | CGAGGAGAC | (SEQ ID NO: | CAGGAAUACAUGG |
| | | | (SEQ ID NO: | 1498) | UACACGUU (SEQ ID |
| | | | 1474) | | NO: 1523) |
| R3607 | 26 | 20 | UAAUAGAUU | AGGAAUACAU | UAAUAGAUUGCUC |
| | | | GCUCCUUAC | GGUACACGUU | CUUACGAGGAGAC |
| | | | GAGGAGAC | (SEQ ID NO: | AGGAAUACAUGGU |
| | | | (SEQ ID NO: | 1498) | ACACGUU (SEQ ID |
| | | | 1475) | | NO: 1524) |
| R3608 | 25 | 20 | AAUAGAUUG | AGGAAUACAU | AAUAGAUUGCUCC |
| | | | CUCCUUACG | GGUACACGUU | UUACGAGGAGACA |
| | | | AGGAGAC | AGGAAUACAU | GGAAUACAUGGUA |
| | | | (SEQ ID NO: | GGUACACGUU | CACGUU (SEQ ID |
| | | | 1476) | (SEQ ID NO: | NO: 1525) |
| | | | | 2487) | |
| R3609 | 24 | 20 | AUAGAUUGC | AGGAAUACAU | AUAGAUUGCUCCU |
| | | | UCCUUACGA | GGUACACGUU | UACGAGGAGACAG |
| | | | GGAGAC (SEQ | AGGAAUACAU | GAAUACAUGGUAC |
| | | | ID NO: 1477) | GGUACACGUU | ACGUU (SEQ ID |
| | | | | (SEQ ID NO: | NO: 1526) |
| | | | | 2487) | |
| R3610 | 23 | 20 | UAGAUUGCU | AGGAAUACAU | UAGAUUGCUCCUU |
| | | | CCUUACGAG | GGUACACGUU | ACGAGGAGACAGG |
| | | | GAGAC (SEQ | AGGAAUACAU | AAUACAUGGUACA |
| | | | ID NO: 1478) | GGUACACGUU | CGUU (SEQ ID |
| | | | | (SEQ ID NO: | NO: 1527) |
| | | | | 2487) | |
| R3611 | 22 | 20 | AGAUUGCUC | AGGAAUACAU | AGAUUGCUCCUUA |
| | | | CUUACGAGG | GGUACACGUU | CGAGGAGACAGGA |
| | | | AGAC (SEQ ID | AGGAAUACAU | AUACAUGGUACAC |
| | | | NO: 1479) | GGUACACGUU | GUU (SEQ ID |
| | | | | (SEQ ID NO: | NO: 1528) |
| | | | | 2487) | |
| R3612 | 21 | 20 | GAUUGCUCC | AGGAAUACAU | GAUUGCUCCUUAC |
| | | | UUACGAGGA | GGUACACGUU | GAGGAGACAGGAA |
| | | | GAC (SEQ ID | AGGAAUACAU | UACAUGGUACACG |
| | | | NO: 1480) | GGUACACGUU | UU (SEQ ID NO: 1529) |
| | | | | (SEQ ID NO: | |
| | | | | 2487) | |
| R3613 | 20 | 20 | AUUGCUCCU | AGGAAUACAU | AUUGCUCCUUACG |
| | | | UACGAGGAG | GGUACACGUU | AGGAGACAGGAAU |
| | | | AC (SEQ ID | AGGAAUACAU | ACAUGGUACACGU |
| | | | NO: 1481) | GGUACACGUU | U (SEQ ID NO: 1530) |
| | | | | (SEQ ID NO: | |
| | | | | 2487) | |

TABLE AH
CasΦ.12 gRNAs targeting B2M and TRAC

| | | | Repeat | Spacer | |
|---|---|---|---|---|---|
| | | | sequence | sequence | crRNA sequence |
| Name | Target | Modification | (5′ → 3′) | (5′ → 3′) | (5′ → 3′) |
| R3150 | B2M | Unmodified, | AUUGCUC | CAGUGGGGG | AUUGCUCCUUAC |
| 20-20 | Exon 2 | 2′OMe at last | CUUACGA | UGAAUUCAG | GAGGAGACCAG |
| | | 3′ base (1me) | GGAGAC | UG (SEQ ID | UGGGGGUGAAU |
| | | 2′OMe at last | (SEQ ID NO: | NO: 1434) | UCAGUG (SEQ ID |
| | | two 3′ bases | 1433) | | NO: 1435) |
| | | (2me) | | | |
| | | 2′OMe at last | | | |
| | | three 3′ bases | | | |
| | | (3me) | | | |
| R3042 | TRAC | Unmodified, | AUUGCUC | GAGUCUCUC | AUUGCUCCUUAC |
| 20-20 | Exon 1 | 1me | CUUACGA | AGCUGGUAC | GAGGAGACGAG |
| | | 2me | GGAGAC | AC (SEQ ID | UCUCUCAGCUGG |
| | | 3me | (SEQ ID NO: | NO: 1436) | UACAC (SEQ ID |
| | | | 1433) | | NO: 1437) |
| R3150 | B2M | Unmodified, | AUUGCUC | CAGUGGGGG | AUUGCUCCUUAC |
| 20-17 | Exon 2 | 1me | CUUACGA | UGAAUUCA | GAGGAGACCAG |
| | | 2me | GGAGAC | (SEQ ID NO: | UGGGGGUGAAU |
| | | 3me | (SEQ ID NO: | 1438) | UCA (SEQ ID NO: |
| | | | 1433) | | 1439) |
| R3042 | TRAC | Unmodified, | AUUGCUC | CAGUGGGGG | AUUGCUCCUUAC |
| 20-17 | Exon 1 | 1me | CUUACGA | UGAAUUCA | GAGGAGACGAG |
| | | 2me | GGAGAC | (SEQ ID NO: | UCUCUCAGCUGG |
| | | 3me | (SEQ ID NO: | 1440) | UA (SEQ ID NO: |
| | | | 1433) | | 1441) |

In some embodiments, the guide nucleic acid comprises a spacer sequence that is the same as or differs by no more than 5 nucleotides from a spacer sequence from Tables A to H by no more than 4 nucleotides from a spacer sequence from Tables A to H, by no more than 3 nucleotides from a spacer sequence from Tables A to H, no more than 2 nucleotides from a spacer sequence from Tables A to H, or no more than 1 nucleotide from a spacer sequence from Tables A to H. A difference may be addition, deletion or substitution and where there are multiple differences, the differences may be addition, deletion and/or substitution.

In some embodiments, the guide nucleic acid comprises a sequence that is the same as or differs by no more than 5 nucleotides from a sequence from Tables I to AH by no more than 4 nucleotides from a sequence from Tables I to AH, by no more than 3 nucleotides from a sequence from Tables I to X, no more than 2 nucleotides from a sequence from Table I to AH, or no more than 1 nucleotide from a sequence from Tables I to AH. A difference may be addition, deletion or substitution and where there are multiple differences, the differences may be addition, deletion and/or substitution.

In some embodiments, the guide nucleic acid comprises a sequence that is at least 30, at least 31, at least 32, at least 33, at least 34, at least 35, at least 36, at least 37, at least 38, at least 39, at least 40, at least 41, at least 42, at least 43, at least 44, at least 45, at least 46, at least 47, at least 48, at least 49, at least 50, at least 51, at least 52, at least 53, at least 54, at least 55, at least 56 or at least 57 contiguous nucleobases of a sequence from Tables I to X, AG and AH (SEQ ID NO: 547-1404, 1433-1441, 1466-1530 or 2112-2289).

In some embodiments, the guide nucleic acid comprises a sequence that is 30, 31, 32, 33, 34, 35, 36, 37, 38, 39, 40, 41, 42, 43, 44, 45, 46, 47, 48, 49, 50, 51, 52, 53, 54, 55, 56 or 57 contiguous nucleobases of a sequence from Tables I to X, AG and AH (SEQ ID NO: 547-1404, 1433-1441, 1466-1530 or 2112-2289).

In some embodiments, the guide nucleic acid comprises a sequence that is at least 30, at least 31, at least 32, at least 33, at least 34, at least 35, at least 36 or at least 37 contiguous nucleobases of a sequence from Tables Y to AF (SEQ ID NO: 1533-1933 or 2290-2467).

In some embodiments, the guide nucleic acid comprises a sequence that is 30, 31, 32, 33, 34, 35, 36 or 37 contiguous nucleobases of a sequence from Tables Y to AF (SEQ ID NO: 1533-1933 or 2290-2467).

In some embodiments, the guide nucleic acid comprises a repeat sequence from Table 2 and a spacer sequence from Tables A to H

In the sequences provided in Tables A-AH, the base T is interchangeable with U when a guide nucleic either is or comprises ribonucleic or deoxyribonucleic nucleosides.

### Coding Sequences and Expression Vectors

In some aspects, the present disclosure provides a nucleic acid encoding a programmable CasΦ nuclease disclosed herein. In some embodiments, the nucleic acid is a vector, preferably the vector is an expression vector. Suitable expression vectors are easily identifiable for the cell type of interest. For example, an expression vector comprises a suitable promoter for transcription in the cell type of interest. An expression vector can also include other elements to support transcription, such as a Woodchuck Hepatitis Virus (WHP) Posttranscriptional regulatory Element (WPRE).

In some embodiments, a nucleic acid encoding a programmable CasΦ nuclease (e.g. within an expression vector) comprises elements suitable for expression in a eukaryotic cell. In some embodiments, the nucleic acid comprises a promoter suitable for transcription in a eukaryotic cell e.g. containing a TATA box and/or a TFIIB recognition element. The nucleic acid (e.g. within an expression vector) will typically include a promoter suitable for transcription in a eukaryotic cell upstream of the sequence encoding the programmable CasΦ nuclease, and may include a transcription terminator downstream of the sequence encoding the programmable CasΦ nuclease. The nucleic acid (e.g. within an expression vector) may also include enhancer(s) upstream and/or downstream of the sequence encoding the programmable CasΦ nuclease. A promoter may be an inducible promoter. The nucleic acid may also comprise a guide RNA. Suitable promoters are well known in the art and include the CMV promoter, EF1a promoter, intron-less EF1a short promoter, SV40 promoter, human or mouse PGK1 promoter, Ubc (ubiquitin C) promoter and mouse or human U6 promoter. Suitable mammalian promoters include the EF1a promoter, intron-less EF1a short promoter, and human U6 promoter.

In some embodiments, the vector is a viral vector. In some embodiments, the vector is a retroviral vector or a lentiviral vector. In preferred embodiments, the vector is an adeno-associated viral (AAV) vector. Several serotypes are available for AAV vectors that can be used in the compositions and methods disclosed herein, including AAV1, AAV2, AAV5, AAV6, AAV8, AAV9 and AAV DJ. In more preferred embodiments, the AAV vector is an AAV DJ vector.

A vector may be integrated into a host cell genome.

In some embodiments, a vector comprises a nucleic acid encoding a programmable CasΦ nuclease. In some embodiments, a vector comprises a nucleic acid encoding a guide nucleic acid. In some embodiments, a vector comprises a donor polynucleotide. In some embodiments, a nucleic acid encoding a programmable CasΦ nuclease, a nucleic acid encoding a guide nucleic acid and a donor polynucleotide are comprised by separate vectors. In some embodiments, a vector comprises a nucleic acid encoding a programmable CasΦ nuclease and a nucleic acid encoding a guide nucleic acid.

It is well known in the field that the large size of Cas9 nucleases makes Cas9 impractical for several applications. For example, packaging vectors into viral particles becomes more difficult as the size of the vector increases. It is therefore difficult to include other components in a viral vector that includes a nucleic acid encoding a Cas9 nuclease. Accordingly, one of the advantages of the programmable CasΦ nucleases disclosed herein arises from the smaller size of the programmable CasΦ nucleases which allows vectors comprising a nucleic acid encoding a programmable CasΦ nuclease to be easily packaged into viral particles when the vector also includes nucleic acids encoding other components, such a nucleic acid encoding a guide nucleic acid and/or donor polynucleotide. In preferred embodiments, a vector encodes a nucleic acid encoding a programmable CasΦ nuclease and a nucleic acid encoding a guide nucleic acid. In preferred embodiments, a vector encodes a nucleic acid encoding a programmable CasΦ nuclease, a nucleic acid encoding a guide nucleic acid and a donor polynucleotide. In some preferred embodiments, a vector comprises up to 1 kb donor polynucleotide, a promoter for expression of a guide nucleic acid, a nucleic acid encoding the nucleic acid, a mammalian promoter for expression of a programmable CasΦ nuclease, a nucleic acid encoding the programmable CasΦ nuclease, and a polyA signal. In alternative preferred embodiments, the donor polynucleotide is included in a nucleic acid encoding a tag, such as a fluorescent protein. In further preferred embodiments, the programmable CasΦ nuclease encoded by the vector is fused or linked to two nuclear localization signals.

In some embodiments, the expression vector comprises elements suitable for expression in a prokaryotic cell. In some embodiments, the expression vector comprises a promoter suitable for transcription in a prokaryotic cell e.g. comprising a Shine Dalgarno sequence.

In some embodiments, a CasΦ nuclease, a guide nucleic acid, or a nucleic acid encoding any combination thereof, may be inserted into a host cell by manner of electroporation, nucleofection, chemical methods, transfection, transduction, transformation, or microinjection. In some embodiments, a CasΦ nuclease, a guide nucleic acid, or a nucleic acid encoding any combination thereof, may be introduced into a cell by squeezing the cell to deform it, thereby disrupting the cell membrane and allowing the CasΦ nuclease, the guide nucleic acid, or the nucleic acid encoding any combination thereof, to pass into the cell.

In some embodiments, an Amaxa 4D nucleofector may be used to carry out nucleofection. In some embodiments, the chemical method or transfection comprises lipofectamine.

Lipid nanoparticle (LNP) delivery is one of the most clinically advanced non-viral delivery systems for gene therapy. LNPs have many properties that make them ideal candidates for delivery of nucleic acids, including ease of manufacture, low cytotoxicity and immunogenicity, high efficiency of nucleic acid encapsulation and cell transfection, multidosing capabilities and flexibility of design (Kulkarni et al., (2018) Nucleic Acid Therapeutics). In some embodiments, LNP is used to deliver a nucleic acid encoding a programmable CasΦ nuclease described herein. In some embodiments, LNP is used to deliver a nucleic acid encoding a guide nucleic acid. In some embodiments, LNP is used to deliver a nucleic acid encoding a programmable CasΦ nuclease and a guide nucleic acid. In some embodiments, the LNP has an amine group to phosphate (N/P) ratio of between 2 and 10, between 3 and 10, or between 5 and 9. In preferred embodiments, the LNP has a N/P ratio of between 5 and 9. In more preferred embodiments, the LNP has a N/P ratio of 5. In some embodiments, the LNP additional components, e.g., nucleic acids, proteins, peptides, small molecules, sugars, lipids.

In more preferred embodiments, the LNP has a N/P ratio of 4 to 5. In preferred embodiments, the LNP comprises a nucleic acid encoding a programmable CasΦ nuclease, and the LNP has an N/P ratio of 4 to 5.

### Target Nucleic Acid and Sample

A wide array of samples is compatible with the compositions and methods disclosed herein. The samples, as described herein, may be used in the methods of nicking a target nucleic acid disclosed herein. The samples, as described herein, may be used in the DETECTR assay methods disclosed herein. The samples, as described herein, are compatible with any of the programmable nucleases disclosed herein and use of said programmable nuclease in a method of detecting a target nucleic acid. The samples, as described herein, are compatible with any of the compositions comprising a programmable nuclease and a buffer. Described herein are samples that contain deoxyribonucleic acid (DNA), ribonucleic acid (RNA), or both, which can be modified or detected using a programmable nuclease of the present disclosure. As described herein, programmable nucleases are activated upon binding to a target nucleic acid of interest in a sample upon hybridization of a guide nucleic acid to the target nucleic acid. Subsequently, the activated programmable nucleases exhibit sequence-independent cleavage of a nucleic acid in a reporter. The reporter additionally includes a detectable moiety, which is released upon sequence-independent cleavage of the nucleic acid in the reporter. The detectable moiety emits a detectable signal, which can be measured by various methods (e.g., spectrophotometry, fluorescence measurements, electrochemical measurements).

Various sample types comprising a target nucleic acid of interest are consistent with the present disclosure. These samples can comprise a target nucleic acid sequence for detection. In some embodiments, the detection of the target nucleic indicates an ailment, such as a disease, cancer, or genetic disorder, or genetic information, such as for phenotyping, genotyping, or determining ancestry and are compatible with the reagents and support mediums as described herein. Generally, a sample from an individual or an animal or an environmental sample can be obtained to test for presence of a disease, cancer, genetic disorder, or any mutation of interest. A biological sample from the individual may be blood, serum, plasma, saliva, urine, mucosal sample, peritoneal sample, cerebrospinal fluid, gastric secretions, nasal secretions, sputum, pharyngeal exudates, urethral or vaginal secretions, an exudate, an effusion, or tissue. A tissue sample may be dissociated or liquified prior to application to detection system of the present disclosure. A sample from an environment may be from soil, air, or water. In some instances, the environmental sample is taken as a swab from a surface of interest or taken directly from the surface of interest. In some instances, the raw sample is applied to the detection system. In some instances, the sample is diluted with a buffer or a fluid or concentrated prior to application to the detection system or be applied neat to the detection system. Sometimes, the sample is contained in no more 20 μl. The sample, in some cases, is contained in no more than 1, 5, 10, 15, 20, 25, 30, 35 40, 45, 50, 55, 60, 65, 70, 75, 80, 90, 100, 200, 300, 400, 500 μl, or any of value from 1 μl to 500 μl, preferably from 10 μL to 200 μL, or more preferably from 50 μL to 100 μL. Sometimes, the sample is contained in more than 500 μl.

In some embodiments, the target nucleic acid is single-stranded DNA. The methods, reagents, enzymes, and kits disclosed herein may enable the direct detection of a DNA encoding a sequence of interest, in particular a single-stranded DNA encoding a sequence of interest, without transcribing the DNA into RNA, for example, by using an RNA polymerase. The compositions and methods disclosed herein may enable the detection of target nucleic acid that is an amplified nucleic acid of a nucleic acid of interest. In some embodiments, the target nucleic acid is a cDNA, genomic DNA, an amplicon of genomic DNA or a DNA amplicon of an RNA. A nucleic acid can encode a sequence from a genomic locus. In some cases, the target nucleic acid that binds to the guide nucleic acid is from 5 to 100, 5 to 90, 5 to 80, 5 to 70, 5 to 60, 5 to 50, 5 to 40, 5 to 30, 5 to 25, 5 to 20, 5 to 15, or 5 to 10 nucleotides in length. The nucleic acid can be from 10 to 90, from 20 to 80, from 30 to 70, or from 40 to 60 nucleotides in length. A nucleic acid can be 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 45, 50, 60, 70, 80, 90, or 100 nucleotides in length. The target nucleic acid can encode a sequence reverse complementary to a guide nucleic acid sequence.

In some instances, the sample is taken from single-cell eukaryotic organisms; a plant or a plant cell; an algal cell; a fungal cell; an animal cell, tissue, or organ; a cell, tissue, or organ from an invertebrate animal; a cell, tissue, fluid, or organ from a vertebrate animal such as fish, amphibian, reptile, bird, and mammal; a cell, tissue, fluid, or organ from a mammal such as a human, a non-human primate, an ungulate, a feline, a bovine, an ovine, and a caprine. In some instances, the sample is taken from nematodes, protozoans, helminths, or malarial parasites. In some cases, the sample comprises nucleic acids from a cell lysate from a eukaryotic cell, a mammalian cell, a human cell, a prokaryotic cell, or a plant cell. In some cases, the sample comprises nucleic acids expressed from a cell.

The sample described herein may comprise at least one target nucleic acid. The target nucleic acid comprises a segment that is reverse complementary to a segment of a guide nucleic acid. Often, the sample comprises the segment of the target nucleic acid and at least one nucleic acid comprising at least 50% sequence identity to a segment of the target nucleic acid. Sometimes, the at least one nucleic acid comprises a segment comprising at least 60%, 70%, 75%, 80%, 85%, 90%, 91%, 92%, 93%, 94%, 95%, 96%, 97%, 98%, or 99% sequence identity to the segment of the target nucleic acid. Often, a sample comprises the segment of the target nucleic acid and at least one nucleic acid a segment comprising less than 100% sequence identity to the target nucleic acid but no less than 50% sequence identity to the segment of the target nucleic acid. Sometimes, a sample comprises the segment of the target nucleic acid and at least one nucleic acid a segment comprising less than 100% sequence identity to the target nucleic acid but no less than 60%, 70%, 75%, 80%, 85%, 90%, 91%, 92%, 93%, 94%, 95%, 96%, 97%, 98%, or 99% sequence identity to the segment of the target nucleic acid. For example, the segment of the target nucleic acid comprises a mutation as compared to at least one nucleic acid comprising a segment comprising less than 100% sequence identity to the segment of the target nucleic acid but no less than 50% sequence identity to the segment of the target nucleic acid. Sometimes, the segment of the target nucleic acid comprises a mutation as compared to at least one nucleic acid comprising a segment comprising less than 100% sequence identity to the segment of the target nucleic acid but no less than 60%, 70%, 75%, 80%, 85%, 90%, 91%, 92%, 93%, 94%, 95%, 96%, 97%, 98%, or 99% sequence identity to the segment of the target nucleic acid. Often, the segment of the target nucleic acid comprises a mutation as compared to at least one nucleic acid comprising a segment comprising less than 100% sequence identity to the segment of the target nucleic acid but no less than 50% sequence identity to the segment of the target nucleic acid. The mutation can be a mutation of 1, 2, 3, 4, 5, 6, 7, 8, 9, 10, 11, 12, 13, 14, 15, 16, 17, 18, 19, 20 or more nucleotides. Often, the mutation is a single nucleotide mutation. The single nucleotide mutation can be a single nucleotide polymorphism (SNP), which is a single base pair variation in a DNA sequence present in less than 1% of a population. Sometimes, the target nucleic acid comprises a single nucleotide mutation, wherein the single nucleotide mutation comprises the wild type variant of the SNP. The single nucleotide mutation or SNP can be associated with a phenotype of the sample or a phenotype of the organism from which the sample was taken. The SNP, in some cases, is associated with altered phenotype from wild type phenotype. Often, the segment of the target nucleic acid sequence comprises a deletion as compared to at least one nucleic acid comprising a segment comprising less than 100% sequence identity to the segment of the target nucleic acid but no less than 50% sequence identity to the segment of the target nucleic acid. The mutation can be a deletion of 1, 2, 3, 4, 5, 6, 7, 8, 9, 10, 11, 12, 13, 14, 15, 16, 17, 18, 19, 20 or more nucleotides. The mutation can be a deletion of about 5, about 10, about 15, about 20, about 25, about 30, about 35, about 40, about 45, about 50, about 55, about 60, about 65, about 70, about 75, about 80, about 85, about 90, about 95, about 100, about 200, about 300, about 400, about 500, about 600, about 700, about 800, about 900, or about 1000 nucleotides. The mutation can be a deletion of from 1 to 5, from 5 to 10, from 10 to 15, from 15 to 20, from 20 to 25, from 25 to 30, from 30 to 35, from 35 to 40, from 40 to 45, from 45 to 50, from 50 to 55, from 55 to 60, from 60 to 65, from 65 to 70, from 70 to 75, from 75 to 80, from 80 to 85, from 85 to 90, from 90 to 95, from 95 to 100, from 100 to 200, from 200 to 300, from 300 to 400, from 400 to 500, from 500 to 600, from 600 to 700, from 700 to 800, from 800 to 900, from 900 to 1000, from 1 to 50, from 1 to 100, from 25 to 50, from 25 to 100, from 50 to 100, from 100 to 500, from 100 to 1000, or from 500 to 1000 nucleotides. The segment of the target nucleic acid that the guide nucleic acid of the methods describe herein binds to comprises the mutation, such as the SNP or the deletion. The mutation can be a single nucleotide mutation or a SNP. The SNP can be a synonymous substitution or a nonsynonymous substitution. The nonsynonymous substitution can be a missense substitution or a nonsense point mutation. The synonymous substitution can be a silent substitution. The mutation can be a deletion of one or more nucleotides. Often, the single nucleotide mutation, SNP, or deletion is associated with a disease such as cancer or a genetic disorder. The mutation, such as a single nucleotide mutation, a SNP, or a deletion, can be encoded in the sequence of a target nucleic acid from the germline of an organism or can be encoded in a target nucleic acid from a diseased cell, such as a cancer cell.

The sample used for disease testing may comprise at least one target nucleic acid that can bind to a guide nucleic acid of the reagents described herein. The sample used for disease testing may comprise at least nucleic acid of interest that is amplified to produce a target nucleic acid that can bind to a guide nucleic acid of the reagents described herein. The nucleic acid of interest can comprise DNA, RNA, or a combination thereof.

The target nucleic acid (e.g., a target DNA) may be a portion of a nucleic acid from a virus or a bacterium or other agents responsible for a disease in the sample. The target nucleic acid may be a portion of a nucleic acid from a gene expressed in a cancer or genetic disorder in the sample. In some cases, the sequence is a segment of a target nucleic acid sequence. A segment of a target nucleic acid sequence can be from a genomic locus, a transcribed mRNA, or a reverse transcribed cDNA. A segment of a target nucleic acid sequence can be from 5 to 100, 5 to 90, 5 to 80, 5 to 70, 5 to 60, 5 to 50, 5 to 40, 5 to 30, 5 to 25, 5 to 20, 5 to 15, or 5 to 10 nucleotides in length. A segment of a target nucleic acid sequence can be 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 45, 50, 60, 70, 80, 90, or 100 nucleotides in length. The sequence of the target nucleic acid segment can be reverse complementary to a segment of a guide nucleic acid sequence. The target nucleic acid may comprise a genetic variation (e.g., a single nucleotide polymorphism), with respect to a standard sample, associated with a disease phenotype or disease predisposition. The target nucleic acid may be an amplicon of a portion of an RNA, may be a DNA, or may be a DNA amplicon from any organism in the sample.

In some embodiments, the target nucleic acid sequence comprises a nucleic acid sequence of a virus or a bacterium or other agents responsible for a disease in the sample. In some embodiments, the target nucleic acid comprises DNA that is reverse transcribed from RNA using a reverse transcriptase prior to detection by a programmable nuclease using the compositions, systems, and methods disclosed herein. The target nucleic acid, in some cases, is a portion of a nucleic acid from a sexually transmitted infection or a contagious disease, in the sample. In some cases, the target nucleic acid is a portion of a nucleic acid from a genomic locus, or any DNA amplicon, such as a reverse transcribed mRNA or a cDNA from a gene locus, a transcribed mRNA, or a reverse transcribed cDNA from a gene locus in at least one of: human immunodeficiency virus (HIV), human papillomavirus (HPV), chlamydia, gonorrhea, syphilis, trichomoniasis, sexually transmitted infection, malaria, Dengue fever, Ebola, chikungunya, and leishmaniasis. Pathogens include viruses, fungi, helminths, protozoa, malarial parasites, Plasmodium parasites, Toxoplasma parasites, and Schistosoma parasites. Helminths include roundworms, heartworms, and phytophagous nematodes, flukes, Acanthocephala, and tapeworms. Protozoan infections include infections from Giardia spp., Trichomonas spp., African trypanosomiasis, amoebic dysentery, babesiosis, balantidial dysentery, Chaga's disease, coccidiosis, malaria and toxoplasmosis. Examples of pathogens such as parasitic/protozoan pathogens include, but are not limited to: Plasmodium falciparum, P. vivax, Trypanosoma cruzi and Toxoplasma gondii. Fungal pathogens include, but are not limited to Cryptococcus neoformans, Histoplasma capsulatum, Coccidioides immitis, Blastomyces dermatitidis, Chlamydia trachomatis, and Candida albicans. Pathogenic viruses include but are not limited to coronavirus; immunodeficiency virus (e.g., HIV); influenza virus; dengue; West Nile virus; herpes virus; yellow fever virus; Hepatitis Virus C; Hepatitis Virus A; Hepatitis Virus B; papillomavirus; and the like. Pathogens include, e.g., HIV virus, Mycobacterium tuberculosis, Streptococcus agalactiae, methicillin-resistant Staphylococcus aureus, Legionella pneumophila, Streptococcus pyogenes, Escherichia coli, Neisseria gonorrhoeae, Neisseria meningitidis, Pneumococcus, Cryptococcus neoformans, Histoplasma capsulatum, Hemophilus influenzae B, Treponema pallidum, Lyme disease spirochetes, Pseudomonas aeruginosa, Mycobacterium leprae, Brucella abortus, rabies virus, influenza virus, cytomegalovirus, herpes simplex virus I, herpes simplex virus II, human serum parvo-like virus, respiratory syncytial virus (RSV), M. genitalium, T. vaginalis, varicella-zoster virus, hepatitis B virus, hepatitis C virus, measles virus, adenovirus, human T-cell leukemia viruses, Epstein-Barr virus, murine leukemia virus, mumps virus, vesicular stomatitis virus, Sindbis virus, lymphocytic choriomeningitis virus, wart virus, blue tongue virus, Sendai virus, feline leukemia virus, Reovirus, polio virus, simian virus 40, mouse mammary tumor virus, dengue virus, rubella virus, West Nile virus, Plasmodium falciparum, Plasmodium vivax, Toxoplasma gondii, Trypanosoma rangeli, Trypanosoma cruzi, Trypanosoma rhodesiense, Trypanosoma brucei, Schistosoma mansoni, Schistosoma japonicum, Babesia bovis, Eimeria tenella, Onchocerca volvulus, Leishmania tropica, Mycobacterium tuberculosis, Trichinella spiralis, Theileria parva, Taenia hydatigena, Taenia ovis, Taenia saginata, Echinococcus granulosus, Mesocestoides corti, Mycoplasma arthritidis, M. hyorhinis, M orale, M. arginini, Acholeplasma laidlawii, M. salivarium and M. pneumoniae. In some cases, the target sequence is a portion of a nucleic acid from a genomic locus, a transcribed mRNA, or a reverse transcribed cDNA from a gene locus of bacterium or other agents responsible for a disease in the sample comprising a mutation that confers resistance to a treatment, such as a single nucleotide mutation that confers resistance to antibiotic treatment. In some cases, the mutation that confers resistance to a treatment is a deletion.

Compositions and methods of the disclosure can be used for cell line engineering (e.g., engineering a cell from a cell line for bioproduction). For example, compositions and methods of the disclosure can be used to express a desired protein from a cell line. In some embodiments, the target nucleic acid sequence comprises a nucleic acid sequence of a cell line. In some embodiments, the target nucleic acid sequence comprises a genomic nucleic acid sequence of a cell line. In some embodiments, the cell line is a Chinese hamster ovary cell line (CHO), human embryonic kidney cell line (HEK), cell lines derived from cancer cells, cell lines derived from lymphocytes, and the like. Non-limiting examples of cell lines includes: C8161, CCRF-CEM, MOLT, mIMCD-3, NHDF, HeLa-S3, Huh1, Huh4, Huh7, HUVEC, HASMC, HEKn, HEKa, MiaPaCell, Panc1, PC-3, TF1, CTLL-2, CIR, Rat6, CV1, RPTE, A10, T24, J82, A375, ARH-77, Calu1, SW480, SW620, SKOV3, SK-UT, CaCo2, P388D1, SEM-K2, WEHI-231, HB56, TIB55, Jurkat, J45.01, LRMB, Bcl-1, BC-3, IC21, DLD2, Raw264.7, NRK, NRK-52E, MRC5, MEF, Hep G2, HeLa B, HeLa T4, COS, COS-1, COS-6, COS-M6A, BS-C-1 monkey kidney epithelial, BALB/3T3 mouse embryo fibroblast, 3T3 Swiss, 3T3-L1, 132-d5 human fetal fibroblasts; 10.1 mouse fibroblasts, 293-T, 3T3, 721, 9L, A2780, A2780ADR, A2780cis, A172, A20, A253, A431, A-549, ALC, AsPC-1, B16, B35, BCP-1 cells, BEAS-2B, bEnd.3, BHK-21, BR 293, BxPC3, C3H-10T1/2, C6/36, Cal-27, Capan-1, CHO, CHO-7, CHO-IR, CHO-K1, CHO-K2, CHO-S, CHO-T, CHO Dhfr −/−. COR-L23, COR-L23/CPR, COR-L23/5010, COR-L23/R23, COS-7, COV-434, CML T1, CMT, CT26, D17, DH82, DU145, DuCaP, EL4, EM2, EM3, EMT6/AR1, EMT6/AR10.0, FM3, H1299, H69, HAP1, HB54, HB55, HCA2, HEK-293, HeLa, Hepa1-6, Hep3B, Hepa1c1c7, HL-60, HMEC, HT-29, Jurkat, JY cells, K562 cells, Ku812, KCL22, KG1, KYO1, LNCap, Ma-Mel 1-48, MC-38, MCF-7, MCF-10A, MDA-MB-231, MDA-MB-468, MDA-MB-435, MDCK II, MDCK II, MOR/0.2R, MONO-MAC 6, MTD-1A, MyEnd, NCI-H69/CPR, NCI-H69/LX10, NCI-H69/LX20, NCI-H69/LX4, NIH-3T3, NALM-1, Neuro2A, NK92, NW-145, OPCN/OPCT cell lines, Peer, PNT-1A/PNT 2, RenCa, RIN-5F, RMA/RMAS, Saos-2 cells, Sf-9, SkBr3, T2, T-47D, T84, THP1 cell line, U373, U87, U937, VCaP, Vero cells, WM39, WT-49, X63, YAC-1, and YAR. Non-limiting examples of other cells that can be used with the disclosure include immune cells, such as CART, T-cells, B-cells, NK cells (including iNK cells), granulocytes, basophils, eosinophils, neutrophils, mast cells, monocytes, macrophages, dendritic cells, antigen-presenting cells (APC), or adaptive cells. Non-limiting examples of cells that can be used with this disclosure also include plant cells, such as parenchyma, sclerenchyma, collenchyma, xylem, phloem, germline (e.g., pollen). Cells may be from lycophytes, ferns, gymnosperms, angiosperms, bryophytes, charophytes, chloropytes, rhodophytes, or glaucophytes. Cells may be obtained from non-human animals, including, but not limited to, rats, dogs, rabbits, cats, and monkeys. Non-limiting examples of cells that can be used with this disclosure also include stem cells, such as human stem cells, animal stem cells, stem cells that are not derived from human embryonic stem cells, embryonic stem cells, mesenchymal stem cells, pluripotent stem cells, induced pluripotent stem cells (iPS), somatic stem cells, adult stem cells, hematopoietic stem cells, tissue-specific stem cells. Non-limiting examples of cells that can be used with this disclosure also include neuronal cells from various organs of an animal, e.g., brain, heart, lung, liver, pancreas, and muscle. In preferred embodiments, the cells that can be used with the disclosure are T cells, such as CAR-T (CART) cells.

CHO cells are an epithelial cell line which is particularly useful in biological and medical research. In particular, CHO cells are frequently used for the industrial production of recombinant therapeutics. In some embodiments, a CasΦ polypeptide disclosed herein is expressed in a CHO cell. In some embodiments, a CasΦ polypeptide disclosed herein complexed with a guide nucleic is expressed in a CHO cell. In some embodiments, a method disclosed herein comprises modifying or editing a CHO cell. In some embodiments, a modified CHO cell is provided wherein the CHO cell is modified by a CasΦ polypeptide disclosed herein. In some embodiments, a CHO cell is provided wherein the CHO cell comprises a CasΦ polypeptide disclosed herein.

T cells are important therapeutic targets. In some embodiments, a CasΦ polypeptide disclosed herein is expressed in a T cell. In some embodiments, a CasΦ polypeptide disclosed herein complexed with a guide nucleic is expressed in a T cell. In some embodiments, a method disclosed herein comprises modifying or editing a T cell. In some embodiments, a method disclosed herein comprises modifying a PDCD1 gene of a T cell. In some embodiments, a method disclosed herein comprises modifying a TRAC gene of a T cell. In some embodiments, a method disclosed herein comprises modifying a B2M gene of a T cell. In some embodiments, a method disclosed herein comprises modifying a PDCD1 gene of a T cell, a TRAC gene of a T cell, a B2M gene of a T cell or a combination thereof. In some embodiments, a method disclosed herein comprises modifying a PDCD1 gene, a TRAC gene, and a B2M gene of a T cell. In some embodiments, a modified T cell is provided wherein the T cell is modified by a CasΦ polypeptide disclosed herein. In some embodiments, a T cell is provided wherein the T cell comprises a CasΦ polypeptide disclosed herein.

T cells, also known as T lymphocytes, are easily identifiable by the surface expression of the T-cell receptor (TCR). In some embodiments, the T cells include one or more subsets of T cells, such as CD4+ cells, CD8+ cells, and sub-populations thereof. In some embodiments, a T cell is a CD4+ cell. In some embodiments, a T cell is a CD8+ T cells. In some embodiments, a population of T cells comprises CD4+ T cells and CD8+ T cells. In some embodiments, T cells comprise TCR-T, Tscm, or iT cells.

Sub-populations of CD4+ and CD8+ T cells include naive T cells, effector T cells, memory T cells, immature T cells, mature T cells, helper T cells, cytotoxic T cells, regulatory T cells, alpha/beta T cells, and delta/gamma T cells. Sub-types of memory T cells include stem cell memory T cells, central memory T cells, effector memory T cells, and terminally differentiated effector memory T cells. Sub-types of helper T cells, include T helper 1 cells, T helper 2 cells, T helper 3 cells, T helper 17 cells, T helper 9 cells, T helper 22 cells, and follicular helper T cells. In some embodiments, the cell is a regulatory T cell (Treg).

CART cells are T cells that have been genetically engineered to express unique chimeric antigen receptors (CARs) targeting specific antigens. CART cells are important targets for immunotherapy. In some embodiments, a CasΦ polypeptide disclosed herein is expressed in a CART cell. In some embodiments, a CasΦ polypeptide disclosed herein complexed with a guide nucleic is expressed in a CART cell. In some embodiments, a method disclosed herein comprises modifying or editing a CART cell. In some embodiments, a modified CART cell is provided wherein the CART cell is modified by a CasΦ polypeptide disclosed herein. In some embodiments, a CART cell is provided wherein the CART cell comprises a CasΦ polypeptide disclosed herein.

Modified stem cells and methods of modifying stem cells are also provided. In some embodiments, a CasΦ polypeptide disclosed herein is expressed in a stem cell. In some embodiments, a CasΦ polypeptide disclosed herein complexed with a guide nucleic is expressed in a stem cell. In some embodiments, a method disclosed herein comprises modifying or editing a stem cell. In some embodiments, a modified stem cell is provided wherein a stem cell is modified by a CasΦ polypeptide disclosed herein. In some embodiments, a stem cell is provided wherein the stem cell comprises a CasΦ polypeptide disclosed herein. In some embodiments, a modified stem cell is obtained or is obtainable by a method disclosed herein. In some embodiments, a modified stem cell is provided wherein the CART cell is modified by a CasΦ polypeptide disclosed herein.

Induced pluripotent stem cells (iPSCs) are pluripotent stem cells that are generated from somatic cells. They can propagate indefinitely and give rise to any cell type in the body. These features make iPSCs a powerful tool for researching human disease and provide a promising prospect for cell therapies for a range of medical conditions. iPSCs can be generated in a patient-specific manner and used in autologous transplant, thereby overcoming complications of rejection by the host immune system (Moradi et al. (2019), Stem Cell Research & Therapy).

In some embodiments, a CasΦ polypeptide disclosed herein is expressed in an induced pluripotent stem cell. In some embodiments, a CasΦ polypeptide disclosed herein complexed with a guide nucleic is expressed in an induced pluripotent stem cell. In some embodiments, a method disclosed herein comprises modifying or editing an induced pluripotent stem cell. In some embodiments, a modified induced pluripotent stem cell is provided wherein an induced pluripotent stem cell is modified by a CasΦ polypeptide disclosed herein. In some embodiments, an induced pluripotent stem cell is provided wherein the induced pluripotent stem cell comprises a CasΦ polypeptide disclosed herein. In some embodiments, a modified induced pluripotent cell is obtained or is obtainable by a method disclosed herein.

Hematopoietic stem cells (HSCs) are identifiable by the marker CD34. HSCs are stem cells that differentiate to give rise blood cells, such as T and B lymphocytes, erythrocytes, monocytes and macrophages. HSCs are important cells for future stem cell therapies as they have the potential to be used to treat genetic blood cell diseases (Morgan et al. (2017), Cell Stem Cell).

In some embodiments, a CasΦ polypeptide disclosed herein is expressed in a hematopoietic stem cell. In some embodiments, a CasΦ polypeptide disclosed herein complexed with a guide nucleic is expressed in a hematopoietic stem cell. In some embodiments, a method disclosed herein comprises modifying or editing a hematopoietic stem cell. In some embodiments, a modified hematopoietic stem cell is provided wherein a hematopoietic stem cell is modified by a CasΦ polypeptide disclosed herein. In some embodiments, a hematopoietic stem cell is provided wherein the hematopoietic stem cell comprises a CasΦ polypeptide disclosed herein. In some embodiments, a modified hematopoietic stem cell is obtained or is obtainable by a method disclosed herein.

Compositions and methods of the disclosure can be used for agricultural engineering. For example, compositions and methods of the disclosure can be used to confer desired traits on a plant. A plant can be engineered for the desired physiological and agronomic characteristic using the present disclosure. In some embodiments, the target nucleic acid sequence comprises a nucleic acid sequence of a plant. In some embodiments, the target nucleic acid sequence comprises a genomic nucleic acid sequence of a plant cell. In some embodiments, the target nucleic acid sequence comprises a nucleic acid sequence of an organelle of a plant cell. In some embodiments, the target nucleic acid sequence comprises a nucleic acid sequence of a chloroplast of a plant cell.

The plant can be a monocotyledonous plant. The plant can be a dicotyledonous plant. Non-limiting examples of orders of dicotyledonous plants include Magniolales, Illiciales, Laurales, Piperales, Aristochiales, Nymphaeales, Ranunculales, Papeverales, Sarraceniaceae, Trochodendrales, Hamamelidales, Eucomiales, Leitneriales, Myricales, Fagales, Casuarinales, Caryophyllales, Batales, Polygonales, Plumbaginales, Dilleniales, Theales, Malvales, Urticales, Lecythidales, Violales, Salicales, Capparales, Ericales, Diapensales, Ebenales, Primulales, Rosales, Fabales, Podostemales, Haloragales, Myrtales, Cornales, Proteales, San tales, Rafflesiales, Celastrales, Euphorbiales, Rhamnales, Sapindales, Juglandales, Geraniales, Polygalales, Umbellales, Gentianales, Polemoniales, Lamiales, Plantaginales, Scrophulariales, Campanulales, Rubiales, Dipsacales, and Asterales.

Non-limiting examples of orders of monocotyledonous plants include Alismatales, Hydrocharitales, Najadales, Triuridales, Commelinales, Eriocaulales, Restionales, Poales, Juncales, Cyperales, Typhales, Bromeliales, Zingiberales, Arecales, Cyclanthales, Pandanales, Arales, Lilliales, and Orchid ales. A plant can belong to the order, for example, Gymnospermae, Pinales, Ginkgoales, Cycadales, Araucariales, Cupressales and Gnetales.

Non-limiting examples of plants include plant crops, fruits, vegetables, grains, soy bean, corn, maize, wheat, seeds, tomatoes, rice, cassava, sugarcane, pumpkin, hay, potatoes, cotton, cannabis, tobacco, flowering plants, conifers, gymnosperms, ferns, clubmosses, hornworts, liverworts, mosses, wheat, maize, rice, millet, barley, tomato, apple, pear, strawberry, orange, acacia, carrot, potato, sugar beets, yam, lettuce, spinach, sunflower, rape seed, Arabidopsis, alfalfa, amaranth, apple, apricot, artichoke, ash tree, asparagus, avocado, banana, barley, beans, beet, birch, beech, blackberry, blueberry, broccoli, Brussel's sprouts, cabbage, canola, cantaloupe, carrot, cassava, cauliflower, cedar, a cereal, celery, chestnut, cherry, Chinese cabbage, citrus, clementine, clover, coffee, corn, cotton, cowpea, cucumber, cypress, eggplant, elm, endive, eucalyptus, fennel, figs, fir, geranium, grape, grapefruit, groundnuts, ground cherry, gum hemlock, hickory, kale, kiwifruit, kohlrabi, larch, lettuce, leek, lemon, lime, locust, pine, maidenhair, maize, mango, maple, melon, millet, mushroom, mustard, nuts, oak, oats, oil palm, okra, onion, orange, an ornamental plant or flower or tree, papaya, palm, parsley, parsnip, pea, peach, peanut, pear, peat, pepper, persimmon, pigeon pea, pine, pineapple, plantain, plum, pomegranate, potato, pumpkin, radicchio, radish, rapeseed, raspberry, rice, rye, sorghum, safflower, sallow, soybean, spinach, spruce, squash, strawberry, sugar beet, sugarcane, sunflower, sweet potato, sweet corn, tangerine, tea, tobacco, tomato, trees, triticale, turf grasses, turnips, vine, walnut, watercress, watermelon, wheat, yams, yew, and zucchini. A plant can include algae.

In some embodiments, the target nucleic acid sequence comprises a nucleic acid sequence of a virus, a bacterium, or other pathogen responsible for a disease in a plant (e.g., a crop). Methods and compositions of the disclosure can be used to treat or detect a disease in a plant. For example, the methods of the disclosure can be used to target a viral nucleic acid sequence in a plant. A programmable nuclease of the disclosure (e.g., CasΦ) can cleave the viral nucleic acid. In some embodiments, the target nucleic acid sequence comprises a nucleic acid sequence of a virus or a bacterium or other agents (e.g., any pathogen) responsible for a disease in the plant (e.g., a crop). In some embodiments, the target nucleic acid comprises DNA that is reverse transcribed from RNA using a reverse transcriptase prior to detection by a programmable nuclease using the compositions, systems, and methods disclosed herein. The target nucleic acid, in some cases, is a portion of a nucleic acid from a virus or a bacterium or other agents responsible for a disease in the plant (e.g., a crop). In some cases, the target nucleic acid is a portion of a nucleic acid from a genomic locus, or any DNA amplicon, such as a reverse transcribed mRNA or a cDNA from a gene locus, a transcribed mRNA, or a reverse transcribed cDNA from a gene locus in at a virus or a bacterium or other agents (e.g., any pathogen) responsible for a disease in the plant (e.g., a crop). A virus infecting the plant can be an RNA virus. A virus infecting the plant can be a DNA virus. Non-limiting examples of viruses that can be targeted with the disclosure include Tobacco mosaic virus (TMV), Tomato spotted wilt virus (TSWV), Cucumber mosaic virus (CMV), Potato virus Y (PVY), Cauliflower mosaic virus (CaMV) (RT virus), Plum pox virus (PPV), Brome mosaic virus (BMV) and Potato virus X (PVX).

The sample used for cancer testing may comprise at least one target nucleic acid that can bind to a guide nucleic acid of the reagents described herein. The target nucleic acid, in some cases, comprises a portion of a gene comprising a mutation associated with cancer, a gene whose overexpression is associated with cancer, a tumor suppressor gene, an oncogene, a checkpoint inhibitor gene, a gene associated with cellular growth, a gene associated with cellular metabolism, or a gene associated with cell cycle. Sometimes, the target nucleic acid encodes a cancer biomarker, such as a prostate cancer biomarker or non-small cell lung cancer. In some cases, the assay can be used to detect “hotspots” in target nucleic acids that can be predictive of lung cancer. In some cases, the target nucleic acid comprises a portion of a nucleic acid that is associated with a blood fever. In some cases, the target nucleic acid is a portion of a nucleic acid from a genomic locus, any DNA amplicon of, a reverse transcribed mRNA, or a cDNA from a locus of at least one of: ALK, APC, ATM, AXIN2, BAP1, BARD1, BLM, BMPR1A, BRCA1, BRCA2, BRIP1, CASR, CDC73, CDH1, CDK4, CDKN1B, CDKN1C, CDKN2A, CEBPA, CHEK2, CTNNA1, DICER1, DIS3L2, EGFR, EPCAM, FH, FLCN, GATA2, GPC3, GREM1, HOXB13, HRAS, KIT, MAX, MEN1, MET, MITF, MLH1, MSH2, MSH3, MSH6, MUTYH, NBN, NF1, NF2, NTHL1, PALB2, PDGFRA, PHOX2B, PMS2, POLD1, POLE, POT1, PRKAR1A, PTCH1, PTEN, RAD50, RAD51C, RAD51D, RB1, RECQL4, RET, RUNX1, SDHA, SDHAF2, SDHB, SDHC, SDHD, SMAD4, SMARCA4, SMARCB1, SMARCE1, STK11, SUFU, TERC, TERT, TMEM127, TP53, TSC1, TSC2, VHL, WRN, and WT1. Any region of the aforementioned gene loci can be probed for a mutation or deletion using the compositions and methods disclosed herein. For example, in the EGFR gene locus, the compositions and methods for detection disclosed herein can be used to detect a single nucleotide polymorphism or a deletion. The SNP or deletion can occur in a non-coding region or a coding region. The SNP or deletion can occur in an Exon, such as Exon19. A SNP, deletion, or other mutation may mediate gene knockout.

The sample used for genetic disorder testing may comprise at least one target nucleic acid that can bind to a guide nucleic acid of the reagents described herein. In some embodiments, the genetic disorder is hemophilia, sickle cell anemia, 0-thalassemia, Duchene muscular dystrophy, severe combined immunodeficiency, Huntington's disease, or cystic fibrosis. The target nucleic acid, in some cases, is from a gene with a mutation associated with a genetic disorder, from a gene whose overexpression is associated with a genetic disorder, from a gene associated with abnormal cellular growth resulting in a genetic disorder, or from a gene associated with abnormal cellular metabolism resulting in a genetic disorder. In some cases, the target nucleic acid is a nucleic acid from a genomic locus, a transcribed mRNA, or a reverse transcribed mRNA, a DNA amplicon of or a cDNA from a locus of at least one of: CFTR, FMR1, SMN1, ABCB11, ABCC8, ABCD1, ACAD9, ACADM, ACADVL, ACAT1, ACOX1, ACSF3, ADA, ADAMTS2, ADGRG1, AGA, AGL, AGPS, AGXT, AIRE, ALDH3A2, ALDOB, ALG6, ALMS1, ALPL, AMT, AQP2, ARG1, ARSA, ARSB, ASL, ASNS, ASPA, ASS1, ATM, ATP6V1B1, ATP7A, ATP7B, ATRX, BBS1, BBS10, BBS12, BBS2, BCKDHA, BCKDHB, BCS1L, BLM, BSND, CAPN3, CBS, CDH23, CEP290, CERKL, CHM, CHRNE, CIITA, CLN3, CLN5, CLN6, CLN8, CLRN1, CNGB3, COL27A1, COL4A3, COL4A4, COL4A5, COL7A1, CPS1, CPT1A, CPT2, CRB1, CTNS, CTSK, CYBA, CYBB, CYP11B1, CYP11B2, CYP17A1, CYP19A1, CYP27A1, DBT, DCLRElC, DHCR7, DHDDS, DLD, DMD, DNAH5, DNAI1, DNAI2, DYSF, EDA, EIF2B5, EMD, ERCC6, ERCC8, ESCO2, ETFA, ETFDH, ETHEl, EVC, EVC2, EYS, F9, FAH, FAM161A, FANCA, FANCC, FANCG, FH, FKRP, FKTN, G6PC, GAA, GALC, GALK1, GALT, GAMT, GBA, GBE1, GCDH, GFM1, GJB1, GJB2, GLA, GLB1, GLDC, GLE1, GNE, GNPTAB, GNPTG, GNS, GRHPR, HADHA, HAX1, HBA1, HBA2, HBB, HEXA, HEXB, HGSNAT, HLCS, HMGCL, HOGA1, HPS1, HPS3, HSD17B4, HSD3B2, HYAL1, HYLS1, IDS, IDUA, IKBKAP, IL2RG, IVD, KCNJ11, LAMA2, LAMA3, LAMB3, LAMC2, LCA5, LDLR, LDLRAP1, LHX3, LIFR, LIPA, LOXHD1, LPL, LRPPRC, MAN2B1, MCOLN1, MED17, MESP2, MFSD8, MKS1, MLC1, MMAA, MMAB, MMACHC, MMADHC, MPI, MPL, MPV17, MTHFR, MTM1, MTRR, MTTP, MUT, MYO7A, NAGLU, NAGS, NBN, NDRG1, NDUFAF5, NDUFS6, NEB, NPC1, NPC2, NPHS1, NPHS2, NR2E3, NTRK1, OAT, OPA3, OTC, PAH, PC, PCCA, PCCB, PCDH15, PDHA1, PDHB, PEX1, PEX10, PEX12, PEX2, PEX6, PEX7, PFKM, PHGDH, PKHD1, PMM2, POMGNT1, PPT1, PROP1, PRPS1, PSAP, PTS, PUS1, PYGM, RAB23, RAG2, RAPSN, RARS2, RDH12, RMRP, RPE65, RPGRIP1L, RS1, RTEL1, SACS, SAMID1, SEPSECS, SGCA, SGCB, SGCG, SGSH, SLC12A3, SLC12A6, SLC17A5, SLC22A5, SLC25A13, SLC25A15, SLC26A2, SLC26A4, SLC35A3, SLC37A4, SLC39A4, SLC4A11, SLC6A8, SLC7A7, SMARCAL1, SMPD1, STAR, SUMF1, TAT, TCIRG1, TECPR2, TFR2, TGM1, TH, TMEM216, TPP1, TRMU, TSFM, TTPA, TYMP, USH1C, USH2A, VPS13A, VPS13B, VPS45, VRK1, VSX2, WNT10A, XPA, XPC, and ZFYVE26.

The sample used for phenotyping testing may comprise at least one target nucleic acid that can bind to a guide nucleic acid of the reagents described herein. The target nucleic acid, in some cases, is a nucleic acid encoding a sequence associated with a phenotypic trait.

The sample used for genotyping testing may comprise at least one target nucleic acid that can bind to a guide nucleic acid of the reagents described herein. The target nucleic acid, in some cases, is a nucleic acid encoding a sequence associated with a genotype of interest.

The sample used for ancestral testing may comprise at least one target nucleic acid that can bind to a guide nucleic acid of the reagents described herein. The target nucleic acid, in some cases, is a nucleic acid encoding a sequence associated with a geographic region of origin or ethnic group.

The sample can be used for identifying a disease status. For example, a sample is any sample described herein, and is obtained from a subject for use in identifying a disease status of a subject. The disease can be a cancer or genetic disorder. Sometimes, a method comprises obtaining a serum sample from a subject; and identifying a disease status of the subject. Often, the disease status is prostate disease status, but the status of any disease can be assessed.

In some instances, the target nucleic acid is a single stranded nucleic acid. Alternatively, or in combination, the target nucleic acid is a double stranded nucleic acid and is prepared into single stranded nucleic acids before or upon contacting the reagents. The target nucleic acid may be a reverse transcribed RNA, DNA, DNA amplicon, synthetic nucleic acids, or nucleic acids found in biological or environmental samples. The target nucleic acids include but are not limited to mRNA, rRNA, tRNA, non-coding RNA, long non-coding RNA, and microRNA (miRNA). In some cases, the target nucleic acid is single-stranded DNA (ssDNA) or mRNA. In some cases, the target nucleic acid is from a virus, a parasite, or a bacterium described herein. In some cases, the target nucleic acid is transcribed from a gene as described herein and then reverse transcribed into a DNA amplicon. In some cases, miRNA is extracted using a mirVANA kit. In some cases, RNA may be treated with shrimp alkaline phosphatase to remove phosphates from the 5′ and 3′ ends of an RNA for analysis. RNA analysis may further comprise the use of a thermocycler, SR Adaptors for Illumina, ligation enzymes, reverse transcriptase, and suitable primers for polymerase chain reaction.

A number of target nucleic acids are consistent with the methods and compositions disclosed herein. Some methods described herein can detect a target nucleic acid present in the sample in various concentrations or amounts as a target nucleic acid population. In some cases, the sample has at least 2 target nucleic acids. In some cases, the sample has at least 3, 5, 10, 20, 30, 40, 50, 100, 200, 300, 400, 500, 600, 700, 800, 900, 1000, 2000, 3000, 4000, 5000, 6000, 7000, 8000, 9000, or 10000 target nucleic acids. In some cases, the sample as from 1 to 10,000, from 100 to 8000, from 400 to 6000, from 500 to 5000, from 1000 to 4000, or from 2000 to 3000 target nucleic acids. In some cases, the method detects target nucleic acid present at least at one copy per 10 non-target nucleic acids, 10² non-target nucleic acids, 10³ non-target nucleic acids, 10⁴ non-target nucleic acids, 10⁵ non-target nucleic acids, 10⁶ non-target nucleic acids, 10⁷ non-target nucleic acids, 10⁸ non-target nucleic acids, 10⁹ non-target nucleic acids, or 10¹⁰ non-target nucleic acids. Often, the target nucleic acid can be from 0.05% to 20% of total nucleic acids in the sample. Sometimes, the target nucleic acid is from 0.1% to 10% of the total nucleic acids in the sample. The target nucleic acid, in some cases, is from 0.1% to 5% of the total nucleic acids in the sample. The target nucleic acid can also be from 0.1% to 1% of the total nucleic acids in the sample. The target nucleic acid can be DNA or RNA. The target nucleic acid can be any amount less than 100% of the total nucleic acids in the sample. The target nucleic acid can be 100% of the total nucleic acids in the sample.

In some embodiments, the sample comprises a target nucleic acid at a concentration of less than 1 nM, less than 2 nM, less than 3 nM, less than 4 nM, less than 5 nM, less than 6 nM, less than 7 nM, less than 8 nM, less than 9 nM, less than 10 nM, less than 20 nM, less than 30 nM, less than 40 nM, less than 50 nM, less than 60 nM, less than 70 nM, less than 80 nM, less than 90 nM, less than 100 nM, less than 200 nM, less than 300 nM, less than 400 nM, less than 500 nM, less than 600 nM, less than 700 nM, less than 800 nM, less than 900 nM, less than 1 μM, less than 2 μM, less than 3 μM, less than 4 μM, less than 5 μM, less than 6 μM, less than 7 μM, less than 8 μM, less than 9 μM, less than 10 μM, less than 100 μM, or less than 1 mM. In some embodiments, the sample comprises a target nucleic acid sequence at a concentration of from 1 nM to 2 nM, from 2 nM to 3 nM, from 3 nM to 4 nM, from 4 nM to 5 nM, from 5 nM to 6 nM, from 6 nM to 7 nM, from 7 nM to 8 nM, from 8 nM to 9 nM, from 9 nM to 10 nM, from 10 nM to 20 nM, from 20 nM to 30 nM, from 30 nM to 40 nM, from 40 nM to 50 nM, from 50 nM to 60 nM, from 60 nM to 70 nM, from 70 nM to 80 nM, from 80 nM to 90 nM, from 90 nM to 100 nM, from 100 nM to 200 nM, from 200 nM to 300 nM, from 300 nM to 400 nM, from 400 nM to 500 nM, from 500 nM to 600 nM, from 600 nM to 700 nM, from 700 nM to 800 nM, from 800 nM to 900 nM, from 900 nM to 1 μM, from 1 μM to 2 μM, from 2 μM to 3 μM, from 3 μM to 4 μM, from 4 μM to 5 μM, from 5 μM to 6 μM, from 6 μM to 7 μM, from 7 μM to 8 μM, from 8 μM to 9 μM, from 9 μM to 10 μM, from 10 μM to 100 μM, from 100 μM to 1 mM, from 1 nM to 10 nM, from 1 nM to 100 nM, from 1 nM to 1 μM, from 1 nM to 10 μM, from 1 nM to 100 μM, from 1 nM to 1 mM, from 10 nM to 100 nM, from 10 nM to 1 μM, from 10 nM to 10 μM, from 10 nM to 100 μM, from 10 nM to 1 mM, from 100 nM to 1 μM, from 100 nM to 10 μM, from 100 nM to 100 μM, from 100 nM to 1 mM, from 1 μM to 10 μM, from 1 μM to 100 μM, from 1 μM to 1 mM, from 10 μM to 100 μM, from 10 μM to 1 mM, or from 100 μM to 1 mM. In some embodiments, the sample comprises a target nucleic acid at a concentration of from 20 nM to 200 μM, from 50 nM to 100 μM, from 200 nM to 50 μM, from 500 nM to 20 μM, or from 2 μM to 10 μM. In some embodiments, the target nucleic acid is not present in the sample.

In some embodiments, the sample comprises fewer than 10 copies, fewer than 100 copies, fewer than 1000 copies, fewer than 10,000 copies, fewer than 100,000 copies, or fewer than 1,000,000 copies of a target nucleic acid sequence. In some embodiments, the sample comprises from 10 copies to 100 copies, from 100 copies to 1000 copies, from 1000 copies to 10,000 copies, from 10,000 copies to 100,000 copies, from 100,000 copies to 1,000,000 copies, from 10 copies to 1000 copies, from 10 copies to 10,000 copies, from 10 copies to 100,000 copies, from 10 copies to 1,000,000 copies, from 100 copies to 10,000 copies, from 100 copies to 100,000 copies, from 100 copies to 1,000,000 copies, from 1,000 copies to 100,000 copies, or from 1,000 copies to 1,000,000 copies of a target nucleic acid sequence. In some embodiments, the sample comprises from 10 copies to 500,000 copies, from 200 copies to 200,000 copies, from 500 copies to 100,000 copies, from 1000 copies to 50,000 copies, from 2000 copies to 20,000 copies, from 3000 copies to 10,000 copies, or from 4000 copies to 8000 copies. In some embodiments, the target nucleic acid is not present in the sample.

A number of target nucleic acid populations are consistent with the methods and compositions disclosed herein. Some methods described herein can detect two or more target nucleic acid populations present in the sample in various concentrations or amounts. In some cases, the sample has at least 2 target nucleic acid populations. In some cases, the sample has at least 3, 4, 5, 6, 7, 8, 9, 10, 20, 30, 40, or 50 target nucleic acid populations. In some cases, the sample has from 3 to 50, from 5 to 40, or from 10 to 25 target nucleic acid populations. In some cases, the method detects target nucleic acid populations that are present at least at one copy per 10¹ non-target nucleic acids, 10² non-target nucleic acids, 10³ non-target nucleic acids, 10⁴ non-target nucleic acids, 10⁵ non-target nucleic acids, 10⁶ non-target nucleic acids, 10⁷ non-target nucleic acids, 10⁸ non-target nucleic acids, 10⁹ non-target nucleic acids, or 10¹⁰ non-target nucleic acids. The target nucleic acid populations can be present at different concentrations or amounts in the sample.

In some embodiments, the target nucleic acid as disclosed herein can activate the programmable nuclease to initiate sequence-independent cleavage of a nucleic acid-based reporter (e.g., a reporter comprising a DNA sequence, a reporter comprising an RNA sequence, or a reporter comprising DNA and RNA). For example, a programmable nuclease of the present disclosure is activated by a target DNA to cleave reporters having an RNA (also referred to herein as an “RNA reporter”). Alternatively, a programmable nuclease of the present disclosure is activated by a target RNA to cleave reporters having an RNA. Alternatively, a programmable nuclease of the present disclosure is activated by a target DNA to cleave reporters having a DNA (also referred to herein as a “DNA reporter”). The RNA reporter can comprise a single-stranded RNA labelled with a detection moiety or can be any RNA reporter as disclosed herein. The DNA reporter can comprise a single-stranded DNA labelled with a detection moiety or can be any DNA reporter as disclosed herein.

In some embodiments, the target nucleic acid as described in the methods herein does not initially comprise a PAM sequence. However, any target nucleic acid of interest may be generated using the methods described herein to comprise a PAM sequence, and thus be a PAM target nucleic acid. A PAM target nucleic acid, as used herein, refers to a target nucleic acid that has been amplified to insert a PAM sequence that is recognized by a CRISPR/Cas system.

In some embodiments, the target nucleic acid is in a cell. In some embodiments, the cell is a single-cell eukaryotic organism; a plant cell an algal cell; a fungal cell; an animal cell; a cell from an invertebrate animal; a cell from a vertebrate animal such as fish, amphibian, reptile, bird, and mammal; or a cell from a mammal such as a human, a non-human primate, an ungulate, a feline, a bovine, an ovine, and a caprine. In preferred embodiments, the cell is a eukaryotic cell. In preferred embodiments, the cell is a mammalian cell, a human cell, or a plant cell.

Any of the above disclosed samples are consistent with the methods, compositions, reagents, enzymes, and kits disclosed herein and can be used as a companion diagnostic with any of the diseases disclosed herein, or can be used in reagent kits, point-of-care diagnostics, or over-the-counter diagnostics.

### Methods of Modifying or Editing a Target Nucleic Acid Sequence

The disclosure provides compositions and methods for modifying or editing a target nucleic acid sequence. In some embodiments, the target nucleic acid sequence is associated with (e.g., causes, at least in part) a disease or disorder described herein, including a liver disease or disorder, an eye disease or disorder, cystic fibrosis, or a muscle disease or disorder. In some examples, the target nucleic acid comprises at least a portion of any one of the following genes: DNMT1, HPRT1, RPL32P3, CCR5, FANCF, GRIN2B, EMX1, AAVS1, ALKBH5, CLTA, CDK11, CTNNB1, AXIN1, LRP6, TBK1, BAP1, TLE3, PPM1A, BCL2L2, SUFU, RICTOR, VPS35, TOP1, SIRT1, PTEN, MMD, PAQR8, H2AX, POU5F1, OCT4, SYS1, ARFRP1, TSPAN14, EMC2, EMC3, SEL1L, DERL2, UBE2G2, UBE2J1, HRD1, PCSK9, BAK1 and CFTR. In some embodiments, the target nucleic acid comprises at least a portion of a PCSK9 gene. In some embodiments, the PCSK9 gene comprises a mutation associated with a liver disease or disorder. In some embodiments, the target nucleic acid comprises at least a portion of a BAK1 gene. In some embodiments, the BAK1 gene comprises a mutation associated with an eye disease or disorder. In some embodiments, the target nucleic acid comprises at least a portion of a CFTR gene. In some embodiments, the CFTR gene comprises a mutation associated with cystic fibrosis. In some embodiments, the CFTR gene comprises a delta F508 mutation. Compositions and methods of the disclosure can be used for introducing a site-specific cleavage in a target nucleic acid sequence. The site-specific cleavage can be a double-strand cleavage. The site-specific cleavage can be a single-strand cleavage (e.g. nicking). The modification can result in introducing a mutation (e.g., point mutations, deletions) in a target nucleic acid. The modification can result in removing a disease-causing mutation in a nucleic acid sequence. Methods of the disclosure can be targeted to any locus in a genome of a cell. They can generate point mutations, deletions, null mutations, or tissue-specific mutations in a target nucleic acid sequence. A complex comprising a programmable nuclease and guide nucleic acid of the disclosure can be used to generate gene knock-out, gene knock-in, gene editing, gene tagging, or a combination thereof. In some embodiments, the activity of a nuclease, such as a cleavage product, may be analyzed using gel electrophoresis or nucleic acid sequencing.

The methods described herein (e.g., methods of introducing a nick or a double-stranded break into a target nucleic acid) may be used to edit or modify a target nucleic acid. Methods of modifying a target nucleic acid may use the compositions comprising a programmable nuclease and a gRNA as described herein. Modifying a target nucleic acid may comprise one or more of cleaving the target nucleic acid, deleting one or more nucleotides of the target nucleic acid, inserting one or more nucleotides into the target nucleic acid, mutating one or more nucleotides of the target nucleic acid, or modifying (e.g., methylating, demethylating, deaminating, or oxidizing) of one or more nucleotides of the target nucleic acid.

In some embodiments, modifying a target nucleic acid comprises genome editing. Genome editing may comprise modifying a genome, chromosome, plasmid, or other genetic material of a cell or organism. In some embodiments the genome, chromosome, plasmid, or other genetic material of the cell or organism is modified in vivo. In some embodiments the genome, chromosome, plasmid, or other genetic material of the cell or organism is modified in a cell. In some embodiments the genome, chromosome, plasmid, or other genetic material of the cell or organism is modified in vitro. For example, a plasmid may be modified in vitro using a composition described herein and introduced into a cell or organism. In some embodiments, modifying a target nucleic acid may comprise deleting a sequence from a target nucleic acid. For example, a mutated sequence or a sequence associated with a disease may be removed from a target nucleic acid. In some embodiments, modifying a target nucleic acid may comprise replacing a sequence in a target nucleic acid with a second sequence. For example, a mutated sequence or a sequence associated with a disease may be replaced with a second sequence lacking the mutation or that is not associated with the disease. In some embodiments, modifying a target nucleic acid may comprise introducing a sequence into a target nucleic acid. For example, a beneficial sequence or a sequence that may reduce or eliminate a disease may inserted into the target nucleic acid.

In some embodiments, the present disclosure provides methods and compositions for editing a target nucleic acid sequence comprising a programmable nuclease capable of introducing a double-strand break in a double stranded DNA (dsDNA) target sequence. The programmable nuclease can be coupled to a guide nucleic acid that targets a particular region of interest in the dsDNA. A double-strand break can be repaired and rejoined by non-homologous end joining (NHEJ) or homology directed repair (HDR). Thus, a programmable nuclease capable of introducing a double-strand break as disclosed herein can be useful in a genome editing method, for example, used for therapeutic applications to treat a disease or disorder, or for agricultural applications. Such diseases or disorders that can be treated by the methods and compositions described herein include a liver disease or disorder, an eye disease or disorder, cystic fibrosis, or a muscle disease or disorder. CasΦ programmable nuclease disclosed herein can be used for genome editing purposes to generate double strand breaks in order to excise a region of DNA and subsequently introduce a region of DNA (e.g., donor DNA) into the excised region.

In some embodiments, the present disclosure provides methods and compositions for modifying or editing a target nucleic acid sequence comprising two or more programmable nickases. For example, modifying a target nucleic acid may comprise introducing a two or more single-stranded breaks in the target nucleic acid. In some embodiments, a break may be introduced by contacting a target nucleic acid with a programmable nickase and a guide nucleic acid. The guide nucleic acid may bind to the programmable nickase and hybridize to a region of the target nucleic acid, thereby recruiting the programmable nickase to the region of the target nucleic acid. Binding of the programmable nickase to the guide nucleic acid and the region of the target nucleic acid may activate the programmable nickase, and the programmable nickase may introduce a break (e.g., a single stranded break) in the region of the target nucleic acid. In some embodiments, modifying a target nucleic acid may comprise introducing a first break in a first region of the target nucleic acid and a second break in a second region of the target nucleic acid. For example, modifying a target nucleic acid may comprise contacting a target nucleic acid with a first guide nucleic acid that binds to a first programmable nickase and hybridizes to a first region of the target nucleic acid and a second guide nucleic acid that binds to a second programmable nickase and hybridizes to a second region of the target nucleic acid. The first programmable nickase may introduce a first break in a first strand at the first region of the target nucleic acid, and the second programmable nickase may introduce a second break in a second strand at the second region of the target nucleic acid. In some embodiments, a segment of the target nucleic acid between the first break and the second break may be removed, thereby modifying the target nucleic acid. In some embodiments, a segment of the target nucleic acid between the first break and the second break may be replaced (e.g., with an insert sequence), thereby modifying the target nucleic acid.

The methods of the disclosure can use HDR or NHEJ. Following cleavage of a targeted genomic sequence, one of two alternative DNA repair mechanisms can restore chromosomal integrity: non-homologous end joining (NHEJ) which can generate insertions and/or deletions of a few base-pairs of DNA at the cut site. Alternatively, the cell can employ homology-directed repair (HDR), which can correct the lesion via an additional DNA template (e.g., donor) that spans the cut site. In some instances, the methods of the disclosure use microhomology-mediated end-joining (MMEJ).

Methods and compositions of the disclosure can be used to insert a donor polynucleotide into a target nucleic acid sequence. A donor polynucleotide can comprise a segment of nucleic acid to be integrated at a target genomic locus. The donor polynucleotide can comprise one or more polynucleotides of interest. The donor polynucleotide can comprise one or more expression cassettes. The expression cassette can comprise a donor polynucleotide of interest, a polynucleotide encoding a selection marker and/or a reporter gene, and regulatory components that influence expression.

The donor polynucleotide can comprise a genomic nucleic acid. The genomic nucleic acid can be derived from an animal, a mouse, a human, a non-human, a rodent, a non-human, a rat, a hamster, a rabbit, a pig, a bovine, a deer, a sheep, a goat, a chicken, a cat, a dog, a ferret, a primate (e.g., marmoset, rhesus monkey), domesticated mammal or an agricultural mammal, an avian, a bacterium, a archaeon, a virus, or any other organism of interest or a combination thereof. The donor polynucleotide may be synthetic.

Donor polynucleotides of any suitable size can be integrated into a genome. In some embodiments, the donor polynucleotide integrated into a genome is less than 3, about 3, 3.5, 4, 4.5, 5, 5.5, 6, 6.5, 7, 7.5, 8, 8.5, 9, 9.5, 10, 10.5, 11, 11.5, 12, 12.5, 13, 13.5, 14, 14.5, 15, 16, 17, 18, 19, 20, 25, 30, 35, 40, 45, 50, 100, 150, 200, 250, 300, 350, 400, 450, 500 or more than 500 kilobases (kb) in length. In some embodiments, the donor polynucleotide integrated into a genome is at least about 2, 2.5, 3, 3.5, 4, 4.5, 5, 5.5, 6, 6.5, 7, 7.5, 8, 8.5, 9, 9.5, 10, 10.5, 11, 11.5, 12, 12.5, 13, 13.5, 14, 14.5, 15, 16, 17, 18, 19, 20, 25, 30, 35, 40, 45, 50, 100, 150, 200, 250, 300, 350, 400, 450, 500 or more than 500 kb in length. In some embodiments, the donor polynucleotide integrated into a genome is up to about 3, 3.5, 4, 4.5, 5, 5.5, 6, 6.5, 7, 7.5, 8, 8.5, 9, 9.5, 10, 10.5, 11, 11.5, 12, 12.5, 13, 13.5, 14, 14.5, 15, 16, 17, 18, 19, 20, 25, 30, 35, 40, 45, 50, 100, 150, 200, 250, 300, 350, 400, 450, 500 or more than 500 kb in length.

The donor polynucleotide can be flanked by site-specific recombination target sequences (e.g., 5′ and 3′ homology arms) on a targeting vector. The length of a homology arm may be from about 50 to about 1000 bp. The length of a homology arm may be from about 400 to about 1000 bp. A homology arm can be of any length that is sufficient to promote a homologous recombination event with a corresponding target site, including for example, from about 400 bp to about 500 bp, from about 500 bp to about 600 bp, from about 600 bp to about 700 bp, from about 700 bp to about 800 bp, from about 800 bp to about 900 bp, or from about 900 bp to about 1000 bp. In preferred embodiments, the length of a homology arm may be from about 200 to about 300 bp. The sum total of 5′ and 3′ homology arms can be about 0.5 kb, 1 kb, 1.5 kb, 2 kb, 3 kb, 4 kb, 5 kb, 6 kb, 7 kb, 8 kb, 9 kb, about 0.5 kb to about 1 kb, about 1 kb to about 1.5 kb, about 1.5 kb to about 2 kb, about 2 kb to about 3 kb, about 3 kb to about 4 kb, about 4 kb to about 5 kb, about 5 kb to about 6 kb, about 6 kb to about 7 kb, about 8 kb to about 9 kb, or is at least 10 kb.

In some embodiments, the donor polynucleotide comprises one or more phosphorothioate bonds between nucleobases. In some embodiments, one or more of the first five 5′ nucleobases of the donor polynucleotide are linked by phosphorothioate bonds. In some embodiments, one or more of the five nucleobases at the 3′ end of the donor polynucleotide are linked by phosphorothioate bonds. In some embodiments, one or more of the first three 5′ nucleobases of the donor polynucleotide are linked by phosphorothioate bonds. In some embodiments, one or more of the three nucleobases at the 3′ end of the donor polynucleotide are linked by phosphorothioate bonds. In preferred embodiments, the two nucleobases at 5′ end of the donor polynucleotide are linked by a phosphorothioate bond. In some embodiments, the two nucleobases at the 3′ end of the donor polynucleotide are linked by a phosphorothioate bond. In more preferred embodiments, the two nucleobases at 5′ end of the donor polynucleotide are linked by a phosphorothioate bond and the two nucleobases at the 3′ end of the donor polynucleotide are linked by a phosphorothioate bond.

Examples of site-specific recombinases that can be used include, but are not limited to, Cre, Flp, and Dre recombinases. The site-specific recombinase can be introduced into the cell by any means, including by introducing the recombinase polypeptide into the cell or by introducing a polynucleotide encoding the site-specific recombinase into the host cell. The polynucleotide encoding the site-specific recombinase can be located within the insert polynucleotide or within a separate polynucleotide. The site-specific recombinase can be operably linked to a promoter active in the cell including, for example, an inducible promoter, a promoter that is endogenous to the cell, a promoter that is heterologous to the cell, a cell-specific promoter, a tissue-specific promoter, or a developmental stage-specific promoter. Site-specific recombination target sequences which can flank the insert polynucleotide or any polynucleotide of interest in the insert polynucleotide can include, but are not limited to, loxP, lox511, loχ2272, loχ66, lox71, loxM2, lox5171, FRT, FRT11, FRT71, attp, att, FRT, rox, and a combination thereof.

The target nucleic acid may comprise one or more of a genome, a chromosome, a plasmid, a gene, a promoter, an untranslated region, an open reading frame, an intron, an exon, or an operator. The target nucleic acid may comprise a segment of one or more of a genome, a chromosome, a plasmid, a gene, a promoter, an untranslated region, an open reading frame, an intron, an exon, or an operator. In some embodiments, the target nucleic acid may be part of a cell or an organism. In some embodiments, the target nucleic acid may be a cell-free genetic component.

In some embodiments, gene modifying or gene editing is achieved by fusing a programmable nuclease such as a CasΦ protein to a heterologous sequence. The heterologous sequence can be a suitable fusion partner, e.g., a polypeptide that provides recombinase activity by acting on the target nucleic acid sequence. In some embodiments, the fusion protein comprises a programmable nuclease such as a CasΦ protein fused to a heterologous sequence by a linker.

The heterologous sequence or fusion partner can be a site specific recombinase. The site specific recombinase can have recombinase activity. Examples of site-specific recombinases that can be used include, but are not limited to, Cre, Hin, Tre, and FLP recombinases. The heterologous sequence or fusion partner can be a recombinase catalytic domain. The recombinase catalytic domains can be from, for example, a tyrosine recombinase, a serine recombinase, a Gin recombinase, a Hin recombinase, a R recombinase, a Sin recombinase, a Tn3 recombinase, a γδ recombinase, a Cre recombinase, a FLP recombinase, or a phC31 integrase.

The heterologous sequence or fusion partner can be fused to the C-terminus, N-terminus, or an internal portion (e.g., a portion other than the N- or C-terminus) of the programmable nuclease, for example a dead CasΦ polypeptide.

The heterologous sequence or fusion partner can be fused to the programmable nuclease by a linker. A linker can be a peptide linker or a non-peptide linker. In some embodiments, the linker is an XTEN linker. In some embodiments, the linker comprises one or more repeats a tri-peptide GGS. In some embodiments, the linker is from 1 to 100 amino acids in length. In some embodiments, the linker is more 100 amino acids in length. In some embodiments, the linker is from 10 to 27 amino acids in length. A non-peptide linker can be a polyethylene glycol (PEG), polypropylene glycol (PPG), co-poly(ethylene/propylene) glycol, polyoxyethylene (POE), polyurethane, polyphosphazene, polysaccharides, dextran, polyvinyl alcohol, polyvinylpyrrolidones, polyvinyl ethyl ether, polyacryl amide, polyacrylate, polycyanoacrylates, lipid polymers, chitins, hyaluronic acid, heparin, or an alkyl linker.

In some embodiments, the CasΦ protein can comprise an enzymatically inactive and/or “dead” (abbreviated by “d”) programmable nuclease in combination (e.g., fusion) with a polypeptide comprising recombinase activity. Although a programmable CasΦ nuclease normally has nuclease activity, in some embodiments, a programmable CasΦ nuclease does not have nuclease activity.

A programmable nuclease can comprise a modified form of a wild type counterpart. The modified form of the wild type counterpart can comprise an amino acid change (e.g., deletion, insertion, or substitution) that reduces the nucleic acid-cleaving activity of the programmable nuclease. For example, a nuclease domain (e.g., RuvC domain) of a CasΦ polypeptide can be deleted or mutated so that it is no longer functional or comprises reduced nuclease activity. The modified form of the programmable nuclease can have less than 90%, less than 80%, less than 70%, less than 60%, less than 50%, less than 40%, less than 30%, less than 20%, less than 10%, less than 5%, or less than 1% of the nucleic acid-cleaving activity of the wild-type counterpart.

The modified form of a programmable nuclease can have no substantial nucleic acid-cleaving activity. When a programmable nuclease is a modified form that has no substantial nucleic acid-cleaving activity, it can be referred to as enzymatically inactive and/or dead. A dead CasΦ polypeptide (e.g., dCasΦ) can bind to a target nucleic acid sequence but may not cleave the target nucleic acid sequence. A dCasΦ polypeptide can associate with a guide nucleic acid to activate or repress transcription of a target nucleic acid sequence.

In some embodiments, a programmable nuclease is a dead CasΦ polypeptide. A dead CasΦ polypeptide can comprise at least 70%, at least 75%, at least 80%, at least 85%, at least 90%, at least 92%, at least 95%, at least 97%, at least 99%, or 100% sequence identity with any one of SEQ ID NO: 1-SEQ ID NO: 47, SEQ ID NO. 105, and SEQ ID NO 107. In some embodiments, a programmable nuclease is a dead CasΦ polypeptide comprising at least 85% sequence identity to any one of SEQ ID NO: 1-SEQ ID NO: 47, SEQ ID NO. 105, and SEQ ID NO 107. In some embodiments, a programmable nuclease is a dead CasΦ polypeptide comprising at least 90% sequence identity to any one of SEQ ID NO: 1-SEQ ID NO: 47, SEQ ID NO. 105, and SEQ ID NO 107. In some embodiments, a programmable nuclease is a dead CasΦ polypeptide comprising at least 95% sequence identity to any one of SEQ ID NO: 1-SEQ ID NO: 47, SEQ ID NO. 105, and SEQ ID NO 107. In some embodiments, a programmable nuclease is a dead CasΦ polypeptide comprising at least 98% sequence identity to any one of SEQ ID NO: 1-SEQ ID NO: 47, SEQ ID NO. 105, and SEQ ID NO 107.

A deadCasΦ (also referred to herein as “dCasΦ”) polypeptide can form a ribonucleoprotein complex with a guide nucleic acid. The guide nucleic acid can comprise a crRNA sequence comprising at least 70%, at least 80%, at least 90%, at least 92%, at least 95%, at least 97%, or at least 99%, or 100% sequence identity to any one of SEQ ID NO: 48-SEQ ID NO: 86, or a reverse complement thereof.

Enzymatically inactive can refer to a polypeptide that can bind to a nucleic acid sequence in a polynucleotide in a sequence-specific manner, but may not cleave a target polynucleotide. An enzymatically inactive site-directed polypeptide can comprise an enzymatically inactive domain (e.g. a programmable nuclease domain). Enzymatically inactive can refer to no activity. Enzymatically inactive can refer to substantially no activity. Enzymatically inactive can refer to essentially no activity. Enzymatically inactive can refer to an activity less than 1%, less than 2%, less than 3%, less than 4%, less than 5%, less than 6%, less than 7%, less than 8%, less than 9%, or less than 10% activity compared to a wild-type exemplary activity (e.g., nucleic acid cleaving activity, wild-type CasΦ activity).

In further embodiments, methods of modifying cells are provided. In some embodiments, a method of modifying a cell comprising a target nucleic acid wherein the method comprises introducing a programmable CasΦ nuclease or variant thereof disclosed herein to the cell, wherein the programmable CasΦ nuclease or variant cleaves or modifies the target nucleic acid.

Modified cells obtained or obtainable by the methods described herein are provided. In some embodiments, a modified cell is obtained or is obtained by a method of modifying a cell disclosed herein.

In some embodiments, a CasΦ polypeptide disclosed herein is expressed in a cell. In some embodiments, a CasΦ polypeptide disclosed herein complexed with a guide nucleic is expressed in a cell. In some embodiments, a method disclosed herein comprises modifying or editing a cell. In some embodiments, a modified cell is provided wherein a cell is modified by a CasΦ polypeptide disclosed herein. In some embodiments, a cell is provided wherein the cell comprises a CasΦ polypeptide disclosed herein.

### Methods of Nicking of a Target Nucleic Acid

Disclosed herein are methods of introducing a break into a target nucleic acid. In some embodiments, the break may be a single stranded break (e.g., a nick). The programmable nickases disclosed herein and a gRNA disclosed herein may be used to introduce a single-stranded break into a target nucleic acid, for example a single stranded break in a double-stranded DNA.

A method of introducing a break into a target nucleic acid may comprise contacting the target nucleic acid with a first guide nucleic acid (e.g., a guide nucleic acid comprising a region that binds to a first programmable nickase) and a second guide nucleic acid (e.g., a guide nucleic acid comprising a region that binds to a second programmable nickase). The first guide nucleic acid may comprise an additional region that binds to the target nucleic acid, and the second guide nucleic acid may comprise an additional region that binds to the target nucleic acid. The additional region of the first guide nucleic acid and the additional region of the second guide nucleic acid may bind opposing strands of the target nucleic acid.

In some embodiments, a programmable nickase of the disclosure can cleave a non-target strand of a double-stranded target nucleic acid (e.g., DNA). In some embodiments, the programmable nickase may not cleave the target strand of the double-stranded target nucleic acid (e.g., DNA). The strand of a double-stranded target nucleic acid that is complementary to and hybridizes with the guide nucleic acid can be called the target strand. The strand of the double-stranded target DNA that is complementary to the target strand, and therefore is not complementary to the guide nucleic acid can be called non-target strand.

The temperature at which a ribonucleoprotein (RNP) complex comprising a programmable nuclease and a guide nucleic acid is formed (i.e. the RNP complexing temperature) can affect the nickase activity of the programmable nuclease. For example, an RNP complex formed at room temperature can have a greater nickase activity than an RNP complex formed at 37° C. In some cases, the RNP complex can be formed at room temperature, for example, from about 20° C. to 22° C. In some cases, the RNP complex can be formed at, for example, about 15° C., about 16° C., about 17° C., about 18° C., about 19° C., about 20° C., about 21° C., about 22° C., about 23° C., about 24° C., or about 25° C.

In some embodiments, a programmable nuclease may exhibit at least about 1.1-fold, at least about 1.2-fold, at least about 1.3-fold, at least about 1.4-fold, at least about 1.5-fold, at least about 1.6-fold, at least about 1.7-fold, at least about 1.8-fold, at least about 1.9-fold, at least about 2-fold, at least about 2.1-fold, at least about 2.2-fold, at least about 2.3-fold, at least about 2.4-fold, at least about 2.5-fold, at least about 2.6-fold, at least about 2.7-fold, at least about 2.8-fold, at least about 2.9-fold, at least about 3-fold, at least about 3.5-fold, at least about 4-fold, at least about 4.5-fold, at least about 5-fold, at least about 5.5-fold, at least about 6-fold, at least about 7-fold, at least about 8-fold, at least about 9-fold, at least about 10-fold, at least about 15-fold, at least about 20-fold, at least about 30-fold, at least about 40-fold, or at least about 50-fold greater nicking activity when complexed with a guide RNA at room temperature as compared to when complexed at 37° C.

The crRNA repeat sequence of a guide nucleic acid can affect the nickase activity of a programmable nuclease. For example, a programmable nuclease can comprise enhanced or greater nickase activity when complexed with guide nucleic acids comprising certain crRNA repeat sequences. For example, a programmable nuclease can comprise greater nickase activity when complexed with a guide RNA comprising a crRNA repeat sequence of CasΦ.18 as shown in TABLE 2. In another example, a programmable nuclease can comprise greater nickase activity when complexed with a guide RNA comprising a crRNA repeat sequence of CasΦ.7 as shown in TABLE 2. In some embodiments, a programmable nuclease may exhibit at least about 1.1-fold, at least about 1.2-fold, at least about 1.3-fold, at least about 1.4-fold, at least about 1.5-fold, at least about 1.6-fold, at least about 1.7-fold, at least about 1.8-fold, at least about 1.9-fold, at least about 2-fold, at least about 2.1-fold, at least about 2.2-fold, at least about 2.3-fold, at least about 2.4-fold, at least about 2.5-fold, at least about 2.6-fold, at least about 2.7-fold, at least about 2.8-fold, at least about 2.9-fold, at least about 3-fold, at least about 3.5-fold, at least about 4-fold, at least about 4.5-fold, at least about 5-fold, at least about 5.5-fold, at least about 6-fold, at least about 7-fold, at least about 8-fold, at least about 9-fold, at least about 10-fold, at least about 15-fold, at least about 20-fold, at least about 30-fold, at least about 40-fold, or at least about 50-fold greater nicking activity when complexed with a guide RNA comprising a specific crRNA repeat sequence as compared to when in a complex with a guide RNA comprising another crRNA repeat sequence.

The programmable nucleases disclosed herein may exhibit cis-cleavage activity or target cleavage activity. Target cleavage activity may refer to the cleavage of a target nucleic acid by the programmable nuclease. In some cases, the cis-cleavage activity results in double-stranded breaks in the target nucleic acids. In some cases, the cis-cleavage activity results in single-stranded breaks in the target nucleic acids. In some cases, the cis-cleavage activity produces a mixture of double- and single-stranded breaks in the target nucleic acids. In further cases, the rates of cis-cleavage double- and single-strand break formation may be dependent on the sequence of the guide nucleic acid. In some cases, the ratio of cis-cleavage double- and single-strand break formation may be dependent on the sequence of the guide nucleic acid. In some cases, the ratio or rate of cis-cleavage double- and single-strand break formation may be dependent on the repeat sequence of the crRNA of the guide nucleic acid. In some cases, the ratio or rate of cis-cleavage double- and single-strand break formation may be dependent on the temperature at which the ribonucleoprotein complex comprising the programmable nuclease and the guide nucleic acid are complexed.

A programmable nuclease for use in modifying a target nucleic acid may have greater nicking activity as compared to double stranded cleavage activity. In some embodiments, a programmable nuclease may exhibit at least about 1.1-fold, at least about 1.2-fold, at least about 1.3-fold, at least about 1.4-fold, at least about 1.5-fold, at least about 1.6-fold, at least about 1.7-fold, at least about 1.8-fold, at least about 1.9-fold, at least about 2-fold, at least about 2.1-fold, at least about 2.2-fold, at least about 2.3-fold, at least about 2.4-fold, at least about 2.5-fold, at least about 2.6-fold, at least about 2.7-fold, at least about 2.8-fold, at least about 2.9-fold, at least about 3-fold, at least about 3.5-fold, at least about 4-fold, at least about 4.5-fold, at least about 5-fold, at least about 5.5-fold, at least about 6-fold, at least about 7-fold, at least about 8-fold, at least about 9-fold, at least about 10-fold, at least about 15-fold, at least about 20-fold, at least about 30-fold, at least about 40-fold, or at least about 50-fold greater nicking activity as compared to double stranded cleavage activity.

In other cases, a programmable nuclease for use in modifying a target nucleic acid may have greater double stranded cleavage activity as compared to nicking activity. In some embodiments, a programmable nuclease may exhibit at least about 1.1-fold, at least about 1.2-fold, at least about 1.3-fold, at least about 1.4-fold, at least about 1.5-fold, at least about 1.6-fold, at least about 1.7-fold, at least about 1.8-fold, at least about 1.9-fold, at least about 2-fold, at least about 2.1-fold, at least about 2.2-fold, at least about 2.3-fold, at least about 2.4-fold, at least about 2.5-fold, at least about 2.6-fold, at least about 2.7-fold, at least about 2.8-fold, at least about 2.9-fold, at least about 3-fold, at least about 3.5-fold, at least about 4-fold, at least about 4.5-fold, at least about 5-fold, at least about 5.5-fold, at least about 6-fold, at least about 7-fold, at least about 8-fold, at least about 9-fold, at least about 10-fold, at least about 15-fold, at least about 20-fold, at least about 30-fold, at least about 40-fold, or at least about 50-fold greater double stranded cleavage activity as compared to nicking activity.

In some embodiments, the nicking activity and double stranded cleavage activity of a programmable nuclease depend on the conditions and species present in the sample containing the programmable nuclease. In some cases, the nicking activity and double stranded cleavage activity of the programmable nuclease are responsive to the sequence of the crRNA present in the guide nucleic acid. In some cases, the ratio of nicking activity and double stranded cleavage activity can be modulated by changing the sequence of the crRNA present. In some cases, the nicking activity and double stranded cleavage activity of the programmable nuclease respond differently to changes in temperature (e.g., RNP complexing temperature), pH, osmolarity, buffer, target nucleic acid concentration, ionic strength, and inhibitor concentration. In some embodiments, the ratio of nicking activity to cleavage activity by a programmable nuclease can be actively controlled by adjusting sample conditions and crRNA sequences.

### Methods of Regulating Gene Expression

In some embodiments, the disclosure provided methods and compositions for regulating gene expression. The methods and compositions can comprise use of an enzymatically inactive and/or “dead” (abbreviated by “d”) programmable nuclease in combination (e.g., fusion) with a polypeptide comprising transcriptional regulation activity. Although a programmable CasΦ nuclease normally has nuclease activity, in some embodiments, a programmable CasΦ nuclease does not have nuclease activity.

A programmable nuclease can comprise a modified form of a wild type counterpart. The modified form of the wild type counterpart can comprise an amino acid change (e.g., deletion, insertion, or substitution) that reduces the nucleic acid-cleaving activity of the programmable nuclease. For example, a nuclease domain (e.g., RuvC domain) of a CasΦ polypeptide can be deleted or mutated so that it is no longer functional or comprises reduced nuclease activity. The modified form of the programmable nuclease can have less than 90%, less than 80%, less than 70%, less than 60%, less than 50%, less than 40%, less than 30%, less than 20%, less than 10%, less than 5%, or less than 1% of the nucleic acid-cleaving activity of the wild-type counterpart. The modified form of a programmable nuclease can have no substantial nucleic acid-cleaving activity. When a programmable nuclease is a modified form that has no substantial nucleic acid-cleaving activity, it can be referred to as enzymatically inactive and/or dead. A dead CasΦ polypeptide (e.g., dCasΦ) can bind to a target nucleic acid sequence but may not cleave the target nucleic acid sequence. A dCasΦ polypeptide can associate with a guide nucleic acid to activate or repress transcription of a target nucleic acid sequence.

In some embodiments, the disclosure provides a method of selectively modulating transcription of a gene in a cell. The method can comprise introducing into a cell a (i) fusion polypeptide comprising a dCasΦ polypeptide and a polypeptide comprising transcriptional regulation activity, or a nucleic acid comprising a nucleotide sequence encoding the fusion polypeptide, wherein the dCasΦ polypeptide is enzymatically inactive or exhibits reduced nucleic acid cleavage activity; and ii) a guide nucleic acid, or a nucleic acid comprising a nucleotide sequence encoding the guide nucleic acid.

In some embodiments, a programmable nuclease is a dead CasΦ polypeptide. A dead CasΦ polypeptide can comprise at least 70%, at least 75%, at least 80%, at least 85%, at least 90%, at least 92%, at least 95%, at least 97%, at least 99%, or 100% sequence identity with any one of SEQ ID NO: 1-SEQ ID NO: 47, SEQ ID NO. 105, and SEQ ID NO 107. In some embodiments, a programmable nuclease is a dead CasΦ polypeptide comprising at least 85% sequence identity to any one of SEQ ID NO: 1-SEQ ID NO: 47, SEQ ID NO. 105, and SEQ ID NO 107. In some embodiments, a programmable nuclease is a dead CasΦ polypeptide comprising at least 90% sequence identity to any one of SEQ ID NO: 1-SEQ ID NO: 47, SEQ ID NO. 105, and SEQ ID NO 107. In some embodiments, a programmable nuclease is a dead CasΦ polypeptide comprising at least 95% sequence identity to any one of SEQ ID NO: 1-SEQ ID NO: 47, SEQ ID NO. 105, and SEQ ID NO 107. In some embodiments, a programmable nuclease is a dead CasΦ polypeptide comprising at least 98% sequence identity to any one of SEQ ID NO: 1-SEQ ID NO: 47, SEQ ID NO. 105, and SEQ ID NO 107.

A deadCasΦ (also referred to herein as “dCasΦ”) polypeptide can form a ribonucleoprotein complex with a guide nucleic acid. The guide nucleic acid can comprise a crRNA sequence comprising at least 70%, at least 80%, at least 90%, at least 92%, at least 95%, at least 97%, or at least 99%, or 100% sequence identity to any one of SEQ ID NO: 48-SEQ ID NO: 86, or a reverse complement thereof.

Enzymatically inactive can refer to a polypeptide that can bind to a nucleic acid sequence in a polynucleotide in a sequence-specific manner, but may not cleave a target polynucleotide. An enzymatically inactive site-directed polypeptide can comprise an enzymatically inactive domain (e.g. a programmable nuclease domain). Enzymatically inactive can refer to no activity. Enzymatically inactive can refer to substantially no activity. Enzymatically inactive can refer to essentially no activity. Enzymatically inactive can refer to an activity less than 1%, less than 2%, less than 3%, less than 4%, less than 5%, less than 6%, less than 7%, less than 8%, less than 9%, or less than 10% activity compared to a wild-type exemplary activity (e.g., nucleic acid cleaving activity, wild-type CasΦ activity).

Transcription regulation can be achieved by fusing a programmable nuclease such as a dead CasΦ protein to a heterologous sequence. The heterologous sequence can be a suitable fusion partner, e.g., a polypeptide that provides an activity that increases, decreases, or otherwise regulates transcription by acting on the target nucleic acid sequence or on a polypeptide (e.g., a histone or other DNA-binding protein) associated with the target nucleic acid sequence. Non-limiting examples of suitable fusion partners include a polypeptide that provides for transcription activation activity, transcription repression activity, nuclease activity, transcription release factor activity, histone modification activity, histone acetyltransferase activity, nucleic acid association activity, DNA methylase activity, direct or indirect DNA demethylase activity, methyltransferase activity, demethylase activity, acetyltransferase activity, deacetylase activity, kinase activity, phosphatase activity, ubiquitin ligase activity, deubiquitinating activity, adenylation activity, deaminase activity, deadenylation activity, SUMOylating activity, deSUMOylating activity, ribosylation activity, deribosylation activity, myristoylation activity, or demyristoylation activity.

Illustrative modifications performed by a fusion polypeptide can comprise methylation, demethylation, acetylation, deacetylation, ubiquitination, deubiquitination, deamination, alkylation, depurination, oxidation, pyrimidine dimer formation, transposition, recombination, chain elongation, ligation, glycosylation. Phosphorylation, dephosphorylation, adenylation, deadenylation, SUMOylation, deSUMOylation, ribosylation, deribosylation, myristoylation, remodeling, cleavage, oxidoreduction, hydrolation, or isomerization.

The heterologous sequence or fusion partner can be fused to the C-terminus, N-terminus, or an internal portion (e.g., a portion other than the N- or C-terminus) of the programmable nuclease, for example a dead CasΦ polypeptide. Non-limiting examples of fusion partners include transcription activators, transcription repressors, histone lysine methyltransferases (KMT), Histone Lysine Demethylates, Histone lysine acetyltransferases (KAT), Histone lysine deacetylase, DNA methylases (adenosine or cytosine modification), deaminases, CTCF, periphery recruitment elements (e.g., Lamin A, Lamin B), and protein docking elements (e.g., FKBP/FRB).

Non-limiting examples of transcription activators include GAL4, VP16, VP64, and p65 subdomain (NFkappaB).

Non-limiting examples of transcription repressors include Kruippel associated box (KRAB or SKD), the Mad mSIN3 interaction domain (SID), and the ERF repressor domain (ERD).

Non-limiting examples of histone lysine methyltransferases (KMT) include members from KMT1 family (e.g., SUV39H1, SUV39H2, G9A, ESET/SETDB1, Clr4, Su(var)3-9), KMT2 family members (e.g., hSET1A, hSET1B, MLL 1 to 5, ASH1, and homologs (Trx, Trr, Ash1)), KMT3 family (SYMD2, NSD1), KMT4 (DOT1L and homologs), KMT5 family (Pr-SET7/8, SUV4-20H1, and homologs), KMT6 (EZH2), and KMT8 (e.g., RIZ1).

Non-limiting examples of Histone Lysine Demethylates (KDM) include members from KDM1 family (LSD1/BHC110, Splsd1/Swm1/Saf11 0, Su(var)3-3), KDM3 family (JHDM2a/b), KDM4 family (JMJD2A/JHDM3A, JMJD2B, JMJD2C/GASC1, JMJD2D, and homologs (Rph1)), KDM5 family (JARID1A/RBP2, JARID1B/PLU-1, JARIDIC/SMCX, JARID1D/SMCY, and homologs (Lid, Jhn2, Jmj2)), and KDM6 family (e.g., UTX, JMJD3).

Non-limiting examples of KAT include members of KAT2 family (hGCN5, PCAF, and homologs (dGCN5/PCAF, Gcn5), KAT3 family (CBP, p300, and homologs (dCBP/NEJ)), KAT4, KAT5, KAT6, KAT7, KAT8, and KAT13.

In some embodiments, the disclosure provides methods for increasing transcription of a target nucleic acid sequence. The transcription of a target nucleic acid sequence can increase by at least about 1.1 fold, at least about 1.2 fold, at least about 1.3 fold, at least about 1.4 fold, at least about 1.5 fold, at least about 1.6 fold, at least about 1.7 fold, at least about 1.8 fold, at least about 1.9 fold, at least about 2 fold, at least about 2.5 fold, at least about 3 fold, at least about 3.5 fold, at least about 4 fold, at least about 4.5 fold, at least about 5 fold, at least about 6 fold, at least about 7 fold, at least about 8 fold, at least about 9 fold, at least about 10 fold, at least about 12 fold, at least about 15 fold, at least about 20-fold, at least about 50-fold, at least about 70-fold, or at least about 100-fold compared to the level of transcription of the target nucleic acid sequence in the absence of a fusion polypeptide comprising a enzymatically inactive or enzymatically reduced programmable nuclease (e.g., dead CasΦ protein).

In some embodiments, the disclosure provides methods for decreasing transcription of a target nucleic acid sequence. The transcription of a target nucleic acid sequence can decrease by at least about 1.1 fold, at least about 1.2 fold, at least about 1.3 fold, at least about 1.4 fold, at least about 1.5 fold, at least about 1.6 fold, at least about 1.7 fold, at least about 1.8 fold, at least about 1.9 fold, at least about 2 fold, at least about 2.5 fold, at least about 3 fold, at least about 3.5 fold, at least about 4 fold, at least about 4.5 fold, at least about 5 fold, at least about 6 fold, at least about 7 fold, at least about 8 fold, at least about 9 fold, at least about 10 fold, at least about 12 fold, at least about 15 fold, at least about 20-fold, at least about 50-fold, at least about 70-fold, or at least about 100-fold compared to the level of transcription of the target nucleic acid sequence in the absence of a fusion polypeptide comprising a enzymatically inactive or enzymatically reduced programmable nuclease (e.g., dead Cas 12j protein).

### Method of Treating a Disorder

The compositions and methods described herein may be used to treat, prevent, or inhibit an ailment in a subject. The ailments may include diseases, cancers, genetic disorders, neoplasias, and infections. In some cases, the disease or disorder for treatment is a liver disease or disorder, an eye disease or disorder, cystic fibrosis, or a muscle disease or disorder. In some cases, the ailments are associated with one or more genetic sequences, including but not limited to 11-hydroxylase deficiency; 17,20-desmolase deficiency; 17-hydroxylase deficiency; 3-hydroxyisobutyrate aciduria; 3-hydroxysteroid dehydrogenase deficiency; 46,XY gonadal dysgenesis; AAA syndrome; ABCA3 deficiency; ABCC8-associated hyperinsulinism; aceruloplasminemia; achondrogenesis type 2; acral peeling skin syndrome; acrodermatitis enteropathica; adrenocortical micronodular hyperplasia; adrenoleukodystrophies; adrenomyeloneuropathies; Aicardi-Goutieres syndrome; Alagille disease; Alpers syndrome; alpha-mannosidosis; Alstrom syndrome; Alzheimer disease; amelogenesis imperfecta; amish type microcephaly; amyotrophic lateral sclerosis (ALS); anauxetic dysplasia; androgen insensitivity syndrome; Antley-Bixler syndrome; APECED, Apert syndrome, aplasia of lacrimal and salivary glands, argininemia, arrhythmogenic right ventricular dysplasia, Arts syndrome, ARVD2, arylsulfatase deficiency type metachromatic leokodystrophy, ataxia telangiectasia, autoimmune lymphoproliferative syndrome; autoimmune polyglandular syndrome type 1; autosomal dominant anhidrotic ectodermal dysplasia; autosomal dominant polycystic kidney disease; autosomal recessive microtia; autosomal recessive renal glucosuria; autosomal visceral heterotaxy; Bardet-Biedl syndrome; Bartter syndrome; basal cell nevus syndrome; Batten disease; benign recurrent intrahepatic cholestasis; beta-mannosidosis; Bethlem myopathy; Blackfan-Diamond anemia; blepharophimosis; Byler disease; C syndrome; CADASIL; carbamyl phosphate synthetase deficiency; cardiofaciocutaneous syndrome; Carney triad; carnitine palmitoyltransferase deficiencies; cartilage-hair hypoplasia; cblC type of combined methylmalonic aciduria; CD18 deficiency; CD3Z-associated primary T-cell immunodeficiency; CD40L deficiency; CDAGS syndrome; CDG1A; CDG1B; CDG1M; CDG2C; CEDNIK syndrome; central core disease; centronuclear myopathy; cerebral capillary malformation; cerebrooculofacioskeletal syndrome type 4; cerebrooculogacioskeletal syndrome; cerebrotendinous xanthomatosis; CHARGE association; cherubism; CHILD syndrome; chronic granulomatous disease; chronic recurrent multifocal osteomyelitis; citrin deficiency; classic hemochromatosis; CNPPB syndrome; cobalamin C disease; Cockayne syndrome; coenzyme Q10 deficiency; Coffin-Lowry syndrome; Cohen syndrome; combined deficiency of coagulation factors V; common variable immune deficiency; complete androgen insentivity; cone rod dystrophies; conformational diseases; congenital bile adid synthesis defect type 1; congenital bile adid synthesis defect type 2; congenital defect in bile acid synthesis type; congenital erythropoietic porphyria; congenital generalized osteosclerosis; Cornelia de Lange syndrome; Cousin syndrome; Cowden disease; COX deficiency; Crigler-Najjar disease; Crigler-Najjar syndrome type 1; Crisponi syndrome; Currarino syndrome; Curth-Macklin type ichthyosis hystrix; cutis laxa; cystic fibrosis; cystinosis; d-2-hydroxyglutaric aciduria; DDP syndrome; Dejerine-Sottas disease; Denys-Drash syndrome; desmin cardiomyopathy; desmin myopathy; DGUOK-associated mitochondrial DNA depletion; disorders of glutamate metabolism; distal spinal muscular atrophy type 5; DNA repair diseases; dominant optic atrophy; Doyne honeycomb retinal dystrophy; Duchenne muscular dystrophy; dyskeratosis congenita; Ehlers-Danlos syndrome type 4; Ehlers-Danlos syndromes; Elejalde disease; Ellis-van Creveld disease; Emery-Dreifuss muscular dystrophies; encephalomyopathic mtDNA depletion syndrome; enzymatic diseases; EPCAM-associated congenital tufting enteropathy; epidermolysis bullosa with pyloric atresia; exercise-induced hypoglycemia; facioscapulohumeral muscular dystrophy; Faisalabad histiocytosis; familial atypical mycobacteriosis; familial capillary malformation-arteriovenous; familial esophageal achalasia; familial glomuvenous malformation; familial hemophagocytic lymphohistiocytosis; familial mediterranean fever; familial megacalyces; familial schwannomatosisl; familial spina bifida; familial splenic asplenia/hypoplasia; familial thrombotic thrombocytopenic purpura; Fanconi disease; Feingold syndrome; FENIB; fibrodysplasia ossificans progressiva; FKTN; Francois-Neetens fleck corneal dystrophy; Frasier syndrome; Friedreich ataxia; FTDP-17; fucosidosis; G6PD deficiency; galactosialidosis; Galloway syndrome; Gardner syndrome; Gaucher disease; Gitelman syndrome; GLUT1 deficiency; glycogen storage disease type 1b; glycogen storage disease type 2; glycogen storage disease type 3; glycogen storage disease type 4; glycogen storage disease type 9a; glycogen storage diseases; GM1-gangliosidosis; Greenberg syndrome; Greig cephalopolysyndactyly syndrome; hair genetic diseases; HANAC syndrome; harlequin type ichtyosis congenita; HDR syndrome; hemochromatosis type 3; hemochromatosis type 4; hemophilia A; hereditary angioedema type 3; hereditary angioedemas; hereditary hemorrhagic telangiectasia; hereditary hypofibrinogenemia; hereditary intraosseous vascular malformation; hereditary leiomyomatosis and renal cell cancer; hereditary neuralgic amyotrophy; hereditary sensory and autonomic neuropathy type; Hermansky-Pudlak disease; HHH syndrome; HHT2; hidrotic ectodermal dysplasia type 1; hidrotic ectodermal dysplasias; HNF4A-associated hyperinsulinism; HNPCC; human immunodeficiency with microcephaly; Huntington disease; hyper-IgD syndrome; hyperinsulinism-hyperammonemia syndrome; hypertrophy of the retinal pigment epithelium; hypochondrogenesis; hypohidrotic ectodermal dysplasia; ICF syndrome; idiopathic congenital intestinal pseudo-obstruction; immunodeficiency with hyper-IgM type 1; immunodeficiency with hyper-IgM type 3; immunodeficiency with hyper-IgM type 4; immunodeficiency with hyper-IgM type 5; inborm errors of thyroid metabolism; infantile visceral myopathy; infantile X-linked spinal muscular atrophy; intrahepatic cholestasis of pregnancy; IPEX syndrome; IRAK4 deficiency; isolated congenital asplenia; Jeune syndrome Imag; Johanson-Blizzard syndrome; Joubert syndrome; JP-HHT syndrome; juvenile hemochromatosis; juvenile hyalin fibromatosis; juvenile nephronophthisis; Kabuki mask syndrome; Kallmann syndromes; Kartagener syndrome; KCNJ11-associated hyperinsulinism; Kearns-Sayre syndrome; Kostmann disease; Kozlowski type of spondylometaphyseal dysplasia; Krabbe disease; LADD syndrome; late infantile-onset neuronal ceroid lipofuscinosis; LCK deficiency; LDHCP syndrome; Legius syndrome; Leigh syndrome; lethal congenital contracture syndrome 2; lethal congenital contracture syndromes; lethal contractural syndrome type 3; lethal neonatal CPT deficiency type 2; lethal osteosclerotic bone dysplasia; LIG4 syndrome; lissencephaly type 1 Imag; lissencephaly type 3; Loeys-Dietz syndrome; low phospholipid-associated cholelithiasis; lysinuric protein intolerance; Maffucci syndrome; Majeed syndrome; mannose-binding protein deficiency; Marfan disease; Marshall syndrome; MASA syndrome; MCAD deficiency; McCune-Albright syndrome; MCKD2; Meckel syndrome; Meesmann corneal dystrophy; megacystis-microcolon-intestinal hypoperistalsis; megaloblastic anemia type 1; MEHMO; MELAS; Melnick-Needles syndrome; MEN2s; Menkes disease; metachromatic leukodystrophies; methylmalonic acidurias; methylvalonic aciduria; microcoria-congenital nephrosis syndrome; microvillous atrophy; mitochondrial neurogastrointestinal encephalomyopathy; monilethrix; monosomy X; mosaic trisomy 9 syndrome; Mowat-Wilson syndrome; mucolipidosis type 2; mucolipidosis type Ma; mucolipidosis type IV; mucopolysaccharidoses; mucopolysaccharidosis type 3A; mucopolysaccharidosis type 3C; mucopolysaccharidosis type 4B; multiminicore disease; multiple acyl-CoA dehydrogenation deficiency; multiple cutaneous and mucosal venous malformations; multiple endocrine neoplasia type 1; multiple sulfatase deficiency; NAIC; nail-patella syndrome; nemaline myopathies; neonatal diabetes mellitus; neonatal surfactant deficiency; nephronophtisis; Netherton disease; neurofibromatoses; neurofibromatosis type 1; Niemann-Pick disease type A; Niemann-Pick disease type B; Niemann-Pick disease type C; NKX2E; Noonan syndrome; North American Indian childhood cirrhosis; NROB1 duplication-associated DSD; ocular genetic diseases; oculo-auricular syndrome; OLEDAID; oligomeganephronia; oligomeganephronic renal hypolasia; Ollier disease; Opitz-Kaveggia syndrome; orofaciodigital syndrome type 1; orofaciodigital syndrome type 2; osseous Paget disease; otopalatodigital syndrome type 2; OXPHOS diseases; palmoplantar hyperkeratosis; panlobar nephroblastomatosis; Parkes-Weber syndrome; Parkinson disease; partial deletion of 21q22.2-q22.3; Pearson syndrome; Pelizaeus-Merzbacher disease; Pendred syndrome; pentalogy of Cantrell; peroxisomal acyl-CoA-oxidase deficiency; Peutz-Jeghers syndrome; Pfeiffer syndrome; Pierson syndrome; pigmented nodular adrenocortical disease; pipecolic acidemia; Pitt-Hopkins syndrome; plasmalogens deficiency; pleuropulmonary blastoma and cystic nephroma; polycystic lipomembranous osteodysplasia; porphyrias; premature ovarian failure; primary erythermalgia; primary hemochromatoses; primary hyperoxaluria; progressive familial intrahepatic cholestasis; propionic acidemia; pyruvate decarboxylase deficiency; RAPADILTINO syndrome; renal cystinosis; rhabdoid tumor predisposition syndrome; Rieger syndrome; ring chromosome 4; Roberts syndrome; Robinow-Sorauf syndrome; Rothmund-Thomson syndrome; SCID; Saethre-Chotzen syndrome; Sandhoff disease; SC phocomelia syndrome; SCAS; Schinzel phocomelia syndrome; short rib-polydactyly syndrome type 1; short rib-polydactyly syndrome type 4; short-rib polydactyly syndrome type 2; short-rib polydactyly syndrome type 3; Shwachman disease; Shwachman-Diamond disease; sickle cell anemia; Silver-Russell syndrome; Simpson-Golabi-Behmel syndrome; Smith-Lemli-Opitz syndrome; SPG7-associated hereditary spastic paraplegia; spherocytosis; split-hand/foot malformation with long bone deficiencies; spondylocostal dysostosis; sporadic visceral myopathy with inclusion bodies; storage diseases; STRA6-associated syndrome; Tay-Sachs disease; thanatophoric dysplasia; thyroid metabolism diseases; Tourette syndrome; transthyretin-associated amyloidosis; trisomy 13; trisomy 22; trisomy 2p syndrome; tuberous sclerosis; tufting enteropathy; urea cycle diseases; Van Den Ende-Gupta syndrome; Van der Woude syndrome; variegated mosaic aneuploidy syndrome; VLCAD deficiency; von Hippel-Lindau disease; Waardenburg syndrome; WAGR syndrome; Walker-Warburg syndrome; Werner syndrome; Wilson disease; Wolcott-Rallison syndrome; Wolfram syndrome; X-linked agammaglobulinemia; X-linked chronic idiopathic intestinal pseudo-obstruction; X-linked cleft palate with ankyloglossia; X-linked dominant chondrodysplasia punctata; X-linked ectodermal dysplasia; X-linked Emery-Dreifuss muscular dystrophy; X-linked lissencephaly; X-linked lymphoproliferative disease; X-linked visceral heterotaxy; xanthinuria type 1; xanthinuria type 2; xeroderma pigmentosum; XPV; and Zellweger disease. In some embodiments, the ailment is Duchenne muscular dystrophy. In some embodiments, the ailment is myotonic dystrophy Type 1 (DM1). In some embodiments, the ailment is blindness or an inherited disease affecting the back of the eye. In some embodiments, the ailment is deafness. In some embodiments, the ailment is progeria. In some embodiments, the ailment is multiple sclerosis. In some embodiments, the ailment is cancer. In some embodiments, the ailment is a lysosomal storage disease, e.g., Hunter syndrome, Hurler syndrome. In some embodiments, the ailment is hypercholesterolemia. In some embodiments, the ailment is Stargardt macular dystrophy. In some embodiments, the ailment is In preferred embodiments, the ailment is cystic fibrosis.

In some embodiments, treating, preventing, or inhibiting an ailment in a subject may comprise contacting a target nucleic acid associated with a particular ailment to a programmable nuclease (e.g., a CasΦ programmable nuclease). In some aspects, the methods of treating, preventing, or inhibiting an ailment may involve removing, modifying, replacing, transposing, or affecting the regulation of a genomic sequence of a patient in need thereof. In some embodiments, the methods of treating, preventing, or inhibiting an ailment may involve modulating gene expression. In some embodiments, the methods of treating, preventing, or inhibiting an ailment may comprise targeting a nucleic acid sequence associated with a pathogen, such as a virus or bacteria, to a programmable nuclease of the present disclosure.

The compositions and methods described herein may be used to treat, prevent, diagnose, or identify a cancer in a subject. In some aspects, the methods may target cells or tissues. In some embodiments, the methods may be applied to subjects, such as humans. As used herein, the term “cancer” refers to a physiological condition that may be characterized by abnormal or unregulated cell growth or activity. In some cases, cancer may involve the spread of the cells exhibiting abnormal or unregulated growth or activity between various tissues in a subject. In some aspects, cancer may be a genetic condition. Examples of cancers include, but are not limited to Acute Lymphoblastic Leukemia, Acute Myeloid Leukemia, Adrenocortical Carcinoma, Anal Cancer, Astrocytomas, Bile Duct Cancer, Bladder Cancer, Bone Cancer, Brain Cancer, Breast Cancer, Bronchial Cancer, Burkitt Lymphoma, Carcinoma, Cardiac Tumors, Cervical Cancer, Chordoma, Chronic Lymphocytic Leukemia, Chronic Myelogenous Leukemia, Chronic Myeloproliferative Neoplasms, Colon Cancer, Colorectal Cancer, Craniopharyngioma, Cutaneous T-cell lymphoma, Ductal Carcinoma, Embryonal Tumors, Endometrial Cancer, Ependymoma, Esophageal Cancer, Esthesioneuroblastoma, Ewing Sarcoma, Extracranial Germ Cell Tumors, Extragonadal Germ Cell Tumors, Fallopian Tube Cancer, Fibrous Histiocytoma, Gallbladder Cancer, Gastric Cancer, Gastrointestinal Cancer, Gastrointestinal Carcinoid Cancer, Gastrointestinal Stromal Tumors, Gestational Trophoblastic Disease, Hairy Cell Leukemia, Head and Neck Cancer, Heart Tumors, Hepatocellular Cancer, Histiocytosis, Hodgkin Lymphoma, Hypopharyngeal Cancer, Intraocular Melanoma, Islet Cell Tumors, Kaposi Sarcoma, Kidney cancer, Langerhans Cell Histiocytosis, Laryngeal Cancer, Leukemia, Lip and Oral Cavity Cancer, Liver Cancer, Lung Cancer, Lymphoma, Malignant Fibrous Histiocytoma, Melanoma, Merkel Cell Carcinoma, Mesothelioma, Metastatic Squamous Neck Cancer, Midline Tract Carcinoma, Mouth Cancer, Multiple Endocrine Neoplasia Syndromes, Multiple Myeloma, Mycosis Fungoides, Myelodysplastic Syndromes, Myelogenous Leukemia, Myeloid Leukemia, Myeloproliferative Neoplasms, Nasal Cavity and Paranasal Sinus Cancer, Nasopharyngeal Cancer, Neuroblastoma, Non-Hodgkin Lymphoma, Non-Small Cell Lung Cancer, Oral Cancer, Osteosarcoma, Ovarian Cancer, Pancreatic Cancer, Pancreatic Neuroendocrine Tumors, Papillomatosis, Paraganglioma, Paranasal Sinus and Nasal Cavity Cancer, Parathyroid Cancer, Penile Cancer, Pharyngeal Cancer, Pheochromocytoma, Pituitary Tumor, Plasma Cell Neoplasm, Pleuropulmonary Blastoma, Primary Central Nervous System (CNS) Lymphoma, Primary Peritoneal Cancer, Prostate Cancer, Rectal Cancer, Recurrent Cancer, Renal Cell Cancer, Retinoblastoma, Rhabdomyosarcoma, Salivary Gland Cancer, Sezary Syndrome, Skin Cancer, Small Cell Lung Cancer, Small Intestine Cancer, Soft Tissue Sarcoma, Squamous Cell Carcinoma, Squamous Neck Cancer with Occult Primary, Stomach Cancer, T-Cell Lymphoma, Testicular Cancer, Throat Cancer, Thymoma and Thymic Carcinoma, Thyroid Cancer, Tracheobronchial Cancer, Transitional Cell Cancer of the Renal Pelvis and Ureter, Ureter Cancer, Renal Pelvis Cancer, Urethral Cancer, Uterine Cancer, Uterine Sarcoma, Vaginal Cancer, Vascular Tumors, Vulvar Cancer, and Wilms Tumor.

In some cases, a cancer is associated with one or more particular biomarkers. A biomarker is a chemical species or profile that may serve as an indicator of a cellular or organismal state (e.g., the presence or absence of a disease). Non-limiting examples of biomarkers include biomolecules, nucleic acid sequences, proteins, metabolites, nucleic acids, protein modifications. A biomarker may refer to one species or to a plurality of species, such as a cell surface profile.

The methods of the present disclosure (e.g., methods of modifying a target nucleic acid) may comprise targeting a biomarker or a nucleic acid associated with a biomarker with a programmable nuclease of the disclosure (e.g., a CasΦ). In some cases, the biomarker is a gene associated with a cancer. Non-limiting examples of genes associated with cancers include, ABL, AF4/HRX, AKT-2, ALK, ALK/NPM, AML1, AML1/MTG8, APC, ATM, AXIN2, AXL, BAP1, BARD1, BCL-2, BCL-3, BCL-6, BCR/ABL, BLM, BMPR1A, BRCA1, BRCA2, BRIP1, c-MYC, CASR, CDC73, CDH1, CDK4, CDKN1B, CDKN1C, CDKN2A, CEBPA, CHEK2, CTNNA1, DBL, DEK/CAN, DICER1, DIS3L2, E2A/PBX1, EGFR, ENL/HRX, EPCAM, ERG/TLS, ERBB, ERBB-2, ETS-1, EWS/FLI-1, FH, FLCN, FMS, FOS, FPS, GATA2, GLI, GPGSP, GREM1, HER2/neu, HOX11, HOXB13, HST, IL-3, INT-2, JUN, KIT, KS3, K-SAM, LBC, LCK, LMO1, LMO2, L-MYC, LYL-1, LYT-10, LYT-10/Cα1, MAS, MAX, MDM-2, MEN1, MET, MITF, MLH1, MLL, MOS, MSH1, MSH2, MSH3, MSH6, MTG8/AML1, MUTYH, MYB, MYH11/CBFB, NBN, NEU, NF1, NF2, N-MYC, NTHL1, OST, PALB2, PAX-5, PBX1/E2A, PDGFRA, PHOX2B, PIM-1, PMS2, POLD1, POLE, POT1, PRAD-1, PRKAR1A, PTCH1, PTEN, RAD50, RAD51C, RAD51D, RAF, RAR/PML, RAS-H, RAS-K, RAS-N, RB1, RECQL4, REL/NRG, RET, RHOM1, RHOM2, ROS, RUNX1, SDHA, SDHAF, SDHB, SDHC, SDHD, SET/CAN, SIS, SKI, SMAD4, SMARCA4, SMARCB1, SMARCE1, SRC, STK11, SUFU, TAL1, TAL2, TAN-1, TIAM1, TERC, TERT, TMEM127, TP53, TSC1, TSC2, TRK, VHL, WRN, and WT1. In some cases, a gene biomarker for cancer will carry one or more mutations. In some cases, a gene biomarker for a cancer will be upregulated or downregulated relative to a patient or sample that does not have the cancer.

The compositions and methods described herein may be suitable for autologous or allogeneic treatment, as well as ex vivo cell-based treatments.

The compositions and methods described herein may be used to treat, prevent, diagnose, or identify an infection in a subject. In some embodiments, the subject is an animal (e.g., a mammal, such as a human). In some embodiments, the subject is a plant (e.g., a crop).

In some aspects, the disclosure provides the programmable CasΦ nucleases and compositions described herein for use in a method of treatment. In some embodiments, the disclosure provides the CasΦ programmable nucleases and compositions described herein for use in a method of treating an ailment recited above.

In some aspects, the disclosure provides the programmable CasΦ nucleases and compositions described herein for use as a medicament.

### Methods of Detecting a Target Nucleic Acid

The present disclosure provides methods and compositions, which enable target nucleic acid detection by programmable nuclease platforms, such as the DNA Endonuclease Targeted CRISPR TransReporter (DETECTR) platform. In some embodiments, the target nucleic acid is a DNA. In some embodiments, the target nucleic acid is a RNA.

A number of reagents are consistent with the compositions and methods disclosed herein. The reagents described herein may be used for nicking target nucleic acids and for detection of target nucleic acids. The reagents disclosed herein can include programmable nucleases, guide nucleic acids, target nucleic acids, and buffers. As described herein, target nucleic acid comprising DNA or RNA may be modified or detected (e.g., the target nucleic acid hybridizes to the guide nucleic) using a programmable nuclease (e.g., a CasΦ as disclosed herein) and other reagents disclosed herein. As described herein, target nucleic acids comprising DNA may be an amplicon of a nucleic acid of interest and the amplicon can be detected using a programmable nuclease (e.g., a CasΦ as disclosed herein) and other reagents disclosed herein. Additionally, detection of multiple target nucleic acids is possible using two or more programmable nickases or a programmable nickase with a non-nickase programmable nuclease complexed to guide nucleic acids that target the multiple target nucleic acids, wherein the programmable nucleases exhibit different sequence-independent cleavage of the nucleic acid of a reporter (e.g., cleavage of an RNA reporter by a first programmable nuclease and cleavage of a DNA reporter by a second programmable nuclease).

In some embodiments, target nucleic acid from a sample is amplified before assaying for cleavage of reporters. Target DNA can be amplified by PCR or isothermal amplification techniques. DNA amplification methods that are compatible with the DETECTR technology can be used for programmable nucleases disclosed herein. For example, ssDNA can be amplified. Amplification of ssDNA instead of dsDNA can enable PAM-independent detection of nucleic acids by proteins with PAM requirements for dsDNA-activated trans-cleavage.

Certain programmable nucleases (e.g., a CasΦ as disclosed herein) of the disclosure can exhibit indiscriminate trans-cleavage of ssDNA, enabling their use for detection of DNA in samples. In some embodiments, target ssDNA are generated from many nucleic acid templates (RNA, ss/dsDNA) in order to achieve cleavage of the FQ reporter in the DETECTR platform. Certain programmable nucleases can be activated by ssDNA, upon which they can exhibit trans-cleavage of ssDNA and can, thereby, be used to cleave ssDNA FQ reporter molecules in the DETECTR system. These programmable nucleases can target ssDNA present in the sample, or generated and/or amplified from any number of nucleic acid templates (RNA, ssDNA, or dsDNA).

The compositions, kits and methods disclosed herein may be implemented in methods of assaying for a target nucleic acid. In some embodiments, a method of assaying for a target nucleic acid in a sample, comprises: contacting the sample to a complex comprising a guide nucleic acid comprising a segment that is reverse complementary to a segment of the target nucleic acid and a programmable nuclease (e.g., a CasΦ as disclosed herein) of the disclosure that exhibits sequence independent cleavage upon forming a complex comprising the segment of the guide nucleic acid binding to the segment of the target nucleic acid, wherein the sample comprises at least one nucleic acid comprising at least 50% sequence identity to the segment of the target nucleic acid; and assaying for cleavage of at least one reporter nucleic acids of a population of reporter nucleic acids, wherein the cleavage indicates a presence of the target nucleic acid in the sample and wherein absence of the cleavage indicates an absence of the target nucleic acid in the sample.

The target nucleic acid can be from 0.05% to 20% of total nucleic acids in the sample. Sometimes, the target nucleic acid is from 0.1% to 10% of the total nucleic acids in the sample. The target nucleic acid, in some cases, is from 0.1% to 5% of the total nucleic acids in the sample. Often, a sample comprises the segment of the target nucleic acid and at least one nucleic acid comprising less than 100% sequence identity to the segment of the target nucleic acid but no less than 50% sequence identity to the segment of the target nucleic acid. For example, the segment of the target nucleic acid comprises a mutation as compared to at least one nucleic acid comprising less than 100% sequence identity to the segment of the target nucleic acid but no less than 50% sequence identity to the segment of the target nucleic acid. Often, the segment of the target nucleic acid comprises a single nucleotide mutation as compared to at least one nucleic acid comprising less than 100% sequence identity to the segment of the target nucleic acid but no less than 50% sequence identity to the segment of the target nucleic acid.

The concentrations of the various reagents in the programmable nuclease DETECTR reaction mix can vary depending on the particular scale of the reaction. For example, the final concentration of the programmable nuclease can vary from 1 μM to 1 nM, from 1 μM to 10 μM, from 10 μM to 100 μM, from 100 μM to 1 nM, from 1 nM to 10 nM, from 10 nM to 20 nM, from 20 nM to 30 nM, from 30 nM to 40 nM, from 40 nM to 50 nM, from 50 nM to 60 nM, from 60 nM to 70 nM, from 70 nM to 80 nM, from 80 nM to 90 nM, from 90 nM to 100 nM, from 100 nM to 200 nM, from 200 nM to 300 nM, from 300 nM to 400 nM, from 400 nM to 500 nM, from 500 nM to 600 nM, from 600 nM to 700 nM, from 700 nM to 800 nM, from 800 nM to 900 nM, from 900 nM to 1000 nM. The final concentration of the sgRNA complementary to the target nucleic acid can be from 1 μM to 1 nM, from 1 μM to 10 μM, from 10 μM to 100 μM, from 100 μM to 1 nM, from 1 nM to 10 nM, from 10 nM to 20 nM, from 20 nM to 30 nM, from 30 nM to 40 nM, from 40 nM to 50 nM, from 50 nM to 60 nM, from 60 nM to 70 nM, from 70 nM to 80 nM, from 80 nM to 90 nM, from 90 nM to 100 nM, from 100 nM to 200 nM, from 200 nM to 300 nM, from 300 nM to 400 nM, from 400 nM to 500 nM, from 500 nM to 600 nM, from 600 nM to 700 nM, from 700 nM to 800 nM, from 800 nM to 900 nM, from 900 nM to 1000 nM. The concentration of the ssDNA-FQ reporter can be from 1 μM to 1 nM, from 1 μM to 10 μM, from 10 μM to 100 μM, from 100 μM to 1 nM, from 1 nM to 10 nM, from 10 nM to 20 nM, from 20 nM to 30 nM, from 30 nM to 40 nM, from 40 nM to 50 nM, from 50 nM to 60 nM, from 60 nM to 70 nM, from 70 nM to 80 nM, from 80 nM to 90 nM, from 90 nM to 100 nM, from 100 nM to 200 nM, from 200 nM to 300 nM, from 300 nM to 400 nM, from 400 nM to 500 nM, from 500 nM to 600 nM, from 600 nM to 700 nM, from 700 nM to 800 nM, from 800 nM to 900 nM, from 900 nM to 1000 nM.

An example of a DETECTR reaction comprises, consists, or consists essentially of a final concentration of 100 nM CasΦ polypeptide or variant thereof, 125 nM sgRNA, and 50 nM ssDNA-FQ reporter in a total reaction volume of 20 μL. Reactions are incubated in a fluorescence plate reader (Tecan Infinite Pro 200 M Plex) for 2 hours at 37° C. with fluorescence measurements taken every 30 seconds (e.g., λex: 485 nm; λem: 535 nm). The fluorescence wavelength detected can vary depending on the reporter molecule.

Described herein are reagents comprising a single stranded reporter nucleic acid comprising a detection moiety, wherein the reporter nucleic acid (e.g., the ssDNA-FQ reporter described above) is capable of being cleaved by the programmable nuclease, upon generation and amplification of ssDNA from a nucleic acid template using the methods disclosed herein, thereby generating a first detectable signal.

The methods disclosed herein, thus, include generation and amplification of ssDNA from a target nucleic acid template (e.g., cDNA, ssDNA, or dsDNA) of interest in a sample, incubation of the ssDNA with an ssDNA activated programmable nuclease leading to indiscriminate, PAM-independent cleavage of reporter nucleic acids (also referred to as ssDNA-FQ reporters) to generate a detectable signal, and quantification of the detectable signal to detect a target nucleic acid sequence of interest.

### Reporters

Described herein are reagents comprising a reporter. The reporter can comprise a single stranded nucleic acid and a detection moiety (e.g., a labeled single stranded DNA reporter), wherein the nucleic acid is capable of being cleaved by the activated programmable nuclease (e.g., a CasΦ as disclosed herein), releasing the detection moiety, and, generating a detectable signal. As used herein, “reporter” is used interchangeably with “reporter nucleic acid” or “reporter molecule”. The programmable nucleases disclosed herein, activated upon hybridization of a guide RNA to a target nucleic acid, can cleave the reporter. Cleaving the “reporter” may be referred to herein as cleaving the “reporter nucleic acid,” the “reporter molecule,” or the “nucleic acid of the reporter.”

A major advantage of the compositions and methods disclosed herein can be the design of excess reporters to total nucleic acids in an unamplified or an amplified sample, not including the nucleic acid of the reporter. Total nucleic acids can include the target nucleic acids and non-target nucleic acids, not including the nucleic acid of the reporter. The non-target nucleic acids can be from the original sample, either lysed or unlysed. The non-target nucleic acids can also be byproducts of amplification. Thus, the non-target nucleic acids can include both non-target nucleic acids from the original sample, lysed or unlysed, and from an amplified sample. The presence of a large amount of non-target nucleic acids, an activated programmable nuclease (e.g., a CasΦ as disclosed herein) may be inhibited in its ability to bind and cleave the reporter sequences. This is because the activated programmable nuclease collaterally cleaves any nucleic acids. If total nucleic acids are in present in large amounts, they may outcompete reporters for the programmable nucleases. The compositions and methods disclosed herein are designed to have an excess of reporter to total nucleic acids, such that the detectable signals from DETECTR reactions are particularly superior. In some embodiments, the reporter can be present in at least 1.5 fold, at least 2 fold, at least 3 fold, at least 4 fold, at least 5 fold, at least 6 fold, at least 7 fold, at least 8 fold, at least 9 fold, at least 10 fold, at least 11 fold, at least 12 fold, at least 13 fold, at least 14 fold, at least 15 fold, at least 16 fold, at least 17 fold, at least 18 fold, at least 19 fold, at least 20 fold, at least 30 fold, at least 40 fold, at least 50 fold, at least 60 fold, at least 70 fold, at least 80 fold, at least 90 fold, at least 100 fold, from 1.5 fold to 100 fold, from 2 fold to 10 fold, from 10 fold to 20 fold, from 20 fold to 30 fold, from 30 fold to 40 fold, from 40 fold to 50 fold, from 50 fold to 60 fold, from 60 fold to 70 fold, from 70 fold to 80 fold, from 80 fold to 90 fold, from 90 fold to 100 fold, from 1.5 fold to 10 fold, from 1.5 fold to 20 fold, from 10 fold to 40 fold, from 20 fold to 60 fold, or from 10 fold to 80 fold excess of total nucleic acids.

Another significant advantage of the compositions and methods disclosed herein can be the design of an excess volume comprising the guide nucleic acid, the programmable nuclease (e.g., a CasΦ as disclosed herein), and the reporter, which contacts a smaller volume comprising the sample with the target nucleic acid of interest. The smaller volume comprising the sample can be unlysed sample, lysed sample, or lysed sample which has undergone any combination of reverse transcription, amplification, and in vitro transcription. The presence of various reagents in a crude, non-lysed sample, a lysed sample, or a lysed and amplified sample, such as buffer, magnesium sulfate, salts, the pH, a reducing agent, primers, dNTPs, NTPs, cellular lysates, non-target nucleic acids, primers, or other components, can inhibit the ability of the programmable nuclease to become activated or to find and cleave the nucleic acid of the reporter. This may be due to nucleic acids that are not the reporter outcompeting the nucleic acid of the reporter, for the programmable nuclease. Alternatively, various reagents in the sample may simply inhibit the activity of the programmable nuclease. Thus, the compositions and methods provided herein for contacting an excess volume comprising the guide nucleic acid, the programmable nuclease, and the reporter to a smaller volume comprising the sample with the target nucleic acid of interest provides for superior detection of the target nucleic acid by ensuring that the programmable nuclease is able to find and cleaves the nucleic acid of the reporter. In some embodiments, the volume comprising the guide nucleic acid, the programmable nuclease, and the reporter (can be referred to as “a second volume”) is 4-fold greater than a volume comprising the sample (can be referred to as “a first volume”). In some embodiments, the volume comprising the guide nucleic acid, the programmable nuclease, and the reporter (can be referred to as “a second volume”) is at least 1.5 fold, at least 2 fold, at least 3 fold, at least 4 fold, at least 5 fold, at least 6 fold, at least 7 fold, at least 8 fold, at least 9 fold, at least 10 fold, at least 11 fold, at least 12 fold, at least 13 fold, at least 14 fold, at least 15 fold, at least 16 fold, at least 17 fold, at least 18 fold, at least 19 fold, at least 20 fold, at least 30 fold, at least 40 fold, at least 50 fold, at least 60 fold, at least 70 fold, at least 80 fold, at least 90 fold, at least 100 fold, from 1.5 fold to 100 fold, from 2 fold to 10 fold, from 10 fold to 20 fold, from 20 fold to 30 fold, from 30 fold to 40 fold, from 40 fold to 50 fold, from 50 fold to 60 fold, from 60 fold to 70 fold, from 70 fold to 80 fold, from 80 fold to 90 fold, from 90 fold to 100 fold, from 1.5 fold to 10 fold, from 1.5 fold to 20 fold, from 10 fold to 40 fold, from 20 fold to 60 fold, or from 10 fold to 80 fold greater than a volume comprising the sample (can be referred to as “a first volume”). In some embodiments, the volume comprising the sample is at least 0.5 μL, at least 1 μL, at least at least 1 μL, at least 2 μL, at least 3 μL, at least 4 μL, at least 5 μL, at least 6 μL, at least 7 μL, at least 8 μL, at least 9 μL, at least 10 μL, at least 11 μL, at least 12 μL, at least 13 μL, at least 14 μL, at least 15 μL, at least 16 μL, at least 17 μL, at least 18 μL, at least 19 μL, at least 20 μL, at least 25 μL, at least 30 μL, at least 35 μL, at least 40 μL, at least 45 μL, at least 50 μL, at least 55 μL, at least 60 μL, at least 65 μL, at least 70 μL, at least 75 μL, at least 80 μL, at least 85 μL, at least 90 μL, at least 95 μL, at least 100 μL, from 0.5 μL to 5 μL μL, from 5 μL to 10 μL, from 10 μL to 15 μL, from 15 μL to 20 μL, from 20 μL to 25 μL, from 25 μL to 30 μL, from 30 μL to 35 μL, from 35 μL to 40 μL, from 40 μL to 45 μL, from 45 μL to 50 μL, from 10 μL to 20 μL, from 5 μL to 20 μL, from 1 μL to 40 μL, from 2 μL to 10 μL, or from 1 μL to 10 μL. In some embodiments, the volume comprising the programmable nuclease, the guide nucleic acid, and the reporter is at least 10 μL, at least 11 μL, at least 12 μL, at least 13 μL, at least 14 μL, at least 15 μL, at least 16 μL, at least 17 μL, at least 18 μL, at least 19 μL, at least 20 μL, at least 21 μL, at least 22 μL, at least 23 μL, at least 24 μL, at least 25 μL, at least 26 μL, at least 27 μL, at least 28 μL, at least 29 μL, at least 30 μL, at least 40 μL, at least 50 μL, at least 60 μL, at least 70 μL, at least 80 μL, at least 90 μL, at least 100 μL, at least 150 μL, at least 200 μL, at least 250 μL, at least 300 μL, at least 350 μL, at least 400 μL, at least 450 μL, at least 500 μL, from 10 μL to 15 μL μL, from 15 μL to 20 μL, from 20 μL to 25 μL, from 25 μL to 30 μL, from 30 μL to 35 μL, from 35 μL to 40 μL, from 40 μL to 45 μL, from 45 μL to 50 μL, from 50 μL to 55 μL, from 55 μL to 60 μL, from 60 μL to 65 μL, from 65 μL to 70 μL, from 70 μL to 75 μL, from 75 μL to 80 μL, from 80 μL to 85 μL, from 85 μL to 90 μL, from 90 μL to 95 μL, from 95 μL to 100 μL, from 100 μL to 150 μL, from 150 μL to 200 μL, from 200 μL to 250 μL, from 250 μL to 300 μL, from 300 μL to 350 μL, from 350 μL to 400 μL, from 400 μL to 450 μL, from 450 μL to 500 μL, from 10 μL to 20 μL, from 10 μL to 30 μL, from 25 μL to 35 μL, from 10 μL to 40 μL, from 20 μL to 50 μL, from 18 μL to 28 μL, or from 17 μL to 22 μL.

In some cases, the reporter nucleic acid is a single-stranded nucleic acid sequence comprising deoxyribonucleotides. In other cases, the reporter nucleic acid is a single-stranded nucleic acid sequence comprising ribonucleotides. The nucleic acid of a reporter can be a single-stranded nucleic acid sequence comprising at least one deoxyribonucleotide and at least one ribonucleotide. In some cases, the nucleic acid of a reporter is a single-stranded nucleic acid comprising at least one ribonucleotide residue at an internal position that functions as a cleavage site. In some cases, the nucleic acid of a reporter comprises at least 2, 3, 4, 5, 6, 7, 8, 9, or 10 ribonucleotide residues at an internal position. In some cases, the nucleic acid of a reporter comprises from 2 to 10, from 3 to 9, from 4 to 8, or from 5 to 7 ribonucleotide residues at an internal position. Sometimes the ribonucleotide residues are continuous. Alternatively, the ribonucleotide residues are interspersed in between non-ribonucleotide residues. In some cases, the nucleic acid of a reporter has only ribonucleotide residues. In some cases, the nucleic acid of a reporter has only deoxyribonucleotide residues. In some cases, the nucleic acid comprises nucleotides resistant to cleavage by the programmable nuclease described herein. In some cases, the nucleic acid of a reporter comprises synthetic nucleotides. In some cases, the nucleic acid of a reporter comprises at least one ribonucleotide residue and at least one non-ribonucleotide residue. In some cases, the nucleic acid of a reporter is 5-20, 5-15, 5-10, 7-20, 7-15, or 7-10 nucleotides in length. In some cases, the nucleic acid of a reporter is from 3 to 20, from 4 to 10, from 5 to 10, or from 5 to 8 nucleotides in length. In some cases, the nucleic acid of a reporter comprises at least one uracil ribonucleotide. In some cases, the nucleic acid of a reporter comprises at least two uracil ribonucleotides. Sometimes the nucleic acid of a reporter has only uracil ribonucleotides. In some cases, the nucleic acid of a reporter comprises at least one adenine ribonucleotide. In some cases, the nucleic acid of a reporter comprises at least two adenine ribonucleotides. In some cases, the nucleic acid of a reporter has only adenine ribonucleotides. In some cases, the nucleic acid of a reporter comprises at least one cytosine ribonucleotide. In some cases, the nucleic acid of a reporter comprises at least two cytosine ribonucleotides. In some cases, the nucleic acid of a reporter comprises at least one guanine ribonucleotide. In some cases, the nucleic acid of a reporter comprises at least two guanine ribonucleotides. A nucleic acid of a reporter can comprise only unmodified ribonucleotides, only unmodified deoxyribonucleotides, or a combination thereof. In some cases, the nucleic acid of a reporter is from 5 to 12 nucleotides in length. In some cases, the reporter nucleic acid is at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, or at least 30 nucleotides in length. In some cases, the reporter nucleic acid is 2, 3, 4, 5, 6, 7, 8, 9, 10, 11, 12, 13, 14, 15, 16, 17, 18, 19, 20, 21, 22, 23, 24, 25, 26, 27, 28, 29, or 30 nucleotides in length.

The single stranded nucleic acid of a reporter comprises a detection moiety capable of generating a first detectable signal. Sometimes the reporter nucleic acid comprises a protein capable of generating a signal. A signal can be a calorimetric, potentiometric, amperometric, optical (e.g., fluorescent, colorimetric, etc.), or piezo-electric signal. In some cases, a detection moiety is on one side of the cleavage site. Optionally, a quenching moiety is on the other side of the cleavage site. Sometimes the quenching moiety is a fluorescence quenching moiety. In some cases, the quenching moiety is 5′ to the cleavage site and the detection moiety is 3′ to the cleavage site. In some cases, the detection moiety is 5′ to the cleavage site and the quenching moiety is 3′ to the cleavage site. Sometimes the quenching moiety is at the 5′ terminus of the nucleic acid of a reporter. Sometimes the detection moiety is at the 3′ terminus of the nucleic acid of a reporter. In some cases, the detection moiety is at the 5′ terminus of the nucleic acid of a reporter. In some cases, the quenching moiety is at the 3′ terminus of the nucleic acid of reporter. In some cases, the single-stranded nucleic acid of a reporter is at least one population of the single-stranded nucleic acid capable of generating a first detectable signal. In some cases, the single-stranded nucleic acid of a reporter is a population of the single stranded nucleic acid capable of generating a first detectable signal. Optionally, there is more than one population of single-stranded nucleic acid of a reporter. In some cases, there are 2, 3, 4, 5, 6, 7, 8, 9, 10, 11, 12, 13, 14, 15, 20, 30, 40, 50, or greater than 50, or any number spanned by the range of this list of different populations of single-stranded nucleic acids of a reporter capable of generating a detectable signal. In some cases, there are from 2 to 50, from 3 to 40, from 4 to 30, from 5 to 20, or from 6 to 10 different populations of single-stranded nucleic acids of a reporter capable of generating a detectable signal.

TABLE 3
Examples of_Single_Stranded Nucleic Acids in a Reporter

| 5′ Detection Moiety* | Sequence (SEQ ID NO) | 3′ Quencher* |
|---|---|---|
| /56-FAM/ | TTATTATT (SEQ ID NO: 95) | /3IABkFQ/ |
| /56-FAM/ | TTATTATT (SEQ ID NO: 95) | /3IABkFQ/ |
| /5IRD700/ | TTATTATT (SEQ ID NO: 95) | /3IRQC1N/ |
| /5TYE665/ | TTATTATT (SEQ ID NO: 95) | /3IAbRQSp/ |
| /5Alex594N/ | TTATTATT (SEQ ID NO: 95) | /3IAbRQSp/ |
| /5ATTO633N/ | TTATTATT (SEQ ID NO: 95) | /3IAbRQSp/ |
| /56-FAM/ | TTTTTT (SEQ ID NO: 96) | /3IABkFQ/ |
| /56-FAM/ | TTTTTTTT (SEQ ID NO: 97) | /3IABkFQ/ |
| /56-FAM/ | TTTTTTTTTT (SEQ ID NO: 98) | /3IABkFQ/ |
| /56-FAM/ | TTTTTTTTTTTT (SEQ ID NO: 99) | /3IABkFQ/ |
| /56-FAM/ | TTTTTTTTTTTTTT (SEQ ID NO: 100) | /3IABkFQ/ |
| /56-FAM/ | AAAAAA (SEQ ID NO: 101) | /3IABkFQ/ |
| /56-FAM/ | CCCCCC (SEQ ID NO: 102) | /3IABkFQ/ |
| /56-FAM/ | GGGGGG (SEQ ID NO: 103) | /3IABkFQ/ |
| /56-FAM/ | TTATTATT (SEQ ID NO: 104) | /3IABkFQ/ |

*This Table refers to the detection moiety and quencher moiety as their tradenames and their source is identified. However, alternatives, generics, or non-tradename moieties with similar function from other sources can also be used.
/56-FAM/: 5′ 6-Fluorescein (Integrated DNA Technologies)
/3IABkFQ/: 3′ Iowa Black FQ (Integrated DNA Technologies)
/5IRD700/: 5′ IRDye 700 (Integrated DNA Technologies)
/5TYE665/: 5′ TYE 665 (Integrated DNA Technologies)
/5Alex594N/: 5′ Alexa Fluor 594 (NHS Ester)(Integrated DNA Technologies)
/5ATTO633N/: 5′ ATTO TM 633 (NHS Ester)(Integrated DNA Technologies)
/3IRQCIN/: 3′ IRDye QC-1 Quencher (Li-Cor)
/3IAbRQSp/: 3′ Iowa Black RQ (Integrated DNA Technologies)

A detection moiety can be an infrared fluorophore. A detection moiety can be a fluorophore that emits fluorescence in the range of from 500 nm and 720 nm. A detection moiety can be a fluorophore that emits fluorescence in the range of from 500 nm and 720 nm. In some cases, the detection moiety emits fluorescence at a wavelength of 700 nm or higher. In other cases, the detection moiety emits fluorescence at about 660 nm or about 670 nm. In some cases, the detection moiety emits fluorescence in the range of from 500 to 520, 500 to 540, 500 to 590, 590 to 600, 600 to 610, 610 to 620, 620 to 630, 630 to 640, 640 to 650, 650 to 660, 660 to 670, 670 to 680, 690 to 690, 690 to 700, 700 to 710, 710 to 720, or 720 to 730 nm. In some cases, the detection moiety emits fluorescence in the range from 450 nm to 750 nm, from 500 nm to 650 nm, or from 550 to 650 nm. A detection moiety can be a fluorophore that emits a detectable fluorescence signal in the same range as 6-Fluorescein, IRDye 700, TYE 665, Alex Fluor, or ATTO TM 633 (NHS Ester). A detection moiety can be fluorescein amidite, 6-Fluorescein, IRDye 700, TYE 665, Alex Fluor 594, or ATTO TM 633 (NHS Ester). A detection moiety can be a fluorophore that emits a fluorescence in the same range as 6-Fluorescein (Integrated DNA Technologies), IRDye 700 (Integrated DNA Technologies), TYE 665 (Integrated DNA Technologies), Alex Fluor 594 (Integrated DNA Technologies), or ATTO TM 633 (NHS Ester) (Integrated DNA Technologies). A detection moiety can be fluorescein amidite, 6-Fluorescein (Integrated DNA Technologies), IRDye 700 (Integrated DNA Technologies), TYE 665 (Integrated DNA Technologies), Alex Fluor 594 (Integrated DNA Technologies), or ATTO TM 633 (NHS Ester) (Integrated DNA Technologies). Any of the detection moieties described herein can be from any commercially available source, can be an alternative with a similar function, a generic, or a non-tradename of the detection moieties listed.

A detection moiety can be chosen for use based on the type of sample to be tested. For example, a detection moiety that is an infrared fluorophore is used with a urine sample. As another example, SEQ ID NO: 87 with a fluorophore that emits a fluorescence around 520 nm is used for testing in non-urine samples, and SEQ ID NO: 94 with a fluorophore that emits a fluorescence around 700 nm is used for testing in urine samples.

A quenching moiety can be chosen based on its ability to quench the detection moiety. A quenching moiety can be a non-fluorescent fluorescence quencher. A quenching moiety can quench a detection moiety that emits fluorescence in the range of from 500 nm and 720 nm. A quenching moiety can quench a detection moiety that emits fluorescence in the range of from 500 nm and 720 nm. In some cases, the quenching moiety quenches a detection moiety that emits fluorescence at a wavelength of 700 nm or higher. In other cases, the quenching moiety quenches a detection moiety that emits fluorescence at about 660 nm or about 670 nm. In some cases, the quenching moiety quenches a detection moiety that emits fluorescence in the range of from 500 to 520, 500 to 540, 500 to 590, 590 to 600, 600 to 610, 610 to 620, 620 to 630, 630 to 640, 640 to 650, 650 to 660, 660 to 670, 670 to 680, 690 to 690, 690 to 700, 700 to 710, 710 to 720, or 720 to 730 nm. In some cases, the quenching moiety quenches a detection moiety that emits fluorescence in the range from 450 nm to 750 nm, from 500 nm to 650 nm, or from 550 to 650 nm. A quenching moiety can quench fluorescein amidite, 6-Fluorescein, IRDye 700, TYE 665, Alex Fluor 594, or ATTO TM 633 (NHS Ester). A quenching moiety can be Iowa Black RQ, Iowa Black FQ or IRDye QC-1 Quencher. A quenching moiety can quench fluorescein amidite, 6-Fluorescein (Integrated DNA Technologies), IRDye 700 (Integrated DNA Technologies), TYE 665 (Integrated DNA Technologies), Alex Fluor 594 (Integrated DNA Technologies), or ATTO TM 633 (NHS Ester) (Integrated DNA Technologies). A quenching moiety can be Iowa Black RQ (Integrated DNA Technologies), Iowa Black FQ (Integrated DNA Technologies) or IRDye QC-1 Quencher (LiCor). Any of the quenching moieties described herein can be from any commercially available source, can be an alternative with a similar function, a generic, or a non-tradename of the quenching moieties listed.

The generation of the detectable signal from the release of the detection moiety indicates that cleavage by the programmable nucleases has occurred and that the sample contains the target nucleic acid. In some cases, the detection moiety comprises a fluorescent dye. Sometimes the detection moiety comprises a fluorescence resonance energy transfer (FRET) pair. In some cases, the detection moiety comprises an infrared (IR) dye. In some cases, the detection moiety comprises an ultraviolet (UV) dye. Alternatively or in combination, the detection moiety comprises a polypeptide. Sometimes the detection moiety comprises a biotin. Sometimes the detection moiety comprises at least one of avidin or streptavidin. In some instances, the detection moiety comprises a polysaccharide, a polymer, or a nanoparticle. In some instances, the detection moiety comprises a gold nanoparticle or a latex nanoparticle.

A detection moiety can be any moiety capable of generating a calorimetric, potentiometric, amperometric, optical (e.g., fluorescent, colorimetric, etc.), or piezo-electric signal. A nucleic acid of a reporter, sometimes, is protein-nucleic acid that is capable of generating a calorimetric, potentiometric, amperometric, optical (e.g., fluorescent, colorimetric, etc.), or piezo-electric signal upon cleavage of the nucleic acid. Often a calorimetric signal is heat produced after cleavage of the nucleic acids of a reporter. Sometimes, a calorimetric signal is heat absorbed after cleavage of the nucleic acids of a reporter. A potentiometric signal, for example, is electrical potential produced after cleavage of the nucleic acids of a reporter. An amperometric signal can be movement of electrons produced after the cleavage of nucleic acid of a reporter. Often, the signal is an optical signal, such as a colorimetric signal or a fluorescence signal. An optical signal is, for example, a light output produced after the cleavage of the nucleic acids of a reporter. Sometimes, an optical signal is a change in light absorbance between before and after the cleavage of nucleic acids of a reporter. Often, a piezo-electric signal is a change in mass between before and after the cleavage of the nucleic acid of a reporter.

The detectable signal can be a colorimetric signal or a signal visible by eye. In some instances, the detectable signal can be fluorescent, electrical, chemical, electrochemical, or magnetic. In some cases, the first detection signal can be generated by binding of the detection moiety to the capture molecule in the detection region, where the first detection signal indicates that the sample contained the target nucleic acid. Sometimes the system can be capable of detecting more than one type of target nucleic acid, wherein the system comprises more than one type of guide nucleic acid and more than one type of reporter nucleic acid. In some cases, the detectable signal can be generated directly by the cleavage event. Alternatively or in combination, the detectable signal can be generated indirectly by the signal event. Sometimes the detectable signal is not a fluorescent signal. In some instances, the detectable signal can be a colorimetric or color-based signal. In some cases, the detected target nucleic acid can be identified based on its spatial location on the detection region of the support medium. In some cases, the second detectable signal can be generated in a spatially distinct location than the first generated signal.

Often, the protein-nucleic acid is an enzyme-nucleic acid. The enzyme may be sterically hindered when present as in the enzyme-nucleic acid, but then functional upon cleavage from the nucleic acid. Often, the enzyme is an enzyme that produces a reaction with a substrate. An enzyme can be invertase. Often, the substrate of invertase is sucrose. A DNS reagent produces a colorimetric change when invertase converts sucrose to glucose. In some cases, it is preferred that the nucleic acid (e.g., DNA) and invertase are conjugated using a heterobifunctional linker via sulfo-SMCC chemistry. Sometimes the protein-nucleic acid is a substrate-nucleic acid. Often the substrate is a substrate that produces a reaction with an enzyme.

A protein-nucleic acid may be attached to a solid support. The solid support, for example, is a surface. A surface can be an electrode. Sometimes the solid support is a bead. Often the bead is a magnetic bead. Upon cleavage, the protein is liberated from the solid and interacts with other mixtures. For example, the protein is an enzyme, and upon cleavage of the nucleic acid of the enzyme-nucleic acid, the enzyme flows through a chamber into a mixture comprising the substrate. When the enzyme meets the enzyme substrate, a reaction occurs, such as a colorimetric reaction, which is then detected. As another example, the protein is an enzyme substrate, and upon cleavage of the nucleic acid of the enzyme substrate-nucleic acid, the enzyme flows through a chamber into a mixture comprising the enzyme. When the enzyme substrate meets the enzyme, a reaction occurs, such as a calorimetric reaction, which is then detected.

Often, the signal is a colorimetric signal or a signal visible by eye. In some instances, the signal is fluorescent, electrical, chemical, electrochemical, or magnetic. A signal can be a calorimetric, potentiometric, amperometric, optical (e.g., fluorescent, colorimetric, etc.), or piezo-electric signal. In some cases, the detectable signal is a colorimetric signal or a signal visible by eye. In some instances, the detectable signal is fluorescent, electrical, chemical, electrochemical, or magnetic. In some cases, the first detection signal is generated by binding of the detection moiety to the capture molecule in the detection region, where the first detection signal indicates that the sample contained the target nucleic acid. Sometimes the system is capable of detecting more than one type of target nucleic acid, wherein the system comprises more than one type of guide nucleic acid and more than one type of nucleic acid of a reporter. In some cases, the detectable signal is generated directly by the cleavage event. Alternatively or in combination, the detectable signal is generated indirectly by the signal event. Sometimes the detectable signal is not a fluorescent signal. In some instances, the detectable signal is a colorimetric or color-based signal. In some cases, the detected target nucleic acid is identified based on its spatial location on the detection region of the support medium. In some cases, the second detectable signal is generated in a spatially distinct location than the first generated signal.

In some cases, the threshold of detection, for a subject method of detecting a single stranded target nucleic acid in a sample, is less than or equal to 10 nM. The term “threshold of detection” is used herein to describe the minimal amount of target nucleic acid that must be present in a sample in order for detection to occur. For example, when a threshold of detection is 10 nM, then a signal can be detected when a target nucleic acid is present in the sample at a concentration of 10 nM or more. In some cases, the threshold of detection is less than or equal to 5 nM, 1 nM, 0.5 nM, 0.1 nM, 0.05 nM, 0.01 nM, 0.005 nM, 0.001 nM, 0.0005 nM, 0.0001 nM, 0.00005 nM, 0.00001 nM, 10 μM, 1 μM, 500 fM, 250 fM, 100 fM, 50 fM, 10 fM, 5 fM, 1 fM, 500 attomole (aM), 100 aM, 50 aM, 10 aM, or 1 aM. In some cases, the threshold of detection is in a range of from 1 aM to 1 nM, 1 aM to 500 μM, 1 aM to 200 μM, 1 aM to 100 μM, 1 aM to 10 μM, 1 aM to 1 μM, 1 aM to 500 fM, 1 aM to 100 fM, 1 aM to 1 fM, 1 aM to 500 aM, 1 aM to 100 aM, 1 aM to 50 aM, 1 aM to 10 aM, 10 aM to 1 nM, 10 aM to 500 μM, 10 aM to 200 μM, 10 aM to 100 μM, 10 aM to 10 μM, 10 aM to 1 μM, 10 aM to 500 fM, 10 aM to 100 fM, 10 aM to 1 fM, 10 aM to 500 aM, 10 aM to 100 aM, 10 aM to 50 aM, 100 aM to 1 nM, 100 aM to 500 μM, 100 aM to 200 μM, 100 aM to 100 μM, 100 aM to 10 μM, 100 aM to 1 μM, 100 aM to 500 fM, 100 aM to 100 fM, 100 aM to 1 fM, 100 aM to 500 aM, 500 aM to 1 nM, 500 aM to 500 μM, 500 aM to 200 μM, 500 aM to 100 μM, 500 aM to 10 μM, 500 aM to 1 μM, 500 aM to 500 fM, 500 aM to 100 fM, 500 aM to 1 fM, 1 fM to 1 nM, 1 fM to 500 μM, 1 fM to 200 μM, 1 fM to 100 μM, 1 fM to 10 μM, 1 fM to 1 μM, 10 fM to 1 nM, 10 fM to 500 μM, 10 fM to 200 μM, 10 fM to 100 μM, 10 fM to 10 μM, 10 fM to 1 μM, 500 fM to 1 nM, 500 fM to 500 μM, 500 fM to 200 μM, 500 fM to 100 μM, 500 fM to 10 μM, 500 fM to 1 μM, 800 fM to 1 nM, 800 fM to 500 pM, 800 fM to 200 μM, 800 fM to 100 μM, 800 fM to 10 μM, 800 fM to 1 μM, 1 μM to 1 nM, 1 μM to 500 μM, 1 μM to 200 μM, 1 μM to 100 μM, or 1 μM to 10 μM. In some cases, the threshold of detection in a range of from 800 fM to 100 μM, 1 μM to 10 μM, 10 fM to 500 fM, 10 fM to 50 fM, 50 fM to 100 fM, 100 fM to 250 fM, or 250 fM to 500 fM. In some cases the threshold of detection is in a range of from 2 aM to 100 μM, from 20 aM to 50 μM, from 50 aM to 20 μM, from 200 aM to 5 μM, or from 500 aM to 2 μM. In some cases, the minimum concentration at which a single stranded target nucleic acid is detected in a sample is in a range of from 1 aM to 1 nM, 10 aM to 1 nM, 100 aM to 1 nM, 500 aM to 1 nM, 1 fM to 1 nM, 1 fM to 500 μM, 1 fM to 200 μM, 1 fM to 100 μM, 1 fM to 10 μM, 1 fM to 1 μM, 10 fM to 1 nM, 10 fM to 500 μM, 10 fM to 200 μM, 10 fM to 100 μM, 10 fM to 10 μM, 10 fM to 1 μM, 500 fM to 1 nM, 500 fM to 500 μM, 500 fM to 200 μM, 500 fM to 100 μM, 500 fM to 10 μM, 500 fM to 1 μM, 800 fM to 1 nM, 800 fM to 500 μM, 800 fM to 200 μM, 800 fM to 100 μM, 800 fM to 10 μM, 800 fM to 1 μM, 1 μM to 1 nM, 1 μM to 500 μM, from 1 μM to 200 μM, 1 μM to 100 μM, or 1 μM to 10 μM. In some cases, the minimum concentration at which a single stranded target nucleic acid is detected in a sample is in a range of from 2 aM to 100 μM, from 20 aM to 50 μM, from 50 aM to 20 μM, from 200 aM to 5 μM, or from 500 aM to 2 μM. In some cases, the minimum concentration at which a single stranded target nucleic acid can be detected in a sample is in a range of from 1 aM to 100 μM. In some cases, the minimum concentration at which a single stranded target nucleic acid can be detected in a sample is in a range of from 1 fM to 100 μM. In some cases, the minimum concentration at which a single stranded target nucleic acid can be detected in a sample is in a range of from 10 fM to 100 μM. In some cases, the minimum concentration at which a single stranded target nucleic acid can be detected in a sample is in a range of from 800 fM to 100 μM. In some cases, the minimum concentration at which a single stranded target nucleic acid can be detected in a sample is in a range of from 1 μM to 10 μM. In some cases, the devices, systems, fluidic devices, kits, and methods described herein detect a target single-stranded nucleic acid in a sample comprising a plurality of nucleic acids such as a plurality of non-target nucleic acids, where the target single-stranded nucleic acid is present at a concentration as low as 1 aM, 10 aM, 100 aM, 500 aM, 1 fM, 10 fM, 500 fM, 800 fM, 1 μM, 10 μM, 100 μM, or 1 μM.

In some embodiments, the target nucleic acid is present in the cleavage reaction at a concentration of about 10 nM, about 20 nM, about 30 nM, about 40 nM, about 50 nM, about 60 nM, about 70 nM, about 80 nM, about 90 nM, about 100 nM, about 200 nM, about 300 nM, about 400 nM, about 500 nM, about 600 nM, about 700 nM, about 800 nM, about 900 nM, about 1 μM, about 10 μM, or about 100 μM. In some embodiments, the target nucleic acid is present in the cleavage reaction at a concentration of from 10 nM to 20 nM, from 20 nM to 30 nM, from 30 nM to 40 nM, from 40 nM to 50 nM, from 50 nM to 60 nM, from 60 nM to 70 nM, from 70 nM to 80 nM, from 80 nM to 90 nM, from 90 nM to 100 nM, from 100 nM to 200 nM, from 200 nM to 300 nM, from 300 nM to 400 nM, from 400 nM to 500 nM, from 500 nM to 600 nM, from 600 nM to 700 nM, from 700 nM to 800 nM, from 800 nM to 900 nM, from 900 nM to 1 μM, from 1 μM to 10 μM, from 10 μM to 100 μM, from 10 nM to 100 nM, from 10 nM to 1 μM, from 10 nM to 10 μM, from 10 nM to 100 μM, from 100 nM to 1 μM, from 100 nM to 10 μM, from 100 nM to 100 μM, or from 1 μM to 100 μM. In some embodiments, the target nucleic acid is present in the cleavage reaction at a concentration of from 20 nM to 50 μM, from 50 nM to 20 μM, or from 200 nM to 5 μM.

In some cases, the methods, compositions, reagents, enzymes, and kits described herein may be used to detect a target single-stranded nucleic acid in a sample where the sample is contacted with the reagents for a predetermined length of time sufficient for the trans-cleavage to occur or cleavage reaction to reach completion. In some cases, the devices, systems, fluidic devices, kits, and methods described herein detect a target single-stranded nucleic acid in a sample where the sample is contacted with the reagents for no greater than 60 minutes. Sometimes the sample is contacted with the reagents for no greater than 120 minutes, 110 minutes, 100 minutes, 90 minutes, 80 minutes, 70 minutes, 60 minutes, 55 minutes, 50 minutes, 45 minutes, 40 minutes, 35 minutes, 30 minutes, 25 minutes, 20 minutes, 15 minutes, 10 minutes, 5 minutes, 4 minutes, 3 minutes, 2 minutes, or 1 minute. Sometimes the sample is contacted with the reagents for at least 120 minutes, 110 minutes, 100 minutes, 90 minutes, 80 minutes, 70 minutes, 60 minutes, 55 minutes, 50 minutes, 45 minutes, 40 minutes, 35 minutes, 30 minutes, 25 minutes, 20 minutes, 15 minutes, 10 minutes, or 5 minutes. In some cases, the sample is contacted with the reagents for from 5 minutes to 120 minutes, from 5 minutes to 100 minutes, from 10 minutes to 90 minutes, from 15 minutes to 45 minutes, or from 20 minutes to 35 minutes. In some cases, the devices, systems, fluidic devices, kits, and methods described herein can detect a target nucleic acid in a sample in less than 10 hours, less than 9 hours, less than 8 hours, less than 7 hours, less than 6 hours, less than 5 hours, less than 4 hours, less than 3 hours, less than 2 hours, less than 1 hour, less than 50 minutes, less than 45 minutes, less than 40 minutes, less than 35 minutes, less than 30 minutes, less than 25 minutes, less than 20 minutes, less than 15 minutes, less than 10 minutes, less than 9 minutes, less than 8 minutes, less than 7 minutes, less than 6 minutes, or less than 5 minutes. In some cases, the devices, systems, fluidic devices, kits, and methods described herein can detect a target nucleic acid in a sample in from 5 minutes to 10 hours, from 10 minutes to 8 hours, from 15 minutes to 6 hours, from 20 minutes to 5 hours, from 30 minutes to 2 hours, or from 45 minutes to 1 hour.

When a guide nucleic acid binds to a target nucleic acid, the programmable nuclease's trans-cleavage activity can be initiated, and nucleic acids of a reporter can be cleaved, resulting in the detection of fluorescence. The guide nucleic acid may be a non-naturally occurring guide nucleic acid. A non-naturally occurring guide nucleic acid may comprise an engineered sequence having a repeat and a spacer that hybridizes to a target nucleic acid sequence of interest. A non-naturally occurring guide nucleic acid may be recombinantly expressed or chemically synthesized. Nucleic acid reporters can comprise a detection moiety, wherein the nucleic acid reporter can be cleaved by the activated programmable nuclease, thereby generating a signal. Some methods as described herein can a method of assaying for a target nucleic acid in a sample comprises contacting the sample to a complex comprising a guide nucleic acid comprising a segment that is reverse complementary to a segment of the target nucleic acid and a programmable nuclease that exhibits sequence independent cleavage upon forming a complex comprising the segment of the guide nucleic acid binding to the segment of the target nucleic acid; and assaying for a signal indicating cleavage of at least some protein-nucleic acids of a population of protein-nucleic acids, wherein the signal indicates a presence of the target nucleic acid in the sample and wherein absence of the signal indicates an absence of the target nucleic acid in the sample. The cleaving of the nucleic acid of a reporter using the programmable nuclease may cleave with an efficiency of 50% as measured by a change in a signal that is calorimetric, potentiometric, amperometric, optical (e.g., fluorescent, colorimetric, etc.), or piezo-electric, as non-limiting examples. Some methods as described herein can be a method of detecting a target nucleic acid in a sample comprising contacting the sample comprising the target nucleic acid with a guide nucleic acid targeting a target nucleic acid segment, a programmable nuclease capable of being activated when complexed with the guide nucleic acid and the target nucleic acid segment, a single stranded nucleic acid of a reporter comprising a detection moiety, wherein the nucleic acid of a reporter is capable of being cleaved by the activated programmable nuclease, thereby generating a first detectable signal, cleaving the single stranded nucleic acid of a reporter using the programmable nuclease that cleaves as measured by a change in color, and measuring the first detectable signal on the support medium. The cleaving of the single stranded nucleic acid of a reporter using the programmable nuclease may cleave with an efficiency of 50% as measured by a change in color. In some cases, the cleavage efficiency is at least 40%, 50%, 60%, 70%, 80%, 90%, or 95% as measured by a change in color. The change in color may be a detectable colorimetric signal or a signal visible by eye. The change in color may be measured as a first detectable signal. The first detectable signal can be detectable within 5 minutes of contacting the sample comprising the target nucleic acid with a guide nucleic acid targeting a target nucleic acid segment, a programmable nuclease capable of being activated when complexed with the guide nucleic acid and the target nucleic acid segment, and a single stranded nucleic acid of a reporter comprising a detection moiety, wherein the nucleic acid of a reporter is capable of being cleaved by the activated nuclease. The first detectable signal can be detectable within 1, 2, 3, 4, 5, 6, 7, 8, 9, 10, 11, 12, 13, 14, 15, 20, 25, 30, 35, 40, 45, 50, 55, 60, 70, 80, 90, 100, 110, or 120 minutes of contacting the sample. In some embodiments, the first detectable signal can be detectable within from 1 to 120, from 5 to 100, from 10 to 90, from 15 to 80, from 20 to 60, or from 30 to 45 minutes of contacting the sample.

In some cases, the methods, reagents, enzymes, and kits described herein detect a target single-stranded nucleic acid with a programmable nuclease and a single-stranded nucleic acid of a reporter in a sample where the sample is contacted with the reagents for a predetermined length of time sufficient for trans-cleavage of the single stranded nucleic acid of a reporter.

Some methods as described herein can be a method of detecting a target nucleic acid in a sample comprising contacting the sample comprising the target nucleic acid with a guide nucleic acid targeting a target sequence, a programmable nuclease capable of being activated when complexed with the guide nucleic acid and the target sequence, a single stranded reporter nucleic acid comprising a detection moiety, wherein the reporter nucleic acid is capable of being cleaved by the activated nuclease, thereby generating a first detectable signal, cleaving the single stranded reporter nucleic acid using the programmable nuclease that cleaves as measured by a change in color, and measuring the first detectable signal on the support medium. The cleaving of the single stranded reporter nucleic acid using the programmable nuclease may cleave with an efficiency of 50% as measured by a change in color. In some cases, the cleavage efficiency is at least 40%, at least 50%, at least 60%, at least 70%, at least 80%, at least 90%, or at least 95% as measured by a change in color. The change in color may be a detectable colorimetric signal or a signal visible by eye. The change in color may be measured as a first detectable signal. The first detectable signal can be detectable within 5 minutes of contacting the sample comprising the target nucleic acid with a guide nucleic acid targeting a target sequence, a programmable nuclease capable of being activated when complexed with the guide nucleic acid and the target sequence, and a single stranded reporter nucleic acid comprising a detection moiety, wherein the reporter nucleic acid is capable of being cleaved by the activated nuclease. The first detectable signal can be detectable within 1, 2, 3, 4, 5, 6, 7, 8, 9, 10, 11, 12, 13, 14, 15, 20, 25, 30, 35, 40, 45, 50, 55, 60, 70, 80, 90, 100, 110, or 120 minutes of contacting the sample.

### Multiplexing Programmable Nucleases and Programmable Nickases

Described herein are compositions comprising a programmable nuclease (e.g., a CasΦ as disclosed herein) capable of being activated when complexed with the guide nucleic acid and the target nucleic acid molecule. Furthermore, these reagents can be used with different types of programmable nuclease, e.g., for multiplexing programmable nucleases. In some embodiments, the programmable nucleases can exist in RNP complexes that target multiple genes simultaneously. In some embodiments, a programmable nickase may be multiplexed with an additional programmable nuclease. For example, a programmable nickase may be multiplexed with an additional programmable nuclease for modification or detection of a target nucleic acid. In some embodiments, a first programmable nickase may be multiplexed with a second programmable nickase. In some embodiments, the programmable nickase may be a CasΦ programmable nickase.

In some embodiments, a CasΦ polypeptide disclosed herein may be multiplexed with multiple guide nucleic acids in the same sample, wherein the guide nucleic acids may comprise different sequences.

In some embodiments, an additional programmable nuclease used in multiplexing is any suitable programmable nuclease. Sometimes, the programmable nuclease is any Cas protein (also referred to as a Cas nuclease herein). In some cases, the programmable nuclease is Cas13. In some embodiments, the Cas13 is Cas13a, Cas13b, Cas13c, Cas13d, or Cas13e. In some cases, the programmable nuclease can be Mad7 or Mad2. In some cases, the programmable nuclease is a Cas12 protein. Sometimes the Cas12 is Cas12a, Cas12b, Cas12c, Cas12d, Cas12e, Cas12g, Cas12h, or Cas12i. In some cases, the programmable nuclease is another CasΦ protein. In some cases, the programmable nuclease is Csm1, Cas9, C2c4, C2c8, C2c5, C2c10, C2c9, or CasZ. Sometimes, the Csm1 can be also called smCms1, miCms1, obCms1, or suCms1. Sometimes CasZ can be also called Cas14a, Cas14b, Cas14c, Cas14d, Cas14e, Cas14f, Cas14g, or Cas14h. Sometimes, the programmable nuclease can be a type V CRISPR-Cas system. In some cases, the programmable nuclease can be a type VI CRISPR-Cas system. Sometimes the programmable nuclease can be a type III CRISPR-Cas system.

In some cases, an additional programmable nuclease used in multiplexing can be from, for example, Leptotrichia shahii (Lsh), Listeria seeligeri (Lse), Leptotrichia buccalis (Lbu), Leptotrichia wadeu (Lwa), Rhodobacter capsulatus (Rca), Herbinix hemicellulosilytica (Hhe), Paludibacter propionicigenes (Ppr), Lachnospiraceae bacterium (Lba), Eubacterium rectale (Ere), Listeria newyorkensis (Lny), Clostridium aminophilum (Cam), Prevotella sp. (Psm), Capnocytophaga canimorsus (Cca, Lachnospiraceae bacterium (Lba), Bergeyella zoohelcum (Bzo), Prevotella intermedia (Pin), Prevotella buccae (Pbu), Alistipes sp. (Asp), Riemerella anatipestifer (Ran), Prevotella aurantiaca (Pau), Prevotella saccharolytica (Psa), Prevotella intermedia (Pin2), Capnocytophaga canimorsus (Cca), Porphyromonas gulae (Pgu), Prevotella sp. (Psp), Porphyromonas gingivalis (Pig), Prevotella intermedia (Pin3), Enterococcus italicus (Ei), Lactobacillus salivarius (Ls), or Thermus thermophilus (Tt). In some cases, an additional programmable nuclease used in multiplexing can be from, for example, a phage such as a bacteriophage also called a megaphage. The nucleases may come from a particular bacteriophage clade called Biggiephage. Any combination of programmable nucleases can be used in multiplexing. In some embodiments, multiplexing of programmable nucleases takes place in one reaction volume. In other embodiments, multiplexing of programmable nucleases takes place in separate reaction volumes in a single device.

### Amplification of a Target Nucleic Acid

Disclosed herein are methods of amplifying a target nucleic acid for detection using any of the methods, reagents, kits or devices described herein. The compositions for amplification of target nucleic acids and methods of use thereof, as described herein, are compatible with the DETECTR assay methods disclosed herein. The compositions for amplification of target nucleic acids and methods of use thereof, as described herein, are compatible with any of the programmable nucleases disclosed herein and use of said programmable nuclease in a method of detecting a target nucleic acid. A target nucleic acid can be an amplified nucleic acid of interest. The nucleic acid of interest may be any nucleic acid disclosed herein or from any sample as disclosed herein. This amplification can be thermal amplification (e.g., using PCR) or isothermal amplification. This nucleic acid amplification of the sample can improve at least one of sensitivity, specificity, or accuracy of the detection the target nucleic acid. The reagents for nucleic acid amplification can comprise a recombinase, an oligonucleotide primer, a single-stranded DNA binding (SSB) protein, and a polymerase. The nucleic acid amplification can be transcription mediated amplification (TMA). Nucleic acid amplification can be helicase dependent amplification (HDA) or circular helicase dependent amplification (cHDA). In additional cases, nucleic acid amplification is strand displacement amplification (SDA). The nucleic acid amplification can be recombinase polymerase amplification (RPA). The nucleic acid amplification can be at least one of loop mediated amplification (LAMP) or the exponential amplification reaction (EXPAR). Nucleic acid amplification is, in some cases, by rolling circle amplification (RCA), ligase chain reaction (LCR), simple method amplifying RNA targets (SMART), single primer isothermal amplification (SPIA), multiple displacement amplification (MDA), nucleic acid sequence based amplification (NASBA), hinge-initiated primer-dependent amplification of nucleic acids (HIP), nicking enzyme amplification reaction (NEAR), or improved multiple displacement amplification (IMDA). The nucleic acid amplification can be performed for no greater than 1, 2, 3, 4, 5, 6, 7, 8, 9, 10, 11, 12, 13, 14, 15, 16, 17, 18, 19, 20, 25, 30, 40, 50, or 60 minutes. Sometimes, the nucleic acid amplification reaction is performed at a temperature of around 20-45° C. The nucleic acid amplification reaction can be performed at a temperature no greater than 20° C., 25° C., 30° C., 35° C., 37° C., 40° C., 45° C. The nucleic acid amplification reaction can be performed at a temperature of at least 20° C., 25° C., 30° C., 35° C., 37° C., 40° C., or 45° C.

The compositions for amplification of target nucleic acids and methods of use thereof, as described herein, are compatible with any of the compositions comprising a programmable nuclease and a buffer, which has been developed to improve the function of the programmable nuclease and use of said compositions in a method of detecting a target nucleic acid. The compositions for amplification of target nucleic acids and methods of use thereof, as described herein, are compatible with any of the methods disclosed herein including methods of assaying for at least one base difference (e.g., assaying for a SNP or a base mutation) in a target nucleic acid sequence, methods of assaying for a target nucleic acid that lacks a PAM by amplifying the target nucleic acid sequence to introduce a PAM, and compositions used in introducing a PAM via amplification into the target nucleic acid sequence. In some cases, amplification of the target nucleic acid may increase the sensitivity of a detection reaction. In some cases, amplification of the target nucleic acid may increase the specificity of a detection reaction. Amplification of the target nucleic acid may increase the concentration of the target nucleic acid in the sample relative to the concentration of nucleic acids that do not correspond to the target nucleic acid. In some embodiments, amplification of the target nucleic acid may be used to modify the sequence of the target nucleic acid. For example, amplification may be used to insert a PAM sequence into a target nucleic acid that lacks a PAM sequence. In some cases, amplification may be used to increase the homogeneity of a target nucleic acid sequence. For example, amplification may be used to remove a nucleic acid variation that is not of interest in the target nucleic acid sequence.

An amplified target nucleic acid may be present in a DETECTR reaction in an amount relative to an amount of a programmable nuclease. In some embodiments, the amplified target nucleic acid is present in at least 1-fold, 2-fold, 3-fold, 4-fold, 5-fold, 10-fold, 25-fold, 50-fold, 100-fold, 500-fold, 1000-fold, 10,000-fold, or 100,000-fold molar excess relative to the amount of the programmable nuclease. In some embodiments, the amplified target nucleic acid is present in no more than 1-fold, 2-fold, 3-fold, 4-fold, 5-fold, 10-fold, 25-fold, 50-fold, 100-fold, 500-fold, 1000-fold, 10,000-fold, or 100,000-fold molar excess relative to the amount of the programmable nuclease. In some embodiments, the amplified target nucleic acid is present in from 1-fold to 2-fold, from 1-fold to 3-fold, from 1-fold to 4-fold, from 1-fold to 5-fold, from 1-fold to 10-fold, from 1-fold to 25-fold, from 1-fold to 50-fold, from 1-fold to 100-fold, from 1-fold to 500-fold, from 1-fold to 1000-fold, from 1-fold to 10,000-fold, from 1-fold to 100,000-fold, from 5-fold to 10-fold, from 5-fold to 25-fold, from 5-fold to 50-fold, from 5-fold to 100-fold, from 5-fold to 500-fold, from 5-fold to 1000-fold, from 5-fold to 10,000-fold, from 5-fold to 100,000-fold, from 10-fold to 25-fold, from 10-fold to 50-fold, from 10-fold to 100-fold, from 10-fold to 500-fold, from 10-fold to 1000-fold, from 10-fold to 10,000-fold, from 10-fold to 100,000-fold, from 100-fold to 500-fold, from 100-fold to 1000-fold, from 100-fold to 10,000-fold, from 100-fold to 100,000-fold, from 1000-fold to 10,000-fold, from 1000-fold to 100,000-fold, or from 10,000-fold to 100,000-fold molar excess relative to the amount of the programmable nuclease. In some embodiments, the programmable nuclease is present in at least 1-fold, 2-fold, 3-fold, 4-fold, 5-fold, 10-fold, 25-fold, 50-fold, 100-fold, 500-fold, 1000-fold, 10,000-fold, or 100,000-fold molar excess relative to the amount of the target nucleic acid. In some embodiments, the programmable nuclease is present in no more than 1-fold, 2-fold, 3-fold, 4-fold, 5-fold, 10-fold, 25-fold, 50-fold, 100-fold, 500-fold, 1000-fold, 10,000-fold, or 100,000-fold molar excess relative to the amount of the target nucleic acid. In some embodiments, the programmable nuclease is present in from 1-fold to 2-fold, from 1-fold to 3-fold, from 1-fold to 4-fold, from 1-fold to 5-fold, from 1-fold to 10-fold, from 1-fold to 25-fold, from 1-fold to 50-fold, from 1-fold to 100-fold, from 1-fold to 500-fold, from 1-fold to 1000-fold, from 1-fold to 10,000-fold, from 1-fold to 100,000-fold, from 5-fold to 10-fold, from 5-fold to 25-fold, from 5-fold to 50-fold, from 5-fold to 100-fold, from 5-fold to 500-fold, from 5-fold to 1000-fold, from 5-fold to 10,000-fold, from 5-fold to 100,000-fold, from 10-fold to 25-fold, from 10-fold to 50-fold, from 10-fold to 100-fold, from 10-fold to 500-fold, from 10-fold to 1000-fold, from 10-fold to 10,000-fold, from 10-fold to 100,000-fold, from 100-fold to 500-fold, from 100-fold to 1000-fold, from 100-fold to 10,000-fold, from 100-fold to 100,000-fold, from 1000-fold to 10,000-fold, from 1000-fold to 100,000-fold, or from 10,000-fold to 100,000-fold molar excess relative to the amount of the target nucleic acid. In some embodiments, the target nucleic acid is not present in the sample.

An amplified target nucleic acid may be present in a DETECTR reaction in an amount relative to an amount of a guide nucleic acid. In some embodiments, the amplified target nucleic acid is present in at least 1-fold, 2-fold, 3-fold, 4-fold, 5-fold, 10-fold, 25-fold, 50-fold, 100-fold, 500-fold, 1000-fold, 10,000-fold, or 100,000-fold molar excess relative to the amount of the guide nucleic acid. In some embodiments, the amplified target nucleic acid is present in no more than 1-fold, 2-fold, 3-fold, 4-fold, 5-fold, 10-fold, 25-fold, 50-fold, 100-fold, 500-fold, 1000-fold, 10,000-fold, or 100,000-fold molar excess relative to the amount of the guide nucleic acid. In some embodiments, the amplified target nucleic acid is present in from 1-fold to 2-fold, from 1-fold to 3-fold, from 1-fold to 4-fold, from 1-fold to 5-fold, from 1-fold to 10-fold, from 1-fold to 25-fold, from 1-fold to 50-fold, from 1-fold to 100-fold, from 1-fold to 500-fold, from 1-fold to 1000-fold, from 1-fold to 10,000-fold, from 1-fold to 100,000-fold, from 5-fold to 10-fold, from 5-fold to 25-fold, from 5-fold to 50-fold, from 5-fold to 100-fold, from 5-fold to 500-fold, from 5-fold to 1000-fold, from 5-fold to 10,000-fold, from 5-fold to 100,000-fold, from 10-fold to 25-fold, from 10-fold to 50-fold, from 10-fold to 100-fold, from 10-fold to 500-fold, from 10-fold to 1000-fold, from 10-fold to 10,000-fold, from 10-fold to 100,000-fold, from 100-fold to 500-fold, from 100-fold to 1000-fold, from 100-fold to 10,000-fold, from 100-fold to 100,000-fold, from 1000-fold to 10,000-fold, from 1000-fold to 100,000-fold, or from 10,000-fold to 100,000-fold molar excess relative to the amount of the guide nucleic acid. In some embodiments, the guide nucleic acid is present in at least 1-fold, 2-fold, 3-fold, 4-fold, 5-fold, 10-fold, 25-fold, 50-fold, 100-fold, 500-fold, 1000-fold, 10,000-fold, or 100,000-fold molar excess relative to the amount of the target nucleic acid. In some embodiments, the guide nucleic acid is present in no more than 1-fold, 2-fold, 3-fold, 4-fold, 5-fold, 10-fold, 25-fold, 50-fold, 100-fold, 500-fold, 1000-fold, 10,000-fold, or 100,000-fold molar excess relative to the amount of the target nucleic acid. In some embodiments, the guide nucleic acid is present in from 1-fold to 2-fold, from 1-fold to 3-fold, from 1-fold to 4-fold, from 1-fold to 5-fold, from 1-fold to 10-fold, from 1-fold to 25-fold, from 1-fold to 50-fold, from 1-fold to 100-fold, from 1-fold to 500-fold, from 1-fold to 1000-fold, from 1-fold to 10,000-fold, from 1-fold to 100,000-fold, from 5-fold to 10-fold, from 5-fold to 25-fold, from 5-fold to 50-fold, from 5-fold to 100-fold, from 5-fold to 500-fold, from 5-fold to 1000-fold, from 5-fold to 10,000-fold, from 5-fold to 100,000-fold, from 10-fold to 25-fold, from 10-fold to 50-fold, from 10-fold to 100-fold, from 10-fold to 500-fold, from 10-fold to 1000-fold, from 10-fold to 10,000-fold, from 10-fold to 100,000-fold, from 100-fold to 500-fold, from 100-fold to 1000-fold, from 100-fold to 10,000-fold, from 100-fold to 100,000-fold, from 1000-fold to 10,000-fold, from 1000-fold to 100,000-fold, or from 10,000-fold to 100,000-fold molar excess relative to the amount of the target nucleic acid. In some embodiments, the target nucleic acid is not present in the sample.

### Kits

Disclosed herein are kits for use to detect, modify, edit, or regulate a target nucleic acid sequence as disclosed herein using the methods as discuss above. In some embodiments, the kit comprises the programmable nuclease system, reagents, and the support medium. The reagents and programmable nuclease system can be provided in a reagent chamber or on the support medium. Alternatively, the reagent and programmable nuclease system can be placed into the reagent chamber or the support medium by the individual using the kit. Optionally, the kit further comprises a buffer and a dropper. The reagent chamber can be a test well or container. The opening of the reagent chamber can be large enough to accommodate the support medium. The buffer can be provided in a dropper bottle for ease of dispensing. The dropper can be disposable and transfer a fixed volume. The dropper can be used to place a sample into the reagent chamber or on the support medium.

The kit or system for detection of a target nucleic acid described herein further comprises reagents for nucleic acid amplification of target nucleic acids in the sample. Isothermal nucleic acid amplification allows the use of the kit or system in remote regions or low resource settings without specialized equipment for amplification. Often, the reagents for nucleic acid amplification comprise a recombinase, an oligonucleotide primer, a single-stranded DNA binding (SSB) protein, and a polymerase. Sometimes, nucleic acid amplification of the sample improves at least one of sensitivity, specificity, or accuracy of the assay in detecting the target nucleic acid. In some cases, the nucleic acid amplification is performed in a nucleic acid amplification region on the support medium. Alternatively, or in combination, the nucleic acid amplification is performed in a reagent chamber, and the resulting sample is applied to the support medium. Sometimes, the nucleic acid amplification is isothermal nucleic acid amplification. In some cases, the nucleic acid amplification is transcription mediated amplification (TMA). Nucleic acid amplification is helicase dependent amplification (HDA) or circular helicase dependent amplification (cHDA) in other cases. In additional cases, nucleic acid amplification is strand displacement amplification (SDA). In some cases, nucleic acid amplification is by recombinase polymerase amplification (RPA). In some cases, nucleic acid amplification is by at least one of loop mediated amplification (LAMP) or the exponential amplification reaction (EXPAR). Nucleic acid amplification is, in some cases, by rolling circle amplification (RCA), ligase chain reaction (LCR), simple method amplifying RNA targets (SMART), single primer isothermal amplification (SPIA), multiple displacement amplification (MDA), nucleic acid sequence based amplification (NASBA), hinge-initiated primer-dependent amplification of nucleic acids (HIP), nicking enzyme amplification reaction (NEAR), or improved multiple displacement amplification (IMDA). Often, the nucleic acid amplification is performed for no greater than 1, 2, 3, 4, 5, 6, 7, 8, 9, 10, 11, 12, 13, 14, 15, 16, 17, 18, 19, 20, 25, 30, 40, 50, or 60 minutes, or any value from 1 to 60 minutes. Sometimes, the nucleic acid amplification is performed for from 1 to 60, from 5 to 55, from 10 to 50, from 15 to 45, from 20 to 40, or from 25 to 35 minutes. Sometimes, the nucleic acid amplification reaction is performed at a temperature of around 20-45° C. In some cases, the nucleic acid amplification reaction is performed at a temperature no greater than 20° C., 25° C., 30° C., 35° C., 37° C., 40° C., 45° C., or any value from 20° C. to 45° C. In some cases, the nucleic acid amplification reaction is performed at a temperature of at least 20° C., 25° C., 30° C., 35° C., 37° C., 40° C., or 45° C., or any value from 20° C. to 45° C. In some cases, the nucleic acid amplification reaction is performed at a temperature of from 20° C. to 45° C., from 25° C. to 40° C., from 30° C. to 40° C., or from 35° C. to 40° C.

In some embodiments, a kit for detecting a target nucleic acid comprising a support medium; a guide nucleic acid targeting a target sequence; a programmable nuclease capable of being activated when complexed with the guide nucleic acid and the target sequence; and a reporter nucleic acid comprising a detection moiety, wherein the reporter nucleic acid is capable of being cleaved by the activated nuclease, thereby generating a first detectable signal. Often, the kit further comprises primers for amplifying a target nucleic acid of interest to produce a PAM target nucleic acid.

In some embodiments, a kit for detecting a target nucleic acid comprising a PCR plate; a guide nucleic acid targeting a target sequence; a programmable nuclease capable of being activated when complexed with the guide nucleic acid and the target sequence; and a single stranded reporter nucleic acid comprising a detection moiety, wherein the reporter nucleic acid is capable of being cleaved by the activated nuclease, thereby generating a first detectable signal. The wells of the PCR plate can be pre-aliquoted with the guide nucleic acid targeting a target sequence, a programmable nuclease capable of being activated when complexed with the guide nucleic acid and the target sequence, and at least one population of a single stranded reporter nucleic acid comprising a detection moiety. A user can thus add the biological sample of interest to a well of the pre-aliquoted PCR plate and measure for the detectable signal with a fluorescent light reader or a visible light reader.

In some embodiments, a kit for modifying a target nucleic acid comprising a support medium; a guide nucleic acid targeting a target sequence; and a programmable nuclease capable of being activated when complexed with the guide nucleic acid and the target sequence.

In some embodiments, a kit for modifying a target nucleic acid comprising a PCR plate; a guide nucleic acid targeting a target sequence; and a programmable nuclease capable of being activated when complexed with the guide nucleic acid and the target sequence. The wells of the PCR plate can be pre-aliquoted with the guide nucleic acid targeting a target sequence, and a programmable nuclease capable of being activated when complexed with the guide nucleic acid and the target sequence. A user can thus add the biological sample of interest to a well of the pre-aliquoted PCR plate.

In some instances, such kits may include a package, carrier, or container that is compartmentalized to receive one or more containers such as vials, tubes, and the like, each of the container(s) comprising one of the separate elements to be used in a method described herein.

Suitable containers include, for example, test wells, bottles, vials, and test tubes. In one embodiment, the containers are formed from a variety of materials such as glass, plastic, or polymers.

The kit or systems described herein contain packaging materials. Examples of packaging materials include, but are not limited to, pouches, blister packs, bottles, tubes, bags, containers, bottles, and any packaging material suitable for intended mode of use.

A kit typically includes labels listing contents and/or instructions for use, and package inserts with instructions for use. A set of instructions will also typically be included. In one embodiment, a label is on or associated with the container. In some instances, a label is on a container when letters, numbers or other characters forming the label are attached, molded or etched into the container itself, a label is associated with a container when it is present within a receptacle or carrier that also holds the container, e.g., as a package insert. In one embodiment, a label is used to indicate that the contents are to be used for a specific therapeutic application. The label also indicates directions for use of the contents, such as in the methods described herein.

After packaging the formed product and wrapping or boxing to maintain a sterile barrier, the product may be terminally sterilized by heat sterilization, gas sterilization, gamma irradiation, or by electron beam sterilization. Alternatively, the product may be prepared and packaged by aseptic processing.

Unless otherwise defined, all technical terms used herein have the same meaning as commonly understood by one of ordinary skill in the art to which this invention belongs. As used in this specification and the appended claims, the singular forms “a,” “an,” and “the” include plural references unless the context clearly dictates otherwise. Any reference to “or” herein is intended to encompass “and/or” unless otherwise stated.

As used herein, the term “comprising” and its grammatical equivalents specifies the presence of stated features, integers, steps, operations, elements, and/or components, but do not preclude the presence or addition of one or more other features, integers, steps, operations, elements, components, and/or groups thereof. As used herein, the term “and/or” includes any and all combinations of one or more of the associated listed items.

Unless specifically stated or obvious from context, as used herein, the term “about” in reference to a number or range of numbers is understood to mean the stated number and numbers +/−10% thereof, or 10% below the lower listed limit and 10% above the higher listed limit for the values listed for a range.

As used herein the terms “individual,” “subject,” and “patient” are used interchangeably and include any member of the animal kingdom, including humans.

Methods of the disclosure can be performed in a subject. Compositions of the disclosure can be administered to a subject. A subject can be a human. A subject can be a mammal (e.g., rat, mouse, cow, dog, pig, sheep, horse). A subject can be a vertebrate or an invertebrate. A subject can be a laboratory animal. A subject can be a patient. A subject can be suffering from a disease. A subject can display symptoms of a disease. A subject may not display symptoms of a disease, but still have a disease. A subject can be under medical care of a caregiver (e.g., the subject is hospitalized and is treated by a physician). A subject can be a plant or a crop.

Methods of the disclosure can be performed in a cell. A cell can be in vitro. A cell can be in vivo. A cell can be ex vivo. A cell can be an isolated cell. A cell can be a cell inside of an organism. A cell can be an organism. A cell can be a cell in a cell culture. A cell can be one of a collection of cells. A cell can be a mammalian cell or derived from a mammalian cell. A cell can be a rodent cell or derived from a rodent cell. A cell can be a human cell or derived from a human cell. A cell can be a prokaryotic cell or derived from a prokaryotic cell. A cell can be a bacterial cell or can be derived from a bacterial cell. A cell can be an archaeal cell or derived from an archaeal cell. A cell can be a eukaryotic cell or derived from a eukaryotic cell. A cell can be a pluripotent stem cell. A cell can be a plant cell or derived from a plant cell. A cell can be an animal cell or derived from an animal cell. A cell can be an invertebrate cell or derived from an invertebrate cell. A cell can be a vertebrate cell or derived from a vertebrate cell. A cell can be a microbe cell or derived from a microbe cell. A cell can be a fungi cell or derived from a fungi cell. A cell can be from a specific organ or tissue.

Methods of the disclosure can be performed in a eukaryotic cell or cell line. In some embodiments, the eukaryotic cell is a Chinese hamster ovary (CHO) cell. In some embodiments, the eukaryotic cell is a Human embryonic kidney 293 cells (also referred to as HEK or HEK 293) cell. In some embodiments, the eukaryotic cell is a K562 cell.

Non-limiting examples of cell lines that can be used with the disclosure include C8161, CCRF-CEM, MOLT, mIMCD-3, NHDF, HeLa-S3, Huh1, Huh4, Huh7, HUVEC, HASMC, HEKn, HEKa, MiaPaCell, Panc1, PC-3, TF1, CTLL-2, CIR, Rat6, CV1, RPTE, A10, T24, J82, A375, ARH-77, Calu1, SW480, SW620, SKOV3, SK-UT, CaCo2, P388D1, SEM-K2, WEHI-231, HB56, TIB55, Jurkat, J45.01, LRMB, Bcl-1, BC-3, IC21, DLD2, Raw264.7, NRK, NRK-52E, MRC5, MEF, Hep G2, HeLa B, HeLa T4, COS, COS-1, COS-6, COS-M6A, BS-C-1 monkey kidney epithelial, BALB/3T3 mouse embryo fibroblast, 3T3 Swiss, 3T3-L1, 132-d5 human fetal fibroblasts; 10.1 mouse fibroblasts, 293-T, 3T3, 721, 9L, A2780, A2780ADR, A2780cis, A172, A20, A253, A431, A-549, ALC, B16, B35, BCP-1 cells, BEAS-2B, bEnd.3, BHK-21, BR 293, BxPC3, C3H-10T1/2, C6/36, Cal-27, CHO, CHO-7, CHO-IR, CHO-K1, CHO-K2, CHO-T, CHO Dhfr −/−, COR-L23, COR-L23/CPR, COR-L23/5010, COR-L23/R23, COS-7, COV-434, CML T1, CMT, CT26, D17, DH82, DU145, DuCaP, EL4, EM2, EM3, EMT6/AR1, EMT6/AR10.0, FM3, H1299, H69, HB54, HB55, HCA2, HEK-293, HeLa, Hepa1-6, Hepa1c1c7, HL-60, HMEC, HT-29, Jurkat, JY cells, K562 cells, Ku812, KCL22, KG1, KYO1, LNCap, Ma-Mel 1-48, MC-38, MCF-7, MCF-10A, MDA-MB-231, MDA-MB-468, MDA-MB-435, MDCK II, MDCK II, MOR/0.2R, MONO-MAC 6, MTD-1A, MyEnd, NCI-H69/CPR, NCI-H69/LX10, NCI-H69/LX20, NCI-H69/LX4, NIH-3T3, NALM-1, NW-145, OPCN/OPCT cell lines, Peer, PNT-1A/PNT 2, RenCa, RIN-5F, RMA/RMAS, Saos-2 cells, Sf-9, SkBr3, T2, T-47D, T84, THP1 cell line, U373, U87, U937, VCaP, Vero cells, WM39, WT-49, X63, YAC-1, and YAR. Non-limiting examples of other cells that can be used with the disclosure include immune cells, such as CART, T-cells, B-cells, NK cells, granulocytes, basophils, eosinophils, neutrophils, mast cells, monocytes, macrophages, dendritic cells, antigen-presenting cells (APC), or adaptive cells. Non-limiting examples of cells that can be used with this disclosure also include plant cells, such as Parenchyma, sclerenchyma, collenchyma, xylem, phloem, germline (e.g., pollen). Cells from lycophytes, ferns, gymnosperms, angiosperms, bryophytes, charophytes, chloropytes, rhodophytes, or glaucophytes. Non-limiting examples of cells that can be used with this disclosure also include stem cells, such as human stem cells, animal stem cells, stem cells that are not derived from human embryonic stem cells, embryonic stem cells, mesenchymal stem cells, pluripotent stem cells, induced pluripotent stem cells (iPS), somatic stem cells, adult stem cells, hematopoietic stem cells, tissue-specific stem cells.

Methods described herein may be used to create populations of cells comprising at least one of the cells described herein. In some cases, a population of cells comprises a non-naturally occurring compositions described herein.

Compositions of the disclosure include populations of cells, or any progeny thereof, comprising other compositions described herein or that have been modified by the methods described herein.

Methods described herein may include producing a protein from a cell or a population of cells described herein. In some cases, the method comprises producing a protein, and industrial protein, or a protein at large scale using a cell provided for herein that has been modified by any of the methods described herein. In some cases, a rodent cell or CHO cell is modified by a nuclease or cas enzyme described herein and is later used, expanded, or cultured for protein production. In some cases, a derivative or progeny of a modified CHO cell, as described herein, is used, expanded, or cultured for protein production. A method of protein production may further comprise a donor template, additional guide RNA, a buffer, a protease inhibitor, a nuclease inhibitor, or a detergent.

### EXAMPLES

The following examples are included to further describe some aspects of the present disclosure and should not be used to limit the scope of the invention.

### Example 1

### Human Codon Optimized CasΦ Polypeptide

Human codon-optimized nucleotide sequences of illustrative CasΦ polypeptides were prepared. TABLE 4 provides human codon optimized nucleotide sequences of illustrative CasΦ polypeptides that are suitable for use with the methods and compositions of the disclosure.

TABLE 4
Human codon optimized nucleotide sequences

| | Endogenous Amino | |
|---|---|---|
| Name | Acid Sequence | Human Codon Optimized Nucleotide Sequence |
| CasΦ.2 | MPKPAVESEFSKVLK | ATGCCTAAGCCTGCCGTGGAAAGCGAGTTCAG |
| | KHFPGERFRSSYMKR | CAAGGTGCTGAAGAAGCACTTCCCCGGCGAGC |
| | GGKILAAQGEEAVVA | GGTTCAGATCCAGCTACATGAAGAGAGGCGGC |
| | YLQGKSEEEPPNFQPP | AAGATCCTGGCCGCTCAAGGCGAAGAAGCCGT |
| | AKCHVVTKSRDFAE | GGTCGCATATCTGCAGGGCAAGAGCGAGGAA |
| | WPIMKASEAIQRYIYA | GAACCTCCTAACTTCCAGCCTCCTGCCAAGTG |
| | LSTTERAACKPGKSSE | CCACGTGGTCACCAAGAGCAGAGATTTCGCCG |
| | SHAAWFAATGVSNH | AGTGGCCCATCATGAAGGCCTCTGAAGCCATC |
| | GYSHVQGLNLIFDHT | CAGCGGTACATCTACGCCCTGAGCACAACAGA |
| | LGRYDGVLKKVQLR | AAGAGCCGCCTGCAAGCCTGGCAAGAGCAGC |
| | NEKARARLESINASR | GAATCTCACGCCGCTTGGTTTGCCGCTACCGG |
| | ADEGLPEIKAEEEEVA | CGTGTCCAATCACGGCTACTCTCATGTGCAGG |
| | TNETGHLLQPPGINPS | GCCTGAACCTGATCTTCGATCACACCCTGGGC |
| | FYVYQTISPQAYRPRD | AGATACGACGGCGTGCTGAAAAAGGTGCAGC |
| | EIVLPPEYAGYVRDPN | TGCGGAACGAGAAGGCCAGAGCCAGACTGGA |
| | APIPLGVVRNRCDIQK | ATCCATCAACGCCAGCAGAGCCGATGAGGGCC |
| | GCPGYIPEWQREAGT | TGCCTGAGATTAAGGCCGAAGAGGAAGAGGT |
| | AISPKTGKAVTVPGLS | GGCCACAAACGAAACCGGCCATCTGCTGCAGC |
| | PKKNKRMRRYWRSE | CACCTGGCATCAACCCTAGCTTCTACGTGTAC |
| | KEKAQDALLVTVRIG | CAGACAATCAGCCCTCAGGCCTACAGACCCAG |
| | TDWVVIDVRGLLRNA | GGACGAGATTGTGCTGCCTCCTGAGTATGCCG |
| | RWRTIAPKDISLNALL | GCTACGTGCGGGATCCCAACGCTCCTATTCCT |
| | DLFTGDPVIDVRRNIV | CTGGGCGTCGTGCGGAACAGATGCGACATCCA |
| | TFTYTLDACGTYARK | GAAAGGCTGCCCCGGCTACATTCCCGAGTGGC |
| | WTLKGKQTKATLDK | AGAGAGAAGCCGGCACCGCCATTTCTCCAAAG |
| | LTATQTVALVAIDLG | ACAGGCAAAGCCGTGACCGTGCCTGGCCTGTC |
| | QTNPISAGISRVTQEN | TCCTAAGAAAAACAAGCGGATGCGGCGGTACT |
| | GALQCEPLDRFTLPD | GGCGGAGCGAGAAAGAAAAAGCCCAGGACGC |
| | DLLKDISAYRIAWDR | CCTGCTGGTCACAGTGCGGATTGGCACAGATT |
| | NEEELRARSVEALPE | GGGTCGTGATCGATGTGCGCGGCCTGCTGAGA |
| | AQQAEVRALDGVSKE | AATGCCAGATGGCGGACAATCGCCCCTAAGGA |
| | TARTQLCADFGLDPK | CATCAGCCTGAACGCACTGCTGGACCTGTTCA |
| | RLPWDKMSSNTTFISE | CCGGCGATCCTGTGATTGACGTGCGGCGGAAC |
| | ALLSNSVSRDQVFFTP | ATCGTGACCTTCACCTACACACTGGACGCCTG |
| | APKKGAKKKAPVEV | CGGCACCTACGCCAGAAAGTGGACACTGAAG |
| | MRKDRTWARAYKPR | GGCAAGCAGACCAAGGCCACTCTGGACAAGC |
| | LSVEAQKLKNEALW | TGACCGCCACACAGACAGTGGCCCTGGTGGCT |
| | ALKRTSPEYLKLSRR | ATTGATCTGGGCCAGACAAACCCTATCAGCGC |
| | KEELCRRSINYVIEKT | CGGCATCAGCAGAGTGACCCAAGAAAATGGC |
| | RRRTQCQIVIPVIEDL | GCCCTGCAGTGCGAGCCCCTGGACAGATTCAC |
| | NVRFFHGSGKRLPGW | ACTGCCCGACGACCTGCTGAAGGACATCTCCG |
| | DNFFTAKKENRWFIQ | CCTATAGAATCGCCTGGGACCGCAATGAAGAG |
| | GLHKAFSDLRTHRSF | GAACTGAGAGCCAGAAGCGTGGAAGCCCTGC |
| | YVFEVRPERTSITCPK | CTGAAGCACAGCAGGCTGAAGTGCGAGCACT |
| | CGHCEVGNRDGEAFQ | GGACGGGGTGTCCAAAGAGACAGCCAGAACT |
| | CLSCGKTCNADLDVA | CAGCTGTGCGCCGACTTTGGACTGGACCCCAA |
| | THNLTQVALTGKTMP | AAGACTGCCCTGGGACAAGATGAGCAGCAAC |
| | KREEPRDAQGTAPAR | ACCACCTTCATCAGCGAGGCCCTGCTGAGCAA |
| | KTKKASKSKAPPAER | TAGCGTGTCCAGAGATCAGGTGTTCTTCACCC |
| | EDQTPAQEPSQTS | CTGCTCCAAAGAAGGGCGCCAAGAAGAAAGC |
| | (SEQ ID NO: 2) | CCCTGTCGAAGTGATGCGGAAGGACCGGACAT |
| | | GGGCCAGAGCTTACAAGCCCAGACTGTCCGTG |
| | | GAAGCTCAGAAGCTGAAGAACGAAGCCCTGT |
| | | GGGCCCTGAAGAGAACAAGCCCCGAGTACCT |
| | | GAAGCTGAGCCGGCGGAAAGAAGAACTCTGC |
| | | CGGCGGAGCATCAACTACGTGATCGAGAAAA |
| | | CCCGGCGGAGAACCCAGTGCCAGATCGTGATT |
| | | CCTGTGATCGAGGACCTGAACGTGCGGTTCTT |
| | | TCACGGCAGCGGCAAGAGACTGCCCGGCTGG |
| | | GATAATTTCTTCACCGCCAAAAAAGAAAACCG |
| | | GTGGTTCATCCAGGGCCTGCACAAGGCCTTCA |
| | | GCGACCTGAGAACCCACCGGTCCTTTTACGTG |
| | | TTCGAAGTGCGGCCCGAGCGGACCAGCATCAC |
| | | CTGTCCTAAATGCGGCCACTGCGAAGTGGGCA |
| | | ACAGAGATGGCGAGGCCTTCCAGTGTCTGAGC |
| | | TGTGGCAAGACCTGCAACGCCGACCTGGATGT |
| | | GGCCACTCACAATCTGACACAGGTGGCCCTGA |
| | | CCGGCAAGACCATGCCTAAGAGAGAGGAACC |
| | | TAGGGACGCCCAGGGTACAGCCCCTGCCAGAA |
| | | AGACAAAGAAAGCCAGCAAGAGCAAGGCCCC |
| | | TCCTGCCGAGAGAGAAGATCAGACCCCAGCTC |
| | | AAGAGCCCAGCCAGACATCT (SEQ ID NO: 1405) |
| CasΦ.4 | MEKEITELTKIRREFP | ATGGAAAAAGAGATCACCGAGCTGACCAAGA |
| | NKKFSSTDMKKAGKL | TCCGCAGAGAGTTCCCCAACAAGAAGTTCAGC |
| | LKAEGPDAVRDFLNS | AGCACCGACATGAAGAAGGCCGGCAAGCTGC |
| | CQEIIGDFKPPVKTNI | TGAAGGCCGAAGGACCTGATGCCGTGCGGGA |
| | VSISRPFEEWPVSMVG | CTTCCTGAACAGCTGCCAAGAGATCATCGGCG |
| | RAIQEYYFSLTKEELE | ACTTCAAGCCTCCAGTCAAGACCAACATCGTG |
| | SVHPGTSSEDHKSFFN | TCCATCAGCAGACCCTTCGAGGAATGGCCCGT |
| | ITGLSNYNYTSVQGL | GTCCATGGTTGGACGGGCCATCCAAGAGTACT |
| | NLIFKNAKAIYDGTLV | ACTTCAGCCTGACCAAAGAGGAACTGGAAAG |
| | KANNKNKKLEKKEN | CGTTCACCCCGGCACCAGCAGCGAGGACCACA |
| | EINHKRSLEGLPIITPD | AGAGCTTTTTCAACATCACCGGCCTGAGCAAC |
| | FEEPFDENGHLNNPPG | TACAACTACACCAGCGTGCAGGGCCTGAACCT |
| | INRNIYGYQGCAAKV | GATCTTCAAGAACGCCAAGGCCATCTACGACG |
| | FVPSKHKMVSLPKEY | GCACCCTGGTCAAGGCCAACAACAAGAACAA |
| | EGYNRDPNLSLAGFR | GAAGCTCGAGAAGAAGTTTAACGAGATCAAC |
| | NRLEIPEGEPGHVPWF | CACAAGCGGAGCCTGGAAGGCCTGCCTATCAT |
| | QRMDIPEGQIGHVNKI | CACCCCTGATTTCGAGGAACCCTTCGACGAGA |
| | QRFNFVHGKNSGKVK | ACGGCCACCTGAACAACCCTCCAGGCATCAAC |
| | FSDKTGRVKRYHHSK | CGGAACATCTACGGCTATCAGGGCTGCGCCGC |
| | YKDATKPYKFLEESK | CAAGGTGTTCGTGCCTTCTAAGCACAAGATGG |
| | KVSALDSILAHITIGDD | TGTCCCTGCCTAAAGAGTACGAGGGCTACAAC |
| | WVVFDIRGLYRNVFY | AGGGACCCCAACCTGTCTCTGGCCGGCTTCAG |
| | RELAQKGLTAVQLLD | AAACAGACTGGAAATCCCTGAGGGCGAGCCT |
| | LFTGDPVIDPKKGVV | GGCCATGTGCCATGGTTCCAGAGAATGGATAT |
| | TFSYKEGVVPVFSQKI | CCCCGAGGGCCAGATCGGACACGTGAACAAG |
| | VPRFKSRDTLEKLTSQ | ATCCAGCGGTTCAACTTCGTGCACGGCAAGAA |
| | GPVALLSVDLGQNEP | CAGCGGCAAAGTGAAGTTCTCCGACAAGACCG |
| | VAARVCSLKNINDKIT | GCAGAGTGAAGAGATACCACCACAGCAAGTA |
| | LDNSCRISFLDDYKK | CAAGGACGCTACCAAGCCTTACAAGTTCCTGG |
| | QIKDYRDSLDELEIKI | AAGAGTCCAAGAAGGTGTCAGCCCTGGACAG |
| | RLEAINSLETNQQVEI | CATCCTGGCCATCATCACAATCGGCGACGACT |
| | RDLDVFSADRAKANT | GGGTCGTGTTCGACATCAGAGGCCTGTACCGG |
| | VDMFDIDPNLISWDS | AACGTGTTCTACAGAGAGCTGGCCCAGAAAGG |
| | MSDARVSTQISDLYL | CCTGACAGCTGTGCAACTGCTGGACCTGTTTA |
| | KNGGDESRVYFEINN | CCGGCGATCCCGTGATCGACCCCAAGAAAGGC |
| | KRIKRSDYNISQLVRP | GTGGTCACCTTCAGCTACAAAGAGGGCGTCGT |
| | KLSDSTRKNLNDSIW | CCCCGTCTTTAGCCAGAAAATCGTGCCCCGGT |
| | KLKRTSEEYLKLSKR | TCAAGAGCCGGGACACCCTGGAAAAGCTGAC |
| | KLELSRAVVNYTIRQS | CTCTCAGGGACCTGTGGCTCTGCTGTCTGTGG |
| | KLLSGINDIVIILEDLD | ACCTGGGACAGAATGAACCTGTGGCCGCCAGA |
| | VKKKFNGRGIRDIGW | GTGTGCAGCCTGAAGAACATCAACGACAAGAT |
| | DNFFSSRKENRWFIPA | CACCCTGGACAACTCTTGCCGGATCAGCTTCC |
| | FHKAFSELSSNRGLCV | TGGACGACTACAAGAAGCAGATCAAGGACTA |
| | LEVNPAWTSATCPDC | CAGAGACAGCCTGGACGAGCTGGAAATCAAG |
| | GFCSKENRDGINFTCR | ATCCGGCTGGAAGCCATCAACTCCCTCGAGAC |
| | KCGVSYHADIDVATL | AAACCAGCAGGTCGAGATCAGAGATCTGGAC |
| | NIARVAVLGKPMSGP | GTGTTCAGCGCCGACCGGGCCAAAGCCAATAC |
| | ADRERLGDTKKPRVA | CGTGGACATGTTTGACATCGACCCTAACCTGA |
| | RSRKTMKRKDISNST | TCAGCTGGGACTCCATGAGCGACGCCAGAGTC |
| | VEAMVTA (SEQ ID | AGCACCCAGATCAGCGACCTGTACCTGAAGAA |
| | NO: 4) | TGGCGGCGACGAGAGCCGGGTGTACTTTGAGA |
| | | TTAACAACAAACGGATTAAGCGGAGCGACTAC |
| | | AACATCAGCCAGCTCGTGCGGCCCAAGCTGAG |
| | | CGATAGCACCAGAAAGAACCTGAACGACAGC |
| | | ATCTGGAAGCTGAAGCGGACCAGCGAGGAAT |
| | | ACCTGAAGCTGAGCAAGCGGAAGCTGGAACT |
| | | GAGCAGAGCCGTCGTGAATTACACCATCCGGC |
| | | AGAGCAAACTGCTGAGCGGCATCAATGACATC |
| | | GTGATCATTCTCGAGGACCTGGACGTGAAGAA |
| | | GAAATTCAACGGCAGAGGCATCCGCGATATCG |
| | | GCTGGGACAACTTCTTCAGCTCCCGGAAAGAA |
| | | AACCGGTGGTTCATCCCCGCCTTCCACAAGGC |
| | | CTTTAGCGAGCTGAGCAGCAACAGGGGCCTGT |
| | | GCGTGATCGAAGTGAATCCTGCCTGGACCAGC |
| | | GCCACCTGTCCTGATTGTGGCTTCTGCAGCAA |
| | | AGAAAACAGAGATGGCATCAACTTCACGTGCC |
| | | GGAAGTGCGGCGTGTCCTACCACGCCGATATT |
| | | GACGTGGCCACACTGAATATTGCCAGAGTGGC |
| | | CGTGCTGGGCAAGCCTATGTCTGGACCTGCCG |
| | | ACAGAGAGAGACTGGGCGACACCAAGAAACC |
| | | TAGAGTGGCCCGCAGCAGAAAGACCATGAAG |
| | | CGGAAGGACATCAGCAACAGCACCGTCGAGG |
| | | CCATGGTTACAGCT (SEQ ID NO: 1406) |
| CasΦ.11 | MSNTAVSTREHMSNK | ATGAGCAACACCGCCGTGTCCACCAGAGAACA |
| | TTPPSPLSLLLRAHFP | CATGTCCAACAAGACAACCCCTCCATCTCCTC |
| | GLKFESQDYKIAGKK | TGAGCCTGCTGCTGAGAGCCCACTTTCCTGGC |
| | LRDGGPEAVISYLTG | CTGAAGTTCGAGAGCCAGGACTACAAGATCGC |
| | KGQAKLKDVKPPAK | CGGCAAGAAACTGAGAGATGGCGGACCTGAG |
| | AFVIAQSRPFIEWDLV | GCCGTGATCAGCTACCTGACTGGAAAAGGCCA |
| | RVSRQIQEKIFGIPATK | GGCCAAGCTGAAGGACGTGAAGCCTCCTGCCA |
| | GRPKQDGLSETAFNE | AGGCCTTTGTGATCGCCCAGAGCAGACCCTTC |
| | AVASLEVDGKSKLNE | ATCGAGTGGGACCTCGTCAGAGTGTCCCGGCA |
| | ETRAAFYEVLGLDAP | GATCCAAGAGAAGATCTTTGGCATCCCCGCCA |
| | SLHAQAQNALIKSAIS | CCAAGGGCAGACCTAAGCAAGATGGCCTGAG |
| | IREGVLKKVENRNEK | CGAGACAGCCTTCAACGAAGCCGTGGCCAGCC |
| | NLSKTKRRKEAGEEA | TGGAAGTGGACGGCAAGAGCAAGCTGAACGA |
| | TFVEEKAHDERGYLI | GGAAACCAGAGCCGCCTTCTACGAGGTGCTGG |
| | HPPGVNQTIPGYQAV | GACTTGATGCCCCAAGCCTGCATGCTCAGGCC |
| | VIKSCPSDFIGLPSGCL | CAGAATGCCCTGATCAAGAGCGCCATCAGCAT |
| | AKESAEALTDYLPHD | CAGAGAAGGCGTGCTGAAGAAGGTGGAAAAC |
| | RMTIPKGQPGYVPEW | CGGAACGAGAAGAACCTGAGCAAGACCAAGC |
| | QHPLLNRRKNRRRRD | GGCGGAAAGAGGCTGGCGAAGAGGCCACCTT |
| | WYSASLNKPKATCSK | TGTGGAAGAGAAGGCCCACGACGAGCGGGGC |
| | RSGTPNRKNSRTDQIQ | TATCTGATTCATCCTCCTGGCGTGAACCAGAC |
| | SGRFKGAIPVLMRFQ | AATCCCCGGCTATCAGGCCGTGGTCATCAAGA |
| | DEWVIIDIRGLLRNAR | GCTGCCCCAGCGATTTCATCGGCCTGCCTAGT |
| | YRKLLKEKSTIPDLLS | GGCTGTCTGGCCAAAGAGTCTGCCGAGGCTCT |
| | LFTGDPSIDMRQGVC | GACCGATTACCTGCCTCACGACCGGATGACTA |
| | TFIYKAGQACSAKMV | TCCCCAAGGGACAGCCTGGCTATGTGCCCGAA |
| | KTKNAPEILSELTKSG | TGGCAGCACCCTCTGCTGAACAGAAGAAAGA |
| | PVVLVSIDLGQTNPIA | ACCGGCGCAGAAGAGACTGGTACAGCGCCAG |
| | AKVSRVTQLSDGQLS | CCTGAACAAGCCCAAGGCCACCTGTAGCAAGA |
| | HETLLRELLSNDSSDG | GATCCGGCACACCCAACCGGAAGAACAGCAG |
| | KEIARYRVASDRLRD | AACCGACCAGATCCAGAGCGGCAGATTCAAG |
| | KLANLAVERLSPEHK | GGCGCCATTCCTGTGCTGATGCGGTTCCAGGA |
| | SEILRAKNDTPALCKA | TGAGTGGGTCATCATCGACATCCGGGGCCTGC |
| | RVCAALGLNPEMIAW | TGAGAAACGCCCGGTATCGGAAGCTGCTGAAA |
| | DKMTPYTEFLATAYL | GAGAAGTCCACCATTCCTGACCTGCTGAGCCT |
| | EKGGDRKVATLKPKN | GTTCACCGGCGATCCCAGCATCGATATGAGAC |
| | RPEMLRRDIKFKGTE | AGGGCGTGTGCACCTTCATCTACAAGGCCGGC |
| | GVRIEVSPEAAEAYRE | CAGGCCTGTAGCGCCAAGATGGTCAAGACAA |
| | AQWDLQRTSPEYLRL | AGAACGCCCCTGAGATCCTGTCCGAGCTGACC |
| | STWKQELTKRILNQL | AAGTCTGGACCTGTGGTGCTGGTGTCCATCGA |
| | RHKAAKSSQCEVVV | CCTGGGCCAGACAAATCCTATCGCCGCCAAGG |
| | MAFEDLNIKMMHGN | TGTCCAGAGTGACCCAGCTGTCTGATGGCCAG |
| | GKWADGGWDAFFIK | CTGAGCCACGAGACACTGCTGAGGGAACTGCT |
| | KRENRWFMQAFHKS | GAGCAACGATAGCAGCGACGGCAAAGAGATC |
| | LTELGAHKGVPTIEVT | GCCCGGTACAGAGTGGCCAGCGACAGACTGA |
| | PHRTSITCTKCGHCDK | GAGACAAGCTGGCCAATCTGGCCGTGGAAAG |
| | ANRDGERFACQKCGF | ACTGAGCCCTGAGCACAAGAGCGAGATCCTGA |
| | VAHADLEIATDNIERV | GAGCCAAGAACGACACCCCTGCTCTGTGCAAG |
| | ALTGKPMPKPESERS | GCCAGAGTGTGTGCTGCCCTGGGACTGAACCC |
| | GDAKKSVGARKAAF | TGAAATGATCGCCTGGGACAAGATGACCCCTT |
| | KPEEDAEAAE (SEQ | ACACCGAGTTTCTGGCCACCGCCTACCTGGAA |
| | ID NO: 2468) | AAAGGCGGCGACAGAAAAGTGGCCACACTGA |
| | | AGCCCAAGAACAGACCCGAGATGCTGCGGCG |
| | | GGACATCAAGTTCAAGGGAACCGAGGGCGTC |
| | | AGAATCGAGGTGTCACCTGAAGCCGCCGAGGC |
| | | CTATAGAGAAGCCCAGTGGGATCTGCAGAGG |
| | | ACAAGCCCCGAGTACCTGAGACTGTCCACCTG |
| | | GAAGCAAGAGCTGACAAAGAGAATCCTGAAC |
| | | CAGCTGCGGCACAAGGCCGCCAAAAGCAGCC |
| | | AGTGTGAAGTGGTGGTCATGGCCTTCGAGGAC |
| | | CTGAACATCAAGATGATGCACGGCAACGGCA |
| | | AGTGGGCCGATGGTGGATGGGATGCCTTCTTC |
| | | ATCAAGAAACGCGAGAACCGGTGGTTCATGCA |
| | | GGCCTTCCACAAGAGCCTGACAGAGCTGGGAG |
| | | CACACAAGGGCGTGCCAACCATCGAAGTGACC |
| | | CCTCACAGAACCAGCATCACCTGTACCAAGTG |
| | | CGGCCACTGCGACAAGGCCAACAGAGATGGG |
| | | GAGAGATTCGCCTGCCAGAAATGCGGCTTTGT |
| | | GGCCCACGCCGATCTGGAAATCGCCACCGACA |
| | | ACATCGAGAGAGTGGCCCTGACAGGCAAGCC |
| | | CATGCCTAAGCCTGAGAGCGAGAGAAGCGGC |
| | | GACGCCAAGAAATCTGTGGGAGCCAGAAAGG |
| | | CCGCCTTCAAGCCTGAGGAAGATGCCGAAGCT |
| | | GCCGAG (SEQ ID NO: 1407) |
| CasΦ.12 | MIKPTVSQFLTPGFKL | ATGATCAAGCCTACCGTCAGCCAGTTTCTGAC |
| | IRNHSRTAGLKLKNE | CCCTGGCTTCAAGCTGATCCGGAACCACTCTA |
| | GEEACKKFVRENEIPK | GAACAGCCGGCCTGAAGCTGAAGAACGAGGG |
| | DECPNFQGGPAIANII | CGAAGAGGCCTGCAAGAAATTCGTGCGCGAG |
| | AKSREFTEWEIYQSSL | AACGAGATCCCCAAGGACGAGTGCCCCAACTT |
| | AIQEVIFTLPKDKLPEP | TCAAGGCGGACCCGCCATTGCCAACATCATTG |
| | ILKEEWRAQWLSEHG | CCAAGAGCCGCGAGTTCACCGAGTGGGAGATC |
| | LDTVPYKEAAGLNLII | TACCAGTCTAGCCTGGCCATCCAAGAAGTGAT |
| | KNAVNTYKGVQVKV | CTTCACCCTGCCTAAGGACAAGCTGCCCGAGC |
| | DNKNKNNLAKINRKN | CTATCCTGAAAGAGGAATGGCGAGCCCAGTGG |
| | EIAKLNGEQEISFEEIK | CTGTCTGAGCACGGACTGGATACCGTGCCTTA |
| | AFDDKGYLLQKPSPN | CAAAGAAGCCGCCGGACTGAACCTGATCATCA |
| | KSIYCYQSVSPKPFITS | AGAACGCCGTGAACACCTACAAGGGCGTGCA |
| | KYHNVNLPEEYIGYY | AGTGAAGGTGGACAACAAGAACAAAAACAAC |
| | RKSNEPIVSPYQFDRL | CTGGCCAAGATCAACCGGAAGAATGAGATCG |
| | RIPIGEPGYVPKWQYT | CCAAGCTGAACGGCGAGCAAGAGATCAGCTTC |
| | FLSKKENKRRKLSKRI | GAGGAAATCAAGGCCTTCGACGACAAGGGCT |
| | KNVSPILGIICIKKDW | ACCTGCTGCAGAAGCCCTCTCCAAACAAGAGC |
| | CVFDMRGLLRTNHW | ATCTACTGCTACCAGAGCGTGTCCCCTAAGCC |
| | KKYHKPTDSINDLFD | TTTCATCACCAGCAAGTACCACAACGTGAACC |
| | YFTGDPVIDTKANVV | TGCCTGAAGAGTACATCGGCTACTACCGGAAG |
| | RFRYKMENGIVNYKP | TCCAACGAGCCCATCGTGTCCCCATACCAGTT |
| | VREKKGKELLENICD | CGACAGACTGCGGATCCCTATCGGCGAGCCTG |
| | QNGSCKLATVDVGQ | GCTATGTGCCTAAGTGGCAGTACACCTTCCTG |
| | NNPVAIGLFELKKVN | AGCAAGAAAGAGAACAAGCGGCGGAAGCTGA |
| | GELTKTLISRHPTPIDF | GCAAGCGGATCAAGAATGTGTCCCCAATCCTG |
| | CNKITAYRERYDKLE | GGCATCATCTGCATCAAGAAAGATTGGTGCGT |
| | SSIKLDAIKQLTSEQKI | GTTCGACATGCGGGGCCTGCTGAGAACAAACC |
| | EVDNYNNNFTPQNTK | ACTGGAAGAAGTATCACAAGCCCACCGACAG |
| | QIVCSKLNINPNDLPW | CATCAACGACCTGTTCGACTACTTCACCGGCG |
| | DKMISGTHFISEKAQV | ATCCCGTGATCGACACCAAGGCCAATGTCGTG |
| | SNKSEIYFTSTDKGKT | CGGTTCCGGTACAAGATGGAAAACGGCATCGT |
| | KDVMKSDYKWFQDY | GAACTACAAGCCCGTGCGGGAAAAGAAGGGC |
| | KPKLSKEVRDALSDIE | AAAGAGCTGCTGGAAAACATCTGCGACCAGA |
| | WRLRRESLEFNKLSK | ACGGCAGCTGCAAGCTGGCCACAGTGGATGTG |
| | SREQDARQLANWISS | GGCCAGAACAACCCTGTGGCCATCGGCCTGTT |
| | MCDVIGIENLVKKNN | CGAGCTGAAAAAAGTGAACGGGGAGCTGACC |
| | FFGGSGKREPGWDNF | AAGACACTGATCAGCAGACACCCCACACCTAT |
| | YKPKKENRWWINAIH | CGATTTCTGCAACAAGATCACCGCCTACCGCG |
| | KALTELSQNKGKRVI | AGAGATACGACAAGCTGGAAAGCAGCATCAA |
| | LLPAMRTSITCPKCKY | GCTGGACGCCATCAAGCAGCTGACCAGCGAGC |
| | CDSKNRNGEKFNCLK | AGAAAATCGAAGTGGACAACTACAACAACAA |
| | CGIELNADIDVATENL | CTTCACGCCCCAGAACACCAAGCAGATCGTGT |
| | ATVAITAQSMPKPTC | GCAGCAAGCTGAATATCAACCCCAACGATCTG |
| | ERSGDAKKPVRARKA | CCCTGGGACAAGATGATCAGCGGCACCCACTT |
| | KAPEFHDKLAPSYTV | CATCAGCGAGAAGGCCCAGGTGTCCAACAAG |
| | VLREAV (SEQ ID NO: | AGCGAGATCTACTTTACCAGCACCGATAAGGG |
| | 12) | CAAGACCAAGGACGTGATGAAGTCCGACTAC |
| | | AAGTGGTTCCAGGACTATAAGCCCAAGCTGTC |
| | | CAAAGAAGTGCGGGACGCCCTGAGCGATATTG |
| | | AGTGGCGGCTGAGAAGAGAGAGCCTGGAATT |
| | | CAACAAGCTCAGCAAGAGCAGAGAGCAGGAC |
| | | GCCAGACAGCTGGCCAATTGGATCAGCAGCAT |
| | | GTGCGACGTGATCGGCATCGAGAACCTGGTCA |
| | | AGAAGAACAACTTCTTCGGCGGCAGCGGCAA |
| | | GAGAGAACCCGGCTGGGACAACTTCTACAAGC |
| | | CGAAGAAAGAAAACCGGTGGTGGATCAACGC |
| | | CATCCACAAGGCCCTGACAGAGCTGTCCCAGA |
| | | ACAAGGGAAAGAGAGTGATCCTGCTGCCTGCC |
| | | ATGCGGACCAGCATCACCTGTCCTAAGTGCAA |
| | | GTACTGCGACAGCAAGAACCGCAACGGCGAG |
| | | AAGTTCAATTGCCTGAAGTGTGGCATTGAGCT |
| | | GAACGCCGACATCGACGTGGCCACCGAAAATC |
| | | TGGCTACCGTGGCCATCACAGCCCAGAGCATG |
| | | CCTAAGCCAACCTGCGAGAGAAGCGGCGACG |
| | | CCAAGAAACCTGTGCGGGCCAGAAAAGCCAA |
| | | GGCTCCCGAGTTCCACGATAAGCTGGCCCCTA |
| | | GCTACACCGTGGTGCTGAGAGAAGCTGTG |
| | | (SEQ ID NO: 1408) |
| CasΦ.17 | MYSLEMADLKSEPSL | ATGTACAGCCTGGAAATGGCCGACCTGAAGTC |
| | LAKLLRDRFPGKYWL | CGAGCCTTCTCTGCTGGCTAAGCTGCTGAGAG |
| | PKYWKLAEKKRLTG | ACAGATTCCCCGGCAAGTACTGGCTGCCTAAG |
| | GEEAACEYMADKQL | TACTGGAAGCTGGCCGAGAAGAAGAGACTGA |
| | DSPPPNFRPPARCVIL | CAGGCGGAGAAGAAGCCGCCTGCGAGTACAT |
| | AKSRPFEDWPVHRVA | GGCTGACAAGCAGCTGGATAGCCCTCCACCTA |
| | SKAQSFVIGLSEQGFA | ACTTCCGGCCTCCAGCCAGATGTGTGATCCTG |
| | ALRAAPPSTADARRD | GCCAAGAGCAGACCCTTCGAGGATTGGCCAGT |
| | WLRSHGASEDDLMA | GCACAGAGTGGCCAGCAAGGCCCAGTCTTTTG |
| | LEAQLLETIMGNAISL | TGATCGGCCTGAGCGAGCAGGGCTTCGCTGCT |
| | HGGVLKKIDNANVK | CTTAGAGCTGCCCCTCCTAGCACAGCCGACGC |
| | AAKRLSGRNEARLNK | CAGAAGAGATTGGCTGAGAAGCCATGGCGCC |
| | GLQELPPEQEGSAYG | AGCGAGGATGATCTGATGGCTCTGGAAGCCCA |
| | ADGLLVNPPGLNLNI | GCTGCTGGAAACCATCATGGGCAACGCCATTT |
| | YCRKSCCPKPVKNTA | CTCTGCACGGCGGCGTGCTGAAGAAGATCGAC |
| | RFVGHYPGYLRDSDSI | AACGCCAACGTGAAGGCCGCCAAGAGACTGT |
| | LISGTMDRLTIIEGMP | CCGGAAGAAACGAGGCCAGACTGAACAAGGG |
| | GHIPAWQREQGLVKP | CCTGCAAGAGCTGCCTCCTGAGCAAGAGGGAT |
| | GGRRRRLSGSESNMR | CTGCCTATGGCGCCGATGGCCTGCTGGTTAAT |
| | QKVDPSTGPRRSTRS | CCTCCTGGCCTGAACCTGAACATCTACTGCAG |
| | GTVNRSNQRTGRNGD | AAAGAGCTGCTGCCCCAAGCCTGTGAAGAACA |
| | PLLVEIRMKEDWVLL | CCGCCAGATTCGTGGGACACTACCCCGGCTAC |
| | DARGLLRNLRWRESK | CTGAGAGACTCCGACAGCATCCTGATCAGCGG |
| | RGLSCDHEDLSLSGLL | CACCATGGACCGGCTGACAATCATCGAGGGAA |
| | ALFSGDPVIDPVRNEV | TGCCCGGACACATCCCCGCCTGGCAACGAGAA |
| | VFLYGEGIIPVRSTKP | CAGGGACTTGTGAAACCTGGCGGCAGAAGGC |
| | VGTRQSKKLLERQAS | GGAGACTGTCTGGCAGCGAGAGCAACATGAG |
| | MGPLTLISCDLGQTNL | ACAGAAGGTGGACCCCAGCACAGGCCCCAGA |
| | IAGRASAISLTHGSLG | AGAAGCACAAGATCCGGCACCGTGAACAGAA |
| | VRSSVRIELDPELIKSF | GCAACCAGCGGACAGGCAGAAACGGCGATCC |
| | ERLRKDADRLETEILT | TCTGCTGGTGGAAATCCGGATGAAGGAAGATT |
| | AAKETLSDEQRGEVN | GGGTCCTGCTGGACGCCAGAGGCCTGCTGAGA |
| | SHEKDSPQTAKASLC | AATCTGAGATGGCGCGAGTCCAAGAGAGGCCT |
| | RELGLHPPSLPWGQM | GAGCTGCGATCACGAGGATCTGAGCCTGTCTG |
| | GPSTTFIADMLISHGR | GACTGCTGGCCCTGTTTTCTGGCGACCCCGTG |
| | DDDAFLSHGEFPTLE | ATCGATCCTGTGCGGAATGAGGTGGTGTTCCT |
| | KRKKFDKRFCLESRP | GTACGGCGAGGGCATCATTCCAGTGCGGAGCA |
| | LLSSETRKALNESLW | CAAAGCCTGTGGGCACCAGACAGAGCAAGAA |
| | EVKRTSSEYARLSQR | ACTGCTGGAACGGCAGGCCAGCATGGGCCCTC |
| | KKEMARRAVNFVVEI | TGACACTGATCTCTTGTGACCTGGGCCAGACC |
| | SRRKTGLSNVIVNIED | AACCTGATTGCCGGCAGAGCCTCTGCTATCAG |
| | LNVRIFHGGGKQAPG | CCTGACACATGGATCTCTGGGCGTCAGATCCA |
| | WDGFFRPKSENRWFI | GCGTGCGGATTGAGCTGGACCCCGAGATCATC |
| | QAIHKAFSDLAAHHG | AAGAGCTTCGAGCGGCTGAGAAAGGACGCCG |
| | IPVIESDPORTSMTCPE | ACAGACTGGAAACCGAGATCCTGACCGCCGCC |
| | CGHCDSKNRNGVRFL | AAAGAAACCCTGAGCGACGAACAGAGGGGCG |
| | CKGCGASMDADFDA | AAGTGAACAGCCACGAGAAGGATAGCCCACA |
| | ACRNLERVALTGKPM | GACAGCCAAGGCCAGCCTGTGTAGAGAGCTG |
| | PKPSTSCERLLSATTG | GGACTGCACCCTCCATCTCTGCCTTGGGGACA |
| | KVCSDHSLSHDAIEK | GATGGGCCCTAGCACCACCTTTATCGCCGACA |
| | AS (SEQ ID NO: 17) | TGCTGATCTCCCACGGCAGGGACGATGATGCC |
| | | TTTCTGAGCCACGGCGAGTTCCCCACACTGGA |
| | | AAAGCGGAAGAAGTTCGATAAGCGGTTCTGCC |
| | | TGGAAAGCAGACCCCTGCTGAGCAGCGAGAC |
| | | AAGAAAGGCCCTGAACGAGTCCCTGTGGGAA |
| | | GTGAAGAGAACCAGCAGCGAGTACGCCCGGC |
| | | TGAGCCAGAGAAAGAAAGAGATGGCTAGACG |
| | | GGCCGTGAACTTCGTGGTCGAGATCTCCAGAA |
| | | GAAAGACCGGCCTGTCCAACGTGATCGTGAAC |
| | | ATCGAGGACCTGAACGTGCGGATCTTTCACGG |
| | | CGGAGGAAAACAGGCTCCTGGCTGGGATGGCT |
| | | TCTTCAGACCCAAGTCCGAGAACCGGTGGTTC |
| | | ATCCAGGCCATCCACAAGGCCTTCAGCGATCT |
| | | GGCCGCTCACCACGGAATCCCTGTGATCGAGA |
| | | GCGACCCTCAGCGGACCAGCATGACCTGTCCT |
| | | GAGTGTGGCCACTGCGACAGCAAGAACCGGA |
| | | ATGGCGTTCGGTTCCTGTGCAAAGGCTGTGGC |
| | | GCCTCCATGGACGCCGATTTTGATGCCGCCTG |
| | | CCGGAACCTGGAAAGAGTGGCTCTGACAGGC |
| | | AAGCCCATGCCTAAGCCTAGCACCTCCTGTGA |
| | | AAGACTGCTGAGCGCCACCACCGGCAAAGTGT |
| | | GCTCTGATCACTCCCTGTCTCACGACGCCATCG |
| | | AGAAGGCTTCTTAA (SEQ ID NO: 1409) |
| CasΦ.18 | MEKEITELTKIRREFP | ATGGAAAAAGAGATCACCGAGCTGACCAAGA |
| | NKKFSSTDMKKAGKL | TCCGCAGAGAGTTCCCCAACAAGAAGTTCAGC |
| | LKAEGPDAVRDFLNS | AGCACCGACATGAAGAAGGCCGGCAAGCTGC |
| | CQEIIGDFKPPVKTNI | TGAAGGCCGAAGGACCTGATGCCGTGCGGGA |
| | VSISRPFEEWPVSMVG | CTTCCTGAACAGCTGCCAAGAGATCATCGGCG |
| | RAIQEYYFSLTKEELE | ACTTCAAGCCTCCAGTCAAGACCAACATCGTG |
| | SVHPGTSSEDHKSFFN | TCCATCAGCAGACCCTTCGAGGAATGGCCCGT |
| | ITGLSNYNYTSVQGL | GTCCATGGTTGGACGGGCCATCCAAGAGTACT |
| | NLIFKNAKAIYDGTLV | ACTTCAGCCTGACCAAAGAGGAACTGGAAAG |
| | KANNKNKKLEKKFN | CGTTCACCCCGGCACCAGCAGCGAGGACCACA |
| | EINHKRSLEGLPIITPD | AGAGCTTTTTCAACATCACCGGCCTGAGCAAC |
| | FEEPFDENGHLNNPPG | TACAACTACACCAGCGTGCAGGGCCTGAACCT |
| | INRNIYGYQGCAAKV | GATCTTCAAGAACGCCAAGGCCATCTACGACG |
| | FVPSKHKMVSLPKEY | GCACCCTGGTCAAGGCCAACAACAAGAACAA |
| | EGYNRDPNLSLAGFR | GAAGCTCGAGAAGAAGTTTAACGAGATCAAC |
| | NRLEIPEGEPGHVPWF | CACAAGCGGAGCCTGGAAGGCCTGCCTATCAT |
| | QRMDIPEGQIGHVNKI | CACCCCTGATTTCGAGGAACCCTTCGACGAGA |
| | QRFNFVHGKNSGKVK | ACGGCCACCTGAACAACCCTCCAGGCATCAAC |
| | FSDKTGRVKRYHHSK | CGGAACATCTACGGCTATCAGGGCTGCGCCGC |
| | YKDATKPYKFLEESK | CAAGGTGTTCGTGCCTTCTAAGCACAAGATGG |
| | KVSALDSILAIITIGDD | TGTCCCTGCCTAAAGAGTACGAGGGCTACAAC |
| | WVVFDIRGLYRNVFY | AGGGACCCCAACCTGTCTCTGGCCGGCTTCAG |
| | RELAQKGLTAVQLLD | AAACAGACTGGAAATCCCTGAGGGCGAGCCT |
| | LFTGDPVIDPKKGVV | GGCCATGTGCCATGGTTCCAGAGAATGGATAT |
| | TFSYKEGVVPVFSQKI | CCCCGAGGGCCAGATCGGACACGTGAACAAG |
| | VPRFKSRDTLEKLTSQ | ATCCAGCGGTTCAACTTCGTGCACGGCAAGAA |
| | GPVALLSVDLGQNEP | CAGCGGCAAAGTGAAGTTCTCCGACAAGACCG |
| | VAARVCSLKNINDKIT | GCAGAGTGAAGAGATACCACCACAGCAAGTA |
| | LDNSCRISFLDDYKK | CAAGGACGCTACCAAGCCTTACAAGTTCCTGG |
| | QIKDYRDSLDELEIKI | AAGAGTCCAAGAAGGTGTCAGCCCTGGACAG |
| | RLEAINSLETNQQVEI | CATCCTGGCCATCATCACAATCGGCGACGACT |
| | RDLDVFSADRAKANT | GGGTCGTGTTCGACATCAGAGGCCTGTACCGG |
| | VDMFDIDPNLISWDS | AACGTGTTCTACAGAGAGCTGGCCCAGAAAGG |
| | MSDARVSTQISDLYL | CCTGACAGCTGTGCAACTGCTGGACCTGTTTA |
| | KNGGDESRVYFEINN | CCGGCGATCCCGTGATCGACCCCAAGAAAGGC |
| | KRIKRSDYNISQLVRP | GTGGTCACCTTCAGCTACAAAGAGGGCGTCGT |
| | KLSDSTRKNLNDSIW | CCCCGTCTTTAGCCAGAAAATCGTGCCCCGGT |
| | KLKRTSEEYLKLSKR | TCAAGAGCCGGGACACCCTGGAAAAGCTGAC |
| | KLELSRAVVNYTIRQS | CTCTCAGGGACCTGTGGCTCTGCTGTCTGTGG |
| | KLLSGINDIVIILEDLD | ACCTGGGACAGAATGAACCTGTGGCCGCCAGA |
| | VKKKFNGRGIRDIGW | GTGTGCAGCCTGAAGAACATCAACGACAAGAT |
| | DNFFSSRKENRWFIPA | CACCCTGGACAACTCTTGCCGGATCAGCTTCC |
| | FHKTFSELSSNRGLCV | TGGACGACTACAAGAAGCAGATCAAGGACTA |
| | IEVNPAWTSATCPDC | CAGAGACAGCCTGGACGAGCTGGAAATCAAG |
| | GFCSKENRDGINFTCR | ATCCGGCTGGAAGCCATCAACTCCCTCGAGAC |
| | KCGVSYHADIDVATL | AAACCAGCAGGTCGAGATCAGAGATCTGGAC |
| | NIARVAVLGKPMSGP | GTGTTCAGCGCCGACCGGGCCAAAGCCAATAC |
| | ADRERLGDTKKPRVA | CGTGGACATGTTTGACATCGACCCTAACCTGA |
| | RSRKTMKRKDISNST | TCAGCTGGGACTCCATGAGCGACGCCAGAGTC |
| | VEAMVTA (SEQ ID | AGCACCCAGATCAGCGACCTGTACCTGAAGAA |
| | NO: 18) | TGGCGGCGACGAGAGCCGGGTGTACTTTGAGA |
| | | TTAACAACAAACGGATTAAGCGGAGCGACTAC |
| | | AACATCAGCCAGCTCGTGCGGCCCAAGCTGAG |
| | | CGATAGCACCAGAAAGAACCTGAACGACAGC |
| | | ATCTGGAAGCTGAAGCGGACCAGCGAGGAAT |
| | | ACCTGAAGCTGAGCAAGCGGAAGCTGGAACT |
| | | GAGCAGAGCCGTCGTGAATTACACCATCCGGC |
| | | AGAGCAAACTGCTGAGCGGCATCAATGACATC |
| | | GTGATCATTCTCGAGGACCTGGACGTGAAGAA |
| | | GAAATTCAACGGCAGAGGCATCCGCGATATCG |
| | | GCTGGGACAACTTCTTCAGCTCCCGGAAAGAA |
| | | AACCGGTGGTTCATCCCCGCCTTCCACAAGAC |
| | | CTTTAGCGAGCTGAGCAGCAACAGGGGCCTGT |
| | | GCGTGATCGAAGTGAATCCTGCCTGGACCAGC |
| | | GCCACCTGTCCTGATTGTGGCTTCTGCAGCAA |
| | | AGAAAACAGAGATGGCATCAACTTCACGTGCC |
| | | GGAAGTGCGGCGTGTCCTACCACGCCGATATT |
| | | GACGTGGCCACACTGAATATTGCCAGAGTGGC |
| | | CGTGCTGGGCAAGCCTATGTCTGGACCTGCCG |
| | | ACAGAGAGAGACTGGGCGACACCAAGAAACC |
| | | TAGAGTGGCCCGCAGCAGAAAGACCATGAAG |
| | | CGGAAGGACATCAGCAACAGCACCGTCGAGG |
| | | CCATGGTTACAGCTTAA (SEQ ID NO: 1410) |

### Example 2

### Illustrative CasΦ Guide RNA Sequences

Guide RNA sequences for complexing with the CasΦ polypeptides of the disclosure were prepared. TABLE 5 provides illustrative guide RNA sequences to target the target nucleic acid sequence TATTAAATACTCGTATTGCTGTTCGATTAT (SEQ ID NO: 1411). A guide nucleic acid of the disclosure can comprise the sequence of any of the guide RNAs provided in Table 5 or a portion thereof.

TABLE 5
Illustrative CasΦ guide RNA sequences

| | RNA | Repeat | Spacer | RNA sequence (5′ → 3′), shown as DNA |
|---|---|---|---|---|
| Name | Type | length | length | BOLD = spacer |
| CasΦ.2 | crRNA | 36 | 30 | GTCGGAACGCTCAACGATTGCCCCTCACGAGG |
| | | | | GGAC (SEQ ID NO: 49) |
| CasΦ.7 | crRNA | 36 | 30 | GGATCCAATCCTTTTTGATTGCCCAATTCGTTG |
| | | | | GGAC (SEQ ID NO: 51) |
| CasΦ.10 | crRNA | 36 | 30 | GGATCTGAGGATCATTATTGCTCGTTACGACGA |
| | | | | GAC (SEQ ID NO: 52) |
| CasΦ.18 | crRNA | 36 | 30 | ACCAAAACGACTATTGATTGCCCAGTACGCTGG |
| | | | | GAC (SEQ ID NO: 57) |

### Example 3

### CasΦ Acts as a Programmable Nickase

The present example shows that a CasΦ polypeptide can comprise programmable nickase activity. FIG. 1 shows data from an experiment to analyze nicking ability of CasΦ ortholog proteins. For this experiment, five different CasΦ polypeptides: designated CasΦ.2, CasΦ.11, CasΦ.17, CasΦ.18, and CasΦ.12 in FIG. 1, were analyzed. Amino acid sequences of the proteins used in the experiment are shown in TABLE 4.

All reactions were carried out using guide RNA comprising a crRNA sequence comprising the CasΦ.18 repeat sequence (ACCAAAACGACTATTGATTGCCCAGTACGCTGGGAC (SEQ ID NO: 57)). Complexing of the CasΦ polypeptide with a guide RNA to form the ribonucleoprotein (RNP) complex was carried out at room temperature for 20 minutes. The RNP complex was incubated with the target DNA at 37° C. for 60 minutes in NEB CutSmart buffer (50 mM Potassium Acetate, 20 mM Tris-Acetate, 10 mM Magnesium Acetate, 100 ug/ml BSA, pH 7.9 at 25° C.). The target nucleic acid used for the reactions was a super-coiled plasmid DNA comprising the target sequence TATTAAATACTCGTATTGCTGTTCGATTAT (SEQ ID NO: 116), which was immediately downstream of a TTTN PAM sequence. The plasmid DNA sequence is provided below with the target sequence in bold:

(SEQ ID NO: 1412)
gtgtagataactacgatacgggagggcttaccatctggccccagtgctgc
aatgataccgcgagacccacgctcaccggctccagatttatcagcaataa
accagccagccggaagggccgagcgcagaagtggtcctgcaactttatcc
gcctccatccagtctattaattgttgccgggaagctagagtaagtagttc
gccagttaatagtttgcgcaacgttgttgccattgctacaggcatcgtgg
tgtcacgctcgtcgtttggtatggcttcattcagctccggttcccaacga
tcaaggcgagttacatgatcccccatgttgtgcaaaaaagcggttagctc
cttcggtcctccgatcgttgtcagaagtaagttggccgcagtgttatcac
tcatggttatggcagcactgcataattctcttactgtcatgccatccgta
agatgcttttctgtgactggtgagtactcaaccaagtcattctgagaata
gtgtatgcggcgaccgagttgctcttgcccggcgtcaatacgggataata
ccgcgccacatagcagaactttaaaagtgctcatcattggaaaacgttct
tcggggcgaaaactctcaaggatcttaccgctgttgagatccagttcgat
gtaacccactcgtgcacccaactgatcttcagcatcttttactttcacca
gcgtttctgggtgagcaaaaacaggaaggcaaaatgccgcaaaaaaggga
ataagggcgacacggaaatgttgaatactcatactcttcctttttcaata
ttattgaagcatttatcagggttattgtctcatgagcggatacatatttg
aatgtatttagaaaaataaacaaataggggttccgcgcacatttccccga
aaagtgccacctgacgtctaagaaaccattattatcatgacattaaccta
taaaaataggcgtatcacgaggccctttcgtctcgcgcgtttcggtgatg
acggtgaaaacctctgacacatgcagctcccggagacggtcacagcttgt
ctgccatggacatgtttaTATTAAATACTCGTATTGCTGTTCGATTATga
ccgaattccctgtcgtgccagctgcattaatgaatcggccaacgcgcggg
gagaggcggtttgcgtattgggcgctcttccgcttcctcgctcactgact
cgctgcgctcggtcgttcggctgcggcgagcggtatcagctcactcaaag
gcggtaatacggttatccacagaatcaggggataacgcaggaaagaacat
gtgagcaaaaggccagcaaaaggccaggaaccgtaaaaaggccgcgttgc
tggcgtttttccataggctccgcccccctgacgagcatcacaaaaatcga
cgctcaagtcagaggtggcgaaacccgacaggactataaagataccaggc
gtttccccctggaagctccctcgtgcgctctcctgttccgaccctgccgc
ttaccggatacctgtccgcctttctcccttcgggaagcgtggcgctttct
catagctcacgctgtaggtatctcagttcggtgtaggtcgttcgctccaa
gctgggctgtgtgcacgaaccccccgttcagcccgaccgctgcgccttat
ccggtaactatcgtcttgagtccaacccggtaagacacgacttatcgcca
ctggcagcagccactggtaacaggattagcagagcgaggtatgtaggcgg
tgctacagagttcttgaagtggtggcctaactacggctacactagaagaa
cagtatttggtatctgcgctctgctgaagccagttaccttcggaaaaaga
gttggtagctcttgatccggcaaacaaaccaccgctggtagcggtggttt
ttttgtttgcaagcagcagattacgcgcagaaaaaaaggatctcaagaag
atcctttgatcttttctacggggtctgacgctcagtggaacgaaaactca
cgttaagggattttggtcatgagattatcaaaaaggatcttcacctagat
ccttttaaattaaaaatgaagttttaaatcaatctaaagtatatatgagt
aaacttggtctgacagttaccaatgcttaatcagtgaggcacctatctca
gcgatctgtctatttcgttcatccatagttgcctgactccccgtc

As shown in FIG. 1, CasΦ.17 and CasΦ.18 produced only nicked product (i.e. single strand breaks; “nicked”) by 60 minutes. By way of comparison, CasΦ.12 generated almost entirely linearized product demonstrating double-stranded breaks, while CasΦ.2 and CasΦ.11 generated some linearized product (i.e. double strand breaks) but primarily produced nicked intermediate. This data demonstrates that CasΦ orthologs can comprise programmable nickase activity.

### Example 4

### Effect of crRNA Repeat Sequence and RNP Complexing Temperature on CasΦ Nickase Activity

The present example shows that the crRNA repeat sequence and RNP complexing temperature can affect nickase activity of CasΦ. FIG. 2A and FIG. 2B illustrate results of a cis-cleavage experiment showing the percentage of input plasmid DNA that was nicked after 60 minutes of reaction at 37° C. by CasΦ RNP complex assembled at room temperature (FIG. 2A) or at 37° C. (FIG. 2B). FIG. 2C illustrates alignment of CasΦ.2, CasΦ.7, CasΦ.10, and CasΦ.18 repeat sequences showing conserved (highlighted in black) and diverged nucleotides.

For this study, each of three CasΦ polypeptides (CasΦ.11, CasΦ.17 and CasΦ.18 in FIGS. 2A and 2B) was tested for their ability to nick input plasmid DNA when complexed with one of four crRNAs comprising the repeat sequences of CasΦ.2, CasΦ.7, CasΦ.10 and CasΦ.18 (abbreviated j2, j7, j10 and j18, respectively in FIG. 2A and FIG. 2B). Amino acid sequences of the proteins used in the experiment are shown in TABLE 4. Guide RNA sequences corresponding to j2, j7, j10 and j18 are provided in TABLE 5. The input plasmid was a super-coiled plasmid (sequence shown in EXAMPLE 3) comprising the target sequence TATTAAATACTCGTATTGCTGTTCGATTAT (SEQ ID NO: 10⁸) immediately downstream of a TTTN PAM. The incubation reaction to form the RNP complex was performed either at room temperature or at 37° C. for 60 minutes in NEB CutSmart buffer (50 mM Potassium Acetate, 20 mM Tris-Acetate, 10 mM Magnesium Acetate, 100 ug/ml BSA, pH 7.9 at 25° C.). The RNP complex was incubated with the input plasmid for 60 minutes at 37° C. The reaction was quenched with 1 mg/ml proteinase K, 0.08% SDS, and 15 mM EDTA. The data illustrated in FIG. 2A and FIG. 2B comes from a single replicate of the in vitro cis-cleavage experiment.

As shown in FIG. 2A, when the CasΦ polypeptides were assembled into RNP complexes with the guide nucleic acids at room temperature, crRNAs comprising repeat sequences from any of the proteins supported nickase activity by CasΦ.11, CasΦ.17 and CasΦ.18, with the exception of the CasΦ.17/CasΦ.2-repeat pairing. As shown in FIG. 2B, when the CasΦ polypeptides were assembled into RNP complexes with the guide nucleic acids at 37° C., as opposed to at room temperature, the activity of each protein was completely abolished when complexed with crRNAs comprising a repeat sequence from CasΦ.2 or CasΦ.10.

This example showed that the nickase activity of CasΦ can be affected by the crRNA repeat sequence. The data also showed that the nickase activity of CasΦ can be affected by the RNP complexing temperature.

FIG. 2D provides further examples of the nickase activity of CasΦ affected by the RNP complexing temperature. Nickase activity was assessed as described above for CasΦ.2, CasΦ.4, CasΦ.6, CasΦ.9, CasΦ.10, CasΦ.12 and CasΦ.13. Amino acid sequences of the proteins used in the experiment are shown in TABLE 1.

The effect of complexing temperature on the double strand cutting activity of CasΦ polypeptides was also assessed as described above. As shown in FIG. 2D, generally the double strand cutting activity of CasΦ polypeptides, particularly CasΦ.2, CasΦ.4 and CasΦ.12, is not affected by the RNP complexing temperature. Although some systems with less efficient double strand cutting activity, such as CasΦ.10, CasΦ.11 and CasΦ.13 in this example, are sensitive to RNP complexing temperature.

### Example 5

### CasΦ Nickase Cleaves Non-Target Strand

The present example shows that CasΦ nickase cleaves the non-target DNA strand. Results of the study are shown in FIG. 3. For this study, four different CasΦ polypeptides (CasΦ.12, CasΦ.2, CasΦ.11, and CasΦ.18 as shown in FIG. 1) were analyzed using a cis-cleavage assay. Amino acid sequences of the proteins used in the experiment are shown in TABLE 4. The CasΦ polypeptides were complexed with guide RNA to form RNP complexes All reactions were carried out using guide RNA comprising a crRNA sequence comprising the CasΦ.18 repeat sequence (ACCAAAACGACTATTGATTGCCCAGTACGCTGGGAC (SEQ ID NO: 57)). Complexing of the CasΦ polypeptides with guide RNA to form the ribonucleoprotein (RNP) complex was carried out at room temperature for 20 minutes. The RNP complex was incubated with the target DNA at 37° C. for 60 minutes in NEB CutSmart buffer (50 mM Potassium Acetate, 20 mM Tris-Acetate, 10 mM Magnesium Acetate, 100 ug/ml BSA, pH 7.9 at 25° C. The target nucleic acid used for the reactions was a super-coiled plasmid DNA (sequence shown in EXAMPLE 3) comprising the target sequence TATTAAATACTCGTATTGCTGTTCGATTAT (SEQ ID NO: 116), which was immediately downstream of a TTTN PAM sequence. The reaction was quenched with 1 mg/ml proteinase K, 0.08% SDS, and 15 mM EDTA. The resulting cleaved DNA from the reaction was Sanger sequenced using forward and reverse primers. The forward primer provided the sequence of the target strand (TS), while the reverse primer provided the sequence of the non-target strand (NTS). If a strand had been cleaved by the CasΦ polypeptide, the sequencing signal would drop off from the cleavage site in the sequencing data. FIG. 3 illustrates results of the Sanger sequencing.

FIG. 3, panel A, shows a control reaction where no CasΦ polypeptide was added. As a result, the target DNA was uncut and resulted in complete sequencing of both target and non-target strands. FIG. 3, panel B, illustrates the cleavage pattern for CasΦ.12, which comprises double-stranded DNA cleavage activity. The sequencing signal dropped off on both the target and the non-target strands (as shown by arrows), demonstrating cleavage of both strands of the target DNA. FIG. 3, panel C, illustrates the cleavage pattern for CasΦ.2, which predominantly nicks DNA (as illustrated in FIG. 1). The data showed that the sequencing signal dropped off on only the non-target strand (bottom arrow) demonstrating cleavage of the non-target strand. FIG. 3, panel D, illustrates the cleavage pattern for CasΦ.11, which comprises strong nickase activity (as illustrated in FIG. 1). The data showed that the sequencing signal dropped off on only the non-target strand (bottom arrow) demonstrating cleavage of the non-target strand. FIG. 3, panel E, illustrates the cleavage pattern for CasΦ.18, which comprises strong nickase activity (as illustrated in FIG. 1). The data showed that the sequencing signal dropped off on only the non-target strand (bottom arrow) demonstrating cleavage of the non-target strand. Thus, this example shows that CasΦ polypeptides comprising nickase activity cleave the non-target strand of a target DNA.

### Example 6

### Editing a Target Nucleic Acid

This example describes genetic modification of a target nucleic acid with a programmable CasΦ nuclease (e.g., any one of SEQ ID NO: 1-SEQ ID NO: 47, SEQ ID NO: 105 or SEQ ID NO: 107) of the present disclosure. The programmable CasΦ nuclease is administered with a guide nucleic acid capable of hybridizing to a segment of a target nucleic acid sequence of interests in a ribonucleoprotein complex or as separate nucleic acids encoding for each component. Subjects administered said composition are humans or non-human mammals. Upon binding of the guide nucleic acid to the segment of the target nucleic acid, the programmable CasΦ nuclease nicks or induces a double stranded break in the target. The target undergoes NHEJ or HDR. A donor nucleic acid may be co-administered. The donor nucleic acid may be to replace or repair a mutated segment of the target nucleic acid. The subject may have a disease. Upon genetic modification of the target nucleic acid, the disease or a symptom of the disease may be alleviated, or the disease may be cured.

### Example 7

### Editing a Plant or Crop Target Nucleic Acid

This example describes genetic modification of a plant or crop target nucleic acid with a programmable CasΦ nuclease (e.g., any one of SEQ ID NO: 1-SEQ ID NO: 47, SEQ ID NO: 105 or SEQ ID NO: 107) of the present disclosure. The programmable CasΦ nuclease is administered with a guide nucleic acid capable of hybridizing to a segment of a target nucleic acid sequence of interests in a ribonucleoprotein complex or as separate nucleic acids encoding for each component. Subjects administered said composition are plant or crop cells. Upon binding of the guide nucleic acid to the segment of the target nucleic acid, the programmable CasΦ nuclease nicks or induces a double stranded break in the target. The target undergoes NHEJ or HDR. A donor nucleic acid may be co-administered. The donor nucleic acid may be to replace or repair a mutated segment of the target nucleic acid. The result is an engineered plant or crop cell.

### Example 8

### Genetic Modification of a Target Nucleic Acid

This example describes genetic modification of a target nucleic acid with a dead programmable CasΦ nuclease (e.g., any one of SEQ ID NO: 1-SEQ ID NO: 47, SEQ ID NO: 105 or SEQ ID NO: 107 with a mutation rendering it catalytically inactive) of the present disclosure. The programmable CasΦ nuclease is further linked to a transcriptional regulator. The programmable CasΦ nuclease, the transcriptional regulator, and the guide nucleic acid capable of hybridizing to a segment of a target nucleic acid sequence of interests are administered as a ribonucleoprotein complex or as separate nucleic acids encoding for each component. Subjects administered said composition are humans or non-human mammals. Upon binding of the guide nucleic acid to the segment of the target nucleic acid, the dead programmable CasΦ nuclease upregulates or downregulates transcription. The subject may have a disease. Upon genetic modification of the target nucleic acid, the disease or a symptom of the disease may be alleviated, or the disease may be cured.

### Example 9

### Genetic Modification of a Plant of Crop Target Nucleic Acid

This example describes genetic modification of a plant or crop target nucleic acid with a dead programmable CasΦ nuclease (e.g., any one of SEQ ID NO: 1-SEQ ID NO: 47, SEQ ID NO: 105 or SEQ ID NO: 107 with a mutation rendering it catalytically inactive) of the present disclosure. The programmable CasΦ nuclease is further linked to a transcriptional regulator. The programmable CasΦ nuclease, the transcriptional regulator, and the guide nucleic acid capable of hybridizing to a segment of a target nucleic acid sequence of interests are administered as a ribonucleoprotein complex or as separate nucleic acids encoding for each component. Subjects administered said composition are humans or non-human mammals. Upon binding of the guide nucleic acid to the segment of the target nucleic acid, the dead programmable CasΦ nuclease upregulates or downregulates transcription. The result is an engineered plant or crop cell.

### Example 10

### Detection of a Target Nucleic Acid

This example describes detection of a target nucleic acid with a programmable CasΦ nuclease (e.g., any one of SEQ ID NO: 1-SEQ ID NO: 47, SEQ ID NO: 105 or SEQ ID NO: 107) of the present disclosure. The programmable CasΦ nuclease, the guide nucleic acid capable of hybridizing to a segment of a target nucleic acid sequence of interests, and a labeled ssDNA reporter are contacted to a sample. In the presence of the target nucleic acid in the sample, the guide nucleic acid binds to its target, thereby activating the programmable CasΦ nuclease to cleave the labeled ssDNA reporter and releasing a detectable label. The detectable label emits a detectable signal that is, optionally, quantified. In the absence of the target nucleic acid in the sample, the guide nucleic acid does not bind to its target, the labeled ssDNA reporter is not cleaved, and low or no signal is detected.

### Example 11

### Preference for Nicking or Double Strand Cleavage of Target DNA is a Property of CasΦ Enzymes, Independent of crRNA Repeat or Target Sequences

This example describes how the preference of a CasΦ polypeptide to cleave a single or both strands of a double-strand target DNA is independent of the crRNA repeat or target sequence. For this study, each of twelve CasΦ polypeptide (CasΦ.1, CasΦ.2, CasΦ.3, CasΦ.4, CasΦ.6, CasΦ.9, CasΦ.10, CasΦ.11, CasΦ.12, CasΦ.13, CasΦ.17 and CasΦ.18) was complexed with one of the crRNAs comprising the repeat sequences of CasΦ.1, CasΦ.2, CasΦ.4, CasΦ.7, CasΦ.10, CasΦ.11, CasΦ.12, CasΦ.13, CasΦ.17 and CasΦ.18. Amino acid sequences of the proteins used in the experiment are shown in TABLE 1 and crRNA sequences are provided in TABLE 2. The input plasmid was one of two super-coiled plasmids containing a target sequence (TATTAAATACTCGTATTGCTGTTCGATTAT (SEQ ID NO: 10⁸) or CACAGCTTGTCTGTAAGCGGATGCCATATG (SEQ ID NO: 109)) immediately downstream of a TTTN PAM. The incubation reaction to form the RNP complex was performed at room temperature for 20 minutes in NEB CutSmart buffer (50 mM Potassium Acetate, 20 mM Tris-Acetate, 10 mM Magnesium Acetate, 100 ug/ml BSA, pH 7.9 at 25° C.). The RNP complex was incubated with the input plasmid for 60 minutes at 37° C. The reaction was quenched with 1 mg/ml proteinase K, 0.08% SDS, and 15 mM EDTA.

As shown in FIG. 4A, CasΦ polypeptides have a preference for nicking or linearizing (i.e. cleaving both strands) a double strand plasmid DNA target and this preference is not affected by the crRNA repeat or target DNA sequence.

Raw data used to generate a subset of the heatmap in FIG. 4A is shown in FIG. 4B. These data show that CasΦ.12 is predominantly a linearizer of plasmid DNA, i.e. CasΦ.12 predominantly cleaves both strands of a double strand target DNA. Whereas CasΦ.18 is predominantly a nickase and predominantly cleaves one strand of a double strand target DNA.

This example showed that the preference of a CasΦ polypeptide to cleave a single or both strands of a double-strand target DNA is independent of the crRNA repeat or target sequence.

### Example 12

### Structural Conservation Across the CasΦ Repeats

This example describes the conservation of structure across the CasΦ repeats. In particular, FIG. 5A shows the structure of the crRNA repeats for CasΦ.1, CasΦ.2, CasΦ.7, CasΦ.11, CasΦ.12, CasΦ.13, CasΦ.18, and CasΦ.32. crRNA sequences are provided in TABLE 2. There is high sequence and structure conservation in the 3′ half of the CasΦ repeats. The LocARNA alignment tool was used to confirm the consensus structure of CasΦ repeats, which is shown in FIG. 5B. The consensus was determined on the basis of the following crRNA repeats: CasΦ.1, CasΦ.2, CasΦ.4, CasΦ.7, CasΦ.10, CasΦ.11, CasΦ.12, CasΦ.13, CasΦ.17, CasΦ.18, CasΦ.19, CasΦ.21, CasΦ.22, CasΦ.23, CasΦ.24, CasΦ.25, CasΦ.26, CasΦ.27, CasΦ.28, CasΦ.29, CasΦ.30, CasΦ.31, CasΦ.32, CasΦ.33, CasΦ.35, CasΦ.41. The sequence of these repeats is provided in TABLE 5. As shown in FIG. 5B, CasΦ repeats have a highly conserved 3′ hairpin which includes a double stranded stem portion and a single-stranded loop portion. One strand of the stem includes the sequence CYC and the other strand includes the sequence GRG, where Y and R are complementary. The loop portion typically comprises four nucleotides. The 3′ end of CasΦ repeats comprise the sequence GAC and the G of this sequence is in the stem of the hairpin.

This example shows the conserved structure of CasΦ crRNA repeats.

### Example 13

### CasΦ PAM Preferences on Linear Targets

The present example shows the PAM preferences for CasΦ polypeptides on linear double stranded DNA targets. For this study, five different CasΦ polypeptides (CasΦ.2, CasΦ.4, CasΦ.11, CasΦ.12 and CasΦ.18) were analyzed using a cis-cleavage assay. Amino acid sequences of the proteins used are shown in TABLE 1. The CasΦ polypeptides were complexed their native crRNAs (i.e. the corresponding CasΦ.2, CasΦ.4, CasΦ.11, CasΦ.12 and CasΦ.18 repeats) to form RNP complexes at room temperature for 20 minutes. The RNP complex was incubated with target DNA at 37° C. for 60 minutes in NEB CutSmart buffer (50 mM Potassium Acetate, 20 mM Tris-Acetate, 10 mM Magnesium Acetate, 100 ug/ml BSA, pH 7.9 at 25° C.). The target DNA was a 1.1 kb PCR-amplified DNA product. Stating with a TTTA PAM, each position was varied one by one to the other 3 nucleotides for a total of 12 variants in addition to the parental TTTA PAM. Linear fragments were used to disfavor cleavage for greater sensitivity of PAM preference determination. FIG. 6A illustrates the absolute levels of double strand cleavage (or nicking for CasΦ.18). FIG. 6B illustrates the data from FIG. 6A after normalization to the parental TTTA PAM as 100%. FIG. 6C provides a summary of the optimal PAM preferences from the data in FIG. 6A and FIG. 6B. CasΦ.2 recognizes a GTTK PAM, where K is G or T. CasΦ.4 recognizes a VTTK PAM, where V is A, C or G and K is G or T. CasΦ.11 recognizes a VTTS PAM, where V is A, C or G and S is C or G. CasΦ.12 recognizes a TTTS PAM, where S is C or G. CasΦ.18 recognizes a VTTN PAM, where V is A, C or G and N is A, C, G or T.

This example shows the optimized PAM preferences for some of the CasΦ polypeptides.

### Example 14

### CasΦ Polypeptides Rapidly Nick Supercoiled DNA

The present example shows that CasΦ polypeptides rapidly nick supercoiled DNA but vary in their ability to deliver the second strand cleavage. For this study, five different CasΦ polypeptides (CasΦ.2, CasΦ.4, CasΦ.11, CasΦ.12 and CasΦ.18) were analyzed using a cis-cleavage assay. Amino acid sequences of the proteins used are shown in TABLE 1. The CasΦ polypeptides were complexed with their native crRNA to form 200 nM RNP complexes at room temperature in NEB CutSmart buffer (50 mM Potassium Acetate, 20 mM Tris-Acetate, 10 mM Magnesium Acetate, 100 ug/ml BSA, pH 7.9 at 25° C.) for 20 minutes in a volume of 30 μl. The target plasmid was one of two 2.2 kb super-coiled plasmids containing a target sequence (TATTAAATACTCGTATTGCTGTTCGATTAT (SEQ ID NO: 10⁸) or CACAGCTTGTCTGTAAGCGGATGCCATATG (SEQ ID NO: 109), the guide RNAs targeted the underlined sequence) immediately downstream of a GTTG or TTTG PAM. At time “0” 30 μl of 20 nM target plasmid was mixed with RNP for a total volume of 60 μl. The incubation temperature was 37° C. At 1, 3, 6, 15, 30 and 60 minutes, 9 μl portions of the reaction were withdrawn and stopped with reaction quench (1 mg/ml proteinase K, 0.08% SDS and 15 mM EDTA) and allowed to deproteinize for 30 minutes at 37° C. before agarose gel analysis. The cleavage was quantified as nicked or linear. FIG. 7 shows the rapid nicking of supercoiled target DNA by CasΦ polypeptides. The decrease in nicked products over time is due to the formation of linear product as the CasΦ polypeptides cleaves the second strand of the target DNA. CasΦ.12 rapidly cleaves both strands of supercoiled DNA.

This example shows that CasΦ polypeptides rapidly nick supercoiled DNA.

### Example 15

### CasΦ Polypeptides Prefers Full Length Repeats and Spacers Form 16-20 Nucleotide

The present example shows that CasΦ polypeptides prefer full-length repeats and spacers from 16 to 20 nucleotides. For this study, each of five CasΦ polypeptides (CasΦ.2, CasΦ.4, CasΦ.11, CasΦ.12 and CasΦ.18 in FIGS. 8A and 8B) was tested for their ability to cleave input plasmid DNA when complexed with one of either of the crRNAs comprising the repeat sequences of CasΦ.2 or CasΦ.18 (abbreviated j2 and j18, respectively in FIG. 8A and FIG. 8B). Amino acid sequences of the proteins used in the experiment are shown in TABLE 1. Guide RNA sequences corresponding to j2 and j18 are provided in TABLE 2. The CasΦ polypeptides were complexed to the crRNA in NEB CutSmart Buffer (50 mM Potassium Acetate, 20 mM Tris-Acetate, 10 mM Magnesium Acetate, 100 ug/ml BSA, pH 7.9 at 25° C.) for 20 minutes at room temperature. The ability of the CasΦ polypeptides to cleave a 2.2 kb plasmid containing a target sequence was assessed (FUT8_1: ACGCGTTTTAGAAGAGCAGCTTGTTAAGGCCAAAGAACAGATTGA (SEQ ID NO: 1413) and DNMT_1: AAAGATTTGTCCTTGGAGAACGGTGCTCATGCTTACAACCGGGA (SEQ ID NO: 1414), the PAM is underlined). Spacers targeting these target sequences were shortened from the 3′ end. The cleavage incubation was at 37° C. and the reaction was quenched after 10 minutes with 1 mg/ml proteinase K, 0.08% SDS and 15 mM EDTA. To assess the effect of shortening the crRNA repeats, the repeats were shortened from the 5′ end.

As shown in FIG. 8A, cRNA repeats with a length of 19 to 37 nucleotides supported cleavage activity of CasΦ polypeptides.

As shown in FIG. 8B, cleavage activity was observed over the range of spacer lengths tested (16 to 35 nucleotides). The optimal spacer length to support the cleavage activity of CasΦ polypeptides in this in vitro system is 16 to 20 nucleotides.

This example shows that CasΦ polypeptides prefer crRNA repeat lengths of 19 to 37 nucleotides and spacer lengths of 16 to 20 nucleotides in vitro.

### Example 16

### CasΦ.12 Spacer Length Optimization in HEK293T Cells

The present example shows the use of CasΦ.12 as a gene editing tool in HEK293T cells and the effect of changing the length of the spacer. As illustrated in the schematic in FIG. 9A, a stable HEK293T cell line that expresses AcGFP was established. A plasmid expressing the crRNA under the control of the U6 promoter and CasΦ.12 under the control of the EF1a promoter was transfected into the AcGFP-expressing HEK293T cell line. The CasΦ.12 was expressed as FLAGtag-SV40NLS-Cas12j.12-NLS-T2A-PuroR. GFP expression was assessed by flow cytometry at days 5, 7 and 10. The 30 nucleotide spacer sequence is 5′-TTGCCCAGGATGTTGCCATCCTCCTTGAAA-3′ (SEQ ID NO: 1415). To assess the effect of different spacer length, the spacer was shortened from its 3′ end. As shown in FIG. 9B, a spacer length of 15 to 30 nucleotides supported CasΦ.12 cleavage activity in HEK293T cells, but with less cleavage detected with the 15 and 16 nucleotide spacers. There is a preference for CasΦ.12 to have a spacer length of 17 to 22 nucleotides, but cleavage activity is still supported with the longer spacers tested.

### Example 17

### CasΦ Nucleases are a Novel Class of Protein

This example illustrates that the CasΦ nucleases identified herein are a novel class of Cas proteins. SEQ ID NOs: 1 to 47 and SEQ ID NO. 105 were searched in the InterPro database, but were not identified as belonging to a class of protein. As an example, the results for SEQ ID NO: 2 are shown in FIG. 10A. As a positive control, the Cpf1 sequence from Acidaminococcus sp. (strain BV3L6) was also searched and was identified as a CRISPR-associated endonuclease Cas12a family member, as shown in FIG. 10B.

### Example 18

### DNA Cleavage by CasΦ.19-CasΦ.48

This example illustrates the DNA cleavage activity of CasΦ.19 to CasΦ.45. Amino acid sequences of the proteins used in the experiment are shown in TABLE 1. The CasΦ polypeptides were complexed with their native crRNA (or the crRNA of the CasΦ polypeptide with the closest match based on amino acid sequence identity) to form 100 nM RNP complexes at room temperature in NEB CutSmart buffer (50 mM Potassium Acetate, 20 mM Tris-Acetate, 10 mM Magnesium Acetate, 100 ug/ml BSA, pH 7.9 at 25° C.) for 20 minutes in a volume of 30 μl. crRNA sequences are provided in TABLE 2. The target plasmid was a 2.1 kb plasmid containing the target sequence TATTAAATACTCGTATTGCTGTTCGATTAT (SEQ ID NO: 10⁸). The cleavage incubation was performed at 37° C. and the reaction was quenched after 60 minutes. Cleavage products where then analyzed by gel electrophoresis, as shown in FIG. 13A. This analysis identifies CasΦ.20, CasΦ.22, CasΦ.24, CasΦ.25, CasΦ.28, CasΦ.31, CasΦ.32, CasΦ.37, CasΦ.43 and CasΦ.45 as enzymes that predominantly linearize plasmid DNA, i.e. they predominantly cleave both strands of a double strand target DNA. Whereas DNA cleavage by CasΦ.21 results in mixed nicked and linear product, indicating that CasΦ.21 functions as a nickase as well as a linearizer of plasmid DNA with a preference for nickase activity under the conditions of the present study. Mixed nicked and linearized cleavage products were also identified following cleavage by CasΦ.26, CasΦ.29, CasΦ.33, CasΦ.34, CasΦ.38 and CasΦ.44. ‘SC’ represents ‘super-coiled’ un-cut target plasmid.

This example shows robust DNA cleavage by CasΦ polypeptides.

The inventors went on to demonstrate the robust generation of indels following targeting by CasΦ.12, CasΦ.20, CasΦ.21, CasΦ.22, CasΦ.25, CasΦ.28, CasΦ.31, CasΦ.32, CasΦ.33, CasΦ.34, CasΦ.37, CasΦ.43, and CasΦ.45. A stable HEK293T cell line that expresses AcGFP was established. HEK293T-AcGFP cells were transfected with crRNA and CasΦ expression plasmids using lipofectamine on day 0. Target sequences are provided in TABLE 6. Cells were harvested by trypsinization on day 3 for TIDE analysis. The target locus was amplified by PCR and the amplified product was then sequenced using Sanger sequencing. The TIDE analysis provides the frequency of indel mutations (https://tide.nki.n1/#about). As shown in FIG. 13B, targeting CasΦ.12, CasΦ.20, CasΦ.21, CasΦ.22, CasΦ.25, CasΦ.28, CasΦ.31, CasΦ.32, CasΦ.33, CasΦ.34, CasΦ.37, CasΦ.43, and CasΦ.45 to AcGFP led to the robust generation of indel mutations. FIG. 13C provides an alternative representation of the data shown in FIG. 13B for CasΦ.12, CasΦ.28, CasΦ.31, CasΦ.32 and CasΦ.33. These data further demonstrate the genome editing ability of CasΦ.20, CasΦ.21, CasΦ.22, CasΦ.25, CasΦ.28, CasΦ.31, CasΦ.32, CasΦ.33, CasΦ.34, CasΦ.37, CasΦ.43, and CasΦ.45.

TABLE 6

| | | PAM | | SEQ ID |
|---|---|---|---|---|
| | Target Sequence | eGFP | PAM acGFP | NO |
| KT_eGFP | TTAAGGCCAAAGAACAGATT | CTTG | CTTG | 1416 |
| OT_eGFP | CGTGATGGTCTCGATTGAGT | None | None | 1417 |
| T1_eGFP | AAGAAGTCGTGCTGCTTCAT | CTTG | CTTG | 1418 |
| T2_eGFP | ATCTGCACCACCGGCAAGCT | GTTC | GTTC | 1419 |
| T3_eGFP | TGGCGGATCTTGAAGTTCAC | GTTG | GTTG | 1420 |
| T4_eGFP | CCGTAGGTGGCATCGCCCTC | GTTC | CTTC | 1421 |
| T5_eGFP | ACGTCGCCGTCCAGCTCGAC | GTTT | None | 1422 |
| T6_eGFP | AAGAAGATGGTGCGCTCCTG | CTTG | CTCG | 1423 |

### Example 19

### PAM Requirement for CasΦ Determined by In Vitro Enrichment

This example illustrates the NTTN PAM requirement for CasΦ.2, CasΦ.4, CasΦ.11 and CasΦ.12. An in vitro enrichment (IVE) analysis was performed. The CasΦ polypeptides were complexed with crRNA to form 500 nM RNP complexes at room temperature in NEB CutSmart buffer (50 mM Potassium Acetate, 20 mM Tris-Acetate, 10 mM Magnesium Acetate, 100 ug/ml BSA, pH 7.9 at 25° C.) for 30 minutes in a volume of 25 μl. crRNA sequences are provided in TABLE 2. The cleavage incubation was performed at 37° C. and the reaction was quenched after 30 minutes. The substrate for the cleavage incubation was a pooled plasmid library which includes different PAM sequences. After quenching, the cleavage reactions were cleaned using Beckman SPRi beads. The samples were sequenced to identify which PAM sequences enabled target cleavage by the CasΦ polypeptides. As shown in FIG. 14A, this analysis revealed an NTTN PAM requirement for CasΦ.2, CasΦ.4, CasΦ.11 and CasΦ.12.

The inventors went on to assess the PAM requirement of CasΦ.20, CasΦ.26, CasΦ.32, CasΦ.38 and CasΦ.45. An IVE analysis was performed using the protocol described above for CasΦ.2, CasΦ.4, CasΦ.11 and CasΦ.12. As shown in FIG. 14B, Sanger sequencing revealed a NTNN PAM requirement for CasΦ.20, a NTTG PAM requirement for CasΦ.26, a GTTN PAM requirement for CasΦ.32 and CasΦ.38, and a NTTN PAM requirement for CasΦ.45.

The inventors also determined a single-base PAM requirement for CasΦ.20, CasΦ.24 and CasΦ.25. Amino acid sequences of the proteins used are shown in TABLE 1. The CasΦ polypeptides were complexed with their native crRNAs to form RNP complexes at room temperature for 20 minutes. crRNA sequences are provided in TABLE 2. The RNP complexes were incubated with target DNA at 37° C. for 60 minutes in NEB CutSmart buffer (50 mM Potassium Acetate, 20 mM Tris-Acetate, 10 mM Magnesium Acetate, 100 ug/ml BSA, pH 7.9 at 25° C.). The RNPs were then used in cleavage reactions with plasmid DNA comprising a target sequence and a PAM. Stating with a TTTg PAM, the PAM was mutated to each of the sequences shown in FIG. 14C to assess the PAM requirement. The products of the cleavage reactions were analyzed by gel electrophoresis, as seen in FIG. 14C. FIG. 14D provides the quantification of the gels shown in FIG. 14C. Together, the data in FIG. 14C and FIG. 14D demonstrate a NTNN PAM for DNA cleavage by CasΦ.20, CasΦ.24 and CasΦ.25.

This example demonstrates PAM sequences that enable CasΦ polypeptides to be targeted to a target sequence.

### Example 20

### CasΦ-Mediated Genome Editing in HEK293T Cells

This example illustrates the ability of CasΦ polypeptides to mediate genome editing in HEK293T cells, a cell line which is widely used in biological research. In this study, a CasΦ.12 plasmid, including both CasΦ polypeptide sequence and gRNA sequence, sometimes called an all-in-one, was delivered via lipofection. Spacers targeted exon 4 of the Fut8 gene. The spacer sequences are provided in TABLE 7. Cells were transfected on day 0 and harvested for analysis on day 5. As shown in FIG. 15, the target locus was modified following delivery of CasΦ.12 and gRNA 2. Cas9 was delivered to HEK293T cells to provide a positive control and no modification was detected when a non-targeting (NT) gRNA was used. The presence of indels was confirmed by next generation sequence analysis. The sample targeted by CasΦ.12 and gRNA 2 is shown in FIG. 15. The next generation sequence analysis revealed a diverse pattern of indels. The most frequent mutations were deletion mutations of 4 to 18 base pairs. The frequency of mutations was quantified and is illustrated as “% modified”, which is defined as the % of modification in the DNA sequence when aligned to unedited cells. Modifications can be deletions, insertions and substitutions.

This example demonstrates the use of CasΦ.12 as a robust genome editing tool.

TABLE 7

| | | Spacer sequence (5′ → 3′) |
|---|---|---|
| Name | Target | [SEQ ID NO] |
| Fut8_1 | CasPhi target | GAAGAGCAGCTTGTTAAGGC |
| | | (SEQ ID NO: 1424) |
| Fut8_2 | CasPhi target | GCCTTAACAAGCTGCTCTTC |
| | | (SEQ ID NO: 1425) |
| Fut8_3 | Cas9 target | ATTGATCAGGGGCCAgctat |
| | (control) | (SEQ ID NO: 1426) |
| Fut8_4 | Cas9 target | Acgcgtactcttcctatagc |
| | (control) | (SEQ ID NO: 1427) |
| NT | Non target | CGTGATGGTCTCGATTGAGT |
| | | (SEQ ID NO: 1428) |

### Example 21

### CasΦ-Mediated Genome Editing in CHO Cells

This example illustrates the ability of CasΦ polypeptides to mediate genome editing in CHO cells, an epithelial cell line which is frequently used in biological and medical research. To test the function of CasΦ.12 in CHO cells, 40 pmol CasΦ.12 was complexed to its native crRNA (2.5:1 crRNA:CasΦ). To prepare a mastermix of CasΦ.12 RNP, 3 μl crRNA (at 100 nM) was added to 1.6 μl CasΦ.12 (at 75 μM). Spacer sequences are provided in Table 8. The RNP complexes were incubated at 37° C. for 30 minutes. CHO cells were resuspended at 1.2×10⁶ cells/ml in SF solution (Lonza). 40 μl of the cell suspension was added to the RNP complexes and 20 μl of the resultant suspension was then transferred to individual tubes for nucleofection. Lonza setting FF-137 was used to nucleofect the CHO cells. Cells were then harvested for analysis on day 5. As shown in FIG. 16A, CasΦ.12 induced the generation of indels in each of the endogenous genes tested (Bak1, Bax and Fut8). The ability of CasΦ.12 to induce indel mutations in each of these genes is further shown in FIG. 16F for Bak1, FIG. 16G for Bax and FIG. 16H for Fut8. Spacer sequences for FIG. 16F, FIG. 16G and FIG. 16H are provided in Tables F, G, and H, respectively. The data shown in FIG. 16F-H were produced with 200,000 CHO cells per transfection, RNP complexed with 250 pmol of CasΦ.12, and full-length unmodified guide RNA in molar excess relative to CasΦ.12, using the same Lonza reagents described for producing data presented in FIGS. 16A-E.

TABLE 8

| | | Repeat + Spacer sequence (5′ → 3′), |
|---|---|---|
| Name | Spacer sequence (5′ → 3′) | shown as DNA |
| Bak1_1 | GAAGCTATGTTTTCCAT | CTTTCAAGACTAATAGATTGCTCCTTACGA |
| | CTC (SEQ ID NO: 443) | GGAGACGAAGCTATGTTTTCCATCTC (SEQ |
| | | ID NO: 1197) |
| Bak1_2 | GCAGGGGCAGCCGCCC | CTTTCAAGACTAATAGATTGCTCCTTACGA |
| | CCTG | GGAGACGCAGGGGCAGCCGCCCCCTG |
| | (SEQ ID NO: 444) | (SEQ ID NO: 1198) |
| Bak1_3 | CTCCTAGAACCCAACA | CTTTCAAGACTAATAGATTGCTCCTTACGA |
| | GGTA | GGAGACCTCCTAGAACCCAACAGGTA |
| | (SEQ ID NO: 445) | (SEQ ID NO: 1199) |
| Bak1_4 | GAAAGACCTCCTCTGTG | CTTTCAAGACTAATAGATTGCTCCTTACGA |
| | TCC (SEQ ID NO: 446) | GGAGACGAAAGACCTCCTCTGTGTCC (SEQ |
| | | ID NO: 1200) |
| Bak1_5 | TCCATCTCGGGGTTGGC | CTTTCAAGACTAATAGATTGCTCCTTACGA |
| | AGG (SEQ ID NO: 447) | GGAGACTCCATCTCGGGGTTGGCAGG |
| | | (SEQ ID NO: 1201) |
| Bak1_6 | TTCCTGATGGTGGAGAT | CTTTCAAGACTAATAGATTGCTCCTTACGA |
| | GGA (SEQ ID NO: 448) | GGAGACTTCCTGATGGTGGAGATGGA |
| | | (SEQ ID NO: 1202) |
| Bax_1 | CTAATGTGGATACTAAC | CTTTCAAGACTAATAGATTGCTCCTTACGA |
| | TCC (SEQ ID NO: 479) | GGAGACCTAATGTGGATACTAACTCC (SEQ |
| | | ID NO: 1269) |
| Bax_2 | TTCCGTGTGGCAGCTGA | CTTTCAAGACTAATAGATTGCTCCTTACGA |
| | CAT (SEQ ID NO: 480) | GGAGACTTCCGTGTGGCAGCTGACAT (SEQ |
| | | ID NO: 1270) |
| Bax_3 | CTGATGGCAACTTCAAC | CTTTCAAGACTAATAGATTGCTCCTTACGA |
| | TGG (SEQ ID NO: 481) | GGAGACCTGATGGCAACTTCAACTGG |
| | | (SEQ ID NO: 1271) |
| Bax_4 | TACTTTGCTAGCAAACT | CTTTCAAGACTAATAGATTGCTCCTTACGA |
| | GGT (SEQ ID NO: 482) | GGAGACTACTTTGCTAGCAAACTGGT (SEQ |
| | | ID NO: 1272) |
| Bax_5 | AGCACCAGTTTGCTAGC | CTTTCAAGACTAATAGATTGCTCCTTACGA |
| | AAA (SEQ ID NO: 483) | GGAGACAGCACCAGTTTGCTAGCAAA |
| | | (SEQ ID NO: 1273) |
| Bax_6 | AACTGGGGCCGGGTTG | CTTTCAAGACTAATAGATTGCTCCTTACGA |
| | TTGC (SEQ ID NO: 484) | GGAGACAACTGGGGCCGGGTTGTTGC |
| | | (SEQ ID NO: 1274) |
| Fut8_1 | CCACTTTGTCAGTGCGT | CTTTCAAGACTAATAGATTGCTCCTTACGA |
| | CTG (SEQ ID NO: 507) | GGAGACCCACTTTGTCAGTGCGTCTG (SEQ |
| | | ID NO: 1325) |
| Fut8_2 | CTCAATGGGATGGAAG | CTTTCAAGACTAATAGATTGCTCCTTACGA |
| | GCTG (SEQ ID NO: 508) | GGAGACCTCAATGGGATGGAAGGCTG |
| | | (SEQ ID NO: 1326) |
| Fut8_3 | AGGAATACATGGTACA | CTTTCAAGACTAATAGATTGCTCCTTACGA |
| | CGTT (SEQ ID NO: 509) | GGAGACAGGAATACATGGTACACGTT |
| | | (SEQ ID NO: 1327) |
| Fut8_4 | AAGAACATTTTCAGCTT | CTTTCAAGACTAATAGATTGCTCCTTACGA |
| | CTC (SEQ ID NO: 510) | GGAGACAAGAACATTTTCAGCTTCTC (SEQ |
| | | ID NO: 1328) |
| Fut8_5 | ATCCACTTTCATTCTGC | CTTTCAAGACTAATAGATTGCTCCTTACGA |
| | GTT (SEQ ID NO: 511) | GGAGACATCCACTTTCATTCTGCGTT (SEQ |
| | | ID NO: 1329) |
| Fut8_6 | TTTGTTAAAGGAGGCA | CTTTCAAGACTAATAGATTGCTCCTTACGA |
| | AAGA (SEQ ID NO: 512) | GGAGACTTTGTTAAAGGAGGCAAAGA |
| | | (SEQ ID NO: 1330) |

The inventors went on to demonstrate the ability of CasΦ.12 to mediate gene editing via the homology directed repair pathway. The inventors tested DNA donor oligos with 25 bp, 50 bp or 90 bp homology arms (HA), as shown in FIG. 16B. The donor oligos were delivered to CHO cells with or without CasΦ.12 and crRNA. As seen in FIG. 16C, indels were not detected in the absence of CasΦ.12. Whereas, indels were detected in the presence of CasΦ.12 and confirmed by sequencing the endogenous targeted locus (FIG. 16D). The sequencing analysis also showed the successful incorporation of a DNA donor oligo into the endogenous targeted locus (FIG. 16E).

The inventors further demonstrated the ability of CasΦ.12 to mediate gene editing of Bax and Fut8 genes via the homology directed repair pathway. In this additional study, DNA donor oligos with 20 bp, 25 bp, 30 bp or 40 bp 90 bp HA were used, shown in FIG. 161. These DNA donor oligos were either unmodified or modified with phosphorothioate (PS) bonds between the first 5′, and the last two 3′ bases. As shown in FIG. 16J, CasΦ.12 mediated successful incorporation of a DNA donor oligo into the endogenous targeted locus. Finally, the inventors further optimized CasΦ.12-mediated genome editing of Fut8 using AAV6 delivery of the DNA donor. In this study, CHO cells were transfected with Fut8-targeting RNP (500 pmol) using Lonza nucleofection protocols. AAV6 donors at different MOIs were added to cells immediately after transfection. The frequency of indels and HDR was analyzed by NGS. As shown in FIG. 16K and FIG. 16L, CasΦ.12 induced the generation of indels and HDR.

These data further demonstrate the utility of CasΦ polypeptides as a genome editing tool.

### Example 22

### CasΦ-Mediated Genome Editing in K562 Cells

This example illustrates the ability of CasΦ polypeptides to mediate genome editing in K562 cells, a myelogenous leukemia cell line which is particularly useful for biological and medical research by virtue of its amenability for nucleofection by electroporation. In this study, K562 cells were nucleofected with Cas9 or CasΦ.12. To nucleofect the cells, 150,000 cells in SF solution (SF Cell Line 96 Amaxa) were added to the amount of plasmid (expressing the gRNA targeting the Fut8 gene and either Cas9 or CasΦ.12) indicated in FIG. 17. Amaxa program 96-FF-120 was used to nucleofect the cells. The cells were harvested two days after nucleofection and the frequency of indel mutations was determined. As shown in FIG. 17, as the amount of CasΦ.12 plasmid increased, the amount of indels detected in the endogenous Fut8 gene also increased.

### Example 23

### CasΦ-Mediated Genome Editing in Primary Cells

This example illustrates the ability of CasΦ polypeptides to mediate genome editing in primary cells, such as T cells. In this study, CasΦ.12 was delivered to human T cells. CasΦ.12 was complexed to its native crRNA comprising the spacer sequence 5′-GGGCCGAGAUGUCUCGCUCC-3′ (SEQ ID NO: 1429). Complexes were formed in a 3:1 ratio of crRNA:protein. For nucleofection, 50 pmol RNP was mixed with 320,000 cells per well and the Amaxa EH115 program was used. Immediately after nucleofection, 80 μl pre-warmed culture medium was added to each well. The cells were then left in the cuvette plate for 15 minutes before transfer to the culture plate. Genomic DNA was extracted from cells on day 3 and day 5. Flow cytometry analysis was performed on day 5. As shown in FIG. 18A, when CasΦ.12 was delivered with a gRNA targeting the endogenous beta-2 microglobulin (B2M) gene, a distinct population of B2M-negative cells was detected by flow cytometry analysis demonstrating the CasΦ.12-mediated knockout of the endogenous B2M gene. In the absence of the B2M-targeting gRNA, the population of B2M-negative cells was not observed by flow cytometry. Indels were confirmed by next generation sequencing analysis, as shown FIG. 18C, and quantified, as shown in FIG. 18B.

The inventors went on to use CasΦ.12 to target the T-cell receptor alpha-constant (TRAC) gene. Knockout of the TRAC gene prevents expression of the T cell receptor. Accordingly, TRAC knockout T cells are beneficial for T cell therapies (e.g. CAR-T cell therapies) because TRAC knockout T cells have a longer half-life in vivo as the T cells have less potential to attack the recipient's normal cells. In this study, CasΦ.12 and gRNA targeting the TRAC gene (CasPhi1 or CasPhi7) were delivered to T cells. As shown in FIG. 18D, the delivery of the CasΦ.12 and the gRNA resulted in a population of TRAC-negative cells, which were detected by flow cytometry. The inventors went on to confirm the presence of indel mutations by sequencing the target locus. As shown in FIG. 18E, the sequence analysis revealed insertion, deletion and substitution mutations at the endogenous targeted locus. The frequency of indel mutations was quantified, as shown in FIG. 18F.

These data demonstrate the utility of CasΦ polypeptides as a robust genome editing tool in primary human cells.

### Example 24

### Separable DNA Strand Cleavage Reactions of CasΦ Nucleases

This example further illustrates the mechanism of DNA strand cleavage by CasΦ polypeptides. In this study, CasΦ.4, CasΦ.12 and CasΦ.18 were complexed with their native crRNA. RNP complexes were formed by a 20 minute incubation at room temperature. The target plasmid was a 2.1 kb plasmid containing the target sequence TATTAAATACTCGTATTGCTGTTCGATTAT (SEQ ID NO: 10⁸). The cleavage reaction was carried out at 37° C. and had a duration of 30 minutes. The cleavage products were then analyzed by gel electrophoresis. As shown in FIG. 19, CasΦ polypeptides nick supercoiled (sc) DNA by cleaving the non-target DNA strand. Some CasΦ polypeptides, such as CasΦ.4 and CasΦ.12, then go on to cleave the second (target) strand to generate a linear product from a plasmid target. Whereas some CasΦ polypeptides, such as CasΦ.18, function as nickases and do not go on to cleave the second strand. CasΦ cleavage activity is dependent on metal cations, such as Mg²⁺. Varying the concentration of Mg²⁺ allows the cleavage of the first strand and then second strand by CasΦ.4 and CasΦ.12 to be visualized. As the concentration of Mg²⁺ increases, the amount of linearized product detected increases indicating that the second strand has been cleaved in the CasΦ.4 and CasΦ.12 reactions.

### Example 25

### Detection of a Target Nucleic Acid by CasΦ Polypeptides

This example illustrates the use of CasΦ.4 and CasΦ.18 in a nucleic acid detection assay by virtue of trans cleavage activity of ssDNA. In this study, 100 nM RNP was prepared and used in a detection assay. In the detection assay, the target dsDNA was at a concentration of 10 nM and the ssDNA reporter molecule was at a concentration of 100 nM. The target dsDNA included 5 target sequences, which were targeted by a pool of 5 gRNAs) with 7 base pairs flanking the 20 nucleotide target sequences on both 5′ and 3′ sides, as shown in FIG. 20. The detection assay was carried out at 37° C. The buffer conditions provided in TABLE 9 were tested in the detection assay. All buffers were supplemented with 0.1 mg/ml BSA and 1 mM TCEP. As seen in FIG. 20, when a gRNA (complexed to a CasΦ polypeptide) hybridizes to a target nucleic acid, the CasΦ 's trans cleavage activity is activated such that a labeled ssDNA reporter is degraded. The degradation of the ssDNA reporter is detected as fluorescence thus allowing CasΦ polypeptides to be used in assays to achieve fast and high-fidelity detection of target nucleic acid molecules in a sample. As shown in FIG. 20, high pH (e.g. 8-9) and high Mg²⁺ concentration (e.g. 12-15 mM) provided preferred conditions for the detection assay.

TABLE 9

| buffer ID # | pH | 1X NaCl (mM) | 1X MgCl₂ (mM) |
|---|---|---|---|
| 1 | 9 | 150 | 15 |
| 2 | 9 | 150 | 3 |
| 3 | 7.5 | 0 | 3 |
| 4 | 9 | 0 | 3 |
| 5 | 9 | 0 | 15 |
| 6 | 7.5 | 150 | 3 |
| 7 | 7.5 | 150 | 15 |
| 8 | 8 | 37.5 | 3 |
| 9 | 8.5 | 150 | 12 |
| 10 | 7.5 | 0 | 15 |
| 11 | 8.5 | 0 | 6 |
| 12 | 9 | 150 | 3 |
| 13 | 9 | 0 | 3 |
| 14 | 9 | 150 | 15 |
| 15 | 8 | 150 | 6 |
| 16 | 7.5 | 150 | 15 |
| 17 | 8 | 112.5 | 15 |
| 18 | 9 | 0 | 15 |
| 19 | 7.5 | 150 | 3 |
| 20 | 8.5 | 112.5 | 3 |
| 21 | 8.5 | 37.5 | 12 |
| 22 | 7.5 | 0 | 3 |
| 23 | 8.5 | 112.5 | 6 |
| 24 | 7.5 | 37.5 | 6 |
| 25 | 8 | 0 | 12 |
| 26 | 7.5 | 112.5 | 6 |
| 27 | 8.5 | 37.5 | 15 |
| 28 | 9 | 37.5 | 6 |
| 29 | 9 | 112.5 | 12 |
| 30 | 7.5 | 37.5 | 12 |
| 31 | 7.5 | 0 | 15 |
| 32 | 7.5 | 112.5 | 12 |

These data demonstrate the utility of CasΦ polypeptides in nucleic acid detection assays.

### Example 26

### High Efficiency of CasΦ Polypeptide-Mediated Genome Editing in Primary Cells

The present example shows that CasΦ.12 mediates high genome editing efficiency that is comparable the editing efficiency mediated by Cas9. Results of the study are shown in FIG. 21. In this study, CasΦ.12 mRNA (SEQ TD NO: 107) with a gRNA (CUUUCAAGACUAAUAGAUUGCUCCUUACGAGGAGACGGGCCGAGAUGUCUCGCUCC (SEQ ID NO: 1430)); spacer sequence is bold and underlined) or Cas9 mRNA with a gRNA (GGCCGAGATGTCTCGCTCCG (SEQ TD NO: 1431)) was delivered to T cells. gRNAs used in this study targeted the B32M gene. For nucleofection, T cells were resuspended in BTXpress electroporation medium (5×10⁵ cells per well) and mixed with CasΦ.12 or Cas9 mRNA and 500 pmol gRNA. Cells were collected on day 2 for extraction of genomic DNA, and the frequency of indel mutations was determined. As shown in FIG. 21A, when 20 μg of CasΦ.12 mRNA was delivered with gRNA to T cells, high genome editing efficiency was achieved, and this was at a similar level to of genome editing achieved using Cas9. Cells were also collected on Day 2 for flow cytometry to determine the frequency of B12M knockout. As shown in FIG. 21B and quantified in FIG. 21A, a similar percentage of B12M-negative cells were detected after delivery of CasΦ.12 or Cas9 mRNA. Accordingly, this example demonstrates high efficiency of CasΦ polypeptide-mediated genome efficiency in primary cells.

### Example 27

### CasΦ Polypeptide-Mediated Genome Editing in CHO Cells

This present example describes the identification of optimized gRNAs for CasΦ.12-mediated genome editing in CHO cells. In this study, CasΦ.12 polypeptides (SEQ ID NO: 107) were complexed with a gRNA shown in TABLE 10. CHO cells were resuspended in SF solution and Lonza setting FF-137 was used to nucleofect the cells (200,000 cells per well) with 250 pmol RNP. Genomic DNA was extracted and the presence of indels was confirmed by next generation sequence analysis. FIG. 22A shows the frequency of indel mutations induced by CasΦ.12 polypeptides complexed with a 2′fluoro modified gRNA. As shown in FIG. 22B, gRNAs with ˜20% or greater editing efficiency were identified.

TABLE 10

| | | RNA sequence (5′ → 3′), shown as |
|---|---|---|
| Name | Spacer sequence (5′ → 3′) | DNA |
| R2849 Bak1_nsd_ | CTGACTCCCAGCTCTGA | CTTTCAAGACTAATAGATTGCTCC |
| sg1 | CCC (SEQ ID NO: 449) | TTACGAGGAGACCTGACTCCCAG |
| | | CTCTGACCC (SEQ ID NO: 1203) |
| R2855 Bak1_nsd_ | CCATCTCCACCATCAGG | CTTTCAAGACTAATAGATTGCTCC |
| sg7 | AAC (SEQ ID NO: 455) | TTACGAGGAGACCCATCTCCACC |
| | | ATCAGGAAC (SEQ ID NO: 1209) |
| R3977 | TCCAGACGCCATCTTTCA | CTTTCAAGACTAATAGATTGCTCC |
| Bak1_exon1_sg1 | GG | TTACGAGGAGACTCCAGACGCCA |
| | (SEQ ID NO: 465) | TCTTTCAGG (SEQ ID NO: 1219) |
| R3978 | TGGTAAGAGTCCTCCTG | CTTTCAAGACTAATAGATTGCTCC |
| Bak1_exon1_sg2 | CCC | TTACGAGGAGACTGGTAAGAGTC |
| | (SEQ ID NO: 466) | CTCCTGCCC (SEQ ID NO: 1220) |
| R3979 | TTACAGCATCTTGGGTC | CTTTCAAGACTAATAGATTGCTCC |
| Bak1_exon3_sg1 | AGG | TTACGAGGAGACTTACAGCATCT |
| | (SEQ ID NO: 467) | TGGGTCAGG (SEQ ID NO: 1221) |
| R3980 | GGTCAGGTGGGCCGGCA | CTTTCAAGACTAATAGATTGCTCC |
| Bak1_exon3_sg2 | GCT | TTACGAGGAGACGGTCAGGTGGG |
| | (SEQ ID NO: 468) | CCGGCAGCT (SEQ ID NO: 1222) |
| R3981 | CTATCATTGGAGATGAC | CTTTCAAGACTAATAGATTGCTCC |
| Bak1_exon3_sg3 | ATT | TTACGAGGAGACCTATCATTGGA |
| | (SEQ ID NO: 469) | GATGACATT (SEQ ID NO: 1223) |
| R3982 | GAGATGACATTAACCGG | CTTTCAAGACTAATAGATTGCTCC |
| Bak1_exon3_sg4 | AGA | TTACGAGGAGACGAGATGACATT |
| | (SEQ ID NO: 470) | AACCGGAGA (SEQ ID NO: 1224) |
| R3983 | TGGAACTCTGTGTCGTAT | CTTTCAAGACTAATAGATTGCTCC |
| Bak1_exon3_sg5 | CT | TTACGAGGAGACTGGAACTCTGT |
| | (SEQ ID NO: 471) | GTCGTATCT (SEQ ID NO: 1225) |
| R3984 | CAGAATTTACTGGAGCA | CTTTCAAGACTAATAGATTGCTCC |
| Bak1_exon3_sg6 | GCT | TTACGAGGAGACCAGAATTTACT |
| | (SEQ ID NO: 472) | GGAGCAGCT (SEQ ID NO: 1226) |
| R3985 | ACTGGAGCAGCTGCAGC | CTTTCAAGACTAATAGATTGCTCC |
| Bak1_exon3_sg7 | CCA | TTACGAGGAGACACTGGAGCAGC |
| | (SEQ ID NO: 473) | TGCAGCCCA (SEQ ID NO: 1227) |
| R3986 | CCAGCTGTGGGCTGCAG | CTTTCAAGACTAATAGATTGCTCC |
| Bak1_exon3_sg8 | CTG | TTACGAGGAGACCCAGCTGTGGG |
| | (SEQ ID NO: 474) | CTGCAGCTG (SEQ ID NO: 1228) |
| R3987 | GTAGGCATTCCCAGCTG | CTTTCAAGACTAATAGATTGCTCC |
| Bak1_exon3_sg9 | TGG | TTACGAGGAGACGTAGGCATTCC |
| | (SEQ ID NO: 475) | CAGCTGTGG (SEQ ID NO: 1229) |
| R3988 | GTGAAGAGTTCGTAGGC | CTTTCAAGACTAATAGATTGCTCC |
| Bak1_exon3_sg10 | ATT | TTACGAGGAGACGTGAAGAGTTC |
| | (SEQ ID NO: 476) | GTAGGCATT (SEQ ID NO: 1230) |
| R3989 | ACCAAGATTGCCTCCAG | CTTTCAAGACTAATAGATTGCTCC |
| Bak1_exon3_sg11 | GTA | TTACGAGGAGACACCAAGATTGC |
| | (SEQ ID NO: 477) | CTCCAGGTA (SEQ ID NO: 1231) |
| R3990 | CCTCCAGGTACCCACCA | CTTTCAAGACTAATAGATTGCTCC |
| Bak1_exon3_sg12 | CCA | TTACGAGGAGACCCTCCAGGTAC |
| | (SEQ ID NO: 478) | CCACCACCA (SEQ ID NO: 1232) |

### Example 28

### Minimal Off-Target Effects of CasΦ Polypeptides

This example illustrates the off-target profiles of CasΦ.12 and Cas9. A major challenge in the translation of CRISPR/Cas9 technology into the clinic has been overcoming off-target effects. Off-target effects arise where a gRNA tolerates mismatches in complementarity of the gRNA and target sequence, and so the gRNA hybridizes to a sequence that is not the target sequence. Off-target effects are a source of major concern as it is important to avoid the production in unnecessary mutations that could be detrimental. In this study, CIRCLE-seq was performed to detect off-target sites (Tsai et al. 2017 Nature Methods). Sequencing was performed on genomic DNA extracted from CHO cells that had been transfected with CasΦ.12 polypeptide (SEQ ID NO: 107) and a gRNA targeting Fut8, CasΦ.12 polypeptide and a gRNA targeting BAX or Cas9 polypeptide and a gRNA targeting BAX. As shown in FIG. 23A, CasΦ.12 targeting Fut8 induced minimal off-target mutations. FIG. 23D shows the off-target mutations induced by Cas9 editing of Fut8. Similarly, CasΦ.12 targeting BAX induced minimal off-target mutations, as shown in FIG. 23B. Cas9 targeting BAX induced a higher percentage of off-targets mutations, as shown in FIG. 23C, compared to CasΦ.12. Cas9 targeting Bak1 also induced a higher percentage of off-targets mutations, as shown in FIG. 23E, compared to CasΦ.12, as shown in FIG. 23F.

In a further study, GUIDE-Seq was performed to detect off-target sites (Tsai et al. 2015 Nature Biotechnology). Sequencing was performed on genomic DNA extracted from HEK293 cells following delivery of either CasΦ.12 polypeptide or Cas9 polypeptide and a gRNA targeting human Fut8. As shown in FIG. 23G, no off target mutations were detected in the CasΦ.12 polypeptide sample. Whereas, several off-target mutations were detected in Cas9 polypeptide sample, as shown in FIG. 23H. Accordingly, this example demonstrates that CasΦ polypeptides have fewer off-target effects than Cas9.

### Example 29

### CasΦ Polypeptide-Mediated Genome Editing Via Homology Directed Repair (HDR)

The present example illustrates the ability of that CasΦ.12 to mediate HDR. In this study, CasΦ.12 polypeptide (SEQ ID NO: 107) was complexed with a gRNA (CUUUCAAGACUAAUAGAUUGCUCCUUACGAGGAGACGAGUCUCUCAGCUGGUAC AC (SEQ ID NO: 1432)) targeting the TRAC gene and delivered to T cells. RNP complexes were formed by a 10 minute incubation at room temperature. T cells were resuspended at 5×10⁵ cells/20 μL in electroporation solution (Lonza). T cells were nucleofected using the Amaxa P3 kit and Amaxa 4D Nucleofector with pulse code EH115. Immediately after nucleofection, 80 μl pre-warmed culture medium was added to each well. The cells were then left in the cuvette plate for 10 minutes before transfer to the culture plate. Cells were harvested and genomic DNA was extracted. The frequency of indel mutations HDR was determined and shown in FIG. 24A. The frequency of indel mutations and HDR was combined to determine the frequency of modification. Flow cytometry was also performed to determine the frequency of TRAC knockout, as assessed by the loss of CD3 at the cell surface. FIG. 24A shows CasΦ.12-mediated gene editing via the HDR pathway. FIG. 24B shows a schematic of the donor oligonucleotide. Thus, this example demonstrates the use of CasΦ polypeptides as robust genome editing tools.

### Example 30

### Multiplex Genome Editing with CasΦ Polypeptides

This example illustrates the ability of CasΦ RNP complexes to target multiple genes simultaneously. In this study, gRNAs targeting B2M or TRAC were incubated with CasΦ.12 polypeptides (SEQ ID NO: 107) for 10 minutes at room temperature to form RNP complexes. RNP complexes were formed with a variety of gRNAs with different modifications (unmodified, 2′-O-methyl on the last 3′ nucleotide of the crRNA (1me), 2′-O-methyl on the last two 3′ nucleotides of the crRNA (2me) and 2′-O-methyl on the last three 3′ nucleotides of the crRNA(3me)) and with different repeat and spacer sequences (20-20, which corresponds to 20 nucleotide repeat and 20 nucleotide spacer, and 20-17, which corresponds to 20 nucleotide repeat and 17 nucleotide spacer), as shown in TABLE 11. B2M targeting RNPs, TRAC targeting RNPs or B2M targeting RNPs and TRAC targeting RNPs were added to T cells. T cells were resuspended at 5×10⁵ cells/20 μL in Nucleofection P3 solution and an Amaxa 4D 96-well electroporation system with pulse code EH115 was used to nucleofect the cells. Immediately after nucleofection, 85 μl pre-warmed culture medium was added to each well. The cells were then left in the cuvette plate for 10 minutes before transfer to the culture plate. On Day 3, genomic DNA was extracted. On Day 5, cells were harvested for flow cytometry. Quantification of the percentage of B2M-negative and CD3-negative cells is shown in FIG. 25A for gRNAs with a repeat length of 20 nucleotides and a spacer length of 20 nucleotides, and in FIG. 25B for gRNAs with a repeat length of 20 nucleotides and a spacer length of 17 nucleotides. Corresponding flow cytometry panels can be seen in FIG. 25C for gRNAs of different repeat and spacer lengths and with different modifications.

In a further study, RNP complexes were formed using CasΦ.12 and modified gRNAs (unmodified, line, 2me, 3me, 2′-fluoro on the last 3′ nucleotide of the crRNA (1F), 2′-fluoro on the last two 3′ nucleotides of the crRNA (2F) and 2′-fluoro on the last three 3′ nucleotides of the crRNA (3F)) with different lengths of spacer sequences (20-20 and 20-17 as above) that target TRAC. T cells were nucleofected with RNP complexes (125 pmol) using the P3 primary cell nucleofection kit and an Amaxa 4D 96-well electroporation system with pulse code EHQ115. As shown in FIG. 25D, ˜90% editing efficiency was achieved using CasΦ.12 and modified gRNAs. FIG. 25E shows a flow cytometry plot illustrating ˜90% TRAC knockout in T cells after delivery of CasΦ.12 and modified gRNAs. This data further demonstrates the ability of CasΦ to mediate high efficiency genome editing.

TABLE 11

| | | | Repeat | Spacer | |
|---|---|---|---|---|---|
| | | | sequence | sequence | crRNA sequence |
| Name | Target | Modification | (5′ → 3′) | (5′ → 3′) | (5′ → 3′) |
| R3150 | B2M | Unmodified, | AUUGCUC | CAGUGGGGG | AUUGCUCCUUAC |
| 20-20 | Exon 2 | 2′OMe at last | CUUACGA | UGAAUUCAG | GAGGAGACCAG |
| | | 3′ base (1me) | GGAGAC | UG (SEQ ID | UGGGGGUGAAU |
| | | 2′OMe at last | (SEQ ID NO: | NO: 1434) | UCAGUG (SEQ ID |
| | | two 3′ bases | 1433) | | NO: 1435) |
| | | (2me) | | | |
| | | 2′OMe at last | | | |
| | | three 3′ bases | | | |
| | | (3me) | | | |
| R3042 | TRAC | Unmodified, | AUUGCUC | GAGUCUCUC | AUUGCUCCUUAC |
| 20-20 | Exon 1 | 1me | CUUACGA | AGCUGGUAC | GAGGAGACGAG |
| | | 2me | GGAGAC | AC (SEQ ID | UCUCUCAGCUGG |
| | | 3me | (SEQ ID NO: | NO: 1436) | UACAC (SEQ ID |
| | | | 1433) | | NO: 1437) |
| R3150 | B2M | Unmodified, | AUUGCUC | CAGUGGGGG | AUUGCUCCUUAC |
| 20-17 | Exon 2 | 1me | CUUACGA | UGAAUUCA | GAGGAGACCAG |
| | | 2me | GGAGAC | (SEQ ID NO: | UGGGGGUGAAU |
| | | 3me | (SEQ ID NO: | 1438) | UCA (SEQ ID NO: |
| | | | 1433) | | 1439) |
| R3042 | TRAC | Unmodified, | AUUGCUC | CAGUGGGGG | AUUGCUCCUUAC |
| 20-17 | Exon 1 | 1me | CUUACGA | UGAAUUCA | GAGGAGACGAG |
| | | 2me | GGAGAC | (SEQ ID NO: | UCUCUCAGCUGG |
| | | 3me | (SEQ ID NO: | 1440) | UA (SEQ ID NO: |
| | | | 1433) | | 1441) |

### Example 31

### CasΦ Polypeptides have an Extended Seed Region

The present example shows that CasΦ.12 has an extended seed region compared to Cas9 and does not tolerate mismatches in the complementarity of the spacer and target sequences within the first 1-16 nucleotides from the 5′ of the spacer sequence. In this study, CasΦ.12 (SEQ ID NO: 107) was complexed with a gRNA targeting TRAC gene and delivered to T cells. Spacer sequences contained a single mismatch at the position indicated in FIG. 26A or a mismatch at each of the two positions indicated in FIG. 26B. Mismatches were generated by substituting a purine for a purine (i.e. A to G and vice versa) and a pyrimidine for a pyrimidine (i.e. U to C and vice versa). RNP complexes were formed by a 10 minute incubation at room temperature. T cells were resuspended at 5×10⁵ cells/20 μL in electroporation solution (Lonza). Amaxa P3 kit and Amaxa 4D Nucleofector was used to nucleofect the T cells. Immediately after nucleofection, 80 μl pre-warmed culture medium was added to each well. The cells were then left in the cuvette plate for 10 minutes before transfer to the culture plate. Cells were harvested for extraction of genomic DNA to determine the frequency of indel mutations and for flow cytometry to determine the percentage of CD3 knockout cells. As shown in FIG. 26A, no indel mutations or CD3 knockout were detected when there was a single mismatch in the complementarity of the spacer and target sequences at positions 1-16 from the 5′ end of the spacer sequence. Similarly, no indels or CD3 knockout cells were detected when there was a double mismatch in the complementarity of the spacer and target sequences at positions 1-16 from the 5′ end of the spacer sequence as shown in FIG. 26B. The data shown in FIG. 26A and FIG. 26B demonstrate that CasΦ polypeptides do not tolerate mismatches in complementarity between the spacer sequence and target sequence in the 5′ 16 positions of the spacer. This region in which mismatches are not tolerated is known as the “seed region”. Thus the seed region of CasΦ.12 is the first 16 bases from the 5′ end of the spacer. In contrast, the seed region of Cas9 is much shorter and is reported to be only 5 nucleotides long (Wu et al., Quant Biol. 2014 June; 2(2): 59-70). Shorter seed regions result in increased likelihood of off-target effects because the likelihood of mismatches between the spacer and target occurring outside the seed region is increased. Accordingly, longer seed regions result in a reduced likelihood of off-target effects. The long seed region of CasΦ.12 is therefore advantageous over the short seed region of Cas9 and contributes to the reduced off-target effects of CasΦ.12. FIG. 26C and FIG. 26D provide schematics of the gRNAs with mismatches.

### Example 32

### Use of Modified Guide RNAs with CasΦ Polypeptides

This example illustrates the ability of CasΦ.12 to mediate genome editing in CHO cells with modified gRNAs. In this study, RNP complexes were formed using CasΦ.12 polypeptide (SEQ ID NO: 107) and a modified gRNA shown in TABLE 12. For nucleofection, 200 pmol RNP was mixed with 200,000 cells per well. CHO cells were resuspended in SF solution and Lonza setting FF-137 was used to nucleofect the cells. Genomic DNA was extracted 48 hours after transfection and the frequency of indel mutations was determined. As shown in FIG. 27A, several modified gRNAs with editing efficiency of ˜10% were identified. In a further study, additional modified gRNAs were tested. As shown in FIG. 27B, modified gRNAs with editing efficiency of up to 40-50% were identified.

gRNAs with phosphorothioate (PS) backbone modifications, 2′-fluoro (2′-F) and 2′-Methyl (2′OMe) sugar modifications are known to increase metabolic stability and binding affinity to RNA, and replacing RNA nucleotides with DNA generates gRNAs with highly efficient gene-editing activity compared to the natural crRNA (Rahdar et al, 2015, PNA; McMahon et al. 2017, Molecular Therapy Vol. 26 No 5).

TABLE 12

| SEQ | Name | | | | Name |
|---|---|---|---|---|---|
| ID | (FIG. | | | Full modified guide (repeat | (FIG. |
| NO. | 27A) | Modification | Position | and spacer) | 27A, B) |
| 1442 | R2466_ | 2′-O-Methyl | 2′OMe at 3 first | mC*mU*mU*UCAAGACUA | Synthe |
| | Mo | (2′OMe), 3′ | (5′) and last (3′) | AUAGAUUGCUCCUUACG | go_Mod |
| | 1 | phosphorothioate | bases, 3′ PS | AGGAGACAGGAAUACAU | |
| | | (PS) | bonds between | GGUACACmG*mU*mU* | |
| | | bonds | first 3 (5′) and | | |
| | | | last 2 (3′) bases | | |
| 1443 | R2466_ | 2′OMe, 3′, | 2′OMe at 3 first | mA*mA*mU*AGAUUGCUC | |
| | Mo | 25 nucleotide | (5′) and last (3′) | CUUACGAGGAGACAGGA | |
| | 2 | repeat | bases, 3′ PS | AUACAUGGUACACmG*m | |
| | | | bonds between | U*mU | |
| | | | first 3 (5′) and | | |
| | | | last 2 (3′) bases | | |
| 1444 | R2466_ | 2′-O- | 2′-O-Methoxy- | /52MOErA*/i2MOErA*/UA | |
| | Mo | methoxy- | ethyl bases at 2 | GAUUGCUCCUUACGAGG | |
| | 3 | ethyl bases | first (5′) and last | AGACAGGAAUACAUGGU | |
| | | | (3′) bases, 3′ PS | ACACG/i2MOErT/32MOErT | |
| | | | bonds between | | |
| | | | first 2 (5′) and | | |
| | | | last 2 (3′) bases | | |
| 1445 | R2466_ | 2′-Fluoro (2′- | First (5′) and last | /52FC/UUUCAAGACUAAU | |
| | Mo | F) | (3′) base | AGAUUGCUCCUUACGAG | |
| | 4 | | | GAGACAGGAAUACAUGG | |
| | | | | UACACGU/32FU/ | |
| 1446 | R2466_ | 2′-F, 25 | First (5′) and last | /52FA/AUAGAUUGCUCCU | 1F, 45F |
| | Mo | nucleotide | (3′) base | UACGAGGAGACAGGAAU | (25nt |
| | 5 | repeat | | ACAUGGUACACGU/32FU/ | R) |
| 1447 | R2466_ | 2′-F, PS, | First (5′) base | mC*U*UUCAAGACUAAUA | 1, 2 |
| | Mo | 2′OMe | 2′OMe, PS | GAUUGCUCCUUACGAGG | OMe- |
| | 6 | | between first | AGACAGGAAUACAUGGU | PS, 54, |
| | | | two(5′) bases, last | ACA/i2FC/i2FG/i2FU/32FU/ | 55, 56 |
| | | | 4 (3′) bases 2′-F | | ′F |
| 1448 | R2466_ | 2′-F, PS, | First (5′) base | mA*A*UAGAUUGCUCCUU | 1, 2 |
| | Mo | 2′OMe, 25 | 2′OMe, PS | ACGAGGAGACAGGAAUA | OMe- |
| | 7 | nucleotide | between first | CAUGGUACA/i2FC/i2FG/i2F | PS, 54, |
| | | repeat | two(5′) bases, last | U/32FU | 55, 56 |
| | | | 4 (3′)bases 2′-F | | ′F (25nt |
| | | | | | R) |
| 1449 | R2466_ | 2′-F | Last 4 (3′) bases | CUUUCAAGACUAAUAGA | 54, 55, |
| | Mo | | 2′-F | UUGCUCCUUACGAGGAG | 56 2′F |
| | 8 | | | ACAGGAAUACAUGGUAC | |
| | | | | A/i2FC/i2FG/i2FU/32FU | |
| 1450 | R2466_ | 2′-F, 25 | Last 4 (3′) bases | AAUAGAUUGCUCCUUAC | 54, 55, |
| | Mo | nucleotide | 2′-F | GAGGAGACAGGAAUACA | 56 2′F |
| | 9 | repeat | | UGGUACA/i2FC/i2FG/i2FU/ | (25 nt |
| | | | | 32FU | R) |
| 1451 | R2466_ | C3 Spacer, | First (5′) and last | CUUUCAAGACUAAUAGA | |
| | Mo | 21 nucleotide | (3′) base | UUGCUCCUUACGAGGAG | |
| | 10 | spacer | | ACAGGAAUACAUGGUAC | |
| | | | | ACGUUG | |
| 1452 | R2466_ | C3 Spacer, | First (5′) and last | AAUAGAUUGCUCCUUAC | |
| | Mo | 21 nucleotide | (3′) base | GAGGAGACAGGAAUACA | |
| | 11 | spacer, 25 | | UGGUACACGUUG | |
| | | nucleotide | | | |
| | | spacer | | | |
| 1453 | R2466_ | DNA bases + | 2′OMe at 3 | mC*mU*mU*UCAAGACUA | 1,2, 3 |
| | Mo | 2′OMe, PS | first(5′) bases, | AUAGAUUGCUCCUUACG | Ome- |
| | 12 | | last 4(3′) bases | AGGAGACAGGAAUACAU | PS 54, |
| | | | DNA | GGUACACGTT | 55, 56 |
| | | | | | DNA |
| 1454 | R2466_ | DNA | Last (3′) 4 | CUUUCAAGACUAAUAGA | |
| | Mo | nucleoside | nucleoside | UUGCUCCUUACGAGGAG | |
| | 13 | | | ACAGGAAUACAUGGUAC | |
| | | | | ACGTT | |
| 1455 | R2466_ | DNA | Nucleoside 1 of | CUUUCAAGACUAAUAGA | 1, 54, |
| | Mo | nucleosides | spacer and last | UUGCUCCUUACGAGGAG | 55, 56 |
| | 14 | | (3′) 4 nucleosides | ACAGGAAUACAUGGUAC | DNA |
| | | | | ACGTT | |
| 1456 | R2466_ | DNA | Nucleoside 8 of | CUUUCAAGACUAAUAGA | |
| | Mo | nucleosides | spacer and last | UUGCUCCUUACGAGGAG | |
| | 15 | | (3′) 4 nucleosides | ACAGGAAUACAUGGUAC | |
| | | | | ACGTT | |
| 1457 | R2466_ | DNA | Nucleoside 9 of | CUUUCAAGACUAAUAGA | |
| | Mo | nucleosides | spacer and last | UUGCUCCUUACGAGGAG | |
| | 16 | | (3′) 4 nucleosides | ACAGGAAUACAUGGUAC | |
| | | | | ACGTT | |
| 1458 | R2466_ | DNA | Nucleoside 1 and | CUUUCAAGACUAAUAGA | 1, 8, 54, |
| | Mo | nucleosides | 8 of spacer and | UUGCUCCUUACGAGGAG | 55, 56 |
| | 17 | | last (3′) 4 | ACAGGAAUACAUGGUAC | DNA |
| | | | nucleosides | ACGTT | |
| 1459 | R2466_ | DNA | Nucleoside 1 and | CUUUCAAGACUAAUAGA | |
| | Mo | nucleosides | 9 of spacer and | UUGCUCCUUACGAGGAG | |
| | 18 | | last (3′) 4 | ACAGGAAUACAUGGUAC | |
| | | | nucleosides | ACGTT | |
| 1460 | R2466_ | DNA | Nucleoside 1, 8 | CUUUCAAGACUAAUAGA | 1, 8, 9, |
| | Mo | nucleosides | and 9 of spacer | UUGCUCCUUACGAGGAG | 54, 55, |
| | 19 | | and last (3′) 4 | ACAGGAAUACAUGGUAC | 56 |
| | | | nucleosides | ACGTT | DNA |
| 1461 | R2466_ | DNA bases, | Nucleoside 1, 8 | AAUAGAUUGCUCCUUAC | |
| | Mo | 25 nucleotide | and 9 of spacer | GAGGAGACAGGAAUACA | |
| | 20 | repeat | and last (3′) 4 | UGGUACACGTT | |
| | | | nucleosides | | |
| 1462 | R2466_ | Poly-A-tail, | | AAUAGAUUGCUCCUUAC | |
| | Mo | repeat | | GAGGAGACAGGAAUACA | |
| | 21 | 25 nucleotide | | UGGUACACGUUAAAAAA | |
| | | | | A | |
| 1463 | R2466_ | DNA bases, | 2′OMe and PS at | mC*mU*mU*UCAAGACUA | 1, 2, 3 |
| | Mo | 2′OMe, PS | first 3(5′) bases, | AUAGAUUGCUCCUUACG | OMe, |
| | 22 | | DNA bases at 1,8 | AGGAGACAGGAAUACAU | 1, 8, 9, |
| | | | and 9 of spacer, | GGUACACGTT | 54, 55, |
| | | | PS at last 4 (3′) | | 56 |
| | | | bases | | DNA |
| 1464 | R2466_ | Unmodified, | | AAUAGAUUGCUCCUUAC | |
| | Mo | 25 nucleotide | | GAGGAGACAGGAAUACA | |
| | 23 | repeat | | UGGUACACGUU | |
| 1465 | R2466 | Unmodified | Unmodified | CUUUCAAGACUAAUAGA | |
| | (Unmodified) | | | UUGCUCCUUACGAGGAG | |
| | | | | ACAGGAAUACAUGGUAC | |
| | | | | ACGUU | |

### Example 33

### Optimization of Guide RNA Repeat and Spacer Length in CHO Cells

This example describes the optimization of repeat and spacer lengths of gRNAs for genome editing in CHO cells. In this study, RNP complexes were formed by incubating CasΦ.12 polypeptides (SEQ TD NO: 107) with a gRNA targeting Fut8 gene shown in TABLE 13. The gRNAs had different repeat lengths (20 to 36 nucleotides) or spacer lengths (15 to 30 nucleotides). Genomic DNA was extracted and the frequency of indel mutations was determined. For nucleofection, 250 pmol RNP was mixed with 200,000 cells per well. After 2 days, cells were collected and genomic DNA was extracted to determine the frequency of indel mutations. FIG. 28A shows the generation of indels by CasΦ.12 with gRNAs containing repeat sequences of different lengths. FIG. 28B the shows the generation of indels by CasΦ.12 with gRNAs containing spacer sequences of different lengths. The optimal gRNA for CasΦ.12-mediated genome editing in CHO cells was found to have a 20-nucleotide repeat length and a 17-nucleotide spacer length.

TABLE 13

| | | | Repeat | | |
|---|---|---|---|---|---|
| | Repeat | Spacer | sequence | Spacer sequence | crRNA sequence |
| Name | length | length | (5′ → 3′) | (5′ → 3′) | (5′ → 3′) |
| R3582 | 36 | 30 | CUUUCAAGA | AGGAAUACAU | CUUUCAAGACUAA |
| | | | CUAAUAGAU | GGUACACGUU | UAGAUUGCUCCUU |
| | | | UGCUCCUUA | GAAGAACAUU | ACGAGGAGACAGG |
| | | | CGAGGAGAC | (SEQ ID NO: | AAUACAUGGUACA |
| | | | (SEQ ID NO: | 1482) | CGUUGAAGAACAU |
| | | | 54) | | U (SEQ ID NO: 1499) |
| R3583 | 36 | 29 | CUUUCAAGA | AGGAAUACAU | CUUUCAAGACUAA |
| | | | CUAAUAGAU | GGUACACGUU | UAGAUUGCUCCUU |
| | | | UGCUCCUUA | GAAGAACAU | ACGAGGAGACAGG |
| | | | CGAGGAGAC | (SEQ ID NO: | AAUACAUGGUACA |
| | | | (SEQ ID NO: | 1483) | CGUUGAAGAACAU |
| | | | 54) | | (SEQ ID NO: 1500) |
| R3584 | 36 | 28 | CUUUCAAGA | AGGAAUACAU | CUUUCAAGACUAA |
| | | | CUAAUAGAU | GGUACACGUU | UAGAUUGCUCCUU |
| | | | UGCUCCUUA | GAAGAACA | ACGAGGAGACAGG |
| | | | CGAGGAGAC | (SEQ ID NO: | AAUACAUGGUACA |
| | | | (SEQ ID NO: | 1484) | CGUUGAAGAACA |
| | | | 54) | | (SEQ ID NO: 1501) |
| R3585 | 36 | 27 | CUUUCAAGA | AGGAAUACAU | CUUUCAAGACUAA |
| | | | CUAAUAGAU | GGUACACGUU | UAGAUUGCUCCUU |
| | | | UGCUCCUUA | GAAGAAC | ACGAGGAGACAGG |
| | | | CGAGGAGAC | (SEQ ID NO: | AAUACAUGGUACA |
| | | | (SEQ ID NO: | 1485) | CGUUGAAGAAC |
| | | | 54) | | (SEQ ID NO: 1502) |
| R3586 | 36 | 26 | CUUUCAAGA | AGGAAUACAU | CUUUCAAGACUAA |
| | | | CUAAUAGAU | GGUACACGUU | UAGAUUGCUCCUU |
| | | | UGCUCCUUA | GAAGAA (SEQ | ACGAGGAGACAGG |
| | | | CGAGGAGAC | ID NO: 1486) | AAUACAUGGUACA |
| | | | (SEQ ID NO: | | CGUUGAAGAA (SEQ |
| | | | 54) | | ID NO: 1503) |
| R3587 | 36 | 25 | CUUUCAAGA | AGGAAUACAU | CUUUCAAGACUAA |
| | | | CUAAUAGAU | GGUACACGUU | UAGAUUGCUCCUU |
| | | | UGCUCCUUA | GAAGA (SEQ | ACGAGGAGACAGG |
| | | | CGAGGAGAC | ID NO: 1487) | AAUACAUGGUACA |
| | | | (SEQ ID NO: | | CGUUGAAGA (SEQ |
| | | | 54) | | ID NO: 1504) |
| R3588 | 36 | 24 | CUUUCAAGA | AGGAAUACAU | CUUUCAAGACUAA |
| | | | CUAAUAGAU | GGUACACGUU | UAGAUUGCUCCUU |
| | | | UGCUCCUUA | GAAG (SEQ ID | ACGAGGAGACAGG |
| | | | CGAGGAGAC | NO: 1488) | AAUACAUGGUACA |
| | | | (SEQ ID NO: | | CGUUGAAG (SEQ ID |
| | | | 54) | | NO: 1505) |
| R3589 | 36 | 23 | CUUUCAAGA | AGGAAUACAU | CUUUCAAGACUAA |
| | | | CUAAUAGAU | GGUACACGUU | UAGAUUGCUCCUU |
| | | | UGCUCCUUA | GAA (SEQ ID | ACGAGGAGACAGG |
| | | | CGAGGAGAC | NO: 1489) | AAUACAUGGUACA |
| | | | (SEQ ID NO: | | CGUUGAA (SEQ ID |
| | | | 54) | | NO: 1506) |
| R3590 | 36 | 22 | CUUUCAAGA | AGGAAUACAU | CUUUCAAGACUAA |
| | | | CUAAUAGAU | GGUACACGUU | UAGAUUGCUCCUU |
| | | | UGCUCCUUA | GA (SEQ ID NO: | ACGAGGAGACAGG |
| | | | CGAGGAGAC | 1490) | AAUACAUGGUACA |
| | | | (SEQ ID NO: | | CGUUGA (SEQ ID |
| | | | 54) | | NO: 1507) |
| R3591 | 36 | 21 | CUUUCAAGA | AGGAAUACAU | CUUUCAAGACUAA |
| | | | CUAAUAGAU | GGUACACGUU | UAGAUUGCUCCUU |
| | | | UGCUCCUUA | G (SEQ ID NO: | ACGAGGAGACAGG |
| | | | CGAGGAGAC | 1491) | AAUACAUGGUACA |
| | | | (SEQ ID NO: | | CGUUG (SEQ ID |
| | | | 54) | | NO: 1508) |
| R3592 | 36 | 20 | CUUUCAAGA | AGGAAUACAU | CUUUCAAGACUAA |
| | | | CUAAUAGAU | GGUACACGUU | UAGAUUGCUCCUU |
| | | | UGCUCCUUA | (SEQ ID NO: | ACGAGGAGACAGG |
| | | | CGAGGAGAC | 1492) | AAUACAUGGUACA |
| | | | (SEQ ID NO: | | CGUU (SEQ ID |
| | | | 54) | | NO: 1509) |
| R3593 | 36 | 19 | CUUUCAAGA | AGGAAUACAU | CUUUCAAGACUAA |
| | | | CUAAUAGAU | GGUACACGU | UAGAUUGCUCCUU |
| | | | UGCUCCUUA | (SEQ ID NO: | ACGAGGAGACAGG |
| | | | CGAGGAGAC | 1493) | AAUACAUGGUACA |
| | | | (SEQ ID NO: | | CGU (SEQ ID |
| | | | 54) | | NO: 1510) |
| R3594 | 36 | 18 | CUUUCAAGA | AGGAAUACAU | CUUUCAAGACUAA |
| | | | CUAAUAGAU | GGUACACG | UAGAUUGCUCCUU |
| | | | UGCUCCUUA | (SEQ ID NO: | ACGAGGAGACAGG |
| | | | CGAGGAGAC | 1494) | AAUACAUGGUACA |
| | | | (SEQ ID NO: | | CG (SEQ ID NO: 1511) |
| | | | 54) | | |
| R3595 | 36 | 17 | CUUUCAAGA | AGGAAUACAU | CUUUCAAGACUAA |
| | | | CUAAUAGAU | GGUACAC | UAGAUUGCUCCUU |
| | | | UGCUCCUUA | (SEQ ID NO: | ACGAGGAGACAGG |
| | | | CGAGGAGAC | 1495) | AAUACAUGGUACA |
| | | | (SEQ ID NO: | | C (SEQ ID NO: 1512) |
| | | | 54) | | |
| R3596 | 36 | 16 | CUUUCAAGA | AGGAAUACAU | CUUUCAAGACUAA |
| | | | CUAAUAGAU | GGUACA (SEQ | UAGAUUGCUCCUU |
| | | | UGCUCCUUA | ID NO: 1496) | ACGAGGAGACAGG |
| | | | CGAGGAGAC | | AAUACAUGGUACA |
| | | | (SEQ ID NO: | | (SEQ ID NO: 1513) |
| | | | 54) | | |
| R3597 | 36 | 15 | CUUUCAAGA | AGGAAUACAU | CUUUCAAGACUAA |
| | | | CUAAUAGAU | GGUAC (SEQ ID | UAGAUUGCUCCUU |
| | | | UGCUCCUUA | NO: 1497) | ACGAGGAGACAGG |
| | | | CGAGGAGAC | | AAUACAUGGUAC |
| | | | (SEQ ID NO: | | (SEQ ID NO: 1514) |
| | | | 54) | | |
| R3598 | 35 | 20 | UUUCAAGAC | AGGAAUACAU | UUUCAAGACUAAU |
| | | | UAAUAGAUU | GGUACACGUU | AGAUUGCUCCUUA |
| | | | GCUCCUUAC | (SEQ ID NO: | CGAGGAGACAGGA |
| | | | GAGGAGAC | 1498) | AUACAUGGUACAC |
| | | | (SEQ ID NO: | | GUU (SEQ ID |
| | | | 1466) | | NO: 1515) |
| R3599 | 34 | 20 | UUCAAGACU | AGGAAUACAU | UUCAAGACUAAUA |
| | | | AAUAGAUUG | GGUACACGUU | GAUUGCUCCUUAC |
| | | | CUCCUUACG | (SEQ ID NO: | GAGGAGACAGGAA |
| | | | AGGAGAC | 1498) | UACAUGGUACACG |
| | | | (SEQ ID NO: | | UU (SEQ ID NO: 1516) |
| | | | 1467) | | |
| R3600 | 33 | 20 | UCAAGACUA | AGGAAUACAU | UCAAGACUAAUAG |
| | | | AUAGAUUGC | GGUACACGUU | AUUGCUCCUUACG |
| | | | UCCUUACGA | (SEQ ID NO: | AGGAGACAGGAAU |
| | | | GGAGAC (SEQ | 1498) | ACAUGGUACACGU |
| | | | ID NO: 1468) | | U (SEQ ID NO: 1517) |
| R3601 | 32 | 20 | CAAGACUAA | AGGAAUACAU | CAAGACUAAUAGA |
| | | | UAGAUUGCU | GGUACACGUU | UUGCUCCUUACGA |
| | | | CCUUACGAG | (SEQ ID NO: | GGAGACAGGAAUA |
| | | | GAGAC (SEQ | 1498) | CAUGGUACACGUU |
| | | | ID NO: 1469) | | (SEQ ID NO: 1518) |
| R3602 | 31 | 20 | AAGACUAAU | AGGAAUACAU | AAGACUAAUAGAU |
| | | | AGAUUGCUC | GGUACACGUU | UGCUCCUUACGAG |
| | | | CUUACGAGG | (SEQ ID NO: | GAGACAGGAAUAC |
| | | | AGAC (SEQ ID | 1498) | AUGGUACACGUU |
| | | | NO: 1470) | | (SEQ ID NO: 1519) |
| R3603 | 30 | 20 | AGACUAAUA | AGGAAUACAU | AGACUAAUAGAUU |
| | | | GAUUGCUCC | GGUACACGUU | GCUCCUUACGAGG |
| | | | UUACGAGGA | (SEQ ID NO: | AGACAGGAAUACA |
| | | | GAC (SEQ ID | 1498) | UGGUACACGUU |
| | | | NO: 1471) | | (SEQ ID NO: 1520) |
| R3604 | 29 | 20 | GACUAAUAG | AGGAAUACAU | GACUAAUAGAUUG |
| | | | AUUGCUCCU | GGUACACGUU | CUCCUUACGAGGA |
| | | | UACGAGGAG | (SEQ ID NO: | GACAGGAAUACAU |
| | | | AC (SEQ ID | 1498) | GGUACACGUU (SEQ |
| | | | NO: 1472) | | ID NO: 1521) |
| R3605 | 28 | 20 | ACUAAUAGA | AGGAAUACAU | ACUAAUAGAUUGC |
| | | | UUGCUCCUU | GGUACACGUU | UCCUUACGAGGAG |
| | | | ACGAGGAGA | (SEQ ID NO: | ACAGGAAUACAUG |
| | | | C (SEQ ID NO: | 1498) | GUACACGUU (SEQ |
| | | | 1473) | | ID NO: 1522) |
| R3606 | 27 | 20 | CUAAUAGAU | AGGAAUACAU | CUAAUAGAUUGCU |
| | | | UGCUCCUUA | GGUACACGUU | CCUUACGAGGAGA |
| | | | CGAGGAGAC | (SEQ ID NO: | CAGGAAUACAUGG |
| | | | (SEQ ID NO: | 1498) | UACACGUU (SEQ ID |
| | | | 1474) | | NO: 1523) |
| R3607 | 26 | 20 | UAAUAGAUU | AGGAAUACAU | UAAUAGAUUGCUC |
| | | | GCUCCUUAC | GGUACACGUU | CUUACGAGGAGAC |
| | | | GAGGAGAC | (SEQ ID NO: | AGGAAUACAUGGU |
| | | | (SEQ ID NO: | 1498) | ACACGUU (SEQ ID |
| | | | 1475) | | NO: 1524) |
| R3608 | 25 | 20 | AAUAGAUUG | AGGAAUACAU | AAUAGAUUGCUCC |
| | | | CUCCUUACG | GGUACACGUU | UUACGAGGAGACA |
| | | | AGGAGAC | AGGAAUACAU | GGAAUACAUGGUA |
| | | | (SEQ ID NO: | GGUACACGUU | CACGUU (SEQ ID |
| | | | 1476) | (SEQ ID NO: | NO: 1525) |
| | | | | 2487) | |
| R3609 | 24 | 20 | AUAGAUUGC | AGGAAUACAU | AUAGAUUGCUCCU |
| | | | UCCUUACGA | GGUACACGUU | UACGAGGAGACAG |
| | | | GGAGAC (SEQ | AGGAAUACAU | GAAUACAUGGUAC |
| | | | ID NO: 1477) | GGUACACGUU | ACGUU (SEQ ID |
| | | | | (SEQ ID NO: | NO: 1526) |
| | | | | 2487) | |
| R3610 | 23 | 20 | UAGAUUGCU | AGGAAUACAU | UAGAUUGCUCCUU |
| | | | CCUUACGAG | GGUACACGUU | ACGAGGAGACAGG |
| | | | GAGAC (SEQ | AGGAAUACAU | AAUACAUGGUACA |
| | | | ID NO: 1478) | GGUACACGUU | CGUU (SEQ ID |
| | | | | (SEQ ID NO: | NO: 1527) |
| | | | | 2487) | |
| R3611 | 22 | 20 | AGAUUGCUC | AGGAAUACAU | AGAUUGCUCCUUA |
| | | | CUUACGAGG | GGUACACGUU | CGAGGAGACAGGA |
| | | | AGAC (SEQ ID | AGGAAUACAU | AUACAUGGUACAC |
| | | | NO: 1479) | GGUACACGUU | GUU (SEQ ID |
| | | | | (SEQ ID NO: | NO: 1528) |
| | | | | 2487) | |
| R3612 | 21 | 20 | GAUUGCUCC | AGGAAUACAU | GAUUGCUCCUUAC |
| | | | UUACGAGGA | GGUACACGUU | GAGGAGACAGGAA |
| | | | GAC (SEQ ID | AGGAAUACAU | UACAUGGUACACG |
| | | | NO: 1480) | GGUACACGUU | UU (SEQ ID NO: 1529) |
| | | | | (SEQ ID NO: | |
| | | | | 2487) | |
| R3613 | 20 | 20 | AUUGCUCCU | AGGAAUACAU | AUUGCUCCUUACG |
| | | | UACGAGGAG | GGUACACGUU | AGGAGACAGGAAU |
| | | | AC (SEQ ID | AGGAAUACAU | ACAUGGUACACGU |
| | | | NO: 1481) | GGUACACGUU | U (SEQ ID NO: 1530) |
| | | | | (SEQ ID NO: | |
| | | | | 2487) | |

### Example 34

### Identification of Optimal Guide RNAs for CasΦ Polypeptide-Mediated Genome Editing in Primary Cells

The present example shows identification of the best performing gRNAs that target TRAC, B2M and programmed cell death protein 1 (PD1) in T cells. In this study, CasΦ.12 polypeptides (SEQ ID NO: 107) were incubated with different gRNAs (shown in Table 14) at room temperature for 10 minutes to form RNP complexes. T cells were resuspended at 5×10⁵ cells/20 μL in electroporation solution (Lonza) and an Amaxa 4D Nucleofector with pulse code EH115 was used to nucleofect the cells Immediately after nucleofection, 80 μl pre-warmed culture medium was added to each well. The cells were then left in the cuvette plate for 10 minutes before transfer to the culture plate. After 48 hours, DNA was extracted from half of the cells and PCR was performed to detect the frequency of indels. The rest of the cells were cultured until Day 5, and were then collected for flow cytometry to detect the frequency of TRAC or B2M knockout. FIG. 29A and FIG. 29B show exemplary gRNAs for targeting TRAC. FIG. 29B and FIG. 29C show exemplary gRNAs for targeting B2M. FIG. 29E shows exemplary gRNAs for targeting PD1. Additionally, this example demonstrates that a guide RNAs targeting a non-coding region can mediate gene knockout. For example, R3007, R2995, R2992 and R3014 target non-coding regions of the PD1 gene. The screening for gRNAs targeting TRAC is shown in FIG. 29F and for gRNAs targeting B2M is shown in FIG. 29H. Flow cytometry plots of exemplary gRNAs targeting TRAC are shown in FIG. 29G and of exemplary gRNAs targeting B2M in FIG. 29I.

TABLE 14

| | Tar- | |
|---|---|---|
| Name | get | Spacer sequence (5′ → 3′) |
| R3041 | TRAC | UCCCACAGAUAUCCAGAACC (SEQ ID NO: 2470) |
| R3042 | TRAC | GAGUCUCUCAGCUGGUACAC (SEQ ID NO: 1436) |
| R3043 | TRAC | AGAGUCUCUCAGCUGGUACA (SEQ ID NO: 2471) |
| R3061 | TRAC | AAGUCCAUAGACCUCAUGUC (SEQ ID NO: 2472) |
| R3063 | TRAC | AAGAGCAACAGUGCUGUGGC (SEQ ID NO: 2473) |
| R3066 | TRAC | GUUGCUCCAGGCCACAGCAC (SEQ ID NO: 2474) |
| R3068 | TRAC | GCACAUGCAAAGUCAGAUUU (SEQ ID NO: 2475) |
| R3069 | TRAC | GCAUGUGCAAACGCCUUCAA (SEQ ID NO: 2476) |
| R3081 | TRAC | CUAAAAGGAAAAACAGACAU (SEQ ID NO: 2477) |
| R3141 | TRAC | CUCGACCAGCUUGACAUCAC (SEQ ID NO: 2478) |
| R3088 | B2M | AUAUAAGUGGAGGCGUCGCG (SEQ ID NO: 2479) |
| R3091 | B2M | GGGCCGAGAUGUCUCGCUCC (SEQ ID NO: 1429) |
| R3094 | B2M | UGGCCUGGAGGCUAUCCAGC (SEQ ID NO: 2480) |
| R3119 | B2M | AAGUUGACUUACUGAAGAAU (SEQ ID NO: 2481) |
| R3132 | B2M | AGCAAGGACUGGUCUUUCUA (SEQ ID NO: 2482) |
| R3149 | B2M | AGUGGGGGUGAAUUCAGUGU (SEQ ID NO: 2483) |
| R3150 | B2M | CAGUGGGGGUGAAUUCAGUG (SEQ ID NO: 1434) |
| R3155 | B2M | GGCUGUGACAAAGUCACAUG (SEQ ID NO: 2484) |
| R3156 | B2M | GUCACAGCCCAAGAUAGUUA (SEQ ID NO: 2485) |
| R3157 | B2M | UCACAGCCCAAGAUAGUUAA (SEQ ID NO: 2486) |
| R2946 | PD1 | UGUGACACGGAAGCGGCAGU (SEQ ID NO: 263) |
| R2992 | PD1 | GGGGCUGGUUGGAGAUGGCC (SEQ ID NO: 309) |
| R2995 | PD1 | GAGCAGCCAAGGUGCCCCUG (SEQ ID NO: 312) |
| R3007 | PD1 | ACACAUGCCCAGGCAGCACC (SEQ ID NO: 324) |
| R3014 | PD1 | AGGCCCAGCCAGCACUCUGG (SEQ ID NO: 331) |

### Example 35

### RNP and mRNA Delivery of CasΦ Polypeptides

This example illustrates that CasΦ.12 can be delivered to primary cells as mRNA or as an RNP complex. In one study, RNP complexes were formed using CasΦ.12 protein (0, 100, 200 or 400 pmol) (SEQ ID NO: 107) and gRNAs (0, 400 or 800 pmol) targeting B2M or TRAC. RNP complexes were added to T cells. T cells were nucleofected using the Amaxa P3 kit and Amaxa 4D 96-well electroporation system with pulse code EH115. Cells were harvested for flow cytometry to determine the percentage of B2M or TRAC knockout cells, and genomic DNA was extracted to detect the frequency of indel mutations. As shown in FIG. 30A, a distinct population of B2M-negative cells was detected in T cells transfected with CasΦ.12 RNP complex targeting B2M. A distinct population of TRAC-negative cells was detected in in T cells transfected with CasΦ.12 RNP complex targeting TRAC, and shown in FIG. 30B. Quantification of the percentage of B2M knockout cells is shown in FIG. 30C and quantification of the percentage of TRAC knockout cells is shown in FIG. 30D. A high frequency of indel mutations was also seen after delivery of RNP complexes. As shown in FIG. 30E, ˜55% indel mutations was detected when RNP complexes targeting B2M were formed using 400 pmol protein and 800 pmol guide RNA. A similar frequency of indel mutations was detected when RNP complexes targeting TRAC were formed using the same conditions, as illustrated in FIG. 30F.

In a second study, CasΦ.12 mRNA was delivered to T cells with a gRNA targeting the B2M gene. For nucleofection, T cells were resuspended in BTXpress electroporation medium (5×10⁵ cells per well) and mixed with CasΦ.12 mRNA and 500 pmol gRNA. Cells were collected on Day 2 for extraction of genomic DNA, and the frequency of indel mutations was determined. As shown in FIG. 30G, delivery of CasΦ.12 mRNA and gRNA resulted in a high frequency of indel mutations. This was at a comparable level to genome editing with delivery of Cas9 mRNA. Further data from this study are shown in FIG. 30I and FIG. 30J. FIG. 30I shows the frequency of indel mutations and functional knockout, as assessed by flow cytometry, of the B2M gene induced by either CasΦ.12 or Cas9 targeting the same site. FIG. 30J shows the distribution of the size of indel mutations induced by CasΦ.12 or Cas9 determined by NGS analysis. CasΦ.12 predominantly induced larger deletion mutations whereas Cas9 induced mostly small 1 bp InDels. This data further confirms the ability of CasΦ.12 to mediate genome editing at the B2M locus.

### Example 36

### gRNA Processing by CasΦ Polypeptides in Mammalian Cells

This example illustrates the ability of CasΦ polypeptides to process gRNA in mammalian cells. In this study, HEK293T cells were transfected with crRNA and expression plasmids encoding CasΦ.12 (SEQ ID NO: 107) using lipofectamine on day 0. The crRNA had the repeat sequence (the region that binds to CasΦ.12) CTTTCAAGACTAATAGATTGCTCCTTACGAGGAGAC (SEQ ID NO: 54). To determine the nature of the crRNAs expressed in the HEK293T cells, the microRNA species in the HEK293T cells were analyzed by next generation sequencing. After 2 days, miRNA was extracted using the mirVANA kit. RNA was treated with recombinant Shrimp Alkaline Phosphatase (rSAP) to remove all the phosphates from the 5′ and 3′ ends of the RNA. PNK phosphorylation was then performed to add phosphate back to the 5′ ends in preparation for adaptor ligation to the RNA. RNA was then mixed with 3′ SR Adaptor for Illumina, followed by 3′ ligation enzyme mix and incubated for 1 hour at 25° C. in a thermal cycler. The reverse transcription primer was then hybridized to prevent adaptor-dimer formation. The SR RT primer hybridizes to the excess of 3′ SR Adaptor (that remains free after the 3′ ligation reaction) and transforms the single stranded DNA adaptor into a double-stranded DNA molecule. Double-stranded DNAs are not substrates for ligation mediated by T4 RNA Ligase 1 and therefore do not ligate to the 5′ SR. The RNA-ligation mixture from the previous step was mixed with SR RT primer for Illumina and placed in a thermocycler for the following program: 5 minutes at 75° C., 15 minutes at 37° C., 15 minutes at 25° C., hold at 4° C. The RNA-ligation mixture was then incubated with 5′ SR adaptor for 1 hour at 25° C. in a thermal cycler. Finally, RNA was reverse transcribed using ProtoScript II Reverse Transcriptase and amplified for PCR. The sample was then analyzed by next generation sequencing.

As shown in FIG. 31 the major crRNA molecule detected by sequence analysis was 24 nucleotides long (ATAGATTGCTCCTTACGAGGAGAC (SEQ ID NO: 1531) which is 12 nucleotides shorter than the full length repeat sequence (CTTTCAAGACTAATAGATTGCTCCTTACGAGGAGAC (SED ID NO: 54)) that was delivered to the HEK293T cells. This demonstrates how CasΦ.12 can process the repeat region of its crRNA in mammalian cells.

### Example 37

### CasΦ Polypeptide Cleavage Generates 5′ Overhangs

This example illustrates different CasΦ polypeptide-induced cleavage patterns. In this study, CasΦ polypeptides (CasΦ.12, CasΦ.45, CasΦ.43, CasΦ.39. CasΦ.37, CasΦ.33, CasΦ.32, CasΦ.30, CasΦ.28, CasΦ.25, CasΦ.24, CasΦ.22, CasΦ.20, CasΦ.18) were complexed with a crRNA to form RNPs. The RNPs were then used in cleavage reactions with plasmid DNA comprising a target sequence and a PAM (GTTG). The cleavage reaction was carried out at 37° C. and had a duration of 15 minutes. The cleavage products were then analyzed by gel electrophoresis. As shown in FIG. 32A, the majority of CasΦ polypeptides generated a linear product from a plasmid target, whilst some CasΦ polypeptides introduced nicks into the plasmid DNA.

FIG. 32B shows a schematic of the cut sites on the target and non-target strand of a double-stranded target nucleic acid. The nature of the cleavage patterns resulting from the location of the cut sites on the target and non-target strands was investigated by sequence analysis, as shown in FIG. 32C and represented in FIG. 32D. These data show that the cleavage pattern following CasΦ polypeptide mediated cleavage of target nucleic acid is a staggered cut comprising 5′ overhangs. FIG. 32E shows a table of cut sites and overhangs of the different CasΦ polypeptides. The “#bp overlap” corresponds to the length of the 5′ overhang for each CasΦ polypeptide. For comparison, Cpf1 introduces a staggered double-stranded DNA break with a 4- or 5-nucleotide 5′ overhang (Zetsche et. al 2015 Cell).

### Example 38

### Multiplex Genome Editing with CasΦ Polypeptides

This example illustrates the ability of CasΦ RNP complexes to knockout multiple genes simultaneously. In this study, gRNAs targeting B2M, TRAC and PDCD1 (provided in Table 15) were incubated with CasΦ.12 (SEQ ID NO: 12) for 10 minutes at room temperature to form B2M, TRAC, and PDC1 targeting RNPs, respectively. The B2M targeting RNPs, TRAC targeting RNPs, PDCD1 targeting RNPs and combinations thereof were added to T cells. T cells were resuspended at 5×10⁵ cells/20 μL in Nucleofection P3 solution and an Amaxa 4D 96-well electroporation system with pulse code EH115 was used to nucleofect the cells. Immediately after nucleofection, 85 μl pre-warmed culture medium was added to each well. The cells were then left in the cuvette plate for 10 minutes before transfer to the culture plate. On Day 3, genomic DNA was extracted and sent for NGS sequencing and the % indel was measured with a positive % indel being indicative of % knockout. On Day 5, cells were harvested for flow cytometry and the % knockout was measured with fluorescently labeled antibodies to TRAC and B2M (antibody to PDCD1 unavailable). % indel results are presented in Table 16 and flow cytometry data presented in Table 17. Corresponding flow cytometry panels are shown in FIG. 33.

TABLE 15

| Descrip- | SEQ | |
|---|---|---|
| tion | ID | Sequence |
| B2M | 1532 | CUUUCAAGACUAAUAGAUUGCUCCUUACGAGGAG |
| gRNA | | ACAGCAAGGACUGGUCUUUCUA |
| (R3132) | | |
| TRAC | 1432 | CUUUCAAGACUAAUAGAUUGCUCCUUACGAGGAG |
| gRNA | | ACGAGUCUCUCAGCUGGUACAC |
| (R3042) | | |
| PDCD1 | 791 | CUUUCAAGACUAAUAGAUUGCUCCUUACGAGGAG |
| gRNA | | ACUAGCACCGCCCAGACGACUG |
| (R2925) | | |

TABLE 16

| Description | RNP Guide ID(s) | Amplicon | % INDEL |
|---|---|---|---|
| TRAC single KO | R3042 | TRAC | 77.6% |
| B2M single KO | R3132 | B2M | 85.5% |
| PDCD1 single KO | R2925 | PDCD1 | 44.6% |
| TRAC, B2M double KO | R3132 & R3042 | TRAC | 58.8% |
| TRAC, B2M double KO | R3132 & R3042 | B2M | 61.2% |
| TRAC, B2M, PDCD1 triple | R3132, R3042, | TRAC | 59.2% |
| KO | R2925 | | |
| TRAC, B2M, PDCD1 triple | R3132, R3042, | B2M | 69.4% |
| KO | R2925 | | |
| TRAC, B2M, PDCD1 triple | R3132, R3042, | PDCD1 | 42.1% |
| KO | R2925 | | |

TABLE 17

| | B2M+ | B2M+, | B2M−, | B2M−, |
|---|---|---|---|---|
| gRNA | CD3− | CD3+ | CD3+ | CD3− |
| TRAC | 94 | 5.91 | 0.00418 | 0.1 |
| B2M | 0.051 | 8.65 | 90.7 | 0.59 |
| TRAC + B2M | 4.2 | 4.89 | 4.01 | 86.9 |
| TRAC + B2M + | 4.74 | 14.1 | 4.33 | 76.8 |
| PDCD1 | | | | |

### Example 39

### Genome Editing with CasΦ Polypeptides Mediates Efficient Editing of PCSK9 in Mouse Hepatoma Cells

The present example shows that CasΦ.12 RNP complexes are highly effective at mediating editing the PCSK9 gene. In this study, 95 CasΦ gRNAs targeting PCSK9 (sequences shown in Tables E and Q), were incubated with CasΦ.12 (SEQ ID NO: 12) to form RNP complexes. Positive control RNP complexes were also formed using Cas9 and a gRNA. Hepa1-6 mouse hepatoma cells (100,000 cells) were resuspended in SF solution (Lonza) and nucleofected with CasΦ RNPs (250 pmoles) or the control Cas9 RNPs (60 pmoles) using program CM-137 or CM-148 (Amaxa nucleofector). Cells were collected after 48 hours, genomic DNA was extracted and the frequency of indel mutations was determined using NGS. FIG. 34 shows that CasΦ.12 is a highly effective genome editing tool, with an indel frequency of up to 48% induced by CasΦ.12 RNP complexes. Whereas, the maximum indel frequency induced by Cas9 was only about 22%.

### Example 40

### Adeno-Associated Virus Encoding CasΦ.12 Facilitates Genome Editing

This example shows that a CasΦ.12 plasmid, including both CasΦ polypeptide sequence and gRNA sequence, sometimes called an all-in-one, can be used to facilitate genome editing. In this study, the crRNAs (sequences shown in Tables E and Q) from the initial RNP screen were chosen and truncations of these crRNAs were generated with repeat lengths of 36, 25, 20, or 19 nucleotides in combination with spacer lengths of 20, 17, or 16 nucleotides. Each crRNA was then cloned into an AAV vector consisting of U6 promoter to drive crRNA expression, intron-less EF1alpha short (EFS) promoter driving CasΦ expression, PolyA signal, and 1 kb stuffer sequence genomic. Hepa1-6 mouse hepatoma cells were nucleofected with 10 μg of each AAV plasmid. After 72 hours, genomic DNA was extracted and the frequency of indel mutations was determined using NGS. FIG. 35A shows a plasmid map of the adeno-associated virus (AAV) encoding the CasΦ polypeptide sequence and gRNA sequence. FIG. 35D shows the frequency of CasΦ.12 induced indel mutations in Hepa1-6 cells transduced with 10 μg of each AAV plasmid. gRNAs containing repeat sequences of 19, 20, 25 or 36 nucleotides and spacer sequences of 16, 17 or 20 nucleotides were used in this study. In the graph legend, repeat and spacer lengths are indicated as the number of nucleotides in the repeat followed by the number of nucleotides in the spacer, eg 20-17 has a repeat length of 20 nucleotides and a spacer length of 17 nucleotides. The frequency of indel mutations is comparable to that of Cas9. FIG. 35E and FIG. 35F show the frequency of CasΦ.12 induced indel mutations with different gRNA containing repeat and spacer sequences of different lengths (indicated as in FIG. 35F with repeat length followed by spacer length). This study demonstrates that the all-in-one vector method of CasΦ.12 mediated genome editing is robust across different gRNA sequences and with gRNAs of different repeat and spacer lengths.

AAV vectors are a leading platform for delivery of gene therapy for treatment of human disease (Wang et al., (2019) Nature Reviews Drug Discovery). One of the limitations of viral vector delivery of CRISPR/Cas9 is the size of Cas9. AAVs are roughly 20 nm, allowing for 4.5 kb genomic material to be packaged within it. This makes packaging Cas9 and a gRNA (˜4.2 kB) with any additional elements such as multiple gRNAs or a donor polynucleotide for HDR challenging (Lino et al., (2018), Drug Delivery). Whereas CasΦ is much smaller, allowing all of the components of the CRISPR system to be packaged in one viral vector.

### Example 41

### Optimization of Lipid Nanoparticle Delivery of CasΦ

This example describes the optimization of lipid nanoparticle (LNP) delivery of CasΦ mRNA and gRNA. In this study, the encapsulation efficiency of LNPs was optimized by testing different amine group to phosphate group ratio (N/P) of LNPs containing CasΦ mRNA and gRNA. An LNP kit from Precision Nanosystems (GenVoy-ILM™) was used to generate LNPs with different N/P ratios. LNPs were then dropped into HEK293T cells. Genomic DNA was extracted and the frequency of indel mutations was determined using NGS. The gRNA used in this study was R2470 with 2′ O-methyl on the first three 5′ and last three 3′ nucleotides and phosphorothioate bonds in between the first three 5′ nucleotides and in between the last two 3′ nucleotides. The sequence of R2470 from 5′ to 3′ is 42256-779_601_SL. The mRNA was generated using T7 messenger mRNA IVT kit. As shown in FIG. 36, indel mutations were detected following the use of a range of N/P ratios.

LNPs are one of the most clinically advanced non-viral delivery systems for gene therapy. LNPs have many properties that make them ideal candidates for delivery of nucleic acids, including ease of manufacture, low cytotoxicity and immunogenicity, high efficiency of nucleic acid encapsulation and cell transfection, multidosing capabilities and flexibility of design (Kulkarni et al., (2018) Nucleic Acid Therapeutics).

### Example 42

### Genome Editing in Hematopoietic Stem Cells with CasΦ Polypeptides

This example demonstrates CasΦ-mediated genome editing of CD34+ hematopoietic stem cells (HSCs). HSCs are stem cells that differentiate to give rise blood cells, such as T and B lymphocytes, erythrocytes, monocytes and macrophages. HSCs are important cells for future stem cell therapies as they have the potential to be used to treat genetic blood cell diseases (Morgan et al. (2017), Cell Stem Cell).

In this study human CD34+ cells were grown in XVIVO10 media (+5% FBS, +1X CC 110) for three days. On the third day, the cells were nucleofected using the Lonza P3 kit with either RNP containing CasΦ.12 polypeptides complexed with B2M-targeting guide R3132 (42256-779_601_SL), or a mixture of CasΦ.12 mRNA with B2M-targeting guide. Cells were collected after 3 days, genomic DNA was purified and the frequency of indel mutations at the B2M locus was analyzed by NGS. As shown in FIG. 37, CasΦ.12 is an effective tool for genome editing when CasΦ.12 is delivered to cells as CasΦ.12 RNP complexes or CasΦ.12 mRNA.

This example illustrates the utility of CasΦ polypeptides as genome editing tools in stem cells, such as HSCs.

### Example 43

### Genome Editing in Induced Pluripotent Stem Cells with CasΦ Polypeptides

This example demonstrates CasΦ-mediated genome editing of induced pluripotent stem cells (iPSCs). iPSCs are pluripotent stem cells that are generated from somatic cells. They can propagate indefinitely and give rise to any cell type in the body. These features make iPSCs a powerful tool for researching human disease and provide a promising prospect for cell therapies for a range of medical conditions. iPSCs can be generated in a patient-specific manner and used in autologous transplant, thereby overcoming complications of rejection by the host immune system (Moradi et al. (2019), Stem Cell Research & Therapy).

In this study, high quality WTC-11 iPSCs were harvested as single cells using Accutase treatment for 5 minutes. RNP complexes were formed using CasΦ.12 polypeptides and gRNAs targeting either the B2M locus or targeting a CIITA locus (sequences shown in Table 19). RNP complexes were formed using 2:1 gRNA:CasΦ.12 RNP (1000 pmol gRNA+500 pmol Cas120.12) and incubating at room temperature for approximately 15 minutes. WTC-11 iPSCs (200,000 cells) were resuspended in 20 uL of P3 nucleofection solution per reaction and 40 uL of cell suspension was added to each RNP tube. Half of the volume of each RNP/cell suspension mixture was added to the Lonza 96 well shuttle and nucleofection was performed using the program CD118. To recover the transfected cells, 80 μL of warm StemFlex media supplemented with 2 μM of Thiazovivin was added to the wells of the shuttle. The entire volume of the shuttle well was transferred to a 96 well plate previously coated with 0.337 mg/mL Matrigel containing 100 μL of 2 μM of Thiazovivin. Cells were allowed to recover for 24 hours in 37° C. incubator with humidity control. Cells were confluent 48 hours post-transfection, and single-cell passaged using Accutase. Genomic DNA was extracted using KingFisher Tissue and DNA kit. NGS library preparation was performed using in house protocols and the frequency of indel mutations was quantified using Crispresso. As shown in FIG. 38, effective genome editing at the B2M and CIITA loci was achieved with CasΦ.12 RNP complexes in iPSCs.

This example demonstrates the utility of CasΦ as genome editing tools in iPSCs.

TABLE 19

| | | | SEQ |
|---|---|---|---|
| | Tar- | | ID |
| Name | get | Sequence | NO |
| R3132 | B2M | AUUGCUCCUUACGAGGAGACAGCAAGGACU | 2488 |
| | | GGUCUUU | |
| R4504_ | CIITA | AUUGCUCCUUACGAGGAGACGGGCUCUGAC | 1722 |
| CasPhi12_ | | AGGUAGG | |
| S | | | |
| R5406_ | CIITA | CUUUCAAGACUAAUAGAUUGCUCCUUACGA | 2222 |
| CasPhi12 | | GGAGACGGGUCAAUGCUAGGUACUGC | |

### Example 44

### Genome Editing with CasΦ Polypeptides Mediates Efficient Editing of CIITA Locus

This example demonstrates CasΦ-mediated genome editing of the CIITA locus. In this study, RNP complexes were formed using CasΦ polypeptides and gRNAs targeting CIITA (sequences shown in Tables D and O). K562 cells were nucleofected with RNP complexes (250 pmol) using Lonza nucleofection protocols. Cells were harvested after 48 hours, genomic DNA was isolated and the frequency of indel mutations was evaluated using NGS analysis (MiSeq, Illumina). As shown in FIG. 39, effective genome editing of the CIITA locus was achieved using CasΦ RNP complexes.

While preferred embodiments of the present invention have been shown and described herein, it will be apparent to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

What is claimed is:

1. A composition comprising:
a) a programmable CasΦ nuclease or a nucleic acid encoding said programmable CasΦ nuclease, wherein said programmable CasΦ nuclease comprises at least 85% sequence identity to a sequence selected from the group consisting of SEQ ID NOs: 1 to 47, SEQ ID NO. 105, and SEQ ID NO. 107, and
b) a guide nucleic acid or a nucleic acid encoding said guide nucleic acid, wherein said guide nucleic acid comprises a region comprising a nucleotide sequence that is complementary to a target nucleic acid sequence and an additional region, wherein said region and said additional region are heterologous to each other.

2. The composition of claim 1, wherein the additional region of the guide nucleic acid comprises at least 85% sequence identity to a sequence selected from the group consisting of SEQ ID NOs: 48 to 86.

3. The composition of claim 1, wherein the guide nucleic acid comprises a sequence comprising at least 95% sequence identity to a sequence selected from the group consisting of SEQ ID NOs: 48 to 86.

4. The composition of claim 1, wherein the guide nucleic acid comprises a sequence selected from the group consisting of SEQ ID NOs: 48 to 86.

5. The composition of claim 1, wherein the programmable CasΦ nuclease comprises nickase activity.

6. The composition of claim 1, wherein the programmable CasΦ nuclease comprises double-strand cleavage activity.

7. The composition of claim 1, wherein the programmable CasΦ nuclease comprises at least 90% sequence identity to a sequence selected from the group consisting of SEQ ID NOs: 1 to 47, SEQ ID NO. 105, and SEQ ID NO. 107.

8. The composition of claim 1, wherein the programmable CasΦ nuclease comprises at least 95% sequence identity to a sequence selected from the group consisting of SEQ ID NOs: 1 to 47, SEQ ID NO. 105, and SEQ ID NO. 107.

9. The composition of claim 1, wherein the programmable CasΦ nuclease comprises at least 98% sequence identity to a sequence selected from the group consisting of SEQ ID NOs: 1 to 47, SEQ ID NO. 105, and SEQ ID NO. 107.

10. The composition of claim 1, wherein the programmable CasΦ nuclease comprises a sequence selected from the group consisting of SEQ ID NOs: 1 to 47, SEQ ID NO. 105, and SEQ ID NO. 107.

11. The composition of claim 1, wherein the guide nucleic acid does not comprise a tracrRNA.

12. The composition of claim 1, wherein the programmable CasΦ nuclease comprises greater nickase activity when complexed with the guide nucleic acid at a temperature from about 20° C. to about 25° C., as compared with complex formation at a temperature of about 37° C.

13. The composition of claim 1, wherein the additional region comprises at least 98% sequence identity to SEQ ID NO: 57.

14. The composition of claim 13, wherein the programmable CasΦ nuclease comprises greater nickase activity when complexed with the guide nucleic acid comprising a sequence comprising at least 98% sequence identity to SEQ ID NO: 57, as compared to when complexed with a guide nucleic acid comprising SEQ ID NO: 49.

15. The composition of claim 1, wherein the programmable CasΦ nuclease exhibits greater nicking activity as compared to double stranded cleavage activity.

16. The composition of claim 1, wherein the programmable CasΦ nuclease exhibits greater double stranded cleavage activity as compared to nicking activity.

17. The composition of any one of claims 1-16, wherein the programmable CasΦ nuclease comprises a single active site in a RuvC domain that is capable of catalyzing pre-crRNA processing and nicking or cleaving of nucleic acids.

18. The composition of any one of claims 1-17, wherein the programmable CasΦ nuclease recognizes a protospacer adjacent motif (PAM) of 5′-TBN-3′, wherein B is one or more of C, G, or T.

19. The composition of claim 18, wherein the programmable CasΦ nuclease recognizes a protospacer adjacent motif (PAM) of 5′-TTTN-3′.

20. A method of modifying a target nucleic acid sequence, the method comprising:
contacting a target nucleic acid sequence with a programmable CasΦ nuclease comprising at least 85% sequence identity to a sequence selected from the group consisting of SEQ ID NOs: 1 to 47, SEQ ID NO. 105, and SEQ ID NO. 107, and a guide nucleic acid,
wherein the programmable CasΦ nuclease cleaves the target nucleic acid sequence, thereby modifying the target nucleic acid sequence.

21. The method of claim 20, wherein the programmable CasΦ nuclease introduces a double-stranded break in the target nucleic acid sequence.

22. The method of claim 20, wherein the programmable CasΦ nuclease comprises double-strand cleavage activity.

23. The method of claim 20, wherein the programmable CasΦ nuclease cleaves a single-strand of the target nucleic acid sequence.

24. The method of claim 20, wherein the programmable CasΦ nuclease comprises nickase activity.

25. The method of claim 20, wherein the programmable CasΦ nuclease exhibits greater nicking activity as compared to double stranded cleavage activity.

26. The method of claim 20, wherein the programmable CasΦ nuclease exhibits greater double stranded cleavage activity as compared to nicking activity.

27. The method of claim 20, wherein the target nucleic acid is DNA.

28. The method of claim 20, wherein the target nucleic acid is double-stranded DNA.

29. The method of claim 20, wherein the programmable CasΦ nuclease cleaves a non-target strand of the double-stranded DNA, wherein the non-target strand is non-complementary to the guide nucleic acid.

30. The method of claim 20, wherein the programmable CasΦ nuclease does not cleave a target strand of the double-stranded DNA, wherein the target strand is complementary to the guide nucleic acid.

31. The method of claim 20, wherein the programmable CasΦ nuclease comprises at least 90% sequence identity to a sequence selected from the group consisting of SEQ ID NOs: 1 to 47, SEQ ID NO. 105, and SEQ ID NO. 107.

32. The method of claim 20, wherein the programmable CasΦ nuclease comprises at least 95% sequence identity to a sequence selected from the group consisting of SEQ ID NOs: 1 to 47, SEQ ID NO. 105, and SEQ ID NO. 107.

33. The method of claim 20, wherein the programmable CasΦ nuclease comprises at least 98% sequence identity to a sequence selected from the group consisting of SEQ ID NOs: 1 to 47, SEQ ID NO. 105 and SEQ ID NO. 107.

34. The method of claim 20, wherein the programmable CasΦ nuclease comprises a sequence selected from the group consisting of SEQ ID NOs: 1 to 47, SEQ ID NO. 105, and SEQ ID NO. 107.

35. The method of claim 20, wherein the guide nucleic acid comprises a sequence comprising at least 85% sequence identity to a sequence selected from the group consisting of SEQ ID NOs: 48 to 86.

36. The method of claim 20, wherein the guide nucleic acid comprises a sequence comprising at least 95% sequence identity to a sequence selected from the group consisting of SEQ ID NOs: 48 to 86.

37. The method of claim 20, wherein the guide nucleic acid comprises a sequence selected from the group consisting of SEQ ID NOs: 48 to 86.

38. The method of claim 20, wherein the guide nucleic acid does not comprise a tracrRNA.

39. The method of claim 20, wherein the target nucleic acid sequence comprises a mutated sequence or a sequence associated with a disease.

40. The method of claim 39, wherein the mutated sequence is removed after the programmable CasΦ nuclease cleaves the target nucleic acid sequence.

41. The method of claim 20, wherein the target nucleic acid sequence is in a human cell.

42. The method of claim 20, wherein the method is performed in vivo.

43. The method of claim 20, wherein the method is performed ex vivo.

44. The method of claim 20, further comprising inserting a donor polynucleotide into the target nucleic acid sequence at the site of cleavage.

45. A method of introducing a break in a target nucleic acid, the method comprising: contacting the target nucleic acid with:
(a) a first guide nucleic acid comprising a region that binds to a first programmable nickase comprising at least 85% sequence identity to a sequence selected from the group consisting of SEQ ID NOs: 1 to 47, SEQ ID NO. 105, and SEQ ID NO. 107; and
(b) a second guide nucleic acid comprising a region that binds to a second programmable nickase comprising at least 85% sequence identity to a sequence selected from the group consisting of SEQ ID NOs: 1 to 47, SEQ ID NO. 105, and SEQ ID NO. 107,
wherein the first guide nucleic acid comprises a first additional region that binds to the target nucleic acid and wherein the second guide nucleic acid comprises a second additional region that binds to the target nucleic acid and wherein the first additional region of the first guide nucleic acid and the second additional region of the second guide nucleic acid bind opposing strands of the target nucleic acid.

46. The method of claim 45, wherein the first programmable nickase, the second programmable nickase, or both comprise at least 90% sequence identity to a sequence selected from the group consisting of SEQ ID NOs: 1 to 47, SEQ ID NO. 105, SEQ ID NO. 107.

47. The method of claim 45, wherein the first programmable nickase, the second programmable nickase, or both comprise at least 95% sequence identity to a sequence selected from the group consisting of SEQ ID NOs: 1 to 47, SEQ ID NO. 105, SEQ ID NO. 107.

48. The method of claim 45, wherein the first programmable nickase, the second programmable nickase, or both comprise a sequence selected from the group consisting of SEQ ID NOs: 1 to 47, SEQ ID NO. 105, and SEQ ID NO. 107.

49. The method of claim 45, wherein the first guide nucleic acid, the second guide nucleic acid, or both comprise a sequence comprising at least 85% sequence identity to a sequence selected from the group consisting of SEQ ID NOs: 48 to 86.

50. The method of claim 45, wherein the first guide nucleic acid, the second guide nucleic acid, or both comprise a sequence comprising at least 95% sequence identity to a sequence selected from the group consisting of SEQ ID NOs: 48 to 86.

51. The method of claim 45, wherein the first guide nucleic acid, the second guide nucleic acid, or both comprise a sequence selected from the group consisting of SEQ ID NOs: 48 to 86.

52. The method of claim 45, wherein the first programmable nickase and the second programmable nickase exhibit greater nicking activity as compared to double stranded cleavage activity.

53. The method of claim 45, wherein the first programmable nickase and the second programmable nickase nick the target nucleic acid at two different sites.

54. The method of claim 45, wherein the target nucleic acid comprises double stranded DNA.

55. The method of claim 53, wherein the two different sites are on opposing strands of the double stranded DNA.

56. The method of claim 45, wherein the target nucleic acid comprises a mutated sequence or a sequence is associated with a disease.

57. The method of claim 56, wherein the mutated sequence is removed after the first programmable nickase and the second programmable nickase nick the target nucleic acid.

58. The method of claim 45, wherein the target nucleic acid is in a cell.

59. The method of claim 45, wherein the method is performed in vivo.

60. The method of claim 45, wherein the method is performed ex vivo.

61. The method of any one of claims 45-60, wherein the first programmable nickase and the second programmable nickase are the same.

62. The method of any one of claims 45-60, wherein the first programmable nickase and the second programmable nickase are different.

63. A method of detecting a target nucleic acid in a sample, the method comprising contacting a sample comprising a target nucleic acid with
(a) a programmable CasΦ nuclease comprising at least 85% sequence identity to a sequence selected from the group consisting of SEQ ID NOs: 1 to 47, SEQ ID NO. 105, and SEQ ID NO. 105;
(b) a guide RNA comprising a region that binds to the programmable CasΦ nuclease and an additional region that binds to the target nucleic acid; and
(c) a labeled single stranded DNA reporter that does not bind the guide RNA;
cleaving the labeled single stranded DNA reporter by the programmable CasΦ nuclease to release a detectable label; and
detecting the target nucleic acid by measuring a signal from the detectable label.

64. The method of claim 63, wherein the target nucleic acid is single stranded DNA.

65. The method of claim 63, wherein the target nucleic acid is double stranded DNA.

66. The method of claim 63, wherein the target nucleic acid is a viral nucleic acid.

67. The method of claim 63, wherein the target nucleic acid is bacterial nucleic acid.

68. The method of claim 63, wherein the target nucleic acid is from a human cell.

69. The method of claim 63, wherein the target nucleic acid is a fetal nucleic acid.

70. The method of claim 63, wherein the sample is derived from a subject's saliva, blood, serum, plasma, urine, aspirate, or biopsy sample.

71. The method of claim 63, wherein the programmable CasΦ nuclease comprises at least 95% sequence identity to a sequence selected from the group consisting of SEQ ID NOs: 1 to 47, SEQ ID NO. 105, SEQ ID NO. 107.

72. The method of claim 63, wherein the programmable CasΦ nuclease comprises a sequence selected from the group consisting of SEQ ID NOs: 1 to 47, SEQ ID NO. 105, and SEQ ID NO. 107.

73. The method of claim 63, wherein the guide RNA comprises at least about 95% sequence identity to a sequence selected from the group consisting of SEQ ID NOs: 48 to 86.

74. The method of claim 63, wherein the guide RNA comprises a sequence selected from the group consisting of SEQ ID NOs: 48 to 86.

75. The method of claim 63, wherein the sample comprises a phosphate buffer, a Tris buffer, or a HEPES buffer.

76. The method of claim 63, wherein the sample comprises a pH of 7 to 9.

77. The method of claim 63, wherein the sample comprises a pH of 7.5 to 8.

78. The method of claim 63, wherein the sample comprises a salt concentration of 25 nM to 200 mM.

79. The method of claim 63, wherein the single stranded DNA reporter comprises an ssDNA-fluorescence quenching DNA reporter.

80. The method of claim 63, wherein the ssDNA-fluorescence quenching DNA reporter is a universal ssDNA-fluorescence quenching DNA reporter.

81. The method of claim 63, wherein the programmable CasΦ nuclease exhibits PAM-independent cleaving.

82. A method of modulating transcription of a gene in a cell, the method comprising: introducing into a cell comprising a target nucleic acid sequence:
(i) a fusion polypeptide or a nucleic acid encoding the fusion polypeptide, wherein the fusion polypeptide comprises:
(a) a dCasΦ polypeptide comprising at least 85% sequence identity to a sequence selected from the group consisting of SEQ ID NOs: 1 to 47, SEQ ID NO. 105, and SEQ ID NO. 107, wherein the dCasΦ polypeptide is enzymatically inactive; and
(b) a polypeptide comprising transcriptional regulation activity; and
(ii) a guide nucleic acid, or a nucleic acid comprising a nucleotide sequence encoding the guide nucleic acid, wherein the guide nucleic acid comprises a region that binds to the dCasΦ polypeptide and an additional region that binds to the target nucleic acid;
wherein transcription of the gene is modulated through the fusion polypeptide acting on the target nucleic acid sequence.

83. The method of claim 82, wherein the dCasΦ polypeptide comprises at least 95% sequence identity to a sequence selected from the group consisting of SEQ ID NOs: 1 to 47, SEQ ID NO. 105, and SEQ ID NO. 107.

84. The method of claim 82, wherein the guide nucleic acid comprises at least about 95% sequence identity to a sequence selected from the group consisting of SEQ ID NOs: 48 to 86.

85. The method of claim 82, wherein the guide nucleic acid comprises a sequence selected from the group consisting of SEQ ID NOs: 48 to 86.

86. The method of claim 82, wherein the guide nucleic acid comprises a sequence selected from the group consisting of SEQ ID NOs: 48 to 86.

87. The method of claim 82, wherein the polypeptide comprising transcriptional regulation activity polypeptide comprises transcription activation activity.

88. The method of claim 82, wherein the polypeptide comprising transcriptional regulation activity polypeptide comprises transcription repressor activity.

89. The method of claim 82, wherein the polypeptide comprising transcriptional regulation activity polypeptide comprises an activity selected from the group consisting of transcription activation activity, transcription repression activity, nuclease activity, transcription release factor activity, histone modification activity, histone acetyltransferase activity, nucleic acid association activity, DNA methylase activity, direct or indirect DNA demethylase activity, methyltransferase activity, demethylase activity, acetyltransferase activity, deacetylase activity, kinase activity, phosphatase activity, ubiquitin ligase activity, deubiquitinating activity, adenylation activity, deadenylation activity, deaminase activity, SUMOylating activity, deSUMOylating activity, ribosylation activity, deribosylation activity, myristoylation activity, and demyristoylation activity.

90. A composition comprising:
a) a Cas nuclease or nucleic acid encoding said Cas nuclease, and
b) a guide nucleic acid or a nucleic acid encoding said guide nucleic acid, wherein said guide nucleic acid comprises a region comprising a nucleotide sequence that is complementary to a target nucleic acid sequence and an additional region, wherein said region and said additional region are heterologous to each other;
wherein the Cas nuclease comprises a RuvC domain, wherein the RuvC domain is capable of processing a pre-crRNA and cleaving the target nucleic acid.

91. The composition of claim 90, wherein the same active site in the RuvC domain catalyzes the processing of the pre-crRNA and the cleaving of the target nucleic acid.

92. The composition of claims 90 or 91, wherein the Cas nuclease is the programmable CasΦ nuclease of any one of claims 1-18.

93. The composition of any one of claims 90-92, wherein the Cas nuclease recognizes a protospacer adjacent motif (PAM) of 5′-TBN-3′, wherein B is one or more of C, G, or, T.

94. The composition of claim 93, wherein the Cas nuclease recognizes a protospacer adjacent motif (PAM) of 5′-TTTN-3′, optionally wherein the PAM is 5′-TTTN-3′.

95. The composition of claim 93, wherein the PAM is 5′-GTTK-3′, 5′-VTTK-3′, 5′-VTTS-3′, 5′-TTTS-3′ or 5′-VTTN-3′, where K is G or T, V is A, C or G, and S is C or G.

96. The composition of any one of claims 90-94, wherein the composition is used in a method of any one of claims 20-89.

97. The use of a programmable CasΦ nuclease to modify a target nucleic acid sequence according to the method of claims 20 to 44.

98. The use of a first programmable nickase and a second programmable nickase to introduce a break in a target nucleic acid according to the method of claims 45 to 62.

99. The use of a programmable CasΦ nuclease to detect a target nucleic acid in a sample according to the method of claims 63 to 81.

100. The use of a dCasΦ polypeptide to modulate transcription of a gene in a cell according to the method of claims 82 to 89.

101. The composition of any one of claims 1-19 or 45-100, wherein the region is a spacer region and the additional region is a repeat region.

102. The method, composition, or use of any one of claims 1-19 or 45-100, wherein the region is a repeat region and the additional region is a spacer region.

103. The method, composition, or use of claim 101 or 102, wherein the repeat region comprises a GAC sequence, optionally wherein the GAC sequence is at the 3′ end of the repeat region.

104. The method, composition, or use of claims 101-103, wherein the repeat region comprises a hairpin, optionally wherein the hairpin is in the 3′ portion of the repeat region.

105. The method, composition, or use of claim 104, wherein the hairpin comprises a double-stranded stem portion and a single-stranded loop portion.

106. The method, composition, or use of claim 105, wherein a strand of the stem portion comprises a CYC sequence and the other strand of the stem portion comprises a GRG sequence, wherein Y and R are complementary.

107. The method, composition, or use of claim 106, wherein the G of the GAC sequence is in the stem portion of the hairpin.

108. The method, composition, or use of any one of claims 105-107, wherein each strand of the stem portion comprises 3, 4 or 5 nucleotides.

109. The method, composition, or use of any one of claims 105-108, wherein the loop portion comprises between 2 and 8 nucleotides, optionally wherein the loop portion comprises 4 nucleotides.

110. The composition of claim 1, wherein the guide nucleic acid comprises at least 98% sequence identity to SEQ ID NO: 54.

111. The method, composition, or use according to any one of claims 101-110, wherein the repeat region is between 15 and 50 nucleotides in length, preferably, wherein the repeat region is between 19 and 37 nucleotides in length.

112. The method, composition, or use according to any one of claims 101-111, wherein the spacer region is between 15 and 50 nucleotides in length, between 15 and 40 nucleotides in length, or between 15 and 35 nucleotides in length, preferably wherein the spacer region is between 16 and 30 nucleotides in length.

113. The method, composition, or use according to claim 112, wherein the spacer region is between 16 and 20 nucleotides in length.

114. The composition according to any one of claims 1-19, 90-95, 101-113, wherein the programmable CasΦ nuclease forms a complex with a divalent metal ion, preferably wherein the divalent metal ion is Mg²⁺.

115. A programmable CasΦ nuclease or a nucleic acid encoding said programmable CasΦ nuclease, wherein said programmable CasΦ nuclease comprises at least 85% sequence identity to a sequence selected from the group consisting of SEQ ID NOs: 1 to 47, SEQ ID NO. 105, and SEQ ID NO. 107, and wherein
a) the programmable CasΦ nuclease is capable of binding to a guide RNA comprising a first region that is complementary to a target nucleic acid sequence in a eukaryotic genome and a second region that binds to the programmable CasΦ nuclease;
b) a complex comprising the programmable CasΦ nuclease and the guide RNA binds to the target sequence;
c) the programmable CasΦ nuclease comprises a RuvC domain, wherein the RuvC domain is capable of processing a pre-crRNA and cleaving the target nucleic acid; and
d) the programmable CasΦ nuclease does not require a tracrRNA to cleave the target nucleic acid.

116. A programmable CasΦ nuclease or a nucleic acid encoding said programmable CasΦ nuclease, wherein said programmable CasΦ nuclease comprises a RuvC-like domain which matches PFAM family PF07282 and does not match PFAM family PF18516, and wherein
a) the programmable CasΦ nuclease is capable of binding to a guide RNA comprising a first region that is complementary to a target nucleic acid sequence in a eukaryotic genome and a second region that binds to the programmable CasΦ nuclease;
b) a complex comprising the programmable CasΦ nuclease and the guide RNA binds to the target sequence;
c) the RuvC-like domain is capable of processing a pre-crRNA and cleaving the target nucleic acid; and
d) the programmable CasΦ nuclease does not require a tracrRNA to cleave the target nucleic acid.

117. A programmable CasΦ nuclease or a nucleic acid encoding said programmable CasΦ nuclease, wherein said programmable CasΦ nuclease comprises at least 85% sequence identity to a sequence selected from the group consisting of SEQ ID NOs: 1 to 47, SEQ ID NO. 105, or SEQ ID NO. 107, and wherein
a) the programmable CasΦ nuclease comprises a RuvC-like domain which matches PFAM family PF07282 and does not match PFAM family PF18516;
b) the programmable CasΦ nuclease is capable of binding to a guide RNA comprising a first region that is complementary to a target nucleic acid sequence in a eukaryotic genome and a second region that binds to the programmable CasΦ nuclease;
c) a complex comprising the programmable CasΦ nuclease and the guide RNA binds to the target sequence;
d) the RuvC-like domain is capable of processing a pre-crRNA and cleaving the target nucleic acid; and
e) the programmable CasΦ nuclease does not require a tracrRNA to cleave the target nucleic acid.

118. The programmable CasΦ nuclease or a nucleic acid of claims 115-117, wherein the same active site in the RuvC domain or RuvC-like domain catalyzes the processing of the pre-crRNA and the cleaving of the target nucleic acid.

119. The programmable CasΦ nuclease or a nucleic acid of claims 115-118, wherein the programmable CasΦ nuclease is fused or linked to one or more NLS.

120. The programmable CasΦ nuclease or a nucleic acid of claims 115-119, wherein:
a) the one or more NLS are fused or linked to the N-terminus of the programmable CasΦ nuclease;
b) the one or more NLS are fused or linked to the C-terminus of the programmable CasΦ nuclease; or
c) the one or more NLS are fused or linked to the N-terminus and the C-terminus of the programmable CasΦ nuclease.

121. A composition comprising the programmable CasΦ nuclease or a nucleic acid of claims 115-120 and a gRNA comprising a first region that is complementary to a target nucleic acid sequence in a eukaryotic genome and a second region that binds to the programmable CasΦ nuclease.

122. A composition comprising the programmable CasΦ nuclease or a nucleic acid of claims 115-120 and a cell, preferably wherein the cell is a eukaryotic cell.

123. A composition comprising the programmable CasΦ nuclease or a nucleic acid of claims 115-120 and a gRNA comprising a first region that is complementary to a target nucleic acid sequence in a eukaryotic genome and a second region that binds to the programmable CasΦ nuclease and a cell, preferably wherein the cell is a eukaryotic cell.

124. A eukaryotic cell comprising the programmable CasΦ nuclease or a nucleic acid of claims 115-120.

125. The eukaryotic cell of claim 124, wherein the cell further comprises a gRNA comprising a first region that is complementary to a target nucleic acid sequence in a eukaryotic genome and a second region that binds to the programmable CasΦ nuclease and a cell, preferably wherein the cell is a eukaryotic cell.

126. A vector comprising the nucleic acid of claims 115-120.

127. The vector of claim 126, wherein the vector is a viral vector.

128. The composition of claim 18, wherein the programmable CasΦ nuclease recognizes a protospacer adjacent motif (PAM) of 5′-TTN-3′.

129. The composition of any one of claims 1-17, wherein the programmable CasΦ nuclease recognizes a protospacer adjacent motif (PAM) of 5′-GTTB-3′, wherein B is C, G, or T.

130. The composition of claim 93, wherein the Cas nuclease recognizes a protospacer adjacent motif (PAM) of 5′-TTN-3′, optionally wherein the PAM is 5′-TTN-3′.

131. The composition of any one of claims 90-94, wherein the Cas nuclease recognizes a protospacer adjacent motif (PAM) of 5′-GTTK-3′, 5′-VTTK-3′, 5′-VTTS-3′, 5′-TTTS-3′ or 5′-VTTN-3′, where K is G or T, V is A, C or G, and S is C or G.

132. The composition of any one of claims 90-94, wherein the Cas nuclease recognizes a protospacer adjacent motif (PAM) of 5′-GTTB-3′, wherein B is C, G, or T.

133. A programmable CasΦ nuclease or a nucleic acid encoding said programmable CasΦ nuclease, wherein said programmable CasΦ nuclease comprises at least 85% sequence identity to a sequence selected from the group consisting of SEQ ID NOs: 1 to 47, SEQ ID NO. 105, and SEQ ID NO. 107, and wherein
a) the programmable CasΦ nuclease is capable of binding to a guide RNA comprising a first region that is complementary to a target nucleic acid sequence in a eukaryotic genome and a second region that binds to the programmable CasΦ nuclease;
b) a complex comprising the programmable CasΦ nuclease and the guide RNA binds to the target sequence;
c) the programmable CasΦ nuclease comprises a RuvC domain, wherein the RuvC domain is capable of processing a pre-crRNA and cleaving the target nucleic acid;
d) the programmable CasΦ nuclease cleaves both strands of the target nucleic acid comprising the target sequence, wherein the strand break is a staggered cut with a 5′ overhang; and
e) the programmable CasΦ nuclease does not require a tracrRNA to cleave the target nucleic acid.

134. A programmable CasΦ nuclease or a nucleic acid encoding said programmable CasΦ nuclease, wherein said programmable CasΦ nuclease comprises a RuvC-like domain which matches PFAM family PF07282 and does not match PFAM family PF18516, and wherein
a) the programmable CasΦ nuclease is capable of binding to a guide RNA comprising a first region that is complementary to a target nucleic acid sequence in a eukaryotic genome and a second region that binds to the programmable CasΦ nuclease;
b) a complex comprising the programmable CasΦ nuclease and the guide RNA binds to the target sequence;
c) the RuvC-like domain is capable of processing a pre-crRNA and cleaving the target nucleic acid;
d) the programmable CasΦ nuclease cleaves both strands of the target nucleic acid comprising the target sequence, wherein the strand break is a staggered cut with a 5′ overhang; and
e) the programmable CasΦ nuclease does not require a tracrRNA to cleave the target nucleic acid.

135. A programmable nuclease or a nucleic acid encoding said programmable nuclease, wherein said programmable nuclease is a Type V CRISPR/Cas enzyme nuclease and comprises between 400 and 900 amino acids, and wherein
a) the programmable CasΦ nuclease is capable of binding to a guide RNA comprising a first region that is complementary to a target nucleic acid sequence in a eukaryotic genome and a second region that binds to the programmable CasΦ nuclease;
b) a complex comprising the programmable CasΦ nuclease and the guide RNA binds to the target sequence;
c) the programmable CasΦ nuclease comprises a RuvC domain, wherein the RuvC domain is capable of processing a pre-crRNA and cleaving the target nucleic acid;
d) the programmable CasΦ nuclease cleaves both strands of the target nucleic acid comprising the target sequence, wherein the strand break is a staggered cut with a 5′ overhang; and
e) the programmable CasΦ nuclease does not require a tracrRNA to cleave the target nucleic acid.

136. A programmable CasΦ nuclease or a nucleic acid encoding said programmable CasΦ nuclease, wherein said programmable CasΦ nuclease comprises at least 85% sequence identity to a sequence selected from the group consisting of SEQ ID NOs: 1 to 47, SEQ ID NO. 105, and SEQ ID NO. 107, and wherein
a) the programmable CasΦ nuclease is capable of binding to a guide RNA comprising a first region that is complementary to a target nucleic acid sequence in a eukaryotic genome and a second region that binds to the programmable CasΦ nuclease;
b) a complex comprising the programmable CasΦ nuclease and the guide RNA binds to the target sequence;
c) the programmable CasΦ nuclease comprises a RuvC domain, wherein the RuvC domain is capable of processing a pre-crRNA and cleaving the target nucleic acid;
d) the programmable CasΦ nuclease is capable of cleaving the second region of the guide RNA in mammalian cells; and
e) the programmable CasΦ nuclease does not require a tracrRNA to cleave the target nucleic acid.

137. A programmable CasΦ nuclease or a nucleic acid encoding said programmable CasΦ nuclease, wherein said programmable CasΦ nuclease comprises a RuvC-like domain which matches PFAM family PF07282 and does not match PFAM family PF18516, and wherein
a) the programmable CasΦ nuclease is capable of binding to a guide RNA comprising a first region that is complementary to a target nucleic acid sequence in a eukaryotic genome and a second region that binds to the programmable CasΦ nuclease;
b) a complex comprising the programmable CasΦ nuclease and the guide RNA binds to the target sequence;
c) the RuvC-like domain is capable of processing a pre-crRNA and cleaving the target nucleic acid;
d) the programmable CasΦ nuclease is capable of cleaving the second region of the guide RNA in mammalian cells; and
e) the programmable CasΦ nuclease does not require a tracrRNA to cleave the target nucleic acid.

138. A programmable nuclease or a nucleic acid encoding said programmable nuclease, wherein said programmable nuclease is a Type V CRISPR/Cas enzyme nuclease and comprises between 400 and 900 amino acids, and wherein
a) the programmable CasΦ nuclease is capable of binding to a guide RNA comprising a first region that is complementary to a target nucleic acid sequence in a eukaryotic genome and a second region that binds to the programmable CasΦ nuclease;
b) a complex comprising the programmable CasΦ nuclease and the guide RNA binds to the target sequence;
c) the RuvC-like domain is capable of processing a pre-crRNA and cleaving the target nucleic acid;
d) the programmable CasΦ nuclease is capable of cleaving the second region of the guide RNA in mammalian cells; and
e) the programmable CasΦ nuclease does not require a tracrRNA to cleave the target nucleic acid.

139. A programmable CasΦ nuclease or a nucleic acid encoding said programmable CasΦ nuclease, wherein said programmable CasΦ nuclease comprises at least 85% sequence identity to a sequence selected from the group consisting of SEQ ID NOs: 1 to 47, SEQ ID NO. 105, and SEQ ID NO. 107, and wherein
a) the programmable CasΦ nuclease is capable of binding to a guide RNA comprising a first region that is complementary to a target nucleic acid sequence in a eukaryotic genome and a second region that binds to the programmable CasΦ nuclease;
b) a complex comprising the programmable CasΦ nuclease and the guide RNA binds to the target sequence;
c) the programmable CasΦ nuclease comprises a RuvC domain, wherein the RuvC domain is capable of processing a pre-crRNA and cleaving the target nucleic acid;
d) the programmable CasΦ nuclease cleaves both strands of a target nucleic acid comprising the target sequence, wherein the strand break is a staggered cut with a 5′ overhang;
e) the programmable CasΦ nuclease is capable of cleaving the second region of the guide RNA in mammalian cells; and
f) the programmable CasΦ nuclease does not require a tracrRNA to cleave the target nucleic acid.

140. A programmable CasΦ nuclease or a nucleic acid encoding said programmable CasΦ nuclease, wherein said programmable CasΦ nuclease comprises a RuvC-like domain which matches PFAM family PF07282 and does not match PFAM family PF18516, and wherein
a) the programmable CasΦ nuclease is capable of binding to a guide RNA comprising a first region that is complementary to a target nucleic acid sequence in a eukaryotic genome and a second region that binds to the programmable CasΦ nuclease;
b) a complex comprising the programmable CasΦ nuclease and the guide RNA binds to the target sequence;
c) the RuvC-like domain is capable of processing a pre-crRNA and cleaving the target nucleic acid;
d) the programmable CasΦ nuclease cleaves both strands of a target nucleic acid comprising the target sequence, wherein the strand break is a staggered cut with a 5′ overhang;
e) the programmable CasΦ nuclease is capable of cleaving the second region of the guide RNA in mammalian cells; and
f) the programmable CasΦ nuclease does not require a tracrRNA to cleave the target nucleic acid.

141. A programmable nuclease or a nucleic acid encoding said programmable nuclease, wherein said programmable nuclease is a Type V CRISPR/Cas enzyme nuclease and comprises between 400 and 900 amino acids, and wherein
a) the programmable CasΦ nuclease is capable of binding to a guide RNA comprising a first region that is complementary to a target nucleic acid sequence in a eukaryotic genome and a second region that binds to the programmable CasΦ nuclease;
b) a complex comprising the programmable CasΦ nuclease and the guide RNA binds to the target sequence;
c) the RuvC-like domain is capable of processing a pre-crRNA and cleaving the target nucleic acid;
d) the programmable CasΦ nuclease cleaves both strands of a target nucleic acid comprising the target sequence, wherein the strand break is a staggered cut with a 5′ overhang;
e) the programmable CasΦ nuclease is capable of cleaving the second region of the guide RNA in mammalian cells; and
f) the programmable CasΦ nuclease does not require a tracrRNA to cleave the target nucleic acid.

142. A programmable CasΦ nuclease or a nucleic acid encoding said programmable CasΦ nuclease, wherein said programmable CasΦ nuclease comprises at least 85% sequence identity to a sequence selected from the group consisting of SEQ ID NOs: 1 to 47, SEQ ID NO. 105, and SEQ ID NO. 107, and wherein
a) the programmable CasΦ nuclease is capable of binding to a guide RNA comprising a first region that is complementary to a target nucleic acid sequence in a eukaryotic genome and a second region that binds to the programmable CasΦ nuclease, wherein the first region comprises a seed region comprising between 10 and 16 nucleosides;
b) a complex comprising the programmable CasΦ nuclease and the guide RNA binds to the target sequence;
c) the programmable CasΦ nuclease comprises a RuvC domain, wherein the RuvC domain is capable of processing a pre-crRNA and cleaving the target nucleic acid; and
d) the programmable CasΦ nuclease does not require a tracrRNA to cleave the target nucleic acid.

143. A programmable CasΦ nuclease or a nucleic acid encoding said programmable CasΦ nuclease, wherein said programmable CasΦ nuclease comprises a RuvC-like domain which matches PFAM family PF07282 and does not match PFAM family PF18516, and wherein
a) the programmable CasΦ nuclease is capable of binding to a guide RNA comprising a first region that is complementary to a target nucleic acid sequence in a eukaryotic genome and a second region that binds to the programmable CasΦ nuclease, wherein the first region comprises a seed region comprising between 10 and 16 nucleosides;
b) a complex comprising the programmable CasΦ nuclease and the guide RNA binds to the target sequence;
c) the RuvC-like domain is capable of processing a pre-crRNA and cleaving the target nucleic acid; and
d) the programmable CasΦ nuclease does not require a tracrRNA to cleave the target nucleic acid.

144. A programmable nuclease or a nucleic acid encoding said programmable nuclease, wherein said programmable nuclease is a Type V CRISPR/Cas enzyme nuclease and comprises between 400 and 900 amino acids, and wherein
a) the programmable CasΦ nuclease is capable of binding to a guide RNA comprising a first region that is complementary to a target nucleic acid sequence in a eukaryotic genome and a second region that binds to the programmable CasΦ nuclease, wherein the first region comprises a seed region comprising between 10 and 16 nucleosides;
b) a complex comprising the programmable CasΦ nuclease and the guide RNA binds to the target sequence;
c) the RuvC-like domain is capable of processing a pre-crRNA and cleaving the target nucleic acid; and
d) the programmable CasΦ nuclease does not require a tracrRNA to cleave the target nucleic acid.

145. A programmable CasΦ nuclease or a nucleic acid encoding said programmable CasΦ nuclease, wherein said programmable CasΦ nuclease comprises at least 85% sequence identity to a sequence selected from the group consisting of SEQ ID NOs: 1 to 47, SEQ ID NO. 105, and SEQ ID NO. 107, and wherein
a) the programmable CasΦ nuclease is capable of binding to a guide RNA comprising a first region that is complementary to a target nucleic acid sequence in a eukaryotic genome and a second region that binds to the programmable CasΦ nuclease, wherein the first region comprises a seed region comprising between 10 and 16 nucleosides;
b) a complex comprising the programmable CasΦ nuclease and the guide RNA binds to the target sequence;
c) the programmable CasΦ nuclease comprises a RuvC domain, wherein the RuvC domain is capable of processing a pre-crRNA and cleaving the target nucleic acid;
d) the programmable CasΦ nuclease cleaves both strands of the target nucleic acid comprising the target sequence, wherein the strand break is a staggered cut with a 5′ overhang; and
e) the programmable CasΦ nuclease does not require a tracrRNA to cleave the target nucleic acid.

146. A programmable CasΦ nuclease or a nucleic acid encoding said programmable CasΦ nuclease, wherein said programmable CasΦ nuclease comprises a RuvC-like domain which matches PFAM family PF07282 and does not match PFAM family PF18516, and wherein
a) the programmable CasΦ nuclease is capable of binding to a guide RNA comprising a first region that is complementary to a target nucleic acid sequence in a eukaryotic genome and a second region that binds to the programmable CasΦ nuclease, wherein the first region comprises a seed region comprising between 10 and 16 nucleosides;
b) a complex comprising the programmable CasΦ nuclease and the guide RNA binds to the target sequence;
c) the RuvC-like domain is capable of processing a pre-crRNA and cleaving the target nucleic acid;
d) the programmable CasΦ nuclease cleaves both strands of the target nucleic acid comprising the target sequence, wherein the strand break is a staggered cut with a 5′ overhang; and
e) the programmable CasΦ nuclease does not require a tracrRNA to cleave the target nucleic acid.

147. A programmable nuclease or a nucleic acid encoding said programmable nuclease, wherein said programmable nuclease is a Type V CRISPR/Cas enzyme nuclease and comprises between 400 and 900 amino acids, and wherein
a) the programmable CasΦ nuclease is capable of binding to a guide RNA comprising a first region that is complementary to a target nucleic acid sequence in a eukaryotic genome and a second region that binds to the programmable CasΦ nuclease, wherein the first region comprises a seed region comprising between 10 and 16 nucleosides;
b) a complex comprising the programmable CasΦ nuclease and the guide RNA binds to the target sequence;
c) the RuvC-like domain is capable of processing a pre-crRNA and cleaving the target nucleic acid;
d) the programmable CasΦ nuclease cleaves both strands of the target nucleic acid comprising the target sequence, wherein the strand break is a staggered cut with a 5′ overhang; and
e) the programmable CasΦ nuclease does not require a tracrRNA to cleave the target nucleic acid.

148. A programmable CasΦ nuclease or a nucleic acid encoding said programmable CasΦ nuclease, wherein said programmable CasΦ nuclease comprises at least 85% sequence identity to a sequence selected from the group consisting of SEQ ID NOs: 1 to 47, SEQ ID NO. 105, and SEQ ID NO. 107, and wherein
a) the programmable CasΦ nuclease is capable of binding to a guide RNA comprising a first region that is complementary to a target nucleic acid sequence in a eukaryotic genome and a second region that binds to the programmable CasΦ nuclease, wherein the first region comprises a seed region comprising between 10 and 16 nucleosides;
b) a complex comprising the programmable CasΦ nuclease and the guide RNA binds to the target sequence;
c) the programmable CasΦ nuclease comprises a RuvC domain, wherein the RuvC domain is capable of processing a pre-crRNA and cleaving the target nucleic acid;
d) the programmable CasΦ nuclease is capable of cleaving the second region of the guide RNA in mammalian cells; and
e) the programmable CasΦ nuclease does not require a tracrRNA to cleave the target nucleic acid.

149. A programmable CasΦ nuclease or a nucleic acid encoding said programmable CasΦ nuclease, wherein said programmable CasΦ nuclease comprises a RuvC-like domain which matches PFAM family PF07282 and does not match PFAM family PF18516, and wherein
a) the programmable CasΦ nuclease is capable of binding to a guide RNA comprising a first region that is complementary to a target nucleic acid sequence in a eukaryotic genome and a second region that binds to the programmable CasΦ nuclease, wherein the first region comprises a seed region comprising between 10 and 16 nucleosides;
b) a complex comprising the programmable CasΦ nuclease and the guide RNA binds to the target sequence;
c) the RuvC-like domain is capable of processing a pre-crRNA and cleaving the target nucleic acid;
d) the programmable CasΦ nuclease is capable of cleaving the second region of the guide RNA in mammalian cells; and
e) the programmable CasΦ nuclease does not require a tracrRNA to cleave the target nucleic acid.

150. A programmable nuclease or a nucleic acid encoding said programmable nuclease, wherein said programmable nuclease is a Type V CRISPR/Cas enzyme nuclease and comprises between 400 and 900 amino acids, and wherein
a) the programmable CasΦ nuclease is capable of binding to a guide RNA comprising a first region that is complementary to a target nucleic acid sequence in a eukaryotic genome and a second region that binds to the programmable CasΦ nuclease, wherein the first region comprises a seed region comprising between 10 and 16 nucleosides;
b) a complex comprising the programmable CasΦ nuclease and the guide RNA binds to the target sequence;
c) the RuvC-like domain is capable of processing a pre-crRNA and cleaving the target nucleic acid;
d) the programmable CasΦ nuclease is capable of cleaving the second region of the guide RNA in mammalian cells; and
e) the programmable CasΦ nuclease does not require a tracrRNA to cleave the target nucleic acid.

151. A programmable CasΦ nuclease or a nucleic acid encoding said programmable CasΦ nuclease, wherein said programmable CasΦ nuclease comprises at least 85% sequence identity to a sequence selected from the group consisting of SEQ ID NOs: 1 to 47, SEQ ID NO. 105, and SEQ ID NO. 107, and wherein
a) the programmable CasΦ nuclease is capable of binding to a guide RNA comprising a first region that is complementary to a target nucleic acid sequence in a eukaryotic genome and a second region that binds to the programmable CasΦ nuclease, wherein the first region comprises a seed region comprising between 10 and 16 nucleosides;
b) a complex comprising the programmable CasΦ nuclease and the guide RNA binds to the target sequence;
c) the programmable CasΦ nuclease comprises a RuvC domain, wherein the RuvC domain is capable of processing a pre-crRNA and cleaving the target nucleic acid;
d) the programmable CasΦ nuclease cleaves both strands of a target nucleic acid comprising the target sequence, wherein the strand break is a staggered cut with a 5′ overhang;
e) the programmable CasΦ nuclease is capable of cleaving the second region of the guide RNA in mammalian cells; and
f) the programmable CasΦ nuclease does not require a tracrRNA to cleave the target nucleic acid.

152. A programmable CasΦ nuclease or a nucleic acid encoding said programmable CasΦ nuclease, wherein said programmable CasΦ nuclease comprises a RuvC-like domain which matches PFAM family PF07282 and does not match PFAM family PF18516, and wherein
a) the programmable CasΦ nuclease is capable of binding to a guide RNA comprising a first region that is complementary to a target nucleic acid sequence in a eukaryotic genome and a second region that binds to the programmable CasΦ nuclease, wherein the first region comprises a seed region comprising between 10 and 16 nucleosides;
b) a complex comprising the programmable CasΦ nuclease and the guide RNA binds to the target sequence;
c) the RuvC-like domain is capable of processing a pre-crRNA and cleaving the target nucleic acid;
d) the programmable CasΦ nuclease cleaves both strands of a target nucleic acid comprising the target sequence, wherein the strand break is a staggered cut with a 5′ overhang;
e) the programmable CasΦ nuclease is capable of cleaving the second region of the guide RNA in mammalian cells; and
f) the programmable CasΦ nuclease does not require a tracrRNA to cleave the target nucleic acid.

153. A programmable nuclease or a nucleic acid encoding said programmable nuclease, wherein said programmable nuclease is a Type V CRISPR/Cas enzyme nuclease and comprises between 400 and 900 amino acids, and wherein
a) the programmable CasΦ nuclease is capable of binding to a guide RNA comprising a first region that is complementary to a target nucleic acid sequence in a eukaryotic genome and a second region that binds to the programmable CasΦ nuclease, wherein the first region comprises a seed region comprising between 10 and 16 nucleosides;
b) a complex comprising the programmable CasΦ nuclease and the guide RNA binds to the target sequence;
c) the RuvC-like domain is capable of processing a pre-crRNA and cleaving the target nucleic acid;
d) the programmable CasΦ nuclease cleaves both strands of a target nucleic acid comprising the target sequence, wherein the strand break is a staggered cut with a 5′ overhang;
e) the programmable CasΦ nuclease is capable of cleaving the second region of the guide RNA in mammalian cells; and
f) the programmable CasΦ nuclease does not require a tracrRNA to cleave the target nucleic acid.

154. The programmable CasΦ nuclease or a nucleic acid of any of claims 133-153, wherein the same active site in the RuvC domain or RuvC-like domain catalyzes the processing of the pre-crRNA and the cleaving of the target nucleic acid.

155. The programmable CasΦ nuclease or a nucleic acid of any of claims 133-154, wherein the programmable CasΦ nuclease is fused or linked to one or more NLS.

156. The programmable CasΦ nuclease or a nucleic acid of any of claims 133-155, wherein:
a) the one or more NLS are fused or linked to the N-terminus of the programmable CasΦ nuclease;
b) the one or more NLS are fused or linked to the C-terminus of the programmable CasΦ nuclease; or
c) the one or more NLS are fused or linked to the N-terminus and the C-terminus of the programmable CasΦ nuclease.

157. A composition comprising the programmable CasΦ nuclease or a nucleic acid of any of claims 133-156 and a gRNA comprising a first region that is complementary to a target nucleic acid sequence in a eukaryotic genome and a second region that binds to the programmable CasΦ nuclease.

158. The composition of claim 157, wherein the first region comprises a seed region comprising between 10 and 16 nucleosides.

159. The composition of claim 158, wherein the seed region comprises 16 nucleosides.

160. A composition comprising the programmable CasΦ nuclease or a nucleic acid of claims 133-156 and a cell, preferably wherein the cell is a eukaryotic cell.

161. A composition comprising the programmable CasΦ nuclease or a nucleic acid of any of claims 133-156 and a gRNA comprising a first region that is complementary to a target nucleic acid sequence in a eukaryotic genome and a second region that binds to the programmable CasΦ nuclease and a cell, preferably wherein the cell is a eukaryotic cell.

162. The composition of claim 161, wherein the first region comprises a seed region comprising between 10 and 16 nucleosides.

163. The composition of claim 162, wherein the seed region comprises 16 nucleosides.

164. A eukaryotic cell comprising the programmable CasΦ nuclease or a nucleic acid of any of claims 133-156.

165. The eukaryotic cell of claim 164, wherein the cell further comprises a gRNA comprising a first region that is complementary to a target nucleic acid sequence in a eukaryotic genome and a second region that binds to the programmable CasΦ nuclease.

166. The eukaryotic cell of claim 165, wherein the first region comprises a seed region comprising between 10 and 16 nucleosides.

167. The eukaryotic cell of claim 166, wherein the seed region comprises 16 nucleosides.

168. A vector comprising the nucleic acid of any of claims 133-156.

169. The vector of claim 168, wherein the vector is a viral vector.

170. A guide nucleic acid, or a nucleic acid encoding said guide nucleic acid, comprising a sequence that is the same as or differs by no more than 5, 4, 3, 2, or 1 nucleotides from:
a) a sequence from Tables A to AH; or
b) a sequence comprising a repeat sequence from Table 2 and a spacer sequence from Tables A to H.

171. The guide nucleic acid of claim 170 comprising:
a) a sequence from Tables A to AH; or
b) a sequence comprising a repeat sequence from Table 2 and a spacer sequence from Tables A to H.

172. The guide nucleic acid of claim 170 or claim 171, wherein the guide nucleic acid comprises RNA and/or DNA.

173. The guide nucleic acid of claim 172, wherein the guide nucleic acid is a guide RNA.

174. A complex comprising the guide nucleic acid of any of claims 171 to 173 and a programmable CasΦ nuclease.

175. A eukaryotic cell comprising the guide nucleic acid of any of claims 165 to 167.

176. The eukaryotic cell of claim 175 further comprising a programmable CasΦ nuclease.

177. A vector encoding the guide nucleic acid of any of claims 170 to 173.

178. The vector of claim 177, wherein the vector is a viral vector.

179. A method of introducing a first modification in a first gene and a second modification in a second gene, the method comprising contacting a cell with a CasΦ nuclease; a first guide RNA that is at least partially complementary to an equal length portion of the first gene; and a second guide RNA that is at least partially complementary to an equal length portion of the second gene.

180. The method of claim 179, wherein the CasΦ nuclease is a CasΦ12 nuclease.

181. The method of claim 180, wherein the CasΦ12 nuclease comprises or consists of an amino acid sequence of SEQ ID NO: 12.

182. The method of any one of claims 179-181, wherein the first and/or second modification comprises an insertion of a nucleotide, a deletion of a nucleotide or a combination thereof.

183. The method of any one of claims 179-181, wherein the first and/or second modification comprises an epigenetic modification.

184. The method of any one of claims 179-183, wherein the first and/or second mutation results in a reduction in the expression of the first gene and/or second gene, respectively.

185. The method of any one of claims 179-184, wherein the reduction in the expression is at least about a 10% reduction, at least about a 20% reduction, at least about a 30% reduction, at least about a 40% reduction, at least about a 50% reduction, at least about a 60% reduction, at least about a 70% reduction, at least about an 80% reduction, or at least about a 90% reduction.

186. The method of any one of claims 179-185, comprising contacting the cell with three different guide RNAs targeting three different genes.

187. A programmable CasΦ nuclease or a nucleic acid encoding said programmable CasΦ nuclease, wherein said programmable CasΦ nuclease comprises at least 85% sequence identity to SEQ ID NO: 12.

188. The programmable CasΦ nuclease or a nucleic acid of claim 187, wherein said programmable CasΦ nuclease comprises at least 90% sequence identity to SEQ ID NO: 12.

189. The programmable CasΦ nuclease or a nucleic acid of claim 187, wherein said programmable CasΦ nuclease comprises at least 95% sequence identity to SEQ ID NO: 12.

190. The programmable CasΦ nuclease or a nucleic acid of claim 187, wherein said programmable CasΦ nuclease comprises at least 98% sequence identity to SEQ ID NO: 12.

191. The programmable CasΦ nuclease or a nucleic acid of claim 187, wherein said programmable CasΦ nuclease comprises or consists of an amino acid sequence of SEQ ID NO: 12.

192. A programmable CasΦ nuclease or a nucleic acid encoding said programmable CasΦ nuclease, wherein said programmable CasΦ nuclease comprises at least 85% sequence identity to SEQ ID NO: 18.

193. The programmable CasΦ nuclease or a nucleic acid of claim 192, wherein said programmable CasΦ nuclease comprises at least 90% sequence identity to SEQ ID NO: 18.

194. The programmable CasΦ nuclease or a nucleic acid of claim 192, wherein said programmable CasΦ nuclease comprises at least 95% sequence identity to SEQ ID NO: 18.

195. The programmable CasΦ nuclease or a nucleic acid of claim 192, wherein said programmable CasΦ nuclease comprises at least 98% sequence identity to SEQ ID NO: 18.

196. The programmable CasΦ nuclease or a nucleic acid of claim 192, wherein said programmable CasΦ nuclease comprises or consists of an amino acid sequence of SEQ ID NO: 18.

197. A programmable CasΦ nuclease or a nucleic acid encoding said programmable CasΦ nuclease, wherein said programmable CasΦ nuclease comprises at least 85% sequence identity to SEQ ID NO: 32.

198. The programmable CasΦ nuclease or a nucleic acid of claim 197, wherein said programmable CasΦ nuclease comprises at least 85% sequence identity to SEQ ID NO: 32.

199. The programmable CasΦ nuclease or a nucleic acid of claim 197, wherein said programmable CasΦ nuclease comprises at least 90% sequence identity to SEQ ID NO: 32.

200. The programmable CasΦ nuclease or a nucleic acid of claim 197, wherein said programmable CasΦ nuclease comprises at least 95% sequence identity to SEQ ID NO: 32.

201. The programmable CasΦ nuclease or a nucleic acid of claim 197, wherein said programmable CasΦ nuclease comprises at least 98% sequence identity to SEQ ID NO: 32.

202. The programmable CasΦ nuclease or a nucleic acid of claim 197, wherein said programmable CasΦ nuclease comprises or consists of an amino acid sequence of SEQ ID NO: 32.

203. The programmable CasΦ nuclease or a nucleic acid of any one of claims 187 to 202, wherein the programmable CasΦ nuclease is capable of binding to a guide RNA comprising a first region that is complementary to a target nucleic acid sequence in a eukaryotic genome and a second region that binds to the programmable CasΦ nuclease.

204. The programmable CasΦ nuclease or a nucleic acid of claim 203, wherein a complex comprising the programmable CasΦ nuclease and the guide RNA binds to the target sequence.

205. The programmable CasΦ nuclease or a nucleic acid of any one of claims 187 to 204, wherein the programmable CasΦ nuclease does not require a tracrRNA to cleave a target nucleic acid.

206. The programmable CasΦ nuclease or a nucleic acid of any one of claims 187 to 205, wherein the programmable CasΦ nuclease wherein the programmable CasΦ nuclease comprises a RuvC domain, wherein the RuvC domain is capable of processing a pre-crRNA and cleaving a target nucleic acid.

207. A composition comprising the programmable CasΦ nuclease or a nucleic acid of any of claims 187-206 and a guide nucleic acid comprising a first region that is complementary to a target nucleic acid sequence in a eukaryotic genome and a second region that binds to the programmable CasΦ nuclease.

208. The composition of claim 207, wherein the first region comprises a seed region comprising between 10 and 16 nucleosides.

209. The composition of claim 209, wherein the seed region comprises 16 nucleosides.

210. A composition comprising the programmable CasΦ nuclease or a nucleic acid of claims 187-206 and a cell, preferably wherein the cell is a eukaryotic cell.

211. A composition comprising the programmable CasΦ nuclease or a nucleic acid of any of claims 187-206 and a guide nucleic acid comprising a first region that is complementary to a target nucleic acid sequence in a eukaryotic genome and a second region that binds to the programmable CasΦ nuclease and a cell, preferably wherein the cell is a eukaryotic cell.

212. The composition of claim 211, wherein the first region comprises a seed region comprising between 10 and 16 nucleosides.

213. The composition of claim 212, wherein the seed region comprises 16 nucleosides.

214. A eukaryotic cell comprising the programmable CasΦ nuclease or a nucleic acid of any of claims 187-206.

215. The eukaryotic cell of claim 214, wherein the cell further comprises a guide nucleic acid comprising a first region that is complementary to a target nucleic acid sequence in a eukaryotic genome and a second region that binds to the programmable CasΦ nuclease.

216. The eukaryotic cell of claim 215, wherein the first region comprises a seed region comprising between 10 and 16 nucleosides.

217. The eukaryotic cell of claim 217, wherein the seed region comprises 16 nucleosides.

218. A vector comprising the nucleic acid of any of claims 187-206.

219. The vector of claim 218, wherein the vector is a viral vector.

220. The vector of claim 168 or claim 218, wherein the vector further comprises a nucleic acid encoding a guide nucleic acid, wherein the guide nucleic acid comprises a first region that is complementary to a target nucleic acid sequence in a eukaryotic genome and a second region that binds to the programmable CasΦ nuclease.

221. The vector of claim 220, wherein the guide nucleic acid is a guide RNA.

222. The vector of any one of claims 168, 219-221, wherein the further comprises a donor polynucleotide.

223. The composition of claim 207 or claim 211 or the eukaryotic cell of claim 215, wherein the guide nucleic acid is a guide RNA.

224. A programmable nuclease or a nucleic acid encoding said programmable nuclease, wherein said programmable nuclease is a Type V CRISPR/Cas enzyme nuclease and comprises between 400 and 900 amino acids, and wherein
a) the programmable nuclease is capable of binding to a guide RNA comprising a first region that is complementary to a target nucleic acid sequence in a eukaryotic genome and a second region that binds to the programmable nuclease;
b) a complex comprising the programmable nuclease and the guide RNA binds to the target sequence;
c) the programmable nuclease comprises a RuvC domain, wherein the RuvC domain is capable of processing a pre-crRNA and cleaving the target nucleic acid;
d) the programmable nuclease cleaves both strands of the target nucleic acid comprising the target sequence, wherein the strand break is a staggered cut with a 5′ overhang; and
e) the programmable nuclease does not require a tracrRNA to cleave the target nucleic acid.

225. A programmable nuclease or a nucleic acid encoding said programmable nuclease, wherein said programmable nuclease is a Type V CRISPR/Cas enzyme nuclease and comprises between 400 and 900 amino acids, and wherein
a) the programmable nuclease is capable of binding to a guide RNA comprising a first region that is complementary to a target nucleic acid sequence in a eukaryotic genome and a second region that binds to the programmable nuclease;
b) a complex comprising the programmable nuclease and the guide RNA binds to the target sequence;
c) the RuvC-like domain is capable of processing a pre-crRNA and cleaving the target nucleic acid;
d) the programmable nuclease is capable of cleaving the second region of the guide RNA in mammalian cells; and
e) the programmable nuclease does not require a tracrRNA to cleave the target nucleic acid.

226. A programmable nuclease or a nucleic acid encoding said programmable nuclease, wherein said programmable nuclease is a Type V CRISPR/Cas enzyme nuclease and comprises between 400 and 900 amino acids, and wherein
a) the programmable nuclease is capable of binding to a guide RNA comprising a first region that is complementary to a target nucleic acid sequence in a eukaryotic genome and a second region that binds to the programmable nuclease;
b) a complex comprising the programmable nuclease and the guide RNA binds to the target sequence;
c) the RuvC-like domain is capable of processing a pre-crRNA and cleaving the target nucleic acid;
d) the programmable nuclease cleaves both strands of a target nucleic acid comprising the target sequence, wherein the strand break is a staggered cut with a 5′ overhang;
e) the programmable nuclease is capable of cleaving the second region of the guide RNA in mammalian cells; and
f) the programmable nuclease does not require a tracrRNA to cleave the target nucleic acid.

227. A programmable nuclease or a nucleic acid encoding said programmable nuclease, wherein said programmable nuclease is a Type V CRISPR/Cas enzyme nuclease and comprises between 400 and 900 amino acids, and wherein
a) the programmable nuclease is capable of binding to a guide RNA comprising a first region that is complementary to a target nucleic acid sequence in a eukaryotic genome and a second region that binds to the programmable nuclease, wherein the first region comprises a seed region comprising between 10 and 16 nucleosides;
b) a complex comprising the programmable nuclease and the guide RNA binds to the target sequence;
c) the RuvC-like domain is capable of processing a pre-crRNA and cleaving the target nucleic acid; and
d) the programmable nuclease does not require a tracrRNA to cleave the target nucleic acid.

228. A programmable nuclease or a nucleic acid encoding said programmable nuclease, wherein said programmable nuclease is a Type V CRISPR/Cas enzyme nuclease and comprises between 400 and 900 amino acids, and wherein
a) the programmable nuclease is capable of binding to a guide RNA comprising a first region that is complementary to a target nucleic acid sequence in a eukaryotic genome and a second region that binds to the programmable nuclease, wherein the first region comprises a seed region comprising between 10 and 16 nucleosides;
b) a complex comprising the programmable nuclease and the guide RNA binds to the target sequence;
c) the RuvC-like domain is capable of processing a pre-crRNA and cleaving the target nucleic acid;
d) the programmable nuclease cleaves both strands of the target nucleic acid comprising the target sequence, wherein the strand break is a staggered cut with a 5′ overhang; and
e) the programmable nuclease does not require a tracrRNA to cleave the target nucleic acid.

229. A programmable nuclease or a nucleic acid encoding said programmable nuclease, wherein said programmable nuclease is a Type V CRISPR/Cas enzyme nuclease and comprises between 400 and 900 amino acids, and wherein
a) the programmable nuclease is capable of binding to a guide RNA comprising a first region that is complementary to a target nucleic acid sequence in a eukaryotic genome and a second region that binds to the programmable nuclease, wherein the first region comprises a seed region comprising between 10 and 16 nucleosides;
b) a complex comprising the programmable nuclease and the guide RNA binds to the target sequence;
c) the RuvC-like domain is capable of processing a pre-crRNA and cleaving the target nucleic acid;
d) the programmable nuclease is capable of cleaving the second region of the guide RNA in mammalian cells; and
e) the programmable nuclease does not require a tracrRNA to cleave the target nucleic acid.

230. A programmable nuclease or a nucleic acid encoding said programmable nuclease, wherein said programmable nuclease is a Type V CRISPR/Cas enzyme nuclease and comprises between 400 and 900 amino acids, and wherein
a) the programmable nuclease is capable of binding to a guide RNA comprising a first region that is complementary to a target nucleic acid sequence in a eukaryotic genome and a second region that binds to the programmable nuclease, wherein the first region comprises a seed region comprising between 10 and 16 nucleosides;
b) a complex comprising the programmable nuclease and the guide RNA binds to the target sequence;
c) the RuvC-like domain is capable of processing a pre-crRNA and cleaving the target nucleic acid;
d) the programmable nuclease cleaves both strands of a target nucleic acid comprising the target sequence, wherein the strand break is a staggered cut with a 5′ overhang;
e) the programmable nuclease is capable of cleaving the second region of the guide RNA in mammalian cells; and
f) the programmable nuclease does not require a tracrRNA to cleave the target nucleic acid.

231. The programmable nuclease or a nucleic acid of any of claims 224-230, wherein the same active site in the RuvC domain or RuvC-like domain catalyzes the processing of the pre-crRNA and the cleaving of the target nucleic acid.

232. The programmable nuclease or a nucleic acid of any of claims 224-231, wherein the programmable nuclease is fused or linked to one or more NLS.

233. The programmable nuclease or a nucleic acid of claim 232, wherein:
a) the one or more NLS are fused or linked to the N-terminus of the programmable nuclease;
b) the one or more NLS are fused or linked to the C-terminus of the programmable nuclease; or
c) the one or more NLS are fused or linked to the N-terminus and the C-terminus of the programmable nuclease.

234. A composition comprising the programmable nuclease or a nucleic acid of any of claims 224-233 and a gRNA comprising a first region that is complementary to a target nucleic acid sequence in a eukaryotic genome and a second region that binds to the programmable nuclease.

235. The composition of claim 234, wherein the first region comprises a seed region comprising between 10 and 16 nucleosides.

236. The composition of claim 235, wherein the seed region comprises 16 nucleosides.

237. A composition comprising the programmable nuclease or a nucleic acid of claims 224-233 and a cell, preferably wherein the cell is a eukaryotic cell.

238. A composition comprising the programmable nuclease or a nucleic acid of any of claims 224-233 and a gRNA comprising a first region that is complementary to a target nucleic acid sequence in a eukaryotic genome and a second region that binds to the programmable nuclease and a cell, preferably wherein the cell is a eukaryotic cell.

239. The composition of claim 238, wherein the first region comprises a seed region comprising between 10 and 16 nucleosides.

240. The composition of claim 239, wherein the seed region comprises 16 nucleosides.

241. A eukaryotic cell comprising the programmable nuclease or a nucleic acid of any of claims 224-233.

242. The eukaryotic cell of claim 241, wherein the cell further comprises a gRNA comprising a first region that is complementary to a target nucleic acid sequence in a eukaryotic genome and a second region that binds to the programmable nuclease.

243. The eukaryotic cell of claim 242, wherein the first region comprises a seed region comprising between 10 and 16 nucleosides.

244. The eukaryotic cell of claim 243, wherein the seed region comprises 16 nucleosides.

245. A vector comprising the nucleic acid of any of claims 224-233.

246. The vector of claim 245, wherein the vector is a viral vector.

247. A complex comprising a first programmable CasΦ nuclease and a second programmable CasΦ nuclease.

248. The complex of claim 224, wherein the first programmable CasΦ nuclease and the second programmable CasΦ nuclease are the same programmable CasΦ nuclease.

249. A dimer comprising a first programmable CasΦ nuclease and a second programmable CasΦ nuclease.

250. A homodimer comprising a first programmable CasΦ nuclease and a second programmable CasΦ nuclease.

251. A method of modifying a cell comprising a target nucleic acid, comprising introducing the composition of any one of claims 1-19, 90-95, 157-159, 207-209, 234-236 to the cell, wherein the programmable CasΦ nuclease, programmable nuclease or the cas nuclease cleaves the target nucleic acid, thereby modifying the cell.

252. A method of modifying a cell comprising a target nucleic acid, comprising introducing to the cell (i) the programmable CasΦ nuclease or programmable nuclease of any one of claims 115-120, 133-156, 187-206, or 224-233 and (ii) a guide nucleic acid, wherein the programmable CasΦ nuclease or programmable Cas nuclease cleaves the target nucleic acid, thereby modifying the cell.

253. The method of claim 252, wherein the guide nucleic acid is a guide RNA.

254. The method of any one of claims 251-253, wherein the method further comprises introducing a donor polynucleotide to the cell.

255. The method of claim 254, wherein the method comprises inserting the donor polynucleotide into the target nucleic acid at the site of cleavage.

256. The method of any one of claims 251-255, wherein the cell is a eukaryotic cell, preferably a human cell.

257. The method of claim 256, wherein the cell is a T cell.

258. The method of claim 257, wherein the T cell is a CAR-T cell.

259. The method of claim 256, wherein the cell is a stem cell.

260. The method of claim 259, wherein the cell is a hematopoietic stem cell.

261. The method of claim 259, wherein the stem cell is a pluripotent stem cell, preferably an induced pluripotent stem cell.

262. A modified cell obtained or obtainable by the method of any one of claims 251-261.

263. A modified human cell obtained or obtainable by the method of claim 41.

264. A modified cell obtained or obtainable by the method of claim 58.

265. The modified cell of claim 264, wherein the cell is a eukaryotic cell, preferably a human cell.

266. The modified cell of any one of claims 263-265, wherein the cell is a T cell.

267. The modified cell of claim 266, wherein the T cell is a CAR-T cell.

268. The modified cell of any one of claims 263-265, wherein the cell is a stem cell.

269. The modified cell of claim 268, wherein the cell is a hematopoietic stem cell.

270. The modified cell of claim 268, wherein the cell is a pluripotent stem cell, preferably an induced pluripotent stem cell.

271. The use of a CasΦ nuclease to introduce a first modification in a first gene and a second modification in a gene according to the method of any one of claims 179 to 186.

272. The use of a programmable CasΦ nuclease, programmable nuclease or a cas nuclease to modify a cell according to the method of any one of claims 251 to 261.

273. The method of claim 251 or claim 252, wherein the introducing comprises lipid nanoparticle delivery of nucleic acid encoding the programmable CasΦ nuclease, programmable nuclease or cas nuclease and the guide nucleic acid.

274. The method of claim 273, wherein the nucleic acid further comprises a donor polynucleotide.

275. The method of claim 273 or claim 274, wherein the nucleic acid is a viral vector.

276. The method of claim 275, wherein the viral vector is an AAV vector.
